(19) United States
(12) Patent Application Publication
(10) Pub. No.: US 2024/0287453 A1
(43) Pub. Date: Aug. 29, 2024

(54) PERSISTENT ALLOGENEIC MODIFIED IMMUNE CELLS AND METHODS OF USE THEREOF

(71) Applicant: Beam Therapeutics Inc., Cambridge, MA (US)

(72) Inventors: Colby MALDINI, Cambridge, MA (US); Angelica MESSANA, Cambridge, MA (US); Jason Michael GEHRKE, Cambridge, MA (US); Leila PERARO, Cambridge, MA (US); Lauren YOUNG, Cambridge, MA (US)

(73) Assignee: Beam Therapeutics Inc., Cambridge, MA (US)

(21) Appl. No.: 18/441,740

(22) Filed: Feb. 14, 2024

Related U.S. Application Data
(63) Continuation of application No. PCT/US2022/075021, filed on Aug. 16, 2022.
(60) Provisional application No. 63/233,648, filed on Aug. 16, 2021.
(60) Provisional application No. 63/293,692, filed on Dec. 24, 2021.
(60) Provisional application No. 63/293,722, filed on Dec. 24, 2021.
(60) Provisional application No. 63/336,109, filed on Apr. 28, 2022.

(51) Int. Cl. C12N 5/078; A61K 35/17; C12N 15/11; C12N 15/85

(52) U.S. Cl. CPC C12N 5/0634; A61K 35/17; C12N 15/111; C12N 15/85; C12N 2510/00

(57) ABSTRACT

The present disclosure features allogeneic modified immune cells (e.g., T- or NK-cells) having increased persistence, increased resistance to immune rejection, or decreased risk of eliciting a host-versus-graft reaction, or a combination thereof. Methods for producing and using the same are also provided.

### CROSS-REFERENCE TO RELATED APPLICATIONS

This application is a continuation under 35 U.S.C. § 111(a) of PCT International Patent Application No. PCT/US2022/075021, filed Aug. 16, 2022, designating the United States and published in English, which claims priority to and the benefit of U.S. Provisional Application No. 63/233,648, filed Aug. 16, 2021, U.S. Provisional Application No. 63/293,692, filed Dec. 24, 2021, U.S. Provisional Application No. 63/293,722, filed Dec. 24, 2021, and U.S. Provisional Application No. 63/336,109, filed Apr. 28, 2022, the entire contents of all of which are incorporated by reference herein.

### SEQUENCE LISTING

The contents of the electronic sequence listing (180802-043604US_SL.xml; Size: 3,199,295 bytes; and Date of Creation: May 15, 2024) is herein incorporated by reference in its entirety.

### BACKGROUND OF THE INVENTION

Autologous and allogeneic immunotherapies are neoplasia treatment approaches in which immune cells expressing chimeric antigen receptors are administered to a subject. To generate an immune cell that expresses a chimeric antigen receptor (CAR), the immune cell is first collected from the subject (autologous) or a donor separate from the subject receiving treatment (allogeneic) and genetically modified to express the chimeric antigen receptor. The resulting cell expresses the chimeric antigen receptor on its cell surface (e.g., CAR-T cell), and upon administration to the subject, the chimeric antigen receptor binds to the marker expressed by the neoplastic cell. This interaction with the marker activates the CAR-T cell, which then kills the neoplastic cell. But for autologous or allogeneic cell therapy to be effective and efficient, significant conditions and cellular responses, such as T cell signaling inhibition, must be overcome or avoided. Autologous cell therapies have numerous disadvantages associated with having to usually obtain the starting material from the patient to be treated, including long manufacturing times and the requirement that the patient cells are suitable despite previous therapies or disease state. However, for allogeneic cell therapy, graft-versus-host disease (GVHD) and host rejection of CAR-T cells provide additional challenges. Currently, allogeneic T cells show limited persistence in patients. Thus, there is a significant need for techniques to increase the persistence of allogeneic CAR-T cells and other modified immune cells in vivo.

### SUMMARY OF THE INVENTION

By leveraging, e.g., base editing, the present disclosure shows that allogeneic immune cells can be modified to be resistant to T-cell-based or NK cell-based immune rejection. The present disclosure also features engineered allogeneic modified immune cells (e.g., T- or NK-cells) having increased persistence, increased resistance to immune rejection, and/or decreased risk of eliciting a host-versus-graft reaction, and methods of producing and using such cells, for example, in the treatment of neoplasias without the disadvantages of autologous cell therapies such as long manufacturing times, or the need for an adequate supply of sufficiently healthy autologous donor cells.

In one aspect, the invention of the disclosure features a method for producing a persistent allogeneic modified immune cell. The method involves contacting a cell with a base editor containing a polynucleotide programmable DNA binding polypeptide (napDNAbp), a deaminase, and one or more guide RNAs (gRNAs) that target the base editor to effect an alteration in a nucleic acid molecule, thereby producing a persistent allogeneic modified immune cell. The nucleic acid molecule encodes a polypeptide and/or contains a regulatory element associated with expression thereof. The polypeptide is selected from one or more of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP).

In another aspect, the invention of the disclosure features a method for producing a persistent allogeneic modified immune cell. The method involves contacting a cell with a base editor containing a polynucleotide programmable DNA binding polypeptide (napDNAbp), a deaminase, and guide RNAs (gRNAs) that target the base editor to effect an alteration in one or more nucleic acid molecules, where the one or more nucleic acid molecules encode the following polypeptides and/or contain regulatory elements associated with expression thereof. CD5, B2M, CD3 gamma, CD3 epsilon, CIITA, and PD-1 (PD1), thereby producing the persistent allogeneic modified immune cell.

In another aspect, the invention of the disclosure features a method for producing a persistent allogeneic modified immune cell. The method involves contacting a cell with a base editor containing a polynucleotide programmable DNA binding polypeptide (napDNAbp), a deaminase, and guide RNAs (gRNAs) that target the base editor to effect an alteration in one or more nucleic acid molecules, thereby producing the persistent allogeneic modified immune cell. The one or more nucleic acid molecules encode the following polypeptides and/or comprise regulatory elements associated with expression thereof: HLA-A, HLA-B, and CIITA. The persistent allogeneic modified immune cell surface-expresses HLA-C.

In another aspect, the invention of the disclosure features a method for producing a persistent allogeneic modified immune cell. The method involves contacting a cell with a base editor containing a polynucleotide programmable DNA binding polypeptide (napDNAbp), a deaminase, and two or more guide RNAs (gRNAs) that target the base editor to effect an alteration in two or more nucleic acid molecules, thereby producing a persistent allogeneic modified immune cell. The nucleic acid molecules encode a polypeptide and/or contain a regulatory element associated with expression thereof. A first polypeptide is selected from the one or more of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP). The second polypeptide is selected from one or more of beta-2 microglobulin, CD48, CD58, Protein Disulfide Isomerase Family A Member 3 (PDIA3/ERp57), and T Cell Receptor Alpha Constant polypeptides.

In another aspect, the invention of the disclosure features a method for producing a persistent allogeneic modified immune cell. The method involves (a) contacting a cell with a base editor containing a polynucleotide programmable DNA binding polypeptide (napDNAbp), a deaminase, and one or more guide RNAs (gRNAs) that target a nucleic acid molecule. The nucleic acid molecule encodes a polypeptide or contains a regulatory element associated with expression of the polypeptide. The polypeptide is selected from one or more of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP). The method further involves (b) overexpressing in the cell an inhibitory receptor, or fragment thereof, selected from one or more of Human Leukocyte Antigen-E (HLA-E), Human Leukocyte Antigen-G (HLA-G), Programmed Death Ligand 1 (PD-L1), and Cluster of Differentiation 47 (CD47).

In another aspect, the invention of the disclosure features an allogeneic modified immune cell produced according to the method of any of the above aspects, or embodiments thereof.

In another aspect, the invention of the disclosure provides an allogeneic modified immune cell containing a nucleobase alteration that reduces or eliminates expression of a polypeptide selected from one or more of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP).

In another aspect, the invention of the disclosure features a pharmaceutical composition containing an effective amount an allogeneic modified immune cell of any of the above aspects, or embodiments thereof.

In another aspect, the invention of the disclosure features a composition containing a guide RNA (gRNA) and a polynucleotide encoding a base editor containing a polynucleotide programmable DNA binding polypeptide (napDNAbp) domain and a deaminase domain. The gRNA contains a nucleic acid sequence that is complementary to a polynucleotide. The polynucleotide encodes a polypeptide or contains a regulatory element associated with expression of the polypeptide. The polypeptide is selected from one or more of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP).

In another aspect, the invention of the disclosure features a kit containing an allogeneic modified immune cell or composition of any of the above aspects, or embodiments thereof.

In another aspect, the invention of the disclosure features a method of treating cancer in a subject. The method involves administering to the subject an effective amount of an allogeneic modified immune cell of any of the above aspects, or embodiments thereof. In another aspect, the invention of the disclosure features a fusion polypeptide containing a loading peptide, at least a fragment of an HLA-G polypeptide, and at least a fragment of a β2M polypeptide.

In another aspect, the invention of the disclosure features a fusion polypeptide containing a loading peptide, at least a fragment of an HLA-E polypeptide, and at least a fragment of a β2M polypeptide.

In another aspect, the invention of the disclosure features a fusion polypeptide containing a loading peptide, and at least a fragment of an HLA-E polypeptide. In another aspect, the invention of the disclosure features a fusion polypeptide containing an amino acid sequence with at least 85% sequence identity to a sequence selected from one or more of:

HLA-G5+ IL-2 signal peptide
(SEQ ID NO: 1013)
MYRMQLLSCIALSLALVTNSGSHSMRYFSAAVSRPGRGEPRFIAMGYVDDTQFVRFDSDSACPR
MEPRAPWVEQEGPEYWEEETRNTKAHAQTDRMNLQTLRGYYNQSEASSHTLQWMIGCDLGSDGR
LLRGYEQYAYDGKDYLALNEDLRSWTAADTAAQISKRKCEAANVAEQRRAYLEGTCVEWLHRYL
ENGKEMLQRADPPKTHVTHHPVFDYEATLRCWALGFYPAEIILTWQRDGEDQTQDVELVETRPA
GDGTFQKWAAVVVPSGEEQRYTCHVQHEGLPEPLMLRWSKEGDGGIMSVRESRSLSEDL;
HLA-G5 Single chain trimer + IL-2 signal peptide
(SEQ ID NO: 1014)
MYRMQLLSCIALSLALVTNSIQRTPKIQVYSRHPAENGKSNFLNCYVSGFHPSDIEVDLLKNGE
RIEKVEHSDLSFSKDWSFYLLYYTEFTPTEKDEYACRVNHVTLSQPKIVKWDRDMGGGGSGGGG
SGGGGSRIIPRHLQLGGGGSGGGGSGGGGSGGGGSGSHSMRYFSAAVSRPGRGEPRFIAMGYVD
DTQFVRFDSDSACPRMEPRAPWVEQEGPEYWEEETRNTKAHAQTDRMNLQTLRGYYNQSEASSH
TLQWMIGCDLGSDGRLLRGYEQYAYDGKDYLALNEDLRSWTAADTAAQISKRKCEAANVAEQRR
AYLEGTCVEWLHRYLENGKEMLQRADPPKTHVTHHPVFDYEATLRCWALGFYPAEIILTWQRDG
EDQTQDVELVETRPAGDGTFQKWAAVVVPSGEEQRYTCHVQHEGLPEPLMLRWSKEGDGGIMSV
RESRSLSEDL;
HLA-E(ΔTM) Single chain trimer + HLA-G5 intron tail
(SEQ ID NO: 1015)
MSRSVALAVLALLSLSGLEAVMAPRTLFLGGGGSGGGGSGGGGSIQRTPKIQVYSRHPAENGKS
NFLNCYVSGFHPSDIEVDLLKNGERIEKVEHSDLSFSKDWSFYLLYYTEFTPTEKDEYACRVNH
VTLSQPKIVKWDRDMGGGGSGGGGSGGGGSGGGGSGSHSLKYFHTSVSRPGRGEPRFISVGYVD
DTQFVRFDNDAASPRMVPRAPWMEQEGSEYWDRETRSARDTAQIFRVNLRTLRGYYNQSEAGSH
TLQWMHGCELGPDGRFLRGYEQFAYDGKDYLTLNEDLRSWTAVDTAAQISEQKSNDASEAEHQR
AYLEDTCVEWLHKYLEKGKETLLHLEPPKTHVTHHPISDHEATLRCWALGFYPAEITLTWQQDG
EGHTQDTELVETRPAGDGTFQKWAAVVVPSGEEQRYTCHVQHEGLPEPVTLRWSKEGDGGIMSV
RESRSLSEDL;
HLA-E(ΔTM) β2M (C-term) Single chain trimer
(SEQ ID NO: 1016)
MSRSVALAVLALLSLSGLEAVMAPRTLFLGGSGGGASGGGSHSLKYFHTSVSRPGRGEPRFISV
GYVDDTQFVRFDNDAASPRMVPRAPWMEQEGSEYWDRETRSARDTAQIFRVNLRTLRGYYNQSE
AGSHTLQWMHGCELGPDGRFLRGYEQFAYDGKDYLTLNEDLRSWTAVDTAAQISEQKSNDASEA
EHQRAYLEDTCVEWLHKYLEKGKETLLHLEPPKTHVTHHPISDHEATLRCWALGFYPAEITLTW
QQDGEGHTQDTELVETRPAGDGTFQKWAAVVVPSGEEQRYTCHVQHEGLPEPVTLRWGGGGSGG
GGSGGGGSIQRTPKIQVYSRHPAENGKSNFLNCYVSGFHPSDIEVDLLKNGERIEKVEHSDLSF
SKDWSFYLLYYTEFTPTEKDEYACRVNHVTLSQPKIVKWDRDM;
HLA-E(ΔTM) Single chain dimer + HLA-G5 intron tail
(SEQ ID NO: 1017)
MSRSVALAVLALLSLSGLEAVMAPRTLFLGGSGGGASGGGSHSLKYFHTSVSRPGRGEPRFISV
GYVDDTQFVRFDNDAASPRMVPRAPWMEQEGSEYWDRETRSARDTAQIFRVNLRTLRGYYNQSE
AGSHTLQWMHGCELGPDGRFLRGYEQFAYDGKDYLTLNEDLRSWTAVDTAAQISEQKSNDASEA
EHQRAYLEDTCVEWLHKYLEKGKETLLHLEPPKTHVTHHPISDHEATLRCWALGFYPAEITLTW
QQDGEGHTQDTELVETRPAGDGTFQKWAAVVVPSGEEQRYTCHVQHEGLPEPVTLRWSKEGDGG
IMSVRESRSLSEDL;
and
HLA-E(ΔTM) Single chain dimer
(SEQ ID NO: 1018)
MSRSVALAVLALLSLSGLEAVMAPRTLFLGGSGGGASGGGSHSLKYFHTSVSRPGRGEPRFISV
GYVDDTQFVRFDNDAASPRMVPRAPWMEQEGSEYWDRETRSARDTAQIFRVNLRTLRGYYNQSE
AGSHTLQWMHGCELGPDGRFLRGYEQFAYDGKDYLTLNEDLRSWTAVDTAAQISEQKSNDASEA
EHQRAYLEDTCVEWLHKYLEKGKETLLHLEPPKTHVTHHPISDHEATLRCWALGFYPAEITLTW
QQDGEGHTQDTELVETRPAGDGTFQKWAAVVVPSGEEQRYTCHVQHEGLPEPVTLRW.

In another aspect, the invention of the disclosure features a membrane-bound fusion polypeptide. The fusion polypeptide contains a β2M domain and an HLA-E domain and/or a transmembrane domain.

In another aspect, the invention of the disclosure features a fusion polypeptide containing an amino acid sequence having at least 85% sequence identity to the following sequence:

(SEQ ID NO: 1019)
MSRSVALAVLALLSLSGLEAVMAPRTLFLGGGGSGGGGSGGGGSIQRTPK
IQVYSRHPAENGKSNFLNCYVSGFHPSDIEVDLLKNGERIEKVEHSDLSF
SKDWSFYLLYYTEFTPTEKDEYACRVNHVTLSQPKIVKWDRDMGGGGSGG
GGSGGGGSGGGGSGSHSLKYFHTSVSRPGRGEPRFISVGYVDDTQFVRFD
NDAASPRMVPRAPWMEQEGSEYWDRETRSARDTAQIFRVNLRTLRGYYNQ
SEAGSHTLQWMHGCELGPDGRFLRGYEQFAYDGKDYLTLNEDLRSWTAVD
TAAQISEQKSNDASEAEHQRAYLEDTCVEWLHKYLEKGKETLLHLEPPKT
HVTHHPISDHEATLRCWALGFYPAEITLTWQQDGEGHTQDTELVETRPAG
DGTFQKWAAVVVPSGEEQRYTCHVQHEGLPEPVTLRWKPASQPTIPIMAL
IVLGGVAGLLLFIGLGIFFCVRC.

In another aspect, the invention of the disclosure features a mammalian expression vector containing a polynucleotide sequence encoding the fusion polypeptide of any one of the above aspects, or embodiments thereof.

In another aspect, the invention of the disclosure features an allogeneic modified immune cell containing the vector of any of the above aspects, or embodiments thereof.

In another aspect, the invention of the disclosure features a method for producing a persistent allogeneic modified immune cell. The method involves contacting a cell with a polynucleotide programmable DNA binding polypeptide (napDNAbp) and one or more guide RNAs (gRNAs) that target the napDNAbp to cleave a target nucleic acid molecule and introduce an alteration in the target nucleic acid molecule, thereby producing a persistent allogeneic modified immune cell. The target nucleic acid molecule encodes a polypeptide and/or contains a regulatory element associated with expression thereof. The polypeptide is selected from one or more of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP). In any of the above aspects, or embodiments thereof, the method further involves contacting the cell with one or more guide RNAs that target the base editor to effect an alteration in a nucleic acid molecule. The nucleic acid molecule encodes a polypeptide and/or contains a regulatory element associated with expression thereof. The polypeptide is selected from one or more of beta-2 microglobulin, CD48, CD58, Protein Disulfide Isomerase Family A Member 3 (PDIA3/ERp57), and T Cell Receptor Alpha Constant polypeptides.

In any of the above aspects, or embodiments thereof, the method involves effecting a nucleobase alteration that reduces expression on the cell of one or more polypeptides selected from one or more of HLA-A, HLA-B, and HLA-C. In any of the above aspects, or embodiments thereof, the one or more gRNAs contain a nucleotide sequence with at least about 85% sequence identity to GCACUCACCCGCCCAGGUCU (SEQ ID NO: 817; TSBTx4190), GACCCGCAUCUCGGCGUCUG (SEQ ID NO: 827; TSBTx4200), CCUUACCCCAUCUCAGGGUG (SEQ ID NO: 820; TSBTx4193), and/or CUUACCCCAUCUCAGGGUGA (SEQ ID NO: 821; TSBTx4194). In any of the above aspects, or embodiments thereof, the method involves effecting a nucleobase alteration that reduces or eliminates expression on the cell of HLA-A and HLA-B, and the persistent allogeneic modified immune cell expresses HLA-C. In any of the above aspects, or embodiments thereof, the method involves effecting a nucleobase alteration that reduces or eliminates expression on the cell of HLA-A and HLA-B, and the persistent allogeneic modified immune cell expresses HLA-C and B2M.

In any of the above aspects, or embodiments thereof, the method further involves overexpressing in the cell an inhibitory receptor, or fragment thereof, selected from one or more of Human Leukocyte Antigen-E (HLA-E), Human Leukocyte Antigen-G (HLA-G), Programmed Death Ligand 1 (PD-L1), and Cluster of Differentiation 47 (CD47).

In any of the above aspects, or embodiments thereof, the method involves reducing or eliminating detectable expression on the cell of one or more polypeptides selected from one or more of HLA-A, HLA-B, and HLA-C relative to a corresponding unmodified cell.

In any of the above aspects, or embodiments thereof, the method reduces detectable expression of one or more of HLA-A, HLA-B, HLA-C, TAP1, TAP2, TAPBP, TAPBPL, NLRC5/CITA, CD155, MICA, and MICB by at least 25%.

In any of the above aspects, or embodiments thereof, the guide RNAs contain a nucleotide sequence selected from those listed in Tables 1A-1E or from SEQ ID NOs: 1214-2908, 403-412, and 435-446. In any of the above aspects, or embodiments thereof, the guide RNAs contain a spacer sequence selected from those listed in Tables 1A, 1B, and 1D. In any of the above aspects, or embodiments thereof, the guide RNA's comprise a gRNA sequence selected from those listed in Tables 1A, 1B, 1C, or 1E, or from SEQ ID NOs: 1214-2908, 403-412, and 435-446.

In any of the above aspects, or embodiments thereof, the deaminase is a cytidine deaminase and/or an adenosine deaminase. In embodiments, the adenosine deaminase is TadA or a TadA variant. In embodiments, the TadA is a TadA*8 or TadA*9. In embodiments, the cytidine deaminase is APOBEC or an APOBEC variant.

In any of the above aspects, or embodiments thereof, the base editor is rBE4 or ABE8.20m. In any of the above aspects, the base editor is ABE8.20m.

In any of the above aspects, or embodiments thereof, the base editor contains a complex containing the deaminase, the polynucleotide programmable DNA binding polypeptide (napDNAbp), and the guide RNA, or the base editor contains a fusion protein containing the polynucleotide programmable DNA binding polypeptide (napDNAbp) fused to the deaminase.

In any of the above aspects, or embodiments thereof, the method further involves contacting the cell with one or more guide RNAs that target the base editor to effect an alteration in a nucleic acid molecule encoding a polypeptide selected from one or more of TCRα Chain (TRAC), and Class II, Major Histocompatibility Complex Transactivator (CIITA).

In any of the above aspects, or embodiments thereof, the modified immune cell has increased persistence in a host, increased resistance to immune rejection, and/or decreased risk of eliciting a host-versus-graft reaction.

In any of the above aspects, or embodiments thereof, the napDNAbp is a Cas9 or a Cas12. In any of the above aspects, or embodiments thereof, the napDNAbp is a Cas12b. In any of the above aspects, or embodiments thereof, the napDNAbp is a Streptococcus pyogenes Cas9 (SpCas9), a Staphylococcus aureus Cas9 (SaCas9), Streptococcus thermophilus 1 Cas9 (St1Cas9), or variants thereof. In any of the above aspects, or embodiments thereof, the napDNAbp contains a nuclease dead Cas9 (dCas9) or a Cas9 nickase (nCas9).

In any of the above aspects, or embodiments thereof, the base editor further contains one or more uracil glycosylase inhibitors (UGIs).

In any of the above aspects, or embodiments thereof, the base editor further contains one or more nuclear localization signals (NLS). In embodiments, the NLS is a bipartite NLS.

In any of the above aspects, or embodiments thereof, the guide RNA contains a modification. In embodiments, the modification is a 2′-O-methyl 3′-phosphorothioate. In any of the above aspects, or embodiments thereof, the guide RNA contains modifications at the 3′ and 5′ termini.

In any of the above aspects, or embodiments thereof, the modified immune cell is a T cell, an NK cell, or a macrophage cell.

In any of the above aspects, or embodiments thereof, the alteration disrupts a splice acceptor or splice donor site or is in a promoter, intron, exon, enhancer, or an untranslated region (UTR). In any of the above aspects, or embodiments thereof, the alteration encodes a missense mutation and/or is associated with reduced expression of the polypeptide.

In any of the above aspects, or embodiments thereof, the method further involves expressing a chimeric antigen receptor (CAR) in the modified immune cell.

In any of the above aspects, or embodiments thereof, the cell contacted with the base editor is obtained from a healthy subject. In any of the above aspects, or embodiments thereof, the modified immune cell is derived from a cell obtained from a healthy subject.

In any of the above aspects, or embodiments thereof, the cell further contains a nucleobase alteration that reduces or eliminates expression of a polypeptide selected from one or more of beta-2 microglobulin, CD48, CD58, Protein Disulfide Isomerase Family A Member 3 (PDIA3/ERp57), and T Cell Receptor Alpha Constant polypeptides. In any of the above aspects, or embodiments thereof, the cell further contains a nucleobase alteration that reduces or eliminates expression of one or more polypeptides selected from one or more of HLA-A, HLA-B, and HLA-C. In any of the above aspects, or embodiments thereof, the cell overexpresses one or more inhibitory receptors selected from one or more of Human Leukocyte Antigen-E (HLA-E), Human Leukocyte Antigen-G (HLA-G), Programmed Death Ligand 1 (PD-L1), and Cluster of Differentiation 47 (CD47).

In any of the above aspects, or embodiments thereof, the modified immune cell further contains at least one alteration in a nucleic acid molecule encoding a polypeptide selected from one or more of TCRα Chain (TRAC), Cluster of Differentiation 58 (CD58), and Class II, Major Histocompatibility Complex Transactivator (CIITA).

In any of the above aspects, or embodiments thereof, the modified immune cell has reduced or inactivated surface HLA class-I expression, increased persistence in a host, increased resistance to immune rejection, and/or decreased risk of eliciting a host-versus-graft reaction relative to an unmodified reference immune cell. In any of the above aspects, or embodiments thereof, the allogeneic modified immune cell has increased persistence as compared to an unmodified reference immune cell when administered to a subject. In any of the above aspects, or embodiments thereof, persistence is increased by at least about 1 month. In any of the above aspects, or embodiments thereof, the allogeneic modified immune cell has increased T- and/or NK-cell resistance (i.e., increased resistance to T- and/or NK-cell mediated immune rejection) as compared to a reference immune cell when administered to a subject.

In any of the above aspects, or embodiments thereof, the allogeneic modified immune cell is a T cell, an NK cell, or a macrophage cell. In any of the above aspects, or embodiments thereof, the allogeneic modified immune cell expresses a chimeric antigen receptor (CAR).

In any of the above aspects, or embodiments thereof, the subject is a human subject.

In any of the above aspects, or embodiments thereof, the composition further contains a nucleic acid sequence that is complementary to a polynucleotide. The polynucleotide encodes a polypeptide or contains a regulatory element associated with expression of the polypeptide. The polypeptide is selected from one or more of beta-2 microglobulin, CD48, CD58, Protein Disulfide Isomerase Family A Member 3 (PDIA3/ERp57), and T Cell Receptor Alpha Constant polypeptides.

In any of the above aspects, or embodiments thereof, the composition contains a spacer selected from one or more of GCACUCACCCGCCCAGGUCU (SEQ ID NO: 817; TSBTx4190), GACCCGCAUCUCGGCGUCUG (SEQ ID NO: 827; TSBTx4200), CCUUACCCCAUCUCAGGGUG (SEQ ID NO: 820; TSBTx4193), and/or CUUACCCCAUCUCAGGGUGA (SEQ ID NO: 821; TSBTx4194).

In any of the above aspects, or embodiments thereof, the composition further contains a polynucleotide encoding an inhibitory receptor, or a fragment thereof, selected from one or more of Human Leukocyte Antigen-E (HLA-E), Human Leukocyte Antigen-G (HLA-G), Programmed Death Ligand 1 (PD-L1), and Cluster of Differentiation 47 (CD47).

In any of the above aspects, or embodiments thereof, the composition further contains a polynucleotide encoding a secreted or membrane-bound HLA-E and/or HLA-G single-chain trimer and/or single-chain dimer.

In any of the above aspects, or embodiments thereof, the composition further contains a polynucleotide encoding a polypeptide(s) with at least 85% sequence identity to an amino acid sequence listed in Table 19 and/or to the following amino acid sequence:

(SEQ ID NO: 1019)
MSRSVALAVLALLSLSGLEAVMAPRTLFLGGGGSGGGGSGGGGSIQRTPK
IQVYSRHPAENGKSNFLNCYVSGFHPSDIEVDLLKNGERIEKVEHSDLSF
SKDWSFYLLYYTEFTPTEKDEYACRVNHVTLSQPKIVKWDRDMGGGGSGG
GGSGGGGSGGGGSGSHSLKYFHTSVSRPGRGEPRFISVGYVDDTQFVRFD
NDAASPRMVPRAPWMEQEGSEYWDRETRSARDTAQIFRVNLRTLRGYYNQ
SEAGSHTLQWMHGCELGPDGRFLRGYEQFAYDGKDYLTLNEDLRSWTAVD
TAAQISEQKSNDASEAEHQRAYLEDTCVEWLHKYLEKGKETLLHLEPPKT
HVTHHPISDHEATLRCWALGFYPAEITLTWQQDGEGHTQDTELVETRPAG
DGTFQKWAAVVVPSGEEQRYTCHVQHEGLPEPVTLRWKPASQPTIPIMAL
IVLGGVAGLLLFIGLGIFFCVRC

In any of the above aspects, or embodiments thereof, the composition further contains a polynucleotide encoding a polypeptide selected from one or more of Human Leukocyte Antigen-E (HLA-E), Human Leukocyte Antigen-G (HLA-G), Programmed Death Ligand 1 (PD-L1), and Cluster of Differentiation 47 (CD47).

In any of the above aspects, or embodiments thereof, the gRNA contains a sequence selected from those listed in Tables 1A-1E or in the Sequence Listing as SEQ ID NOs: 1214-2908, 403-412, or 435-446.

In any of the above aspects, or embodiments thereof, the polynucleotide encoding the base editor contains mRNA.

In any of the above aspects, or embodiments thereof, the kit contains written instructions for using the allogeneic modified immune cell or the composition.

In any of the above aspects, or embodiments thereof, the modified immune cell has increased persistence in the subject, increased resistance to immune rejection, or decreased risk of eliciting a host-versus-graft reaction relative to a reference immune cell.

In any of the above aspects, or embodiments thereof, the allogeneic modified immune cell is a T cell or NK cell.

In any of the above aspects, or embodiments thereof, the reference immune cell expresses a CAR and normal levels of a major histocompatibility complex, class I polypeptide.

In any of the above aspects, or embodiments thereof, the recombinant polypeptide contains from N-terminus to C-terminus: a) a loading peptide, at least a fragment of an HLA-G polypeptide, and at least a fragment of a β2M polypeptide; b) at least a fragment of a β2M polypeptide, a loading peptide, and at least a fragment of an HLA-G polypeptide; c) a loading peptide, at least a fragment of a β2M polypeptide, and at least a fragment of an HLA-G polypeptide; or d) fragment of an HLA-G polypeptide, a loading peptide, and at least at least a fragment of a β2M polypeptide.

In any of the above aspects, or embodiments thereof, the recombinant polypeptide contains from N-terminus to C-terminus: a) a loading peptide, at least a fragment of an HLA-E polypeptide, and at least a fragment of a β2M polypeptide; b) at least a fragment of a β2M polypeptide, a loading peptide, and at least a fragment of an HLA-E polypeptide; c) a loading peptide, at least a fragment of a β2M polypeptide, and at least a fragment of an HLA-E polypeptide; or d) fragment of an HLA-E polypeptide, a loading peptide, and at least at least a fragment of a β2M polypeptide.

In any of the above aspects, or embodiments thereof, the recombinant polypeptide contains from N-terminus to C-terminus: a loading peptide, and at least a fragment of an HLA-E polypeptide. In any of the above aspects, or embodiments thereof, the HLA-G or HLA-E polypeptide lacks a transmembrane domain. In any of the above aspects, or embodiments thereof, the recombinant polypeptide further contains an HLA-G5 intron tail. In any of the above aspects, or embodiments thereof, the fusion polypeptide further contains one or more polypeptide linkers.

In any of the above aspects, or embodiments thereof, the recombinant polypeptide contains an N-terminal signal peptide. In any of the above aspects, or embodiments thereof, the transmembrane domain is an HLA-E transmembrane domain.

In any of the above aspects, or embodiments thereof, the method further involves providing one or more guide RNAs that target the napDNAbp to cleave a nucleic acid molecule. The nucleic acid molecule encodes a polypeptide and/or contains a regulatory element associated with expression thereof. The polypeptide is selected from one or more of beta-2 microglobulin, CD48, CD58, Protein Disulfide Isomerase Family A Member 3 (PDIA3/ERp57), and T Cell Receptor Alpha Constant polypeptides.

In any of the above aspects, or embodiments thereof, the modified immune cell has increased persistence in a host, increased resistance to immune rejection, decreased risk of eliciting a host-versus-graft reaction.

In any of the above aspects, or embodiments thereof, the napDNAbp further contains one or more nuclear localization signals (NLS). In any of the above aspects, or embodiments thereof, the cleavage disrupts a splice acceptor or splice donor site or is in a promoter, intron, exon, enhancer, or an untranslated region (UTR). In any of the above aspects, or embodiments thereof, the cleavage introduces a missense mutation and/or is associated with reduced expression of the polypeptide.

In any of the above aspects, or embodiments thereof, the alteration contains an insertion or a deletion.

In any of the above aspects, or embodiments thereof, modified immune cell further comprises virtually undetectable levels of one or more polypeptides selected from the group consisting of: B cell leukemia/lymphoma 11b (Bcl11b); B cell leukemia/lymphoma 2 related protein A1d (Bcl2a1d); B cell leukemia/lymphoma 6 (Bcl6); butyrophilin-like 6 (Btnl6); CD151 antigen (Cd151); chemokine (C—C motif) receptor 7 (Ccr7); discs large MAGUK scaffold protein 5 (Dlg5); erythropoietin (Epo); G protein-coupled receptor 18 (Gpr18); interferon alpha 15 (Ifnal5); interleukin 6 signal transducer (Il6st); interleukin 7 receptor (117r); Janus kinase 3 (Jak3); membrane associated ring-CH-type finger 7 (Marchf7); NCK associated protein 1 like (Nckap11); phospholipase A2, group IIF (Pla2g2f); runt related transcription factor 3 (Runx3); Signal-regulatory protein beta 1B (Sirpb1b); transforming growth factor, beta 1 (Tgfb1); tumor necrosis factor (ligand) superfamily, member 14 (Tnfsf14); tumor necrosis factor (ligand) superfamily, member 18 (Tnfsf18); tumor necrosis factor (ligand) superfamily, member 8 (Tnfsf8); zinc finger CCCH type containing 8 (Zc3h8); (Rac family small GTPase 2); (Slc4a1); 5-azacytidine induced gene 2 (Azi2); a disintegrin and metalloprotease domain 17 (Adam 17); a disintegrin and metalloprotease domain 8 (Adam8); Acetyl-CoA Acetyltransferase 1 (ACAT1); ACLY; adapter related protein complex 3 beta 1 sububit (Ap3b1); adapter related protein complex 3 delta 1 sububit (Ap3d1); adenosine A2a receptor (Adora2a); adenosine deaminase (Ada); adenosine kinase (Adk); adenosine regulating molecule 1 (Adrm1); advanced glycosylation end product-specific receptor (Ager) allograft inflammatory factor 1 (Aif1); AKT1; AKT2; amyloid beta (A4) precursor protein-binding family B member 1 interacting protein (Apbblip); ankyrin repeat and LEM domain (Ankle1); annecin A1 (Anxa1); arginase liver (Arg 1); arginase type II (Arg 2); AtPase Cu++ transporting, alpha polypeptide (Atp7a); autoimmune regulator (Aire); autophagy related 5 (Atg5); AXL; B and T Lymphocyte Associated (BTLA); B and T lymphocyte associated (Btla); B cell leukemia/lymphoma 10 (Bcl10); B cell leukemia/lymphoma 11a (Bcl11a); B cell leukemia/lymphoma 2 (Bcl2); B cell leukemia/lymphoma 3 (Bcl3); basic leucine zipper transcription factor, ATF-like (Batf); BCL2-associated X protein (Bax); BCL2L11; beta 2 microglobulin (B2m); BL2-associated agonist of cell dealth (Bad); BLIMP1; Bloom syndrome, RecQ like helicase (Blm); Bmi1 polycomb ring finger oncogene (Bmi1); Bone morphogenic protein 4 (Bmp4); Braf transforming gene (Braf); butyrophilin, subfamily 2, member A1 (Btn2a1); butyrophilin, subfamily 2, member A2 (Btn2a2); butyrophilin-like 1 (Btnl1); butyrophilin-like 2 (Btnl2); c-abl oncogene 1 (Abl1); c-abl oncogene 2 (Abl2); cadherin-like 26(Cdh26); calcium channel, voltage dependent, beta 4 subunit (Cacnb4); CAMK2D; capping protein regulator and myosin 1 linker 2 (Carmil2); carcinoembryonic antigen-related cell adhesion molecule (Ceacam1); Casitas B-lineage lymphoma b (Cblb); CASP8; Caspase 3 (Casp3); caspase recruitment domain family member 11 (Card11); catenin (cadherin associated protein), beta 1 (Ctnnb1); caveolin 1 (Cav1); CBL-B; CCAAT/enhancer binding protein (C/EBP), beta (Cebpb); CCR10; CCR4; CCR5; CCR6; CCR9; CD103; CD11a; CD122; CD123; CD127; CD130; CD132; CD160 antigen (Cd160); CD161; CD19; CD1d1 antigen (Cd1d1); CD1d2 antigen (CD1d2); CD2 antigen (CD2); CD209e antigen (Cd209e); CD23; CD244 molecule A (Cd244a); CD24a antigen (Cd24a); CD27 antigen (CD27); CD274 antigen (Cd274); CD276 antigen (Cd276); CD28 antigen (Cd28); CD3 delta; CD3 epsilon; CD3 gamma; CD30; CD300A molecule (Cd300a); CD33; CD38; CD4 antigen (Cd4); CD40 ligand (Cd40lg); CD44 antigen (Cd44); CD46 antigen, complement regulatory protein (Cd46); CD47 antigen (Rh-related antigen, integrin-associated signal transducer) (Cd47); CD48 antigen (Cd48); CD5 antigen (Cd5); CD52; CD58; CD59b antigen (Cd59b); CD6 antigen (Cd6); CD69; CD7; CD70; CD74 antigen (Cd74); CD8; CD8 antigen (Cd8); CD80 antigen (Cd80); CD81 antigen (Cd81); CD82; CD83 antigen (Cd83); CD86; CD86 antigen (Cd86); CD8A; CD96; CD99; CDK4; CDK8; CDKN1B; chemokine (C motif) ligand 1 (Xcl1); chemokine (C—C motif) ligand 19 (Ccl19); chemokine (C—C motif) ligand 2 (Ccl2); chemokine (C—C motif) ligand 20 (Ccl20); chemokine (C—C motif) ligand 5 (Ccl5); chemokine (C—C motif) receptor 2 (Ccr2); chemokine (C—C motif) receptor 6 (Ccr6); chemokine (C—C motif) receptor 9 (Ccr9); chemokine (C—X—C motif) ligand 12 (Cxcl12); chemokine (C—X—C motif) receptor (Cxcr4); Chitinase 3 Like 1 (Chi311); cholinergic receptor, nicotinic, alpha polypeptide 7 (Chrna7); chromodomain helicase DNA binding protein 7 (Chd7); CLA; Class II Major Histocompatibility Complex Transactivator (CIITA); cleft lip and palate associated transmembrane protein 1 (Clptm1); Cluster of Differentiation 123 (CD123); Cluster of Differentiation 3 (CD3); Cluster of Differentiation 33 (CD33); Cluster of Differentiation 52 (CD52); Cluster of Differentiation 7 (CD7); Cluster of Differentiation 96 (CD96); coagulation factor II (thrombin) receptor-like 1 (F2rl1); coil-coil domain containing 88B (Ccdc88b); core-binding factor beta (Cbfb); coronin, actin binding protein 1A (Coro1a); coxsackie virus and adenovirus receptor (Cxadr); CS-1; CSF2CSK; c-src tyrosine kinase (Csk); C-type lectin domain family 2, member i (Clec2i); C-type lectin domain family 4, member a2 (Clec4a2); C-type lectin domain family 4, member d (Clec4d); C-type lectin domain family 4, member e (Clec4e); C-type lectin domain family 4, member f (Clec4f); C-type lectin domain family 4, member g (Clec4g); CUL3; CXCR3; cyclic GMP-AMP synthase (Cgas); cyclin D3 (Ccnd3); cyclin dependent kinase inhibitor 2A (Cdkn2a); cyclin-dependent kinase (Cdk6); CYLD lysine 63 deubiquitinase (Cyld); cysteine-rich protein 3 (Crip3); cytidine 5′-triphosphate synthase (Ctps); Cytochrome P450 Family 11 Subfamily A Member 1 (Cyp11a1); cytochrome P450, family 26, subfamily b, polypeptide (Cyp26b1); Cytokine Inducible SH2 Containing Protein (CISH); cytotoxic T lymphocyte-associated protein 2 alpha (Ctla2a); Cytotoxic T-Lymphocyte Associated Protein 4 (CTLA-4); DCK; dedicator of cytokinesis 2 (Dock2); dedicator of cytokinesis 8 (Dock8); delta like canonical Notch ligand 4 (D114); deltex 1, E3 ubiquitin ligase (Dtx1); deoxyhypusine synthase (Dhps); DGKA; DGKZ; DHX37; dicer 1, ribonuclease type III (Dicer1); dipeptidylpeptidase 4 (Dpp4); discs large MAGUK scaffold protein 1 (Dlg1); DnaJ heat shock protein family (Hsp40) member A3 (Dnaja3); dolichyl-di-phosphooligosaccharide-protein glycotransferase (Ddost); double homeobox B-like 1 (Duxbl1); drosha, ribonuclease type III (Drosha); dual specificity phosphatase 10 (Dusp10); dual specificity phosphatase 22 (Dusp22); dual specificity phosphatase 3 (Dusp3); E74-like factor 4 (Elf4); early growth response 1 (Egr1); early growth response 3 (Egr3); ELOB (TCEB2); ENTPD1 (CD39); eomesodermin (Eomes); Eph receptor B4 (Ephb4); Eph receptor B6 (Ephb6); ephrin B1 (Efnbl); ephrin B2 (Efnb2); ephrin B3 (Efnb3); Epstein-Barr virus induced gene 3 (Ebi3); erb-b2 receptor tyrosine kinase (Erbb2); eukaryotic translation initiation factor 2 alpha kinase 4 (Eif2ak4); FADD; family with sequence similarity 49, member B (Fam49b); Fanconi anemia, complementation group A (Fanca); Fanconi anemia, complementation group D2 (Fancd2); Fas (TNF receptor superfamily member 6) (Fas); Fas (TNFRSF6)-associated via death domain (Fadd); Fas Cell Surface Death Receptor (FAS); Fc receptor, IgE, high affinity I, gamma polypeptide (Fcer1g); fibrinogen-like protein 1 (Fgl1); fibrinogen-like protein 2 (Fgl2); FK506 binding protein 1a (Fkbp1a); FK506 binding protein 1b ((Fkbp1b); flotillin 2 (Flot2); FMS-like tyrosine kinase (Flt3); forkhead box Ji (Foxj1); forkhead box N1 (Foxn1); forkhead box P1 (Foxp1); forkhead box P3 (Foxp3); frizzled class receptor 5 (Fzd5); frizzled class receptor 7 (Fzd7); frizzled class receptor 8 (Fzd8); fucosyltransferase 7 (Fut7); Fyn proto-oncogene (Fyn); gap junction protein, alpha 1 (Gja1); GATA binding protein 3 (GATA3); GCN2 kinase (IDO pathway); gelsolin (Gsn); GLI-Kruppel family member GLI3 (Gli3); glycerol-3-phosphate acyltransferase, mitochondrial (Gpam); growth arrest and DNA-damage-inducible 45 gamma (Gadd45g); GTPase, IMAP family member 1 (Gimap1); H1TET2; H2.0-like homeobox (Hlx); haematopoietic 1 (hem1); HCLS1 binding protein 3 (Hslbp3); heat shock 105 kDa/110 kDa protein 1 (Hsph1); heat shock protein 1 (chaperonin) (Hspd1); heat shock protein 90, alpha (cytosolic), class A member 1 (Hsp90aa1); hematopoietic SH2 domain containing (Hsh2d); hepatitis A virus cellular receptor 2 (Havcr2); hes family bHLH transcription factor 1 (Hes1); histocompatibility 2, class II antigen A, alpha (H2-Aa); histocompatibility 2, class II antigen A, beta 1 (H2-Abl); histocompatibility 2, class II, locus DMa (H2-DMa); histocompatibility 2, M region locus 3 (H3-M3); histocompatibility 2, O region alpha locus (H2-Oa); histocompatibility 2, T region locus 23 (H2-T23); HLA-DR; homeostatic iron regulator (Hfe); icos ligand (Icosl); IKAROS family zinc finger 1 (Ikzf1); IL10; IL10RA; IL2 inducible T cell kinase (Itk); IL6R; Indian hedgehog (Ihh); indoleamine 2,3-dioxygenase 1 (Idol); inducible T cell co-stimulator (Icos); inositol 1,4,5-trisphosphate 3-kinase B (Itpkb); insulin II (Ins2); insulin-like growth factor 1 (Igf1); insulin-like growth factor 2 (Igf2); insulin-like growth factor binding protein 2 (Igfbp2); integrin alpha L (Itgal); integrin alpha M (Itgam); integrin alpha V (Itgav); integrin alpha X (Itgax); integrin beta 2 (Itgb2); integrin, alpha D (Itgad); intercellular adhesion molecule 1 (Icam1); interferon (alpha and beta) receptor 1 (Ifnar1); interferon alpha 1 (Ifna1); interferon alpha 11 (Ifnal1); interferon alpha 12 (Ifnal2); interferon alpha 13 (Ifnal3); interferon alpha 14 (Ifnal4); interferon alpha 16 (Ifnal6); interferon alpha 2 (Ifna2); interferon alpha 4 (Ifna4); interferon alpha 5 (Ifna5); interferon alpha 6 (Ifna6); interferon alpha 7 (Ifna7); interferon alpha 9 (Ifna9); interferon alpha B (Ifnab); interferon beta 1 (Ifnb1); interferon gamma (Ifng); interferon kappa (Ifnk); interferon regulatory factor 1 (Irf1); interferon regulatory factor 4 (Irf4); interferon zeta (Ifnz); interleukin 1 beta (Il1b; interleukin 1 family, member 8 (Il1f8); interleukin 1 receptor-like 2 (Il1rl2); interleukin 12 receptor, beta1 (Il12rb1); interleukin 12a (1112a); interleukin 12b (1112b); interleukin 15 (I115); interleukin 18 (1118); interleukin 18 receptor 1 (I118r1); interleukin 2 (I12); interleukin 2 receptor, alpha chain (Il2ra); interleukin 2 receptor, gamma chain (Il2rg); interleukin 20 receptor beta (Il20rb); interleukin 21 (I121); interleukin 23, alpha subunit p19 (I123a); interleukin 27 (I127); interleukin 4 (I14); interleukin 4 receptor, alpha (Il4ra); interleukin 6 (I16); interleukin 7 (I17); IRF8; itchy, E3 ubiquitin protein ligase (Itch); jagged 2 (Jag2); jumonji domain containing 6 (Jmjd6); JUNB; junction adhesion molecule like 9 (Jam9); K(lysine) acetyltransferase 2A (Kat2a); KDEL (Lys-Asp-Glu-Leu) endoplasmic reticulum protein retention receptor 1 (Kdelr1); KIT proto-oncogene receptor tyrosine kinase (Kit); LAG-3; LAIR-1 (CD305); LDHA; lectin, galactose binding, soluble 1 (Lgals1); lectin, galactose binding, soluble 3 (Lgals3); lectin, galactose binding, soluble 8 (Lgals8); lectin, galactose binding, soluble 9 (Lgals9); leptin (Lep); leptin receptor (Lepr); leucine rich repeat containing 32 (Lrrc32); leukocyte immunoglobulin-like receptor, subfamily B, member 4A (Lilrb4a); LFNG O-fucosylpeptide 3-beta-N-acetylglucosaminyltransferase (Lfng); LIF; ligase IV, DNA, ATP-dependent (Lig4); LIM domain only 1 (Lmo1); limb region 1 like (Lmbrl); linker for activation of T cells (Lat); lymphocyte antigen 9 (Ly9); lymphocyte cytosolic protein 1 (Lcp1); lymphocyte protein tyrosine kinase (Lck); lymphocyte transmembrane adaptor 1 (Lax1); lymphocyte-activation gene 3 (Lag3); lymphoid enhancer binding factor 1 (Lef1); LYN; lysyl oxidase-like 3 (Loxl3); MAD1 mitotic arrest deficient 1-like 1 (Mad1l1); MALT1 paracaspase (Malt1); MAP4K4; MAPK14; MCJ; mechanistic target of rapamycin kinase (Mtor); MEF2D; Methylation-Controlled J Protein (MCJ); methyltransferase like 3 (Mettl3); MGAT5; MHC I like leukocyte 2 (Mill2); midkine (Mdk); mitogen-activated protein kinase 8 interacting protein 1 (Mapk8ip10); moesin (Msn); myelin protein zero-like 2 (Mpzl2); myeloblastosis oncogene (Myb); myosin, heavy polypeptide 9, non-muscle (Myh9); Nedd4 family interacting protein 1 (Ndfip1); neural precursor cell expressed, developmentally down-regulated 4 (Nedd4); NFATcT; NFATC2; NFATC4; NFKB activating protein (Nkap); nicastrin (Ncstn); NK2 homeobox 3 (Nkx2-3); NLR family, CARD domain containing 3 (Nlrc3); NLR family, pyrin domain containing 3 (Nlrp3); non-catalytic region of tyrosine kinase adaptor protein 1 (Nck1); non-catalytic region of tyrosine kinase adaptor protein 2 (Nck2); non-homologous end joining factor 1 (Nhej1); non-SMC condensin II complex, subunit H2 (Ncaph2); Notch-regulated ankyrin repeat protein (Nrarp); NT5E (CD73); nuclear factor of activated T cells, cytoplasmic, calcineurin dependent (Nfatc3); nuclear factor of kappa light polypeptide gene enhancer in B cells inhibitor, delta (Nfkbid); nuclear receptor co-repressor 1 (Ncor1); Nuclear Receptor Subfamily 4 Group A Member 1 (NR4A1); Nuclear Receptor Subfamily 4 Group A Member 2 (NR4A2); Nuclear Receptor Subfamily 4 Group A Member 3 (NR4A3); ODC1; OTU domain containing 5 (Otud5); OTULINL (FAM105A); paired box 1 (Pax1); PDCD1 (PD1; PD-1); PDIA3; pellino 1 (Peli1); peroxiredoxin 2 (Prdx2); PHD1 (EGLN2); PHD2 (EGLN1); PHD3 (EGLN3); phosphodiesterase 5A, cGMP-specific (Pde5a); phosphoinositide-3-kinase regulatory subunit (Pik3r6); phospholipase A2, group IIA (Pla2g2a); phospholipase A2, group IID (Pla2g2d); phospholipase A2, group IIE (Pla2g2e); phosphoprotein associated with glycosphingolipid microdomains 1 (Pag1); PIK3CD; PIKFYVE; POZ (BTB) and AT hook containing zinc finger 1 (Patz1); PPARa; PPARd; PR domain containing 1, with ZNF domain (Prdm1); presenilin 1 (Psen1); presenilin 2 (Psen2); PRKACA; PRKC, apoptosis, WT1, regulator (Pawr); programmed cell death 1 ligand 2 (Pdcd1lg2); prosaposin (Psap); prostaglandin E receptor 4 (subtype EP4) (Ptger4); protein kinase C, theta 2 (Prkcq); protein kinase C, zeta (Prkcz); protein kinase, cAMP dependent regulatory, type I, alpha (Prkar1a); protein kinase, DNA activated, catalytic polypeptide (Prkdc); protein phosphatase 3, catalytic subunit, beta isoform (Ppp3cb); protein tyrosine phosphatase, non-receptor type 2 (Ptpn2); protein tyrosine phosphatase, non-receptor type 22 (lymphoid) (Ptpn22); protein tyrosine phosphatase, non-receptor type 6 (Ptpn6); protein tyrosine phosphatase, receptor type, C (Ptprc); PTEN; PTPN11; purine-nucleoside phosphorylase (Pnp); purinergic receptor P2X, ligand-gated ion channel, 7 (P2rx7); PVR Related Immunoglobulin Domain Containing (PVRIG; CD112R); PYD and CARD domain containing 7 (Pycard); RAB27A, member RAS oncogene family (Rab27a); RAB29, member RAS oncogene family (Rab29); radical S-adenosyl methionine domain containing 2 (Rsad2); RAR-related orphan receptor alpha (Rora); RAR-related orphan receptor gamma (Ror); RAS guanyl releasing protein 1 (Rasgrpl); ras homolog family member A (Rhoa); ras homolog family member H (Rhoh); RAS protein activator like 3 (Rasal3); RASA2; receptor (TNFRSF)-interacting serine-threonine kinase 2 (Ripk2); recombination activating gene 1 (Rag1); recombination activating gene 2 (Rag2); Regulatory Factor X Associated Ankyrin Containing Protein (RFXANK); RHO family interacting cell polarization regulator 2 (Ripor2); ribosomal protein L22 (Rpl 22); ribosomal protein S6 (Rps6); RING CCCH (C3H) domains 1 (Rc3h1); ring finger and CCCH-type zinc finger domains 2 (Rc3h2); RNF2; runt related transcription factor 1 (Runx1); runt related transcription factor 2 (Runx2); SAM and SH3 domain containing 3 (Sash3); schlafen 1; Selectin P Ligand/P-Selectin Glycoprotein Ligand-1 (SELPG/PSGL1) polypeptide; selenoprotein K (Selenok); sema domain immunoglobulin domain (Ig), transmembrane domain (TM) and short cytoplasmic domain, (semaphorin) 4A (Sema4a); serine/threonine kinase 11 (Stk11); SH3 domain containing ring finger 1 (Sh3rf1); SHP1; sialophorin (Spn); SIGLEC15; signal transducer and activator of transcription 3 (Stat3); signal transducer and activator of transcription 5A (Stat5A); signal transducer and activator of transcription 5B (Stat5B); signal-regulatory protein alpha (Sirpa); Signal-regulatory protein beta 1A (Sirpb1a); Signal-regulatory protein beta 1C (Sirpb1c); SLA; SLAM family member 6 (Slamf6); SLAMF7; SMAD family member 3 (Smad3); SMAD family member 7 (Smad7); SMARCA4; solute carrier family 11 (proton-coupled divalent metal ion transporters), member 1 (Slc11a1); solute carrier family 4 (anion exchanger), member 1; solute carrier family 46, member 2 (Slc46a2); sonic hedgehog (Shh); SOS Ras/Rac guanine nucleotide exchange factor 1 (Sos1); SOS Ras/Rac guanine nucleotide exchange factor 2 (Sos2); special AT-rich sequence binding protein 1 (Satb1); spleen tyrosine kinase (Syk); Sprouty RTK Signaling Antagonist 1 (Spry1); Sprouty RTK Signaling Antagonist 2 (Spry2); squamous cell carcinoma antigen recognized by T cells (Sart1); src homology 2 domain-containing transforming protein B (Shb); Src-like-adaptor 2 (Sla2); SRY (sex determining region Y)-box 4 (Sox4); STK4; suppression inducing transmembrane adaptor 1 (Sit1); suppressor of cytokine signaling 1 (Socs1); suppressor of cytokine signaling 5 (Socs5); suppressor of cytokine signaling 6 (Socs6); surfactant associated protein D (Sftpd); SUV39; syndecan 4 (Sdc4); syntaxin 11 (Stxl 1); T Cell Immunoglobulin Mucin 3 (Tim-3); T cell immunoreceptor with Ig and ITIM domains (Tigit); T cell receptor alpha joining 18 (Traj18); T Cell Receptor Beta Constant 1 (TRBC1); T Cell Receptor Beta Constant 2 (TRBC2); T cell, immune regulator 1, ATPase, H+ transporting, lysosomal V0 protein A3 (Tcirg1); T cell-interacting, activating receptor on myeloid cells 1 (Tarm1); T-box 21 (Tbx21); TCR; TCR alpha; TCR beta; TCR complex gene sequence; Tet Methylcytosine Dioxygenase 2 (TET2); TGFbRII; TGFbRII (TGFBR2); three prime repair exonuclease 1 (Trex1); thymocyte selection associated (Themis); thymus cell antigen 1, theta (Thy 1); TMEM222; TNF receptor-associated factor 6 (Traf6); TNFAIP3; TNFRSF10B; TNFRSF8 (CD30); TOX; TOX2; TRAC; transformation related protein 53 (Trp53); Transforming Growth Factor Beta Receptor II (TGFbRII); transforming growth factor, beta receptor II (Tgfbr2); transmembrane 131 like (Tmeml311); transmembrane protein 98 (Tmem98); triggering receptor expressed on myeloid cells-like 2 (Treml2); TSC complex subunit 1 (Tsc1); tumor necrosis factor (ligand) superfamily, member 11 (Tnfsf11); tumor necrosis factor (ligand) superfamily, member 13b (Tnfsf13b); tumor necrosis factor (ligand) superfamily, member 4 (Tnfsf4); tumor necrosis factor (ligand) superfamily, member 9 (Tnfsf9); tumor necrosis factor receptor superfamily, member 13c (Tnfrsf13c); tumor necrosis factor receptor superfamily, member 4 (Tnfrsf4); tumor necrosis factor, alpha-induced protein 8-like 2 (Tnfalp812); twisted gastrulation BMP signaling modulator 1 (Twsg1); UBASH3A; vanin 1 (Vnn1); vascular cell adhesion molecule 1 (Vcam1); VHL; v-maf musculoaponeurotic fibrosarcoma oncogene family, protein B (avian) (Mafb); V-set and immunoglobulin domain containing 4 (Vsig4); V-Set Immunoregulatory Receptor (VISTA); WD repeat and FYVE domain containing 4 (Wdfy4); wingless-type MMTV integration site family, member 1 (Wnt1); wingless-type MMTV integration site family, member 4 (Wnt4); WNT signaling pathway regulator (Apc); WW domain containing E3 ubiquitin protein ligase 1 (Wwp1); XBP1; YAP1; ZAP70; ZC3H12A; zfp35; zinc finger and BTB domain containing 1 (Zbtb1); zinc finger and BTB domain containing 7B (Zbtb7B); zinc finger CCCH type containing 12A (Zc3h12a); zinc finger CCCH type containing 12D (Zc3h12d); zinc finger E-box binding homeobox 1 (Zeb1); zinc finger protein 36, C3H type (Zfp36); zinc finger protein 36, C3H type-like 1 (Zfp36L1); zinc finger protein 36, C3H type-like 2 (Zfp36L2); and zinc finger protein 683 (Zfp683).

In any of the above aspects, or embodiments thereof, the method further involves knocking out expression of HLA-A and HLA-B and does not comprise knocking out expression of B2M. In any of the above aspects, or embodiments thereof, the method further involves knocking out expression of HLA-A and HLA-B and does not comprise knocking out expression of B2M, CITA; NLRC5, TAP1, TAP2, ERp57 (PDIA3), and TAPBP (Tapasin). In any of the above aspects, or embodiments thereof, the method further involves knocking out expression of HLA-A and HLA-B and does not comprise knocking out expression of at least one, at least two, at least three, at least four, at least five, at least six, or all seven of B2M, CITA; NLRC5, TAP1, TAP2, ERp57 (PDIA3), and TAPBP (Tapasin).

In any of the above aspects, or embodiments thereof, the method further involves knocking out expression of HLA-A and HLA-B and does not comprise reducing (e.g., partially knocking out) expression of B2M. In any of the above aspects, or embodiments thereof, the method further involves knocking out expression of HLA-A and HLA-B and does not comprise reducing expression of B2M, CITA; NLRC5, TAP1, TAP2, ERp57 (PDIA3), and TAPBP (Tapasin). In any of the above aspects, or embodiments thereof, the method further involves knocking out expression of HLA-A and HLA-B and does not comprise reducing expression of at least one, at least two, at least three, at least four, at least five, at least six, or all seven of B2M, CITA; NLRC5, TAP1, TAP2, ERp57 (PDIA3), and TAPBP (Tapasin).

In any of the above aspects, or embodiments thereof, the method further involves knocking out expression of HLA-A and HLA-B and comprises reducing (e.g., partially knocking out) expression (e.g., by at least 25%, at least 50%, at least 75%, or more) of B2M. In any of the above aspects, or embodiments thereof, the method further involves knocking out expression of HLA-A and HLA-B and comprises reducing expression (e.g., by at least 25%, at least 50%, at least 75%, or more) of one or more of B2M, CITA; NLRC5, TAP1, TAP2, ERp57 (PDIA3), and TAPBP (Tapasin). In any of the above aspects, or embodiments thereof, the method further involves knocking out expression of HLA-A and HLA-B and comprises reducing expression (e.g., by at least 25%, at least 50%, at least 75%, or more) of at least one, at least two, at least three, at least four, at least five, at least six, or all seven of B2M, CITA; NLRC5, TAP1, TAP2, ERp57 (PDIA3), and TAPBP (Tapasin).

In any of the above aspects, or embodiments thereof, the method further involves contacting the cell with one or more guide RNAs that target the base editor to effect an alteration in a nucleic acid molecule, where the nucleic acid molecule encodes a polypeptide and/or contains a regulatory element associated with expression thereof. The polypeptide is selected from one or more of CD155, Nectin-2, CD48, MICA, MICB, and ULBP.

### Definitions

Unless defined otherwise, all technical and scientific terms used herein have the meaning commonly understood by a person skilled in the art to which this invention belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “cluster of differentiation 155 (CD155) polypeptide,” also termed the Poliovirus Receptor (PVR), is meant a protein having at least about 85% amino acid sequence identity to GenBank Accession No. AAF69803.1, which is provided below, or a fragment thereof having cell adhesion and/or immunomodulatory activity.

(SEQ ID NO: 1020)
>AAF69803.1 poliovirus receptor [Homo sapiens]
MARAMAAAWPLLLVALLVLSWPPPGTGDVVVQAPTQVPGFLGDSVTLPCY
LQVPNMEVTHVSQLTWARHGESGSMAVFHQTQGPSYSESKRLEFVAARLG
AELRNASLRMFGLRVEDEGNYTCLFVTFPQGSRSVDIWLRVLAKPQNTAE
VQKVQLTGEPVPMARCVSTGGRPPAQITWHSDLGGMPNTSQVPGFLSGTV
TVTSLWILVPSSQVDGKNVTCKVEHESFEKPQLLTVNLTVYYPPEVSISG
YDNNWYLGQNEATLTCDARSNPEPTGYNWSTTMGPLPPFAVAQGAQLLIR
PVDKPINTTLICNVTNALGARQAELTVQVKEGPPSEHSGISRNAIIFLVL
GILVFLILLGIGIYFYWSKCSREVLWHCHLCPSSTEHASASANGHVSYSA
VSRENSSSQDPQTEGTR

By “cluster of differentiation 155 (CD155) polynucleotide” is meant a nucleic acid molecule encoding an CD155 polypeptide, as well as the introns, exons, 3′ untranslated regions, 5′ untranslated regions, and regulatory sequences associated with its expression, or fragments thereof. An exemplary CD155 polynucleotide sequence is provided at Genbank Accession No. AC068948.1, which is provided below.

>AC068948.1:12735-12813, 15833-16180, 18419-18715,
22507-22624, 26368-26516, 27348-27506, 29897-29928,
30345-30416 Homo sapiens chromosome 19, cosmid R28371
(LLNL-R_244D7), complete sequence
(SEQ ID NO: 1021)
ATGGCCCGAGCCATGGCCGCCGCGTGGCCGCTGCTGCTGGTGGCGCTACTGGTGCTGTCCTGGC
CACCCCCAGGAACCGGGGACGTCGTCGTGCAGGCGCCCACCCAGGTGCCCGGCTTCTTGGGCGA
CTCCGTGACGCTGCCCTGCTACCTACAGGTGCCCAACATGGAGGTGACGCATGTGTCACAGCTG
ACTTGGGCGCGGCATGGTGAATCTGGCAGCATGGCCGTCTTCCACCAAACGCAGGGCCCCAGCT
ATTCGGAGTCCAAACGGCTGGAATTCGTGGCAGCCAGACTGGGCGCGGAGCTGCGGAATGCCTC
GCTGAGGATGTTCGGGTTGCGCGTAGAGGATGAAGGCAACTACACCTGCCTGTTCGTCACGTTC
CCGCAGGGCAGCAGGAGCGTGGATATCTGGCTCCGAGTGCTTGCCAAGCCCCAGAACACAGCTG
AGGTTCAGAAGGTCCAGCTCACTGGAGAGCCAGTGCCCATGGCCCGCTGCGTCTCCACAGGGGG
TCGCCCGCCAGCCCAAATCACCTGGCACTCAGACCTGGGCGGGATGCCCAATACGAGCCAGGTG
CCAGGGTTCCTGTCTGGCACAGTCACTGTCACCAGCCTCTGGATATTGGTGCCCTCAAGCCAGG
TGGACGGCAAGAATGTGACCTGCAAGGTGGAGCACGAGAGCTTTGAGAAGCCTCAGCTGCTGAC
TGTGAACCTCACCGTGTACTACCCCCCAGAGGTATCCATCTCTGGCTATGATAACAACTGGTAC
CTTGGCCAGAATGAGGCCACCCTGACCTGCGATGCTCGCAGCAACCCAGAGCCCACAGGCTATA
ATTGGAGCACGACCATGGGTCCCCTGCCACCCTTTGCTGTGGCCCAGGGCGCCCAGCTCCTGAT
CCGTCCTGTGGACAAACCAATCAACACAACTTTAATCTGCAACGTCACCAATGCCCTAGGAGCT
CGCCAGGCAGAACTGACCGTCCAGGTCAAAGAGGGACCTCCCAGTGAGCACTCAGGCATATCCC
GTAACGCCATCATCTTCCTGGTTCTGGGAATCCTGGTTTTTCTGATCCTGCTGGGGATCGGGAT
TTATTTCTATTGGTCCAAATGTTCCCGTGAGGTCCTTTGGCACTGTCATCTGTGTCCCTCGAGT
ACAGAGCATGCCAGCGCCTCAGCTAATGGGCATGTCTCCTATTCAGCTGTGAGCAGAGAGAACA
GCTCTTCCCAGGATCCACAGACAGAGGGCACAAGGTGA.
The CD155 gene corresponds to ENSG00000073008.15.

By “cluster of differentiation 48 (CD48) polypeptide” is meant a protein having at least about 85% amino acid sequence identity to any one of GenBank Accession Nos. NP_001242959, NP_001769, and AAA62834.1, which is provided below, or a fragment thereof having immunomodulatory activity.

>NP_001242959.1 CD48 antigen isoform 2 precursor [Homo sapiens]
(SEQ ID NO: 1046)
MCSRGWDSCLALELLLLPLSLLVTSQGHLVHMTVVSGSNVTLNISESLPENYKQLTWFYTFDQ
KIVEWDSRKSKYFESKFKGRVRLDPQSGALYISKVQKEDNSTYIMRVLKKTGNEQEWKIKLQVL
DPVPKPVIKIEKIEDMDDNCYLKLSCVIPGESVNYTWYGDKRPFPKELQNSVLETTLMPHNYSR
CYTCQVSNSVSSKNGTVCLSPPCTLGKKDPWELRGAQGNWSCFEQRKAGGPIQPPCTVWW.
>NP_001769.2 CD48 antigen isoform 1 precursor [Homo sapiens]
(SEQ ID NO: 0147)
MCSRGWDSCLALELLLLPLSLLVTSIQGHLVHMTVVSGSNVTLNISESLPENYKQLTWFYTFDQ
KIVEWDSRKSKYFESKFKGRVRLDPQSGALYISKVQKEDNSTYIMRVLKKTGNEQEWKI KLQVL
DPVPKPVIKIEKIEDMDDNCYLKLSCVIPGESVNYTWYGDKRPFPKELQNSVLETTLMPHNYSR
CYTCQVSNSVSSKNGTVCLSPPCTLARSFGVEWIASWLVVTVPTILGLLLT.
>AAA62834.1 pan-leukocyte antigen [Homo sapiens]
(SEQ ID NO: 1022)
MWSRGWDSCLALELLLLPLSLLVTSIQGHLVHMTVVSGSNVTLNISESLPENYKQLTWFYTFDQ
KIVEWDSRKSKYFESKFKGRVRLDPQSGALYISKVQKEDNSTYIMRVLKKTGNEQEWKIKLQVL
DPVPKPVIKIEKIEDMDDNCYLKLSCVIPGESVNYTWYGDKRPFPKELQNSVLETTLMPHNYSR
CYTCQVSNSVSSKNGTVCLSPPCTLARSFGVEWIASWLVVTVPTILGLLLT.

By “cluster of differentiation 48 (CD48) polynucleotide” is meant a nucleic acid molecule encoding an CD48 polypeptide, as well as the introns, exons, 3′ untranslated regions, 5′ untranslated regions, and regulatory sequences associated with its expression, or fragments thereof. One exemplary CD48 polynucleotide sequence is provided at Genbank Accession No. M59904.1, which is provided below.

>M59904.1:18-749 Human pan-leukocyte antigen (CD48) mRNA,
complete cds
(SEQ ID NO: 1023)
ATGTGGTCCAGAGGTTGGGATTCGTGTCTGGCTCTGGAATTGCTACTGCTGCCTCTGTCACTCC
TGGTGACCAGCATTCAAGGTCACTTGGTACATATGACCGTGGTCTCCGGCAGCAACGTGACTCT
GAACATCTCTGAGAGCCTGCCTGAGAACTACAAACAACTAACCTGGTTTTATACTTTCGACCAG
AAGATTGTAGAATGGGATTCCAGAAAATCTAAGTACTTTGAATCCAAATTTAAAGGCAGGGTCA
GACTTGATCCTCAGAGTGGCGCACTGTACATCTCTAAGGTCCAGAAAGAGGACAACAGCACCTA
CATCATGAGGGTGTTGAAAAAGACTGGGAATGAGCAAGAATGGAAGATCAAGCTGCAAGTGCTT
GACCCTGTACCCAAGCCTGTCATCAAAATTGAGAAGATAGAAGACATGGATGACAACTGTTATC
TGAAACTGTCATGTGTGATACCTGGCGAGTCTGTAAACTACACCTGGTATGGGGACAAAAGGCC
CTTCCCAAAGGAGCTCCAGAACAGTGTGCTTGAAACCACCCTTATGCCACATAATTACTCCAGG
TGTTATACTTGCCAAGTCAGCAATTCTGTGAGCAGCAAGAATGGCACCGTCTGCCTCAGTCCAC
CCTGTACCCTGGCCCGGTCCTTTGGAGTAGAATGGATTGCAAGTTGGCTAGTGGTCACGGTGCC
CACCATTCTTGGCCTGTTACTTACCTGA

Other exemplary sequences are provided below:

>NM_001256030.2 Homo sapiens CD48 molecule (CD48),
transcript variant 2, mRNA
(SEQ ID NO: 1048)
CTTTTTCTAGCCAGGCTCTCAACTGTCTCCTGCGTTGCTGGGAAGTTCTGGAAGGAAGCATGTG
CTCCAGAGGTTGGGATTCGTGTCTGGCTCTGGAATTGCTACTGCTGCCTCTGTCACTCCTGGTG
ACCAGCATTCAAGGTCACTTGGTACATATGACCGTGGTCTCCGGCAGCAACGTGACTCTGAACA
TCTCTGAGAGCCTGCCTGAGAACTACAAACAACTAACCTGGTTTTATACTTTCGACCAGAAGAT
TGTAGAATGGGATTCCAGAAAATCTAAGTACTTTGAATCCAAATTTAAAGGCAGGGTCAGACTT
GATCCTCAGAGTGGCGCACTGTACATCTCTAAGGTCCAGAAAGAGGACAACAGCACCTACATCA
TGAGGGTGTTGAAAAAGACTGGGAATGAGCAAGAATGGAAGATCAAGCTGCAAGTGCTTGACCC
TGTACCCAAGCCTGTCATCAAAATTGAGAAGATAGAAGACATGGATGACAACTGTTATCTGAAA
CTGTCATGTGTGATACCTGGCGAGTCTGTAAACTACACCTGGTATGGGGACAAAAGGCCCTTCC
CAAAGGAGCTCCAGAACAGTGTGCTTGAAACCACCCTTATGCCACATAATTACTCCAGGTGTTA
TACTTGCCAAGTCAGCAATTCTGTGAGCAGCAAGAATGGCACGGTCTGCCTCAGTCCACCCTGT
ACCCTGGGTAAGAAGGATCCCTGGGAGCTGAGGGGGGCACAGGGTAACTGGAGTTGTTTTGAAC
AAAGAAAGGCTGGGGGTCCTATTCAGCCTCCTTGCACAGTGTGGTGGTGAATCCCTAAGGTGTC
TGGGAGAGCTGGGAGACGTGGGTTCTGCCACCAGCTCTACCACCACCTCCCAGCCAGCTTACCT
CAACTTCGTGGGGGCTCAGTGTTCTCACCTGCAAAGGACGTTTGGGAGAGATCTCTGATACTCC
TCTTCCCTCTCCCGCTCTAACAAAGCATAGTCCTAACATCTGAGGCCAGGGTCATCATAGAGTA
GACTGAAACATCAGGGTGAGCAGGGAGAAGGAAGGGCAAGTGGGCGAGCAGCTGTCTAGAGGGG
CTTCATTAGACAGCCGAAGTCAGCCAAGGAAAGAGGGACCGAGGTCATTAGACCGCCAAAGTCA
GCCAGGGAAAGAGGGACTGAGGAGACGGGCCTGAGAGAGGCCGTCGAGGAGGCGTGAGAGCCTG
AGCCTCAGGCGAAGCTTCTCCTCCCCAGCCTGATGTTCCTAGATGAACTTAGGAAGCCAGATTC
CCCTGTCTCCTGGGAGGATCCACTCATGAGTGTCACACCTGGCTCTAGATCAGGCCTACACTGG
TGCTAGCATGGGACAGCTAAGGCCATGGGTTTTAGAGTCAGTCATACCTGGGGTCACTTCTAGG
ACTGTCACTTACTAGCTAAACAAGTTACTTAGCTTCCCCAAGTCATGTTCTTCCTAAATAAAGG
ACAAAATAACAGTT.
>NM_001778.4 Homo sapiens CD48 molecule (CD48),
transcript variant 1, mRNA
(SEQ ID NO: 1049)
CTTTTTCTAGCCAGGCTCTCAACTGTCTCCTGCGTTGCTGGGAAGTTCTGGAAGGAAGCATGTG
CTCCAGAGGTTGGGATTCGTGTCTGGCTCTGGAATTGCTACTGCTGCCTCTGTCACTCCTGGTG
ACCAGCATTCAAGGTCACTTGGTACATATGACCGTGGTCTCCGGCAGCAACGTGACTCTGAACA
TCTCTGAGAGCCTGCCTGAGAACTACAAACAACTAACCTGGTTTTATACTTTCGACCAGAAGAT
TGTAGAATGGGATTCCAGAAAATCTAAGTACTTTGAATCCAAATTTAAAGGCAGGGTCAGACTT
GATCCTCAGAGTGGCGCACTGTACATCTCTAAGGTCCAGAAAGAGGACAACAGCACCTACATCA
TGAGGGTGTTGAAAAAGACTGGGAATGAGCAAGAATGGAAGATCAAGCTGCAAGTGCTTGACCC
TGTACCCAAGCCTGTCATCAAAATTGAGAAGATAGAAGACATGGATGACAACTGTTATCTGAAA
CTGTCATGTGTGATACCTGGCGAGTCTGTAAACTACACCTGGTATGGGGACAAAAGGCCCTTCC
CAAAGGAGCTCCAGAACAGTGTGCTTGAAACCACCCTTATGCCACATAATTACTCCAGGTGTTA
TACTTGCCAAGTCAGCAATTCTGTGAGCAGCAAGAATGGCACGGTCTGCCTCAGTCCACCCTGT
ACCCTGGCCCGGTCCTTTGGAGTAGAATGGATTGCAAGTTGGCTAGTGGTCACGGTGCCCACCA
TTCTTGGCCTGTTACTTACCTGAGATGAGCTCTTTTAACTCAAGCGAAACTTCAAGGCCAGAAG
ATCTTGCCTGTTGGTGATCATGCTCCTCACCAGGACAGAGACTGTATAGGCTGACCAGAAGCAT
GCTGCTGAATTATCAACGAGGATTTTCAAGTTAACTTTTAAATACTGGTTATTATTTAATTTTA
TATCCCTTTGTTGTTTTCTAGTACACAGAGATATAGAGATACACATGCTTTTTTCCCACCCAAA
ATTGTGACAACATTATGTGAATGTTTTATTATTTTTTAAAATAAACATTTGATATAATTGTCAA
TTAACTGAA.
The gene for CD48 corresponds to Ensemble Accession No.
ENSG00000117091.

By “major histocompatibility complex, class I, A (HLA-A) polypeptide” is meant a protein having at least about 60%, 70%, or 85% amino acid sequence identity to GenBank Accession No. BAA07530.1, which is provided below, or a fragment thereof having antigen presenting activity.

>BAA07530.1 HLA-A [Homo sapiens]
(SEQ ID NO: 1024)
MAVMAPRTLVLLLSGALALTQTWAGSHSMRYFFTSVSRPGRGEPRFIAVG
YVDDTQFVRFDSDAASQRMEPRAPWIEQEGPEYWDGETRKVKAHSQTHRV
DLGTLRGYYNQSEAGSHTVQRMCGCDVGSDWRFLRGYHQYAYDGKDYIAL
KEDLRSWTAADMAAQTTKHKWEAAHVAEQLRAYLEGTCVEWLRRYLENGK
ETLQRTDAPKTHMTHHAVSDHEATLRCWALSFYPAEITLTWQRDGEDQTQ
DTELVETRPAGDGTFQKWAAVVVPSGQEQR

By “major histocompatibility complex, class I, A (HLA-A) polynucleotide” is meant a nucleic acid molecule encoding an HLA-A polypeptide, as well as the introns, exons, 3′ untranslated regions, 5′ untranslated regions, and regulatory sequences associated with its expression, or fragments thereof. An exemplary HLA-A polynucleotide sequence is provided at Genbank Accession No. D38525.1, which is provided below.

>D38525.1:1-843 Homo sapiens HLA-A mRNA, complete cds, HLA-A null
allele HLA-A*0215N
(SEQ ID NO: 1025)
ATGGCCGTCATGGCGCCCCGAACCCTCGTCCTGCTACTCTCGGGGGCTCTGGCCCTGACCCAGA
CCTGGGCGGGCTCTCACTCCATGAGGTATTTCTTCACATCCGTGTCCCGGCCCGGCCGCGGGGA
GCCCCGCTTCATCGCAGTGGGCTACGTGGACGACACGCAGTTCGTGCGGTTCGACAGCGACGCC
GCGAGCCAGAGGATGGAGCCGCGGGCGCCGTGGATAGAGCAGGAGGGTCCGGAGTATTGGGACG
GGGAGACACGGAAAGTGAAGGCCCACTCACAGACTCACCGAGTGGACCTGGGGACCCTGCGCGG
CTACTACAACCAGAGCGAGGCCGGTTCTCACACCGTCCAGAGGATGTGTGGCTGCGACGTGGGG
TCGGACTGGCGCTTCCTCCGCGGGTACCACCAGTACGCCTACGACGGCAAGGATTACATCGCCC
TGAAAGAGGACCTGCGCTCTTGGACCGCGGCGGACATGGCAGCTCAGACCACCAAGCACAAGTG
GGAGGCGGCCCATGTGGCGGAGCAGTTGAGAGCCTACCTGGAGGGCACGTGCGTGGAGTGGCTC
CGCAGATACCTGGAGAACGGGAAGGAGACGCTGCAGCGCACGGACGCCCCCAAAACGCATATGA
CTCACCACGCTGTCTCTGACCATGAAGCCACCCTGAGGTGCTGGGCCCTGAGCTTCTACCCTGC
GGAGATCACACTGACCTGGCAGCGGGATGGGGAGGACCAGACCCAGGACACGGAGCTCGTGGAG
ACCAGGCCTGCAGGGGATGGAACCTTCCAGAAGTGGGCGGCTGTGGTGGTGCCTTCTGGACAGG
AGCAGAGATAA.
The HLA-A gene corresponds to Ensemble ENSG00000206503.

By “major histocompatibility complex, class I, B (HLA-B) polypeptide” is meant a protein having at least about 85% amino acid sequence identity to GenBank Accession No. CAD30340.1, which is provided below, or a fragment thereof having antigen presenting activity.

>CAD30340.1 HLA-B [Homo sapiens]
(SEQ ID NO: 1026)
MRVTAPRTVLLLLSAALALTETWAGSHSMRYFHTAMSRPGRGEPRFITV
GYVDDTLFVRFDSDATSPRKEPRAPWIEQEGPEYWDRETQISKTNTQTY
RESLRNLRGYYNQSEAGSHTWQRMYGCDLGPDGRLLRGYNQLAYDGKDY
IALNEDLSSWTAADTAAQITQRKWEAARVAEQDRAYLEGLCVESLRRYL
ENGKETLQRADPPKTHVTHHPISDHEATLRCWALGFYPAEITLTWQRDG
EDQTQDTELVETRPAGDRTFQKWAAVVVPSGEEQRYTCHVQHEGLPKPLT
LRWEPSSQSTIPIVGIVAGLAVLAVVVIGAVVATVMCRRKSSGGKGGSYS
QAASSDSAQGSDVSLTA

By “major histocompatibility complex, class I, B (HLA-B) polynucleotide” is meant a nucleic acid molecule encoding an HLA-B polypeptide, as well as the introns, exons, 3′ untranslated regions, 5′ untranslated regions, and regulatory sequences associated with its expression, or fragments thereof. An exemplary HLA-B polynucleotide sequence is provided at Genbank Accession No. AJ458992.1, which is provided below.

>AJ458992.1:302-374, 503-772 ,1023-1298, 1872-2147, 2252-2368,
2810-2842, 2949-2992 Homo sapiens HLA-B gene for MHC class I
antigen, HLA-B*4501 allele, exons 1-7
(SEQ ID NO: 1027)
ATGCGGGTCACGGCACCCCGAACCGTCCTCCTGCTGCTCTCGGCGGCCCTGGCCCTGACCGAGA
CCTGGGCCGGCTCCCACTCCATGAGGTATTTCCACACCGCCATGTCCCGGCCCGGCCGCGGGGA
GCCCCGCTTCATCACCGTGGGCTACGTGGACGACACGCTGTTCGTGAGGTTCGACAGCGACGCC
ACGAGTCCGAGGAAGGAGCCGCGGGCGCCATGGATAGAGCAGGAGGGGCCGGAGTATTGGGACC
GGGAGACACAGATCTCCAAGACCAACACACAGACTTACCGAGAGAGCCTGCGGAACCTGCGCGG
CTACTACAACCAGAGCGAGGCCGGGTCTCACACTTGGCAGAGGATGTATGGCTGCGACCTGGGG
CCCGACGGGCGCCTCCTCCGCGGGTATAACCAGTTAGCCTACGACGGCAAGGATTACATCGCCC
TGAACGAGGACCTGAGCTCCTGGACCGCGGCGGACACCGCGGCTCAGATCACCCAGCGCAAGTG
GGAGGCGGCCCGTGTGGCGGAGCAGGACAGAGCCTACCTGGAGGGCCTGTGCGTGGAGTCGCTC
CGCAGATACCTGGAGAACGGGAAGGAGACGCTGCAGCGCGCGGACCCCCCAAAGACACATGTGA
CCCACCACCCCATCTCTGACCATGAGGCCACCCTGAGGTGCTGGGCCCTGGGCTTCTACCCTGC
GGAGATCACACTGACCTGGCAGCGGGATGGCGAGGACCAAACTCAGGACACCGAGCTTGTGGAG
ACCAGACCAGCAGGAGATAGAACCTTCCAGAAGTGGGCAGCTGTGGTGGTGCCTTCTGGAGAAG
AGCAGAGATACACATGCCATGTACAGCATGAGGGGCTGCCGAAGCCCCTCACCCTGAGATGGGA
GCCATCTTCCCAGTCCACCATCCCCATCGTGGGCATTGTTGCTGGCCTGGCTGTCCTAGCAGTT
GTGGTCATCGGAGCTGTGGTCGCTACTGTGATGTGTAGGAGGAAGAGCTCAGGTGGAAAAGGAG
GGAGCTACTCTCAGGCTGCGTCCAGCGACAGTGCCCAGGGCTCTGATGTGTCTCTCACAGCTTG
A.

By “major histocompatibility complex, class I, C (HLA-C) polypeptide” is meant a protein having at least about 85% amino acid sequence identity to GenBank Accession No. BB094058.1, which is provided below, or a fragment thereof having antigen presenting activity.

>BBO94058.1 MHC class I antigen, HLA-C alpha chain [Homo sapiens]
(SEQ ID NO: 1028)
MRVMAPRTLILLLSGALALTETWACSHSMRYFYTAVSRPGRGEPRFIAVGYVDDTQFVRFDSDA
ASPRGEPRAPWVEQEGPEYWDRETQKYKRQAQADRVSLRNLRGYYNQSEAGSHTLQRMYGCDLG
PDGRLLRGYDQSAYDGKDYIALNEDLRSWTAADTAAQITQRKWEAAREAEQWRAYLEGTCVEWL
RRYLENGKETLQRAEHPKTHVTHHPVSDHEATLRCWALGFYPAEITLTWQRDGEDQTQDTELVE
TRPAGDGTFQKWAAVVVPSGEEQRYTCHVQHEGLPEPLTLRWEPSSQPTIPIMGIVAGLAVLAV
LAVLGAVMAVVMCRRKSSGGKGGSCSQAASSNSAQGSDESLIACKA

By “major histocompatibility complex, class I, C (HLA-C) polynucleotide” is meant a nucleic acid molecule encoding an HLA-C polypeptide, as well as the introns, exons, 3′ untranslated regions, 5′ untranslated regions, and regulatory sequences associated with its expression, or fragments thereof. An exemplary HLA-C polynucleotide sequence is provided at Genbank Accession No. LC508210.1, which is provided below.

>LC508210.1:1-73, 204-473, 720-995, 1583-1858, 1980-2099, 2539-
2571, 2679-2726, 2891-2895 Homo sapiens HLA-C gene for MHC class I
antigen, HLA-C alpha chain, complete cds, HLA-C*12:02:02:01 variant
(SEQ ID NO: 1029)
ATGCGGGTCATGGCGCCCCGAACCCTCATCCTGCTGCTCTCGGGAGCCCTGGCCCTGACCGAGA
CCTGGGCCTGCTCCCACTCCATGAGGTATTTCTACACCGCCGTGTCCCGGCCCGGCCGCGGAGA
GCCCCGCTTCATCGCAGTGGGCTACGTGGACGACACGCAGTTCGTGCGGTTCGACAGCGACGCC
GCGAGTCCAAGAGGGGAGCCGCGGGCGCCGTGGGTGGAGCAGGAGGGGCCGGAGTATTGGGACC
GGGAGACACAGAAGTACAAGCGCCAGGCACAGGCTGACCGAGTGAGCCTGCGGAACCTGCGCGG
CTACTACAACCAGAGCGAGGCCGGGTCTCACACCCTCCAGAGGATGTACGGCTGCGACCTGGGG
CCCGACGGGCGCCTCCTCCGCGGGTATGACCAGTCCGCCTACGACGGCAAGGATTACATCGCCC
TGAACGAGGACCTGCGCTCCTGGACCGCTGCGGACACGGCGGCTCAGATCACCCAGCGCAAGTG
GGAGGCGGCCCGTGAGGCGGAGCAGTGGAGAGCCTACCTGGAGGGCACGTGCGTGGAGTGGCTC
CGCAGATACCTGGAGAACGGGAAGGAGACGCTGCAGCGCGCGGAACACCCAAAGACACACGTGA
CCCACCATCCCGTCTCTGACCATGAGGCCACCCTGAGGTGCTGGGCCCTGGGCTTCTACCCTGC
GGAGATCACACTGACCTGGCAGCGGGATGGCGAGGACCAAACTCAGGACACCGAGCTTGTGGAG
ACCAGGCCAGCAGGAGATGGAACCTTCCAGAAGTGGGCAGCTGTGGTGGTGCCTTCTGGAGAAG
AGCAGAGATACACGTGCCATGTGCAGCACGAGGGGCTGCCAGAGCCCCTCACCCTGAGATGGGA
GCCATCTTCCCAGCCCACCATCCCCATCATGGGCATCGTTGCTGGCCTGGCTGTCCTGGCTGTC
CTAGCTGTCCTAGGAGCTGTGATGGCTGTTGTGATGTGTAGGAGGAAGAGCTCAGGTGGAAAAG
GAGGGAGCTGCTCTCAGGCTGCGTCCAGCAACAGTGCCCAGGGCTCTGATGAGTCTCTCATCGC
TTGTAAAGCCTGA.

By “MHC class I polypeptide-related sequence A (MICA) polypeptide” is meant a protein having at least about 60%, 70%, or 85% amino acid sequence identity to GenBank Accession No. AAA21718.1, which is provided below, or a fragment thereof having NKG2D receptor binding activity.

>AAA21718.1 MHC class I-related protein
[Homo sapiens]
(SEQ ID NO: 1030)
MGLGPVFLLLAGIFPFAPPGAAAEPHSLRYNLTVLSWDGSVQSGFLTEVH
LDGQPFLRCDRQKCRAKPQGQWAEDVLGNKTWDRETRDLTGNGKDLRMTL
AHIKDQKEGLHSLQEIRVCEIHEDNSTRSSQHFYYDGELFLSQNLETKEW
TMPQSSRAQTLAMNVRNFLKEDAMKTKTHYHAMHADCLQELRRYLKSGVV
LRRTVPPMVNVTRSEASEGNITVTCRASGFYPWNITLSWRQDGVSLSHDT
QQWGDVLPDGNGTYQTWVATRICQGEEQRFTCYMEHSGNHSTHPVPSGKV
LVLQSHWQTFHVSAVAAAAIFVIIIFYVRCCKKKTSAAEGPELVSLQVLD
QHPVGTSDHRDATQLGFQPLMSDLGSTGSTEGA

By “MHC class I polypeptide-related sequence A (MICA) polynucleotide” is meant a nucleic acid molecule encoding an MICA polypeptide, as well as the introns, exons, 3′ untranslated regions, 5′ untranslated regions, and regulatory sequences associated with its expression, or fragments thereof. An exemplary MICA polynucleotide sequence is provided at Genbank Accession No. L14848.1, which is provided below.

>L14848.1:40-1191 Human MHC class I-related protein mRNA, complete cds
(SEQ ID NO: 1031)
ATGGGGCTGGGCCCGGTCTTCCTGCTTCTGGCTGGCATCTTCCCTTTTGCACCTCCGGGAGCTG
CTGCTGAGCCCCACAGTCTTCGTTATAACCTCACGGTGCTGTCCTGGGATGGATCTGTGCAGTC
AGGGTTTCTCACTGAGGTACATCTGGATGGTCAGCCCTTCCTGCGCTGTGACAGGCAGAAATGC
AGGGCAAAGCCCCAGGGACAGTGGGCAGAAGATGTCCTGGGAAATAAGACATGGGACAGAGAGA
CCAGAGACTTGACAGGGAACGGAAAGGACCTCAGGATGACCCTGGCTCATATCAAGGACCAGAA
AGAAGGCTTGCATTCCCTCCAGGAGATTAGGGTCTGTGAGATCCATGAAGACAACAGCACCAGG
AGCTCCCAGCATTTCTACTACGATGGGGAGCTCTTCCTCTCCCAAAACCTGGAGACTAAGGAAT
GGACAATGCCCCAGTCCTCCAGAGCTCAGACCTTGGCCATGAACGTCAGGAATTTCTTGAAGGA
AGATGCCATGAAGACCAAGACACACTATCACGCTATGCATGCAGACTGCCTGCAGGAACTACGG
CGATATCTAAAATCCGGCGTAGTCCTGAGGAGAACAGTGCCCCCCATGGTGAATGTCACCCGCA
GCGAGGCCTCAGAGGGCAACATTACCGTGACATGCAGGGCTTCTGGCTTCTATCCCTGGAATAT
CACACTGAGCTGGCGTCAGGATGGGGTATCTTTGAGCCACGACACCCAGCAGTGGGGGGATGTC
CTGCCTGATGGGAATGGAACCTACCAGACCTGGGTGGCCACCAGGATTTGCCAAGGAGAGGAGC
AGAGGTTCACCTGCTACATGGAACACAGCGGGAATCACAGCACTCACCCTGTGCCCTCTGGGAA
AGTGCTGGTGCTTCAGAGTCATTGGCAGACATTCCATGTTTCTGCTGTTGCTGCTGCTGCTATT
TTTGTTATTATTATTTTCTATGTCCGTTGTTGTAAGAAGAAAACATCAGCTGCAGAGGGTCCAG
AGCTCGTGAGCCTGCAGGTCCTGGATCAACACCCAGTTGGGACGAGTGACCACAGGGATGCCAC
ACAGCTCGGATTTCAGCCTCTGATGTCAGATCTTGGGTCCACTGGCTCCACTGAGGGCGCCTAG.
The MICA gene corresponds to Ensembl: ENSG00000204520.

By “MHC class I polypeptide-related sequence B (MICB) polypeptide” is meant a protein having at least about 85% amino acid sequence identity to NCBI Ref. Seq. Accession No. NP_005922.2, which is provided below, or a fragment thereof having NKG2D receptor binding activity.

(SEQ ID NO: 1032)
>NP_005922.2 MHC class I polypeptide-related
sequence B isoform 1 precursor [Homo sapiens]
MGLGRVLLFLAVAFPFAPPAAAAEPHSLRYNLMVLSQDGSVQSGFLAEG
HLDGQPFLRYDRQKRRAKPQGQWAENVLGAKTWDTETEDLTENGQDLRR
TLTHIKDQKGGLHSLQEIRVCEIHEDSSTRGSRHFYYDGELFLSQNLET
QESTVPQSSRAQTLAMNVTNFWKEDAMKTKTHYRAMQADCLQKLQRYLK
SGVAIRRTVPPMVNVTCSEVSEGNITVTCRASSFYPRNITLTWRQDGVS
LSHNTQQWGDVLPDGNGTYQTWVATRIRQGEEQRFTCYMEHSGNHGTHP
VPSGKALVLQSQRTDFPYVSAAMPCFVIIIILCVPCCKKKTSAAEGPEL
VSLQVLDQHPVGTGDHRDAAQLGFQPLMSATGSTGSTEGT

By “MHC class I polypeptide-related sequence B (MICB) polynucleotide” is meant a nucleic acid molecule encoding an MICB polypeptide, as well as the introns, exons, 3′ untranslated regions, 5′ untranslated regions, and regulatory sequences associated with its expression, or fragments thereof. An exemplary MICB polynucleotide sequence is provided at NCBI Ref. Seq. Accession No. NM_005931.5, which is provided below.

>NM _005931.5:50-1201 Homo sapiens MHC class I polypeptide-related
sequence B (MICB), transcript variant 1, mRNA
(SEQ ID NO: 1033)
ATGGGGCTGGGCCGGGTCCTGCTGTTTCTGGCCGTCGCCTTCCCTTTTGCACCCCCGGCAGCCG
CCGCTGAGCCCCACAGTCTTCGTTACAACCTCATGGTGCTGTCCCAGGATGGATCTGTGCAGTC
AGGGTTTCTCGCTGAGGGACATCTGGATGGTCAGCCCTTCCTGCGCTATGACAGGCAGAAACGC
AGGGCAAAGCCCCAGGGACAGTGGGCAGAAAATGTCCTGGGAGCTAAGACCTGGGACACAGAGA
CCGAGGACTTGACAGAGAATGGGCAAGACCTCAGGAGGACCCTGACTCATATCAAGGACCAGAA
AGGAGGCTTGCATTCCCTCCAGGAGATTAGGGTCTGTGAGATCCATGAAGACAGCAGCACCAGG
GGCTCCCGGCATTTCTACTACGATGGGGAGCTCTTCCTCTCCCAAAACCTGGAGACTCAAGAAT
CGACAGTGCCCCAGTCCTCCAGAGCTCAGACCTTGGCTATGAACGTCACAAATTTCTGGAAGGA
AGATGCCATGAAGACCAAGACACACTATCGCGCTATGCAGGCAGACTGCCTGCAGAAACTACAG
CGATATCTGAAATCCGGGGTGGCCATCAGGAGAACAGTGCCCCCCATGGTGAATGTCACCTGCA
GCGAGGTCTCAGAGGGCAACATCACCGTGACATGCAGGGCTTCCAGCTTCTATCCCCGGAATAT
CACACTGACCTGGCGTCAGGATGGGGTATCTTTGAGCCACAACACCCAGCAGTGGGGGGATGTC
CTGCCTGATGGGAATGGAACCTACCAGACCTGGGTGGCCACCAGGATTCGCCAAGGAGAGGAGC
AGAGGTTCACCTGCTACATGGAACACAGCGGGAATCACGGCACTCACCCTGTGCCCTCTGGGAA
GGCGCTGGTGCTTCAGAGTCAACGGACAGACTTTCCATATGTTTCTGCTGCTATGCCATGTTTT
GTTATTATTATTATTCTCTGTGTCCCTTGTTGCAAGAAGAAAACATCAGCGGCAGAGGGTCCAG
AGCTTGTGAGCCTGCAGGTCCTGGATCAACACCCAGTTGGGACAGGAGACCACAGGGATGCAGC
ACAGCTGGGATTTCAGCCTCTGATGTCAGCTACTGGGTCCACTGGTTCCACTGAGGGCACCTAG.
The MICB gene corresponds to Ensembl: ENSG00000204516.

By “nectin cell adhesion molecule 2 (Nectin-2) polypeptide” is meant a protein having at least about 85% amino acid sequence identity to NCBI Ref. Seq. Accession No. NP_001036189.1, which is provided below, or a fragment thereof having immunomodulatory and/or cell adhesive activity.

>NP_001036189.1 nectin-2 isoform delta precursor
[Homo sapiens]
(SEQ ID NO: 1034)
MARAAALLPSRSPPTPLLWPLLLLLLLETGAQDVRVQVLPEVRGQLGGTV
ELPCHLLPPVPGLYISLVTWQRPDAPANHQNVAAFHPKMGPSFPSPKPGS
ERLSFVSAKQSTGQDTEAELQDATLALHGLTVEDEGNYTCEFATFPKGSV
RGMTWLRVIAKPKNQAEAQKVTFSQDPTTVALCISKEGRPPARISWLSSL
DWEAKETQVSGTLAGTVTVTSRFTLVPSGRADGVTVTCKVEHESFEEPAL
IPVTLSVRYPPEVSISGYDDNWYLGRTDATLSCDVRSNPEPTGYDWSTTS
GTFPTSAVAQGSQLVIHAVDSLFNTTFVCTVTNAVGMGRAEQVIFVRETP
NTAGAGATGGIIGGIIAAIIATAVAATGILICRQQRKEQTLQGAEEDEDL
EGPPSYKPPTPKAKLEAQEMPSQLFTLGASEHSPLKTPYFDAGASCTEQE
MPRYHELPTLEERSGPLHPGATSLGSPIPVPPGPPAVEDVSLDLEDEEGE
EEEEYLDKINPIYDALSYSSPSDSYQGKGFVMSRAMYV

By “nectin cell adhesion molecule 2 (Nectin-2) polynucleotide” is meant a nucleic acid molecule encoding an Nectin-2 polypeptide, as well as the introns, exons, 3′ untranslated regions, 5′ untranslated regions, and regulatory sequences associated with its expression, or fragments thereof. An exemplary Nectin-2 polynucleotide sequence is provided at NCBI Ref. Seq. Accession No. NP_001036189.1, which is provided below.

>NM_001042724.2:230-1846 Homo sapiens nectin cell adhesion
molecule 2 (NECTIN2), transcript variant delta, mRNA
(SEQ ID NO: 1035)
ATGGCCCGGGCCGCTGCCCTCCTGCCGTCGAGATCGCCGCCGACGCCGCTGCTGTGGCCGCTGC
TGCTGCTGCTGCTCCTGGAAACCGGAGCCCAGGATGTGCGAGTTCAAGTGCTACCCGAGGTGCG
AGGCCAGCTCGGGGGCACCGTGGAGCTGCCGTGCCACCTGCTGCCACCTGTTCCTGGACTGTAC
ATCTCCCTGGTGACCTGGCAGCGCCCAGATGCACCTGCGAACCACCAGAATGTGGCCGCCTTCC
ACCCTAAGATGGGTCCCAGCTTCCCCAGCCCGAAGCCTGGCAGCGAGCGGCTGTCCTTCGTCTC
TGCCAAGCAGAGCACTGGGCAAGACACAGAGGCAGAGCTCCAGGACGCCACGCTGGCCCTCCAC
GGGCTCACGGTGGAGGACGAGGGCAACTACACTTGCGAGTTTGCCACCTTCCCCAAGGGGTCCG
TCCGAGGGATGACCTGGCTCAGAGTCATAGCCAAGCCCAAGAACCAAGCTGAGGCCCAGAAGGT
CACGTTCAGCCAGGACCCTACGACAGTGGCCCTCTGCATCTCCAAAGAGGGCCGCCCACCTGCC
CGGATCTCCTGGCTCTCATCCCTGGACTGGGAAGCCAAAGAGACTCAGGTGTCAGGGACCCTGG
CCGGAACTGTCACTGTCACCAGCCGCTTCACCTTGGTGCCCTCGGGCCGAGCAGATGGTGTCAC
GGTCACCTGCAAAGTGGAGCATGAGAGCTTCGAGGAACCAGCCCTGATACCTGTGACCCTCTCT
GTACGCTACCCTCCTGAAGTGTCCATCTCCGGCTATGATGACAACTGGTACCTCGGCCGTACTG
ATGCCACCCTGAGCTGTGACGTCCGCAGCAACCCAGAGCCCACGGGCTATGACTGGAGCACGAC
CTCAGGCACCTTCCCGACCTCCGCAGTGGCCCAGGGCTCCCAGCTGGTCATCCACGCAGTGGAC
AGTCTGTTCAATACCACCTTCGTCTGCACAGTCACCAATGCCGTGGGCATGGGCCGCGCTGAGC
AGGTCATCTTTGTCCGAGAGACCCCCAACACAGCAGGCGCAGGGGCCACAGGCGGCATCATCGG
GGGCATCATCGCCGCCATCATTGCTACTGCTGTGGCTGCCACGGGCATCCTTATCTGCCGGCAG
CAGCGGAAGGAGCAGACGCTGCAGGGGGCAGAGGAGGACGAAGACCTGGAGGGACCTCCCTCCT
ACAAGCCACCGACCCCAAAAGCGAAGCTGGAGGCACAGGAGATGCCCTCCCAGCTCTTCACTCT
GGGGGCCTCGGAGCACAGCCCACTCAAGACCCCCTACTTTGATGCTGGCGCCTCATGCACTGAG
CAGGAAATGCCTCGATACCATGAGCTGCCCACCTTGGAAGAACGGTCAGGACCCTTGCACCCTG
GAGCCACAAGCCTGGGGTCCCCCATCCCGGTGCCTCCAGGGCCACCTGCTGTGGAAGACGTTTC
CCTGGATCTAGAGGATGAGGAGGGGGAGGAGGAGGAAGAGTATCTGGACAAGATCAACCCCATC
TATGATGCTCTGTCCTATAGCAGCCCCTCTGATTCCTACCAGGGCAAAGGCTTTGTCATGTCCC
GGGCCATGTATGTGTGA
The Nectin-2 gene corresponds to ENSG00000130202.10.

By “NOD-like receptor (NLR) family, caspase recruitment (CARD) domain containing 5 (class-I transcriptional activator) (NLRC5 (CITA)) polypeptide” is meant a protein having at least about 85% amino acid sequence identity to NCBI Ref. Seq. Accession No. NP_115582.4, which is provided below, or a fragment thereof having immunomodulatory activity.

>NP_115582.4 protein NLRC5 isoform 1 [Homo sapiens]
(SEQ ID NO: 1036)
MDPVGLQLGNKNLWSCLVRLLTKDPEWLNAKMKFFLPNTDLDSRNETLDPEQRVILQLNKLHVQ
GSDTWQSFIHCVCMQLEVPLDLEVLLLSTFGYDDGFTSQLGAEGKSQPESQLHHGLKRPHQSCG
SSPRRKQCKKQQLELAKKYLQLLRTSAQQRYRSQIPGSGQPHAFHQVYVPPILRRATASLDTPE
GAIMGDVKVEDGADVSISDLFNTRVNKGPRVTVLLGKAGMGKTTLAHRLCQKWAEGHLNCFQAL
FLFEFRQLNLITRFLTPSELLFDLYLSPESDHDTVFQYLEKNADQVLLIFDGLDEALQPMGPDG
PGPVLTLFSHLCNGTLLPGCRVMATSRPGKLPACLPAEAAMVHMLGFDGPRVEEYVNHFFSAQP
SREGALVELQTNGRLRSLCAVPALCQVACLCLHHLLPDHAPGQSVALLPNMTQLYMQMVLALSP
PGHLPTSSLLDLGEVALRGLETGKVIFYAKDIAPPLIAFGATHSLLTSFCVCTGPGHQQTGYAF
THLSLQEFLAALHLMASPKVNKDTLTQYVTLHSRWVQRTKARLGLSDHLPTFLAGLASCTCRPF
LSHLAQGNEDCVGAKQAAVVQVLKKLATRKLTGPKVVELCHCVDETQEPELASLTAQSLPYQLP
FHNFPLTCTDLATLTNILEHREAPIHLDFDGCPLEPHCPEALVGCGQIENLSFKSRKCGDAFAE
ALSRSLPTMGRLQMLGLAGSKITARGISHLVKALPLCPQLKEVSFRDNQLSDQVVLNIVEVLPH
LPRLRKLDLSSNSICVSTLLCLARVAVTCPTVRMLQAREADLIFLLSPPTETTAELQRAPDLQE
SDGQRKGAQSRSLTLRLQKCQLQVHDAEALIALLQEGPHLEEVDLSGNQLEDEGCRLMAEAASQ
LHIARKLDLSNNGLSVAGVHCVLRAVSACWTLAELHISLQHKTVIFMFAQEPEEQKGPQERAAF
LDSLMLQMPSELPLSSRRMRLTHCGLQEKHLEQLCKALGGSCHLGHLHLDFSGNALGDEGAARL
AQLLPGLGALQSLNLSENGLSLDAVLGLVRCFSTLQWLFRLDISFESQHILLRGDKTSRDMWAT
GSLPDFPAAAKFLGFRQRCIPRSLCLSECPLEPPSLTRLCATLKDCPGPLELQLSCEFLSDQSL
ETLLDCLPQLPQLSLLQLSQTGLSPKSPFLLANTLSLCPRVKKVDLRSLHHATLHFRSNEEEEG
VCCGRFTGCSLSQEHVESLCWLLSKCKDLSQVDLSANLLGDSGLRCLLECLPQVPISGLLDLSH
NSISQESALYLLETLPSCPRVREASVNLGSEQSFRIHFSREDQAGKTLRLSECSFRPEHVSRLA
TGLSKSLQLTELTLTQCCLGQKQLAILLSLVGRPAGLFSLRVQEPWADRARVLSLLEVCAQASG
SVTEISISETQQQLCVQLEFPRQEENPEAVALRLAHCDLGAHHSLLVGQLMETCARLQQLSLSQ
VNLCEDDDASSLLLQSLLLSLSELKTFRLTSSCVSTEGLAHLASGLGHCHHLEELDLSNNQFDE
EGTKALMRALEGKWMLKRLDLSHLLLNSSTLALLTHRLSQMTCLQSLRLNRNSIGDVGCCHLSE
ALRAATSLEELDLSHNQIGDAGVQHLATILPGLPELRKIDLSGNSISSAGGVQLAESLVLCRRL
EELMLGCNALGDPTALGLAQELPQHLRVLHLPFSHLGPGGALSLAQALDGSPHLEEISLAENNL
AGGVLRFCMELPLLRQIDLVSCKIDNQTAKLLTSSFTSCPALEVILLSWNLLGDEAAAELAQVL
PQMGRLKRVDLEKNQITALGAWLLAEGLAQGSSIQVIRLWNNPIPCDMAQHLKSQEPRLDFAFF
DNQPQAPWGT.

By “NLR family CARD domain containing 5 (class-I transcriptional activator) (NLRC5 (CITA)) polynucleotide” is meant a nucleic acid molecule encoding an NLRC5 polypeptide, as well as the introns, exons, 3′ untranslated regions, 5′ untranslated regions, and regulatory sequences associated with its expression, or fragments thereof. An exemplary NLRC5 polynucleotide sequence is provided at NCBI Ref. Seq. Accession No. NM_032206.5, which is provided below.

>NM_032206.5:167-5767 Homo sapiens NLR family CARD domain
containing 5 (NLRC5), transcript variant 1, mRNA
(SEQ ID NO: 1037)
ATGGACCCCGTTGGCCTCCAGCTCGGCAACAAGAACCTGTGGAGCTGTCTTGTGAGGCTGCTCA
CCAAAGACCCAGAATGGCTGAACGCCAAGATGAAGTTCTTCCTCCCCAACACGGACCTGGATTC
CAGGAACGAGACCTTGGACCCTGAACAGAGAGTCATCCTGCAACTCAACAAGCTGCATGTCCAG
GGTTCGGACACCTGGCAGTCTTTCATTCATTGTGTGTGCATGCAGCTGGAGGTGCCTCTGGACC
TGGAGGTGCTGCTGCTGAGTACTTTTGGCTATGATGATGGGTTCACCAGCCAGCTGGGAGCTGA
GGGGAAAAGCCAACCTGAATCTCAGCTCCACCATGGCCTGAAGCGCCCACATCAGAGCTGTGGG
TCCTCACCCCGCCGGAAGCAGTGCAAGAAGCAGCAGCTAGAGTTGGCCAAGAAGTACCTGCAGC
TCCTGCGGACCTCTGCCCAGCAGCGCTACAGGAGCCAAATCCCTGGGTCAGGGCAGCCCCACGC
CTTCCACCAGGTCTATGTCCCTCCAATCCTGCGCCGGGCCACAGCATCCTTAGACACTCCGGAG
GGGGCCATTATGGGGGACGTCAAGGTGGAAGATGGTGCTGACGTGAGCATCTCGGACCTCTTCA
ACACCAGGGTTAACAAGGGCCCGAGGGTGACCGTGCTTTTGGGGAAGGCTGGCATGGGCAAGAC
CACGCTGGCCCACCGGCTCTGCCAGAAGTGGGCAGAGGGCCATCTGAACTGTTTCCAGGCCCTG
TTCCTTTTTGAATTCCGCCAGCTCAACTTGATCACGAGGTTCCTGACACCGTCCGAGCTCCTTT
TTGATCTGTACCTGAGCCCTGAATCGGACCACGACACTGTCTTCCAGTACCTGGAGAAGAACGC
TGACCAAGTCCTGCTGATCTTTGATGGGCTAGATGAGGCCCTCCAGCCTATGGGTCCTGATGGC
CCAGGCCCAGTCCTCACCCTTTTCTCCCATCTCTGCAATGGGACCCTCCTGCCTGGCTGCCGGG
TGATGGCTACCTCCCGTCCAGGGAAGCTGCCTGCCTGCCTGCCTGCAGAGGCAGCCATGGTCCA
CATGTTGGGCTTTGATGGGCCACGGGTGGAAGAATATGTGAATCACTTCTTCAGCGCCCAGCCA
TCGCGGGAGGGGGCCCTGGTGGAGTTACAGACAAATGGACGTCTCCGAAGCCTGTGTGCGGTGC
CCGCACTGTGCCAAGTCGCCTGTCTCTGCCTCCACCATCTGCTTCCTGACCACGCCCCAGGCCA
GTCTGTGGCCCTCCTGCCCAACATGACTCAGCTCTATATGCAGATGGTGCTCGCCCTCAGCCCC
CCTGGGCACTTGCCCACCTCGTCCCTACTGGACCTGGGGGAGGTGGCCCTGAGGGGCCTGGAGA
CAGGGAAGGTTATCTTCTATGCAAAAGATATTGCTCCACCCTTGATAGCTTTTGGGGCCACTCA
CAGCCTGCTGACTTCCTTCTGCGTCTGCACAGGCCCTGGGCACCAGCAGACAGGCTATGCTTTC
ACCCACCTCAGCCTGCAGGAGTTTCTTGCTGCCCTGCACCTGATGGCCAGCCCCAAGGTGAACA
AAGACACACTTACCCAGTATGTTACCCTCCATTCCCGCTGGGTACAGCGGACCAAAGCTAGACT
GGGCCTCTCAGACCACCTCCCCACCTTCCTGGCGGGCCTGGCATCCTGCACCTGCCGCCCCTTC
CTTAGCCACCTGGCGCAGGGCAATGAGGACTGTGTGGGTGCCAAGCAGGCTGCTGTAGTGCAGG
TGTTGAAGAAGTTGGCCACCCGCAAGCTCACAGGGCCAAAGGTTGTAGAGCTGTGTCACTGTGT
GGATGAGACACAGGAGCCTGAGCTGGCCAGTCTCACCGCACAAAGCCTCCCCTATCAACTGCCC
TTCCACAATTTCCCACTGACCTGCACCGACCTGGCCACCCTGACCAACATCCTAGAGCACAGGG
AGGCCCCCATCCACCTGGATTTTGATGGCTGTCCCCTGGAGCCCCACTGCCCTGAGGCTCTGGT
AGGCTGTGGGCAGATAGAGAATCTCAGCTTTAAGAGCAGGAAGTGTGGGGATGCCTTTGCAGAA
GCCCTCTCCAGGAGCTTGCCGACAATGGGGAGGCTGCAGATGCTGGGGTTAGCAGGAAGTAAAA
TCACTGCCCGAGGCATCAGCCACCTGGTGAAAGCTTTGCCTCTCTGTCCACAGCTGAAAGAAGT
CAGTTTTCGGGACAACCAGCTCAGTGACCAGGTGGTGCTGAACATTGTGGAGGTTCTCCCTCAC
CTACCACGGCTCCGGAAGCTTGACCTGAGCAGCAACAGCATCTGCGTGTCAACCCTACTCTGCT
TGGCAAGGGTGGCAGTCACGTGTCCTACCGTCAGGATGCTTCAGGCCAGGGAGGCGGACCTCAT
CTTCCTTCTTTCCCCGCCCACAGAGACAACTGCAGAGCTACAAAGAGCTCCAGACCTGCAGGAA
AGTGACGGCCAGAGGAAAGGGGCTCAGAGCAGAAGCTTGACGCTCAGGCTGCAGAAGTGTCAGC
TCCAGGTCCACGATGCGGAGGCCCTCATAGCCCTGCTCCAGGAAGGCCCTCACCTGGAGGAAGT
GGACCTCTCAGGGAACCAGCTGGAAGATGAAGGCTGTCGGCTGATGGCAGAGGCTGCATCCCAG
CTGCACATCGCCAGGAAGCTGGACCTCAGTAACAACGGGCTTTCTGTGGCCGGGGTGCATTGTG
TGCTGAGGGCCGTGAGTGCGTGCTGGACCCTGGCAGAGCTGCACATCAGCCTGCAGCACAAAAC
TGTGATCTTCATGTTTGCCCAGGAGCCAGAGGAGCAGAAGGGGCCCCAGGAGAGGGCTGCATTT
CTTGACAGCCTCATGCTCCAGATGCCCTCTGAGCTGCCTCTGAGCTCCCGAAGGATGAGGCTGA
CACATTGTGGCCTCCAAGAAAAGCACCTAGAGCAGCTCTGCAAGGCTCTGGGAGGAAGCTGCCA
CCTCGGTCACCTCCACCTCGACTTCTCAGGCAATGCTCTGGGGGATGAAGGTGCAGCCCGGCTG
GCTCAGCTGCTCCCAGGGCTGGGAGCTCTGCAGTCCTTGAACCTCAGTGAGAACGGTTTGTCCC
TGGATGCCGTGTTGGGTTTGGTTCGGTGCTTCTCCACTCTGCAGTGGCTCTTCCGCTTGGACAT
CAGCTTTGAAAGCCAACACATCCTCCTGAGAGGGGACAAGACAAGCAGGGATATGTGGGCCACT
GGATCTTTGCCAGACTTCCCAGCTGCAGCCAAGTTCTTAGGGTTCCGTCAGCGCTGCATCCCCA
GGAGCCTCTGCCTCAGTGAGTGTCCTCTGGAGCCCCCAAGCCTCACCCGCCTCTGTGCCACTCT
GAAGGACTGCCCGGGACCCCTGGAACTGCAATTGTCCTGTGAGTTCCTGAGTGACCAGAGCCTG
GAGACTCTACTGGACTGCTTACCTCAACTCCCTCAGCTGAGCCTGCTGCAGCTGAGCCAGACGG
GACTGTCCCCGAAAAGCCCCTTCCTGCTGGCCAACACCTTAAGCCTGTGTCCACGGGTTAAAAA
GGTGGATCTCAGGTCCCTGCACCATGCAACTTTGCACTTCAGATCCAACGAGGAGGAGGAAGGC
GTGTGCTGTGGCAGGTTCACAGGCTGCAGCCTCAGCCAGGAGCACGTAGAGTCACTCTGCTGGT
TGCTGAGCAAGTGTAAAGACCTCAGCCAGGTGGATCTCTCAGCAAACCTGCTGGGCGACAGCGG
ACTCAGATGCCTTCTGGAATGTCTGCCGCAGGTGCCCATCTCCGGTTTGCTTGATCTGAGTCAC
AACAGCATTTCTCAGGAAAGTGCCCTGTACCTGCTGGAGACACTGCCCTCCTGCCCACGTGTCC
GGGAGGCCTCAGTGAACCTGGGCTCTGAGCAGAGCTTCCGGATTCACTTCTCCAGAGAGGACCA
GGCTGGGAAGACACTCAGGCTAAGTGAGTGCAGCTTCCGGCCAGAGCACGTGTCCAGGCTGGCC
ACCGGCTTGAGCAAGTCCCTGCAGCTGACGGAGCTCACGCTGACCCAGTGCTGCCTGGGCCAGA
AGCAGCTGGCCATCCTCCTGAGCTTGGTGGGGCGACCCGCAGGGCTGTTCAGCCTCAGGGTGCA
GGAGCCGTGGGCGGACAGAGCCAGGGTTCTCTCCCTGTTAGAAGTCTGCGCCCAGGCCTCAGGC
AGTGTCACTGAAATCAGCATCTCCGAGACCCAGCAGCAGCTCTGTGTCCAGCTGGAATTTCCTC
GCCAGGAAGAGAATCCAGAAGCTGTGGCACTCAGGTTGGCTCACTGTGACCTTGGAGCCCACCA
CAGCCTTCTTGTCGGGCAGCTGATGGAGACATGTGCCAGGCTGCAGCAGCTCAGCTTGTCTCAG
GTTAACCTCTGTGAGGACGATGATGCCAGTTCCCTGCTGCTGCAGAGCCTCCTGCTGTCCCTCT
CTGAGCTGAAGACATTTCGGCTGACCTCCAGCTGTGTGAGCACCGAGGGCCTCGCCCACCTGGC
ATCTGGTCTGGGCCACTGCCACCACTTGGAGGAGCTGGACTTGTCTAACAATCAATTTGATGAG
GAGGGCACCAAGGCGCTGATGAGGGCCCTTGAGGGGAAATGGATGCTAAAGAGGCTGGACCTCA
GTCACCTTCTGCTGAACAGCTCCACCTTGGCCTTGCTTACTCACAGACTAAGCCAGATGACCTG
CCTGCAGAGCCTCAGACTGAACAGGAACAGTATCGGTGATGTCGGTTGCTGCCACCTTTCTGAG
GCTCTCAGGGCTGCCACCAGCCTAGAGGAGCTGGACTTGAGCCACAACCAGATTGGAGACGCTG
GTGTCCAGCACTTAGCTACCATCCTGCCTGGGCTGCCAGAGCTCAGGAAGATAGACCTCTCAGG
GAATAGCATCAGCTCAGCCGGGGGAGTGCAGTTGGCAGAGTCTCTCGTTCTTTGCAGGCGCCTG
GAGGAGTTGATGCTTGGCTGCAATGCCCTGGGGGATCCCACAGCCCTGGGGCTGGCTCAGGAGC
TGCCCCAGCACCTGAGGGTCCTACACCTACCATTCAGCCATCTGGGCCCAGGTGGGGCCCTGAG
CCTGGCCCAGGCCCTGGATGGATCCCCCCATTTGGAAGAGATCAGCTTGGCGGAAAACAACCTG
GCTGGAGGGGTCCTGCGTTTCTGTATGGAGCTCCCGCTGCTCAGACAGATAGACCTGGTTTCCT
GTAAGATTGACAACCAGACTGCCAAGCTCCTCACCTCCAGCTTCACGAGCTGCCCTGCCCTGGA
AGTAATCTTGCTGTCCTGGAATCTCCTCGGGGATGAGGCAGCTGCCGAGCTGGCCCAGGTGCTG
CCGCAGATGGGCCGGCTGAAGAGAGTGGACCTGGAGAAGAATCAGATCACAGCTTTGGGGGCCT
GGCTCCTGGCTGAAGGACTGGCCCAGGGGTCTAGCATCCAAGTCATCCGCCTCTGGAATAACCC
CATTCCCTGCGACATGGCCCAGCACCTGAAGAGCCAGGAGCCCAGGCTGGACTTTGCCTTCTTT
GACAACCAGCCCCAGGCCCCTTGGGGTACTTGA
The NLRC genecorresponds to Ensembl:ENSG00000140853 MIM:613537.

By “protein disulfide isomerase family A member 3 (PDIA3; ERp57) polypeptide” (previously known as phospholipase C-alpha) is meant a protein having at least about 85% amino acid sequence identity to GenBank Accession No. CAA89996.1, which is provided below, or a fragment thereof having immunomodulatory activity.

> CAA89996.1 protein disulfide isomerase
(alternatively BAA03759.1 phospholipase C-alpha) [Homo sapiens]
(SEQ ID NO: 1038)
MRLRRLALFPGVALLLAAARLAAASDVLELTDDNFESRISDTGSAGLMLVEFFAPWCGHCKRLA
PEYEAAATRLKGIVPLAKVDCTANTNTCNKYGVSGYPTLKIFRDGEEAGAYDGPRTADGIVSHL
KKQAGPASVPLRTEEEFKKFISDKDASIVGFFDDSFSEAHSEFLKAASNLRDNYRFAHTNVESL
VNEYDDNGEGIILFRPSHLTNKFEYKTVAYTEQKMTSGKIKKFIQENIFGICPHMTEDNKDLIQ
GKDLLIAYYDVDYEKDAKGSNYWRNRVMMVAKKFLDAGHKLNFAVASRKTFSHELSDFGLESTA
GEIPVVAIRTAKGEKFVMQEEFSRDGKALERFLQGYFGGNLKRYLKSDPIPESNDGPVKVVVAE
NFDEIVNNENKDVLIEFYAPWCGHCKNLEPKYKELGEKLSKDPNIVIAKMDATANDVPSPYEVR
GFPTIYFSPANKKLNPKKYEGGRELSDFISYLQREATNPPVIQEEKPKKKKKAQEDL.

By “protein disulfide isomerase family A member 3 (PDIA3; ERp57) polynucleotide” is meant a nucleic acid molecule encoding an PDIA3 polypeptide, as well as the introns, exons, 3′ untranslated regions, 5′ untranslated regions, and regulatory sequences associated with its expression, or fragments thereof. An exemplary PDIA3 polynucleotide sequence is provided at GenBank Accession No. D16234.1, which is provided below.

>D16234.1:68-1585 Homo sapiens mRNA for phospholipase C-alpha,
complete cds
(SEQ ID NO: 1039)
ATGCGCCTCCGCCGCCTAGCGCTGTTCCCGGGTGTGGCGCTGCTTCTTGCCGCGGCCCGCCTCG
CCGCTGCCTCCGACGTGCTAGAACTCACGGACGACAACTTCGAGAGTCGCATCTCCGACACGGG
CTCTGCGGGCCTCATGCTCGTCGAGTTCTTCGCTCCCTGGTGTGGACACTGCAAGAGACTTGCA
CCTGAGTATGAAGCTGCAGCTACCAGATTAAAAGGAATAGTCCCATTAGCAAAGGTTGATTGCA
CTGCCAACACTAACACCTGTAATAAATATGGAGTCAGTGGATATCCAACCCTGAAGATATTTAG
AGATGGTGAAGAAGCAGGTGCTTATGATGGACCTAGGACTGCTGATGGAATTGTCAGCCACTTG
AAGAAGCAGGCAGGACCAGCTTCAGTGCCTCTCAGGACTGAGGAAGAATTTAAGAAATTCATTA
GTGATAAAGATGCCTCTATAGTAGGTTTTTTCGATGATTCATTCAGTGAGGCTCACTCCGAGTT
CCTAAAAGCAGCCAGCAACTTGAGGGATAACTACCGATTTGCACATACGAATGTTGAGTCTCTG
GTGAACGAGTATGATGATAATGGAGAGGGTATCATCTTATTTCGTCCTTCACATCTCACTAACA
AGTTTGAGTACAAGACTGTGGCATATACAGAGCAAAAAATGACCAGTGGCAAAATTAAAAAGTT
TATCCAGGAAAACATTTTTGGTATCTGCCCTCACATGACAGAGGACAATAAAGATTTGATACAG
GGCAAGGACTTACTTATTGCTTACTATGATGTGGACTATGAAAAGGACGCTAAAGGTTCCAACT
ACTGGAGAAACAGGGTAATGATGGTGGCAAAGAAATTCCTGGATGCTGGGCACAAACTCAACTT
TGCTGTAGCTAGCCGCAAAACCTTTAGCCATGAACTTTCTGATTTTGGCTTGGAGAGCACTGCT
GGAGAGATTCCTGTTGTTGCTATCAGGACTGCTAAAGGAGAGAAGTTTGTCATGCAGGAGGAGT
TCTCGCGTGATGGGAAGGCTCTGGAGAGGTTCCTGCAGGGTTACTTTGGTGGCAATCTGAAGAG
ATACCTGAAGTCTGACCCTATCCCAGAGAGCAATGATGGGCCTGTGAAGGTAGTGGTAGCAGAG
AATTTTGATGAAATAGTGAATAATGAAAATAAAGATGTGCTGATTGAATTTTATGCCCCTTGGT
GTGGTCATTGTAAGAACCTGGAGCCCAAGTATAAAGAACTTGGCGAGAAGCTCAGCAAAGACCC
AAATATCGTCATAGCCAAGATGGATGCCACAGCCAATGATGTGCCTTCTCCATATGAAGTCAGA
GGTTTTCCTACCATATACTTCTCTCCAGCCAACAAGAAGCTAAATCCAAAGAAATATGAAGGTG
GCCGTGAATTAAGTGATTTTATTAGCTATCTACAAAGAGAAGCTACAAACCCCCCTGTAATTCA
AGAAGAAAAACCCAAGAAGAAGAAGAAGGCACAGGAGGATCTCTAA.
The PDIA3 gene corresponds to Ensembl:ENSG00000167004.

By “UL16 binding protein 1-6 (ULBP) polypeptide” is meant a protein having at least about 85% amino acid sequence identity to GenBank Accession No. AAK13081.1, AAK13082.1, or AAK13083.1, AVP72463.1, or NCBI Ref. Seq. No. NP_001001788.2 or NP_570970.2, which are provided below, or a fragment thereof having NKG2D receptor binding activity.

>AAK13083.1 ULBP3 protein [Homo sapiens]
(SEQ ID NO: 1040)
MAAAASPAILPRLAILPYLLFDWSGTGRADAHSLWYNFTIIHLPRHGQQWCEVQSQVDQKNFLS
YDCGSDKVLSMGHLEEQLYATDAWGKQLEMLREVGQRLRLELADTELEDFTPSGPLTLQVRMSC
ECEADGYIRGSWQFSFDGRKFLLFDSNNRKWTVVHAGARRMKEKWEKDSGLTTFFKMVSMRDCK
SWLRDFLMHRKKRLEPTAPPTMAPGLAQPKAIATTLSPWSFLIILCFILPGI.
>AAK13082.1 ULBP2 protein [Homo sapiens]
(SEQ ID NO: 1041)
MAAAAATKILLCLPLLLLLSGWSRAGRADPHSLCYDITVIPKFRPGPRWCAVQGQVDEKTFLHY
DCGNKTVTPVSPLGKKLNVTTAWKAQNPVLREVVDILTEQLRDIQLENYTPKEPLTLQARMSCE
QKAEGHSSGSWQFSFDGQIFLLFDSEKRMWTTVHPGARKMKEKWENDKVVAMSFHYFSMGDCIG
WLEDFLMGMDSTLEPSAGAPLAMSSGTTQLRATATTLILCCLLIILPCFILPGI.
>AAK13081.1 ULBP1 protein [Homo sapiens]
(SEQ ID NO: 1042)
MAAAASPAFLLCLPLLHLLSGWSRAGWVDTHCLCYDFIITPKSRPEPQWCEVQGLVDERPFLHY
DCVNHKAKAFASLGKKVNVTKTWEEQTETLRDVVDFLKGQLLDIQVENLIPIEPLTLQARMSCE
HEAHGHGRGSWQFLFNGQKFLLFDSNNRKWTALHPGAKKMTEKWEKNRDVTMFFQKISLGDCKM
WLEEFLMYWEQMLDPTKPPSLAPGTTQPKAMATTLSPWSLLIIFLCFILAGR.
>AVP72463.1 ULBP4 [Homo sapiens]
(SEQ ID NO: 1080)
MRRISLTSSPVCLLLFLLLLLIALEIMVGGHSLCFNFTIKSLSRPGQPWCEAQVFLNKNLFLQY
NSDNNMVKPLGLLGKKVNATSTWGELTQTLGEVGRDLRMLLCDIKPQIKTSDPSTLQVEMFCQR
EAERCTGASWQFATNGEKSLLFDAMNMTWTVINHEASKIKETWKKDRGLEKYFRKLSKGDCDHW
LREFLGHWEAMPEPTVSPVNASDIHWSSSSLPDRWIILGAFILLVLMGIVLICVWWQNGRRST.
>NP 001001788.2 UL-16 binding protein 5 preproprotein [Homo sapiens]
(SEQ ID NO: 1081)
MAAAASPAFLLRLPLLLLLSSWCRTGLADPHSLCYDITVIPKFRPGPRWCAVQGQVDEKTFLHY
DCGSKTVTPVSPLGKKLNVTTAWKAQNPVLREVVDILTEQLLDIQLENYIPKEPLTLQARMSCE
QKAEGHGSGSWQLSFDGQIFLLFDSENRMWTTVHPGARKMKEKWENDKDMTMSFHYISMGDCTG
WLEDFLMGMDSTLEPSAGAPPTMSSGTAQPRATATTLILCCLLIMCLLICSRHSLTQSHGHHPQ
SLQPPPHPPLLHPTWLLRRVLWSDSYQIAKRPLSGGHVTRVTLPIIGDDSHSLPCPLALYTINN
GAARYSEPLQVSIS.
>NP 570970.2 UL16-binding protein 6 precursor [Homo sapiens]
(SEQ ID NO: 1082)
MAAAAIPALLLCLPLLFLLFGWSRARRDDPHSLCYDITVIPKFRPGPRWCAVQGQVDEKTFLHY
DCGNKTVTPVSPLGKKLNVTMAWKAQNPVLREVVDILTEQLLDIQLENYTPKEPLTLQARMSCE
QKAEGHSSGSWQFSIDGQTFLLFDSEKRMWTTVHPGARKMKEKWENDKDVAMSFHYISMGDCIG
WLEDFLMGMDSTLEPSAGAPLAMSSGTTQLRATATTLILCCLLIILPCFILPGI.

By “UL16 binding protein 1-6 (ULBP) polynucleotide” is meant a nucleic acid molecule encoding an ULBP polypeptide, as well as the introns, exons, 3′ untranslated regions, 5′ untranslated regions, and regulatory sequences associated with its expression, or fragments thereof. An exemplary ULBP polynucleotide sequence is provided at GenBank Accession Nos. AF304379.1, AF304378.1, AF304377.1, MH020173.1, and NCBI Ref. Seq. Nos. NM_001001788.4 and NM_130900.3 which are provided below.

>AF304379.1 Homo sapiens ULBP3 protein mRNA, complete cds
(SEQ ID NO: 1043)
ATGGCAGCGGCCGCCAGCCCCGCGATCCTTCCGCGCCTCGCGATTCTTCCGTACCTGCTATTCG
ACTGGTCCGGGACGGGGGGGGCCGACGCTCACTCTCTCTGGTATAACTTCACCATCATTCATTT
GCCCAGACATGGGCAACAGTGGTGTGAGGTCCAGAGCCAGGTGGATCAGAAGAATTTTCTCTCC
TATGACTGTGGCAGTGACAAGGTCTTATCTATGGGTCACCTAGAAGAGCAGCTGTATGCCACAG
ATGCCTGGGGAAAACAACTGGAAATGCTGAGAGAGGTGGGGCAGAGGCTCAGACTGGAACTGGC
TGACACTGAGCTGGAGGATTTCACACCCAGTGGACCCCTCACGCTGCAGGTCAGGATGTCTTGT
GAGTGTGAAGCCGATGGATACATCCGTGGATCTTGGCAGTTCAGCTTCGATGGACGGAAGTTCC
TCCTCTTTGACTCAAACAACAGAAAGTGGACAGTGGTTCACGCTGGAGCCAGGCGGATGAAAGA
GAAGTGGGAGAAGGATAGCGGACTGACCACCTTCTTCAAGATGGTCTCAATGAGAGACTGCAAG
AGCTGGCTTAGGGACTTCCTGATGCACAGGAAGAAGAGGCTGGAACCCACAGCACCACCCACCA
TGGCCCCAGGCTTAGCTCAACCCAAAGCCATAGCCACCACCCTCAGTCCCTGGAGCTTCCTCAT
CATCCTCTGCTTCATCCTCCCTGGCATCTGA.
>AF304378.1 Homo sapiens ULBP2 protein mRNA, complete cds
(SEQ ID NO: 1044)
ATGGCAGCAGCCGCCGCTACCAAGATCCTTCTGTGCCTCCCGCTTCTGCTCCTGCTGTCCGGCT
GGTCCCGGGCTGGGCGAGCCGACCCTCACTCTCTTTGCTATGACATCACCGTCATCCCTAAGTT
CAGACCTGGACCACGGTGGTGTGCGGTTCAAGGCCAGGTGGATGAAAAGACTTTTCTTCACTAT
GACTGTGGCAACAAGACAGTCACACCTGTCAGTCCCCTGGGGAAGAAACTAAATGTCACAACGG
CCTGGAAAGCACAGAACCCAGTACTGAGAGAGGTGGTGGACATACTTACAGAGCAACTGCGTGA
CATTCAGCTGGAGAATTACACACCCAAGGAACCCCTCACCCTGCAGGCAAGGATGTCTTGTGAG
CAGAAAGCTGAAGGACACAGCAGTGGATCTTGGCAGTTCAGTTTCGATGGGCAGATCTTCCTCC
TCTTTGACTCAGAGAAGAGAATGTGGACAACGGTTCATCCTGGAGCCAGAAAGATGAAAGAAAA
GTGGGAGAATGACAAGGTTGTGGCCATGTCCTTCCATTACTTCTCAATGGGAGACTGTATAGGA
TGGCTTGAGGACTTCTTGATGGGCATGGACAGCACCCTGGAGCCAAGTGCAGGAGCACCACTCG
CCATGTCCTCAGGCACAACCCAACTCAGGGCCACAGCCACCACCCTCATCCTTTGCTGCCTCCT
CATCATCCTCCCCTGCTTCATCCTCCCTGGCATCTGA.
>AF304377.1 Homo sapiens ULBP1 protein mRNA, complete cds
(SEQ ID NO: 1045)
ATGGCAGCGGCCGCCAGCCCCGCCTTCCTTCTGTGCCTCCCGCTTCTGCACCTGCTGTCTGGCT
GGTCCCGGGCAGGATGGGTCGACACACACTGTCTTTGCTATGACTTCATCATCACTCCTAAGTC
CAGACCTGAACCACAGTGGTGTGAAGTTCAAGGCCTGGTGGATGAAAGGCCTTTTCTTCACTAT
GACTGTGTTAACCACAAGGCCAAAGCCTTTGCTTCTCTGGGGAAGAAAGTCAATGTCACAAAAA
CCTGGGAAGAACAAACTGAAACACTAAGAGACGTGGTGGATTTCCTTAAAGGGCAACTGCTTGA
CATTCAAGTGGAGAATTTAATACCCATTGAGCCCCTCACCCTGCAGGCCAGGATGTCTTGTGAG
CATGAAGCCCATGGACACGGCAGAGGATCTTGGCAGTTCCTCTTCAATGGACAGAAGTTCCTCC
TCTTTGACTCAAACAACAGAAAGTGGACAGCACTTCATCCTGGAGCCAAGAAGATGACAGAGAA
GTGGGAGAAGAACAGGGATGTGACCATGTTCTTCCAGAAGATTTCACTGGGGGATTGTAAGATG
TGGCTTGAAGAATTTTTGATGTACTGGGAACAAATGCTGGATCCAACAAAACCACCCTCTCTGG
CCCCAGGCACAACCCAACCCAAGGCCATGGCCACCACCCTCAGTCCCTGGAGCCTTCTCATCAT
CTTCCTCTGCTTCATTCTAGCTGGCAGATGA.
>MH020173.1:64-831 Homo sapiens ULBP4 mRNA, complete cds
(SEQ ID NO: 1083)
ATGCGAAGAATATCCCTGACTTCTAGCCCTGTGTGCCTTCTTTTGTTTCTGCTGTTGCTACTAA
TAGCCTTGGAGATCATGGTTGGTGGTCACTCTCTTTGCTTCAACTTCACTATAAAATCATTGTC
CAGACCTGGACAGCCCTGGTGTGAAGCGCAGGTCTTCTTGAATAAAAATCTTTTCCTTCAGTAC
AACAGTGACAACAACATGGTCAAACCTCTGGGCCTCCTGGGGAAGAAGGTAAATGCCACCAGCA
CTTGGGGAGAATTGACCCAAACGCTGGGAGAAGTGGGGCGAGACCTCAGGATGCTCCTTTGTGA
CATCAAACCCCAGATAAAGACCAGTGATCCTTCCACTCTGCAAGTCGAGATGTTTTGTCAACGT
GAAGCAGAACGGTGCACTGGTGCATCCTGGCAGTTCGCCACCAATGGAGAGAAATCCCTCCTCT
TTGACGCAATGAACATGACCTGGACAGTAATTAATCATGAAGCCAGTAAGATCAAGGAGACATG
GAAGAAAGACAGAGGGCTGGAAAAGTATTTCAGGAAGCTCTCAAAGGGAGACTGCGATCACTGG
CTCAGGGAATTCTTAGGGCACTGGGAGGCAATGCCAGAACCGACAGTGTCACCAGTAAATGCTT
CAGATATCCACTGGTCTTCTTCTAGTCTACCAGATAGATGGATCATCCTGGGGGCATTCATCCT
GTTAGTTTTAATGGGAATTGTTCTCATCTGTGTCTGGTGGCAAAATGGCAGAAGATCCACCTAG.
>NM_001001788.4:112-1116 Homo sapiens retinoic acid early
transcript 1G (RAETIG), transcript variant 1, mRNA
(SEQ ID NO: 1084)
ATGGCAGCGGCCGCCAGCCCCGCGTTCCTTCTACGCCTCCCGCTTCTGCTCCTGCTGTCCAGCT
GGTGCAGGACCGGGCTGGCCGACCCTCACTCTCTTTGCTATGACATCACCGTCATCCCTAAGTT
CAGACCTGGACCACGGTGGTGTGCGGTTCAAGGCCAGGTGGATGAAAAGACTTTTCTTCACTAT
GACTGTGGCAGCAAGACAGTCACACCCGTCAGTCCCCTGGGGAAGAAACTAAATGTCACAACGG
CCTGGAAAGCACAGAACCCAGTACTGAGAGAGGTGGTGGACATACTTACAGAGCAACTGCTTGA
CATTCAGCTGGAGAATTACATACCCAAGGAACCCCTCACCCTGCAGGCCAGGATGTCTTGTGAG
CAGAAAGCCGAAGGACACGGCAGTGGATCTTGGCAGCTCAGTTTCGATGGACAGATCTTCCTCC
TCTTTGACTCAGAAAACAGAATGTGGACAACGGTTCATCCTGGAGCCAGAAAGATGAAAGAAAA
GTGGGAGAATGACAAGGATATGACCATGTCCTTCCATTACATCTCAATGGGAGACTGCACAGGA
TGGCTTGAGGACTTCTTGATGGGCATGGACAGCACCCTGGAGCCAAGTGCAGGAGCACCACCCA
CCATGTCCTCAGGCACAGCCCAACCCAGGGCCACGGCCACCACCCTCATCCTTTGCTGCCTCCT
CATCATGTGTCTCCTCATATGCTCCAGGCACAGTCTGACCCAAAGCCATGGCCACCACCCTCAG
TCCCTGCAGCCTCCTCCTCATCCTCCCCTGCTTCATCCTACCTGGCTGCTGAGGAGAGTCCTTT
GGAGTGACAGCTACCAAATAGCGAAGCGCCCCTTGTCTGGTGGACACGTGACTCGCGTGACTTT
ACCTATCATTGGAGACGACTCACACTCCTTACCCTGCCCTCTTGCCTTGTATACAATAAATAAC
GGCGCAGCCAGGTATTCGGAGCCACTACAGGTCTCCATATCTTGA.
>NM_130900.3:62-802 Homo sapiens retinoic acid early transcript
1L (RAETIL), mRNA
(SEQ ID NO: 1085)
ATGGCAGCAGCCGCCATCCCAGCTTTGCTTCTGTGCCTCCCGCTTCTGTTCCTGCTGTTCGGCT
GGTCCCGGGCTAGGCGAGACGACCCTCACTCTCTTTGCTATGACATCACCGTCATCCCTAAGTT
CAGACCTGGACCACGGTGGTGTGCGGTTCAAGGCCAGGTGGATGAAAAGACTTTTCTTCACTAT
GACTGTGGCAACAAGACAGTCACACCCGTCAGTCCCCTGGGGAAGAAACTAAATGTCACAATGG
CCTGGAAAGCACAGAACCCAGTACTGAGAGAGGTGGTGGACATACTTACAGAGCAACTGCTTGA
CATTCAGCTGGAGAATTACACACCCAAGGAACCCCTCACCCTGCAGGCAAGGATGTCTTGTGAG
CAGAAAGCTGAAGGACACAGCAGTGGATCTTGGCAGTTCAGTATCGATGGACAGACCTTCCTAC
TCTTTGACTCAGAGAAGAGAATGTGGACAACGGTTCATCCTGGAGCCAGAAAGATGAAAGAAAA
GTGGGAGAATGACAAGGATGTGGCCATGTCCTTCCATTACATCTCAATGGGAGACTGCATAGGA
TGGCTTGAGGACTTCTTGATGGGCATGGACAGCACCCTGGAGCCAAGTGCAGGAGCACCACTCG
CCATGTCCTCAGGCACAACCCAACTCAGGGCCACAGCCACCACCCTCATCCTTTGCTGCCTCCT
CATCATCCTCCCCTGCTTCATCCTCCCTGGCATCTGA.
ULBPI gene corresponds to ENSG00000111981; ULBP2 gene corresponds
to Ensembl:ENSG00000131015; ULBP3 gene corresponds to ENSG00000131019;
ULBP4 gene corresponds to ENSG00000164520; ULBP5 gene corresponds to
Ensembl (ENSG00000203722); and ULBP6 corresponds to
Ensembl:ENSG00000155918.

By “regulatory element” is meant a fragment of a nucleic acid molecule that modulates expression of a polynucleotide and/or polypeptide. In various embodiments, the regulatory element increases or decreases transcription of a gene. Non-limiting examples of regulatory elements include promoters, enhancers, silencers, and untranslated regions (UTRs).

By “persistence” in the context of an allogeneic transplant is meant the continued survival of a donor cell in a host organism. In some embodiments, allogeneic cell(s) comprising one or more of the edits described herein (e.g., a base edit in a β2M, TAP1, TAP2, Tapasin, CD58 gene, or regulatory element(s) thereof; knockdown of a β2M, TAP1, TAP2, Tapasin, and/or CD58 gene; knock-out of HLA-A, -B, and/or -C; base edit in HLA-A, -B, and/or -C; and/or overexpression of HLA-E, HLA-G, PD-L1, and/or CD47) persist in a subject allogeneic to the cells at higher levels over time post-infusion than corresponding unedited allogeneic control cells. In embodiments, the percentage of edited cells (e.g., T cells, NK cells, or lymphocytes) persisting in a subject at a given time point (e.g., 7 days, 14 days, 1 month, 3 months, 6 months, 9 months, or greater than 1, 2, or 3 years is at least about 10%, 15%, 20%, 25%, 30%, 35%, 40%, 45%, 50%, 60%, 70%, 80%, 90%, 95%, 99%, or 100% greater than the level of unedited control cells at the same time point. A cell(s) modified by methods of the present disclosure are more persistent than a reference unmodified cell(s).

By “adenine” or “9H-Purin-6-amine” is meant a purine nucleobase with the molecular formula C₅H₅N₅, having the structure

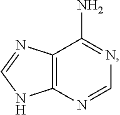

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

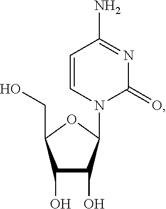

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, and PCT/US2017/045381.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 14, one of the combinations of alterations listed in Table 14, or an alteration at one or more of the amino acid positions listed in Table 14, such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1. In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject.

By “agent” is meant any small molecule chemical compound, antibody, nucleic acid molecule, or polypeptide, or fragments thereof.

“Allogeneic,” as used herein, refers to cells that are genetically dissimilar and immunologically incompatible. In embodiments, allogeneic cells are administered to a genetically dissimilar and immunologically incompatible subject. In some embodiments, the allogeneic cells comprise modifications improving their persistence in the subject allogeneic to the cells.

By “alteration” is meant a change (increase or decrease) in the level, structure, or activity of an analyte, gene or polypeptide as detected by standard art known methods such as those described herein. As used herein, an alteration includes a 10% change (e.g., increase or decrease) in expression levels. In embodiments, the increase in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Autologous,” as used herein, refers to cells from the same subject or genetically identical subject.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors include those sequences with about or at least about 85% sequence identity to any base editor sequence provided in the Sequence Listing, such as those corresponding to SEQ ID NOs:2-11.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

By “beta-2 microglobulin (β2M; B2M) polypeptide” is meant a protein having at least about 85% amino acid sequence identity to UniProt Accession No. P61769, which is provided below, or a fragment thereof having immunomodulatory activity.

>sp|P61769|B2MG_HUMAN Beta-2-microglobulin
OS = Homo sapiens OX = 9606 GN = B2M PE = 1 SV = 1
(SEQ ID NO: 455)
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDM.

By “beta-2-microglobulin (β2M; B2M) polynucleotide” is meant a nucleic acid molecule encoding an β2M polypeptide, as well as the introns, exons, 3′ untranslated regions, 5′ untranslated regions, and regulatory sequences associated with its expression, or fragments thereof. The beta-2-microglobulin gene encodes a serum protein associated with the major histocompatibility complex. β2M is involved in non-self recognition by host CD8+ T cells. An exemplary β2M polynucleotide sequence is provided at Genbank Accession No. DQ217933.1, which is provided below.

>DQ217933.1 Homo sapiens beta-2-microglobin (β2M) gene, complete cds
(SEQ ID NO: 456)
CATGTCATAAATGGTAAGTCCAAGAAAAATACAGGTATTCCCCCCCAAAGAAAACTGTAAAATC
GACTTTTTTCTATCTGTACTGTTTTTTATTGGTTTTTAAATTGGTTTTCCAAGTGAGTAAATCA
GAATCTATCTGTAATGGATTTTAAATTTAGTGTTTCTCTGTGATGTAGTAAACAAGAAACTAGA
GGCAAAAATAGCCCTGTCCCTTGCTAAACTTCTAAGGCACTTTTCTAGTACAACTCAACACTAA
CATTTCAGGCCTTTAGTGCCTTATATGAGTTTTTAAAAGGGGGAAAAGGGAGGGAGCAAGAGTG
TCTTAACTCATACATTTAGGCATAACAATTATTCTCATATTTTAGTTATTGAGAGGGCTGGTAG
AAAAACTAGGTAAATAATATTAATAATTATAGCGCTTATTAAACACTACAGAACACTTACTATG
TACCAGGCATTGTGGGAGGCTCTCTCTTGTGCATTATCTCATTTCATTAGGTCCATGGAGAGTA
TTGCATTTTCTTAGTTTAGGCATGGCCTCCACAATAAAGATTATCAAAAGCCTAAAAATATGTA
AAAGAAACCTAGAAGTTATTTGTTGTGCTCCTTGGGGAAGCTAGGCAAATCCTTTCAACTGAAA
ACCATGGTGACTTCCAAGATCTCTGCCCCTCCCCATCGCCATGGTCCACTTCCTCTTCTCACTG
TTCCTCTTAGAAAAGATCTGTGGACTCCACCACCACGAAATGGCGGCACCTTATTTATGGTCAC
TTTAGAGGGTAGGTTTTCTTAATGGGTCTGCCTGTCATGTTTAACGTCCTTGGCTGGGTCCAAG
GCAGATGCAGTCCAAACTCTCACTAAAATTGCCGAGCCCTTTGTCTTCCAGTGTCTAAAATATT
AATGTCAATGGAATCAGGCCAGAGTTTGAATTCTAGTCTCTTAGCCTTTGTTTCCCCTGTCCAT
AAAATGAATGGGGGTAATTCTTTCCTCCTACAGTTTATTTATATATTCACTAATTCATTCATTC
ATCCATCCATTCGTTCATTCGGTTTACTGAGTACCTACTATGTGCCAGCCCCTGTTCTAGGGTG
GAAACTAAGAGAATGATGTACCTAGAGGGCGCTGGAAGCTCTAAAGCCCTAGCAGTTACTGCTT
TTACTATTAGTGGTCGTTTTTTTCTCCCCCCCGCCCCCCGACAAATCAACAGAACAAAGAAAAT
TACCTAAACAGCAAGGACATAGGGAGGAACTTCTTGGCACAGAACTTTCCAAACACTTTTTCCT
GAAGGGATACAAGAAGCAAGAAAGGTACTCTTTCACTAGGACCTTCTCTGAGCTGTCCTCAGGA
TGCTTTTGGGACTATTTTTCTTACCCAGAGAATGGAGAAACCCTGCAGGGAATTCCCAAGCTGT
AGTTATAAACAGAAGTTCTCCTTCTGCTAGGTAGCATTCAAAGATCTTAATCTTCTGGGTTTCC
GTTTTCTCGAATGAAAAATGCAGGTCCGAGCAGTTAACTGGCTGGGGCACCATTAGCAAGTCAC
TTAGCATCTCTGGGGCCAGTCTGCAAAGCGAGGGGGCAGCCTTAATGTGCCTCCAGCCTGAAGT
CCTAGAATGAGCGCCCGGTGTCCCAAGCTGGGGCGCGCACCCCAGATCGGAGGGCGCCGATGTA
CAGACAGCAAACTCACCCAGTCTAGTGCATGCCTTCTTAAACATCACGAGACTCTAAGAAAAGG
AAACTGAAAACGGGAAAGTCCCTCTCTCTAACCTGGCACTGCGTCGCTGGCTTGGAGACAGGTG
ACGGTCCCTGCGGGCCTTGTCCTGATTGGCTGGGCACGCGTTTAATATAAGTGGAGGCGTCGCG
CTGGCGGGCATTCCTGAAGCTGACAGCATTCGGGCCGAGATGTCTCGCTCCGTGGCCTTAGCTG
TGCTCGCGCTACTCTCTCTTTCTGGCCTGGAGGCTATCCAGCGTGAGTCTCTCCTACCCTCCCG
CTCTGGTCCTTCCTCTCCCGCTCTGCACCCTCTGTGGCCCTCGCTGTGCTCTCTCGCTCCGTGA
CTTCCCTTCTCCAAGTTCTCCTTGGTGGCCCGCCGTGGGGCTAGTCCAGGGCTGGATCTCGGGG
AAGCGGCGGGGTGGCCTGGGAGTGGGGAAGGGGGTGCGCACCCGGGACGCGCGCTACTTGCCCC
TTTCGGCGGGGAGCAGGGGAGACCTTTGGCCTACGGCGACGGGAGGGTCGGGACAAAGTTTAGG
GCGTCGATAAGCGTCAGAGCGCCGAGGTTGGGGGAGGGTTTCTCTTCCGCTCTTTCGCGGGGCC
TCTGGCTCCCCCAGCGCAGCTGGAGTGGGGGACGGGTAGGCTCGTCCCAAAGGCGCGGCGCTGA
GGTTTGTGAACGCGTGGAGGGGCGCTTGGGGTCTGGGGGAGGCGTCGCCCGGGTAAGCCTGTCT
GCTGCGGCTCTGCTTCCCTTAGACTGGAGAGCTGTGGACTTCGTCTAGGCGCCCGCTAAGTTCG
CATGTCCTAGCACCTCTGGGTCTATGTGGGGCCACACCGTGGGGAGGAAACAGCACGCGACGTT
TGTAGAATGCTTGGCTGTGATACAAAGCGGTTTCGAATAATTAACTTATTTGTTCCCATCACAT
GTCACTTTTAAAAAATTATAAGAACTACCCGTTATTGACATCTTTCTGTGTGCCAAGGACTTTA
TGTGCTTTGCGTCATTTAATTTTGAAAACAGTTATCTTCCGCCATAGATAACTACTATGGTTAT
CTTCTGCCTCTCACAGATGAAGAAACTAAGGCACCGAGATTTTAAGAAACTTAATTACACAGGG
GATAAATGGCAGCAATCGAGATTGAAGTCAAGCCTAACCAGGGCTTTTGCGGGAGCGCATGCCT
TTTGGCTGTAATTCGTGCATTTTTTTTTAAGAAAAACGCCTGCCTTCTGCGTGAGATTCTCCAG
AGCAAACTGGGCGGCATGGGCCCTGTGGTCTTTTCGTACAGAGGGCTTCCTCTTTGGCTCTTTG
CCTGGTTGTTTCCAAGATGTACTGTGCCTCTTACTTTCGGTTTTGAAAACATGAGGGGGTTGGG
CGTGGTAGCTTACGCCTGTAATCCCAGCACTTAGGGAGGCCGAGGCGGGAGGATGGCTTGAGGT
CCGTAGTTGAGACCAGCCTGGCCAACATGGTGAAGCCTGGTCTCTACAAAAAATAATAACAAAA
ATTAGCCGGGTGTGGTGGCTCGTGCCTGTGGTCCCAGCTGCTCCGGTGGCTGAGGCGGGAGGAT
CTCTTGAGCTTAGGCTTTTGAGCTATCATGGCGCCAGTGCACTCCAGCGTGGGCAACAGAGCGA
GACCCTGTCTCTCAAAAAAGAAAAAAAAAAAAAAAGAAAGAGAAAAGAAAAGAAAGAAAGAAGT
GAAGGTTTGTCAGTCAGGGGAGCTGTAAAACCATTAATAAAGATAATCCAAGATGGTTACCAAG
ACTGTTGAGGACGCCAGAGATCTTGAGCACTTTCTAAGTACCTGGCAATACACTAAGCGCGCTC
ACCTTTTCCTCTGGCAAAACATGATCGAAAGCAGAATGTTTTGATCATGAGAAAATTGCATTTA
ATTTGAATACAATTTATTTACAACATAAAGGATAATGTATATATCACCACCATTACTGGTATTT
GCTGGTTATGTTAGATGTCATTTTAAAAAATAACAATCTGATATTTAAAAAAAAATCTTATTTT
GAAAATTTCCAAAGTAATACATGCCATGCATAGACCATTTCTGGAAGATACCACAAGAAACATG
TAATGATGATTGCCTCTGAAGGTCTATTTTCCTCCTCTGACCTGTGTGTGGGTTTTGTTTTTGT
TTTACTGTGGGCATAAATTAATTTTTCAGTTAAGTTTTGGAAGCTTAAATAACTCTCCAAAAGT
CATAAAGCCAGTAACTGGTTGAGCCCAAATTCAAACCCAGCCTGTCTGATACTTGTCCTCTTCT
TAGAAAAGATTACAGTGATGCTCTCACAAAATCTTGCCGCCTTCCCTCAAACAGAGAGTTCCAG
GCAGGATGAATCTGTGCTCTGATCCCTGAGGCATTTAATATGTTCTTATTATTAGAAGCTCAGA
TGCAAAGAGCTCTCTTAGCTTTTAATGTTATGAAAAAAATCAGGTCTTCATTAGATTCCCCAAT
CCACCTCTTGATGGGGCTAGTAGCCTTTCCTTAATGATAGGGTGTTTCTAGAGAGATATATCTG
GTCAAGGTGGCCTGGTACTCCTCCTTCTCCCCACAGCCTCCCAGACAAGGAGGAGTAGCTGCCT
TTTAGTGATCATGTACCCTGAATATAAGTGTATTTAAAAGAATTTTATACACATATATTTAGTG
TCAATCTGTATATTTAGTAGCACTAACACTTCTCTTCATTTTCAATGAAAAATATAGAGTTTAT
AATATTTTCTTCCCACTTCCCCATGGATGGTCTAGTCATGCCTCTCATTTTGGAAAGTACTGTT
TCTGAAACATTAGGCAATATATTCCCAACCTGGCTAGTTTACAGCAATCACCTGTGGATGCTAA
TTAAAACGCAAATCCCACTGTCACATGCATTACTCCATTTGATCATAATGGAAAGTATGTTCTG
TCCCATTTGCCATAGTCCTCACCTATCCCTGTTGTATTTTATCGGGTCCAACTCAACCATTTAA
GGTATTTGCCAGCTCTTGTATGCATTTAGGTTTTGTTTCTTTGTTTTTTAGCTCATGAAATTAG
GTACAAAGTCAGAGAGGGGTCTGGCATATAAAACCTCAGCAGAAATAAAGAGGTTTTGTTGTTT
GGTAAGAACATACCTTGGGTTGGTTGGGCACGGTGGCTCGTGCCTGTAATCCCAACACTTTGGG
AGGCCAAGGCAGGCTGATCACTTGAAGTTGGGAGTTCAAGACCAGCCTGGCCAACATGGTGAAA
TCCCGTCTCTACTGAAAATACAAAAATTAACCAGGCATGGTGGTGTGTGCCTGTAGTCCCAGGA
ATCACTTGAACCCAGGAGGCGGAGGTTGCAGTGAGCTGAGATCTCACCACTGCACACTGCACTC
CAGCCTGGGCAATGGAATGAGATTCCATCCCAAAAAATAAAAAAATAAAAAAATAAAGAACATA
CCTTGGGTTGATCCACTTAGGAACCTCAGATAATAACATCTGCCACGTATAGAGCAATTGCTAT
GTCCCAGGCACTCTACTAGACACTTCATACAGTTTAGAAAATCAGATGGGTGTAGATCAAGGCA
GGAGCAGGAACCAAAAAGAAAGGCATAAACATAAGAAAAAAAATGGAAGGGGTGGAAACAGAGT
ACAATAACATGAGTAATTTGATGGGGGCTATTATGAACTGAGAAATGAACTTTGAAAAGTATCT
TGGGGCCAAATCATGTAGACTCTTGAGTGATGTGTTAAGGAATGCTATGAGTGCTGAGAGGGCA
TCAGAAGTCCTTGAGAGCCTCCAGAGAAAGGCTCTTAAAAATGCAGCGCAATCTCCAGTGACAG
AAGATACTGCTAGAAATCTGCTAGAAAAAAAACAAAAAAGGCATGTATAGAGGAATTATGAGGG
AAAGATACCAAGTCACGGTTTATTCTTCAAAATGGAGGTGGCTTGTTGGGAAGGTGGAAGCTCA
TTTGGCCAGAGTGGAAATGGAATTGGGAGAAATCGATGACCAAATGTAAACACTTGGTGCCTGA
TATAGCTTGACACCAAGTTAGCCCCAAGTGAAATACCCTGGCAATATTAATGTGTCTTTTCCCG
ATATTCCTCAGGTACTCCAAAGATTCAGGTTTACTCACGTCATCCAGCAGAGAATGGAAAGTCA
AATTTCCTGAATTGCTATGTGTCTGGGTTTCATCCATCCGACATTGAAGTTGACTTACTGAAGA
ATGGAGAGAGAATTGAAAAAGTGGAGCATTCAGACTTGTCTTTCAGCAAGGACTGGTCTTTCTA
TCTCTTGTACTACACTGAATTCACCCCCACTGAAAAAGATGAGTATGCCTGCCGTGTGAACCAT
GTGACTTTGTCACAGCCCAAGATAGTTAAGTGGGGTAAGTCTTACATTCTTTTGTAAGCTGCTG
AAAGTTGTGTATGAGTAGTCATATCATAAAGCTGCTTTGATATAAAAAAGGTCTATGGCCATAC
TACCCTGAATGAGTCCCATCCCATCTGATATAAACAATCTGCATATTGGGATTGTCAGGGAATG
TTCTTAAAGATCAGATTAGTGGCACCTGCTGAGATACTGATGCACAGCATGGTTTCTGAACCAG
TAGTTTCCCTGCAGTTGAGCAGGGAGCAGCAGCAGCACTTGCACAAATACATATACACTCTTAA
CACTTCTTACCTACTGGCTTCCTCTAGCTTTTGTGGCAGCTTCAGGTATATTTAGCACTGAACG
AACATCTCAAGAAGGTATAGGCCTTTGTTTGTAAGTCCTGCTGTCCTAGCATCCTATAATCCTG
GACTTCTCCAGTACTTTCTGGCTGGATTGGTATCTGAGGCTAGTAGGAAGGGCTTGTTCCTGCT
GGGTAGCTCTAAACAATGTATTCATGGGTAGGAACAGCAGCCTATTCTGCCAGCCTTATTTCTA
ACCATTTTAGACATTTGTTAGTACATGGTATTTTAAAAGTAAAACTTAATGTCTTCCTTTTTTT
TCTCCACTGTCTTTTTCATAGATCGAGACATGTAAGCAGCATCATGGAGGTAAGTTTTTGACCT
TGAGAAAATGTTTTTGTTTCACTGTCCTGAGGACTATTTATAGACAGCTCTAACATGATAACCC
TCACTATGTGGAGAACATTGACAGAGTAACATTTTAGCAGGGAAAGAAGAATCCTACAGGGTCA
TGTTCCCTTCTCCTGTGGAGTGGCATGAAGAAGGTGTATGGCCCCAGGTATGGCCATATTACTG
ACCCTCTACAGAGAGGGCAAAGGAACTGCCAGTATGGTATTGCAGGATAAAGGCAGGTGGTTAC
CCACATTACCTGCAAGGCTTTGATCTTTCTTCTGCCATTTCCACATTGGACATCTCTGCTGAGG
AGAGAAAATGAACCACTCTTTTCCTTTGTATAATGTTGTTTTATTCTTCAGACAGAAGAGAGGA
GTTATACAGCTCTGCAGACATCCCATTCCTGTATGGGGACTGTGTTTGCCTCTTAGAGGTTCCC
AGGCCACTAGAGGAGATAAAGGGAAACAGATTGTTATAACTTGATATAATGATACTATAATAGA
TGTAACTACAAGGAGCTCCAGAAGCAAGAGAGAGGGAGGAACTTGGACTTCTCTGCATCTTTAG
TTGGAGTCCAAAGGCTTTTCAATGAAATTCTACTGCCCAGGGTACATTGATGCTGAAACCCCAT
TCAAATCTCCTGTTATATTCTAGAACAGGGAATTGATTTGGGAGAGCATCAGGAAGGTGGATGA
TCTGCCCAGTCACACTGTTAGTAAATTGTAGAGCCAGGACCTGAACTCTAATATAGTCATGTGT
TACTTAATGACGGGGACATGTTCTGAGAAATGCTTACACAAACCTAGGTGTTGTAGCCTACTAC
ACGCATAGGCTACATGGTATAGCCTATTGCTCCTAGACTACAAACCTGTACAGCCTGTTACTGT
ACTGAATACTGTGGGCAGTTGTAACACAATGGTAAGTATTTGTGTATCTAAACATAGAAGTTGC
AGTAAAAATATGCTATTTTAATCTTATGAGACCACTGTCATATATACAGTCCATCATTGACCAA
AACATCATATCAGCATTTTTTCTTCTAAGATTTTGGGAGCACCAAAGGGATACACTAACAGGAT
ATACTCTTTATAATGGGTTTGGAGAACTGTCTGCAGCTACTTCTTTTAAAAAGGTGATCTACAC
AGTAGAAATTAGACAAGTTTGGTAATGAGATCTGCAATCCAAATAAAATAAATTCATTGCTAAC
CTTTTTCTTTTCTTTTCAGGTTTGAAGATGCCGCATTTGGATTGGATGAATTCCAAATTCTGCT
TGCTTGCTTTTTAATATTGATATGCTTATACACTTACACTTTATGCACAAAATGTAGGGTTATA
ATAATGTTAACATGGACATGATCTTCTTTATAATTCTACTTTGAGTGCTGTCTCCATGTTTGAT
GTATCTGAGCAGGTTGCTCCACAGGTAGCTCTAGGAGGGCTGGCAACTTAGAGGTGGGGAGCAG
AGAATTCTCTTATCCAACATCAACATCTTGGTCAGATTTGAACTCTTCAATCTCTTGCACTCAA
AGCTTGTTAAGATAGTTAAGCGTGCATAAGTTAACTTCCAATTTACATACTCTGCTTAGAATTT
GGGGGAAAATTTAGAAATATAATTGACAGGATTATTGGAAATTTGTTATAATGAATGAAACATT
TTGTCATATAAGATTCATATTTACTTCTTATACATTTGATAAAGTAAGGCATGGTTGTGGTTAA
TCTGGTTTATTTTTGTTCCACAAGTTAAATAAATCATAAAACTTGATGTGTTATCTCTTATATC
TCACTCCCACTATTACCCCTTTATTTTCAAACAGGGAAACAGTCTTCAAGTTCCACTTGGTAAA
AAATGTGAACCCCTTGTATATAGAGTTTGGCTCACAGTGTAAAGGGCCTCAGTGATTCACATTT
TCCAGATTAGGAATCTGATGCTCAAAGAAGTTAAATGGCATAGTTGGGGTGACACAGCTGTCTA
GTGGGAGGCCAGCCTTCTATATTTTAGCCAGCGTTCTTTCCTGCGGGCCAGGTCATGAGGAGTA
TGCAGACTCTAAGAGGGAGCAAAAGTATCTGAAGGATTTAATATTTTAGCAAGGAATAGATATA
CAATCATCCCTTGGTCTCCCTGGGGGATTGGTTTCAGGACCCCTTCTTGGACACCAAATCTATG
GATATTTAAGTCCCTTCTATAAAATGGTATAGTATTTGCATATAACCTATCCACATCCTCCTGT
ATACTTTAAATCATTTCTAGATTACTTGTAATACCTAATACAATGTAAATGCTATGCAAATAGT
TGTTATTGTTTAAGGAATAATGACAAGAAAAAAAAGTCTGTACATGCTCAGTAAAGACACAACC
ATCCCTTTTTTTCCCCAGTGTTTTTGATCCATGGTTTGCTGAATCCACAGATGTGGAGCCCCTG
GATACGGAAGGCCCGCTGTACTTTGAATGACAAATAACAGATTTAAA.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “chimeric antigen receptor” or “CAR” is meant a synthetic or engineered receptor comprising an extracellular antigen binding domain joined to one or more intracellular signaling domains (e.g., T cell signaling domain) that confers specificity for an antigen onto an immune effector cell (e.g., a T-cell, an NK cell, or a macrophage). In embodiments, the CAR is a SUPRA CAR, an anti-tag CAR, a TCR-CAR, or a TCR-like CAR (see, e.g., Guedan, et al. “Engineering and Design of Chimeric Antigen Receptors,” Methods and Clinical Development, 12:145-156 (2019); Poorebrahim, et al., “TCR-like CARs and TCR-CARs targeting neoepitopes: an emerging potential,” Cancer Gene Therapy, 28:581-589 (2021); and Minutolo, et al. “The Emergence of Universal Immune Receptor T Cell Therapy for Cancer,” Front Oncol., 9:176 (2019), the disclosures of which are incorporated herein by reference in their entireties for all purposes).

By “chimeric antigen receptor (CAR) T cell” or “CAR-T cell” is meant a T cell expressing a CAR that has antigen specificity determined by the antibody-derived targeting domain of the CAR. As used herein, “CAR-T cells” includes T cells, regulatory T cells (TREG), or NK cells. As used herein, “CAR-T cells” include cells engineered to express a CAR or a T cell receptor (TCR, sometimes referred to as TCR-CARs or TCR-like CARs). Methods of making CARs (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; Poorebrahim, et al., Cancer Gene Ther 28, 581-589 (2021), https://doi.org/10.1038/s41417-021-00307-7, PCT Pubs. WO2012/079000, WO2013/059593; and U.S. Pub. 2012/0213783, the disclosure of each of which is incorporated herein by reference herein in its entirety).

By “class II, major histocompatibility complex, transactivator (CIITA) polypeptide” is meant a protein having at least about 85% amino acid sequence identity to NCBI Accession No. NP_001273331.1, which is provided below, or a fragment thereof having DNA binding activity.

>NP_001273331.1 MHC class II transactivator
isoform 1 [Homo sapiens]
(SEQ ID NO: 457)
MRCLAPRPAGSYLSEPQGSSQCATMELGPLEGGYLELLNSDADPLCLYH
FYDQMDLAGEEEIELYSEPDTDTINCDQFSRLLCDMEGDEETREAYANI
AELDQYVFQDSQLEGLSKDIFIEHIGPDEVIGESMEMPAEVGQKSQKRP
FPEELPADLKHWKPAEPPTVVTGSLLVGPVSDCSTLPCLPLPALFNOEP
ASGQMRLEKTDQIPMPFSSSSLSCLNLPEGPIQFVPTISTLPHGLWQIS
EAGTGVSSIFIYHGEVPQASQVPPPSGFTVHGLPTSPDRPGSTSPFAPS
ATDLPSMPEPALTSRANMTEHKTSPTQCPAAGEVSNKLPKWPEPVEQFY
RSLQDTYGAEPAGPDGILVEVDLVQARLERSSSKSLERELATPDWAERQ
LAQGGLAEVLLAAKEHRRPRETRVIAVLGKAGQGKSYWAGAVSRAWACG
RLPQYDFVFSVPCHCLNRPGDAYGLQDLLFSLGPQPLVAADEVFSHILK
RPDRVLLILDGFEELEAQDGFLHSTCGPAPAEPCSLRGLLAGLFQKKLL
RGCTLLLTARPRGRLVQSLSKADALFELSGFSMEQAQAYVMRYFESSGM
TEHQDRALTLLRDRPLLLSHSHSPTLCRAVCOLSEALLELGEDAKLPST
LTGLYVGLLGRAALDSPPGALAELAKLAWELGRRHQSTLQEDQFPSADV
RTWAMAKGLVQHPPRAAESELAFPSFLLQCFLGALWLALSGEIKDKELP
QYLALTPRKKRPYDNWLEGVPRFLAGLIFQPPARCLGALLGPSAAASVD
RKQKVLARYLKRLQPGTLRARQLLELLHCAHEAEEAGIWQHVVQELPGR
LSFLGTRLTPPDAHVLGKALEAAGQDFSLDLRSTGICPSGLGSLVGLSC
VTRFRAALSDTVALWESLQQHGETKLLQAAEEKFTIEPFKAKSLKDVED
LGKLVQTQRTRSSSEDTAGELPAVRDLKKLEFALGPVSGPQAFPKLVRI
LTAFSSLQHLDLDALSENKIGDEGVSQLSATFPQLKSLETLNLSQNNIT
DLGAYKLAEALPSLAASLLRLSLYNNCICDVGAESLARVLPDMVSLRVM
DVQYNKFTAAGAQQLAASLRRCPHVETLAMWTPTIPFSVQEHLQQQDSR
ISLR.

By “class II, major histocompatibililty complex, transactivator (CIITA) polynucleotide” is meant a nucleic acid molecule encoding an CIITA polypeptide, as well as the introns, exons, 3′ untranslated regions, 5′ untranslated regions, and regulatory sequences associated with its expression, or fragments thereof. An exemplary CIITA polynucleotide is provided at NCBI Accession No. NM_001286402.1, which is provide below.

>NM_001286402.1 Homo sapiens class II major histocompatibility complex
transactivator (CIITA), transcript variant 1, mRNA
(SEQ ID NO: 458)
GGTTAGTGATGAGGCTAGTGATGAGGCTGTGTGCTTCTGAGCTGGGCATCCGAAGGCATCCTTG
GGGAAGCTGAGGGCACGAGGAGGGGCTGCCAGACTCCGGGAGCTGCTGCCTGGCTGGGATTCCT
ACACAATGCGTTGCCTGGCTCCACGCCCTGCTGGGTCCTACCTGTCAGAGCCCCAAGGCAGCTC
ACAGTGTGCCACCATGGAGTTGGGGCCCCTAGAAGGTGGCTACCTGGAGCTTCTTAACAGCGAT
GCTGACCCCCTGTGCCTCTACCACTTCTATGACCAGATGGACCTGGCTGGAGAAGAAGAGATTG
AGCTCTACTCAGAACCCGACACAGACACCATCAACTGCGACCAGTTCAGCAGGCTGTTGTGTGA
CATGGAAGGTGATGAAGAGACCAGGGAGGCTTATGCCAATATCGCGGAACTGGACCAGTATGTC
TTCCAGGACTCCCAGCTGGAGGGCCTGAGCAAGGACATTTTCATAGAGCACATAGGACCAGATG
AAGTGATCGGTGAGAGTATGGAGATGCCAGCAGAAGTTGGGCAGAAAAGTCAGAAAAGACCCTT
CCCAGAGGAGCTTCCGGCAGACCTGAAGCACTGGAAGCCAGCTGAGCCCCCCACTGTGGTGACT
GGCAGTCTCCTAGTGGGACCAGTGAGCGACTGCTCCACCCTGCCCTGCCTGCCACTGCCTGCGC
TGTTCAACCAGGAGCCAGCCTCCGGCCAGATGCGCCTGGAGAAAACCGACCAGATTCCCATGCC
TTTCTCCAGTTCCTCGTTGAGCTGCCTGAATCTCCCTGAGGGACCCATCCAGTTTGTCCCCACC
ATCTCCACTCTGCCCCATGGGCTCTGGCAAATCTCTGAGGCTGGAACAGGGGTCTCCAGTATAT
TCATCTACCATGGTGAGGTGCCCCAGGCCAGCCAAGTACCCCCTCCCAGTGGATTCACTGTCCA
CGGCCTCCCAACATCTCCAGACCGGCCAGGCTCCACCAGCCCCTTCGCTCCATCAGCCACTGAC
CTGCCCAGCATGCCTGAACCTGCCCTGACCTCCCGAGCAAACATGACAGAGCACAAGACGTCCC
CCACCCAATGCCCGGCAGCTGGAGAGGTCTCCAACAAGCTTCCAAAATGGCCTGAGCCGGTGGA
GCAGTTCTACCGCTCACTGCAGGACACGTATGGTGCCGAGCCCGCAGGCCCGGATGGCATCCTA
GTGGAGGTGGATCTGGTGCAGGCCAGGCTGGAGAGGAGCAGCAGCAAGAGCCTGGAGCGGGAAC
TGGCCACCCCGGACTGGGCAGAACGGCAGCTGGCCCAAGGAGGCCTGGCTGAGGTGCTGTTGGC
TGCCAAGGAGCACCGGCGGCCGCGTGAGACACGAGTGATTGCTGTGCTGGGCAAAGCTGGTCAG
GGCAAGAGCTATTGGGCTGGGGCAGTGAGCCGGGCCTGGGCTTGTGGCCGGCTTCCCCAGTACG
ACTTTGTCTTCTCTGTCCCCTGCCATTGCTTGAACCGTCCGGGGGATGCCTATGGCCTGCAGGA
TCTGCTCTTCTCCCTGGGCCCACAGCCACTCGTGGCGGCCGATGAGGTTTTCAGCCACATCTTG
AAGAGACCTGACCGCGTTCTGCTCATCCTAGACGGCTTCGAGGAGCTGGAAGCGCAAGATGGCT
TCCTGCACAGCACGTGCGGACCGGCACCGGCGGAGCCCTGCTCCCTCCGGGGGCTGCTGGCCGG
CCTTTTCCAGAAGAAGCTGCTCCGAGGTTGCACCCTCCTCCTCACAGCCCGGCCCCGGGGCCGC
CTGGTCCAGAGCCTGAGCAAGGCCGACGCCCTATTTGAGCTGTCCGGCTTCTCCATGGAGCAGG
CCCAGGCATACGTGATGCGCTACTTTGAGAGCTCAGGGATGACAGAGCACCAAGACAGAGCCCT
GACGCTCCTCCGGGACCGGCCACTTCTTCTCAGTCACAGCCACAGCCCTACTTTGTGCCGGGCA
GTGTGCCAGCTCTCAGAGGCCCTGCTGGAGCTTGGGGAGGACGCCAAGCTGCCCTCCACGCTCA
CGGGACTCTATGTCGGCCTGCTGGGCCGTGCAGCCCTCGACAGCCCCCCCGGGGCCCTGGCAGA
GCTGGCCAAGCTGGCCTGGGAGCTGGGCCGCAGACATCAAAGTACCCTACAGGAGGACCAGTTC
CCATCCGCAGACGTGAGGACCTGGGCGATGGCCAAAGGCTTAGTCCAACACCCACCGCGGGCCG
CAGAGTCCGAGCTGGCCTTCCCCAGCTTCCTCCTGCAATGCTTCCTGGGGGCCCTGTGGCTGGC
TCTGAGTGGCGAAATCAAGGACAAGGAGCTCCCGCAGTACCTAGCATTGACCCCAAGGAAGAAG
AGGCCCTATGACAACTGGCTGGAGGGCGTGCCACGCTTTCTGGCTGGGCTGATCTTCCAGCCTC
CCGCCCGCTGCCTGGGAGCCCTACTCGGGCCATCGGCGGCTGCCTCGGTGGACAGGAAGCAGAA
GGTGCTTGCGAGGTACCTGAAGCGGCTGCAGCCGGGGACACTGCGGGCGCGGCAGCTGCTGGAG
CTGCTGCACTGCGCCCACGAGGCCGAGGAGGCTGGAATTTGGCAGCACGTGGTACAGGAGCTCC
CCGGCCGCCTCTCTTTTCTGGGCACCCGCCTCACGCCTCCTGATGCACATGTACTGGGCAAGGC
CTTGGAGGCGGCGGGCCAAGACTTCTCCCTGGACCTCCGCAGCACTGGCATTTGCCCCTCTGGA
TTGGGGAGCCTCGTGGGACTCAGCTGTGTCACCCGTTTCAGGGCTGCCTTGAGCGACACGGTGG
CGCTGTGGGAGTCCCTGCAGCAGCATGGGGAGACCAAGCTACTTCAGGCAGCAGAGGAGAAGTT
CACCATCGAGCCTTTCAAAGCCAAGTCCCTGAAGGATGTGGAAGACCTGGGAAAGCTTGTGCAG
ACTCAGAGGACGAGAAGTTCCTCGGAAGACACAGCTGGGGAGCTCCCTGCTGTTCGGGACCTAA
AGAAACTGGAGTTTGCGCTGGGCCCTGTCTCAGGCCCCCAGGCTTTCCCCAAACTGGTGCGGAT
CCTCACGGCCTTTTCCTCCCTGCAGCATCTGGACCTGGATGCGCTGAGTGAGAACAAGATCGGG
GACGAGGGTGTCTCGCAGCTCTCAGCCACCTTCCCCCAGCTGAAGTCCTTGGAAACCCTCAATC
TGTCCCAGAACAACATCACTGACCTGGGTGCCTACAAACTCGCCGAGGCCCTGCCTTCGCTCGC
TGCATCCCTGCTCAGGCTAAGCTTGTACAATAACTGCATCTGCGACGTGGGAGCCGAGAGCTTG
GCTCGTGTGCTTCCGGACATGGTGTCCCTCCGGGTGATGGACGTCCAGTACAACAAGTTCACGG
CTGCCGGGGCCCAGCAGCTCGCTGCCAGCCTTCGGAGGTGTCCTCATGTGGAGACGCTGGCGAT
GTGGACGCCCACCATCCCATTCAGTGTCCAGGAACACCTGCAACAACAGGATTCACGGATCAGC
CTGAGATGATCCCAGCTGTGCTCTGGACAGGCATGTTCTCTGAGGACACTAACCACGCTGGACC
TTGAACTGGGTACTTGTGGACACAGCTCTTCTCCAGGCTGTATCCCATGAGCCTCAGCATCCTG
GCACCCGGCCCCTGCTGGTTCAGGGTTGGCCCCTGCCCGGCTGCGGAATGAACCACATCTTGCT
CTGCTGACAGACACAGGCCCGGCTCCAGGCTCCTTTAGCGCCCAGTTGGGTGGATGCCTGGTGG
CAGCTGCGGTCCACCCAGGAGCCCCGAGGCCTTCTCTGAAGGACATTGCGGACAGCCACGGCCA
GGCCAGAGGGAGTGACAGAGGCAGCCCCATTCTGCCTGCCCAGGCCCCTGCCACCCTGGGGAGA
AAGTACTTCTTTTTTTTTATTTTTAGACAGAGTCTCACTGTTGCCCAGGCTGGCGTGCAGTGGT
GCGATCTGGGTTCACTGCAACCTCCGCCTCTTGGGTTCAAGCGATTCTTCTGCTTCAGCCTCCC
GAGTAGCTGGGACTACAGGCACCCACCATCATGTCTGGCTAATTTTTCATTTTTAGTAGAGACA
GGGTTTTGCCATGTTGGCCAGGCTGGTCTCAAACTCTTGACCTCAGGTGATCCACCCACCTCAG
CCTCCCAAAGTGCTGGGATTACAAGCGTGAGCCACTGCACCGGGCCACAGAGAAAGTACTTCTC
CACCCTGCTCTCCGACCAGACACCTTGACAGGGCACACCGGGCACTCAGAAGACACTGATGGGC
AACCCCCAGCCTGCTAATTCCCCAGATTGCAACAGGCTGGGCTTCAGTGGCAGCTGCTTTTGTC
TATGGGACTCAATGCACTGACATTGTTGGCCAAAGCCAAAGCTAGGCCTGGCCAGATGCACCAG
CCCTTAGCAGGGAAACAGCTAATGGGACACTAATGGGGCGGTGAGAGGGGAACAGACTGGAAGC
ACAGCTTCATTTCCTGTGTCTTTTTTCACTACATTATAAATGTCTCTTTAATGTCACAGGCAGG
TCCAGGGTTTGAGTTCATACCCTGTTACCATTTTGGGGTACCCACTGCTCTGGTTATCTAATAT
GTAACAAGCCACCCCAAATCATAGTGGCTTAAAACAACACTCACATTTA.

By “Cluster of Differentiation 47 (CD47) polypeptide” is meant a protein having at least about 85% amino acid sequence identity to NCBI Accession No. NP_001768.1, which is provided below, or a fragment thereof having immunomodulatory activity.

| (SEQ ID NO: 459) | |
|---|---|
| 1 | mwplvaalll gsaccgsaql lfnktksvef tfcndtvvip cfvtnmeaqn ttevyvkwkf |
| 61 | kgrdiytfdg alnkstvptd fssakievsq llkgdaslkm dksdavshtg nytcevtelt |
| 121 | regetiielk yrvvswfspn enilivifpi faillfwgqf giktlkyrsg gmdektiall |
| 181 | vaglvitviv ivgailfvpg eyslknatgl glivtstgil illhyyvfst aigltsfvia |
| 241 | ilviqviayi lavvglslci aacipmhgpl lisglsilal aqllglvymk fvasnaktiq |
| 301 | pprkaveepl nafkeskgmm nde. |

By “Cluster of Differentiation 47 (CD47) polynucleotide” is meant a nucleic acid molecule encoding an CD47 polypeptide, as well as the introns, exons, 3′ untranslated regions, 5′ untranslated regions, and regulatory sequences associated with its expression, or fragments thereof. An exemplary CD47 polynucleotide is provided at NCBI Accession No. NM_001777.4, which is provided below.

| (SEQ ID NO: 460) | |
|---|---|
| 1 | gcagcctggg cagtgggtcc tgcctgtgac gcgcggcggc ggtcggtcct gcctgtaacg |
| 61 | gcggcggcgg ctgctgctcc ggacacctgc ggcggcggcg gcgaccccgc ggcgggcgcg |
| 121 | gagatgtggc ccctggtagc ggcgctgttg ctgggctcgg cgtgctgcgg atcagctcag |
| 181 | ctactattta ataaaacaaa atctgtagaa ttcacgtttt gtaatgacac tgtcgtcatt |
| 241 | ccatgctttg ttactaatat ggaggcacaa aacactactg aagtatacgt aaagtggaaa |
| 301 | tttaaaggaa gagatattta cacctttgat ggagctctaa acaagtccac tgtccccact |
| 361 | gactttagta gtgcaaaaat tgaagtctca caattactaa aaggagatgc ctctttgaag |
| 421 | atggataaga gtgatgctgt ctcacacaca ggaaactaca cttgtgaagt aacagaatta |
| 481 | accagagaag gtgaaacgat catcgagcta aaatatcgtg ttgtttcatg gttttctcca |
| 541 | aatgaaaata ttcttattgt tattttccca atttttgcta tactcctgtt ctggggacag |
| 601 | tttggtatta aaacacttaa atatagatcc ggtggtatgg atgagaaaac aattgcttta |
| 661 | cttgttgctg gactagtgat cactgtcatt gtcattgttg gagccattct tttcgtccca |
| 721 | ggtgaatatt cattaaagaa tgctactggc cttggtttaa ttgtgacttc tacagggata |
| 781 | ttaatattac ttcactacta tgtgtttagt acagcgattg gattaacctc cttcgtcatt |
| 841 | gccatattgg ttattcaggt gatagcctat atcctcgctg tggttggact gagtctctgt |
| 901 | attgcggcgt gtataccaat gcatggccct cttctgattt caggtttgag tatcttagct |
| 961 | ctagcacaat tacttggact agtttatatg aaatttgtgg cttccaatca gaagactata |
| 1021 | caacctccta ggaaagctgt agaggaaccc cttaatgcat tcaaagaatc aaaaggaatg |
| 1081 | atgaatgatg aataactgaa gtgaagtgat ggactccgat ttggagagta gtaagacgtg |
| 1141 | aaaggaatac acttgtgttt aagcaccatg gccttgatga ttcactgttg gggagaagaa |
| 1201 | acaagaaaag taactggttg tcacctatga gacccttacg tgattgttag ttaagttttt |
| 1261 | attcaaagca gctgtaattt agttaataaa ataattatga tctatgttgt ttgcccaatt |
| 1321 | gagatccagt tttttgttgt tatttttaat caattagggg caatagtaga atggacaatt |
| 1381 | tccaagaatg atgcctttca ggtcctaggg cctctggcct ctaggtaacc agtttaaatt |
| 1441 | ggttcagggt gataactact tagcactgcc ctggtgatta cccagagata tctatgaaaa |
| 1501 | ccagtggctt ccatcaaacc tttgccaact caggttcaca gcagctttgg gcagttatgg |
| 1561 | cagtatggca ttagctgaga ggtgtctgcc acttctgggt caatggaata ataaattaag |
| 1621 | tacaggcagg aatttggttg ggagcatctt gtatgatctc cgtatgatgt gatattgatg |
| 1681 | gagatagtgg tcctcattct tgggggttgc cattcccaca ttcccccttc aacaaacagt |
| 1741 | gtaacaggtc cttcccagat ttagggtact tttattgatg gatatgtttt ccttttattc |
| 1801 | acataacccc ttgaaaccct gtcttgtcct cctgttactt gcttctgctg tacaagatgt |
| 1861 | agcacctttt ctcctctttg aacatggtct agtgacacgg tagcaccagt tgcaggaagg |
| 1921 | agccagactt gttctcagag cactgtgttc acacttttca gcaaaaatag ctatggttgt |
| 1981 | aacatatgta ttcccttcct ctgatttgaa ggcaaaaatc tacagtgttt cttcacttct |
| 2041 | tttctgatct ggggcatgaa aaaagcaaga ttgaaatttg aactatgagt ctcctgcatg |
| 2101 | gcaacaaaat gtgtgtcacc atcaggccaa caggccagcc cttgaatggg gatttattac |
| 2161 | tgttgtatct atgttgcatg ataaacattc atcaccttcc tcctgtagtc ctgcctcgta |
| 2221 | ctccccttcc cctatgattg aaaagtaaac aaaacccaca tttcctatcc tggttagaag |
| 2281 | aaaattaatg ttctgacagt tgtgatcgcc tggagtactt ttagactttt agcattcgtt |
| 2341 | ttttacctgt ttgtggatgt gtgtttgtat gtgcatacgt atgagatagg cacatgcatc |
| 2401 | ttctgtatgg acaaaggtgg ggtacctaca ggagagcaaa ggttaatttt gtgcttttag |
| 2461 | taaaaacatt taaatacaaa gttctttatt gggtggaatt atatttgatg caaatatttg |
| 2521 | atcacttaaa acttttaaaa cttctaggta atttgccacg ctttttgact gctcaccaat |
| 2581 | accctgtaaa aatacgtaat tcttcctgtt tgtgtaataa gatattcata tttgtagttg |
| 2641 | cattaataat agttatttct tagtccatca gatgttcccg tgtgcctctt ttatgccaaa |
| 2701 | ttgattgtca tatttcatgt tgggaccaag tagtttgccc atggcaaacc taaatttatg |
| 2761 | acctgctgag gcctctcaga aaactgagca tactagcaag acagctcttc ttgaaaaaaa |
| 2821 | aaatatgtat acacaaatat atacgtatat ctatatatac gtatgtatat acacacatgt |
| 2881 | atattcttcc ttgattgtgt agctgtccaa aataataaca tatatagagg gagctgtatt |
| 2941 | cctttataca aatctgatgg ctcctgcagc actttttcct tctgaaaata tttacatttt |
| 3001 | gctaacctag tttgttactt taaaaatcag ttttgatgaa aggagggaaa agcagatgga |
| 3061 | cttgaaaaag atccaagctc ctattagaaa aggtatgaaa atctttatag taaaattttt |
| 3121 | tataaactaa agttgtacct tttaatatgt agtaaactct catttatttg gggttcgctc |
| 3181 | ttggatctca tccatccatt gtgttctctt taatgctgcc tgccttttga ggcattcact |
| 3241 | gccctagaca atgccaccag agatagtggg ggaaatgcca gatgaaacca actcttgctc |
| 3301 | tcactagttg tcagcttctc tggataagtg accacagaag caggagtcct cctgcttggg |
| 3361 | catcattggg ccagttcctt ctctttaaat cagatttgta atggctccca aattccatca |
| 3421 | catcacattt aaattgcaga cagtgttttg cacatcatgt atctgttttg tcccataata |
| 3481 | tgctttttac tccctgatcc cagtttctgc tgttgactct tccattcagt tttatttatt |
| 3541 | gtgtgttctc acagtgacac catttgtcct tttctgcaac aacctttcca gctacttttg |
| 3601 | ccaaattcta tttgtcttct ccttcaaaac attctccttt gcagttcctc ttcatctgtg |
| 3661 | tagctgctct tttgtctctt aacttaccat tcctatagta ctttatgcat ctctgcttag |
| 3721 | ttctattagt tttttggcct tgctcttctc cttgatttta aaattccttc tatagctaga |
| 3781 | gcttttcttt ctttcattct ctcttcctgc agtgttttgc atacatcaga agctaggtac |
| 3841 | ataagttaaa tgattgagag ttggctgtat ttagatttat cactttttaa tagggtgagc |
| 3901 | ttgagagttt tctttctttc tgtttttttt ttttgttttt tttttttttt tttttttttt |
| 3961 | tttttttgac taatttcaca tgctctaaaa accttcaaag gtgattattt ttctcctgga |
| 4021 | aactccaggt ccattctgtt taaatcccta agaatgtcag aattaaaata acagggctat |
| 4081 | cccgtaattg gaaatatttc ttttttcagg atgctatagt caatttagta agtgaccacc |
| 4141 | aaattgttat ttgcactaac aaagctcaaa acacgataag tttactcctc catctcagta |
| 4201 | ataaaaatta agctgtaatc aaccttctag gtttctcttg tcttaaaatg ggtattcaaa |
| 4261 | aatggggatc tgtggtgtat gtatggaaac acatactcct taatttacct gttgttggaa |
| 4321 | actggagaaa tgattgtcgg gcaaccgttt attttttatt gtattttatt tggttgaggg |
| 4381 | atttttttat aaacagtttt acttgtgtca tattttaaaa ttactaactg ccatcacctg |
| 4441 | ctggggtcct ttgttaggtc attttcagtg actaataggg ataatccagg taactttgaa |
| 4501 | gagatgagca gtgagtgacc aggcagtttt tctgccttta gctttgacag ttcttaatta |
| 4561 | agatcattga agaccagctt tctcataaat ttctcttttt gaaaaaaaga aagcatttgt |
| 4621 | actaagctcc tctgtaagac aacatcttaa atcttaaaag tgttgttatc atgactggtg |
| 4681 | agagaagaaa acattttgtt tttattaaat ggagcattat ttacaaaaag ccattgttga |
| 4741 | gaattagatc ccacatcgta taaatatcta ttaaccatto taaataaaga gaactccagt |
| 4801 | gttgctatgt gcaagatcct ctcttggagc ttttttgcat agcaattaaa ggtgtgctat |
| 4861 | ttgtcagtag ccattttttt gcagtgattt gaagaccaaa gttgttttac agctgtgtta |
| 4921 | ccgttaaagg tttttttttt tatatgtatt aaatcaattt atcactgttt aaagctttga |
| 4981 | atatctgcaa tctttgccaa ggtacttttt tatttaaaaa aaaacataac tttgtaaata |
| 5041 | ttaccctgta atattatata tacttaataa aacattttaa gctattttgt tgggctattt |
| 5101 | ctattgctgc tacagcagac cacaagcaca tttctgaaaa atttaattta ttaatgtatt |
| 5161 | tttaagttgc ttatattcta ggtaacaatg taaagaatga tttaaaatat taattatgaa |
| 5221 | ttttttgagt ataataccca ataagctttt aattagagca gagttttaat taaaagtttt |
| 5281 | aaatcagtcc aa. The CD47 gene corresponds to ENSG00000196776. |

By “Cluster of Differentiation 58 (CD58) polypeptide” is meant a protein having at least about 85% amino acid sequence identity to NCBI Reference Sequence Accession No. NP_001770.1, which is provided below, or a fragment thereof that functions in the immune system. CD58 and the Immunobiology thereof is described in Zhang, et al. “CD58 Immunobiology at a Glance,” Frontiers in Immunology, vol. 12, article 705260 (2021), the disclosure of which is incorporated herein by reference in its entirety for all purposes.

| (SEQ ID NO: 461) | |
|---|---|
| 1 | MVAGSDAGRA LGVLSVVCLL HCFGFISCFS QQIYGVVYGN VTFHVPSNVP LKEVLWKKQK |
| 61 | DKVAELENSE FRAFSSFKNR VYLDTVSGSL TIYNLTSSDE DEYEMESPNI TDTMKFFLYV |
| 121 | LESLPSPILT CALINGSIEV QCMIPEHYNS HRGLIMYSWD CPMEQCKRNS TSIYFKMEND |
| 181 | LPQKIQCTLS NPLFNTTSSI ILTTCIPSSG HSRHRYALIP IPLAVITTCI VLYMNGILKC |
| 241 | DRKPDRTNSN |

By “Cluster of Differentiation 58 (CD58) polynucleotide” is meant a nucleic acid molecule encoding an CD58 polypeptide, as well as the introns, exons, 3′ untranslated regions, 5′ untranslated regions, and regulatory sequences associated with its expression, or fragments thereof. An exemplary CD58 polynucleotide is provided at NCBI Accession No. NM_001779.3, which is reproduced below.

(SEQ ID NO: 462)
ATGGTTGCTGGGAGCGACGCGGGGGGGGCCCTGGGGGTCCTCAGCGTGG
TCTGCCTGCTGCACTGCTTTGGTTTCATCAGCTGTTTTTCCCAACAAAT
ATATGGTGTTGTGTATGGGAATGTAACTTTCCATGTACCAAGCAATGTG
CCTTTAAAAGAGGTCCTATGGAAAAAACAAAAGGATAAAGTTGCAGAAC
TGGAAAATTCTGAATTCAGAGCTTTCTCATCTTTTAAAAATAGGGTTTA
TTTAGACACTGTGTCAGGTAGCCTCACTATCTACAACTTAACATCATCA
GATGAAGATGAGTATGAAATGGAATCGCCAAATATTACTGATACCATGA
AGTTCTTTCTTTATGTGCTTGAGTCTCTTCCATCTCCCACACTAACTTG
TGCATTGACTAATGGAAGCATTGAAGTCCAATGCATGATACCAGAGCAT
TACAACAGCCATCGAGGACTTATAATGTACTCATGGGATTGTCCTATGG
AGCAATGTAAACGTAACTCAACCAGTATATATTTTAAGATGGAAAATGA
TCTTCCACAAAAAATACAGTGTACTCTTAGCAATCCATTATTTAATACA
ACATCATCAATCATTTTGACAACCTGTATCCCAAGCAGCGGTCATTCAA
GACACAGATATGCACTTATACCCATACCATTAGCAGTAATTACAACATG
TATTGTGCTGTATATGAATGGTATTCTGAAATGTGACAGAAAACCAGAC
AGAACCAACTCCAATTGA.
The CD58 gene corresponds to Ensembl:
ENSG00000116815.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following:

| | |
|---|---|
| Glutamine | CAG → TAG Stop codon |
| | CAA → TAA |
| Arginine | CGA → TGA |
| Tryptophan | TGG → TGA |
| | TGG → TAG |
| | TGG → TAA |

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and 7r-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

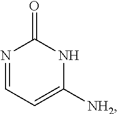

and corresponding to CAS No. 71-30-7.

By “cytidine” is meant a cytosine molecule attached to a ribose sugar via a glycosidic bond, having the structure

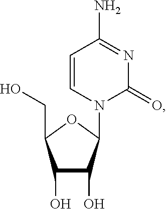

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of catalyzing a deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. PmCDA1 (SEQ ID NO: 13-14), which is derived from Petromyzon marinus (Petromyzon marinus cytosine deaminase 1, “PmCDA1”), AID (Activation-induced cytidine deaminase; AICDA) (Exemplary AID polypeptide sequences are provided in the Sequence Listing as SEQ ID NOs: 15-21), which is derived from a mammal (e.g., human, swine, bovine, horse, monkey etc.), and APOBEC are exemplary cytidine deaminases (Exemplary APOBEC polypeptide sequences are provided in the Sequence Listing as SEQ ID NOs: 12-61. Further exemplary cytidine deaminase (CDA) sequences are provided in the Sequence Listing as SEQ ID NOs: 62-66. Additional exemplary cytidine deaminase sequences, including APOBEC polypeptide sequences, are provided in the Sequence Listing as SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344.

By “cytosine” is meant a pyrimidine nucleobase with the molecular formula C₄H₅N₃O.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. In some embodiments, the disease is hematological cancer or solid tumors.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity.

By “effective amount” is meant the amount of an agent or active compound, a modified immune cell, or a base editor as described herein, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the present disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is the amount of a modified immune cell (e.g., T- or NK-cell) required to achieve a therapeutic effect (e.g., reduce or stabilize cancer cell proliferation, tumor burden, or cancer cell survival). In one embodiment, an effective amount is sufficient to ameliorate one or more symptoms of a disease.

The term “exonuclease” refers to a protein or polypeptide capable of removing successive nucleotides from either the 5′ or 3′ end of a polynucleotide (e.g., RNA or DNA).

The term “endonuclease” refers to a protein or polypeptide capable of catalyzing the cleavage of internal regions in a nucleic acid molecule (e.g., DNA or RNA).

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

In general, a “gene” is a region on the genome that is capable of being transcribed to an RNA that either has a regulatory function, a catalytic function, and/or encodes a protein. An eukaryotic gene typically has introns and exons, which may organize to produce different RNA splice variants that encode alternative versions of a mature protein. The skilled artisan will appreciate that the present disclosure encompasses all transcripts encoding a polypeptide of interest, including splice variants, allelic variants and transcripts that occur because of alternative promoter sites or alternative poly-adenylation sites. A “full-length” gene or RNA therefore encompasses any naturally occurring splice variants, allelic variants, other alternative transcripts, splice variants generated by recombinant technologies which bear the same function as the naturally occurring variants, and the resulting RNA molecules. In some embodiments, the fragment is a functional fragment.

“Graft versus host disease” (GVHD) refers to a pathological condition where transplanted cells of a donor generate an immune response against cells of the host.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule. In some embodiments the guide polynucleotide is selected from Table 1 or Table 1B.

“Host versus graft disease” (HVGD) or “host-versus-graft rejection” refers to a pathological condition where the immune system of a host generates an immune response against transplanted cells of an allogeneic donor.

By “Human Leukocyte Antigen-E (HLA-E) polypeptide” is meant a protein having at least about 85% amino acid sequence identity to NCBI Accession No. NP_005507.3, or a fragment thereof having immunomodulatory activity. An exemplary amino acid sequence is provided below.

| (SEQ ID NO: 463) | |
|---|---|
| 1 | mvdgtlllll sealaltqtw agshslkyfh tsvsrpgrge prfisvgyvd dtqfvrfdnd |
| 61 | aasprmvpra pwmegegsey wdretrsard taqifrvnlr tlrgyynqse agshtlqwmh |
| 121 | gcelgpdgrf lrgyeqfayd gkdyltlned lrswtavdta aqiseqksnd aseaehqray |
| 181 | ledtcvewlh kylekgketl lhleppkthv thhpisdhea tlrcwalgfy paeitltwqq |
| 241 | dgeghtqdte lvetrpagdg tfqkwaavvv psgeeqrytc hvqheglpep vtlrwkpasq |
| 301 | ptipivgiia glvllgsvvs gavvaaviwr kkssggkggs yskaewsdsa qgseshsl. |

By “Human Leukocyte Antigen-E (HLA-E) polynucleotide” is meant a nucleic acid molecule encoding an HLA-E polypeptide, as well as the introns, exons, 3′ untranslated regions, 5′ untranslated regions, and regulatory sequences associated with its expression, or fragments thereof. An exemplary HLA-E polynucleotide is provided at NCBI Accession No. NM_005516.6, which is provided below.

| (SEQ ID NO: 464) | |
|---|---|
| 1 | ctcaggactc agaggctggg atcatggtag atggaaccct ccttttactc ctctcggagg |
| 61 | ccctggccct tacccagacc tgggcgggct cccactcctt gaagtatttc cacacttccg |
| 121 | tgtcccggcc cggccgcggg gagccccgct tcatctctgt gggctacgtg gacgacaccc |
| 181 | agttcgtgcg cttcgacaac gacgccgcga gtccgaggat ggtgccgcgg gcgccgtgga |
| 241 | tggagcagga ggggtcagag tattgggacc gggagacacg gagcgccagg gacaccgcac |
| 301 | agattttccg agtgaatctg cggacgctgc gcggctacta caatcagagc gaggccgggt |
| 361 | ctcacaccct gcagtggatg catggctgcg agctggggcc cgacgggcgc ttcctccgcg |
| 421 | ggtatgaaca gttcgcctac gacggcaagg attatctcac cctgaatgag gacctgcgct |
| 481 | cctggaccgc ggtggacacg gcggctcaga tctccgagca aaagtcaaat gatgcctctg |
| 541 | aggcggagca ccagagagcc tacctggaag acacatgcgt ggagtggctc cacaaatacc |
| 601 | tggagaaggg gaaggagacg ctgcttcacc tggagccccc aaagacacac gtgactcacc |
| 661 | accccatctc tgaccatgag gccaccctga ggtgctgggc cctgggcttc taccctgcgg |
| 721 | agatcacact gacctggcag caggatgggg agggccatac ccaggacacg gagctcgtgg |
| 781 | agaccaggcc tgcaggggat ggaaccttcc agaagtgggc agctgtggtg gtgccttctg |
| 841 | gagaggagca gagatacacg tgccatgtgc agcatgaggg gctacccgag cccgtcaccc |
| 901 | tgagatggaa gccggcttcc cagcccacca tccccatcgt gggcatcatt gctggcctgg |
| 961 | ttctccttgg atctgtggtc tctggagctg tggttgctgc tgtgatatgg aggaagaaga |
| 1021 | gctcaggtgg aaaaggaggg agctactcta aggctgagtg gagcgacagt gcccaggggt |
| 1081 | ctgagtctca cagcttgtaa agcctgagac agctgccttg tgtgcgactg agatgcacag |
| 1141 | ctgccttgtg tgcgactgag atgcaggatt tcctcacgcc tcccctatgt gtcttagggg |
| 1201 | actctggctt ctctttttgc aagggcctct gaatctgtct gtgtccctgt tagcacaatg |
| 1261 | tgaggaggta gagaaacagt ccacctctgt gtctaccatg acccccttcc tcacactgac |
| 1321 | ctgtgttcct tccctgttct cttttctatt aaaaataaga acctgggcag agtgcggcag |
| 1381 | ctcatgcctg taatcccagc acttagggag gccgaggagg gcagatcacg aggtcaggag |
| 1441 | atcgaaacca tcctggctaa cacggtgaaa ccccgtctct actaaaaaat acaaaaaatt |
| 1501 | agctgggcgc agaggcacgg gcctgtagtc ccagctactc aggaggcgga ggcaggagaa |
| 1561 | tggcgtcaac ccgggaggcg gaggttgcag tgagccagga ttgtgcgact gcactccagc |
| 1621 | ctgggtgaca gggtgaaacg ccatctcaaa aaataaaaat tgaaaaataa aaaaagaacc |
| 1681 | tggatctcaa tttaattttt catattcttg caatgaaatg gacttgagga agctaagatc |
| 1741 | atagctagaa atacagataa ttccacagca catctctagc aaatttagcc tattcctatt |
| 1801 | ctctagccta ttccttacca cctgtaatct tgaccatata ccttggagtt gaatattgtt |
| 1861 | ttcatactgc tgtggtttga atgttccctc caacactcat gttgagactt aatccctaat |
| 1921 | gtggcaatac tgaaaggtgg ggcctttgag atgtgattgg atcgtaaggc tgtgccttca |
| 1981 | ttcatgggtt aatggattaa tgggttatca caggaatggg actggtggct ttataagaag |
| 2041 | aggaaaagag aactgagcta gcatgcccag cccacagaga gcctccacta gagtgatgct |
| 2101 | aagtggaaat gtgaggtgca gctgccacag agggccccca ccagggaaat gtctagtgtc |
| 2161 | tagtggatcc aggccacagg agagagtgcc ttgtggagcg ctgggagcag gacctgacca |
| 2221 | ccaccaggac cccagaactg tggagtcagt ggcagcatgc agcgccccct tgggaaagct |
| 2281 | ttaggcacca gcctgcaacc cattcgagca gccacgtagg ctgcacccag caaagccaca |
| 2341 | ggcacggggc tacctgaggc cttgggggcc caatccctgc tccagtgtgt ccgtgaggca |
| 2401 | gcacacgaag tcaaaagaga ttattctctt cccacagata ccttttctct cccatgaccc |
| 2461 | tttaacagca tctgcttcat tcccctcacc ttcccaggct gatctgaggt aaactttgaa |
| 2521 | gtaaaataaa agctgtgttt gagcatca. |

The HLA-E gene corresponds to Ensembl: ENSG00000116815.

By “Human Leukocyte Antigen-G (HLA-G) polypeptide” is meant a protein having at least about 85% amino acid sequence identity to NCBI Accession No. NP_001350496.1, which is provided below, or a fragment thereof having immunomodulatory activity.

| (SEQ ID NO: 465) | |
|---|---|
| 1 | mktprmvvma prtlflllsg altltetwag shsmryfsaa vsrpgrgepr fiamgyvddt |
| 61 | qfvrfdsdsa cprmeprapw veqegpeywe eetrntkaha qtdrmnlqtl rgyynqseas |
| 121 | shtlqwmigc dlgsdgrllr gyeqyaydgk dylalnedlr swtaadtaaq iskrkceaan |
| 181 | vaeqrrayle gtcvewlhry lengkemlqr adppkthvth hpvfdyeatl rcwalgfypa |
| 241 | eiiltwqrdg edqtqdvelv etrpagdgtf qkwaavvvps geeqrytchv qheglpeplm |
| 301 | lrwkqsslpt ipimgivagl vvlaavvtga avaavlwrkk ssd |

By “Human Leukocyte Antigen-G (HLA-G) polynucleotide” is meant a nucleic acid molecule encoding an HLA-G polypeptide, as well as the introns, exons, 3′ untranslated regions, 5′ untranslated regions, and regulatory sequences associated with its expression, or fragments thereof. An exemplary HLA-G polynucleotide is provided at NCBI Accession No. NM_001363567.2, which is provided below.

| (SEQ ID NO: 466) | |
|---|---|
| 1 | atatagtaac atagtgtggt actttgtctt gaggagatgt cctggactca cacggaaact |
| 61 | tagggctacg gaatgaagac gccaaggatg gtggtcatgg cgccccgaac cctcttcctg |
| 121 | ctgctctcgg gggccctgac cctgaccgag acctgggcgg gctcccactc catgaggtat |
| 181 | ttcagcgccg ccgtgtcccg gcccggccgc ggggagcccc gcttcatcgc catgggctac |
| 241 | gtggacgaca cgcagttcgt gcggttcgac agcgactcgg cgtgtccgag gatggagccg |
| 301 | cgggcgccgt gggtggagca ggaggggccg gagtattggg aagaggagac acggaacacc |
| 361 | aaggcccacg cacagactga cagaatgaac ctgcagaccc tgcgcggcta ctacaaccag |
| 421 | agcgaggcca gttctcacac cctccagtgg atgattggct gcgacctggg gtccgacgga |
| 481 | cgcctcctcc gcgggtatga acagtatgcc tacgatggca aggattacct cgccctgaac |
| 541 | gaggacctgc gctcctggac cgcagcggac actgcggctc agatctccaa gcgcaagtgt |
| 601 | gaggcggcca atgtggctga acaaaggaga gcctacctgg agggcacgtg cgtggagtgg |
| 661 | ctccacagat acctggagaa cgggaaggag atgctgcagc gcgcggaccc ccccaagaca |
| 721 | cacgtgaccc accaccctgt ctttgactat gaggccaccc tgaggtgctg ggccctgggc |
| 781 | ttctaccctg cggagatcat actgacctgg cagcgggatg gggaggacca gacccaggac |
| 841 | gtggagctcg tggagaccag gcctgcaggg gatggaacct tccagaagtg ggcagctgtg |
| 901 | gtggtgcctt ctggagagga gcagagatac acgtgccatg tgcagcatga ggggctgccg |
| 961 | gagcccctca tgctgagatg gaagcagtct tccctgccca ccatccccat catgggtatc |
| 1021 | gttgctggcc tggttgtcct tgcagctgta gtcactggag ctgcggtcgc tgctgtgctg |
| 1081 | tggagaaaga agagctcaga ttgaaaagga gggagctact ctcaggctgc aatgtgaaac |
| 1141 | agctgccctg tgtgggactg agtggcaagt ccctttgtga cttcaagaac cctgactcct |
| 1201 | ctttgtgcag agaccagccc acccctgtgc ccaccatgac cctcttcctc atgctgaact |
| 1261 | gcattccttc cccaatcacc tttcctgttc cagaaaaggg gctgggatgt ctccgtctct |
| 1321 | gtctcaaatt tgtggtccac tgagctataa cttacttctg tattaaaatt agaatctgag |
| 1381 | tataa. |

The HLA-G gene corresponds to ENSG00000230413, ENSG00000233095,
ENSG00000237216, ENSG00000276051 and ENGS00000204632.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immune cell” is meant a cell of the immune system capable of generating an immune response. Exemplary immune cell include, but are not limited to, T cells, NK cells, B cells, macrophages, hematopoietic stem cells, or precursors thereof. In embodiments, an immune cell is allogeneic to a subject to whom the cell is to be administered. In embodiments, an immune cell is from a donor and is allogeneic to a subject to which the immune cell will be administered after being modified according to the methods provided herein. The invention of the disclosure features methods for preparing modified allogeneic immune cells with improved characteristics (e.g., increased persistence in a subject) as well as the cells produced by these methods.

By “immune effector cell” is meant a lymphocyte, once activated, capable of effecting an immune response upon a target cell. In some embodiments, immune effector cells are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, immune effector cells are effector NK cells. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell).

By “immune response regulation polypeptide” is meant a protein that modulates an immune response. An immune response regulation polypeptide may directly or indirectly modulate an immune response. For example, an immune response regulation polypeptide may increase or decrease the activation of an immune cell, e.g. a T cell, NK cell. An immune response regulation polypeptide may increase or decrease the activation threshold of an immune cell. In some embodiments, the immune response regulation polypeptide modulates a signal

By “immune response regulation polynucleotide” or “immune response regulator polynucleotide” is meant a nucleic acid molecule that encodes a polypeptide that modulates an immune response.

By “immunogen” is meant a polypeptide or fragment thereof capable of inducing an immune response. Exemplary immunogens include CD2, CD3e, CD3 delta, CD3 gamma, TRAC, TRBC1, TRBC2, CD4, CD5, CD7, CD8, CD19, CD23, CD27, CD28, CD30, CD33, CD52, CD58, CD70, CD127, CD122, CD130, CD132, CD38, CD69, CD11a, CD58, CD99, CD103, CCR4, CCR5, CCR6, CCR9, CCR10, CXCR3, CXCR4, CLA, CD161, 02M, and CIITA polypeptide and antigenic fragments thereof.

By “immunogen encoding polynucleotide” is meant a nucleic acid molecule that encodes an immunogen.

By “immunomodulatory activity” is meant increasing, decreasing, or sustaining an immune response.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the present disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the present disclosure that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the present disclosure. An isolated polypeptide of the present disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In some embodiments, a linker comprises amino acids, nucleic acids, or analogs thereof. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). In some embodiments, the mutation is a missense mutation. In some embodiments, the missense mutation tunes the stability or bioactivity of β2M or components of the peptide loading complex (PLC). In some embodiments, mutations as provided herein are within a peptide binding site, ATP binding site, splice site, promoter, enhancer, or in an untranslated region (UTR).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (2′-e.g., fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
or
(SEQ ID NO: 196)
MDSLLMNRRKFLYQFKNVRWAKGRRETYLC.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2cl, Cas12c/C2c3, Cas12d/CasY, Cas12e/CasX, Cas12g, Cas12h, Cas12i, and Cas12j/Cas4′ (Cas12j/Casphi). Non-limiting examples of Cas enzymes include CasT, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2cl, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-230 and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female. In embodiments the subject is allogeneic to cells administered to the subject. “Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene.

The term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g. physiologically compatible, sterile, physiologic pH, etc.). The terms such as “excipient,” “carrier,” “pharmaceutically acceptable carrier,” “vehicle,” or the like are used interchangeably herein.

The term “pharmaceutical composition” means a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

By “Programmed Cell Death-Ligand 1 (PD-L1) polypeptide” is meant a protein having at least about 85% amino acid sequence identity to NCBI Accession No. NP_054862.1, which is provided below, or a fragment thereof capable of modulating an immune response.

| (SEQ ID NO: 467) | |
|---|---|
| 1 | mrifavfifm tywhllnaft vtvpkdlyvv eygsnmtiec kfpvekqldl aalivyweme |
| 61 | dkniiqfvhg eedlkvqhss yrqrarllkd qlslgnaalq itdvklqdag vyrcmisygg |
| 121 | adykritvkv napynkinqr ilvvdpvtse heltcqaegy pkaeviwtss dhqvlsgktt |
| 181 | ttnskreekl fnvtstlrin tttneifyct frrldpeenh taelvipelp lahppnerth |
| 241 | lvilgaillc lgvaltfifr lrkgrmmdvk kcgiqdtnsk kqsdthleet. |

By “Programmed Cell Death-Ligand 1 (PD-L1) polynucleotide” is meant a nucleic acid molecule encoding an PD-L1 polypeptide, as well as the introns, exons, 3′ untranslated regions, 5′ untranslated regions, and regulatory sequences associated with its expression, or fragments thereof. An exemplary PD-L1 polynucleotide is provided at NCBI Accession No. NM_014143.4, which is provided below.

| (SEQ ID NO: 468) | |
|---|---|
| 1 | agttctgcgc agcttcccga ggctccgcac cagccgcgct tctgtccgcc tgcagggcat |
| 61 | tccagaaaga tgaggatatt tgctgtcttt atattcatga cctactggca tttgctgaac |
| 121 | gcatttactg tcacggttcc caaggaccta tatgtggtag agtatggtag caatatgaca |
| 181 | attgaatgca aattcccagt agaaaaacaa ttagacctgg ctgcactaat tgtctattgg |
| 241 | gaaatggagg ataagaacat tattcaattt gtgcatggag aggaagacct gaaggttcag |
| 301 | catagtagct acagacagag ggcccggctg ttgaaggacc agctctccct gggaaatgct |
| 361 | gcacttcaga tcacagatgt gaaattgcag gatgcagggg tgtaccgctg catgatcagc |
| 421 | tatggtggtg ccgactacaa gcgaattact gtgaaagtca atgccccata caacaaaatc |
| 481 | aaccaaagaa ttttggttgt ggatccagtc acctctgaac atgaactgac atgtcaggct |
| 541 | gagggctacc ccaaggccga agtcatctgg acaagcagtg accatcaagt cctgagtggt |
| 601 | aagaccacca ccaccaattc caagagagag gagaagcttt tcaatgtgac cagcacactg |
| 661 | agaatcaaca caacaactaa tgagattttc tactgcactt ttaggagatt agatcctgag |
| 721 | gaaaaccata cagctgaatt ggtcatccca gaactacctc tggcacatcc tccaaatgaa |
| 781 | aggactcact tggtaattct gggagccatc ttattatgcc ttggtgtagc actgacattc |
| 841 | atcttccgtt taagaaaagg gagaatgatg gatgtgaaaa aatgtggcat ccaagataca |
| 901 | aactcaaaga agcaaagtga tacacatttg gaggagacgt aatccagcat tggaacttct |
| 961 | gatcttcaag cagggattct caacctgtgg tttaggggtt catcggggct gagcgtgaca |
| 1021 | agaggaagga atgggcccgt gggatgcagg caatgtggga cttaaaaggc ccaagcactg |
| 1081 | aaaatggaac ctggcgaaag cagaggagga gaatgaagaa agatggagtc aaacagggag |
| 1141 | cctggaggga gaccttgata ctttcaaatg cctgaggggc tcatcgacgc ctgtgacagg |
| 1201 | gagaaaggat acttctgaac aaggagcctc caagcaaatc atccattgct catcctagga |
| 1261 | agacgggttg agaatcccta atttgagggt cagttcctgc agaagtgccc tttgcctcca |
| 1321 | ctcaatgcct caatttgttt tctgcatgac tgagagtctc agtgttggaa cgggacagta |
| 1381 | tttatgtatg agtttttcct atttattttg agtctgtgag gtcttcttgt catgtgagtg |
| 1441 | tggttgtgaa tgatttcttt tgaagatata ttgtagtaga tgttacaatt ttgtcgccaa |
| 1501 | actaaacttg ctgcttaatg atttgctcac atctagtaaa acatggagta tttgtaaggt |
| 1561 | gcttggtctc ctctataact acaagtatac attggaagca taaagatcaa accgttggtt |
| 1621 | gcataggatg tcacctttat ttaacccatt aatactctgg ttgacctaat cttattctca |
| 1681 | gacctcaagt gtctgtgcag tatctgttcc atttaaatat cagctttaca attatgtggt |
| 1741 | agcctacaca cataatctca tttcatcgct gtaaccaccc tgttgtgata accactatta |
| 1801 | ttttacccat cgtacagctg aggaagcaaa cagattaagt aacttgccca aaccagtaaa |
| 1861 | tagcagacct cagactgcca cccactgtcc ttttataata caatttacag ctatatttta |
| 1921 | ctttaagcaa ttcttttatt caaaaaccat ttattaagtg cccttgcaat atcaatcgct |
| 1981 | gtgccaggca ttgaatctac agatgtgagc aagacaaagt acctgtcctc aaggagctca |
| 2041 | tagtataatg aggagattaa caagaaaatg tattattaca atttagtcca gtgtcatagc |
| 2101 | ataaggatga tgcgagggga aaacccgagc agtgttgcca agaggaggaa ataggccaat |
| 2161 | gtggtctggg acggttggat atacttaaac atcttaataa tcagagtaat tttcatttac |
| 2221 | aaagagaggt cggtacttaa aataaccctg aaaaataaca ctggaattcc ttttctagca |
| 2281 | ttatatttat tcctgatttg cctttgccat ataatctaat gcttgtttat atagtgtctg |
| 2341 | gtattgttta acagttctgt cttttctatt taaatgccac taaattttaa attcatacct |
| 2401 | ttccatgatt caaaattcaa aagatcccat gggagatggt tggaaaatct ccacttcatc |
| 2461 | ctccaagcca ttcaagtttc ctttccagaa gcaactgcta ctgcctttca ttcatatgtt |
| 2521 | cttctaaaga tagtctacat ttggaaatgt atgttaaaag cacgtatttt taaaattttt |
| 2581 | ttcctaaata gtaacacatt gtatgtctgc tgtgtacttt gctattttta tttattttag |
| 2641 | tgtttcttat atagcagatg gaatgaattt gaagttccca gggctgagga tccatgcctt |
| 2701 | ctttgtttct aagttatctt toccatagct tttcattatc tttcatatga tccagtatat |
| 2761 | gttaaatatg tcctacatat acatttagac aaccaccatt tgttaagtat ttgctctagg |
| 2821 | acagagtttg gatttgttta tgtttgctca aaaggagacc catgggctct ccagggtgca |
| 2881 | ctgagtcaat ctagtcctaa aaagcaatct tattattaac tctgtatgac agaatcatgt |
| 2941 | ctggaacttt tttttctgc tttctgtcaa gtataaactt cactttgatg ctgtacttgc |
| 3001 | aaaatcacat tttctttctg gaaattccgg cagtgtacct tgactgctag ctaccctgtg |
| 3061 | ccagaaaagc ctcattcgtt gtgcttgaac ccttgaatgc caccagctgt catcactaca |
| 3121 | cagccctcct aagaggcttc ctggaggttt cgagattcag atgccctggg agatcccaga |
| 3181 | gtttcctttc cctcttggcc atattctggt gtcaatgaca aggagtacct tggctttgcc |
| 3241 | acatgtcaag gctgaagaaa cagtgtctcc aacagagctc cttgtgttat ctgtttgtac |
| 3301 | atgtgcattt gtacagtaat tggtgtgaca gtgttctttg tgtgaattac aggcaagaat |
| 3361 | tgtggctgag caaggcacat agtctactca gtctattcct aagtcctaac tcctccttgt |
| 3421 | ggtgttggat ttgtaaggca ctttatccct tttgtctcat gtttcatcgt aaatggcata |
| 3481 | ggcagagatg atacctaatt ctgcatttga ttgtcacttt ttgtacctgc attaatttaa |
| 3541 | taaaatattc ttatttattt tgttacttgg tacaccagca tgtccatttt cttgtttatt |
| 3601 | ttgtgtttaa taaaatgttc agtttaacat ccca. |

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

By “rBE4 polypeptide” is meant a polypeptide sharing at least 85% amino acid sequence identity to the below amino acid sequence and having cytidine base editor activity.

(SEQ ID NO: 670)
MSSETGPVAVDPTLRRRIEPHEFEVFFDPRELRKETCLLYEINWGG
RHSIWRHTSQNTNKHVEVNFIEKFTTERYFCPNTRCSITWFLSWS
PCGECSRAITEFLSRYPHVTLFIYIARLYHHADPRNRQGLRDLIS
SGVTIQIMTEQESGYCWRNFVNYSPSNEAHWPRYPHLWVRLYVLE
LYCIILGLPPCLNILRRKQPQLTFFTIALQSCHYQRLPPHILWAT
GLKSGGSSGGSSGSETPGTSESATPESSGGSSGGSDKKYSIGLAI
GTNSVGWAVITDEYKVPSKKFKVLGNTDRHSIKKNLIGALLFDSG
ETAEATRLKRTARRRYTRRKNRICYLQEIFSNEMAKVDDSFFHRL
EESFLVEEDKKHERHPIFGNIVDEVAYHEKYPTIYHLRKKLVDST
DKADLRLIYLALAHMIKFRGHFLIEGDLNPDNSDVDKLFIQLVQT
YNQLFEENPINASGVDAKAILSARLSKSRRLENLIAQLPGEKKNG
LFGNLIALSLGLTPNFKSNFDLAEDAKLQLSKDTYDDDLDNLLAQ
IGDQYADLFLAAKNLSDAILLSDILRVNTEITKAPLSASMIKRYD
EHHQDLTLLKALVRQQLPEKYKEIFFDQSKNGYAGYIDGGASQEE
FYKFIKPILEKMDGTEELLVKLNREDLLRKQRTFDNGSIPHQIHL
GELHAILRRQEDFYPFLKDNREKIEKILTFRIPYYVGPLARGNSR
FAWMTRKSEETITPWNFEEVVDKGASAQSFIERMTNFDKNLPNEK
VLPKHSLLYEYFTVYNELTKVKYVTEGMRKPAFLSGEQKKAIVDL
LFKTNRKVTVKQLKEDYFKKIECFDSVEISGVEDRFNASLGTYHD
LLKIIKDKDFLDNEENEDILEDIVLTLTLFEDREMIEERLKTYAH
LFDDKVMKQLKRRRYTGWGRLSRKLINGIRDKQSGKTILDFLKSD
GFANRNFMQLIHDDSLTFKEDIQKAQVSGQGDSLHEHIANLAGSP
AIKKGILQTVKVVDELVKVMGRHKPENIVIEMARENQTTQKGQKN
SRERMKRIEEGIKELGSQILKEHPVENTQLQNEKLYLYYLQNGRD
MYVDQELDINRLSDYDVDHIVPQSFLKDDSIDNKVLTRSDKNRGK
SDNVPSEEVVKKMKNYWRQLLNAKLITQRKFDNLTKAERGGLSEL
DKAGFIKRQLVETRQITKHVAQILDSRMNTKYDENDKLIREVKVI
TLKSKLVSDFRKDFQFYKVREINNYHHAHDAYLNAVVGTALIKKY
PKLESEFVYGDYKVYDVRKMIAKSEQEIGKATAKYFFYSNIMNFF
KTEITLANGEIRKRPLIETNGETGEIVWDKGRDFATVRKVLSMPQ
VNIVKKTEVQTGGFSKESILPKRNSDKLIARKKDWDPKKYGGFDS
PTVAYSVLVVAKVEKGKSKKLKSVKELLGITIMERSSFEKNPIDF
LEAKGYKEVKKDLIIKLPKYSLFELENGRKRMLASAGELQKGNEL
ALPSKYVNFLYLASHYEKLKGSPEDNEQKQLFVEQHKHYLDEIIE
QISEFSKRVILADANLDKVLSAYNKHRDKPIREQAENIIHLFTLT
NLGAPAAFKYFDTTIDRKRYTSTKEVLDATLIHQSITGLYETRID
LSQLGGDSGGSGGSGGSTNLSDIIEKETGKQLVIQESILMLPEEV
EEVIGNKPESDILVHTAYDESTDENVMLLTSDAPEYKPWALVIQD
SNGENKIKMLSGGSGGSGGSTNLSDIIEKETGKQLVIQESILMLP
EEVEEVIGNKPESDILVHTAYDESTDENVMLLTSDAPEYKPWALV
IQDSNGENKIKMLSGGSKRTADGSEFESPKKKRKVE.

By “rBE4 polynucleotide” is meant a polynucleotide encoding a rBE4 polypeptide.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%. In some embodiments, a modified immune cell has a reduction in the level of an immunogenic polypeptide. In embodiments, reduction in the level of an immunogenic polypeptide renders the immunogenic polypeptide undetectable or virtually undetectable. In embodiments, the modified immune cell lacks the immunogenic polypeptide.

By “reference” is meant a standard or control condition. In one embodiment, the reference is a wild-type or healthy cell. In one embodiment, the reference is an unedited cell or an unedited cell that is allogeneic to a host or subject. In other embodiments and without limitation, a reference is an untreated cell that is not subjected to a test condition, or is subjected to placebo or normal saline, medium, buffer, and/or a control vector that does not harbor a polynucleotide of interest.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), or derivatives thereof (e.g. a sequence with at least about 85% sequence identity to a Cas9, such as Nme2Cas9 or spCas9).

The term “single nucleotide polymorphism (SNP)” is a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the present disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. COBALT is used, for example, with the following parameters:
a) alignment parameters: Gap penalties-11,-1 and End-Gap penalties-5,-1,
b) CDD Parameters: Use RPS BLAST on; Blast E-value 0.003; Find Conserved columns and Recompute on, and
c) Query Clustering Parameters: Use query clusters on; Word Size 4; Max cluster distance 0.8; Alphabet Regular.
   EMBOSS Needle is used, for example, with the following parameters:
a) Matrix: BLOSUM62;
b) GAP OPEN: 10;
c) GAP EXTEND: 0.5;
d) OUTPUT FORMAT: pair;
e) END GAP PENALTY: false;
f) END GAP OPEN: 10; and
g) END GAP EXTEND: 0.5.

Nucleic acid molecules useful in the methods of the present disclosure include any nucleic acid molecule that encodes a polypeptide of the present disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the present disclosure include any nucleic acid molecule that encodes a polypeptide of the present disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “split” is meant divided into two or more fragments.

A “split Cas9 protein” or “split Cas9” refers to a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a “reconstituted” Cas9 protein.

By “TAP-associated glycoprotein (Tapasin; TAPBP) polypeptide” is meant a protein having at least about 85% amino acid sequence identity to NCBI Accession No. NP_003181.3, which is provided below, or a fragment thereof capable of modulating an immune response.

```
(SEQ ID NO: 469)
    1 mkslslllav alglatavsa gpaviecwfv
        edasgkglak rpgalllrqg pgeppprpdl
  61 dpelylsvhd pagalqaafr ryprgapaph
        cemsrfvplp asakwasglt paqncprald
121 gawlmvsiss pvlslssllr pqpepqqepv
        litmatvvlt vlthtpaprv rlgqdalldl
181 sfaympptse aasslapgpp pfglewrrqh
        lgkghlllaa tpglngqmpa agegavafaa
241 wdddepwgpw tgngtfwlpt vqpfqegtyl
        atihlpylqg qvtlelavyk ppkvslmpat
301 laraapgeap pellclvshf ypsgglevew
        elrggpggrs qkaegqrwls alrhhsdgsv
361 slsghlqppp vtteqhgary acrihhpslp
        asgrsaevtl evaglsgpsl edsvglflsa
421 flllglfkal gwaavylstc kdskkkae.
```

By “TAP-associated glycoprotein (Tapasin; TAPBP) polynucleotide” is meant a nucleic acid molecule encoding a Tapasin polypeptide, as well as the introns, exons, 3′ untranslated regions, 5′ untranslated regions, and regulatory sequences associated with its expression, or fragments thereof. An exemplary Tapasin polynucleotide is provided at NCBI Accession No. NM_003190.5, which is provided below.

```
(SEQ ID NO: 470)
      1 gaagaggagg cttcatggct gaggaggtcg
          cagcgccatg aagtccctgt ctctgctcct
    61 cgctgtggct ttgggcctgg cgaccgccgt
          ctcagcagga cccgcggtga tcgagtgttg
  121 gttcgtggag gatgcgagcg gaaagggcct
          ggccaagaga cccggtgcac tgctgttgcg
  181 ccagggaccg ggggaaccgc cgccccggcc
          ggacctcgac cctgagctct atctcagtgt
  241 acacgacccc gcgggcgccc tccaggctgc
          cttcaggcgg tatccccggg gcgcccccgc
  301 accacactgc gagatgagcc gcttcgtgcc
          tctccccgcc tctgcgaaat gggccagcgg
  361 cctgaccccc gcgcagaact gcccgcgggc
          cctggatggg gcttggctga tggtcagcat
  421 atccagccca gtcctcagcc tctccagcct
          cttgcgacca cagccagagc ctcagcagga
  481 gcctgttctc atcaccatgg caacagtggt
          actgactgtc ctcacccaca cccctgcccc
  541 tcgagtgaga ctgggacaag atgctctgct
          ggacttgagc tttgcctaca tgccccccac
  601 ctccgaggcc gcctcatctc tggctccggg
          tccccctccc tttgggctag agtggcgacg
  661 ccagcacctg ggtaagggac atctgctcct
          ggctgcaact cctgggctga atggccagat
  721 gccagcagcc caagaagggg ccgtggcatt
          tgctgcttgg gatgatgatg agccatgggg
  781 cccatggacc ggaaatggga ccttctggct
          gcctacagtt caaccctttc aggagggcac
  841 ctatctggcc accatacacc tgccatacct
          gcaaggacag gtcaccctgg agcttgctgt
  901 gtacaaaccc cccaaagtgt ccctgatgcc
          agcaaccctt gcacgggccg ccccagggga
  961 ggcacccccg gaattgctct gccttgtgtc
          ccacttctac ccttctgggg gcctggaggt
1021 ggagtgggaa ctccggggtg gcccaggggg
          ccgctctcag aaggccgagg ggcagaggtg
1081 gctctcggcc ctgcgccacc attccgatgg
          ctctgtcagc ctctctgggc acttgcagcc
1141 gcccccagtc accactgagc agcatggggc
          acgctatgcc tgtcgaattc accatcccag
1201 cctgcctgcc tcggggcgca gcgctgaggt
          caccctggag gtagcaggtc tttcagggcc
1261 ctcccttgag gacagcgtag gccttttcct
          gtctgccttt cttctgcttg ggctcttcaa
1321 ggcactgggc tgggctgctg tctacctgtc
          cacctgcaag gattcaaaga agaaagcaga
1381 gtgagggcac tcactgccat cctgtggaag
          ccaccatcat ctctggccca agcttctgta
1441 gtagctccct aaaataatac cctatcatct
          gctcctaatc cctccaatct ctctccactg
1501 agtggctgga atgctttttt ttttttcttt
          cacttatata agggataatt tttctttttt
1561 tttttttttt gagacggagt ctcactcttc
          cgcccaggct gcagtgcagt ggcatgatct
1621 tggcttactg caacctccgc ctcctgggtt
          caagcaattc tgtggcttca gcctccggag
1681 tagctgggat tacaggcaca tgccaccaca
          cccagtgaat ttttgtattt ttagtagaga
1741 cggggtttca ccatgttggc caggctggtc
          ttgaattcct gacctcaggt gatctgccca
1801 cctcagcctc ccaaagtgct gggattacag
          gcgtgagcca ccacaccagg cccgagaaat
1861 gcttttttaa aaaacacaca tcttatggca
          ttcaccttct tggagctcta ggacagtggt
1921 tctcaaaatt tttttctctc aggacctctt
          aaaaatcatc aaggacccca aaaagctttt
1981 gggtatgtgg gttatagcta tcaatattta
          tggtactaga acttaaaagt gagaaaaatt
2041 taaaacacga gaatacatag gcacacattc
          tattcatcgt gggaaccatg gtgtcaatac
2101 atatcatgta gcttctgaaa aactccactg
          tacacttata gaatgaagaa ggcaaaaaac
2161 tttttttttt ttttttttga gacggagtct
          cgctctgtcg cccaggctgg agtgcagtgg
2221 cgcgatctcg gctcactgca agctccgcct
          ctcgggttca cgccattctc ctgcctcagc
2281 ctcccaagta gctcggacta caggcgtcct
          ccaccatgcc tggctaatat tttgtatttt
2341 ttagtagaga cggggtttca ccgtgttagc
          caggatggtc tcgatctcct aacctggtga
2401 tccgcccgcc tcggcctccc aaagtattgg
          gattacccgc gtgagccacc gcgcccggct
2461 gcaaataatc tttctttttt tctgagacag
          agtctcgctc tgttgcccag gctggagtgc
2521 agtggcacga tctcggctca cggcacgctc
          cgcctcccgg gttcacgcca ttctcctgcc
2581 tcagcttccc gagtagctgg gactacaggg
          gcccgccacc acgcccggct aactttttgt
2641 gtttttagta gagacggggt ttcaccgtgt
          tagccaggat ggtctcgatc tcctgacctt
2701 gtgatctgcc cgcctcggcc tcccaaagtg
          ctgggattac aggcgtgagc caccgcgccc
2761 ggcggcgaaa cacgatattg tactaacatc
          ttaattttgt tataaaatct cacaaacccc
2821 ctgacatagt ctcagagatc tgtagggccg
          aggttacatt tggagaaccc gtactctagg
2881 gccaaatcca ttcttcttgc cctggctcac
          ttgtcccccc caccgccccg cgctggagcc
2941 actgcctagt tcttcagccc tagatggtgc
          tcgccagacc tcctctcaat gctcatcaca
3001 cacagggcta ttcctttcct ccaatgaacc
          aaacgcctcc cgcccacctc caggtcccag
3061 tcctctgttc cctttgcctg gtccaccctt
          gccctccctg ggtcgcagac gaggtcggcc
3121 tcgtcattcc ccgcagaccg ccgcgcgtcc
          ctcttgtgcg gttcaccaca gttgtattta
3181 agtgatcgtg tgagtcgtcg ttaaatgcct
          gtctccccgc ggatcatggg ctcctcgagg
3241 acagggactg gcctgtctgt ccactgctgt
          aaccccgcgc cggcataggg acctaaggcc
3301 cactggaggg cgctcatcaa gtagctgctg
          gatgttgacg aaggaagcgg cggcgcagct
3361 cagggatctc cgagtcagga cggtcggcca
          gacccacggg gtaacgggtc taatcgtgta
3421 ggaataaagc tgtattccag tgcttccaaa.
The tapasin gene corresponds to
ENSG00000231925, ENSG00000236490,
ENSG00000206281 ENSG00000206208,
and ENSG00000112493.
```

By “TAP binding protein-like (TAPBPL) polypeptide” is meant a protein having at least about 85% amino acid sequence identity to GenBank Accession No. AAH15017.1, which is provided below, or a fragment thereof having immunomodulatory activity.

>AAH15017.1 TAP binding protein-like
[Homo sapiens]
(SEQ ID NO: 1118)
MGTQEGWCLLLCLALSGAAETKPHPAEGQWRAVDVVLDCFLVKDG
AHRGALASSEDRARASLVLKQVPVLDDGSLEDFTDFQGGTLAQDD
PPIIFEASVDLVQIPQAEALLHADCSGKEVTCEISRYFLQMTETT
VKTAAWFMANVQVSGGGPSISLVMKTPRVAKNEVLWHPTLNLPLS
PQGTVRTAVEFQVMTQTQSLSFLLGSSASLDCGFSMAPGLDLISV
EWRLQHKGRGQLVYSWTAGQGQAVRKGATLEPAQLGMARDASLTL
PGLTIQDEGTYICQITTSLYRAQQIIQLNIQASPKVRLSLANEAL
LPTLICDIAGYYPLDVVVTWTREELGGSPAQVSGASFSSLRQSVA
GTYSISSSLTAEPGSAGATYTCQVTHISLEEPLGASTQVVPPERR
TALGVIFASSLFLLALMFLGLQRRQAPTGLGLLQAERWETTSCAD
TQSSHLHEDRTARVSQPS.

By “TAP binding protein-like (TAPBPL) polynucleotide” is meant a nucleic acid molecule encoding an TAPBPL polypeptide, as well as the introns, exons, 3′ untranslated regions, 5′ untranslated regions, and regulatory sequences associated with its expression, or fragments thereof. In embodiments, a TAPBPL polynucleotide is the genomic sequence, cDNA, mRNA, or gene associated with and/or required for TAPBPL expression. An exemplary TAPBPL polynucleotide sequence from Homo sapiens is provided at GenBank Accession No. BC015017.2 which is provided below, and at NCBI Ref. Seq. Accession No. NC_000012.12:6451649-6472006, which is provided in the Sequence Listing as SEQ ID NO: 1120.

>BC015017.2 Homo sapiens TAP binding protein-
like, mRNA (cDNA clone MGC: 8836
IMAGE: 3916213), complete cds
(SEQ ID NO: 1119)
GTGAAAGTGAAAGAAAAGTCGGCAGCAGAGGGAACAGGGAAGAAA
CCTAAAGGCTGCAGGCTGCCAGGTGTGCTTGGAGAGCCCCCTTCT
TCCGCCGGGCCTCGCAAGCAGCGTAGGACTGTGGAGAAGGGCGGT
GGGCAAGGAGGGAACTCGAGAGCAGCCTCCATGGGCACACAGGAG
GGCTGGTGCCTGCTGCTCTGCCTGGCTCTATCTGGAGCAGCAGAA
ACCAAGCCCCACCCAGCAGAGGGGCAGTGGCGGGCAGTGGACGTG
GTCCTAGACTGTTTCCTGGTGAAGGACGGTGCGCACCGTGGAGCT
CTCGCCAGCAGTGAGGACAGGGCAAGGGCCTCCCTTGTGCTGAAG
CAGGTGCCAGTGCTGGACGATGGCTCCCTGGAGGACTTCACCGAT
TTCCAAGGGGGCACACTGGCCCAAGATGACCCACCTATTATCTTT
GAGGCCTCAGTGGACCTGGTCCAGATTCCCCAGGCCGAGGCCTTG
CTCCATGCTGACTGCAGTGGGAAGGAGGTGACCTGTGAGATCTCC
CGCTACTTTCTCCAGATGACAGAGACCACTGTTAAGACAGCAGCT
TGGTTCATGGCCAACGTGCAGGTCTCTGGAGGGGGACCTAGCATC
TCCTTGGTGATGAAGACTCCCAGGGTCGCCAAGAATGAGGTGCTC
TGGCACCCAACGCTGAACTTGCCACTGAGCCCCCAGGGGACTGTG
CGAACTGCAGTGGAGTTCCAGGTGATGACACAGACCCAATCCCTG
AGCTTCCTGCTGGGGTCCTCAGCCTCCTTGGACTGTGGCTTCTCC
ATGGCACCGGGCTTGGACCTCATCAGTGTGGAGTGGCGACTGCAG
CACAAGGGCAGGGGTCAGTTGGTGTACAGCTGGACCGCAGGGCAG
GGGCAGGCTGTGCGGAAGGGCGCTACCCTGGAGCCTGCACAACTG
GGCATGGCCAGGGATGCCTCCCTCACCCTGCCCGGCCTCACTATA
CAGGACGAGGGGACCTACATTTGCCAGATCACCACCTCTCTGTAC
CGAGCTCAGCAGATCATCCAGCTCAACATCCAAGCTTCCCCTAAA
GTACGACTGAGCTTGGCAAACGAAGCTCTGCTGCCCACCCTCATC
TGCGACATTGCTGGCTATTACCCTCTGGATGTGGTGGTGACGTGG
ACCCGAGAGGAGCTGGGTGGATCCCCAGCCCAAGTCTCTGGTGCC
TCCTTCTCCAGCCTCAGGCAAAGCGTGGCAGGCACCTACAGCATC
TCCTCCTCTCTCACCGCAGAACCTGGCTCTGCAGGTGCCACTTAC
ACCTGCCAGGTCACACACATCTCTCTGGAGGAGCCCCTTGGGGCC
AGCACCCAGGTTGTCCCACCAGAGCGGAGAACAGCCTTGGGAGTC
ATCTTTGCCAGCAGTCTCTTCCTTCTTGCACTGATGTTCCTGGGG
CTTCAGAGACGGCAAGCACCTACAGGACTTGGGCTGCTTCAGGCT
GAACGCTGGGAGACCACTTCCTGTGCTGACACACAGAGCTCCCAT
CTCCATGAAGACCGCACAGCGCGTGTAAGCCAGCCCAGCTGACCT
AAAGCGACATGAGACTACTAGAAAGAAACGACACCCTTCCCCAAG
CCCCCACAGCTACTCCAACCCAAACAACAACCAAGCCAGTTTAAT
GGTAGGAATTTGTATTTTTTGCCTTTGTTCAGAATACATGACATT
GGTAAATAAAAAAAAAAAAAAAAAAAAAA.

The term “target site” refers to a sequence within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

By “T Cell Receptor Alpha Constant (TRAC) polypeptide” is meant a protein having at least about 85% amino acid sequence identity to NCBI Accession No. P01848.2, or a fragment thereof having immunomodulatory activity. An exemplary amino acid sequence is provided below.

(SEQ ID NO: 471)
>sp|P01848.2|TRAC_HUMAN RecName:
Full = T cell receptor alpha constant
IQNPDPAVYQLRDSKSSDKSVCLFTDFDSQTNVSQSKDSDVYITD
KTVLDMRSMDFKSNSAVAWSNKSDFACANAFNNSIIPEDTFFPSP
ESSCDVKLVEKSFETDTNLNFQNLSVIGFRILLLKVAGFNLLMTL
RLWSS.

By “T Cell Receptor Alpha Constant (TRAC) polynucleotide” is meant a nucleic acid molecule encoding a TRAC polypeptide, as well as the introns, exons, 3′ untranslated regions, 5′ untranslated regions, and regulatory sequences associated with its expression, or fragments thereof. An exemplary TRAC polynucleotide is provided at Gene ENSG00000277734.8, which is provided below.

>UCSC human genome database, Gene
ENSG00000277734.8 Human T-cell receptor
alpha chain (TCR-alpha)
(SEQ ID NO: 472)
catgctaatcctccggcaaacctctgtttcctcctcaaaaggcag
gaggtcggaaagaataaacaatgagagtcacattaaaaacacaaa
atcctacggaaatactgaagaatgagtctcagcactaaggaaaag
cctccagcagctcctgctttctgagggtgaaggatagacgctgtg
gctctgcatgactcactagcactctatcacggccatattctggca
gggtcagtggctccaactaacatttgtttggtactttacagttta
ttaaatagatgtttatatggagaagctctcatttctttctcagaa
gagcctggctaggaaggtggatgaggcaccatattcattttgcag
gtgaaattcctgagatgtaaggagctgctgtgacttgctcaaggc
cttatatcgagtaaacggtagtgctggggcttagacgcaggtgtt
ctgatttatagttcaaaacctctatcaatgagagagcaatctcct
ggtaatgtgatagatttcccaacttaatgccaacataccataaac
ctcccattctgctaatgcccagcctaagttggggagaccactcca
gattccaagatgtacagtttgctttgctgggcctttttcccatgc
ctgcctttactctgccagagttatattgctggggttttgaagaag
atcctattaaataaaagaataagcagtattattaagtagccctgc
atttcaggtttccttgagtggcaggccaggcctggccgtgaacgt
tcactgaaatcatggcctcttggccaagattgatagcttgtgcct
gtccctgagtcccagtccatcacgagcagctggtttctaagatgc
tatttcccgtataaagcatgagaccgtgacttgccagccccacag
agccccgcccttgtccatcactggcatctggactccagcctgggt
tggggcaaagagggaaatgagatcatgtcctaaccctgatcctct
tgtcccacagATATCCAGAACCCTGACCCTGCCGTGTACCAGCTG
AGAGACTCTAAATCCAGTGACAAGTCTGTCTGCCTATTCACCGAT
TTTGATTCTCAAACAAATGTGTCACAAAGTAAGGATTCTGATGTG
TATATCACAGACAAAACTGTGCTAGACATGAGGTCTATGGACTTC
AAGAGCAACAGTGCTGTGGCCTGGAGCAACAAATCTGACTTTGCA
TGTGCAAACGCCTTCAACAACAGCATTATTCCAGAAGACACCTTC
TTCCCCAGCCCAGgtaagggcagctttggtgccttcgcaggctgt
ttccttgcttcaggaatggccaggttctgcccagagctctggtca
atgatgtctaaaactcctctgattggtggtctcggccttatccat
tgccaccaaaaccctctttttactaagaaacagtgagccttgttc
tggcagtccagagaatgacacgggaaaaaagcagatgaagagaag
gtggcaggagagggcacgtggcccagcctcagtctctccaactga
gttcctgcctgcctgcctttgctcagactgtttgccccttactgc
tcttctaggcctcattctaagccccttctccaagttgcctctcct
tatttctccctgtctgccaaaaaatctttcccagctcactaagtc
agtctcacgcagtcactcattaacccaccaatcactgattgtgcc
ggcacatgaatgcaccaggtgttgaagtggaggaattaaaaagtc
agatgaggggtgtgcccagaggaagcaccattctagttgggggag
cccatctgtcagctgggaaaagtccaaataacttcagattggaat
gtgttttaactcagggttgagaaaacagctaccttcaggacaaaa
gtcagggaagggctctctgaagaaatgctacttgaagataccagc
cctaccaagggcagggagaggaccctatagaggcctgggacagga
gctcaatgagaaaggagaagagcagcaggcatgagttgaatgaag
gaggcagggccgggtcacagggccttctaggccatgagagggtag
acagtattctaaggacgccagaaagctgttgatcggcttcaagca
ggggagggacacctaatttgcttttcttttttttttttttttttt
tttttttttttgagatggagttttgctcttgttgcccaggctgga
gtgcaatggtgcatcttggctcactgcaacctccgcctcccaggt
tcaagtgattctcctgcctcagcctcccgagtagctgagattaca
ggcacccgccaccatgcctggctaattttttgtatttttagtaga
gacagggtttcactatgttggccaggctggtctcgaactcctgac
ctcaggtgatccacccgcttcagcctcccaaagtgctgggattac
aggcgtgagccaccacacccggcctgcttttcttaaagatcaatc
tgagtgctgtacggagagtgggttgtaagccaagagtagaagcag
aaagggagcagttgcagcagagagatgatggaggcctgggcaggg
tggtggcagggaggtaaccaacaccattcaggtttcaaaggtaga
accatgcagggatgagaaagcaaagaggggatcaaggaaggcagc
tggattttggcctgagcagctgagtcaatgatagtgccgtttact
aagaagaaaccaaggaaaaaatttggggtgcagggatcaaaactt
tttggaacatatgaaagtacgtgtttatactctttatggcccttg
tcactatgtatgcctcgctgcctccattggactctagaatgaagc
caggcaagagcagggtctatgtgtgatggcacatgtggccagggt
catgcaacatgtactttgtacaaacagtgtatattgagtaaatag
aaatggtgtccaggagccgaggtatcggtcctgccagggccaggg
gctctccctagcaggtgctcatatgctgtaagttccctccagatc
tctccacaaggaggcatggaaaggctgtagttgttcacctgccca
agaactaggaggtctggggtgggagagtcagcctgctctggatgc
tgaaagaatgtctgtttttccttttagAAAGTTCCTGTGATGTCA
AGCTGGTCGAGAAAAGCTTTGAAACAGgtaagacaggggtctagc
ctgggtttgcacaggattgcggaagtgatgaacccgcaataaccc
tgcctggatgagggagtgggaagaaattagtagatgtgggaatga
atgatgaggaatggaaacagcggttcaagacctgcccagagctgg
gtggggtctctcctgaatccctctcaccatctctgactttccatt
ctaagcactttgaggatgagtttctagcttcaatagaccaaggac
tctctcctaggcctctgtattcctttcaacagctccactgtcaag
agagccagagagagcttctgggtggcccagctgtgaaatttctga
gtcccttagggatagccctaaacgaaccagatcatcctgaggaca
gccaagaggttttgccttctttcaagacaagcaacagtactcaca
taggctgtgggcaatggtcctgtctctcaagaatcccctgccact
cctcacacccaccctgggcccatattcatttccatttgagttgtt
cttattgagtcatccttcctgtggtagcggaactcactaaggggc
ccatctggacccgaggtattgtgatgataaattctgagcacctac
cccatccccagaagggctcagaaataaaataagagccaagtctag
tcggtgtttcctgtcttgaaacacaatactgttggccctggaaga
atgcacagaatctgtttgtaaggggatatgcacagaagctgcaag
ggacaggaggtgcaggagctgcaggcctcccccacccagcctgct
ctgccttggggaaaaccgtgggtgtgtcctgcaggccatgcaggc
ctgggacatgcaagcccataaccgctgtggcctcttggttttaca
gATACGAACCTAAACTTTCAAAACCTGTCAGTGATTGGGTTCCGA
ATCCTCCTCCTGAAAGTGGCCGGGTTTAATCTGCTCATGACGCTG
CGGCTGTGGTCCAGCTGAGgtgaggggccttgaagctgggagtgg
ggtttagggacgcgggtctctgggtgcatcctaagctctgagagc
aaacctccctgcagggtcttgcttttaagtccaaagcctgagccc
accaaactctcctacttcttcctgttacaaattcctcttgtgcaa
taataatggcctgaaacgctgtaaaatatcctcatttcagccgcc
tcagttgcacttctcccctatgaggtaggaagaacagttgtttag
aaacgaagaaactgaggccccacagctaatgagtggaggaagaga
gacacttgtgtacaccacatgccttgtgttgtacttctctcaccg
tgtaacctcctcatgtcctctctccccagtacggctctcttagct
cagtagaaagaagacattacactcatattacaccccaatcctggc
tagagtctccgcaccctcctcccccagggtccccagtcgtcttgc
tgacaactgcatcctgttccatcaccatcaaaaaaaaactccagg
ctgggtgcgggggctcacacctgtaatcccagcactttgggaggc
agaggcaggaggagcacaggagctggagaccagcctgggcaacac
agggagaccccgcctctacaaaaagtgaaaaaattaaccaggtgt
ggtgctgcacacctgtagtcccagctacttaagaggctgagatgg
gaggatcgcttgagccctggaatgttgaggctacaatgagctgtg
attgcgtcactgcactccagcctggaagacaaagcaagatcctgt
ctcaaataataaaaaaaataagaactccagggtacatttgctcct
agaactctaccacatagccccaaacagagccatcaccatcacatc
cctaacagtcctgggtcttcctcagtgtccagcctgacttctgtt
cttcctcattccagATCTGCAAGATTGTAAGACAGCCTGTGCTCC
CTCGCTCCTTCCTCTGCATTGCCCCTCTTCTCCCTCTCCAAACAG
AGGGAACTCTCCTACCCCCAAGGAGGTGAAAGCTGCTACCACCTC
TGTGCCCCCCCGGCAATGCCACCAACTGGATCCTACCCGAATTTA
TGATTAAGATTGCTGAAGAGCTGCCAAACACTGCTGCCACCCCCT
CTGTTCCCTTATTGCTGCTTGTCACTGCCTGACATTCACGGCAGA
GGCAAGGCTGCTGCAGCCTCCCCTGGCTGTGCACATTCCCTCCTG
CTCCCCAGAGACTGCCTCCGCCATCCCACAGATGATGGATCTTCA
GTGGGTTCTCTTGGGCTCTAGGTCCTGCAGAATGTTGTGAGGGGT
TTATTTTTTTTTAATAGTGTTCATAAAGAAATACATAGTATTCTT
CTTCTCAAGACGTGGGGGGAAATTATCTCATTATCGAGGCCCTGC
TATGCTGTGTATCTGGGCGTGTTGTATGTCCTGCTGCCGATGCCT
TCATTAAAATGATTTGGAAGAGCAGA.

Nucleotides in lower case above are untranslated regions or introns, and nucleotides in upper cases are exons.

>X02592.1 Human mRNA for T-cell receptor
alpha chain (TCR-alpha)
(SEQ ID NO: 473)
TTTTGAAACCCTTCAAAGGCAGAGACTTGTCCAGCCTAACCTGCC
TGCTGCTCCTAGCTCCTGAGGCTCAGGGCCCTTGGCTTCTGTCCG
CTCTGCTCAGGGCCCTCCAGCGTGGCCACTGCTCAGCCATGCTCC
TGCTGCTCGTCCCAGTGCTCGAGGTGATTTTTACCCTGGGAGGAA
CCAGAGCCCAGTCGGTGACCCAGCTTGGCAGCCACGTCTCTGTCT
CTGAAGGAGCCCTGGTTCTGCTGAGGTGCAACTACTCATCGTCTG
TTCCACCATATCTCTTCTGGTATGTGCAATACCCCAACCAAGGAC
TCCAGCTTCTCCTGAAGTACACATCAGCGGCCACCCTGGTTAAAG
GCATCAACGGTTTTGAGGCTGAATTTAAGAAGAGTGAAACCTCCT
TCCACCTGACGAAACCCTCAGCCCATATGAGCGACGCGGCTGAGT
ACTTCTGTGCTGTGAGTGATCTCGAACCGAACAGCAGTGCTTCCA
AGATAATCTTTGGATCAGGGACCAGACTCAGCATCCGGCCAAATA
TCCAGAACCCTGACCCTGCCGTGTACCAGCTGAGAGACTCTAAAT
CCAGTGACAAGTCTGTCTGCCTATTCACCGATTTTGATTCTCAAA
CAAATGTGTCACAAAGTAAGGATTCTGATGTGTATATCACAGACA
AAACTGTGCTAGACATGAGGTCTATGGACTTCAAGAGCAACAGTG
CTGTGGCCTGGAGCAACAAATCTGACTTTGCATGTGCAAACGCCT
TCAACAACAGCATTATTCCAGAAGACACCTTCTTCCCCAGCCCAG
AAAGTTCCTGTGATGTCAAGCTGGTCGAGAAAAGCTTTGAAACAG
ATACGAACCTAAACTTTCAAAACCTGTCAGTGATTGGGTTCCGAA
TCCTCCTCCTGAAAGTGGCCGGGTTTAATCTGCTCATGACGCTGC
GGCTGTGGTCCAGCTGAGATCTGCAAGATTGTAAGACAGCCTGTG
CTCCCTCGCTCCTTCCTCTGCATTGCCCCTCTTCTCCCTCTCCAA
ACAGAGGGAACTCTCCTACCCCCAAGGAGGTGAAAGCTGCTACCA
CCTCTGTGCCCCCCCGGTAATGCCACCAACTGGATCCTACCCGAA
TTTATGATTAAGATTGCTGAAGAGCTGCCAAACACTGCTGCCACC
CCCTCTGTTCCCTTATTGCTGCTTGTCACTGCCTGACATTCACGG
CAGAGGCAAGGCTGCTGCAGCCTCCCCTGGCTGTGCACATTCCCT
CCTGCTCCCCAGAGACTGCCTCCGCCATCCCACAGATGATGGATC
TTCAGTGGGTTCTCTTGGGCTCTAGGTCCTGGAGAATGTTGTGAG
GGGTTTATTTTTTTTTAATAGTGTTCATAAAGAAATACATAGTAT
TCTTCTTCTCAAGACGTGGGGGGAAATTATCTCATTATCGAGGCC
CTGCTATGCTGTGTGTCTGGGCGTGTTGTATGTCCTGCTGCCGAT
GCCTTCATTAAAATGATTTGGAA.

By “Transporter associated with antigen processing I (TAP1) polypeptide” is meant a protein having at least about 85% amino acid sequence identity to NCBI Accession No. NP_000584.3, which is provided below, or a fragment thereof capable of modulating an immune response.

```
(SEQ ID NO: 474)
    1 massrcpapr gcrclpgasl awlgtvllll
        adwvllrtal prifsllvpt alpllrvwav
  61 glsrwavlwl gacgvlratv gsksenagaq
        gwlaalkpla aalglalpgl alfreliswg
121 apgsadstrl lhwgshptaf vvsyaaalpa
        aalwhklgsl wvpgggggsg npvrrllgcl
181 gsetrrlslf lvlvvlsslg emaipfftgr
        ltdwilqdgs adtftrnltl msiltiasav
241 lefvgdgiyn ntmghvhshl qgevfgavlr
        qeteffqqnq tgnimsrvte dtstlsdsls
301 enlslflwyl vrglcllgim lwgsvsltmv
        tlitlpllfl lpkkvgkwyq llevqvresl
361 akssqvaiea lsamptvrsf aneegeaqkf
        reklqeiktl nqkeavayav nswttsisgm
421 llkvgilyig gqlvtsgavs sgnlvtfvly
        qmqftqavev llsiyprvqk avgssekife
481 yldrtprcpp sglltplhle glvqfqdvsf
        aypnrpdvlv lqgltftlrp gevtalvgpn
541 gsgkstvaal lqnlyqptgg qllldgkplp
        qyehrylhrq vaavgqepqv fgrslqenia
601 ygltqkptme eitaaavksg ahsfisglpq
        gydtevdeag sqlsggqrqa valaralirk
661 pcvlilddat saldansqlq veqllyespe
        rysrsvllit qhlslvegad hilfleggai
721 reggthqqlm ekkgcywamv qapadape.
```

By “Transporter associated with antigen processing I (TAP1) polynucleotide” is meant a nucleic acid molecule encoding a TAP1 polypeptide, as well as the introns, exons, 3′ untranslated regions, 5′ untranslated regions, and regulatory sequences associated with its expression, or fragments thereof. An exemplary TAP1 polynucleotide is provided at NCBI Accession No. NM_000593.6, which is provided below.

```
(SEQ ID NO: 475)
      1 gcttcaggcc ccaccggggc gcggagagtc
          ccaggcccgg ccgggaccgg gacggcgtcc
    61 gagtgccaat ggctagctct aggtgtcccg
          ctccccgcgg gtgccgctgc ctccccggag
  121 cttctctcgc atggctgggg acagtactgc
          tacttctcgc cgactgggtg ctgctccgga
  181 ccgcgctgcc ccgcatattc tccctgctgg
          tgcccaccgc gctgccactg ctccgggtct
  241 gggcggtggg cctgagccgc tgggccgtgc
          tctggctggg ggcctgcggg gtcctcaggg
  301 caacggttgg ctccaagagc gaaaacgcag
          gtgcccaggg ctggctggct gctttgaagc
  361 cattagctgc ggcactgggc ttggccctgc
          cgggacttgc cttgttccga gagctgatct
  421 catggggagc ccccgggtcc gcggatagca
          ccaggctact gcactgggga agtcacccta
  481 ccgccttcgt tgtcagttat gcagcggcac
          tgcccgcagc agccctgtgg cacaaactcg
  541 ggagcctctg ggtgcccggc ggtcagggcg
          gctctggaaa ccctgtgcgt cggcttctag
  601 gctgcctggg ctcggagacg cgccgcctct
          cgctgttcct ggtcctggtg gtcctctcct
  661 ctcttgggga gatggccatt ccattcttta
          cgggccgcct cactgactgg attctacaag
  721 atggctcagc cgataccttc actcgaaact
          taactctcat gtccattctc accatagcca
  781 gtgcagtgct ggagttcgtg ggtgacggga
          tctataacaa caccatgggc cacgtgcaca
  841 gccacttgca gggagaggtg tttggggctg
          tcctgcgcca ggagacggag tttttccaac
  901 agaaccagac aggtaacatc atgtctcggg
          taacagagga cacgtccacc ctgagtgatt
  961 ctctgagtga gaatctgagc ttatttctgt
          ggtacctggt gcgaggccta tgtctcttgg
1021 ggatcatgct ctggggatca gtgtccctca
          ccatggtcac cctgatcacc ctgcctctgc
1081 ttttccttct gcccaagaag gtgggaaaat
          ggtaccagtt gctggaagtg caggtgcggg
1141 aatctctggc aaagtccagc caggtggcca
          ttgaggctct gtcggccatg cctacagttc
1201 gaagctttgc caacgaggag ggcgaagccc
          agaagtttag ggaaaagctg caagaaataa
1261 agacactcaa ccagaaggag gctgtggcct
          atgcagtcaa ctcctggacc actagtattt
1321 caggtatgct gctgaaagtg ggaatcctct
          acattggtgg gcagctggtg accagtgggg
1381 ctgtaagcag tgggaacctt gtcacatttg
          ttctctacca gatgcagttc acccaggctg
1441 tggaggtact gctctccatc taccccagag
          tacagaaggc tgtgggctcc tcagagaaaa
1501 tatttgagta cctggaccgc acccctcgct
          gcccacccag tggtctgttg actcccttac
1561 acttggaggg ccttgtccag ttccaagatg
          tctcctttgc ctacccaaac cgcccagatg
1621 tcttagtgct acaggggctg acattcaccc
          tacgccctgg cgaggtgacg gcgctggtgg
1681 gacccaatgg gtctgggaag agcacagtgg
          ctgccctgct gcagaatctg taccagccca
1741 ccgggggaca gctgctgttg gatgggaagc
          cccttcccca atatgagcac cgctacctgc
1801 acaggcaggt ggctgcagtg ggacaagagc
          cacaggtatt tggaagaagt cttcaagaaa
1861 atattgccta tggcctgacc cagaagccaa
          ctatggagga aatcacagct gctgcagtaa
1921 agtctggggc ccatagtttc atctctggac
          tccctcaggg ctatgacaca gaggtagacg
1981 aggctgggag ccagctgtca gggggtcagc
          gacaggcagt ggcgttggcc cgagcattga
2041 tccggaaacc gtgtgtactt atcctggatg
          atgccaccag tgccctggat gcaaacagcc
2101 agttacaggt ggagcagctc ctgtacgaaa
          gccctgagcg gtactcccgc tcagtgcttc
2161 tcatcaccca gcacctcagc ctggtggagc
          aggctgacca catcctcttt ctggaaggag
2221 gcgctatccg ggagggggga acccaccagc
          agctcatgga gaaaaagggg tgctactggg
2281 ccatggtgca ggctcctgca gatgctccag
          aatgaaagcc ttctcagacc tgcgcactcc
2341 atctccctcc cttttcttct ctctgtggtg
          gagaaccaca gctgcagagt aggcagctgc
2401 ctccaggatg agttacttga aatttgcctt
          gagtgtgtta cctcctttcc aagctcctcg
2461 tgataatgca gacttcctgg agtacaaaca
          caggatttgt aattccttac tgtaacggag
2521 tttagagcca gggctgatgc tttggtgtgg
          ccagcactct gaaactgaga aatgttcaga
2581 atgtacggaa agatgatcag ctattttcaa
          cataactgaa ggcatatgct ggcccataaa
2641 caccctgtag gttcttgata tttataataa
          aattggtgtt ttgta.
The TAP1 gene corresponds to Ensembl:
ENSG00000168394.
```

By “Transporter associated with antigen processing II (TAP2) polypeptide” is meant a protein having at least about 85% amino acid sequence identity to NCBI Accession No. NP_000535.3, which is provided below, or a fragment thereof capable of modulating an immune response.

(SEQ ID NO: 476)

| | |
|---|---|
| 1 | mrlpdlrpwt slllvdaall wllqgplgtl |
| | lpqglpglwl egtlrlgglw gllklrgllg |
| 61 | fvgtlllplc latpltvslr alvagasrap |
| | parvasapws wllvgygaag lswslwavls |
| 121 | ppgagekeqd qvnnkvlmwr llklsrpdlp |
| | llvaaffflv lavlgetlip hysgrvidil |
| 181 | ggdfdphafa saiffmclfs fgsslsager |
| | ggcftytmsr inlrireqlf ssllrqdlgf |
| 241 | fqetktgeln srlssdttlm snwlplnanv |
| | llrslvkvvg lygfmlsisp rltllsllhm |
| 301 | pftiaaekvy ntrhqevlre iqdavaragq |
| | vvreavgglq tvrsfgaeeh evcrykeale |
| 361 | qcrqlywrrd leralyllvr rvlhlgvqml |
| | mlscglqqmq dgeltqgsll sfmiyqesvg |
| 421 | syvqtlvyiy gdmlsnvgaa ekvfsymdrq |
| | pnlpspgtla pttlqgvvkf qdvsfaypnr |
| 481 | pdrpvlkglt ftlrpgevta lvgpngsgks |
| | tvaallqnly qptggqvlld ekpisqyehc |
| 541 | ylhsqvvsvg qepvlfsgsv rnniayglqs |
| | ceddkvmaaa qaahaddfiq emehgiytdv |
| 601 | gekgsqlaag qkqrlaiara lvrdprvlil |
| | deatsaldvq ceqalqdwns rgdrtvlvia |
| 661 | hrlqavqrah qilvlqegkl qklaqlqegq |
| | dlysrlvqqr lmd. |

By “Transporter associated with antigen processing II (TAP2) polynucleotide” is meant a nucleic acid molecule encoding a TAP2 polypeptide, as well as the introns, exons, 3′ untranslated regions, 5′ untranslated regions, and regulatory sequences associated with its expression, or fragments thereof. An exemplary TAP2 polynucleotide is provided at NCBI Accession No. NM_000544.3, which is provided below.

| (SEQ ID NO: 477) | |
|---|---|
| 1 | gcccgccctg gccgagcgta gctggcggac cagagccggt agcgaggttg ggagagacgg |
| 61 | agcggacctc agcgctgaag cagaagtccc cggagctgcg gtctccccgc cgcggctgag |
| 121 | ccatgcggct ccctgacctg agaccctgga cctccctgct gctggtggac gcggctttac |
| 181 | tgtggctgct tcagggccct ctggggactt tgcttcctca agggctgcca ggactatggc |
| 241 | tggaggggac cctgcggctg ggagggctgt gggggctgct aaagctaaga gggctgctgg |
| 301 | gatttgtggg gacactgctg ctcccgctct gtctggccac ccccctgact gtctccctga |
| 361 | gagccctggt cgcgggggcc tcacgtgctc ccccagccag agtcgcttca gccccttgga |
| 421 | gctggctgct ggtggggtac ggggctgcgg ggctcagctg gtcactgtgg gctgttctga |
| 481 | gccctcctgg agcccaggag aaggagcagg accaggtgaa caacaaagtc ttgatgtgga |
| 541 | ggctgctgaa gctctccagg ccggacctgc ctctcctcgt tgccgccttc ttcttccttg |
| 601 | tccttgctgt tttgggtgag acattaatcc ctcactattc tggtcgtgtg attgacatcc |
| 661 | tgggaggtga ttttgacccc catgcctttg ccagtgccat cttcttcatg tgcctcttct |
| 721 | cctttggcag ctcactgtct gcaggctgcc gaggaggctg cttcacctac accatgtctc |
| 781 | gaatcaactt gcggatccgg gagcagcttt tctcctccct gctgcgccag gacctcggtt |
| 841 | tcttccagga gactaagaca ggggagctga actcacggct gagctcggat accaccctga |
| 901 | tgagtaactg gcttccttta aatgccaatg tgctcttgcg aagcctggtg aaagtggtgg |
| 961 | ggctgtatgg cttcatgctc agcatatcgc ctcgactcac cctcctttct ctgctgcaca |
| 1021 | tgcccttcac aatagcagcg gagaaggtgt acaacacccg ccatcaggaa gtgcttcggg |
| 1081 | agatccagga tgcagtggcc agggcggggc aggtggtgcg ggaagccgtt ggagggctgc |
| 1141 | agaccgttcg cagttttggg gccgaggagc atgaagtctg tcgctataaa gaggcccttg |
| 1201 | aacaatgtcg gcagctgtat tggcggagag acctggaacg cgccttgtac ctgctcgtaa |
| 1261 | ggagggtgct gcacttgggg gtgcagatgc tgatgctgag ctgtgggctg cagcagatgc |
| 1321 | aggatgggga gctcacccag ggcagcctgc tttcctttat gatctaccag gagagcgtgg |
| 1381 | ggagctatgt gcagaccctg gtatacatat atggggatat gctcagcaac gtgggagctg |
| 1441 | cagagaaggt tttctcctac atggaccgac agccaaatct gccttcacct ggcacgcttg |
| 1501 | cccccaccac tctgcagggg gttgtgaaat tccaagacgt ctcctttgca tatcccaatc |
| 1561 | gccctgacag gcctgtgctc aaggggctga cgtttaccct acgtcctggt gaggtgacgg |
| 1621 | cgctggtggg acccaatggg tctgggaaga gcacagtggc tgccctgctg cagaatctgt |
| 1681 | accagcccac agggggacag gtgctgctgg atgaaaagcc catctcacag tatgaacact |
| 1741 | gctacctgca cagccaggtg gtttcagttg ggcaggagcc tgtgctgttc tccggttctg |
| 1801 | tgaggaacaa cattgcttat gggctgcaga gctgcgaaga tgataaggtg atggcggctg |
| 1861 | cccaggctgc ccacgcagat gacttcatcc aggaaatgga gcatggaata tacacagatg |
| 1921 | taggggagaa gggaagccag ctggctgcgg gacagaaaca acgtctggcc attgcccggg |
| 1981 | cccttgtacg agacccgcgg gtcctcatcc tggatgaggc tactagtgcc ctagatgtgc |
| 2041 | agtgcgagca ggccctgcag gactggaatt cccgtgggga tcgcacagtg ctggtgattg |
| 2101 | ctcacaggct gcaggcagtt cagcgcgccc accagatcct ggtgctccag gagggcaagc |
| 2161 | tgcagaagct tgcccagctc caggagggac aggacctcta ttcccgcctg gttcagcagc |
| 2221 | ggctgatgga ctgaggcccc agggatactg ggccctcttc tcaggggcgt ctccaggacc |
| 2281 | cagagctgtt cctgctttga gtttccctag agctgtgcgg ccagatagct gttcctgagt |
| 2341 | tgcaggcacg atggagattt ggacactgtg tgcttttggt ggggtagaga ggtggggtgg |
| 2401 | ggtggggtgg gggctgtctg tgtccaggaa acttaattcc ctggtgacta gagctttgcc |
| 2461 | tggtgatgag gagtattttg tggcataata catatatttt aaaatatttt ccttcttaca |
| 2521 | tgaactgtat acattcatat agaaaattta gacaatataa aaaagtacaa agaagaaaag |
| 2581 | taaaagtacc cattgtttca cttcctggag ataaccatag ttgctatttt gctgcctgtc |
| 2641 | ccatcagtcg tttatctgtt gtttgagata gaaattaacc aaaaatgaca taaatattca |
| 2701 | tgagattgcc ttcctatatc cttccttgtt cctaccagtg tctgctattt tgaagaagct |
| 2761 | agggtctgga gggacagaga acagttccct gattaacagt attaatagcg acattggtaa |
| 2821 | cagctaccat ttatagagtt ttaatgggag taggagctat gctaagtgtt tttcatgtat |
| 2881 | tatcgttttt aatcattatc cccaacccta tgaggttggt tattatcccc attttacaga |
| 2941 | tgaggaaact gaagctcaaa gaggctcaat gactttccca aggtggtcgt agtggtggag |
| 3001 | ttggagtttg aacacaggcc tgaccctaga gtccacaccc tgacccaatc aattatattg |
| 3061 | catcttgggt ccataaaccc taatccataa tcccatcaag aaaagctctg ctgctcttag |
| 3121 | ctctaaataa ttcagaatct attctcttct ctccagtccc gttgttatag tcttcactca |
| 3181 | tagacttaag atgatcccat caccagagag gtttctctac cattagcttc cctcttccgg |
| 3241 | ccattcttca caaagtcatt tttctaaatt ctgtgtcaca tacgatgatg gcatttctgg |
| 3301 | aaattccttc aggtgctctc aagccctgct gcagagatcc ttttcagagc acacactgtt |
| 3361 | ccagcccatc tgtctcaccc tctcctgttg tatccagctc cacgacaaac ttctgccttc |
| 3421 | cccaacacct ttgtgccttt gcatatggtg ttttcttgcc cattttctgc tcgactcgcc |
| 3481 | cctgattttc aagttcaaga cttaactcag ggttcaggtc ttccaggagg ccttacttat |
| 3541 | gtcgtcagtc tggggaactc tccatgtgct tctatcactg tgcggttacc tctttcacag |
| 3601 | cccttttaaa gttctatctt ccctttccca ccttttttga ccttccacta gaccatgagc |
| 3661 | acctgggcgg aaagccatat atcttattaa gctttatatc tgctacctgg ccgagggcct |
| 3721 | aattcatagt ggagaataaa tagtcaattg aataaatgaa taaatatctc caccatcgta |
| 3781 | ctaatcttaa tcctccctgc ccactcccac cactgaaaat gcaacattgt acacatcact |
| 3841 | ggttgttggg agggacttac cttggaaagt tgctattcta ggaaagagaa accttcatat |
| 3901 | tcctggaaac agcaggtagt ttccagtgct ggcaatgaat tccccagaac tgctgttttg |
| 3961 | gattttttct tgcctggcag ctgttgggag cagggtgcag tgaggatggg gtgagagtgg |
| 4021 | gcagtttctt gtgcagattt gcctttcttt catcctgggg ctgacttgca gctccacacc |
| 4081 | catccatctc tcaaatttca cagagggtaa aataggcatt tggagagaaa gaactctggc |
| 4141 | ctgattcctt tctctcccac aaatgtcctt tattcataaa acaggaataa taattcctgt |
| 4201 | atctcccaac tacatggaag ctgcagccct cacagaagaa gatgatctga gaaattcttt |
| 4261 | gatttcctca gtacagttat acccatgcat cataatactt taagcctgga aggcatctta |
| 4321 | aaaataatgc aacagtcaaa cctaatttta cagagaaact gacatgaaat cacgcagcta |
| 4381 | atcatgataa agctgggtgg aaaacttatc ttgatgggca gtacaggaag atgcagtaga |
| 4441 | ccttaagatg tcctgaaagt ttcttatctc aggggaaact cccaggtagg ctttatgtca |
| 4501 | gggacacaga aaaatgctcc ctgaaagtca aaatattcgg gctagacaga caaattcctg |
| 4561 | taagtgtggt ttgtctggga accacagatg tcactaatcc tggtttgctc cagagttctt |
| 4621 | tttgttcact cctacccccc atcaccattt gattgatctc cttaccctgt aatttcccct |
| 4681 | tcttgtcgct tacctgcagt atctttccca cccaggcatg ccttattctt tctaaaggaa |
| 4741 | agtatgaatg gagaggggaa agcttgggaa actgatagat ttccttggat gccaaaacac |
| 4801 | ctccatagcc tgtctgcccg gccctatgtg gaaacagcat tgagtttcaa gtcctttatg |
| 4861 | cctccaccca gggatagcca cttgtaatcc acatggcaat tgtgaaacaa gcaggaaatg |
| 4921 | cgtaattgtc agaattttgt ggggaaagga ctagggaata aggaaaacaa agatcttcct |
| 4981 | tgtgttttag agctgtcagc tagaggagca cctgcttgag tctgatgcca tctaatggtc |
| 5041 | ccagaagaaa ctgggttttg aacctagagt tccatggact cttaggaatt agactactac |
| 5101 | tactactaag cattcactgg tgcttactat gtgctattgc tgtgccaagt atctgaaacc |
| 5161 | tgtcttctta ccttattttt caagataatt ctatgtggca ggtattacta tctcaattct |
| 5221 | aagagtgaga aaatggagtt ttagaaacat ttactaactt gcctgggtca catagctaag |
| 5281 | gaagaggtgg acttgcccag ctttgcataa aactcctcaa aagagttgcc tatactccct |
| 5341 | gactccactt atcttcctac tatcctcttt ttaaaatata ttatttattt atttaaataa |
| 5401 | gcaatatatg aatgtggttt gaaattcaaa agacacaaag aagtatacag aggaaagcct |
| 5461 | cactctcaat ccttctcaag gtttgctaat tcctcttgca taggcaatcc gttcttccag |
| 5521 | ctttgtgttt atctttccag agaagtttac tgtgtattaa gcaaatatgt atatctttat |
| 5581 | tcttgctcag tattttcgca aacagcagct gtctaagttc actgttctga actttatttt |
| 5641 | ttaaattaaa aatatatggc tatgtagtat tctatttta. |

The TAP2 gene corresponds to Ensembl: ENSG00000204267.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C.

In some instances, contacting a cell and/or polynucleotide with a UGI and a base editor prevents base excision repair which changes the U back to a C. An exemplary UGI comprises an amino acid sequence as follows: >splP14739IUNGI_BPPB2 Uracil-DNA glycosylase inhibitor MTNLSDIIEKETGKQLVIQESILMLPEEVEEVIGNKPESDILVHTAYDESTDENVMLLTSD APEYKPWALVIQDSNGENKIKML (SEQ ID NO: 231). In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid sequence into a cell, resulting in a transformed cell. Vectors include plasmids, transposons, phages, viruses, liposomes, lipid nanoparticles, and episomes. “Expression vectors” are nucleic acid sequences comprising the nucleotide sequence to be expressed in the recipient cell. Expression vectors contain a polynucleotide sequence as well as additional nucleic acid sequences to promote and/or facilitate the expression of the introduced sequence, such as start, stop, enhancer, promoter, and secretion sequences, into the genome of a mammalian cell. Examples of vectors include nucleic acid vectors, e.g., DNA vectors, such as plasmids, RNA vectors, viruses or other suitable replicons (e.g., viral vectors). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Certain vectors that can be used for the expression of antibodies and antibody fragments of some aspects and embodiments herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of some aspects and embodiments herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 is a schematic depicting the structure of the peptide loading complex (PLC), which includes beta-2 microglobulin (β2M), Transporter Associated with Antigen Processing I (TAP1), Transporter Associated with Antigen Processing II (TAP2), and Tapasin. The PLC participates in HLA class-I peptide loading and assembly. The PLC functions in selective translocation of class-I specific peptides across the cell membrane.

FIGS. 2A and 2B are histograms depicting surface HLA class-I expression of T cells that were edited using rBE4 and guide RNAs targeting TAP1 (FIG. 2A) and TAP2 (FIG. 2B), P2M positive control, or unedited control. Median fluorescence intensity (MFI) values are reported for HLA class-I expression, and percent (%) loss reflects reduction in MFI relative to unedited T cells.

FIGS. 3A and 3B are graphs depicting surface HLA class-I expression of T cells that were edited using rBE4 and guide RNAs to TAP1 (FIG. 3A) and TAP2 (FIG. 3B), P2M positive control, or unedited control.

FIGS. 4A and 4B depict editing of multiple inhibitory receptors for engineering NK-resistant K562 cells. FIG. 4A are graphs depicting the overexpression of CD47, PD-L1, HLA-G, or HLA-E compared to wild-type control in K562 cells. FIG. 4B are flow cytometry graphs depicting the overexpression of K.Quad of inhibitory receptors (i.e., CD47, PD-L1, HLA-G, and HLA-E) in K562 cells compared to wild-type control. Effector to Target (E:T) ratio indicates ratio of NK cells to K562 cells.

FIGS. 5A-5C depict evaluating persistence of allogeneic CAR-T cells using BLT (bone-marrow, thymus, liver) humanized NSG ((NOD/SCID/IL2γc^{-/-}) mice. FIG. 5A is a schematic depicting the use of T cells isolated from both a syngeneic BLT humanized mouse (iRFP670+ cells) and peripheral blood mononuclear cells (PBMCs) of an allogeneic de-identified human donor (GFP+) to be infused in a 1:1 ratio into recipient syngeneic BLT mice. FIG. 5B provides flow cytometry graphs depicting persistence of peripheral blood allogeneic cells (GFP+) to syngeneic cells (iRFP670+) at one (1), five (5), and fourteen (14) days post-infusion. FIG. 5C provides graphs depicting the ratio of individual CAR⁺ populations (allogeneic GFP+ cells and syngeneic iRFP670+ cells) of the total CAR+ population from three mice (#305, #308, or #309) out of the total CAR⁺ at one (1), five (5), fourteen (14), and twenty-one (21) days post-infusion.

FIGS. 6A and 6B depict the depletion of allogeneic CAR-T cells (GFP+) in multiple tissue types using BLT humanized mice. FIG. 6A are flow cytometry graphs depicting the frequency of syngeneic (iRFP670+) cells (top) and allogeneic (GFP+) cells (bottom) in bone marrow, lymph node, liver and spleen. FIG. 6B is a graph summarizing the frequency of CAR⁺ syngeneic (iRFP670+) cells and allogeneic (GFP+) cells out of the total CD3⁺ cells (%) in bone marrow, lymph node, liver and spleen.

FIG. 7 is a schematic depicting the infusion of allogeneic HLA⁺ and HLA⁻ (β2M knock-out) CAR-T cells at a 1:1 ratio into a recipient BLT mouse.

FIGS. 8A-8F depict recombinant human (rh) NK cell-mediated allorejection of HLA-CAR-T cells in BLT mice. FIG. 8A is a schematic of a timeline for infusing 2.5 μg of rhIL-15 via intraperitoneal injection (IP) and allogeneic HLA⁺ and HLA⁻ (P2M knock-out) CAR-T cells in BLT mice. FIG. 8B provides flow cytometry graphs depicting the presence of NK cells (via CD56 expression) in BLT mice one-week post-treatment with rhIL-15. Untreated mice were used as a control. FIG. 8C provides summary graphs depicting the expansion of NK cells in BLT mice at one and two-weeks post-treatment with rhIL-15 (top). Untreated mice were used as a control (bottom). FIG. 8D provides flow cytometry graphs depicting the frequency of degranulating human donor-derived and BLT mouse-derived rhIL-15 primed NK cells following stimulation with K562 cells (NK cells gated on: Live/Singlets/CD33⁻/CD3⁻/CD19⁻/mCD45⁻/hCD45⁺/CD56⁺). CD107a is a marker used to measure NK cell functional activity. Unstimulated NK cells cultured with media alone or pan-stimulated NK cells with PMA/Ionomycin were used as controls. FIG. 8E is a flow cytometry graph depicting the depletion of HLA- (P2M knock-out) CAR-T cells identified as CD2+HLA-A2-in BLT mice either treated with rhIL-15 or untreated. The graph shows that NK cells rejected the P2M knock-out T cells. FIG. 8F is a graph depicting the number of HLA- (P2M knock-out) CAR-T cells four (4) days post-infusion in BLT mice either treated with rhIL-15 or untreated.

FIG. 9 presents histograms demonstrating the knock-out of CD58 in cells using Cas9 (nuclease-active SpCas9), ABE8.20m, or rBE4 in combination with the indicated gRNAs (i.e., CD58.1, CD58.2, or CD58.3). “No EP” indicates a negative control where cells were not electroporated. “rBE4+TSBTx026” indicates a control in which cells were exposed to rBE4 in the presence of a negative-control gRNA sequence (TSBTx026). Surface expression of CD58 was measured using flow cytometry. The y-axis represents fluorescence intensity and the x-axis represents level of CD58 expression in the cells. In FIG. 9, “control gRNA” refers to a TRAC-specific guide RNA (target sequence: TTCGTATCTGTAAAACCAAG (SEQ ID NO: 671; PAM: AGG, and “No EP” refers to a negative control not subjected to base editing or electroporation (EP).

FIGS. 10A-10G provide bar graphs showing the frequency of on-target base editing (y-axis), expressed as a percentage (%), at the indicated target genes (indicated along y-axis) using the indicated gRNAs (x-axis) and base editors (ABE and CBE). The ABE base editor introduced an A to G alteration to a target gene and the CBE base editor introduced a C to T alteration to a target gene. The target genes were NLRC5 (CITA; FIG. 10A), TAPBP (Tapasin; FIG. 10B), PDIA3 (ERp57; FIG. 10C), CD155 (FIG. 10D), MICA (FIG. 10E), MICB (FIG. 10F), and CD48 (FIG. 10G). In FIGS. 10A-10G, CBE indicates rBE4 mRNA600, and ABE indicates ABE8.20. The guide RNAs identified along the x-axis of FIGS. 10A-10G are provided in Table 1A. The term “PDIA3 (ERp57)” represents “protein disulfide isomerase family A member 3,” the term “TAPBP” represents “TAP binding protein”, the term “NLRC5 (CITA)” represents “NLR family CARD domain containing 5 (class-I transcriptional activator),” the term “MICA” represents “MHC class I polypeptide-related sequence A,” the term “MICB“represents” MHC class I polypeptide-related sequence B,” the term “Nectin-2” represents “nectin cell adhesion molecule 2,” and the term “ULBP” represents “UL16 binding protein 1-6.”

FIG. 11 provides a schematic of a generic HLA single-chain trimer. In FIG. 11 “GS” represents a poly-Gly/Ser linker that intervenes individual elements of the single-chain trimer.

FIG. 12 provides flow cytometry plots (upper panel) and corresponding histograms (lower panel) demonstrating that the HLA-E single-chain trimers and dimers were secreted by anti-CD4 based-CAR-T cells. The term “UTD” indicates the untransduced control. From left to right, the plots and histograms correspond to the untransduced control (UTD) cells, construct BTx_CM193, construct BTx_CM211, construct BTx_CM212, and construct BTx_CM213. The polypeptide sequences for each construct is provided in Table 19. In the histograms (lower panel of FIG. 12), the x-axis corresponds to level of secretion of each HLA-E construct. The anti-CD4 based-CAR-T cells did not express a function β2M.

FIG. 13 provides a flow cytometry histogram demonstrating detection of a PE-labeled HLA-A2 tetramer using M-280 streptavidin beads conjugated to an anti-β2M antibody (αβ2M).

FIGS. 14A and 14B provide plots showing protection of β2M knock-out CAR-T cells secreting HLA-E single-chain trimers from NK cell-mediated killing. The plots indicate the degree of specific lysis (y-axis) of β2M knock-out T cells transduced with BTx_CM193 (FIG. 14A) and BTx_CM211 (FIG. 14B) relative to control untransduced (UTD) β2M knock-out T cells at the indicated effector-to-target (E:T) ratios (x-axis) 48 hours post-coculture.

FIGS. 15A-15C provide a schematic, histograms, and a plot showing that B2M knock-out overcame in vitro allogeneic T cell mediated killing. FIG. 15A provides a schematic showing the experimental setup for a mixed leukocyte reaction to evaluate the impact of B2M knock-out on T cell mediated killing. FIG. 15B provides a set of flow cytometry histograms showing that B2M knock-out protected cells from T cell mediated killing. FIG. 15C provides a plot showing that B2M knock-out protected cells from T cell modified killing. In FIGS. 15B and 15C “E:T” represents the ratio of effector to target cells, and AF647 represents “Invitrogen Alexa Fluor 647 dye fluorescence.”

FIGS. 16A-16D provide schematics, histograms, and a plot showing that B2M knock-out increased sensitivity to in vitro NK cell mediated killing. FIG. 16A provides a schematic showing the experimental setup for a mixed leukocyte reaction to evaluate the impact of B2M knock-out on NK cell mediated killing. FIG. 16B provides a set of flow cytometry histograms showing that B2M knock-out cells were susceptible to NK cell mediated killing. FIG. 16C provides a plot showing that B2M knock-out cells were susceptible to NK cell-mediated killing. In FIGS. 16B and 16C “E:T” represents the ratio of effector to target cells, and AF647 represents “Invitrogen Alexa Fluor 647 dye fluorescence.” FIG. 16D provides a schematic showing how knockout of B2M increases susceptibility of a modified T cell to killing by the NK cells of a recipient subject. In FIG. 16D, “KIRs” represents “killer Ig-like receptors,” NKG2A represents “CD94/NK group 2 member A,” and LIR-1 represents “leukocyte Ig-like receptor 1.” Without being bound by theory, it is believed that knock-out of B2M in a T cell leads to a deficiency in the T cell of HLA class-I surface expression, which removes a critical NK cell inhibitory signal via HLA class-I interaction with NK cell receptors such as KIRs, NKG2A, or LIR-1.

FIG. 17 provides a plot showing that BLT mice recapitulated key features of allorejection. The plot shows that, in the BLT mice, WT (“wild-type”) CAR-T cells (i.e., unedited cells without B2M knock-out) were killed during the period of the experiment and B2M knock-out CAR-T cells persisted. Therefore, B2M knock-out prevented host T cell allorejection. As a control experiment, it was determined that the wild type CAR-T cells persisted in mice devoid of endogenous T cells (see arrow in the figure). The y-axis represents the concentration of CAR-T cells in peripheral blood.

FIGS. 18A and 18B provide a schematic and a set of plots showing that HLA-E single chain trimer (SCT) conferred protection against in vitro NK cell mediated killing. FIG. 18A provides a schematic showing the experimental setup for a mixed leukocyte reaction to evaluate the impact of membrane-bound HLA-E single-chain trimer (SCT) expression in a T cell on NK cell mediated killing of the T cell. FIG. 18B provides plots showing that expression of the HLA-E SCT conferred protection against in vitro NK cell mediated killing. In FIG. 18B, the x-axis shows the ratio of effector to target cells (E:T).

FIG. 19 provides a schematic listing targets for a base editing strategy to overcome recipient NK cell rejection of B2M knock-out CAR-T cells. In FIG. 19 “BE” represents “base editing.

FIGS. 20A and 20B provide schematics showing domain architectures for membrane-anchored (“Anchored”) and soluble HLA-E single-chain trimers (SCTs), single-chain dimers (SCDs), and monomers suitable for expression in T cells to reduce allorejection by tuning HLA class-I expression independent of B2M knock-out. In FIG. 20A, the unlabeled, light-grey domain represents a loading peptide (LP) domain. In FIGS. 20A and 20B “LP” represents “loading peptide,” “ECD” represents “extracellular domain,” “TM” represents “transmembrane domain,” unlabeled white domains represent the extracellular domain of HLA-E, unlabeled light grey domains represent loading peptide domains, unlabeled medium-grey domains represent a G5 tail, “ΔTM” represents a change in position of the “transmembrane domain,” unlabeled dark grey domains represent a transmembrane domain, and a dotted box represents a deleted domain.

FIGS. 21A-21E provide histograms and plots showing that base editing of the indicated peptide loading genes (i.e., TAP1, Tapasin, TAP2, ERp57, and CITA) using the indicated editors (i.e., Cas12b or ABE) inhibited HLA class-I surface expression in T cells. In FIGS. 21A-21E the black curves represent HLA-ABC expression in edited cells and the shaded curves represent HLA-ABC expression in unedited cells. In FIGS. 21A-21E, the dots in the plot represent HLA expression relative to unedited cells (“UE”) resulting from base-editing using various gRNA's (x-axis). Guide RNA's used to edit the cells are listed in Table 1A.

FIG. 22 provides a schematic of an expression construct used to screen for sgRNA sequences suitable for base editing of the genome of a T cell using CBE to reduce expression of B2M/MHL I expression for allogeneic persistence. The genes targeted by the library included B2M, TAP1, TAP2, TAPBP, and/or TAPBPL. The library size was 2845, including 113 essential gene guides, and 120 non-targeting guides. Cells were transduced with the library constructs and sorted for B2M− T cells using flow cytometry to then identify which sgRNA sequences were enriched in the B2M− T cells. The promoter for the sgRNA sequence was U6 and the promoter for the CAR was EF1α. In FIG. 22, LTR represents a “long terminal repeat” sequence, T2A represents a self-cleaving peptide, CD4 eCD represents a co-stimulatory domain, 4-1BB represents a co-stimulatory domain that promotes T cell survival, and CD3ζ represent the domains of a chimeric antigen receptor, WPRE represents a Woodchuck Hepatitis Virus (WHP) Posttranscriptional Regulatory Element, “NGFR” represents an NGFR tag, and “BC” represents a barcode sequence for downstream sequencing analysis.

FIG. 23 provides a series of flow cytometry scatter plots showing the sorting of T cells transfected using libraries designed as described in FIG. 22. The genes whose expression is measured in each row of plots is indicated to the left of each row (i.e., NGFR, TCRa/b, and B2M). The arrows indicate B2M deficient (B2M-) cells that were sorted.

FIG. 24 provides scatter plots providing an overview of how T cells transfected using the libraries designed as described in FIG. 22 were sorted for B2M deficient cells to yield a sorted population. About 200M T cells were sorted and around 400 k cells were recovered in the sorted population.

FIG. 25 provides a plot showing enrichment for different guide sequences in the sorted population obtained according to the T cell sorting strategy described in FIG. 24. Enriched guides (see also Table 20) had higher values on the y-axis (i.e., sorted cell counts). The y-axis represents “sorted cell counts” and the x-axis represents “library input cell counts.” The dotted line represents 3 standard deviations multiplied by the mean value for all sorted cell counts.

FIG. 26 provides a plot showing a MAGeCK analysis to obtain p-values for guide RNA sequences enriched in the sorted population of T cells obtained using the sorting strategy described in FIG. 24. The guide b2m_825 (TSBTx845) was a previously-validated guide. Enriched guides included those targeting TAP1, TAP2, TAPBP (tapasin), and B2M. The Y-axis in FIG. 26 represents a logarithmic scale (−Log 10) and, to shorten the y-axis, there is a discontinuity in the scale between the values 3 and 10. Sequences for guide RNAs identified in FIG. 26 are provided in Table 1A.

FIG. 27 represents a multiple sequence alignment of human HLA genes. The dark regions in the alignments represent variable regions and the light-grey areas represent conserved regions. The arrows represent sites targeted by designed guide RNA's. Guide RNA's were designed to be compatible with ABE and/or CBE and to target the start site, a splice acceptor site, or a splice donor site. Guide RNA's were also designed to be compatible with Cas12b and to target exon conserved regions.

FIGS. 28A and 28B provide bar plots and a shaded chart showing the identification of gRNAs (indicated on the x-axis) that facilitated base editing resulting in a reduction in HLA class-I surface expression in T cells. In FIG. 28A, the rightmost bar in each graphs represents unedited cells and the y-axis in each graph represents frequency of surface protein expression for HLA-A or HLA-B, or percent HLA-C expression relative to unedited T cells. B2M knock-out reduced HLA-A, -B, and -C expression. The guide TSBTx4190 (g850) specifically knocked out HLA-A*02, which had a 1 nt mismatch from other HLA-A alleles (see dashed box on left). The TSBTx4200 (g860) guide specifically knocked out HLA-B. The TSBTx4193 (g853) and TSBTx4194 (g854) guides overlapped +1 nt targeting SD exon 4 (α3 domain). The guide RNAs are provided in Table 1A FIG. 28B provides the genotypes for three of the four donors whose cells were edited in the experiment. The guide RNAs targeting HLA class-I alleles were screened in activated T cells from 4 independent donors.

FIG. 29 presents a set of histograms showing the impact of base editing using guide RNAs TSBTx4190 (g850) and TSBTx4200 (g860) individually or together in combination with ABE8.20m on expression of HLA-A2, HLA-Bw6 (HLA-B), HLA-C, and pan HLA-E in edited primary T cells. The edited cells were from donor D270202 (see FIG. 28B). The guides TSBTx4190 and TSBTx4200 were designed to specifically disrupt HLA-A and HLA-B expression, respectively. As a control, a population of cells was also edited to knock-out expression of B2M.

FIGS. 30A and 30B provide a set of flow cytometry scatter plots and a plot showing that targeted HLA class-I knock-out using base editing mitigated T cell allorejection in a mixed lymphocyte reaction (MLR). Cells were labeled using carboxyfluorescein succinimidyl ester (CFSE) (measured on x-axis of FIG. 30A) or CellTrace™ Far Red (CTFR) (measured on y-axis of FIG. 30A). The labels to the left of the scatter plots of FIG. 30A represent the effector to target cell ratio (E:T) for the corresponding row of scatter plots. The impact on T cell allorejection was determined for CFSE-labeled on-target cells including HLA+(unedited), HLA-A knock-out (edited using guide RNA TSBTx4190), HLA-B knock-out (editing using guide RNA TSBTx4200), and HLA-A and HLA-B knock-out (“Dual KO”) cells relative to CTFR-labeled off-target B2M knock-out T cells by HLA class-I mismatched effector T cells. Data is shown 48 hours post-culture. In FIG. 30B “E:T” represents the ratio of effector to target cells.

FIGS. 31A and 31B provide a set of flow cytometry scatter plots and a plot showing that HLA-B knock-out resulted in protection against NK cell lysis. Cells were labeled using carboxyfluorescein succinimidyl ester (CFSE) (measured on x-axis of FIG. 31A) or CellTrace™ Far Red (CTFR) (measured on y-axis of FIG. 31A). The labels to the left of the scatter plots of FIG. 30A represent the effector to target cell ratio (E:T) for the corresponding row of scatter plots. The impact on NK cell lysis was determined for CTFR-labeled on-target cells including B2M knock-out (KO), HLA-A knock-out (edited using guide RNA TSBTx4190/g850), HLA-B knock-out (editing using guide RNA TSBTx4200/g860), and HLA-A and HLA-B knock-out (“Dual KO”) cells relative to CFSE-labeled off-target unedited cells (HLA+) T cells by NK cells. Data is shown 48 hours post-culture. In FIG. 31B “E:T” represents the ratio of effector to target cells.

FIG. 32 provides a plot showing that specific HLA-ABC knock-out prevented T cell-mediated allorejection in vivo. BLT mice were co-infused with an equal mixture of unedited (HLA+), B2M knock-out, and HLA-ABC knock-out T cells. All T cells were edited for TRAC and CIITA. HLA-ABC knock-out T cells were additionally multiplex edited with the following guide RNAs: TSBTx4190, which targets HLA-A, TSBTx4201, which targets HLA-B, and TSBTx4208, which targets HLA-C. The graph shows peripheral blood concentration of each T cell population.

FIGS. 33A-33D provide flow cytometry histograms and a plot showing that HLA deficient CAR-T cells evaded T cell-mediated allorejection in vitro. FIGS. 33A and 33B show HLA class-I expression (FIG. 33A). and HLA class-II (FIG. 33B) surface expression in T cells after base editing with rBE4 or ABE8.20m of the indicated genes. FIGS. 33C and 33D shows results from an in vitro T cell Mixed Leukocyte Reaction using target T cells (Mock Edit and B2M knock-out) co-cultured with HLA mismatched effector cells. The B2M KO cells, which were deficient in HLA-ABC expression (shaded curve in FIG. 33C and lower curve in FIG. 33D) evaded T cell killing. In FIGS. 33A-33D, the mock edited cells correspond to the dashed curves or lines.

FIG. 34 provides a schematic, and a set of flow cytometry scatter plots describing a humanized BLT mouse model used to evaluate allorejection evasion strategies in vivo. The schematic describes the generation of BLT mice. Steps for preparation of the BLT mice include 1) sub-lethal whole-body irradiation, 2) human fetal thymus and liver transplant, and 3) injection of fetal-liver CD34+ hematopoietic stem cells (HSCs). The flow cytometry scatter plots show the identification of human immune cells in peripheral blood 12 weeks post-tissue implantation.

FIGS. 35A-35C provide flow cytometry histograms, a schematic, and a flow cytometry scatter plot showing that HLA deficient CAR-T cells overcame in vivo T cell-mediated allorejection. FIG. 35A provides histograms confirming reduced polypeptide expression in TRAC knock-out (KO) or the Triple knock-out (TKO) TRAC, B2M, and CIITA allogeneic CAR-T cells relative to unedited cells. In FIG. 35A, the dashed curves represent the TRAC-KO CAR-T cells, the dark grey shaded curves represent the TKO CAR-T cells, and the light-grey shaded curve represents mock edited cells. In FIG. 35A, the numbers from top-to-bottom in each plot moving from left-to-right (i.e., TRAC, B2M, CIITA, respectively) represent the percent of cells measured to be deficient in expression of the indicated polypeptide in 1) mock edit cells, TRAC-KO cells, or TKO cells, 2) TRAC-KO cells or TKO cells, and 3) in TRAC-KO cells or TKO cells. FIG. 35B provides a schematic showing an experimental design where BLT mice were co-infused with a 1:1 ratio of TRAC-KO and TKO CAR-T cells. FIG. 35C provides scatter plots showing frequency of peripheral TRAC-KO CAR-T cells (black) and TKO CAR-T cells (gray) post-infusion.

FIG. 36 provides a schematic showing how base editing may be used to introduce a stop codon to a gene or to disrupt a splicing motif (e.g., a splice acceptor site, or a splice donor site).

FIG. 37 provides a plot showing results from a natural killer (NK) cell mixed leukocyte reaction demonstrating that T cells edited using pan HLA class-I sgRNAs (i.e., TSBTx4193 and TSBTx4194) were protected from NK cell lysis in vitro, whereas B2M knock-out cells were susceptible to lysis. Primary human NK cells were co-cultured with T cells base-edited using a base editor in combination with a guide targeting pan HLA class-I (i.e., RNA TSBTx4193 or TSBTx4194) or a guide targeting beta-2-microglobulin. The cells were co-cultured for 48 hours at the E:T ratios indicated on the x-axis of FIG. 37 prior to assessment of specific cell lysis. In FIG. 37 “E:T ratio” represents the ratio of effector cells (E), which were NK cells, to target cells (T), which were the base-edited T cells.

FIG. 38 provides a plot showing results from a natural killer (NK) cell mixed leukocyte reaction demonstrating that T cells edited using HLA class-I sgRNAs (i.e., TSBTx4193 and TSBTx4194) showed increased resistance to NK cell lysis relative to unedited HLA+ unedited T cells. Primary human NK cells were co-cultured with unedited HLA class-I mismatched (HLA+) T cells or with T cells base-edited using a base editor in combination with a guide targeting pan HLA class-I (i.e., RNA TSBTx4193 or TSBTx4194). The cells were co-cultured for 48 hours at the E:T ratios indicated on the x-axis of FIG. 37 prior to assessment of specific cell lysis. In FIG. 38 “E:T ratio” represents the ratio of effector cells (E), which were NK cells, to target cells (T), which were the base-edited or HLA+ T cells.

FIG. 39 provides a bar graph showing that was the dominant population of T cells resulting from base editing of the T cells using the guide RNA TSBTx4193 expressed only HLA-C. A population of allogeneic T cells was base edited using TSBTx4193 and then stained with antibodies against HLA-A2, HLA-Bw6, and HLA-C followed by flow cytometry. The bar graph of FIG. 39 shows the expression pattern of HLA class-I on the cells. In FIG. 39, HLA class-I expression percent is expressed relative to unedited cells.

FIG. 40 provides a plot showing that allogeneic T cells expressing HLA-ABC+ or HLA-C were protected from NK cell-mediated rejection in IL-15 primed BLT mice, whereas HLA-ABC negative or HLA class-I deficient T cells were not. A population of base-edited allogeneic T cells was infused into IL-15 primed BLT mice. The population of base-edited allogeneic T cells was prepared using a base editor and the guide RNA TSBTx4193. The plots of FIG. 40 show the number of allogeneic T cells with the immunophenotypes HLA-ABC+, HLA-C+, or HLA-ABC− per microliter of blood at 1 and 7 days post-infusion. Those base-edited cells deficient in expression of HLA-A and HLA-B and expressing HLA-C were protected from NK cell-mediated rejection in the IL-15 primed BLT mice, as were cells expressing HLA-ABC.

FIG. 41 provides a plot demonstrating that T cells expressing HLA-C+ and deficient in expression of HLA-A and HLA-B were resistant to in vivo T cell-mediated rejection in IL-15 primed BLT mice, whereas HLA-ABE+ allogeneic T cells were not. A population of allogeneic T cells was base edited using a base editor and the guide RNA TSBTx4193. The base-edited population of allogeneic T cells was then infused into IL-15 primed mice. The plot of FIG. 41 shows the number of allogeneic T cells with the immunophenotypes HLA-ABC+, HLA-C+, or HLA-ABC− per microliter of blood at 1, 7, and 14 days post-infusion. About 50% of allogeneic T cells expressing HLA-C+ were resistant to rejection.

FIGS. 42A-42C provide a schematic, heat maps, and a bar graph showing that base editing using some guide RNAs designed to target TAP2, TAP1, TAPBP, or B2M resulted in downregulation of B2M surface-expression but not HLA class I surface-expression. FIG. 42A provides a schematic showing the interaction between B2M, HLA class I (HLA-I) heavy chain polypeptides, and components of the peptide loading complex containing Tap2, Tap1, Tapasin, ERp57, and TAPBPL. FIG. 42B provides flow cytometry heatmaps showing that base editing using a base editor in combination with guide 65 (see guide b2m_629 listed in Table 1A) resulted in downregulation of B2M expression. The numbers in the squared-off regions of the heatmaps of FIG. 42B indicate the percent of cells counted that did not surface-express B2M.

FIG. 42B provides a bar graph showing that guides 47-68 resulted in downregulation of B2M surface-expression but had less or no effect on expression of HLA-A, -B, and —C surface-expression. In FIG. 52B, the B2M guide RNA corresponds to TSBTx845 (see Table 1A). In FIGS. 42B and 42C, guide 47 corresponds to Tap2_5, which targets TAP2; guide 48 corresponds to Tap1_93, which targets TAP1; guide 49 corresponds to Tap2_4, which targets TAP2; guide 50 corresponds to Tap1_139, which targets TAP1; guide 51 corresponds to Tapbp_18, which targets TAPBP; guide 52 corresponds to Tap2_137, which targets TAP2; guide 53 corresponds to Tapbp_64, which targets TAPBP; guide 54 corresponds to Tap1_161, which targets TAP1; guide 55 corresponds to Tap1_454, which targets TAP1; guide 56 corresponds to Tap1_485, which targets TAP1; guide 57 corresponds to Tapbp_5, which targets TAPBP; guide 58 corresponds to b2m_629, which targets B2M; guide 59 corresponds to b2m_630, which targets B2M; guide 60 corresponds to b2m_315, which targets B2M; and guide 61 corresponds to b2m_316, which targets B2M. The guide RNA sequences are provided in Table 1A. In FIGS. 42B and 42C, WT (“wild type”) cells were unedited T cells and the “Positive Control” or “B2M” cells were edited using the guide TSBTx845 known to be effective for use in knocking out B2M and HLA-A, -B, and -C expression.

FIGS. 43A-43C provide bar graphs and a collection of flow cytometry histograms showing that multiplex editing was effective in tuning HLA class I expression in T cells. T cells were base editing using guides 47-68 and the indicated combinations thereof, where guide 47 corresponds to Tap2_5, which targets TAP2; guide 48 corresponds to Tap1_93, which targets TAP1; guide 49 corresponds to Tap2_4, which targets TAP2; guide 50 corresponds to Tap1_139, which targets TAP1; guide 51 corresponds to Tapbp_18, which targets TAPBP; guide 52 corresponds to Tap2_137, which targets TAP2; guide 53 corresponds to Tapbp_64, which targets TAPBP; guide 54 corresponds to Tap1_161, which targets TAP1; guide 55 corresponds to Tap1_454, which targets TAP1; guide 56 corresponds to Tap1_485, which targets TAP1; guide 57 corresponds to Tapbp_5, which targets TAPBP; guide 58 corresponds to b2m_629, which targets B2M; guide 59 corresponds to b2m_630, which targets B2M; guide 60 corresponds to b2m_315, which targets B2M; and guide 61 corresponds to b2m_316, which targets B2M. The B2M guide RNA corresponds to TSBTx845 (see Table 1A). The WT (“wild type”) cells were unedited T cells. FIG. 43A provides flow cytometry histograms showing that HLA-A, -B, and —C surface-expression was tuned (i.e., shifted between levels observed for wild-type and B2M knock-out cells) through multiplex editing using guides 56 and 57, which targeted TAP1 and tapasin, respectively. FIGS. 43B and 43C provide bar graphs demonstrating that multiplex base editing using the indicated combinations of guides 47-68 resulted in tuning of HLA-A, -B, and —C surface-expression in base-edited T cells. The combination of guides 56 and 67, which targeted TAP1 and tapasin, respectively, resulted in the largest reduction in HLA-A, -B, and —C surface-expression in base-edited cells. FIGS. 43A-43C demonstrate that reducing HLA-A, -B, and -C expression can require base editing of multiple polypeptide components of the peptide loading complex (see FIG. 42A).

FIGS. 44A and 44B provide a chart and a multiple sequence alignment showing that base editing can be used to knock out expression of HLA-A and HLA-B in immune cells while maintaining expression of HLA-C. FIG. 44A provides a chart listing the percent of HLA-A, -B, and -C alleles analyzed that could be targeted for base editing using guides TSBTx4193 and TSBTx4194. Only 60% of the analyzed HLA-C alleles could be targeted for base editing using the guides, while over 98% of the HLA-A and HLA-B alleles can be targeted using the same guides. FIG. 44B provides a multiple sequence alignment showing that the HLA-C allele HLA-C*02:02:02:01 could be altered using a base editor system containing the guide TSBTx4193 or TSBTx4194, but that allele HLA-C*17:01:01:02 could not be effectively altered using the same guides on account of a mutation in the PAM region. The following sequences are provided in FIG. 44B in order of occurrence from top to bottom:

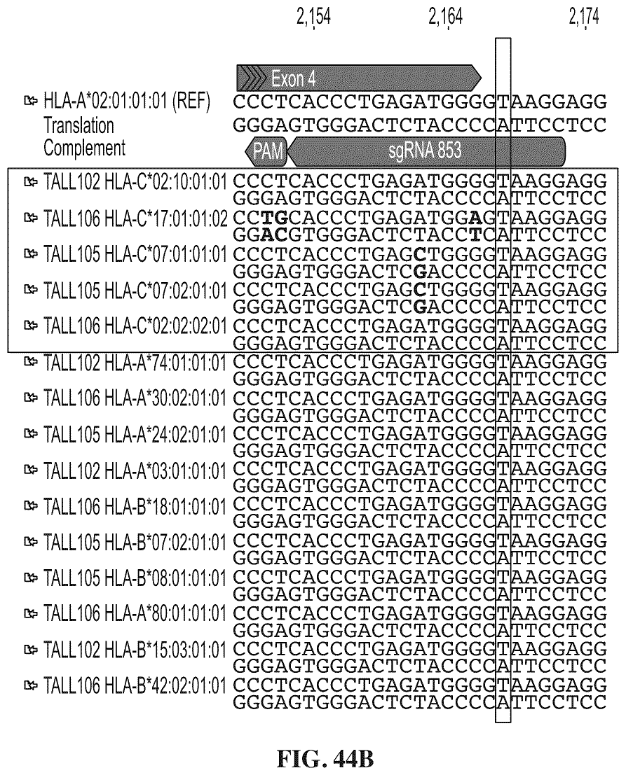

(SEQ ID NO: 2909)
CCCTCACCCTGAGATGGGGTAAGGAGG,
(SEQ ID NO: 2910)
CCTGCACCCTGAGATGGAGTAAGGAGG;
(SEQ ID NO: 2911)
CCCTCACCCTGAGCTGGGGTAAGGAGG.
In FIG. 44B, sgRNA_853
corresponds to TSBTx4193 (853)
(see Table 1A).

### DETAILED DESCRIPTION OF THE INVENTION

The present disclosure features genetically modified allogeneic immune cells (e.g., T- or NK-cells), and methods for producing and using these modified immune cells (e.g., T cells or NK cells).

The invention is based, at least in part, on the discovery that modified immune cell persistence is increased by using base editing technology and/or a nuclease to reduce or eliminate activity and/or surface expression of the following targets in modified immune cells:
1) genes implicated in transcriptional activation of HLA class-I expression (NLRC5/CITA);
2) conserved genomic sequences within the HLA-A, HLA-B, HLA-C loci;
3) genes involved in proper assembly of mature HLA class-I molecules (e.g., TAP1, TAP2, PDIA3/ERp57, and TAPBP); and/or
4) genes encoding proteins that bind NK cell activation receptors,
   including CD155, Nectin-2, CD48, MICA, and MICB; and/or other genes encoding proteins that function in or modulate an immune response, such as components of the peptide loading complex (PLC) (e.g., β2M, TAP1, TAP2, Tapasin) and/or CD58, or regulatory elements thereof, in an allogeneic immune cell (e.g., T- or NK-cell). The modification of immune cells to knock-out or knockdown specific genes is accomplished using a base editor system or nuclease as described herein.

The invention also provides modified immune cells over-expressing ligands of the inhibitory NK2GA receptor (e.g., HLA-E, HLA-G) expressed by Natural Killer (NK) cells. This over-expression provides for the increased persistence of the modified immune cells.

### HLA-G and HLA-E

HLA-G and HLA-E belong to the non-classical HLA-class Ib family. In contrast with classical HLA-Ia molecules (HLA-A, -B, and —C), HLA-E and G negatively regulate the immune response. This allows HLA-E and G expressing cells to avoid recognition and lysis by cytotoxic immune effector cells, such as NK cells. Seven different isoforms of HLA-G are encoded by the same primary mRNA through alternative splicing. Four isoforms (HLA-GT, -G2, -G3, and -G4) retain the transmembrane domain and therefore are membrane-bound, whereas the other three isoforms (HLA-G5, -G6, and -G7) retain intron-4 and lose the transmembrane domain, and are therefore released as soluble molecules. Modified immune cells (e.g., CAR-T cells) expressing soluble or membrane bound HLA-G or -E are expected to show increased persistence in a subject.

### Car-T Cell Therapies

Autologous, patient-derived chimeric antigen receptor-T cell (CAR-T) therapies have demonstrated remarkable efficacy in treating disease. While these products have led to significant clinical benefit for patients, the need to generate individualized therapies creates substantial manufacturing challenges and financial burdens. Allogeneic CAR-T therapies were developed as a potential solution to these challenges, having similar clinical efficacy profiles to autologous products while treating many patients with cells derived from a single healthy donor, thereby substantially reducing cost of goods and lot-to-lot variability. Allogeneic approaches are preferred over autologous cell preparation for a number of situations related to uncertainty of engineering autologous T cells to express a CAR and finally achieving the desired cellular products for a transplant at the time of medical emergency.

However, for allogeneic T cells, or “off-the-shelf” T cells, it is important to carefully negotiate the host's reactivity to the CAR-T cells (HVGD), as well as the allogeneic T cell's potential hostility towards a host cell (GVHD). Additionally, adoptively transferred allogeneic CAR-T cells exhibit poor persistence in vivo due to recipient/host immune-mediated rejection mechanisms. Host-derived alloreactive T cells recognize allogeneic CAR-T cells as “non-self” by binding to peptide complexed with foreign Human Leukocyte Antigen (HLA) on the surface of CAR-T cells. The presence of surface peptide/HLA class-I negatively regulates Natural Killer (NK) cells by engaging inhibitory Killer Ig-Like Receptors (KIRs). As such, the absence of surface HLA on cells leaves them susceptible to NK cell-mediated lysis. Thus, generating HLA-deficient allogeneic CAR-T cells that are below the threshold to activate host-derived alloreactive T cell-mediated rejection and above the threshold to inhibit host-derived NK cells will likely improve allogeneic therapies.

Base editors (BEs) are a class of emerging gene editing reagents that enable highly efficient, user-defined modification of target genomic DNA without the creation of double-stranded breaks (DSBs). In contrast to a nuclease-only editing strategy, concurrent modification of one or more genetic loci by base editing produces highly efficient gene knock-outs with no detectable translocation events. Multiplex editing of genes is likely to be useful in the creation of CAR-T cell therapies with improved therapeutic properties. The methods described herein address known limitations of allogeneic immune cell (e.g., CAR-T cell) products and are a promising development towards the next generation of precision cell-based therapies.

The present disclosure provides modified allogeneic immune cells (e.g., T- or NK-cell) that are less susceptible to NK cell-mediated lysis and are able to overcome host-derived alloreactive T cell-mediated rejection. In some embodiments, the modified allogeneic immune cell described herein is an allogeneic modified CAR-T cell. In some embodiments, the CAR-T cell is an allogeneic T cell that expresses a desired CAR, and can be universally applicable, irrespective of the donor and the recipient's immunogenic compatibility. An allogenic immune cell may be derived from one or more donors. In certain embodiments, the allogenic immune cell is derived from a single human donor. For example, the allogenic T cell may be derived from PBMCs of a single healthy human donor. In certain embodiments, the allogenic immune cell is derived from multiple human donors. In some embodiments, an allogeneic immune cell is generated, as described herein by using gene modification to introduce concurrent edits at one or more genetic loci. In embodiments, an allogeneic immune cell is derived from a stem cell (e.g., an induced pluripotent stem cell (iPSC)). In embodiments, the methods of the disclosure involve editing (e.g., base editing) a stem cell (e.g., an iPSC). A modification, or concurrent modifications as described herein may be a genetic editing, such as a base editing, generated by a base editor. The base editor may be a C base editor or A base editor. As is discussed herein, base editing may be used to achieve a gene disruption, such that the gene is not expressed. A modification by base editing may be used to achieve a reduction in gene expression. In some embodiments base editor may be used to introduce a genetic modification such that the edited gene does not generate a structurally or functionally viable protein product. In some embodiments, a modification, such as the concurrent modifications described herein may comprise a genetic editing, such as base editing, such that the expression or functionality of the gene product is altered in any way. For example, the expression of the gene product may be enhanced or upregulated as compared to baseline expression levels. In some embodiments the activity or functionality of the gene product may be upregulated as a result of the base editing, or multiple base editing events acting in concert. In some embodiments, a base editor and sgRNAs that provide for multiplex editing are introduced in a single electroporation event, thereby reducing electroporation event associated toxicity. Any known methods for incorporation of exogenous genetic material into a cell may be used to replace electroporation, and such methods known in the art are contemplated for use in any of the methods described herein.

The present disclosure provides an alternative means of producing allogeneic immune cells by using base editing technology and/or a nuclease to reduce or eliminate surface HLA class-I expression and/or expression of an NK cell surface activating ligand (e.g., CD58, CD115, CD48, MICA, MICB, Nectin-2, and/or ULBP). In embodiments, base editing technology and/or a nuclease is used to reduce or eliminate activity and/or surface expression of a β2M, TAP1, TAP2, TAPBP, PDIA3, NLRC5, HLA-A, HLA-B, and/or HLA-C polypeptide. In embodiments, base editing technology and/or a nuclease is used to reduce or eliminate surface expression of HLA-A and HLA-B while maintaining surface expression of HLA-C. In embodiments, base editing technology and/or a nuclease is used to knock-out expression of HLA-A and HLA-B (e.g., reduce expression to virtually undetectable levels) while maintaining surface expression of HLA-C. In embodiments, base editing technology and/or a nuclease is used to reduce or eliminate surface expression of HLA-A and HLA-B while maintaining surface expression of HLA-C and B2M. In embodiments, base editing technology and/or a nuclease is used to knock-out expression (e.g., reduce expression to virtually undetectable levels) of HLA-A and HLA-B while maintaining surface expression of HLA-C and B2M. In embodiments, base editing technology and/or a nuclease is used to reduce or eliminate surface expression of HLA-A and HLA-B while maintaining surface expression of B2M. In embodiments, base editing technology and/or a nuclease is used to knock-out expression of HLA-A and HLA-B (e.g., reduce expression to virtually undetectable levels) while maintaining surface expression of B2M. In embodiments, allogeneic immune cells produced according to methods of the present disclosure express B2M and have not been edited to knock-expression of B2M. In some embodiments, at least one or more genes encoding proteins that form the peptide loading complex (PLC) (e.g., β2M, TAP1, TAP2, Tapasin) (“PLC genes”), or regulatory elements of such genes, are modified in an allogeneic immune cell with the base editing compositions and methods provided herein. In some embodiments, the PLC genes comprise or consist of β2M, TAP1, TAP2, and Tapasin. In some embodiments, the PLC genes are TAP1 and/or TAP2.

In some embodiments, the PLC genes (e.g., β2M, TAP1, TAP2, Tapasin), or regulatory elements thereof, are modified in an allogeneic immune cell in combination with one or more modifications in at least one additional gene sequence or regulatory element thereof. In some embodiments, the additional gene sequence or regulatory element is selected from TCRα Chain (TRAC), Cluster of Differentiation 58 (CD58), and Class II, Major Histocompatibility Complex Transactivator (CIITA). In some embodiments, one or more of β2M, TAP1, TAP2, and/or Tapasin encoding genes are modified in an allogeneic immune cell in combination with one or more modifications in TRAC, CD58, and/or CIITA encoding genes.

In some embodiments, PLC genes (e.g., β2M, TAP1, TAP2, Tapasin), or regulatory elements thereof, are modified in an allogeneic immune cell in combination with the overexpression of one or more inhibitory receptors. In some embodiments, the inhibitory receptors are selected from Human Leukocyte Antigen-E (HLA-E), Human Leukocyte Antigen-G (HLA-G), Programmed Death Ligand 1 (PD-L1), Cluster of Differentiation 47 (CD47), and/or Cluster of Differentiation 58. In some embodiments, one or more of β2M, TAP1, TAP2, and/or Tapasin are modified in an allogeneic immune cell in combination with the overexpression of one or more of HLA-E, HLA-G, PD-L1, CD47, and/or CD58. In some embodiments, one or more of β2M, TAP1, TAP2, Tapasin, and/or CD58 are modified in an allogeneic immune cell in combination with the overexpression of HLA-E, HLA-G, PD-L1, and/or CD47.

In some embodiments, at least one or more PLC genes (e.g., β2M, TAP1, TAP2, Tapasin, and/or CD58), or regulatory elements thereof, are modified in an allogeneic immune cell in combination with one or more modifications in at least one additional gene sequence or regulatory element thereof and with the overexpression of one or more inhibitory receptors. In some embodiments, one or more of β2M, TAP1, TAP2, and/or Tapasin, are modified in an allogeneic immune cell in combination with modifications in TRAC, CD58, and/or CIITA and with the overexpression of one or more of HLA-E, HLA-G, PD-L1, and/or CD47. In some embodiments, one or more of β2M, TAP1, TAP2, and/or Tapasin are modified in an allogeneic immune cell in combination with one or modifications in TRAC, CD58, and CIITA and with the overexpression of HLA-E, HLA-G, PD-L1, and CD47.

The modified immune cells and methods provided herein address known limitations of CAR-T therapy and is a promising development towards the next generation of precision cell-based therapies.

### Dimeric and Trimeric HLA Constructs

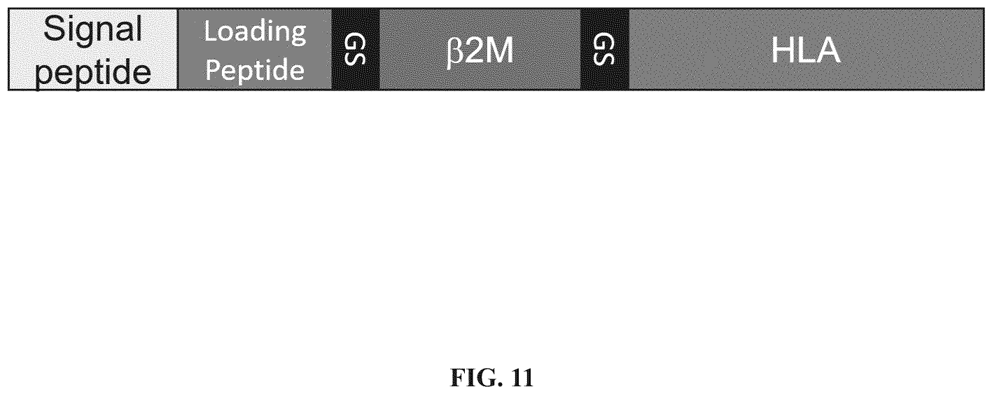

The present disclosure provides human leukocyte antigen (HLA) constructs. The constructs comprise an HLA-E and/or HLA-G domain (e.g., those listed in Table 19 and/or described in Example 10), a signal peptide, and a loading peptide (see FIG. 11). In some embodiments, the HLA construct is membrane-bound or the HLA construct is secreted by a cell. Expression of one or more constructs in a modified immune cell allows for the immune cell to evade NK cells and avoid lysis by NK cells, optionally where the constructs bind to the NKG2A inhibitory receptor of natural killer (NK) cells. In embodiments, a modified immune cell expressing an HLA construct expresses β2M. In some instances, a modified immune cell expressing an HLA construct includes modifications described herein to reduce or eliminate expression of β2M.

The various domains of an HLA construct can be connected by linkers, such as those provided herein. The length of the linkers may be elongated or truncated by about or at least about 1, 2, 3, 4, 5, 6, 7, 8, 9, 10, 15, 20, 25, 30, 35, 40, 45, or 50 amino acids. In various instances, the linker is a Gly/Ser-linker (GS-linker). The length of the linkers may be about, at least about, or no more than about 1, 2, 3, 4, 5, 6, 7, 8, 9, 10, 15, 20, 25, 30, 35, 40, 45, 50, 75, or 100 amino acids.

In some instances, the HLA construct comprises an N-terminal signal peptide (e.g., an IL-2 signal peptide or a β2M signal peptide). Any signal peptide known in the art and suitable for secretion and/or membrane-localization of a polypeptide is suitable in the HLA constructs provided herein.

In some instances the HLA construct contains a transmembrane domain (e.g., any of those transmembrane domains provided herein), optionally at an N-terminal or C-terminal portion thereof. In some embodiments, an HLA construct containing a transmembrane domain further comprises a cytoplasmic portion, where the cytoplasmic portion in various instances is about, at least about, or no more than 1, 2, 3, 4, 5, 10, 15, 20, 25, 30, 35, 40, 45, 50, 75, or 100 amino acids in length. In embodiments, an HLA construct contains any one or more of the domains described in Table 19 and/or in Example 10, fragments thereof, or extensions thereof, where the fragment may correspond to an N-terminal and/or C-terminal truncation by about, at least about, and/or no more than about 1, 2, 3, 4, 5, 6, 7, 8, 9, 10, 15, 20, or 25 amino acids, and where the extension may correspond to an N-terminal and/or C-terminal extension by about, at least about, and/or no more than about 1, 2, 3, 4, 5, 6, 7, 8, 9, 10, 15, 20, or 25 amino acids.

In some embodiments, the HLA construct contains one of the following domain arrangements: Signal peptide-loading peptide-β2M domain-HLA-E/G domain; Signal peptide-β2M domain-loading peptide-HLA-E/G domain; or Signal peptide-loading peptide-HLA-E/G domain-β2M domain. In some cases, any one of these domain arrangements further includes a C-terminal transmembrane domain. In some instances, any one of these domain arrangements can be modified to not include any β2M domain. In some instances, the HLA-E/G domain contains an HLA-G5 intron tail (see Table 19), optionally where the HLA-G5 intron tail is disposed at a C-terminus or C-terminal portion of the HLA-E/G domain.

The transmembrane domain of the constructs provided herein traverse a cell's lipid bilayer cellular membrane. In some embodiments, this domain is derived from a receptor (e.g., an antigen receptor) having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell. Examples of such proteins include, but are not limited to, subunits of the T cell receptor (TCR) complex, PDT, or any of the Cluster of Differentiation proteins, or other proteins, that are expressed in the immune effector cell and that have a transmembrane domain. In some embodiments, the transmembrane domain will be synthetic, and such sequences will comprise many hydrophobic residues.

Transmembrane domains for use in the disclosed HLA constructs can include at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, the transmembrane domain is derived from CD4, CD8α, CD28 and CD3ζ.

### Modified Immune Cells

The present disclosure provides immune cells (e.g., T- or NK-cells) modified using nucleobase editors and/or nucleases described herein. The modified immune cells may express chimeric antigen receptors (CARs) (e.g., CAR-T cells). In embodiments, the modified immune cells express an HLA-E and/or HLA-G single-chain dimer or trimer construct (e.g., those described above and/or listed in Table 19 and/or described in Example 10). Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a diseased cell. Because the CAR-T cells can act independently of major histocompatibility complex (MHC), activated CAR-T cells can kill the diseased cell expressing the antigen. The direct action of the CAR-T cell evades defensive mechanisms that have evolved in response to MHC presentation of antigens to immune cells.

In embodiments, the modified immune cell has a reduced level of, lacks, or has virtually undetectable levels of one or more of the following polypeptides relative to an unmodified cell: B cell leukemia/lymphoma 11b (Bcl11b); B cell leukemia/lymphoma 2 related protein A1d (Bcl2a1d); B cell leukemia/lymphoma 6 (Bcl6); butyrophilin-like 6 (Btnl6); CD151 antigen (Cd151); chemokine (C—C motif) receptor 7 (Ccr7); discs large MAGUK scaffold protein 5 (Dlg5); erythropoietin (Epo); G protein-coupled receptor 18 (Gpr18); interferon alpha 15 (Ifnal5); interleukin 6 signal transducer (Il6st); interleukin 7 receptor (I17r); Janus kinase 3 (Jak3); membrane associated ring-CH-type finger 7 (Marchf7); NCK associated protein 1 like (Nckap11); phospholipase A2, group IIF (Pla2g2f); runt related transcription factor 3 (Runx3); Signal-regulatory protein beta 1B (Sirpb1b); transforming growth factor, beta 1 (Tgfb1); tumor necrosis factor (ligand) superfamily, member 14 (Tnfsf14); tumor necrosis factor (ligand) superfamily, member 18 (Tnfsf18); tumor necrosis factor (ligand) superfamily, member 8 (Tnfsf8); zinc finger CCCH type containing 8 (Zc3h8); (Rac family small GTPase 2); (Slc4a1); 5-azacytidine induced gene 2 (Azi2); a disintegrin and metalloprotease domain 17 (Adam 17); a disintegrin and metalloprotease domain 8 (Adam8); Acetyl-CoA Acetyltransferase 1 (ACAT1); ACLY; adapter related protein complex 3 beta 1 sububit (Ap3b1); adapter related protein complex 3 delta 1 sububit (Ap3d1); adenosine A2a receptor (Adora2a); adenosine deaminase (Ada); adenosine kinase (Adk); adenosine regulating molecule 1 (Adrm1); advanced glycosylation end product-specific receptor (Ager) allograft inflammatory factor 1 (Aif1); AKT1; AKT2; amyloid beta (A4) precursor protein-binding family B member 1 interacting protein (Apbblip); ankyrin repeat and LEM domain (Ankle1); annecin A1 (Anxal); arginase liver (Arg 1); arginase type II (Arg 2); AtPase Cu++ transporting, alpha polypeptide (Atp7a); autoimmune regulator (Aire); autophagy related 5 (Atg5); AXL; B and T Lymphocyte Associated (BTLA); B and T lymphocyte associated (Btla); B cell leukemia/lymphoma 10 (Bcl10); B cell leukemia/lymphoma 11a (Bcl11a); B cell leukemia/lymphoma 2 (Bcl2); B cell leukemia/lymphoma 3 (Bcl3); basic leucine zipper transcription factor, ATF-like (Batf); BCL2-associated X protein (Bax); BCL2L11; beta 2 microglobulin (B2m); BL2-associated agonist of cell dealth (Bad); BLIMP1; Bloom syndrome, RecQ like helicase (Blm); Bmi1 polycomb ring finger oncogene (Bmi1); Bone morphogenic protein 4 (Bmp4); Braf transforming gene (Braf); butyrophilin, subfamily 2, member A1 (Btn2a1); butyrophilin, subfamily 2, member A2 (Btn2a2); butyrophilin-like 1 (Btnl1); butyrophilin-like 2 (Btnl2); c-abl oncogene 1 (Abl1); c-abl oncogene 2 (Abl2); cadherin-like 26(Cdh26); calcium channel, voltage dependent, beta 4 subunit (Cacnb4); CAMK2D; capping protein regulator and myosin 1 linker 2 (Carmil2); carcinoembryonic antigen-related cell adhesion molecule (Ceacam1); Casitas B-lineage lymphoma b (Cblb); CASP8; Caspase 3 (Casp3); caspase recruitment domain family member 11 (Card11); catenin (cadherin associated protein), beta 1 (Ctnnb1); caveolin 1 (Cav1); CBL-B; CCAAT/enhancer binding protein (C/EBP), beta (Cebpb); CCR10; CCR4; CCR5; CCR6; CCR9; CD103; CD11a; CD122; CD123; CD127; CD130; CD132; CD160 antigen (Cd160); CD161; CD19; CD1d1 antigen (Cd1d1); CD1d2 antigen (CD1d2); CD2 antigen (CD2); CD209e antigen (Cd209e); CD23; CD244 molecule A (Cd244a); CD24a antigen (Cd24a); CD27 antigen (CD27); CD274 antigen (Cd274); CD276 antigen (Cd276); CD28 antigen (Cd28); CD3 delta; CD3 epsilon; CD3 gamma; CD30; CD300A molecule (Cd300a); CD33; CD38; CD4 antigen (Cd4); CD40 ligand (Cd40lg); CD44 antigen (Cd44); CD46 antigen, complement regulatory protein (Cd46); CD47 antigen (Rh-related antigen, integrin-associated signal transducer) (Cd47); CD48 antigen (Cd48); CD5 antigen (Cd5); CD52; CD58; CD59b antigen (Cd59b); CD6 antigen (Cd6); CD69; CD7; CD70; CD74 antigen (Cd74); CD8; CD8 antigen (Cd8); CD80 antigen (Cd80); CD81 antigen (Cd81); CD82; CD83 antigen (Cd83); CD86; CD86 antigen (Cd86); CD8A; CD96; CD99; CDK4; CDK8; CDKN1B; chemokine (C motif) ligand 1 (Xcl1); chemokine (C—C motif) ligand 19 (Ccl19); chemokine (C—C motif) ligand 2 (Ccl2); chemokine (C—C motif) ligand 20 (Ccl20); chemokine (C—C motif) ligand 5 (Ccl5); chemokine (C—C motif) receptor 2 (Ccr2); chemokine (C—C motif) receptor 6 (Ccr6); chemokine (C—C motif) receptor 9 (Ccr9); chemokine (C—X—C motif) ligand 12 (Cxcl12); chemokine (C—X—C motif) receptor (Cxcr4); Chitinase 3 Like 1 (Chi311); cholinergic receptor, nicotinic, alpha polypeptide 7 (Chrna7); chromodomain helicase DNA binding protein 7 (Chd7); CLA; Class II Major Histocompatibility Complex Transactivator (CIITA); cleft lip and palate associated transmembrane protein 1 (Clptm1); Cluster of Differentiation 123 (CD123); Cluster of Differentiation 3 (CD3); Cluster of Differentiation 33 (CD33); Cluster of Differentiation 52 (CD52); Cluster of Differentiation 7 (CD7); Cluster of Differentiation 96 (CD96); coagulation factor II (thrombin) receptor-like 1 (F2rl1); coil-coil domain containing 88B (Ccdc88b); core-binding factor beta (Cbfb); coronin, actin binding protein 1A (Coro1a); coxsackie virus and adenovirus receptor (Cxadr); CS-1; CSF2CSK; c-src tyrosine kinase (Csk); C-type lectin domain family 2, member i (Clec2i); C-type lectin domain family 4, member a2 (Clec4a2); C-type lectin domain family 4, member d (Clec4d); C-type lectin domain family 4, member e (Clec4e); C-type lectin domain family 4, member f (Clec4f); C-type lectin domain family 4, member g (Clec4g); CUL3; CXCR3; cyclic GMP-AMP synthase (Cgas); cyclin D3 (Ccnd3); cyclin dependent kinase inhibitor 2A (Cdkn2a); cyclin-dependent kinase (Cdk6); CYLD lysine 63 deubiquitinase (Cyld); cysteine-rich protein 3 (Crip3); cytidine 5′-triphosphate synthase (Ctps); Cytochrome P450 Family 11 Subfamily A Member 1 (Cyp11a1); cytochrome P450, family 26, subfamily b, polypeptide (Cyp26b1); Cytokine Inducible SH2 Containing Protein (CISH); cytotoxic T lymphocyte-associated protein 2 alpha (Ctla2a); Cytotoxic T-Lymphocyte Associated Protein 4 (CTLA-4); DCK; dedicator of cytokinesis 2 (Dock2); dedicator of cytokinesis 8 (Dock8); delta like canonical Notch ligand 4 (D114); deltex 1, E3 ubiquitin ligase (Dtx1); deoxyhypusine synthase (Dhps); DGKA; DGKZ; DHX37; dicer 1, ribonuclease type III (Dicer1); dipeptidylpeptidase 4 (Dpp4); discs large MAGUK scaffold protein 1 (Dlg1); DnaJ heat shock protein family (Hsp40) member A3 (Dnaja3); dolichyl-di-phosphooligosaccharide-protein glycotransferase (Ddost); double homeobox B-like 1 (Duxbl1); drosha, ribonuclease type III (Drosha); dual specificity phosphatase 10 (Dusp10); dual specificity phosphatase 22 (Dusp22); dual specificity phosphatase 3 (Dusp3); E74-like factor 4 (Elf4); early growth response 1 (Egr1); early growth response 3 (Egr3); ELOB (TCEB2); ENTPD1 (CD39); eomesodermin (Eomes); Eph receptor B4 (Ephb4); Eph receptor B6 (Ephb6); ephrin B1 (Efnbl); ephrin B2 (Efnb2); ephrin B3 (Efnb3); Epstein-Barr virus induced gene 3 (Ebi3); erb-b2 receptor tyrosine kinase (Erbb2); eukaryotic translation initiation factor 2 alpha kinase 4 (Eif2ak4); FADD; family with sequence similarity 49, member B (Fam49b); Fanconi anemia, complementation group A (Fanca); Fanconi anemia, complementation group D2 (Fancd2); Fas (TNF receptor superfamily member 6) (Fas); Fas (TNFRSF6)-associated via death domain (Fadd); Fas Cell Surface Death Receptor (FAS); Fc receptor, IgE, high affinity I, gamma polypeptide (Fcer1g); fibrinogen-like protein 1 (Fgl1); fibrinogen-like protein 2 (Fgl2); FK506 binding protein 1a (Fkbp1a); FK506 binding protein 1b ((Fkbp1b); flotillin 2 (Flot2); FMS-like tyrosine kinase (Flt3); forkhead box J1 (Foxj1); forkhead box N1 (Foxn1); forkhead box P1 (Foxp1); forkhead box P3 (Foxp3); frizzled class receptor 5 (Fzd5); frizzled class receptor 7 (Fzd7); frizzled class receptor 8 (Fzd8); fucosyltransferase 7 (Fut7); Fyn proto-oncogene (Fyn); gap junction protein, alpha 1 (Gja1); GATA binding protein 3 (GATA3); GCN2 kinase (IDO pathway); gelsolin (Gsn); GLI-Kruppel family member GLI3 (Gli3); glycerol-3-phosphate acyltransferase, mitochondrial (Gpam); growth arrest and DNA-damage-inducible 45 gamma (Gadd45g); GTPase, IMAP family member 1 (Gimap1); H1TET2; H2.0-like homeobox (Hlx); haematopoietic T (hem1); HCLS1 binding protein 3 (Hs1bp3); heat shock 105 kDa/110 kDa protein 1 (Hsph1); heat shock protein 1 (chaperonin) (Hspd1); heat shock protein 90, alpha (cytosolic), class A member 1 (Hsp90aa1); hematopoietic SH2 domain containing (Hsh2d); hepatitis A virus cellular receptor 2 (Havcr2); hes family bHLH transcription factor 1 (Hes1); histocompatibility 2, class II antigen A, alpha (H2-Aa); histocompatibility 2, class II antigen A, beta 1 (H2-Abl); histocompatibility 2, class II, locus DMa (H2-DMa); histocompatibility 2, M region locus 3 (H3-M3); histocompatibility 2, O region alpha locus (H2-Oa); histocompatibility 2, T region locus 23 (H2-T23); HLA-DR; homeostatic iron regulator (Hfe); icos ligand (Icosl); IKAROS family zinc finger 1 (Ikzf1); IL10; IL10RA; IL2 inducible T cell kinase (Itk); IL6R; Indian hedgehog (Ihh); indoleamine 2,3-dioxygenase 1 (Idol); inducible T cell co-stimulator (Icos); inositol 1,4,5-trisphosphate 3-kinase B (Itpkb); insulin II (Ins2); insulin-like growth factor 1 (Igf1); insulin-like growth factor 2 (Igf2); insulin-like growth factor binding protein 2 (Igfbp2); integrin alpha L (Itgal); integrin alpha M (Itgam); integrin alpha V (Itgav); integrin alpha X (Itgax); integrin beta 2 (Itgb2); integrin, alpha D (Itgad); intercellular adhesion molecule 1 (Icam1); interferon (alpha and beta) receptor 1 (Ifnar1); interferon alpha 1 (Ifna1); interferon alpha 11 (Ifnal1); interferon alpha 12 (Ifnal2); interferon alpha 13 (Ifnal3); interferon alpha 14 (Ifna14); interferon alpha 16 (Ifnal6); interferon alpha 2 (Ifna2); interferon alpha 4 (Ifna4); interferon alpha 5 (Ifna5); interferon alpha 6 (Ifna6); interferon alpha 7 (Ifna7); interferon alpha 9 (Ifna9); interferon alpha B (Ifnab); interferon beta 1 (Ifnb1); interferon gamma (Ifng); interferon kappa (Ifnk); interferon regulatory factor 1 (Irf1); interferon regulatory factor 4 (Irf4); interferon zeta (Ifnz); interleukin 1 beta (Il1b; interleukin 1 family, member 8 (Illf8); interleukin 1 receptor-like 2 (Il1r12); interleukin 12 receptor, beta1 (Il12rb1); interleukin 12a (Il12a); interleukin 12b (Il12b); interleukin 15 (I115); interleukin 18 (Il18); interleukin 18 receptor 1 (Il18r1); interleukin 2 (I12); interleukin 2 receptor, alpha chain (Il2ra); interleukin 2 receptor, gamma chain (Il2rg); interleukin 20 receptor beta (Il20rb); interleukin 21 (Il21); interleukin 23, alpha subunit p19 (I123a); interleukin 27 (I127); interleukin 4 (I14); interleukin 4 receptor, alpha (Il4ra); interleukin 6 (I16); interleukin 7 (I17); IRF8; itchy, E3 ubiquitin protein ligase (Itch); jagged 2 (Jag2); jumonji domain containing 6 (Jmjd6); JUNB; junction adhesion molecule like 9 (Jam9); K(lysine) acetyltransferase 2A (Kat2a); KDEL (Lys-Asp-Glu-Leu) endoplasmic reticulum protein retention receptor 1 (Kdelr1); KIT proto-oncogene receptor tyrosine kinase (Kit); LAG-3; LAIR-1 (CD305); LDHA; lectin, galactose binding, soluble 1 (Lgals1); lectin, galactose binding, soluble 3 (Lgals3); lectin, galactose binding, soluble 8 (Lgals8); lectin, galactose binding, soluble 9 (Lgals9); leptin (Lep); leptin receptor (Lepr); leucine rich repeat containing 32 (Lrrc32); leukocyte immunoglobulin-like receptor, subfamily B, member 4A (Lilrb4a); LFNG O-fucosylpeptide 3-beta-N-acetylglucosaminyltransferase (Lfng); LIF; ligase IV, DNA, ATP-dependent (Lig4); LIM domain only 1 (Lmo1); limb region 1 like (Lmbrl); linker for activation of T cells (Lat); lymphocyte antigen 9 (Ly9); lymphocyte cytosolic protein 1 (Lcp1); lymphocyte protein tyrosine kinase (Lck); lymphocyte transmembrane adaptor 1 (Lax1); lymphocyte-activation gene 3 (Lag3); lymphoid enhancer binding factor 1 (Lef1); LYN; lysyl oxidase-like 3 (Loxl3); MAD1 mitotic arrest deficient 1-like 1 (Mad1l1); MALT1 paracaspase (Malt1); MAP4K4; MAPK14; MCJ; mechanistic target of rapamycin kinase (Mtor); MEF2D; Methylation-Controlled J Protein (MCJ); methyltransferase like 3 (Mettl3); MGAT5; MHC I like leukocyte 2 (Mill2); midkine (Mdk); mitogen-activated protein kinase 8 interacting protein 1 (Mapk8ip10); moesin (Msn); myelin protein zero-like 2 (Mpzl2); myeloblastosis oncogene (Myb); myosin, heavy polypeptide 9, non-muscle (Myh9); Nedd4 family interacting protein 1 (Ndfip1); neural precursor cell expressed, developmentally down-regulated 4 (Nedd4); NFATc1; NFATC2; NFATC4; NFKB activating protein (Nkap); nicastrin (Ncstn); NK2 homeobox 3 (Nkx2-3); NLR family, CARD domain containing 3 (Nlrc3); NLR family, pyrin domain containing 3 (Nlrp3); non-catalytic region of tyrosine kinase adaptor protein 1 (Nck1); non-catalytic region of tyrosine kinase adaptor protein 2 (Nck2); non-homologous end joining factor 1 (Nhej1); non-SMC condensin II complex, subunit H2 (Ncaph2); Notch-regulated ankyrin repeat protein (Nrarp); NT5E (CD73); nuclear factor of activated T cells, cytoplasmic, calcineurin dependent (Nfatc3); nuclear factor of kappa light polypeptide gene enhancer in B cells inhibitor, delta (Nfkbid); nuclear receptor co-repressor 1 (Ncor1); Nuclear Receptor Subfamily 4 Group A Member 1 (NR4A1); Nuclear Receptor Subfamily 4 Group A Member 2 (NR4A2); Nuclear Receptor Subfamily 4 Group A Member 3 (NR4A3); ODC1; OTU domain containing 5 (Otud5); OTULINL (FAM105A); paired box 1 (Pax1); PDCD1 (PD1; PD-1); PDIA3; pellino 1 (Peli1); peroxiredoxin 2 (Prdx2); PHD1 (EGLN2); PHD2 (EGLN1); PHD3 (EGLN3); phosphodiesterase 5A, cGMP-specific (Pde5a); phosphoinositide-3-kinase regulatory subunit (Pik3r6); phospholipase A2, group IIA (Pla2g2a); phospholipase A2, group IID (Pla2g2d); phospholipase A2, group IIE (Pla2g2e); phosphoprotein associated with glycosphingolipid microdomains 1 (Pag1); PIK3CD; PIKFYVE; POZ (BTB) and AT hook containing zinc finger 1 (Patz1); PPARa; PPARd; PR domain containing 1, with ZNF domain (Prdm1); presenilin 1 (Psen1); presenilin 2 (Psen2); PRKACA; PRKC, apoptosis, WT1, regulator (Pawr); programmed cell death 1 ligand 2 (Pdcd1lg2); prosaposin (Psap); prostaglandin E receptor 4 (subtype EP4) (Ptger4); protein kinase C, theta 2 (Prkcq); protein kinase C, zeta (Prkcz); protein kinase, cAMP dependent regulatory, type I, alpha (Prkar1a); protein kinase, DNA activated, catalytic polypeptide (Prkdc); protein phosphatase 3, catalytic subunit, beta isoform (Ppp3cb); protein tyrosine phosphatase, non-receptor type 2 (Ptpn2); protein tyrosine phosphatase, non-receptor type 22 (lymphoid) (Ptpn22); protein tyrosine phosphatase, non-receptor type 6 (Ptpn6); protein tyrosine phosphatase, receptor type, C (Ptprc); PTEN; PTPN11; purine-nucleoside phosphorylase (Pnp); purinergic receptor P2X, ligand-gated ion channel, 7 (P2rx7); PVR Related Immunoglobulin Domain Containing (PVRIG; CD112R); PYD and CARD domain containing 7 (Pycard); RAB27A, member RAS oncogene family (Rab27a); RAB29, member RAS oncogene family (Rab29); radical S-adenosyl methionine domain containing 2 (Rsad2); RAR-related orphan receptor alpha (Rora); RAR-related orphan receptor gamma (Ror); RAS guanyl releasing protein 1 (Rasgrpl); ras homolog family member A (Rhoa); ras homolog family member H (Rhoh); RAS protein activator like 3 (Rasal3); RASA2; receptor (TNFRSF)-interacting serine-threonine kinase 2 (Ripk2); recombination activating gene 1 (Rag1); recombination activating gene 2 (Rag2); Regulatory Factor X Associated Ankyrin Containing Protein (RFXANK); RHO family interacting cell polarization regulator 2 (Ripor2); ribosomal protein L22 (Rpl 22); ribosomal protein S6 (Rps6); RING CCCH (C3H) domains 1 (Rc3h1); ring finger and CCCH-type zinc finger domains 2 (Rc3h2); RNF2; runt related transcription factor 1 (Runx1); runt related transcription factor 2 (Runx2); SAM and SH3 domain containing 3 (Sash3); schlafen 1; Selectin P Ligand/P-Selectin Glycoprotein Ligand-1 (SELPG/PSGL1) polypeptide; selenoprotein K (Selenok); sema domain immunoglobulin domain (Ig), transmembrane domain (TM) and short cytoplasmic domain, (semaphorin) 4A (Sema4a); serine/threonine kinase 11 (Stk11); SH3 domain containing ring finger 1 (Sh3rf1); SHP1; sialophorin (Spn); SIGLEC15; signal transducer and activator of transcription 3 (Stat3); signal transducer and activator of transcription 5A (Stat5A); signal transducer and activator of transcription 5B (Stat5B); signal-regulatory protein alpha (Sirpa); Signal-regulatory protein beta 1A (Sirpb1a); Signal-regulatory protein beta 1C (Sirpb1c); SLA; SLAM family member 6 (Slamf6); SLAMF7; SMAD family member 3 (Smad3); SMAD family member 7 (Smad7); SMARCA4; solute carrier family 11 (proton-coupled divalent metal ion transporters), member 1 (Slc11 al); solute carrier family 4 (anion exchanger), member 1; solute carrier family 46, member 2 (Slc46a2); sonic hedgehog (Shh); SOS Ras/Rac guanine nucleotide exchange factor 1 (Sos1); SOS Ras/Rac guanine nucleotide exchange factor 2 (Sos2); special AT-rich sequence binding protein 1 (Satb1); spleen tyrosine kinase (Syk); Sprouty RTK Signaling Antagonist 1 (Spry1); Sprouty RTK Signaling Antagonist 2 (Spry2); squamous cell carcinoma antigen recognized by T cells (Sart1); src homology 2 domain-containing transforming protein B (Shb); Src-like-adaptor 2 (Sla2); SRY (sex determining region Y)-box 4 (Sox4); STK4; suppression inducing transmembrane adaptor 1 (Sit1); suppressor of cytokine signaling 1 (Socs1); suppressor of cytokine signaling 5 (Socs5); suppressor of cytokine signaling 6 (Socs6); surfactant associated protein D (Sftpd); SUV39; syndecan 4 (Sdc4); syntaxin 11 (Stx11); T Cell Immunoglobulin Mucin 3 (Tim-3); T cell immunoreceptor with Ig and ITIM domains (Tigit); T cell receptor alpha joining 18 (Traj18); T Cell Receptor Beta Constant 1 (TRBC1); T Cell Receptor Beta Constant 2 (TRBC2); T cell, immune regulator 1, ATPase, H+ transporting, lysosomal V0 protein A3 (Tcirg1); T cell-interacting, activating receptor on myeloid cells 1 (Tarm1); T-box 21 (Tbx21); TCR; TCR alpha; TCR beta; TCR complex gene sequence; Tet Methylcytosine Dioxygenase 2 (TET2); TGFbRII; TGFbRII (TGFBR2); three prime repair exonuclease 1 (Trex1); thymocyte selection associated (Themis); thymus cell antigen 1, theta (Thy 1); TMEM222; TNF receptor-associated factor 6 (Traf6); TNFAIP3; TNFRSF10B; TNFRSF8 (CD30); TOX; TOX2; TRAC; transformation related protein 53 (Trp53); Transforming Growth Factor Beta Receptor II (TGFbRII); transforming growth factor, beta receptor II (Tgfbr2); transmembrane 131 like (Tmeml311); transmembrane protein 98 (Tmem98); triggering receptor expressed on myeloid cells-like 2 (Treml2); TSC complex subunit 1 (Tsc1); tumor necrosis factor (ligand) superfamily, member 11 (Tnfsf11); tumor necrosis factor (ligand) superfamily, member 13b (Tnfsf13b); tumor necrosis factor (ligand) superfamily, member 4 (Tnfsf4); tumor necrosis factor (ligand) superfamily, member 9 (Tnfsf9); tumor necrosis factor receptor superfamily, member 13c (Tnfrsf13c); tumor necrosis factor receptor superfamily, member 4 (Tnfrsf4); tumor necrosis factor, alpha-induced protein 8-like 2 (Tnfalp812); twisted gastrulation BMP signaling modulator 1 (Twsg1); UBASH3A; vanin 1 (Vnn1); vascular cell adhesion molecule 1 (Vcam1); VHL; v-maf musculoaponeurotic fibrosarcoma oncogene family, protein B (avian) (Mafb); V-set and immunoglobulin domain containing 4 (Vsig4); V-Set Immunoregulatory Receptor (VISTA); WD repeat and FYVE domain containing 4 (Wdfy4); wingless-type MMTV integration site family, member 1 (Wnt1); wingless-type MMTV integration site family, member 4 (Wnt4); WNT signaling pathway regulator (Apc); WW domain containing E3 ubiquitin protein ligase 1 (Wwp1); XBP1; YAP1; ZAP70; ZC3H12A; zfp35; zinc finger and BTB domain containing 1 (Zbtb1); zinc finger and BTB domain containing 7B (Zbtb7B); zinc finger CCCH type containing 12A (Zc3h12a); zinc finger CCCH type containing 12D (Zc3h12d); zinc finger E-box binding homeobox 1 (Zeb1); zinc finger protein 36, C3H type (Zfp36); zinc finger protein 36, C3H type-like 1 (Zfp36L1); zinc finger protein 36, C3H type-like 2 (Zfp36L2); and zinc finger protein 683 (Zfp683).

Some embodiments comprise allogeneic immune cell immunotherapy. In allogeneic immune cell immunotherapy, immune cells are obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor (CAR), are administered to a subject for treating a disease. In some embodiments, immune cells to be modified to express a chimeric antigen receptor (CAR) can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

In another embodiment, the present disclosure provides T cells that have targeted gene knock-outs at the TCR constant region (TRAC), which is responsible for TCRαβ surface expression. TCRαβ-deficient CAR-T cells are compatible with allogeneic immunotherapy (Qasim et al., Sci. Transl. Med. 9, eaaj2013 (2017); Valton et al., Mol Ther. 2015 September; 23(9): 1507-1518). If desired, residual TCRαβ T cells are removed using CliniMACS magnetic bead depletion to minimize the risk of GVHD. In another embodiment, the present disclosure provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115(19):3869-3878).

Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

The immune effector cells contemplated in the present disclosure are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, immune effector cells are effector NK cells. In some embodiments, the immune effector cell is any other subset of T cells. The modified immune effector cell may express, in addition to the chimeric antigen receptor (CAR), an exogenous cytokine, a different chimeric receptor, or any other agent that would enhance immune effector cell signaling or function. For example, co-expression of the chimeric antigen receptor and a cytokine may enhance the CAR-T cell's ability to lyse a target cell.

Chimeric antigen receptors (CARs) as contemplated in the present disclosure may comprise an extracellular binding domain, a transmembrane domain, and an intracellular domain. Binding of an antigen to the extracellular binding domain can activate the CAR-T cell and generate an effector response, which includes CAR-T cell proliferation, cytokine production, and other processes that lead to the death of the antigen expressing cell. Exemplary CARs include those described in Maldini, et al. “Dual CD4-based CAR-T cells with distinct costimulatory domains mitigate HIV pathogenesis in vivo,” Nat. Med. 26:1776-1787 (2020); Maldini, et al. “HIV-Resistant and HIV-Specific CAR-Modified CD4+ T Cells Mitigate HIV Disease Progression and Confer CD4+ T Cell Help In Vivo,” Mol. Ther. 28:1585-1599 (2020); and Leibman, et al. “Supraphysiologic control over HIV-1 replication mediated by CD8 T cells expressing a re-engineered CD4-based chimeric antigen receptor,” PLoS Pathog. 13(10):e1006613(2017), the entire disclosure of each of which is incorporated herein by reference in its entirety for all purposes.

In some embodiments of the present disclosure, the chimeric antigen receptor further comprises a linker. In some embodiments, the linker is a (GGGGS)ₙ linker (SEQ ID NO: 247). In some embodiments, the linker is a (GGGGS)₃ linker (SEQ ID NO: 478). In some embodiments, a CAR of the present disclosure includes a leader peptide sequence (e.g., N-terminal to the antigen binding domain). An exemplary leader peptide amino acid sequence is:

(SEQ ID NO: 479)
METDTLLLWVLLLWVPGSTG.

Provided herein are also nucleic acids that encode the chimeric antigen receptors (CARs) described herein. In some embodiments, the nucleic acid molecule is isolated or purified. Delivery of the nucleic acid molecules ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present disclosure provide for immune cells comprising a chimeric antigen receptor (CAR) and an altered endogenous gene that provides increased persistence, resistance to fratricide, enhances immune cell function, resistance to immunosuppression or inhibition, or a combination thereof. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in an exon, an intron, an exon-intron injunction, or a regulatory element thereof. The modification may be edit to a single nucleobase in a gene or a regulatory element thereof. The modification may be in a exon, more than one exons, an intron, or more than one introns, or a combination thereof. The modification may be in an open reading frame of a gene. The modification may be in an untranslated region of the gene, for example, a 3′-UTR or a 5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g. a Kozak sequence), or any combination thereof.

Allogeneic immune cells expressing an endogenous immune cell receptor, as well as a chimeric antigen receptor (CAR) may recognize and attack host cells, a circumstance termed graft versus host disease (GVHD). The alpha component of the immune cell receptor complex is encoded by the TRAC gene, and in some embodiments, this gene is edited such that the alpha subunit of the TCR complex is nonfunctional or absent. Because this subunit is necessary for endogenous immune cell signaling, editing this gene can reduce the risk of graft versus host disease caused by allogeneic immune cells.

In some embodiments, editing of genes to provide increased persistence, fratricide resistance, enhance the function of the immune cell or to reduce immunosuppression or inhibition can occur in the immune cell before the cell is transformed to express a chimeric antigen receptor (CAR). In other aspects, editing of genes to increase persistence, provide fratricide resistance, enhance the function of the immune cell or to reduce immunosuppression or inhibition can occur in a CAR-T cell, i.e., after the immune cell has been transformed to express a chimeric antigen receptor (CAR).

In some embodiments, the immune cell may comprise one or more edited genes, one or more regulatory elements thereof, or combinations thereof, wherein expression of the edited gene is either knocked out or knocked down. In some embodiments, the immune cell may comprise one or more edited genes, one or more regulatory elements thereof, or combinations thereof, wherein expression of the edited gene is increased. In some embodiments, the immune cell may comprise a chimeric antigen receptor (CAR) and one or more edited genes, one or more regulatory elements thereof, or combinations thereof, wherein expression of the edited gene is either knocked out or knocked down. In some embodiments, the immune cell may comprise a chimeric antigen receptor (CAR) and one or more edited genes, one or more regulatory elements thereof, or combinations thereof, wherein expression of the edited gene is increased.

In some embodiments, the CAR-T cells have reduced (e.g., a negative alteration of at least 10%, 25%, 50%, 75%, or 100%) or inactivated surface HLA class-I expression as compared to a similar CAR-T cell lacking one or more edited genes as described herein. In some embodiments, the CAR-T cells have increased persistence as compared to a similar CAR-T cell lacking one or more edited genes as described herein. In some embodiments, the CAR-T cells have increased fratricide resistance as compared to a similar CAR-T cell lacking one or more edited genes as described herein. In some embodiments, the CAR-T cells have reduced immunogenicity as compared to a similar CAR-T cell lacking one or more edited genes as described herein. In some embodiments, the CAR-T cells have lower activation threshold as compared to a similar CAR-T lacking one or more edited genes as described herein. In some embodiments, the CAR-T cells have increased anti-neoplasia activity as compared to a similar CAR-T cell lacking one or more edited genes as described herein. In some embodiments, the CAR-T cells have increased T- and/or NK-cell resistance as compared to a similar CAR-T cell lacking one or more edited genes as described herein. The one or more genes may be edited by base editing. In some embodiments the one or more genes are directed to components of the peptide loading complex (PLC) or regulatory components thereof. In some embodiments the one or more genes may be selected from a group consisting of: β2M, TAP1, TAP2, Tapasin, and CD58. In some the genes may be edited by base editing and or using a nuclease (e.g., Cas12b). In some embodiments, the one or more genes are selected from CD58, CD115, CD48, MICA, MICB, Nectin-2, ULBP, β2M, TAP1, TAP2, TAPBP, PDIA3, NLRC5, HLA-A, HLA-B, and/or HLA-C. In some embodiments, one or more additional genes may be edited using a base editor or nuclease. In some embodiments, the one or more additional genes may be selected from TRAC, and CIITA. In some embodiments, the one or more additional genes edited may be selected from HLA-E, HLA-G, PD-L1, and CD47. In some embodiments, one or more of β2M, TAP1, TAP2, Tapasin, and/or CD58 are edited in combination with edits in each of HLA-E, HLA-G, PD-L1, and CD47. In some embodiments, a β2M gene is not edited. In some embodiments, the CAR-T cells are base-edited to have reduced or eliminated HLA-A and HLA-B expression but unmodified HLA-C expression, and a β2M gene is not edited. In some embodiments, the CAR-T cells are base-edited to have reduced or eliminated HLA-A and HLA-B expression but unmodified HLA-C expression, and a β2M gene is not edited, and additionally, at least one additional gene is edited.

In some embodiments, an immune cell comprises a chimeric antigen receptor and one or more edited genes, a regulatory element thereof, or combinations thereof. An edited gene may be an immune response regulation gene, an immunogenic gene, a checkpoint inhibitor gene, a gene involved in immune responses, a cell surface marker, e.g. a T cell surface marker, or any combination thereof. In some embodiments, an immune cell comprises a chimeric antigen receptor and an edited gene that is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene, for example, such as a PD1 gene, a PDC1 gene, or a member related to or regulating the pathway of their formation or activation.

In some embodiments, provided herein is an immune cell with an edited gene in the peptide loading complex (PLC) or a regulatory element thereof, such that the immune cell does not express or expresses at reduced levels surface HLA class-I peptides. In some embodiments, provided herein is an immune cell with an edited gene in the peptide loading complex (PLC) or a regulatory element thereof, such that the immune cell has increased persistence. In some embodiments, the immune cell comprises an edited gene in the peptide loading complex (PLC) or a regulatory element thereof, and additionally, at least one edited gene.

In some embodiments, provided herein is an immune cell (e.g., T- or NK-cell) with an edited β2M gene, such that the immune cell does not express an endogenous functional Beta-2-microglobulin. In some embodiments, provided herein is an immune cell with an edited β2M gene, such that the immune cell does not express or expresses at reduced levels surface HLA class-I peptides. In some embodiments, provided herein is an immune cell with an edited β2M gene, such that the immune cell has increased persistence. In some embodiments, the immune cell comprises an edited β2M gene, and additionally, at least one edited gene.

In some embodiments, provided herein is an immune cell (e.g., T- or NK-cell) with an edited TAP1 gene, such that the immune cell does not express an endogenous functional TAP1. In some embodiments, provided herein is an immune cell with an edited TAP1 gene, such that the immune cell does not express or expresses at reduced levels surface HLA class-I peptides. In some embodiments, provided herein is an immune cell with an edited TAP1 gene, such that the immune cell has increased persistence. In some embodiments, the immune cell comprises an edited TAP1 gene, and additionally, at least one edited gene.

In some embodiments, provided herein is an immune cell (e.g., T- or NK-cell) with an edited TAP2 gene, such that the immune cell does not express an endogenous functional TAP2. In some embodiments, provided herein is an immune cell with an edited TAP2 gene, such that the immune cell does not express or expresses at reduced levels surface HLA class-I peptides. In some embodiments, provided herein is an immune cell with an edited TAP2 gene, such that the immune cell has increased persistence. In some embodiments, the immune cell comprises an edited TAP2 gene, and additionally, at least one edited gene.

In some embodiments, provided herein is an immune cell (e.g., T- or NK-cell) with edited TAP1 and TAP2 genes, such that the immune cell does not express endogenous functional TAP1 and TAP2. In some embodiments, provided herein is an immune cell with edited TAP1 and TAP2 genes, such that the immune cell does not express or expresses at reduced levels surface HLA class-I peptides. In some embodiments, provided herein is an immune cell with an edited TAP1 and TAP2 gene, such that the immune cell has increased persistence. In some embodiments, the immune cell comprises an edited TAP1 and TAP2 gene, and additionally, at least one edited gene.

In some embodiments, provided herein is an immune cell (e.g., T- or NK-cell) with an edited Tapasin gene, such that the immune cell does not express an endogenous functional Tapasin. In some embodiments, provided herein is an immune cell with an edited Tapasin gene, such that the immune cell does not express or expresses at reduced levels surface HLA class-I peptides. In some embodiments, provided herein is an immune cell with an edited Tapasin gene, such that the immune cell has increased persistence. In some embodiments, the immune cell comprises an edited Tapasin gene, and additionally, at least one edited gene.

In some embodiments, provided herein is an immune cell (e.g., T- or NK-cell) with an edited CD58 gene, such that the immune cell does not express an endogenous functional CD58. In some embodiments, provided herein is an immune cell with an edited CD58 gene, such that the immune cell has increased persistence. In some embodiments, the immune cell comprises an edited CD58 gene, and additionally, at least one edited gene.

In some embodiments, each edited gene may comprise a single base edit. In some embodiments, each edited gene may comprise multiple base edits at different regions of the gene. In some embodiments, a single modification event (such as electroporation), may introduce one or more gene edits. In some embodiments at least one, two, three, four, five, six, seven, eight, nine, ten, eleven, twelve, thirteen, fourteen, fifteen, sixteen, seventeen, eighteen, nineteen, twenty or more edits may be introduced in one or more genes simultaneously. In some embodiments, an immune cell, including but not limited to any immune cell comprising an edited gene selected from any of the aforementioned gene edits, can be edited to generate mutations in other genes that enhance the CAR-T's function or reduce immunosuppression or inhibition of the cell.

### Editing of Target Polynucleotides in Immune Cells

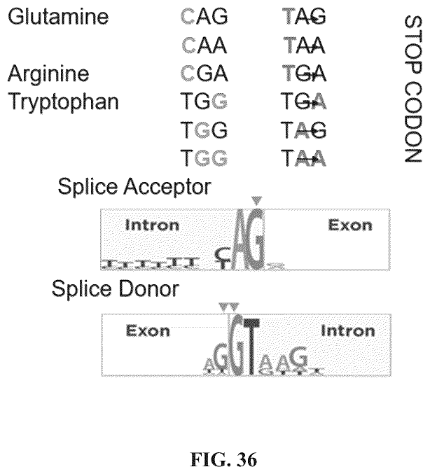

In general, base editing is carried out to induce therapeutic changes in the genome of a cell (e.g., immune cell (e.g., T- or NK-cell)). Base editing can be carried out in vitro or in vivo. In various embodiments, base editing can be used to introduce a stop codon to a gene, to disrupt a splice motif (e.g., a acceptor site, or a splice donor site) (see FIG. 36). In some embodiments, cells (e.g., immune cell (e.g., T- or NK-cell)) are collected from a subject or a donor. In some embodiments, base editing is carried out to induce therapeutic changes in the genome of an immune cell (e.g., T- or NK-cell). In some embodiments, base editing is carried out to induce therapeutic changes in the genome of an allogeneic immune cell (e.g., T- or NK-cell) of a subject. In some embodiments, base editing is carried out to induce therapeutic changes in the genome of an allogeneic CAR-T cell.

To produce the gene edits described above, immune cells are collected from a subject and contacted with one or more guide RNAs and a nucleobase editor polypeptide comprising a nucleic acid programmable DNA binding protein (napDNAbp) and a cytidine deaminase or adenosine deaminase, or comprising one or more deaminases with cytidine deaminase and/or adenosine deaminase activity (e.g., a “dual deaminase” which has cytidine and adenosine deaminase activity). In some embodiments, cells to be edited are contacted with at least one nucleic acid, wherein the at least one nucleic acid encodes one or more guide RNAs and a nucleobase editor polypeptide comprising a nucleic acid programmable DNA binding protein (napDNAbp) and a cytidine deaminase. In some embodiments, the gRNA comprises nucleotide analogs. In some instances, the gRNA is added directly to a cell. These nucleotide analogs can inhibit degradation of the gRNA from cellular processes. Tables 1A-1E and SEQ ID NOs: 1214-2908, 403-412, and 435-446 provide representative target sequences and spacer sequences to be used for gRNAs, as well as representative gRNA sequences.

In some embodiments, immune cells (e.g., T- or NK-cell) of the present disclosure, are contacted with one or more guide RNAs and a nucleobase editor polypeptide comprising a nucleic acid programmable DNA binding protein (napDNAbp) (e.g., Cas9) domain and a deaminase (e.g., cytidine deaminase and/or adenosine deaminase) domain. In some embodiments, immune cells (e.g., T- or NK-cell) of the present disclosure, are contacted with one or more guide RNAs and a nucleobase editor polypeptide comprising a nucleic acid programmable DNA binding protein (napDNAbp) (e.g., Cas9) domain and an adenosine deaminase domain. In some embodiments, immune cells (e.g., T- or NK-cell) of the present disclosure, are contacted with one or more guide RNAs and a nucleobase editor polypeptide comprising a nucleic acid programmable DNA binding protein (napDNAbp) (e.g., Cas9) domain and a cytidine deaminase domain. In some embodiments, immune cells (e.g., T- or NK-cell) of the present disclosure, are contacted with one or more guide RNAs and a nucleobase editor polypeptide comprising a nucleic acid programmable DNA binding protein (napDNAbp) (e.g., Cas9) domain and an adenosine/cytidine deaminase domain. In some embodiments, the at least one nucleic acid molecule encoding one or more guide RNAs and a nucleobase editor polypeptide is delivered to cells by one or more vectors (e.g., AAV vector).

In some embodiments, one or more vectors (e.g., AAV vector) comprise at least one nucleic acid molecule encoding one or more guide RNAs and a nucleobase editor polypeptide, which comprises a nucleic acid programmable DNA binding protein (napDNAbp) (e.g., Cas9) domain and a deaminase (e.g., cytidine deaminase and/or adenosine deaminase) domain. In some embodiments, one or more vectors (e.g., AAV vector) comprise at least one nucleic acid molecule encoding one or more guide RNAs, which direct a nucleobase editor polypeptide to edit a site in the genome of a cell (e.g., immune cell (e.g., T- or NK-cell)).

The present disclosure provides one or more guide RNAs that direct a nucleobase editor polypeptide to edit a site in the genome of the cell (e.g., immune cell (e.g., T- or NK-cell)). In some embodiments, the present disclosure provides guide RNAs that target components of the peptide loading complex (PLC) (e.g., β2M, TAP1, TAP2, Tapasin) and/or CD58 in an immune cell (e.g., T- or NK-cell). In some embodiments, the present disclosure provides guide RNAs that target β2M, TAP1, TAP2, Tapasin, and/or CD58. In some embodiments, the gRNA comprises nucleotide analogs. These nucleotide analogs can inhibit degradation of the gRNA from cellular processes. Exemplary guide spacer sequences, gRNA sequences, and target sequences are provided in the following Tables 1A-1E and as SEQ ID NOs: 1214-2908, 403-412, and 435-446.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter, but is optionally not added to the guide polynucleotide if or when the guide polynucleotide is not expressed under the control of a promoter.

TABLE 1A
Guide RNAs

| | | | | | | Guide |
|---|---|---|---|---|---|---|
| | | | | | | with |
| | | | | | | Guide |
| | | | | | | Scaf- |
| | | | | Guide | | fold |
| | | | | SEQ | | SEQ |
| Guide | | Base | Spacer | ID | | ID |
| No. | Gene | Editor | Sequence | NO: | gRNA sequence | NO: |
| Guide 443 | TAP1 | ABE/CB | AGACCUGGCUAUGG | 480 | AGACCUGGCUAUGGUGAGAAGUUUUAGAGCUAGAAAU | 575 |
| | | E/CABE | UGAGAA | | AGCAAGUUAAAAUAAGGCUAGUCCGUUAUCAACUUGA | |
| | | | | | AAAAGUGGCACCGAGUCGGUGCUUUU | |
| Guide 444 | TAP1 | CBE | GCACUGCACUAUAA | 481 | GCACUGCACUAUAAAGAACCGUUUUAGAGCUAGAAAU | 576 |
| | | | AGAACC | | AGCAAGUUAAAAUAAGGCUAGUCCGUUAUCAACUUGA | |
| | | | | | AAAAGUGGCACCGAGUCGGUGCUUUU | |
| Guide 445 | TAP1 | ABE | UAGUGCAGUGCUGG | 482 | UAGUGCAGUGCUGGAGUUCGGUUUUAGAGCUAGAAAU | 577 |
| | | | AGUUCG | | AGCAAGUUAAAAUAAGGCUAGUCCGUUAUCAACUUGA | |
| | | | | | AAAAGUGGCACCGAGUCGGUGCUUUU | |
| Guide 446 | TAP1 | ABE/CB | AGAAACCUGUCUGG | 483 | AGAAACCUGUCUGGUUCUGUGUUUUAGAGCUAGAAAU | 578 |
| | | E/CABE | UUCUGU | | AGCAAGUUAAAAUAAGGCUAGUCCGUUAUCAACUUGA | |
| | | | | | AAAAGUGGCACCGAGUCGGUGCUUUU | |
| Guide 447 | TAP1 | CBE | UGAUGUUACCUGCA | 484 | UGAUGUUACCUGCAGGGUUGGUUUUAGAGCUAGAAAU | 579 |
| | | | GGGUUG | | AGCAAGUUAAAAUAAGGCUAGUCCGUUAUCAACUUGA | |
| | | | | | AAAAGUGGCACCGAGUCGGUGCUUUU | |
| Guide 448 | TAP1 | ABE | CUGCAGGUAACAUC | 485 | CUGCAGGUAACAUCAUGUCUGUUUUAGAGCUAGAAAU | 580 |
| | | | AUGUCU | | AGCAAGUUAAAAUAAGGCUAGUCCGUUAUCAACUUGA | |
| | | | | | AAAAGUGGCACCGAGUCGGUGCUUUU | |
| Guide 449 | TAP1 | CBE | ACCAGGUAUGUUCA | 486 | ACCAGGUAUGUUCAUGGAGUGUUUUAGAGCUAGAAAU | 581 |
| | | | UGGAGU | | AGCAAGUUAAAAUAAGGCUAGUCCGUUAUCAACUUGA | |
| | | | | | AAAAGUGGCACCGAGUCGGUGCUUUU | |
| Guide 450 | TAP1 | CBE | UUCCAGCAACUGUG | 487 | UUCCAGCAACUGUGGAUACAGUUUUAGAGCUAGAAAU | 582 |
| | | | GAUACA | | AGCAAGUUAAAAUAAGGCUAGUCCGUUAUCAACUUGA | |
| | | | | | AAAAGUGGCACCGAGUCGGUGCUUUU | |
| Guide 451 | TAP1 | ABE | UCCACAGUUGCUGG | 488 | UCCACAGUUGCUGGAAGUGCGUUUUAGAGCUAGAAAU | 583 |
| | | | AAGUGC | | AGCAAGUUAAAAUAAGGCUAGUCCGUUAUCAACUUGA | |
| | | | | | AAAAGUGGCACCGAGUCGGUGCUUUU | |
| Guide 452 | TAP1 | ABE/CB | GGUGCUCACACUAG | 489 | GGUGCUCACACUAGUGGUCCGUUUUAGAGCUAGAAAU | 584 |
| | | E/CABE | UGGUCC | | AGCAAGUUAAAAUAAGGCUAGUCCGUUAUCAACUUGA | |
| | | | | | AAAAGUGGCACCGAGUCGGUGCUUUU | |
| Guide 453 | TAP1 | ABE/CB | GGGACCUCACCUCC | 490 | GGGACCUCACCUCCACAGCCGUUUUAGAGCUAGAAAU | 585 |
| | | E/CABE | ACAGCC | | AGCAAGUUAAAAUAAGGCUAGUCCGUUAUCAACUUGA | |
| | | | | | AAAAGUGGCACCGAGUCGGUGCUUUU | |
| Guide 454 | TAP1 | ABE/CB | GGACCUCACCUCCA | 491 | GGACCUCACCUCCACAGCCUGUUUUAGAGCUAGAAAU | 586 |
| | | E/CABE | CAGCCU | | AGCAAGUUAAAAUAAGGCUAGUCCGUUAUCAACUUGA | |
| | | | | | AAAAGUGGCACCGAGUCGGUGCUUUU | |
| Guide 455 | TAP1 | ABE/CB | AUACCUGCCUGUGC | 492 | AUACCUGCCUGUGCAGGUAGGUUUUAGAGCUAGAAAU | 587 |
| | | E | AGGUAG | | AGCAAGUUAAAAUAAGGCUAGUCCGUUAUCAACUUGA | |
| | | | | | AAAAGUGGCACCGAGUCGGUGCUUUU | |
| Guide 456 | TAP1 | ABE/CB | GCUUCCAUACCUGC | 493 | GCUUCCAUACCUGCCUGUGCGUUUUAGAGCUAGAAAU | 588 |
| | | E/CABE | CUGUGC | | AGCAAGUUAAAAUAAGGCUAGUCCGUUAUCAACUUGA | |
| | | | | | AAAAGUGGCACCGAGUCGGUGCUUUU | |
| Guide 457 | TAP1 | ABE | UCAUCUCAGGUGGC | 494 | UCAUCUCAGGUGGCUGCAGUGUUUUAGAGCUAGAAAU | 589 |
| | | | UGCAGU | | AGCAAGUUAAAAUAAGGCUAGUCCGUUAUCAACUUGA | |
| | | | | | AAAAGUGGCACCGAGUCGGUGCUUUU | |
| Guide 458 | TAP1 | ABE | UUCAUCUCAGGUGG | 495 | UUCAUCUCAGGUGGCUGCAGGUUUUAGAGCUAGAAAU | 590 |
| | | | CUGCAG | | AGCAAGUUAAAAUAAGGCUAGUCCGUUAUCAACUUGA | |
| | | | | | AAAAGUGGCACCGAGUCGGUGCUUUU | |
| Guide 459 | TAP1 | ABE/CB | GUACCUGUGUCAUA | 496 | GUACCUGUGUCAUAGCCCUGGUUUUAGAGCUAGAAAU | 591 |
| | | E/CABE | GCCCUG | | AGCAAGUUAAAAUAAGGCUAGUCCGUUAUCAACUUGA | |
| | | | | | AAAAGUGGCACCGAGUCGGUGCUUUU | |
| Guide 460 | TAP1 | CBE | GUCUACCUCUGCAG | 497 | GUCUACCUCUGCAGAGCAAAGUUUUAGAGCUAGAAAU | 592 |
| | | | AGCAAA | | AGCAAGUUAAAAUAAGGCUAGUCCGUUAUCAACUUGA | |
| | | | | | AAAAGUGGCACCGAGUCGGUGCUUUU | |
| Guide 461 | TAP1 | CBE | CGUCUACCUCUGCA | 498 | CGUCUACCUCUGCAGAGCAAGUUUUAGAGCUAGAAAU | 593 |
| | | | GAGCAA | | AGCAAGUUAAAAUAAGGCUAGUCCGUUAUCAACUUGA | |
| | | | | | AAAAGUGGCACCGAGUCGGUGCUUUU | |
| Guide 462 | TAP1 | ABE | CUGCAGAGGUAGAC | 499 | CUGCAGAGGUAGACGAGGCUGUUUUAGAGCUAGAAAU | 594 |
| | | | GAGGCU | | AGCAAGUUAAAAUAAGGCUAGUCCGUUAUCAACUUGA | |
| | | | | | AAAAGUGGCACCGAGUCGGUGCUUUU | |
| Guide 463 | TAP1 | ABE | UCUGCAGAGGUAGA | 500 | UCUGCAGAGGUAGACGAGGCGUUUUAGAGCUAGAAAU | 595 |
| | | | CGAGGC | | AGCAAGUUAAAAUAAGGCUAGUCCGUUAUCAACUUGA | |
| | | | | | AAAAGUGGCACCGAGUCGGUGCUUUU | |
| Guide 464 | TAP1 | ABE | UUGCUCUGCAGAGG | 501 | UUGCUCUGCAGAGGUAGACGGUUUUAGAGCUAGAAAU | 596 |
| | | | UAGACG | | AGCAAGUUAAAAUAAGGCUAGUCCGUUAUCAACUUGA | |
| | | | | | AAAAGUGGCACCGAGUCGGUGCUUUU | |
| Guide 465 | TAP1 | ABE | CUCCACCUGAGGAA | 502 | CUCCACCUGAGGAAAGACAUGUUUUAGAGCUAGAAAU | 597 |
| | | | AGACAU | | AGCAAGUUAAAAUAAGGCUAGUCCGUUAUCAACUUGA | |
| | | | | | AAAAGUGGCACCGAGUCGGUGCUUUU | |
| Guide 466 | TAP2 | ABE/CB | CUGACUCACCCAAA | 503 | CUGACUCACCCAAAACAGCAGUUUUAGAGCUAGAAAU | 598 |
| | | E/CABE | ACAGCA | | AGCAAGUUAAAAUAAGGCUAGUCCGUUAUCAACUUGA | |
| | | | | | AAAAGUGGCACCGAGUCGGUGCUUUU | |
| Guide 467 | TAP2 | ABE/CB | CACCCAAAACAGCA | 504 | CACCCAAAACAGCAAGGACAGUUUUAGAGCUAGAAAU | 599 |
| | | E/CABE | AGGACA | | AGCAAGUUAAAAUAAGGCUAGUCCGUUAUCAACUUGA | |
| | | | | | AAAAGUGGCACCGAGUCGGUGCUUUU | |
| Guide 468 | TAP2 | ABE/CB | CCUACCUGCCAAAG | 505 | CCUACCUGCCAAAGGAGAAGGUUUUAGAGCUAGAAAU | 600 |
| | | E/CABE | GAGAAG | | AGCAAGUUAAAAUAAGGCUAGUCCGUUAUCAACUUGA | |
| | | | | | AAAAGUGGCACCGAGUCGGUGCUUUU | |
| Guide 469 | TAP2 | ABE | CCCCACAGCUCACU | 506 | CCCCACAGCUCACUGUCUGCGUUUUAGAGCUAGAAAU | 601 |
| | | | GUCUGC | | AGCAAGUUAAAAUAAGGCUAGUCCGUUAUCAACUUGA | |
| | | | | | AAAAGUGGCACCGAGUCGGUGCUUUU | |
| Guide 470 | TAP2 | CBE | CAGACAGUGAGCUG | 507 | CAGACAGUGAGCUGUGGGGUGUUUUAGAGCUAGAAAU | 602 |
| | | | UGGGGU | | AGCAAGUUAAAAUAAGGCUAGUCCGUUAUCAACUUGA | |
| | | | | | AAAAGUGGCACCGAGUCGGUGCUUUU | |
| Guide 471 | TAP2 | ABE/ | CCCCACCUGUCUUA | 508 | CCCCACCUGUCUUAGUCUCCGUUUUAGAGCUAGAAAU | 603 |
| | | CBE/ | GUCUCC | | AGCAAGUUAAAAUAAGGCUAGUCCGUUAUCAACUUGA | |
| | | CABE | | | AAAAGUGGCACCGAGUCGGUGCUUUU | |
| Guide 472 | TAP2 | ABE | UCUUAGGGGAGCUG | 509 | UCUUAGGGGAGCUGAACUCAGUUUUAGAGCUAGAAAU | 604 |
| | | | AACUCA | | AGCAAGUUAAAAUAAGGCUAGUCCGUUAUCAACUUGA | |
| | | | | | AAAAGUGGCACCGAGUCGGUGCUUUU | |
| Guide 473 | TAP2 | CBE | UCCCCUAAGAAGGA | 510 | UCCCCUAAGAAGGACAGAGCGUUUUAGAGCUAGAAAU | 605 |
| | | | CAGAGC | | AGCAAGUUAAAAUAAGGCUAGUCCGUUAUCAACUUGA | |
| | | | | | AAAAGUGGCACCGAGUCGGUGCUUUU | |
| Guide 474 | TAP2 | CBE | AUCAGGUGAGCGUG | 511 | AUCAGGUGAGCGUGCAUGUAGUUUUAGAGCUAGAAAU | 606 |
| | | | CAUGUA | | AGCAAGUUAAAAUAAGGCUAGUCCGUUAUCAACUUGA | |
| | | | | | AAAAGUGGCACCGAGUCGGUGCUUUU | |
| Guide 475 | TAP2 | ABE | UCUUCCAGGUGCUG | 512 | UCUUCCAGGUGCUGCACUUGGUUUUAGAGCUAGAAAU | 607 |
| | | | CACUUG | | AGCAAGUUAAAAUAAGGCUAGUCCGUUAUCAACUUGA | |
| | | | | | AAAAGUGGCACCGAGUCGGUGCUUUU | |
| Guide 476 | TAP2 | ABE | CUCUUCCAGGUGCU | 513 | CUCUUCCAGGUGCUGCACUUGUUUUAGAGCUAGAAAU | 608 |
| | | | GCACUU | | AGCAAGUUAAAAUAAGGCUAGUCCGUUAUCAACUUGA | |
| | | | | | AAAAGUGGCACCGAGUCGGUGCUUUU | |
| Guide 477 | TAP2 | ABE | CCUCUUCCAGGUGC | 514 | CCUCUUCCAGGUGCUGCACUGUUUUAGAGCUAGAAAU | 609 |
| | | | UGCACU | | AGCAAGUUAAAAUAAGGCUAGUCCGUUAUCAACUUGA | |
| | | | | | AAAAGUGGCACCGAGUCGGUGCUUUU | |
| Guide 478 | TAP2 | ABE | CUUCCAGGUGCUGC | 515 | CUUCCAGGUGCUGCACUUGGGUUUUAGAGCUAGAAAU | 610 |
| | | | ACUUGG | | AGCAAGUUAAAAUAAGGCUAGUCCGUUAUCAACUUGA | |
| | | | | | AAAAGUGGCACCGAGUCGGUGCUUUU | |
| Guide 479 | TAP2 | CBE | CAGGGUCUGGAAAA | 516 | CAGGGUCUGGAAAACAGGAAGUUUUAGAGCUAGAAAU | 611 |
| | | | CAGGAA | | AGCAAGUUAAAAUAAGGCUAGUCCGUUAUCAACUUGA | |
| | | | | | AAAAGUGGCACCGAGUCGGUGCUUUU | |
| Guide 480 | TAP2 | CBE | AGGGUCUGGAAAAC | 517 | AGGGUCUGGAAAACAGGAAUGUUUUAGAGCUAGAAAU | 612 |
| | | | AGGAAU | | AGCAAGUUAAAAUAAGGCUAGUCCGUUAUCAACUUGA | |
| | | | | | AAAAGUGGCACCGAGUCGGUGCUUUU | |
| Guide 481 | TAP2 | ABE/CB | CUUUCAGGCACCUU | 518 | CUUUCAGGCACCUUGAGCACGUUUUAGAGCUAGAAAU | 613 |
| | | E/CABE | GAGCAC | | AGCAAGUUAAAAUAAGGCUAGUCCGUUAUCAACUUGA | |
| | | | | | AAAAGUGGCACCGAGUCGGUGCUUUU | |
| Guide 482 | TAP2 | ABE/CB | CCUCACCCACCUGG | 519 | CCUCACCCACCUGGCUGUGCGUUUUAGAGCUAGAAAU | 614 |
| | | E/CABE | CUGUGC | | AGCAAGUUAAAAUAAGGCUAGUCCGUUAUCAACUUGA | |
| | | | | | AAAAGUGGCACCGAGUCGGUGCUUUU | |
| Guide 483 | TAP2 | ABE | CUGCACAGGUGGUU | 520 | CUGCACAGGUGGUUUCAGUUGUUUUAGAGCUAGAAAU | 615 |
| | | | UCAGUU | | AGCAAGUUAAAAUAAGGCUAGUCCGUUAUCAACUUGA | |
| | | | | | AAAAGUGGCACCGAGUCGGUGCUUUU | |
| Guide 484 | TAP2 | ABE | GCUGCACAGGUGGU | 521 | GCUGCACAGGUGGUUUCAGUGUUUUAGAGCUAGAAAU | 616 |
| | | | UUCAGU | | AGCAAGUUAAAAUAAGGCUAGUCCGUUAUCAACUUGA | |
| | | | | | AAAAGUGGCACCGAGUCGGUGCUUUU | |
| Guide 485 | TAP2 | CBE | CCACCUGUGCAGCA | 522 | CCACCUGUGCAGCAGGGACAGUUUUAGAGCUAGAAAU | 617 |
| | | | GGGACA | | AGCAAGUUAAAAUAAGGCUAGUCCGUUAUCAACUUGA | |
| | | | | | AAAAGUGGCACCGAGUCGGUGCUUUU | |
| Guide 486 | TAP2 | CBE | CACCUGUGCAGCAG | 523 | CACCUGUGCAGCAGGGACAGGUUUUAGAGCUAGAAAU | 618 |
| | | | GGACAG | | AGCAAGUUAAAAUAAGGCUAGUCCGUUAUCAACUUGA | |
| | | | | | AAAAGUGGCACCGAGUCGGUGCUUUU | |
| Guide 487 | TAP2 | CBE | ACCACCUGUGCAGC | 524 | ACCACCUGUGCAGCAGGGACGUUUUAGAGCUAGAAAU | 619 |
| | | | AGGGAC | | AGCAAGUUAAAAUAAGGCUAGUCCGUUAUCAACUUGA | |
| | | | | | AAAAGUGGCACCGAGUCGGUGCUUUU | |
| Guide 488 | TAP2 | ABE | UCCUCAGAUGUAGG | 525 | UCCUCAGAUGUAGGGGAGAAGUUUUAGAGCUAGAAAU | 620 |
| | | | GGAGAA | | AGCAAGUUAAAAUAAGGCUAGUCCGUUAUCAACUUGA | |
| | | | | | AAAAGUGGCACCGAGUCGGUGCUUUU | |
| Guide 489 | TAP2 | ABE | UUCCUCAGAUGUAG | 526 | UUCCUCAGAUGUAGGGGAGAGUUUUAGAGCUAGAAAU | 621 |
| | | | GGGAGA | | AGCAAGUUAAAAUAAGGCUAGUCCGUUAUCAACUUGA | |
| | | | | | AAAAGUGGCACCGAGUCGGUGCUUUU | |
| Guide 518 | TAP1.1 | ABE | UCGCCGACUGGGUG | 527 | UCGCCGACUGGGUGCUGCUCGUUUUAGAGCUAGAAAU | 622 |
| | | | CUGCUC | | AGCAAGUUAAAAUAAGGCUAGUCCGUUAUCAACUUGA | |
| | | | | | AAAAGUGGCACCGAGUCGGUGCUUUU | |
| Guide 519 | TAP1.2 | CBE | GUGCUCUUCCCAGA | 528 | GUGCUCUUCCCAGACCCAUUGUUUUAGAGCUAGAAAU | 623 |
| | AND | | CCCAUU | | AGCAAGUUAAAAUAAGGCUAGUCCGUUAUCAACUUGA | |
| | TAP2 | | | | AAAAGUGGCACCGAGUCGGUGCUUUU | |
| Guide 520 | TAP1.3 | CBE | UGUGCUCUUCCCAG | 529 | UGUGCUCUUCCCAGACCCAUGUUUUAGAGCUAGAAAU | 624 |
| | AND TAP | | ACCCAU | | AGCAAGUUAAAAUAAGGCUAGUCCGUUAUCAACUUGA | |
| | 2 | | | | AAAAGUGGCACCGAGUCGGUGCUUUU | |
| Guide 521 | TAP1.4 | CBE | UGGGUCUGGGAAGA | 530 | UGGGUCUGGGAAGAGCACAGGUUUUAGAGCUAGAAAU | 625 |
| | | | GCACAG | | AGCAAGUUAAAAUAAGGCUAGUCCGUUAUCAACUUGA | |
| | | | | | AAAAGUGGCACCGAGUCGGUGCUUUU | |
| Guide 522 | TAP1.5 | CBE | GUCGCUGACCCCCU | 531 | GUCGCUGACCCCCUGACAGCGUUUUAGAGCUAGAAAU | 626 |
| | | | GACAGC | | AGCAAGUUAAAAUAAGGCUAGUCCGUUAUCAACUUGA | |
| | | | | | AAAAGUGGCACCGAGUCGGUGCUUUU | |
| Guide 523 | TAP1.6 | CBE, | UCAGCGACAGGCAG | 532 | UCAGCGACAGGCAGUGGCGUGUUUUAGAGCUAGAAAU | 627 |
| | | ABE | UGGCGU | | AGCAAGUUAAAAUAAGGCUAGUCCGUUAUCAACUUGA | |
| | | | | | AAAAGUGGCACCGAGUCGGUGCUUUU | |
| Guide 524 | TAP1.7 | ABE | GGAUAAGUACACAC | 533 | GGAUAAGUACACACGGUUUCGUUUUAGAGCUAGAAAU | 628 |
| | | | GGUUUC | | AGCAAGUUAAAAUAAGGCUAGUCCGUUAUCAACUUGA | |
| | | | | | AAAAGUGGCACCGAGUCGGUGCUUUU | |
| Guide 525 | TAP1.8 | CBE, | UCAUCCAGGAUAAG | 534 | UCAUCCAGGAUAAGUACACAGUUUUAGAGCUAGAAAU | 629 |
| | | ABE | UACACA | | AGCAAGUUAAAAUAAGGCUAGUCCGUUAUCAACUUGA | |
| | | | | | AAAAGUGGCACCGAGUCGGUGCUUUU | |
| Guide 526 | TAP1.9 | CBE, | GGGCACUGGUGGCA | 535 | GGGCACUGGUGGCAUCAUCCGUUUUAGAGCUAGAAAU | 630 |
| | | ABE | UCAUCC | | AGCAAGUUAAAAUAAGGCUAGUCCGUUAUCAACUUGA | |
| | | | | | AAAAGUGGCACCGAGUCGGUGCUUUU | |
| Guide 527 | TAP1.10 | CBE, | GUUUGCAUCCAGGG | 536 | GUUUGCAUCCAGGGCACUGGGUUUUAGAGCUAGAAAU | 631 |
| | | ABE | CACUGG | | AGCAAGUUAAAAUAAGGCUAGUCCGUUAUCAACUUGA | |
| | | | | | AAAAGUGGCACCGAGUCGGUGCUUUU | |
| Guide 528 | TAP1.11 | ABE | CAUCACCCAGCACC | 537 | CAUCACCCAGCACCUCAGCCGUUUUAGAGCUAGAAAU | 632 |
| | | | UCAGCC | | AGCAAGUUAAAAUAAGGCUAGUCCGUUAUCAACUUGA | |
| | | | | | AAAAGUGGCACCGAGUCGGUGCUUUU | |
| Guide 529 | TAP1.12 | ABE | CACCCAGCACCUCA | 538 | CACCCAGCACCUCAGCCUGGGUUUUAGAGCUAGAAAU | 633 |
| | | | GCCUGG | | AGCAAGUUAAAAUAAGGCUAGUCCGUUAUCAACUUGA | |
| | | | | | AAAAGUGGCACCGAGUCGGUGCUUUU | |
| Guide 530 | TAP2.1 | ABE | GGUGGACGCGGCUU | 539 | GGUGGACGCGGCUUUACUGGUUUUAGAGCUAGAAAUA | 634 |
| | | | UACUG | | GCAAGUUAAAAUAAGGCUAGUCCGUUAUCAACUUGAA | |
| | | | | | AAAGUGGCACCGAGUCGGUGCUUUU | |
| Guide 531 | TAP2.4 | ABE | UGCGGGACAGAAAC | 540 | UGCGGGACAGAAACAACGUCGUUUUAGAGCUAGAAAU | 635 |
| | | | AACGUC | | AGCAAGUUAAAAUAAGGCUAGUCCGUUAUCAACUUGA | |
| | | | | | AAAAGUGGCACCGAGUCGGUGCUUUU | |
| Guide 532 | TAP2.5 | CBE, | GGACCCGCGGGUCU | 541 | GGACCCGCGGGUCUCGUACAGUUUUAGAGCUAGAAAU | 636 |
| | | ABE | CGUACA | | AGCAAGUUAAAAUAAGGCUAGUCCGUUAUCAACUUGA | |
| | | | | | AAAAGUGGCACCGAGUCGGUGCUUUU | |
| Guide 533 | TAP2.6 | CBE, | CAUCCAGGAUGAGG | 542 | CAUCCAGGAUGAGGACCCGCGUUUUAGAGCUAGAAAU | 637 |
| | | ABE | ACCCGC | | AGCAAGUUAAAAUAAGGCUAGUCCGUUAUCAACUUGA | |
| | | | | | AAAAGUGGCACCGAGUCGGUGCUUUU | |
| Guide 534 | TAP2.7 | CBE, | UCAUCCAGGAUGAG | 543 | UCAUCCAGGAUGAGGACCCGGUUUUAGAGCUAGAAAU | 638 |
| | | ABE | GACCCG | | AGCAAGUUAAAAUAAGGCUAGUCCGUUAUCAACUUGA | |
| | | | | | AAAAGUGGCACCGAGUCGGUGCUUUU | |
| Guide 535 | TAP2.8 | CBE | GCGGGUCCUCAUCC | 544 | GCGGGUCCUCAUCCUGGAUGGUUUUAGAGCUAGAAAU | 639 |
| | | | UGGAUG | | AGCAAGUUAAAAUAAGGCUAGUCCGUUAUCAACUUGA | |
| | | | | | AAAAGUGGCACCGAGUCGGUGCUUUU | |
| Guide 536 | TAP2.9 | CBE, | GGGCACUAGUAGCC | 545 | GGGCACUAGUAGCCUCAUCCGUUUUAGAGCUAGAAAU | 640 |
| | | ABE | UCAUCC | | AGCAAGUUAAAAUAAGGCUAGUCCGUUAUCAACUUGA | |
| | | | | | AAAAGUGGCACCGAGUCGGUGCUUUU | |
| Guide 537 | TAP2.10 | ABE | CCUAGAUGUGCAGU | 546 | CCUAGAUGUGCAGUGCGAGCGUUUUAGAGCUAGAAAU | 641 |
| | | | GCGAGC | | AGCAAGUUAAAAUAAGGCUAGUCCGUUAUCAACUUGA | |
| | | | | | AAAAGUGGCACCGAGUCGGUGCUUUU | |
| Guide 415 | ß2M- | CBE, | ACUCACGCUGGAUA | 547 | ACUCACGCUGGAUAGCCUCCGUUUUAGAGCUAGAAAU | 642 |
| | 20mer | ABE | GCCUCC | | AGCAAGUUAAAAUAAGGCUAGUCCGUUAUCAACUUGA | |
| | | | | | AAAAGUGGCACCGAGUCGGUGCUUUU | |
| Guide 416 | ß2M | CBE | CACAGCCCAAGAUA | 548 | CACAGCCCAAGAUAGUUAAGGUUUUAGAGCUAGAAAU | 643 |
| | | | GUUAAG | | AGCAAGUUAAAAUAAGGCUAGUCCGUUAUCAACUUGA | |
| | | | | | AAAAGUGGCACCGAGUCGGUGCUUUU | |
| Guide 417 | ß2M | CBE | ACAGCCCAAGAUAG | 549 | ACAGCCCAAGAUAGUUAAGUGUUUUAGAGCUAGAAAU | 644 |
| (b2m 315 | | | UUAAGU | | AGCAAGUUAAAAUAAGGCUAGUCCGUUAUCAACUUGA | |
| ) | | | | | AAAAGUGGCACCGAGUCGGUGCUUUU | |
| Guide 418 | ß2M | CBE | CAGCCCAAGAUAGU | 550 | CAGCCCAAGAUAGUUAAGUGGUUUUAGAGCUAGAAAU | 645 |
| (b2m_316 | | | UAAGUG | | AGCAAGUUAAAAUAAGGCUAGUCCGUUAUCAACUUGA | |
| ) | | | | | AAAAGUGGCACCGAGUCGGUGCUUUU | |
| Guide 419 | B2M | CBE, | UUACCCCACUUAAC | 551 | UUACCCCACUUAACUAUCUUGUUUUAGAGCUAGAAAU | 646 |
| | | ABE | UAUCUU | | AGCAAGUUAAAAUAAGGCUAGUCCGUUAUCAACUUGA | |
| | | | | | AAAAGUGGCACCGAGUCGGUGCUUUU | |
| Guide 420 | ß2M | CBE. | CUUACCCCACUUAA | 552 | CUUACCCCACUUAACUAUCUGUUUUAGAGCUAGAAAU | 647 |
| (TSBTx76 | | ABE | CUAUCU | | AGCAAGUUAAAAUAAGGCUAGUCCGUUAUCAACUUGA | |
| 0) | | | | | AAAAGUGGCACCGAGUCGGUGCUUUU | |
| Guide 421 | B2M | ABE | UCGAUCUAUGAAAA | 553 | UCGAUCUAUGAAAAAGACAGGUUUUAGAGCUAGAAAU | 648 |
| | | | AGACAG | | AGCAAGUUAAAAUAAGGCUAGUCCGUUAUCAACUUGA | |
| | | | | | AAAAGUGGCACCGAGUCGGUGCUUUU | |
| Guide 422 | B2M- | CBE, | CUCACGCUGGAUAG | 554 | CUCACGCUGGAUAGCCUCCGUUUUAGAGCUAGAAAUA | 649 |
| | 19mer | ABE | CCUCC | | GCAAGUUAAAAUAAGGCUAGUCCGUUAUCAACUUGAA | |
| | | | | | AAAGUGGCACCGAGUCGGUGCUUUU | |
| Guide 423 | ß2M- | CBE, | UCACGCUGGAUAGC | 555 | UCACGCUGGAUAGCCUCCGUUUUAGAGCUAGAAAUAG | 650 |
| | 18mer | ABE | CUCC | | CAAGUUAAAAUAAGGCUAGUCCGUUAUCAACUUGAAA | |
| | | | | | AAGUGGCACCGAGUCGGUGCUUUU | |
| Guide 424 | ?2M | CBE, | CGCGAGCACAGCUA | 556 | CGCGAGCACAGCUAAGGCCAGUUUUAGAGCUAGAAAU | 651 |
| | | ABE | AGGCCA | | AGCAAGUUAAAAUAAGGCUAGUCCGUUAUCAACUUGA | |
| | | | | | AAAAGUGGCACCGAGUCGGUGCUUUU | |
| Guide 425 | ß2M | CBE, | GAGUAGCGCGAGCA | 557 | GAGUAGCGCGAGCACAGCUAGUUUUAGAGCUAGAAAU | 652 |
| | | ABE | CAGCUA | | AGCAAGUUAAAAUAAGGCUAGUCCGUUAUCAACUUGA | |
| | | | | | AAAAGUGGCACCGAGUCGGUGCUUUU | |
| Guide 426 | ?2M | CBE, | GCUACUCUCUCUUU | 558 | GCUACUCUCUCUUUCUGGCCGUUUUAGAGCUAGAAAU | 653 |
| | | ABE | CUGGCC | | AGCAAGUUAAAAUAAGGCUAGUCCGUUAUCAACUUGA | |
| | | | | | AAAAGUGGCACCGAGUCGGUGCUUUU | |
| Guide 427 | ß2M | CBE | CGUGAGUAAACCUG | 559 | CGUGAGUAAACCUGAAUCUUGUUUUAGAGCUAGAAAU | 654 |
| | | | AAUCUU | | AGCAAGUUAAAAUAAGGCUAGUCCGUUAUCAACUUGA | |
| | | | | | AAAAGUGGCACCGAGUCGGUGCUUUU | |
| Guide 428 | ß2M | CBE, | UUUGACUUUCCAUU | 560 | UUUGACUUUCCAUUCUCUGCGUUUUAGAGCUAGAAAU | 655 |
| | | ABE | CUCUGC | | AGCAAGUUAAAAUAAGGCUAGUCCGUUAUCAACUUGA | |
| | | | | | AAAAGUGGCACCGAGUCGGUGCUUUU | |
| Guide 429 | ?2M | CBE, | ACCCAGACACAUAG | 561 | ACCCAGACACAUAGCAAUUCGUUUUAGAGCUAGAAAU | 656 |
| | | ABE | CAAUUC | | AGCAAGUUAAAAUAAGGCUAGUCCGUUAUCAACUUGA | |
| | | | | | AAAAGUGGCACCGAGUCGGUGCUUUU | |
| Guide 430 | ß2M | CBE, | AAGUCAACUUCAAU | 562 | AAGUCAACUUCAAUGUCGGAGUUUUAGAGCUAGAAAU | 657 |
| | | ABE | GUCGGA | | AGCAAGUUAAAAUAAGGCUAGUCCGUUAUCAACUUGA | |
| | | | | | AAAAGUGGCACCGAGUCGGUGCUUUU | |
| Guide 431 | ?2M | CBE, | CAGUAAGUCAACUU | 563 | CAGUAAGUCAACUUCAAUGUGUUUUAGAGCUAGAAAU | 658 |
| (b2m_629 | | ABE | CAAUGU | | AGCAAGUUAAAAUAAGGCUAGUCCGUUAUCAACUUGA | |
| - | | | | | AAAAGUGGCACCGAGUCGGUGCUUUU | |
| Guide 432 | ß2M | CBE, | GAAGUUGACUUACU | 564 | GAAGUUGACUUACUGAAGAAGUUUUAGAGCUAGAAAU | 659 |
| | | ABE | GAAGAA | | AGCAAGUUAAAAUAAGGCUAGUCCGUUAUCAACUUGA | |
| | | | | | AAAAGUGGCACCGAGUCGGUGCUUUU | |
| Guide 433 | ß2M | CBE, | UGGAGAGAGAAUUG | 565 | UGGAGAGAGAAUUGAAAAAGGUUUUAGAGCUAGAAAU | 660 |
| | | ABE | AAAAAG | | AGCAAGUUAAAAUAAGGCUAGUCCGUUAUCAACUUGA | |
| | | | | | AAAAGUGGCACCGAGUCGGUGCUUUU | |
| Guide 434 | ß2M | CBE, | UUCAGACUUGUCUU | 566 | UUCAGACUUGUCUUUCAGCAGUUUUAGAGCUAGAAAU | 661 |
| | | ABE | UCAGCA | | AGCAAGUUAAAAUAAGGCUAGUCCGUUAUCAACUUGA | |
| | | | | | AAAAGUGGCACCGAGUCGGUGCUUUU | |
| Guide 435 | ?2M | CBE, | ACUUGUCUUUCAGC | 567 | ACUUGUCUUUCAGCAAGGACGUUUUAGAGCUAGAAAU | 662 |
| | | ABE | AAGGAC | | AGCAAGUUAAAAUAAGGCUAGUCCGUUAUCAACUUGA | |
| | | | | | AAAAGUGGCACCGAGUCGGUGCUUUU | |
| Guide 436 | ?2M | CBE, | AUACUCAUCUUUUU | 568 | AUACUCAUCUUUUUCAGUGGGUUUUAGAGCUAGAAAU | 663 |
| | | ABE | CAGUGG | | AGCAAGUUAAAAUAAGGCUAGUCCGUUAUCAACUUGA | |
| | | | | | AAAAGUGGCACCGAGUCGGUGCUUUU | |
| Guide 437 | ß2M | CBE, | CAUACUCAUCUUUU | 569 | CAUACUCAUCUUUUUCAGUGGUUUUAGAGCUAGAAAU | 664 |
| | | ABE | UCAGUG | | AGCAAGUUAAAAUAAGGCUAGUCCGUUAUCAACUUGA | |
| | | | | | AAAAGUGGCACCGAGUCGGUGCUUUU | |
| Guide 438 | ß2M | CBE, | GCAUACUCAUCUUU | 570 | GCAUACUCAUCUUUUUCAGUGUUUUAGAGCUAGAAAU | 665 |
| | | ABE | UUCAGU | | AGCAAGUUAAAAUAAGGCUAGUCCGUUAUCAACUUGA | |
| | | | | | AAAAGUGGCACCGAGUCGGUGCUUUU | |
| Guide 439 | ?2M | CBE, | GGCAUACUCAUCUU | 571 | GGCAUACUCAUCUUUUUCAGGUUUUAGAGCUAGAAAU | 666 |
| | | ABE | UUUCAG | | AGCAAGUUAAAAUAAGGCUAGUCCGUUAUCAACUUGA | |
| | | | | | AAAAGUGGCACCGAGUCGGUGCUUUU | |
| Guide 440 | B2M | CBE, | AGUCACAUGGUUCA | 572 | AGUCACAUGGUUCACACGGCGUUUUAGAGCUAGAAAU | 667 |
| | | ABE | CACGGC | | AGCAAGUUAAAAUAAGGCUAGUCCGUUAUCAACUUGA | |
| | | | | | AAAAGUGGCACCGAGUCGGUGCUUUU | |
| Guide 441 | ß2M | CBE, | ACAAAGUCACAUGG | 573 | ACAAAGUCACAUGGUUCACAGUUUUAGAGCUAGAAAU | 668 |
| | | ABE | UUCACA | | AGCAAGUUAAAAUAAGGCUAGUCCGUUAUCAACUUGA | |
| | | | | | AAAAGUGGCACCGAGUCGGUGCUUUU | |
| Guide 442 | ß2M | CBE, | UGGGCUGUGACAAA | 574 | UGGGCUGUGACAAAGUCACAGUUUUAGAGCUAGAAAU | 669 |
| | | ABE | GUCACA | | AGCAAGUUAAAAUAAGGCUAGUCCGUUAUCAACUUGA | |
| | | | | | AAAAGUGGCACCGAGUCGGUGCUUUU | |
| TSBTx36 | CD48 | ABE | AAGCAUGUGCUCCA | 678 | AAGCAUGUGCUCCAGAGGUUGUUUUAGAGCUAGAAAU | 845 |
| 33 | | | GAGGUU | | AGCAAGUUAAAAUAAGGCUAGUCCGUUAUCAACUUGA | |
| | | | | | AAAAGUGGCACCGAGUCGGUGCUUUU | |
| TSBTx36 | CD48 | ABE | GAAGCAUGUGCUCC | 679 | GAAGCAUGUGCUCCAGAGGUGUUUUAGAGCUAGAAAU | 846 |
| 34 | | | AGAGGU | | AGCAAGUUAAAAUAAGGCUAGUCCGUUAUCAACUUGA | |
| | | | | | AAAAGUGGCACCGAGUCGGUGCUUUU | |
| TSBTx36 | CD48 | CBE | UUCUUACCCAGGGU | 680 | UUCUUACCCAGGGUACAGGGGUUUUAGAGCUAGAAAU | 847 |
| 35 | | | ACAGGG | | AGCAAGUUAAAAUAAGGCUAGUCCGUUAUCAACUUGA | |
| | | | | | AAAAGUGGCACCGAGUCGGUGCUUUU | |
| TSBTx36 | CD48 | ABE | UUCUUACCCAGGGU | 681 | UUCUUACCCAGGGUACAGGGGUUUUAGAGCUAGAAAU | 848 |
| 35 | | | ACAGGG | | AGCAAGUUAAAAUAAGGCUAGUCCGUUAUCAACUUGA | |
| | | | | | AAAAGUGGCACCGAGUCGGUGCUUUU | |
| TSBTx36 | CD48 | CBE | CGACCAGAAGAUUG | 682 | CGACCAGAAGAUUGUAGAAUGUUUUAGAGCUAGAAAU | 849 |
| 36 | | | UAGAAU | | AGCAAGUUAAAAUAAGGCUAGUCCGUUAUCAACUUGA | |
| | | | | | AAAAGUGGCACCGAGUCGGUGCUUUU | |
| TSBTx36 | CD48 | CBE | UCGACCAGAAGAUU | 683 | UCGACCAGAAGAUUGUAGAAGUUUUAGAGCUAGAAAU | 850 |
| 37 | | | GUAGAA | | AGCAAGUUAAAAUAAGGCUAGUCCGUUAUCAACUUGA | |
| | | | | | AAAAGUGGCACCGAGUCGGUGCUUUU | |
| TSBTx36 | CD48 | CBE | AAUCCCAUUCUACA | 684 | AAUCCCAUUCUACAAUCUUCGUUUUAGAGCUAGAAAU | 851 |
| 38 | | | AUCUUC | | AGCAAGUUAAAAUAAGGCUAGUCCGUUAUCAACUUGA | |
| | | | | | AAAAGUGGCACCGAGUCGGUGCUUUU | |
| TSBTx36 | CD48 | CBE | UGCAAUCCAUUCUA | 685 | UGCAAUCCAUUCUACUCCAAGUUUUAGAGCUAGAAAU | 852 |
| 39 | | | CUCCAA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | GGUCCAUGAGGAGC | 686 | GGUCCAUGAGGAGCCAGCCCGUUUUAGAGCUAGAAAU | 853 |
| 40 | (CITA) | | CAGCCC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | UGCAGGGUUCACCA | 687 | UGCAGGGUUCACCAGCCAGCGUUUUAGAGCUAGAAAU | 854 |
| 41 | (CITA) | | GCCAGC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | CGAAAACUGCGAGG | 688 | CGAAAACUGCGAGGAGACCAGUUUUAGAGCUAGAAAU | 855 |
| 42 | (CITA) | | AGACCA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | AGGCUGCAAGAUUG | 689 | AGGCUGCAAGAUUGUGGCAAGUUUUAGAGCUAGAAAU | 856 |
| 43 | (CITA) | | UGGCAA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | UGCCAGAGCUCCAG | 690 | UGCCAGAGCUCCAGACCUGCGUUUUAGAGCUAGAAAU | 857 |
| 44 | (CITA) | | ACCUGC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | AUACUCACUCCACU | 691 | AUACUCACUCCACUUCCUCCGUUUUAGAGCUAGAAAU | 858 |
| 45 | (CITA) | | UCCUCC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | AUACUCACUCCACU | 692 | AUACUCACUCCACUUCCUCCGUUUUAGAGCUAGAAAU | 859 |
| 45 | (CITA) | | UCCUCC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | AGGCUGUGGAGAGC | 693 | AGGCUGUGGAGAGCAGGAGAGUUUUAGAGCUAGAAAU | 860 |
| 46 | (CITA) | | AGGAGA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | UCCACAGCCUCAGU | 694 | UCCACAGCCUCAGUAACAACGUUUUAGAGCUAGAAAU | 861 |
| 47 | (CITA) | | AACAAC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | GCAGGCUGUGAAGA | 695 | GCAGGCUGUGAAGACAAAAGGUUUUAGAGCUAGAAAU | 862 |
| 48 | (CITA) | | CAAAAG | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | CAGGCUGUGAAGAC | 696 | CAGGCUGUGAAGACAAAAGAGUUUUAGAGCUAGAAAU | 863 |
| 49 | (CITA) | | AAAAGA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | GCCCUGGAGGGACA | 697 | GCCCUGGAGGGACAUCACCAGUUUUAGAGCUAGAAAU | 864 |
| 50 | (CITA) | | UCACCA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | CUGGGCAGGCUGAC | 698 | CUGGGCAGGCUGACACAUUGGUUUUAGAGCUAGAAAU | 865 |
| 51 | (CITA) | | ACAUUG | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | AUCUCCAGCCUCAG | 699 | AUCUCCAGCCUCAGUGAGAAGUUUUAGAGCUAGAAAU | 866 |
| 52 | (CITA) | | UGAGAA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | ACUGAGGCUGGAGA | 700 | ACUGAGGCUGGAGAUCACAGGUUUUAGAGCUAGAAAU | 867 |
| 53 | (CITA) | | UCACAG | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | ACGCUCACCUGAUG | 701 | ACGCUCACCUGAUGUCCAAGGUUUUAGAGCUAGAAAU | 868 |
| 54 | (CITA) | | UCCAAG | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | ACGCUCACCUGAUG | 702 | ACGCUCACCUGAUGUCCAAGGUUUUAGAGCUAGAAAU | 869 |
| 54 | (CITA) | | UCCAAG | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | CCUAGGGAUAUGUG | 703 | CCUAGGGAUAUGUGGGCCACGUUUUAGAGCUAGAAAU | 870 |
| 55 | (CITA) | | GGCCAC | | AGCAAGUUAAAAUAAGGCUAGUCCGUUAUCAACUUGA | |
| | | | | | AAAAGUGGCACCGAGUCGGUGCUUUU | |
| TSBT×36 | NLRC5 | CBE | CUGAGGCUGCAAAG | 704 | CUGAGGCUGCAAAGGGGCAAGUUUUAGAGCUAGAAAU | 871 |
| 56 | (CITA) | | GGGCAA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | GACAAUCUGUGAAA | 705 | GACAAUCUGUGAAACAAAGCGUUUUAGAGCUAGAAAU | 872 |
| 57 | (CITA) | | CAAAGC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | CCUUUUCAGGCUGA | 706 | CCUUUUCAGGCUGAGCCAGAGUUUUAGAGCUAGAAAU | 873 |
| 58 | (CITA) | | GCCAGA | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | CUUUUCAGGCUGAG | 707 | CUUUUCAGGCUGAGCCAGACGUUUUAGAGCUAGAAAU | 874 |
| 59 | (CITA) | | CCAGAC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | GACCUGGAUCAGAC | 708 | GACCUGGAUCAGACAGGACCGUUUUAGAGCUAGAAAU | 875 |
| 60 | (CITA) | | AGGACC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | CAACUUACUCCACC | 709 | CAACUUACUCCACCUGGCUGGUUUUAGAGCUAGAAAU | 876 |
| 61 | (CITA) | | UGGCUG | | AGCAAGUUAAAAUAAGGCUAGUCCGUUAUCAACUUGA | |
| | | | | | AAAAGUGGCACCGAGUCGGUGCUUUU | |
| TSBT×36 | NLRC5 | ABE | CAACUUACUCCACC | 710 | CAACUUACUCCACCUGGCUGGUUUUAGAGCUAGAAAU | 877 |
| 61 | (CITA) | | UGGCUG | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | CBE | CUUACUUCACUGAG | 711 | CUUACUUCACUGAGGCCUCCGUUUUAGAGCUAGAAAU | 878 |
| 62 | (CITA) | | GCCUCC | | AGCAAGUUAAAAUAAGGCUAGUCCGUUAUCAACUUGA | |
| | | | | | AAAAGUGGCACCGAGUCGGUGCUUUU | |
| TSBTx36 | NLRC5 | ABE | CUUACUUCACUGAG | 712 | CUUACUUCACUGAGGCCUCCGUUUUAGAGCUAGAAAU | 879 |
| 62 | (CITA) | | GCCUCC | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | GCUCACCCAAAGCC | 713 | GCUCACCCAAAGCCACAGCGGUUUUAGAGCUAGAAAU | 880 |
| 63 | (Tapasin) | | ACAGCG | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | ABE | GCUCACCCAAAGCC | 714 | GCUCACCCAAAGCCACAGCGGUUUUAGAGCUAGAAAU | 881 |
| 63 | (Tapasin) | | ACAGCG | | AGCAAGUUAAAAUAAGGCUAGUCCGUUAUCAACUUGA | |
| | | | | | AAAAGUGGCACCGAGUCGGUGCUUUU | |
| TSBT×36 | TAPBP | CBE | GCUCUUACCUGCUA | 715 | GCUCUUACCUGCUACCUCCAGUUUUAGAGCUAGAAAU | 882 |
| 64 | (Tapasin) | | CCUCCA | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | ABE | GCUCUUACCUGCUA | 716 | GCUCUUACCUGCUACCUCCAGUUUUAGAGCUAGAAAU | 883 |
| 64 | (Tapasin) | | CCUCCA | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | AAAGACCUGGCAGG | 717 | AAAGACCUGGCAGGCAGAAGGUUUUAGAGCUAGAAAU | 884 |
| 65 | (Tapasin) | | CAGAAG | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | GAACCAACACUCGA | 718 | GAACCAACACUCGAUCACCGGUUUUAGAGCUAGAAAU | 885 |
| 66 | (Tapasin) | | UCACCG | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | GCUGGCCCAUUUCG | 719 | GCUGGCCCAUUUCGCAGAGGGUUUUAGAGCUAGAAAU | 886 |
| 67 | (Tapasin) | | CAGAGG | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | CUGGCCCAUUUCGC | 720 | CUGGCCCAUUUCGCAGAGGCGUUUUAGAGCUAGAAAU | 887 |
| 68 | (Tapasin) | | AGAGGC | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | ACCACAGCCAGAGC | 721 | ACCACAGCCAGAGCCUCAGCGUUUUAGAGCUAGAAAU | 888 |
| 69 | (Tapasin) | | CUCAGC | | AGCAAGUUAAAAUAAGGCUAGUCCGUUAUCAACUUGA | |
| | | | | | AAAAGUGGCACCGAGUCGGUGCUUUU | |
| TSBT×36 | TAPBP | CBE | ACUGGGACAAGAUG | 722 | ACUGGGACAAGAUGCUCUGCGUUUUAGAGCUAGAAAU | 889 |
| 70 | (Tapasin) | | CUCUGC | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | CGUCGCCACUCUAG | 723 | CGUCGCCACUCUAGCCCAAAGUUUUAGAGCUAGAAAU | 890 |
| 71 | (Tapasin) | | CCCAAA | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | GCGACGCCAGCACC | 724 | GCGACGCCAGCACCUGGGUAGUUUUAGAGCUAGAAAU | 891 |
| 72 | (Tapasin) | | UGGGUA | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | CGACGCCAGCACCU | 725 | CGACGCCAGCACCUGGGUAAGUUUUAGAGCUAGAAAU | 892 |
| 73 | (Tapasin) | | GGGUAA | | AGCAAGUUAAAAUAAGGCUAGUCCGUUAUCAACUUGA | |
| | | | | | AAAAGUGGCACCGAGUCGGUGCUUUU | |
| TSBT×36 | TAPBP | CBE | AUCCCAAGCAGCAA | 726 | AUCCCAAGCAGCAAAUGCCAGUUUUAGAGCUAGAAAU | 893 |
| 74 | (Tapasin) | | AUGCCA | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | ACAGUUCAACCCUU | 727 | ACAGUUCAACCCUUUCAGGAGUUUUAGAGCUAGAAAU | 894 |
| 75 | (Tapasin) | | UCAGGA | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | CUUUCAGGAGGGCA | 728 | CUUUCAGGAGGGCACCUAUCGUUUUAGAGCUAGAAAU | 895 |
| 76 | (Tapasin) | | CCUAUC | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | CCUGCAAGGACAGG | 729 | CCUGCAAGGACAGGUCACCCGUUUUAGAGCUAGAAAU | 896 |
| 77 | (Tapasin) | | UCACCC | | AGCAAGUUAAAAUAAGGCUAGUCCGUUAUCAACUUGA | |
| | | | | | AAAAGUGGCACCGAGUCGGUGCUUUU | |
| TSBTx36 | TAPBP | CBE | CGAGAGCCACCUCU | 730 | CGAGAGCCACCUCUGCCCCUGUUUUAGAGCUAGAAAU | 897 |
| 78 | (Tapasin) | | GCCCCU | | AGCAAGUUAAAAUAAGGCUAGUCCGUUAUCAACUUGA | |
| | | | | | AAAAGUGGCACCGAGUCGGUGCUUUU | |
| TSBTx36 | CD155 | ABE | AACUGGCAUGGCCC | 731 | AACUGGCAUGGCCCGAGCCAGUUUUAGAGCUAGAAAU | 898 |
| 79 | | | GAGCCA | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | CACUCACCGGUUCC | 732 | CACUCACCGGUUCCUGGGGGGUUUUAGAGCUAGAAAU | 899 |
| 80 | | | UGGGGG | | AGCAAGUUAAAAUAAGGCUAGUCCGUUAUCAACUUGA | |
| | | | | | AAAAGUGGCACCGAGUCGGUGCUUUU | |
| TSBTx36 | CD155 | ABE | CACUCACCGGUUCC | 733 | CACUCACCGGUUCCUGGGGGGUUUUAGAGCUAGAAAU | 900 |
| 80 | | | UGGGGG | | AGCAAGUUAAAAUAAGGCUAGUCCGUUAUCAACUUGA | |
| | | | | | AAAAGUGGCACCGAGUCGGUGCUUUU | |
| TSBT×36 | CD155 | ABE | CGCAGGGGACGUCG | 734 | CGCAGGGGACGUCGUCGUGCGUUUUAGAGCUAGAAAU | 901 |
| 81 | | | UCGUGC | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | CACACUCACAGUAC | 735 | CACACUCACAGUACACGGUGGUUUUAGAGCUAGAAAU | 902 |
| 82 | | | ACGGUG | | AGCAAGUUAAAAUAAGGCUAGUCCGUUAUCAACUUGA | |
| | | | | | AAAAGUGGCACCGAGUCGGUGCUUUU | |
| TSBTx36 | CD155 | ABE | CACACUCACAGUAC | 736 | CACACUCACAGUACACGGUGGUUUUAGAGCUAGAAAU | 903 |
| 82 | | | ACGGUG | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | UGGUCCUGGGAGAG | 737 | UGGUCCUGGGAGAGACAGAAGUUUUAGAGCUAGAAAU | 904 |
| 83 | | | ACAGAA | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | UUCCUCACCUUUGA | 738 | UUCCUCACCUUUGACCUGGAGUUUUAGAGCUAGAAAU | 905 |
| 84 | | | CCUGGA | | AGCAAGUUAAAAUAAGGCUAGUCCGUUAUCAACUUGA | |
| | | | | | AAAAGUGGCACCGAGUCGGUGCUUUU | |
| TSBTx36 | CD155 | ABE | UUCCUCACCUUUGA | 739 | UUCCUCACCUUUGACCUGGAGUUUUAGAGCUAGAAAU | 906 |
| 84 | | | CCUGGA | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | CCUACAGGUGCCCA | 740 | CCUACAGGUGCCCAACAUGGGUUUUAGAGCUAGAAAU | 907 |
| 85 | | | ACAUGG | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | UGUCACAGCUGACU | 741 | UGUCACAGCUGACUUGGGCGGUUUUAGAGCUAGAAAU | 908 |
| 86 | | | UGGGCG | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | AACGCAGGGCCCCA | 742 | AACGCAGGGCCCCAGCUAUUGUUUUAGAGCUAGAAAU | 909 |
| 87 | | | GCUAUU | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | CCCGCAGGGCAGCA | 743 | CCCGCAGGGCAGCAGGAGCGGUUUUAGAGCUAGAAAU | 910 |
| 88 | | | GGAGCG | | AGCAAGUUAAAAUAAGGCUAGUCCGUUAUCAACUUGA | |
| | | | | | AAAAGUGGCACCGAGUCGGUGCUUUU | |
| TSBTx36 | CD155 | CBE | GAGUGCCAGGUGAU | 744 | GAGUGCCAGGUGAUUUGGGCGUUUUAGAGCUAGAAAU | 911 |
| 89 | | | UUGGGC | | AGCAAGUUAAAAUAAGGCUAGUCCGUUAUCAACUUGA | |
| | | | | | AAAAGUGGCACCGAGUCGGUGCUUUU | |
| TSBT×36 | MICA | ABE | CGGGGCCAUGGGGC | 745 | CGGGGCCAUGGGGCUGGGCCGUUUUAGAGCUAGAAAU | 912 |
| 90 | | | UGGGCC | | AGCAAGUUAAAAUAAGGCUAGUCCGUUAUCAACUUGA | |
| | | | | | AAAAGUGGCACCGAGUCGGUGCUUUU | |
| TSBTx36 | MICA | CBE | UCACCAGCAGCAGC | 746 | UCACCAGCAGCAGCUCCCGGGUUUUAGAGCUAGAAAU | 913 |
| 91 | | | UCCCGG | | AGCAAGUUAAAAUAAGGCUAGUCCGUUAUCAACUUGA | |
| | | | | | AAAAGUGGCACCGAGUCGGUGCUUUU | |
| TSBTx36 | MICA | ABE | UCACCAGCAGCAGC | 747 | UCACCAGCAGCAGCUCCCGGGUUUUAGAGCUAGAAAU | 914 |
| 91 | | | UCCCGG | | AGCAAGUUAAAAUAAGGCUAGUCCGUUAUCAACUUGA | |
| | | | | | AAAAGUGGCACCGAGUCGGUGCUUUU | |
| TSBTx36 | MICA | CBE | CACUCACCAGCAGC | 748 | CACUCACCAGCAGCAGCUCCGUUUUAGAGCUAGAAAU | 915 |
| 92 | | | AGCUCC | | AGCAAGUUAAAAUAAGGCUAGUCCGUUAUCAACUUGA | |
| | | | | | AAAAGUGGCACCGAGUCGGUGCUUUU | |
| TSBTx36 | MICA | ABE | CACUCACCAGCAGC | 749 | CACUCACCAGCAGCAGCUCCGUUUUAGAGCUAGAAAU | 916 |
| 92 | | | AGCUCC | | AGCAAGUUAAAAUAAGGCUAGUCCGUUAUCAACUUGA | |
| | | | | | AAAAGUGGCACCGAGUCGGUGCUUUU | |
| TSBTx36 | MICA | ABE | GGCAGGCUUGCAUU | 750 | GGCAGGCUUGCAUUCCCUCCGUUUUAGAGCUAGAAAU | 917 |
| 93 | | | CCCUCC | | AGCAAGUUAAAAUAAGGCUAGUCCGUUAUCAACUUGA | |
| | | | | | AAAAGUGGCACCGAGUCGGUGCUUUU | |
| TSBTx36 | MICB | ABE | GGCAGGCUUGCAUU | 751 | GGCAGGCUUGCAUUCCCUCCGUUUUAGAGCUAGAAAU | 918 |
| 93 | | | CCCUCC | | AGCAAGUUAAAAUAAGGCUAGUCCGUUAUCAACUUGA | |
| | | | | | AAAAGUGGCACCGAGUCGGUGCUUUU | |
| TSBTx36 | MICA | CBE | CGUCGGUACCUGUU | 752 | CGUCGGUACCUGUUCUCCUCGUUUUAGAGCUAGAAAU | 919 |
| 94 | | | CUCCUC | | AGCAAGUUAAAAUAAGGCUAGUCCGUUAUCAACUUGA | |
| | | | | | AAAAGUGGCACCGAGUCGGUGCUUUU | |
| TSBTx36 | MICA | ABE | CGUCGGUACCUGUU | 753 | CGUCGGUACCUGUUCUCCUCGUUUUAGAGCUAGAAAU | 920 |
| 94 | | | CUCCUC | | AGCAAGUUAAAAUAAGGCUAGUCCGUUAUCAACUUGA | |
| | | | | | AAAAGUGGCACCGAGUCGGUGCUUUU | |
| TSBT×36 | MICA | ABE | UUCUUCUCCAGUGC | 754 | UUCUUCUCCAGUGCCCCCCAGUUUUAGAGCUAGAAAU | 921 |
| 95 | | | CCCCCA | | AGCAAGUUAAAAUAAGGCUAGUCCGUUAUCAACUUGA | |
| | | | | | AAAAGUGGCACCGAGUCGGUGCUUUU | |
| TSBTx36 | MICA | CBE | UAGGCUCACCAGAG | 755 | UAGGCUCACCAGAGGGCACAGUUUUAGAGCUAGAAAU | 922 |
| 96 | | | GGCACA | | AGCAAGUUAAAAUAAGGCUAGUCCGUUAUCAACUUGA | |
| | | | | | AAAAGUGGCACCGAGUCGGUGCUUUU | |
| TSBTx36 | MICA | ABE | UAGGCUCACCAGAG | 756 | UAGGCUCACCAGAGGGCACAGUUUUAGAGCUAGAAAU | 923 |
| 96 | | | GGCACA | | AGCAAGUUAAAAUAAGGCUAGUCCGUUAUCAACUUGA | |
| | | | | | AAAAGUGGCACCGAGUCGGUGCUUUU | |
| TSBTx36 | MICA | CBE | CUGUCCAUUCCUCA | 757 | CUGUCCAUUCCUCAGUCUCCGUUUUAGAGCUAGAAAU | 924 |
| 97 | | | GUCUCC | | AGCAAGUUAAAAUAAGGCUAGUCCGUUAUCAACUUGA | |
| | | | | | AAAAGUGGCACCGAGUCGGUGCUUUU | |
| TSBTx36 | MICA | CBE | UGUCCCAUGUCUUA | 758 | UGUCCCAUGUCUUAUUUCCCGUUUUAGAGCUAGAAAU | 925 |
| 98 | | | UUUCCC | | AGCAAGUUAAAAUAAGGCUAGUCCGUUAUCAACUUGA | |
| | | | | | AAAAGUGGCACCGAGUCGGUGCUUUU | |
| TSBTx36 | MICA | CBE | CCCGCCUUCUAAAU | 759 | CCCGCCUUCUAAAUCUCCCCGUUUUAGAGCUAGAAAU | 926 |
| 99 | | | CUCCCC | | AGCAAGUUAAAAUAAGGCUAGUCCGUUAUCAACUUGA | |
| | | | | | AAAAGUGGCACCGAGUCGGUGCUUUU | |
| TSBTx37 | MICA | ABE | GGAAUUUUCUCUUC | 760 | GGAAUUUUCUCUUCCAAGCGGUUUUAGAGCUAGAAAU | 927 |
| 00 | | | CAAGCG | | AGCAAGUUAAAAUAAGGCUAGUCCGUUAUCAACUUGA | |
| | | | | | AAAAGUGGCACCGAGUCGGUGCUUUU | |
| TSBTx37 | MICA | ABE | AAGAGAAAAUUCCA | 761 | AAGAGAAAAUUCCAGUGGGCGUUUUAGAGCUAGAAAU | 928 |
| 01 | | | GUGGGC | | AGCAAGUUAAAAUAAGGCUAGUCCGUUAUCAACUUGA | |
| | | | | | AAAAGUGGCACCGAGUCGGUGCUUUU | |
| TSBTx37 | MICA | CBE | GGAGAGGGCGGGGC | 762 | GGAGAGGGGGGGGCCACGCUGUUUUAGAGCUAGAAAU | 929 |
| 02 | | | CACGCU | | AGCAAGUUAAAAUAAGGCUAGUCCGUUAUCAACUUGA | |
| | | | | | AAAAGUGGCACCGAGUCGGUGCUUUU | |
| TSBTx37 | MICA | CBE | CCGCCCUCUCCGCU | 763 | CCGCCCUCUCCGCUCGUGAUGUUUUAGAGCUAGAAAU | 930 |
| 03 | | | CGUGAU | | AGCAAGUUAAAAUAAGGCUAGUCCGUUAUCAACUUGA | |
| | | | | | AAAAGUGGCACCGAGUCGGUGCUUUU | |
| TSBTx37 | MICA | CBE | AGGGCCAAUCACGA | 764 | AGGGCCAAUCACGAGCGGAGGUUUUAGAGCUAGAAAU | 931 |
| 04 | | | GCGGAG | | AGCAAGUUAAAAUAAGGCUAGUCCGUUAUCAACUUGA | |
| | | | | | AAAAGUGGCACCGAGUCGGUGCUUUU | |
| TSBTx37 | MICA | ABE | UCGUGAUUGGCCCU | 765 | UCGUGAUUGGCCCUAAGUUCGUUUUAGAGCUAGAAAU | 932 |
| 05 | | | AAGUUC | | AGCAAGUUAAAAUAAGGCUAGUCCGUUAUCAACUUGA | |
| | | | | | AAAAGUGGCACCGAGUCGGUGCUUUU | |
| TSBTx37 | MICB | ABE | AGGGGCCAUGGGGC | 766 | AGGGGCCAUGGGGCUGGGCCGUUUUAGAGCUAGAAAU | 933 |
| 06 | | | UGGGCC | | AGCAAGUUAAAAUAAGGCUAGUCCGUUAUCAACUUGA | |
| | | | | | AAAAGUGGCACCGAGUCGGUGCUUUU | |
| TSBTx37 | MICB | CBE | ACUCACCAGCGGCG | 767 | ACUCACCAGCGGCGGCUGCCGUUUUAGAGCUAGAAAU | 934 |
| 07 | | | GCUGCC | | AGCAAGUUAAAAUAAGGCUAGUCCGUUAUCAACUUGA | |
| | | | | | AAAAGUGGCACCGAGUCGGUGCUUUU | |
| TSBTx37 | MICB | ABE | ACUCACCAGCGGCG | 768 | ACUCACCAGCGGCGGCUGCCGUUUUAGAGCUAGAAAU | 935 |
| 07 | | | GCUGCC | | AGCAAGUUAAAAUAAGGCUAGUCCGUUAUCAACUUGA | |
| | | | | | AAAAGUGGCACCGAGUCGGUGCUUUU | |
| TSBTx37 | MICB | CBE | CUCACCAGCGGCGG | 769 | CUCACCAGCGGCGGCUGCCGGUUUUAGAGCUAGAAAU | 936 |
| 08 | | | CUGCCG | | AGCAAGUUAAAAUAAGGCUAGUCCGUUAUCAACUUGA | |
| | | | | | AAAAGUGGCACCGAGUCGGUGCUUUU | |
| TSBTx37 | MICB | ABE | CUCACCAGCGGCGG | 770 | CUCACCAGCGGCGGCUGCCGGUUUUAGAGCUAGAAAU | 937 |
| 08 | | | CUGCCG | | AGCAAGUUAAAAUAAGGCUAGUCCGUUAUCAACUUGA | |
| | | | | | AAAAGUGGCACCGAGUCGGUGCUUUU | |
| TSBTx37 | MICB | CBE | CACUCACCAGCGGC | 771 | CACUCACCAGCGGCGGCUGCGUUUUAGAGCUAGAAAU | 938 |
| 09 | | | GGCUGC | | AGCAAGUUAAAAUAAGGCUAGUCCGUUAUCAACUUGA | |
| | | | | | AAAAGUGGCACCGAGUCGGUGCUUUU | |
| TSBTx37 | MICB | ABE | CACUCACCAGCGGC | 772 | CACUCACCAGCGGCGGCUGCGUUUUAGAGCUAGAAAU | 939 |
| 09 | | | GGCUGC | | AGCAAGUUAAAAUAAGGCUAGUCCGUUAUCAACUUGA | |
| | | | | | AAAAGUGGCACCGAGUCGGUGCUUUU | |
| TSBTx37 | MICB | CBE | CUACAGCGAUAUCU | 773 | CUACAGCGAUAUCUGAAAUCGUUUUAGAGCUAGAAAU | 940 |
| 10 | | | GAAAUC | | AGCAAGUUAAAAUAAGGCUAGUCCGUUAUCAACUUGA | |
| | | | | | AAAAGUGGCACCGAGUCGGUGCUUUU | |
| TSBTx37 | MICB | CBE | GUCGGUACCUGUUC | 774 | GUCGGUACCUGUUCUCCUGAGUUUUAGAGCUAGAAAU | 941 |
| 11 | | | UCCUGA | | AGCAAGUUAAAAUAAGGCUAGUCCGUUAUCAACUUGA | |
| | | | | | AAAAGUGGCACCGAGUCGGUGCUUUU | |
| TSBTx37 | MICB | ABE | GUCGGUACCUGUUC | 775 | GUCGGUACCUGUUCUCCUGAGUUUUAGAGCUAGAAAU | 942 |
| 11 | | | UCCUGA | | AGCAAGUUAAAAUAAGGCUAGUCCGUUAUCAACUUGA | |
| | | | | | AAAAGUGGCACCGAGUCGGUGCUUUU | |
| TSBTx37 | MICB | CBE | CAGGCUCACCAGAG | 776 | CAGGCUCACCAGAGGGCACAGUUUUAGAGCUAGAAAU | 943 |
| 12 | | | GGCACA | | AGCAAGUUAAAAUAAGGCUAGUCCGUUAUCAACUUGA | |
| | | | | | AAAAGUGGCACCGAGUCGGUGCUUUU | |
| TSBTx37 | MICB | ABE | CAGGCUCACCAGAG | 777 | CAGGCUCACCAGAGGGCACAGUUUUAGAGCUAGAAAU | 944 |
| 12 | | | GGCACA | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | CBE | GAGGCGCAUGGCGG | 778 | GAGGCGCAUGGCGGCGAGGUGUUUUAGAGCUAGAAAU | 945 |
| 24 | (ERp57) | | CGAGGU | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | ABE | GAGGCGCAUGGCGG | 779 | GAGGCGCAUGGCGGCGAGGUGUUUUAGAGCUAGAAAU | 946 |
| 24 | (ERp57) | | CGAGGU | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | CBE | CGCAUGGCGGCGAG | 780 | CGCAUGGCGGCGAGGUGGGGGUUUUAGAGCUAGAAAU | 947 |
| 25 | (ERp57) | | GUGGGG | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | ABE | CGCAUGGCGGCGAG | 781 | CGCAUGGCGGCGAGGUGGGGGUUUUAGAGCUAGAAAU | 948 |
| 25 | (ERp57) | | GUGGGG | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | CBE | AGGCGCAUGGCGGC | 782 | AGGCGCAUGGCGGCGAGGUGGUUUUAGAGCUAGAAAU | 949 |
| 26 | (ERp57) | | GAGGUG | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | ABE | AGGCGCAUGGCGGC | 783 | AGGCGCAUGGCGGCGAGGUGGUUUUAGAGCUAGAAAU | 950 |
| 26 | (ERp57) | | GAGGUG | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | ABE | UUCCCACAGGGUAA | 784 | UUCCCACAGGGUAAUGAUGGGUUUUAGAGCUAGAAAU | 951 |
| 27 | (ERp57) | | UGAUGG | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | CBE | ACGCCUACAAUUGG | 785 | ACGCCUACAAUUGGAAAACAGUUUUAGAGCUAGAAAU | 952 |
| 28 | (ERp57) | | AAAACA | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | CBE | CGCCUACAAUUGGA | 786 | CGCCUACAAUUGGAAAACAAGUUUUAGAGCUAGAAAU | 953 |
| 29 | (ERp57) | | AAACAA | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | ABE | CAAUUGUAGGCGUG | 787 | CAAUUGUAGGCGUGAUGGGAGUUUUAGAGCUAGAAAU | 954 |
| 30 | (ERp57) | | AUGGGA | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | CBE | UGCUGAGCUGUUAA | 788 | UGCUGAGCUGUUAAUAAAACGUUUUAGAGCUAGAAAU | 955 |
| 31 | (ERp57) | | UAAAAC | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | CBE | ACUUACCCUCUGAC | 789 | ACUUACCCUCUGACUUCAUAGUUUUAGAGCUAGAAAU | 956 |
| 32 | (ERp57) | | UUCAUA | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | ABE | ACUUACCCUCUGAC | 790 | ACUUACCCUCUGACUUCAUAGUUUUAGAGCUAGAAAU | 957 |
| 32 | (ERp57) | | UUCAUA | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | CBE | UCACUUACUUCAUA | 791 | UCACUUACUUCAUAUUUCUUGUUUUAGAGCUAGAAAU | 958 |
| 33 | (ERp57) | | UUUCUU | | AGCAAGUUAAAAUAAGGCUAGUCCGUUAUCAACUUGA | |
| | | | | | AAAAGUGGCACCGAGUCGGUGCUUUU | |
| TSBTx37 | PDIA3 | ABE | UCACUUACUUCAUA | 792 | UCACUUACUUCAUAUUUCUUGUUUUAGAGCUAGAAAU | 959 |
| 33 | (ERp57) | | UUUCUU | | AGCAAGUUAAAAUAAGGCUAGUCCGUUAUCAACUUGA | |
| | | | | | AAAAGUGGCACCGAGUCGGUGCUUUU | |
| TSBTx39 | TAP1 | Cas12b | CAUUCUUUACGGGC | 793 | GUUCUGUCUUUUGGUCAGGACAACCGUCUAGCUAUAA | 960 |
| 23 | | | CGCCUCAC | | GUGCUGCAGGGUGUGAGAAACUCCUAUUGCUGGACGA | |
| | | | | | UGUCUCUUACGAGGCAUUAGCACCAUUCUUUACGGGC | |
| | | | | | CGCCUCAC | |
| TSBTx39 | TAP1 | Cas12b | UUUACGGGCCGCCU | 794 | GUUCUGUCUUUUGGUCAGGACAACCGUCUAGCUAUAA | 961 |
| 24 | | | CACUGACU | | GUGCUGCAGGGUGUGAGAAACUCCUAUUGCUGGACGA | |
| | | | | | UGUCUCUUACGAGGCAUUAGCACUUUACGGGCCGCCU | |
| | | | | | CACUGACU | |
| TSBTx39 | TAP1 | Cas12b | UACAAGAUGGCUCA | 795 | GUUCUGUCUUUUGGUCAGGACAACCGUCUAGCUAUAA | 962 |
| 25 | | | GCCGAUAC | | GUGCUGCAGGGUGUGAGAAACUCCUAUUGCUGGACGA | |
| | | | | | UGUCUCUUACGAGGCAUUAGCACUACAAGAUGGCUCA | |
| | | | | | GCCGAUAC | |
| TSBTx39 | TAPI | Cas12b | UCACCAUAGCCAGG | 796 | GUUCUGUCUUUUGGUCAGGACAACCGUCUAGCUAUAA | 963 |
| 26 | | | UCUGGGGG | | GUGCUGCAGGGUGUGAGAAACUCCUAUUGCUGGACGA | |
| | | | | | UGUCUCUUACGAGGCAUUAGCACUCACCAUAGCCAGG | |
| | | | | | UCUGGGGG | |
| TSBTx39 | TAP1 | Cas12b | UCUGAGUGAGAAUC | 797 | GUUCUGUCUUUUGGUCAGGACAACCGUCUAGCUAUAA | 964 |
| 27 | | | UGAGCUUA | | GUGCUGCAGGGUGUGAGAAACUCCUAUUGCUGGACGA | |
| | | | | | UGUCUCUUACGAGGCAUUAGCACUCUGAGUGAGAAUC | |
| | | | | | UGAGCUUA | |
| TSBTx39 | TAP1 | Cas12b | UCACUCAGAGAAUC | 798 | GUUCUGUCUUUUGGUCAGGACAACCGUCUAGCUAUAA | 965 |
| 28 | | | ACUCAGGG | | GUGCUGCAGGGUGUGAGAAACUCCUAUUGCUGGACGA | |
| | | | | | UGUCUCUUACGAGGCAUUAGCACUCACUCAGAGAAUC | |
| | | | | | ACUCAGGG | |
| TSBTx39 | TAP1 | Cas12b | CUGUGGUACCUGGU | 799 | GUUCUGUCUUUUGGUCAGGACAACCGUCUAGCUAUAA | 966 |
| 29 | | | GCGAGGCC | | GUGCUGCAGGGUGUGAGAAACUCCUAUUGCUGGACGA | |
| | | | | | UGUCUCUUACGAGGCAUUAGCACCUGUGGUACCUGGU | |
| | | | | | GCGAGGCC | |
| TSBTx39 | TAP1 | Cas12b | UCCCACCUUCUUGG | 800 | GUUCUGUCUUUUGGUCAGGACAACCGUCUAGCUAUAA | 967 |
| 30 | | | GCAGAAGG | | GUGCUGCAGGGUGUGAGAAACUCCUAUUGCUGGACGA | |
| | | | | | UGUCUCUUACGAGGCAUUAGCACUCCCACCUUCUUGG | |
| | | | | | GCAGAAGG | |
| TSBTx39 | TAP1 | Cas12b | CCGCACCUGCACUU | 801 | GUUCUGUCUUUUGGUCAGGACAACCGUCUAGCUAUAA | 968 |
| 31 | | | CCAGCAAC | | GUGCUGCAGGGUGUGAGAAACUCCUAUUGCUGGACGA | |
| | | | | | UGUCUCUUACGAGGCAUUAGCACCCGCACCUGCACUU | |
| | | | | | CCAGCAAC | |
| TSBTx39 | TAP1 | Cas12b | AGGCUCUGUCGGCC | 802 | GUUCUGUCUUUUGGUCAGGACAACCGUCUAGCUAUAA | 969 |
| 32 | | | AUGCCUAC | | GUGCUGCAGGGUGUGAGAAACUCCUAUUGCUGGACGA | |
| | | | | | UGUCUCUUACGAGGCAUUAGCACAGGCUCUGUCGGCC | |
| | | | | | AUGCCUAC | |
| TSBTx39 | TAP2 | Cas12b | GUGGGGACACUGCU | 803 | GUUCUGUCUUUUGGUCAGGACAACCGUCUAGCUAUAA | 970 |
| 33 | | | GCUCCCGC | | GUGCUGCAGGGUGUGAGAAACUCCUAUUGCUGGACGA | |
| | | | | | UGUCUCUUACGAGGCAUUAGCACGUGGGGACACUGCU | |
| | | | | | GCUCCCGC | |
| TSBTx39 | TAP2 | Cas12b | AUCCCUCACUAUUC | 804 | GUUCUGUCUUUUGGUCAGGACAACCGUCUAGCUAUAA | 971 |
| 34 | | | UGGUCGUG | | GUGCUGCAGGGUGUGAGAAACUCCUAUUGCUGGACGA | |
| | | | | | UGUCUCUUACGAGGCAUUAGCACAUCCCUCACUAUUC | |
| | | | | | UGGUCGUG | |
| TSBTx39 | TAP2 | Cas12b | UGGUCGUGUGAUUG | 805 | GUUCUGUCUUUUGGUCAGGACAACCGUCUAGCUAUAA | 972 |
| 35 | | | ACAUCCUG | | GUGCUGCAGGGUGUGAGAAACUCCUAUUGCUGGACGA | |
| | | | | | UGUCUCUUACGAGGCAUUAGCACUGGUCGUGUGAUUG | |
| | | | | | ACAUCCUG | |
| TSBTx39 | TAP2 | Cas12b | UGACCCCCAUGCCU | 806 | GUUCUGUCUUUUGGUCAGGACAACCGUCUAGCUAUAA | 973 |
| 36 | | | UUGCCAGU | | GUGCUGCAGGGUGUGAGAAACUCCUAUUGCUGGACGA | |
| | | | | | UGUCUCUUACGAGGCAUUAGCACUGACCCCCAUGCCU | |
| | | | | | UUGCCAGU | |
| TSBTx39 | TAP2 | Cas12b | GAGACAUGGUGUAG | 807 | GUUCUGUCUUUUGGUCAGGACAACCGUCUAGCUAUAA | 974 |
| 37 | | | GUGAAGCA | | GUGCUGCAGGGUGUGAGAAACUCCUAUUGCUGGACGA | |
| | | | | | UGUCUCUUACGAGGCAUUAGCACGAGACAUGGUGUAG | |
| | | | | | GUGAAGCA | |
| TSBTx39 | TAP2 | Cas12b | AAAGGAAGCCAGUU | 808 | GUUCUGUCUUUUGGUCAGGACAACCGUCUAGCUAUAA | 975 |
| 38 | | | ACUCAUCA | | GUGCUGCAGGGUGUGAGAAACUCCUAUUGCUGGACGA | |
| | | | | | UGUCUCUUACGAGGCAUUAGCACAAAGGAAGCCAGUU | |
| | | | | | ACUCAUCA | |
| TSBTx39 | TAP2 | Cas12b | UGAAGGGCAUGUGC | 809 | GUUCUGUCUUUUGGUCAGGACAACCGUCUAGCUAUAA | 976 |
| 39 | | | AGCAGAGA | | GUGCUGCAGGGUGUGAGAAACUCCUAUUGCUGGACGA | |
| | | | | | UGUCUCUUACGAGGCAUUAGCACUGAAGGGCAUGUGC | |
| | | | | | AGCAGAGA | |
| TSBTx39 | TAP2 | Cas12b | UUCAAGGGCCUCUU | 810 | GUUCUGUCUUUUGGUCAGGACAACCGUCUAGCUAUAA | 977 |
| 40 | | | UAUAGCGA | | GUGCUGCAGGGUGUGAGAAACUCCUAUUGCUGGACGA | |
| | | | | | UGUCUCUUACGAGGCAUUAGCACUUCAAGGGCCUCUU | |
| | | | | | UAUAGCGA | |
| TSBTx39 | TAP2 | Cas12b | GGCUGUCGGUCCAU | 811 | GUUCUGUCUUUUGGUCAGGACAACCGUCUAGCUAUAA | 978 |
| 41 | | | GUAGGAGA | | GUGCUGCAGGGUGUGAGAAACUCCUAUUGCUGGACGA | |
| | | | | | UGUCUCUUACGAGGCAUUAGCACGGCUGUCGGUCCAU | |
| | | | | | GUAGGAGA | |
| TSBTx39 | TAP2 | Cas12b | CACAACCCCCUGCA | 812 | GUUCUGUCUUUUGGUCAGGACAACCGUCUAGCUAUAA | 979 |
| 42 | | | GAGUGGUG | | GUGCUGCAGGGUGUGAGAAACUCCUAUUGCUGGACGA | |
| | | | | | UGUCUCUUACGAGGCAUUAGCACCACAACCCCCUGCA | |
| | | | | | GAGUGGUG | |
| TSBTx41 | HLA-A | ABE, | UGACGGCCAUCCUC | 813 | UGACGGCCAUCCUCGGCGUCGUUUUAGAGCUAGAAAU | 980 |
| 86 (846) | | CBE | GGCGUC | | AGCAAGUUAAAAUAAGGCUAGUCCGUUAUCAACUUGA | |
| | | | | | AAAAGUGGCACCGAGUCGGUGCUUUU | |
| TSBTx41 | HLA-A | ABE, | GACGGCCAUCCUCG | 814 | GACGGCCAUCCUCGGCGUCUGUUUUAGAGCUAGAAAU | 981 |
| 87 (847) | | CBE | GCGUCU | | AGCAAGUUAAAAUAAGGCUAGUCCGUUAUCAACUUGA | |
| | | | | | AAAAGUGGCACCGAGUCGGUGCUUUU | |
| TSBTx41 | HLA-A | ABE, | ACGGCCAUCCUCGG | 815 | ACGGCCAUCCUCGGCGUCUGGUUUUAGAGCUAGAAAU | 982 |
| 88 (848) | | CBE | CGUCUG | | AGCAAGUUAAAAUAAGGCUAGUCCGUUAUCAACUUGA | |
| | | | | | AAAAGUGGCACCGAGUCGGUGCUUUU | |
| TSBTx41 | HLA-A | ABE, | CGCACUCACCCGCC | 816 | CGCACUCACCCGCCCAGGUCGUUUUAGAGCUAGAAAU | 983 |
| 89 (849) | | CBE | CAGGUC | | AGCAAGUUAAAAUAAGGCUAGUCCGUUAUCAACUUGA | |
| | | | | | AAAAGUGGCACCGAGUCGGUGCUUUU | |
| TSBTx41 | HLA-A | ABE, | GCACUCACCCGCCC | 817 | GCACUCACCCGCCCAGGUCUGUUUUAGAGCUAGAAAU | 984 |
| 90 (850) | | CBE | AGGUCU | | AGCAAGUUAAAAUAAGGCUAGUCCGUUAUCAACUUGA | |
| | | | | | AAAAGUGGCACCGAGUCGGUGCUUUU | |
| TSBTx41 | HLA-A | ABE | CCCAGGCUCGCACU | 818 | CCCAGGCUCGCACUCCAUGGUUUUAGAGCUAGAAAUA | 985 |
| 91 (851) | | | CCAUG | | GCAAGUUAAAAUAAGGCUAGUCCGUUAUCAACUUGAA | |
| | | | | | AAAGUGGCACCGAGUCGGUGCUUUU | |
| TSBTx41 | HLA-A, | ABE, | GUCACUCACCGGCC | 819 | GUCACUCACCGGCCUCGCUCGUUUUAGAGCUAGAAAU | 986 |
| 92 (852) | HLA-B, | CBE | UCGCUC | | AGCAAGUUAAAAUAAGGCUAGUCCGUUAUCAACUUGA | |
| | HLA-C | | | | AAAAGUGGCACCGAGUCGGUGCUUUU | |
| TSBTx41 | HLA-A, | ABE, | CCUUACCCCAUCUC | 820 | CCUUACCCCAUCUCAGGGUGGUUUUAGAGCUAGAAAU | 987 |
| 93 (853) | HLA-B, | CBE | AGGGUG | | AGCAAGUUAAAAUAAGGCUAGUCCGUUAUCAACUUGA | |
| | HLA-C | | | | AAAAGUGGCACCGAGUCGGUGCUUUU | |
| TSBTx41 | HLA-A, | ABE, | CUUACCCCAUCUCA | 821 | CUUACCCCAUCUCAGGGUGAGUUUUAGAGCUAGAAAU | 988 |
| 94 (854) | HLA-B, | CBE | GGGUGA | | AGCAAGUUAAAAUAAGGCUAGUCCGUUAUCAACUUGA | |
| | HLA-C | | | | AAAAGUGGCACCGAGUCGGUGCUUUU | |
| TSBTx41 | HLA-A | CBE | GGCUCUGGGAAAAG | 822 | GGCUCUGGGAAAAGAGGGGAGUUUUAGAGCUAGAAAU | 989 |
| 95 (855) | | | AGGGGA | | AGCAAGUUAAAAUAAGGCUAGUCCGUUAUCAACUUGA | |
| | | | | | AAAAGUGGCACCGAGUCGGUGCUUUU | |
| TSBTx41 | HLA-A | ABE | CUUCCCACAGAUAG | 823 | CUUCCCACAGAUAGAAAAGGGUUUUAGAGCUAGAAAU | 990 |
| 96 (856) | | | AAAAGG | | AGCAAGUUAAAAUAAGGCUAGUCCGUUAUCAACUUGA | |
| | | | | | AAAAGUGGCACCGAGUCGGUGCUUUU | |
| TSBT×41 | HLA-A | ABE | UUCCCACAGAUAGA | 824 | UUCCCACAGAUAGAAAAGGAGUUUUAGAGCUAGAAAU | 991 |
| 97 (857) | | | AAAGGA | | AGCAAGUUAAAAUAAGGCUAGUCCGUUAUCAACUUGA | |
| | | | | | AAAAGUGGCACCGAGUCGGUGCUUUU | |
| TSBTx41 | HLA-A | ABE | CCCAGGCAGUGACA | 825 | CCCAGGCAGUGACAGUGCCCGUUUUAGAGCUAGAAAU | 992 |
| 98 (858) | | | GUGCCC | | AGCAAGUUAAAAUAAGGCUAGUCCGUUAUCAACUUGA | |
| | | | | | AAAAGUGGCACCGAGUCGGUGCUUUU | |
| TSBTx41 | HLA-A | ABE | CCAGGCAGUGACAG | 826 | CCAGGCAGUGACAGUGCCCAGUUUUAGAGCUAGAAAU | 993 |
| 99 (859) | | | UGCCCA | | AGCAAGUUAAAAUAAGGCUAGUCCGUUAUCAACUUGA | |
| | | | | | AAAAGUGGCACCGAGUCGGUGCUUUU | |
| TSBTx42 | HLA-B, | ABE | GACCCGCAUCUCGG | 827 | GACCCGCAUCUCGGCGUCUGGUUUUAGAGCUAGAAAU | 994 |
| 00 (860) | HLA-C | | CGUCUG | | AGCAAGUUAAAAUAAGGCUAGUCCGUUAUCAACUUGA | |
| | | | | | AAAAGUGGCACCGAGUCGGUGCUUUU | |
| TSBTx42 | HLA-B | ABE, | GCACUCACCGGCCC | 828 | GCACUCACCGGCCCAGGUCUGUUUUAGAGCUAGAAAU | 995 |
| 01 (861) | | CBE | AGGUCU | | AGCAAGUUAAAAUAAGGCUAGUCCGUUAUCAACUUGA | |
| | | | | | AAAAGUGGCACCGAGUCGGUGCUUUU | |
| TSBTx42 | HLA-B | CBE | UGGGAGCCUGGGGG | 829 | UGGGAGCCUGGGGGCGAGGAGUUUUAGAGCUAGAAAU | 996 |
| 02 (862) | | | CGAGGA | | AGCAAGUUAAAAUAAGGCUAGUCCGUUAUCAACUUGA | |
| | | | | | AAAAGUGGCACCGAGUCGGUGCUUUU | |
| TSBTx42 | HLA-B | CBE | GGGAGCCUGGGGGC | 830 | GGGAGCCUGGGGGCGAGGAGGUUUUAGAGCUAGAAAU | 997 |
| 03 (863) | | | GAGGAG | | AGCAAGUUAAAAUAAGGCUAGUCCGUUAUCAACUUGA | |
| | | | | | AAAAGUGGCACCGAGUCGGUGCUUUU | |
| TSBTx42 | HLA-B, | ABE | CCCCAGGCUCCCAC | 831 | CCCCAGGCUCCCACUCCAUGGUUUUAGAGCUAGAAAU | 998 |
| 04 (864) | HLA-C | | UCCAUG | | AGCAAGUUAAAAUAAGGCUAGUCCGUUAUCAACUUGA | |
| | | | | | AAAAGUGGCACCGAGUCGGUGCUUUU | |
| TSBTx42 | HLA-B, | ABE, | UUACCCCAUCUCAG | 832 | UUACCCCAUCUCAGGGUGAGGUUUUAGAGCUAGAAAU | 999 |
| 05 (865) | HLA-C | CBE | GGUGAG | | AGCAAGUUAAAAUAAGGCUAGUCCGUUAUCAACUUGA | |
| | | | | | AAAAGUGGCACCGAGUCGGUGCUUUU | |
| TSBTx42 | HLA-B, | ABE | UUCCCACAGGUGGA | 833 | UUCCCACAGGUGGAAAAGGAGUUUUAGAGCUAGAAAU | 1000 |
| 06 (866) | HLA-C | | AAAGGA | | AGCAAGUUAAAAUAAGGCUAGUCCGUUAUCAACUUGA | |
| | | | | | AAAAGUGGCACCGAGUCGGUGCUUUU | |
| TSBTx42 | HLA-C | ABE, | UGACCCGCAUCUCG | 834 | UGACCCGCAUCUCGGCGUCUGUUUUAGAGCUAGAAAU | 1001 |
| 07 (867) | | CBE | GCGUCU | | AGCAAGUUAAAAUAAGGCUAGUCCGUUAUCAACUUGA | |
| | | | | | AAAAGUGGCACCGAGUCGGUGCUUUU | |
| TSBTx42 | HLA-C | ABE, | GCACUCACAGGCCC | 835 | GCACUCACAGGCCCAGGUCUGUUUUAGAGCUAGAAAU | 1002 |
| 08 (868) | | CBE | AGGUCU | | AGCAAGUUAAAAUAAGGCUAGUCCGUUAUCAACUUGA | |
| | | | | | AAAAGUGGCACCGAGUCGGUGCUUUU | |
| TSBTx42 | HLA-C | ABE, | CACAGGCCCAGGUC | 836 | CACAGGCCCAGGUCUCGGUCGUUUUAGAGCUAGAAAU | 1003 |
| 09 (869) | | CBE | UCGGUC | | AGCAAGUUAAAAUAAGGCUAGUCCGUUAUCAACUUGA | |
| | | | | | AAAAGUGGCACCGAGUCGGUGCUUUU | |
| TSBTx42 | HLA-C | CBE | AGACCCUGGCCCCG | 837 | AGACCCUGGCCCCGCCCCCGGUUUUAGAGCUAGAAAU | 1004 |
| 10 (870) | | | CCCCCG | | AGCAAGUUAAAAUAAGGCUAGUCCGUUAUCAACUUGA | |
| | | | | | AAAAGUGGCACCGAGUCGGUGCUUUU | |
| TSBTx42 | HLA-C | CBE | GGCUCUGGGAAAGG | 838 | GGCUCUGGGAAAGGAGGGGAGUUUUAGAGCUAGAAAU | 1005 |
| 11 (871) | | | AGGGGA | | AGCAAGUUAAAAUAAGGCUAGUCCGUUAUCAACUUGA | |
| | | | | | AAAAGUGGCACCGAGUCGGUGCUUUU | |
| TSBTx42 | HLA-C | ABE | CCCAGCCAGCAACA | 839 | CCCAGCCAGCAACAGUGCCCGUUUUAGAGCUAGAAAU | 1006 |
| 12 (872) | | | GUGCCC | | AGCAAGUUAAAAUAAGGCUAGUCCGUUAUCAACUUGA | |
| | | | | | AAAAGUGGCACCGAGUCGGUGCUUUU | |
| TSBTx41 | HLA-C | Cas12b | GGACCGGGAGACAC | 840 | GUUCUGUCUUUUGGUCAGGACAACCGUCUAGCUAUAA | 1007 |
| 67 (873) | | | AGAAGUAC | | GUGCUGCAGGGUGUGAGAAACUCCUAUUGCUGGACGA | |
| | | | | | UGUCUCUUACGAGGCAUUAGCACGGACCGGGAGACAC | |
| | | | | | AGAAGUAC | |
| TSBTx41 | HLA-C, | Cas12b | CAUCGCCCUGAACG | 841 | GUUCUGUCUUUUGGUCAGGACAACCGUCUAGCUAUAA | 1008 |
| 68 (874) | HLA-B, | | AGGACCUG | | GUGCUGCAGGGUGUGAGAAACUCCUAUUGCUGGACGA | |
| | HLA-A | | | | UGUCUCUUACGAGGCAUUAGCACCAUCGCCCUGAACG | |
| | | | | | AGGACCUG | |
| TSBTx41 | HLA-C | Cas12b | CAUCGCCCUGAACG | 842 | GUUCUGUCUUUUGGUCAGGACAACCGUCUAGCUAUAA | 1009 |
| 69 (875) | | | AGGAUCUG | | GUGCUGCAGGGUGUGAGAAACUCCUAUUGCUGGACGA | |
| | | | | | UGUCUCUUACGAGGCAUUAGCACCAUCGCCCUGAACG | |
| | | | | | AGGAUCUG | |
| TSBTx41 | HLA-B | Cas12b | UUGCUGGCCUGGCU | 843 | GUUCUGUCUUUUGGUCAGGACAACCGUCUAGCUAUAA | 1010 |
| 70 (876) | | | GUCCUAGC | | GUGCUGCAGGGUGUGAGAAACUCCUAUUGCUGGACGA | |
| | | | | | UGUCUCUUACGAGGCAUUAGCACUUGCUGGCCUGGCU | |
| | | | | | GUCCUAGC | |
| TSBTx41 | HLA-A | Cas12b | CAUCGCCCUGAACG | 844 | GUUCUGUCUUUUGGUCAGGACAACCGUCUAGCUAUAA | 1011 |
| 68 (877) | | | AGGACCUG | | GUGCUGCAGGGUGUGAGAAACUCCUAUUGCUGGACGA | |
| | | | | | UGUCUCUUACGAGGCAUUAGCACCAUCGCCCUGAACG | |
| | | | | | AGGACCUG | |
| TSBTx42 | Nectin-2 | ABE/CB | CGGGCCAUGGAGGG | 1050 | CGGGCCAUGGAGGGCCCGGGGUUUUAGAGCUAGAAAU | 1065 |
| 13 | | E/CABE | CCCGGG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | ABE/CB | GGGCCAUGGAGGGC | 1051 | GGGCCAUGGAGGGCCCGGGAGUUUUAGAGCUAGAAAU | 1066 |
| 14 | | E/CABE | CCGGGA | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | ABE/CB | GGCCAUGGAGGGCC | 1052 | GGCCAUGGAGGGCCCGGGAGGUUUUAGAGCUAGAAAU | 1067 |
| 15 | | E/CABE | CGGGAG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | ABE/CB | CUCACCUAUGACUC | 1053 | CUCACCUAUGACUCUGAGCCGUUUUAGAGCUAGAAAU | 1068 |
| 16 | | E/CABE | UGAGCC | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | CBE | UUGGCUGGGGAGAG | 1054 | UUGGCUGGGGAGAGGAGGAGGUUUUAGAGCUAGAAAU | 1069 |
| 17 | | | GAGGAG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | CBE | UGGCUGGGGAGAGG | 1055 | UGGCUGGGGAGAGGAGGAGAGUUUUAGAGCUAGAAAU | 1070 |
| 18 | | | AGGAGA | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | ABE/CB | UACACUCACAGCGU | 1056 | UACACUCACAGCGUACAGAGGUUUUAGAGCUAGAAAU | 1071 |
| 19 | | E/CABE | ACAGAG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | ABE/CB | ACACUCACAGCGUA | 1057 | ACACUCACAGCGUACAGAGAGUUUUAGAGCUAGAAAU | 1072 |
| 20 | | E/CABE | CAGAGA | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | CBE | GGAGGGUCUGGGGG | 1058 | GGAGGGUCUGGGGGAAAUGGGUUUUAGAGCUAGAAAU | 1073 |
| 21 | | | AAAUGG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | CBE | GAGGGUCUGGGGGA | 1059 | GAGGGUCUGGGGGAAAUGGGGUUUUAGAGCUAGAAAU | 1074 |
| 22 | | | AAUGGG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | CBE | CAGGCUGUGGGGUA | 1060 | CAGGCUGUGGGGUAGGAGGUGUUUUAGAGCUAGAAAU | 1075 |
| 23 | | | GGAGGU | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | CBE | GGGCUGUAGUGGGA | 1061 | GGGCUGUAGUGGGAGAGGGGGUUUUAGAGCUAGAAAU | 1076 |
| 24 | | | GAGGGG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | CBE | GGAGGGCUGUAGUG | 1062 | GGAGGGCUGUAGUGGGAGAGGUUUUAGAGCUAGAAAU | 1077 |
| 25 | | | GGAGAG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | ABE/CB | UCCUACCUGCUCAG | 1063 | UCCUACCUGCUCAGUGCAUGGUUUUAGAGCUAGAAAU | 1078 |
| 26 | | E/CABE | UGCAUG | | AGCAAGUUAAAAUAAGGCUAGUCCGUUAUCAACUUGA | |
| | | | | | AAAAGUGGCACCGAGUCGGUGCUUUU | |
| TSBTx42 | Nectin-2 | CBE | CAUUUCCUGGAGGU | 1064 | CAUUUCCUGGAGGUAGAGAGGUUUUAGAGCUAGAAAU | 1079 |
| 27 | | | AGAGAG | | AGCAAGUUAAAAUAAGGCUAGUCCGUUAUCAACUUGA | |
| | | | | | AAAAGUGGCACCGAGUCGGUGCUUUU | |
| tap1_93 | TAP1 | CBE | ACCGCCCAGACCCG | 1121 | ACCGCCCAGACCCGGAGCAGGUUUUAGAGCUAGAAAU | 1148 |
| | | | GAGCAG | | AGCAAGUUAAAAUAAGGCUAGUCCGUUAUCAACUUGA | |
| | | | | | AAAAGUGGCACCGAGUCGGUGCUUUU | |
| tapbp_53 | TAPBP | CBE | GCGGGGUCUGAGUG | 1122 | GCGGGGUCUGAGUGUAGAGAGUUUUAGAGCUAGAAAU | 1149 |
| | | | UAGAGA | | AGCAAGUUAAAAUAAGGCUAGUCCGUUAUCAACUUGA | |
| | | | | | AAAAGUGGCACCGAGUCGGUGCUUUU | |
| tap2_5 | TAP2 | CBE | GAGGUCCAGGGUCU | 1123 | GAGGUCCAGGGUCUCAGGUCGUUUUAGAGCUAGAAAU | 1150 |
| | | | CAGGUC | | AGCAAGUUAAAAUAAGGCUAGUCCGUUAUCAACUUGA | |
| | | | | | AAAAGUGGCACCGAGUCGGUGCUUUU | |
| tap1_210 | TAP1 | CBE | GUAGAAUCCAGUCA | 1124 | GUAGAAUCCAGUCAGUGAGGGUUUUAGAGCUAGAAAU | 1151 |
| | | | GUGAGG | | AGCAAGUUAAAAUAAGGCUAGUCCGUUAUCAACUUGA | |
| | | | | | AAAAGUGGCACCGAGUCGGUGCUUUU | |
| tapbp_18 | TAPBP | CBE | GAACCAACACUCGA | 1125 | GAACCAACACUCGAUCACCGGUUUUAGAGCUAGAAAU | 1152 |
| | | | UCACCG | | AGCAAGUUAAAAUAAGGCUAGUCCGUUAUCAACUUGA | |
| | | | | | AAAAGUGGCACCGAGUCGGUGCUUUU | |
| tap2_4 | TAP2 | CBE | AGGUCCAGGGUCUC | 1126 | AGGUCCAGGGUCUCAGGUCAGUUUUAGAGCUAGAAAU | 1153 |
| | | | AGGUCA | | AGCAAGUUAAAAUAAGGCUAGUCCGUUAUCAACUUGA | |
| | | | | | AAAAGUGGCACCGAGUCGGUGCUUUU | |
| tapbp_5 | TAPBP | CBE | GCUCACCCAAAGCC | 1127 | GCUCACCCAAAGCCACAGCGGUUUUAGAGCUAGAAAU | 1154 |
| | | | ACAGCG | | AGCAAGUUAAAAUAAGGCUAGUCCGUUAUCAACUUGA | |
| | | | | | AAAAGUGGCACCGAGUCGGUGCUUUU | |
| tap 1_498 | TAP1 | CBE | CACCCAGCACCUCA | 1128 | CACCCAGCACCUCAGCCUGGGUUUUAGAGCUAGAAAU | 1155 |
| | | | GCCUGG | | AGCAAGUUAAAAUAAGGCUAGUCCGUUAUCAACUUGA | |
| | | | | | AAAAGUGGCACCGAGUCGGUGCUUUU | |
| tap2_181 | TAP2 | CBE | AAGCCAGUUACUCA | 1129 | AAGCCAGUUACUCAUCAGGGGUUUUAGAGCUAGAAAU | 1156 |
| | | | UCAGGG | | AGCAAGUUAAAAUAAGGCUAGUCCGUUAUCAACUUGA | |
| | | | | | AAAAGUGGCACCGAGUCGGUGCUUUU | |
| b2m 825 | B2M | CBE | ACUCACGCUGGAUA | 1130 | ACUCACGCUGGAUAGCCUCCGUUUUAGAGCUAGAAAU | 1157 |
| (TSBTx84 | | | GCCUCC | | AGCAAGUUAAAAUAAGGCUAGUCCGUUAUCAACUUGA | |
| 5) | | | | | AAAAGUGGCACCGAGUCGGUGCUUUU | |
| tap1_140 | TAP1 | CBE | UGUUCCGAGAGCUG | 1131 | UGUUCCGAGAGCUGAUCUCAGUUUUAGAGCUAGAAAU | 1158 |
| | | | AUCUCA | | AGCAAGUUAAAAUAAGGCUAGUCCGUUAUCAACUUGA | |
| | | | | | AAAAGUGGCACCGAGUCGGUGCUUUU | |
| tap1_202 | TAP2 | CBE | CCCCUGGAGAAAGA | 1132 | CCCCUGGAGAAAGAGAAGAGGUUUUAGAGCUAGAAAU | 1159 |
| | | | GAAGAG | | AGCAAGUUAAAAUAAGGCUAGUCCGUUAUCAACUUGA | |
| | | | | | AAAAGUGGCACCGAGUCGGUGCUUUU | |
| tapbp_64 | TAPBP | CBE | CCCUCCAGGCUGCC | 1133 | CCCUCCAGGCUGCCUUCAGGGUUUUAGAGCUAGAAAU | 1160 |
| | | | UUCAGG | | AGCAAGUUAAAAUAAGGCUAGUCCGUUAUCAACUUGA | |
| | | | | | AAAAGUGGCACCGAGUCGGUGCUUUU | |
| tap1_485 | TAP1 | CBE | CUCCACCUGAGGAA | 1134 | CUCCACCUGAGGAAAGACAUGUUUUAGAGCUAGAAAU | 1161 |
| | | | AGACAU | | AGCAAGUUAAAAUAAGGCUAGUCCGUUAUCAACUUGA | |
| | | | | | AAAAGUGGCACCGAGUCGGUGCUUUU | |
| tap1_142 | TAP1 | CBE | UUCCGAGAGCUGAU | 1135 | UUCCGAGAGCUGAUCUCAUGGUUUUAGAGCUAGAAAU | 1162 |
| | | | CUCAUG | | AGCAAGUUAAAAUAAGGCUAGUCCGUUAUCAACUUGA | |
| | | | | | AAAAGUGGCACCGAGUCGGUGCUUUU | |
| b2m 630 | B2M | CBE | AAGUCAACUUCAAU | 1136 | AAGUCAACUUCAAUGUCGGAGUUUUAGAGCUAGAAAU | 1163 |
| | | | GUCGGA | | AGCAAGUUAAAAUAAGGCUAGUCCGUUAUCAACUUGA | |
| | | | | | AAAAGUGGCACCGAGUCGGUGCUUUU | |
| tap2_325 | TAP2 | CBE | GCACCUGUCCCCCU | 1137 | GCACCUGUCCCCCUGUGGGCGUUUUAGAGCUAGAAAU | 1164 |
| | | | GUGGGC | | AGCAAGUUAAAAUAAGGCUAGUCCGUUAUCAACUUGA | |
| | | | | | AAAAGUGGCACCGAGUCGGUGCUUUU | |
| tap1_139 | TAP1 | CBE | CUCCCCAUGAGAUC | 1138 | CUCCCCAUGAGAUCAGCUCUGUUUUAGAGCUAGAAAU | 1165 |
| | | | AGCUCU | | AGCAAGUUAAAAUAAGGCUAGUCCGUUAUCAACUUGA | |
| | | | | | AAAAGUGGCACCGAGUCGGUGCUUUU | |
| tap1_38 | TAP1 | CBE | GGACUCUCCGCGCC | 1139 | GGACUCUCCGCGCCCCGGUGGUUUUAGAGCUAGAAAU | 1166 |
| | | | CCGGUG | | AGCAAGUUAAAAUAAGGCUAGUCCGUUAUCAACUUGA | |
| | | | | | AAAAGUGGCACCGAGUCGGUGCUUUU | |
| tapbp_79 | TAPBP | CBE | UGGCCCAUUUCGCA | 1140 | UGGCCCAUUUCGCAGAGGCGGUUUUAGAGCUAGAAAU | 1167 |
| | | | GAGGCG | | AGCAAGUUAAAAUAAGGCUAGUCCGUUAUCAACUUGA | |
| | | | | | AAAAGUGGCACCGAGUCGGUGCUUUU | |
| b2m 227 | B2M | CBE | GGAUUUGCCUAGCU | 1141 | GGAUUUGCCUAGCUUCCCCAGUUUUAGAGCUAGAAAU | 1168 |
| | | | UCCCCA | | AGCAAGUUAAAAUAAGGCUAGUCCGUUAUCAACUUGA | |
| | | | | | AAAAGUGGCACCGAGUCGGUGCUUUU | |
| tapbp_80 | TAPBP | CBE | CUGGCCCAUUUCGC | 1142 | CUGGCCCAUUUCGCAGAGGCGUUUUAGAGCUAGAAAU | 1169 |
| | | | AGAGGC | | AGCAAGUUAAAAUAAGGCUAGUCCGUUAUCAACUUGA | |
| | | | | | AAAAGUGGCACCGAGUCGGUGCUUUU | |
| b2m 596 | B2M | CBE | UCGAUCUAUGAAAA | 1143 | UCGAUCUAUGAAAAAGACAGGUUUUAGAGCUAGAAAU | 1170 |
| | | | AGACAG | | AGCAAGUUAAAAUAAGGCUAGUCCGUUAUCAACUUGA | |
| | | | | | AAAAGUGGCACCGAGUCGGUGCUUUU | |
| tap1_444 | TAP1 | CBE | GACCCAGAAGCCAA | 1144 | GACCCAGAAGCCAACUAUGGGUUUUAGAGCUAGAAAU | 1171 |
| | | | CUAUGG | | AGCAAGUUAAAAUAAGGCUAGUCCGUUAUCAACUUGA | |
| | | | | | AAAAGUGGCACCGAGUCGGUGCUUUU | |
| tap1_161 | TAP1 | CBE | UUGUGCCACAGGGC | 1145 | UUGUGCCACAGGGCUGCUGCGUUUUAGAGCUAGAAAU | 1172 |
| | | | UGCUGC | | AGCAAGUUAAAAUAAGGCUAGUCCGUUAUCAACUUGA | |
| | | | | | AAAAGUGGCACCGAGUCGGUGCUUUU | |
| tap1_454 | TAP1 | CBE | GUACCUGUGUCAUA | 1146 | GUACCUGUGUCAUAGCCCUGGUUUUAGAGCUAGAAAU | 1173 |
| | | | GCCCUG | | AGCAAGUUAAAAUAAGGCUAGUCCGUUAUCAACUUGA | |
| | | | | | AAAAGUGGCACCGAGUCGGUGCUUUU | |
| tap2_137 | TAP2 | CBE | CCUACCUGCCAAAG | 1147 | CCUACCUGCCAAAGGAGAAGGUUUUAGAGCUAGAAAU | 1174 |
| | | | GAGAAG | | AGCAAGUUAAAAUAAGGCUAGUCCGUUAUCAACUUGA | |
| | | | | | AAAAGUGGCACCGAGUCGGUGCUUUU | |

TABLE 1B
Guide RNAs

| | | Base | | | |
|---|---|---|---|---|---|
| Guide | | Editor/ | | | |
| Name | Gene | Nuclease | Spacer Sequence | PAM | gRNA sequence |
| CD58.1 | CD58 | ABE/CBE/ | GCAACCAUGGCUCGUCGGGC | CGG | GCAACCAUGGCUCGUCGGGCGUUUUAGAGCUAGAAAUAGCAAGUU |
| | | CABE/Cas9 | (SEQ ID NO: 672) | | AAAAUAAGGCUAGUCCGUUAUCAACUUGAAAAAGUGGCACCGA |
| | | | | | GUCGGUGCUUUU |
| | | | | | (SEQ ID NO: 675) |
| CD58.2 | CD58 | CBE/ABE/ | ACUCACCAAAGCAGUGCAGC | AGG | ACUCACCAAAGCAGUGCAGCGUUUUAGAGCUAGAAAUAGCAAGUU |
| | | CAS9 | (SEQ ID NO: 673) | | AAAAUAAGGCUAGUCCGUUAUCAACUUGAAAAAGUGGCACCGA |
| | | | | | GUCGGUGCUUUU |
| | | | | | (SEQ ID NO: 676) |
| CD58.3 | CD58 | CBE/ABE/ | CUCACCGCUGCUUGGGAUAC | AGG | CUCACCGCUGCUUGGGAUACGUUUUAGAGCUAGAAAUAGCAAGUU |
| | | Cas9 | (SEQ ID NO: 674) | | AAAAUAAGGCUAGUCCGUUAUCAACUUGAAAAAGUGGCACCGA |
| | | | | | GUCGGUGCUUUU |
| | | | | | (SEQ ID NO: 677) |

TABLE 1C
PAM sequences (in bold) corresponding to the guide RNA's of Table 1A

| Guide No. | PAM | gRNA Target + PAM | SEQ ID NO: |
|---|---|---|---|
| TSBTx4186 (846) | TGG | TGACGGCCATCCTCGGCGTCTGG | 1086 |
| TSBTx4187 (847) | GGG | GACGGCCATCCTCGGCGTCTGGG | 1087 |
| TSBTx4188 (848) | GGG | ACGGCCATCCTCGGCGTCTGGGG | 1088 |
| TSBTx4189 (849) | TGG | CGCACTCACCCGCCCAGGTCTGG | 1089 |
| TSBTx4190 (850) | GGG | GCACTCACCCGCCCAGGTCTGGG | 1090 |
| TSBTx4191 (851) | AGG | CCCAGGCTCGCACTCCATGAGG | 1091 |
| TSBTx4192 (852) | TGG | GTCACTCACCGGCCTCGCTCTGG | 1092 |
| TSBTx4193 (853) | AGG | CCTTACCCCATCTCAGGGTGAGG | 1093 |
| TSBTx4194 (854) | GGG | CTTACCCCATCTCAGGGTGAGGG | 1094 |
| TSBTx4195 (855) | AGG | GGCTCTGGGAAAAGAGGGGAAGG | 1095 |
| TSBTx4196 (856) | AGG | CTTCCCACAGATAGAAAAGGAGG | 1096 |
| TSBTx4197 (857) | GGG | TTCCCACAGATAGAAAAGGAGGG | 1097 |
| TSBTx4198 (858) | AGG | CCCAGGCAGTGACAGTGCCCAGG | 1098 |
| TSBTx4199 (859) | GGG | CCAGGCAGTGACAGTGCCCAGGG | 1099 |
| TSBTx4200 (860) | AGG | GACCCGCATCTCGGCGTCTGAGG | 1100 |
| TSBTx4201 (861) | CGG | GCACTCACCGGCCCAGGTCTCGG | 1101 |
| TSBTx4202 (862) | GGG | TGGGAGCCTGGGGGCGAGGAGGG | 1102 |
| TSBTx4203 (863) | GGG | GGGAGCCTGGGGGCGAGGAGGGG | 1103 |
| TSBTx4204 (864) | AGG | CCCCAGGCTCCCACTCCATGAGG | 1104 |
| TSBTx4205 (865) | GGG | TTACCCCATCTCAGGGTGAGGGG | 1105 |
| TSBTx4206 (866) | GGG | TTCCCACAGGTGGAAAAGGAGGG | 1106 |
| TSBTx4207 (867) | GGG | TGACCCGCATCTCGGCGTCTGGG | 1107 |
| TSBTx4208 (868) | CGG | GCACTCACAGGCCCAGGTCTCGG | 1108 |
| TSBTx4209 (869) | AGG | CACAGGCCCAGGTCTCGGTCAGG | 1109 |
| TSBTx4210 (870) | TGG | AGACCCTGGCCCCGCCCCCGTGG | 1110 |
| TSBTx4211 (871) | AGG | GGCTCTGGGAAAGGAGGGGAAGG | 1111 |
| TSBTx4212 (872) | AGG | CCCAGCCAGCAACAGTGCCCAGG | 1112 |
| TSBT×4167 (873) | ATTG | ATTGGGACCGGGAGACACAGAAGTAC | 1113 |
| TSBTx4168 (874) | ATTA | ATTACATCGCCCTGAACGAGGACCTG | 1114 |
| TSBTx4169 (875) | ATTA | ATTACATCGCCCTGAACGAGGATCTG | 1115 |
| TSBTx4170 (876) | ATTG | ATTGTTGCTGGCCTGGCTGTCCTAGC | 1116 |
| TSBTx4168 (877) | ATTA | ATTACATCGCCCTGAACGAGGACCTG | 1117 |

TABLE 1D
Exemplary guide RNAs

| Target | | | |
|---|---|---|---|
| Gene | gRNA sequence | SEQ ID NO | Editor |
| TRAC | GUUCUGUCUUUUGGUCAGGACAACCGUCUAGCUAUAAGUGC | 1175 | BhCas12b |
| | UGCAGGGUGUGAGAAACUCCUAUUGCUGGACGAUGUCUCUU | | nuclease |
| | ACGAGGCAUUAGCACAGAGUCUCUCAGCUGGUACAC | | |
| TRAC | GUUCUGUCUUUUGGUCAGGACAACCGUCUAGCUAUAAGUGC | 1176 | BhCas12b |
| | UGCAGGGUGUGAGGAAACUCCUAUUGCUGGACGAUGUCUCU | | nuclease |
| | UACGAGGCAUUAGCACACCGAUUUUGAUUCUCAAACA | | |
| TRAC | GUUCUGUCUUUUGGUCAGGACAACCGUCUAGCUAUAAGUGC | 1177 | BhCas12b |
| | UGCAGGGUGUGAGAAACUCCUAUUGCUGGACGAUGUCUCUU | | nuclease |
| | ACGAGGCAUUAGCACUCAAACAAAUGUGCACAAAG | | |
| TRAC | GUUCUGUCUUUUGGUCAGGACAACCGUCUAGCUAUAAGUGC | 1178 | BhCas12b |
| | UGCAGGGUGUGAGAAACUCCUAUUGCUGGACGAUGUCUCUU | | nuclease |
| | ACGAGGCAUUAGCACUCAAACAAAUGUGUCACAAAG | | |
| TRAC | GUUCUGUCUUUUGGUCAGGACAACCGUCUAGCUAUAAGUGC | 1179 | BhCas12b |
| | UGCAGGGUGUGAGAAACUCCUAUUGCUGGACGAUGUCUCUU | | nuclease |
| | ACGAGGCAUUAGCACUUUGAGAAUCAAAAUCGGUA | | |
| TRAC | GUUCUGUCUUUUGGUCAGGACAACCGUCUAGCUAUAAGUGC | 1180 | BhCas12b |
| | UGCAGGGUGUGAGAAACUCCUAUUGCUGGACGAUGUCUCUU | | nuclease |
| | ACGAGGCAUUAGCACUGAUGUGUAUAUCACAGACAA | | |
| TRAC | GUUCUGUCUUUUGGUCAGGACAACCGUCUAGCUAUAAGUGC | 1181 | BhCas12b |
| | UGCAGGGUGUGAGAAACUCCUAUUGCUGGACGAUGUCUCUU | | nuclease |
| | ACGAGGCAUUAGCAGUUGCUCCAGGCCACAGCAU | | |
| TRAC | GUUCUGUCUUUUGGUCAGGACAACCGUCUAGCUAUAAGUGC | 1182 | BhCas12b |
| | UGCAGGGUGUGAGAAACUCCUAUUGCUGGACGAUGUCUCUU | | nuclease |
| | ACGAGGCAUUAGCACUUCCAGAAGACACCUUCUUCC | | |
| TRAC | GUUCUGUCUUUUGGUCAGGACAACCGUCUAGCUAUAAGUGC | 1183 | BhCas12b |
| | UGCAGGGUGUGAGAAACUCCUAUUGCUGGACGAUGUCUCUU | | nuclease |
| | ACGAGGCAUUAGCACCAGAAGACACCUUCUUCCCCA | | |
| TRAC | GUUCUGUCUUUUGGUCAGGACAACCGUCUAGCUAUAAGUGC | 1184 | BhCas12b |
| | UGCAGGGUGUGAGAGAAACUCCUAUUGCUGGACGAUGUCUC | | nuclease |
| | UUACGAGGCAUUAGCACGGUUCCGAAUCCUCCUGA | | |
| TRAC | GUUCUGUCUUUUGGUCAGGACAACCGUCUAGCUAUAAGUGC | 1185 | BhCas12b |
| | UGCAGGGUGUGAGAAACUCCUAUUGCUGGACGAUGUCUCUU | | nuclease |
| | ACGAGGCAUUAGCACGGAACCCAAUCACUGACAGGU | | |
| TRAC | GUUCUGUCUUUUGGUCAGGACAACCGUCUAGCUAUAAGUGC | 1186 | BhCas12b |
| | UGCAGGGUGUGAGAAACUCCUAUUGCUGGACGAUGUCUCUU | | |
| | ACGAGGCAUUAGCACAGAGUCUCUCAGCUGGUACA | | |
| TRAC | GUUCUGUCUUUUGGUCAGGACAACCGUCUAGCUAUAAGUGC | 1187 | BhCas12b |
| | UGCAGGGUGUGAGAAACUCCUAUUGCUGGACGAUGUCUCUU | | |
| | ACGAGGCAUUAGCACACCGAUUUUGAUUCUCAAAC | | |
| TRAC | GUUCUGUCUUUUGGUCAGGACAACCGUCUAGCUAUAAGUGC | 1188 | BhCas12b |
| | UGCAGGGUGUGAGAAACUCCUAUUGCUGGACGAUGUCUCUU | | |
| | ACGAGGCAUUAGCACUGAUUCUCAAACAAAUGUGU | | |
| TRAC | GUUCUGUCUUUUGGUCAGGACAACCGUCUAGCUAUAAGUGC | 1189 | BhCas12b |
| | UGCAGGGUGUGAGAAACUCCUAUUGCUGGACGAUGUCUCUU | | |
| | ACGAGGCAUUAGCACUCAAACAAAUGUGUCACAAA | | |
| TRAC | GUUCUGUCUUUUGGUCAGGACAACCGUCUAGCUAUAAGUGC | 1190 | BhCas12b |
| | UGCAGGGUGUGAGAAACUCCUAUUGCUGGACGAUGUCUCUU | | |
| | ACGAGGCAUUAGCACGUUUGAGAAUCAAAAUCGGU | | |
| TRAC | GUUCUGUCUUUUGGUCAGGACAACCGUCUAGCUAUAAGUGC | 1191 | BhCas12b |
| | UGCAGGGUGUGAGAAACUCCUAUUGCUGGACGAUGUCUCUU | | |
| | ACGAGGCAUUAGCACUGAUGUGUAUAUCACAGACA | | |
| TRAC | GUUCUGUCUUUUGGUCAGGACAACCGUCUAGCUAUAAGUGC | 1192 | BhCas12b |
| | UGCAGGGUGUGAGAAACUCCUAUUGCUGGACGAUGUCUCUU | | |
| | ACGAGGCAUUAGCACGUUGCUCCAGGCCACAGCAC | | |
| TRAC | GUUCUGUCUUUUGGUCAGGACAACCGUCUAGCUAUAAGUGC | 1193 | BhCas12b |
| | UGCAGGGUGUGAGAAACUCCUAUUGCUGGACGAUGUCUCUU | | |
| | ACGAGGCAUUAGCACUUCCAGAAGACACCUUCUUC | | |
| TRAC | GUUCUGUCUUUUGGUCAGGACAACCGUCUAGCUAUAAGUGC | 1194 | BhCas12b |
| | UGCAGGGUGUGAGAAACUCCUAUUGCUGGACGAUGUCUCUU | | |
| | ACGAGGCAUUAGCACCAGAAGACACCUUCUUCCCC | | |
| TRAC | GUUCUGUCUUUUGGUCAGGACAACCGUCUAGCUAUAAGUGC | 1195 | BhCas12b |
| | UGCAGGGUGUGAGAAACUCCUAUUGCUGGACGAUGUCUCUU | | |
| | ACGAGGCAUUAGCACGGUUCCGAAUCCUCCUCCUG | | |
| TRAC | GUUCUGUCUUUUGGUCAGGACAACCGUCUAGCUAUAAGUGC | 1196 | BhCas12b |
| | UGCAGGGUGUGAGAAACUCCUAUUGCUGGACGAUGUCUCUU | | |
| | ACGAGGCAUUAGCACGGAACCCAAUCACUGACAGG | | |
| TRAC | GACCUAUAGGGUCAAUGAAUCUGUGCGUGUGCCAUAAGUAA | 1197 | BvCas12b |
| | UUAAAAAUUACCCACCACAGGAGCACCUGAAAACAGGUGCU | | |
| | UGGCACAGAGUCUCUCAGCUGGUACA | | |
| TRAC | GACCUAUAGGGUCAAUGAAUCUGUGCGUGUGCCAUAAGUAA | 1198 | BvCas12b |
| | UUAAAAAUUACCCACCACAGGAGCACCUGAAAACAGGUGCU | | |
| | UGGCACACCGAUUUUGAUUCUCAAAC | | |
| TRAC | GACCUAUAGGGUCAAUGAAUCUGUGCGUGUGCCAUAAGUAA | 1199 | BvCas12b |
| | UUAAAAAUUACCCACCACAGGAGCACCUGAAAACAGGUGCU | | |
| | UGGCACUGAUUCUCAAACAAAUGUGU | | |
| TRAC | GACCUAUAGGGUCAAUGAAUCUGUGCGUGUGCCAUAAGUAA | 1200 | BvCas12b |
| | UUAAAAAUUACCCACCACAGGAGCACCUGAAAACAGGUGCU | | |
| | UGGCACUCAAACAAAUGUGUCACAAA | | |
| TRAC | GACCUAUAGGGUCAAUGAAUCUGUGCGUGUGCCAUAAGUAA | 1201 | BvCas12b |
| | UUAAAAAUUACCCACCACAGGAGCACCUGAAAACAGGUGCU | | |
| | UGGCACGUUUGAGAAUCAAAAUCGGU | | |
| TRAC | GACCUAUAGGGUCAAUGAAUCUGUGCGUGUGCCAUAAGUAA | 1202 | BvCas12b |
| | UUAAAAAUUACCCACCACAGGAGCACCUGAAAACAGGUGCU | | |
| | UGGCACUGAUGUGUAUAUCACAGACA | | |
| TRAC | GACCUAUAGGGUCAAUGAAUCUGUGCGUGUGCCAUAAGUAA | 1203 | BvCas12b |
| | UUAAAAAUUACCCACCACAGGAGCACCUGAAAACAGGUGCU | | |
| | UGGCACGUUGCUCCAGGCCACAGCAC | | |
| TRAC | GACCUAUAGGGUCAAUGAAUCUGUGCGUGUGCCAUAAGUAA | 1204 | BvCas12b |
| | UUAAAAAUUACCCACCACAGGAGCACCUGAAAACAGGUGCU | | |
| | UGGCACUUCCAGAAGACACCUUCUUC | | |
| TRAC | GACCUAUAGGGUCAAUGAAUCUGUGCGUGUGCCAUAAGUAA | 1205 | BvCas12b |
| | UUAAAAAUUACCCACCACAGGAGCACCUGAAAACAGGUGCU | | |
| | UGGCACCAGAAGACACCUUCUUCCCC | | |
| TRAC | GACCUAUAGGGUCAAUGAAUCUGUGCGUGUGCCAUAAGUAA | 1206 | BvCas12b |
| | UUAAAAAUUACCCACCACAGGAGCACCUGAAAACAGGUGCU | | |
| | UGGCACGGUUCCGAAUCCUCCUCCUG | | |
| TRAC | GACCUAUAGGGUCAAUGAAUCUGUGCGUGUGCCAUAAGUAA | 1207 | BvCas12b |
| | UUAAAAAUUACCCACCACAGGAGCACCUGAAAACAGGUGCU | | |
| | UGGCACGGAACCCAAUCACUGACAGG | | |
| TRAC | UUCGUAUCUGUAAAACCAAGGUUUUAGAGCUAGAAAUAGCA | 1208 | CBE, spCas9 |
| | AGUUAAAAUAAGGCUAGUCCGUUAUCAACUUGAAAAAGUGG | | |
| | CACCGAGUCGGUGCUUUU | | |
| CD52 | UUACCUGUACCAUAACCAGGGUUUUAGAGCUAGAAAUAGCA | 1209 | CBE, spCas9 |
| | AGUUAAAAUAAGGCUAGUCCGUUAUCAACUUGAAAAAGUGG | | |
| | CACCGAGUCGGUGCUUUU | | |
| CD52 | CUCUUACCUGUACCAUAACCGUUUUAGAGCUAGAAAUAGCA | 1210 | CBE, spCas9 |
| | AGUUAAAAUAAGGCUAGUCCGUUAUCAACUUGAAAAAGUGG | | |
| | CACCGAGUCGGUGCUUUU | | |
| CD7 | UGCACCUCUGGGGAGGACCUGUUUUAGAGCUAGAAAUAGCA | 1211 | CBE, spCas9 |
| | AGUUAAAAUAAGGCUAGUCCGUUAUCAACUUGAAAAAGUGG | | |
| | CACCGAGUCGGUGCUUUU | | |
| CD7 | CCUACCUGUCACCAGGACCAGUUUUAGAGCUAGAAAUAGCA | 1212 | CBE, spCas9 |
| | AGUUAAAAUAAGGCUAGUCCGUUAUCAACUUGAAAAAGUGG | | |
| | CACCGAGUCGGUGCUUUU | | |
| PD1 | CACCUACCUAAGAACCAUCCGUUUUAGAGCUAGAAAUAGCA | 1213 | CBE, spCas9 |
| | AGUUAAAAUAAGGCUAGUCCGUUAUCAACUUGAAAAAGUGG | | |
| | CACCGAGUCGGUGCUUUU | | |

TABLE 1E
Exemplary Spacer Sequences

| | | Length | | SEQ | | |
|---|---|---|---|---|---|---|
| ID | Target | (bp) | Spacer Sequence | ID NO | PAM | Editor |
| sgRNA990 | HLA | 21 | UCCUUACCCCAUCUCAGGGUG | 447 | AGG | ABE/CBE/CABE |
| | class-I | | | | | |
| sgRNA991 | HLA | 22 | CUCCUUACCCCAUCUCAGGGUG | 448 | AGG | ABE/CBE/CABE |
| | class-I | | | | | |
| sgRNA992 | HLA | 19 | CUUACCCCAUCUCAGGGUG | 449 | AGG | ABE/CBE/CABE |
| | class-I | | | | | |
| sgRNA993 | HLA | 18 | UUACCCCAUCUCAGGGUG | 450 | AGG | ABE/CBE/CABE |
| | class-I | | | | | |
| sgRNA994 | HLA | 21 | CCUUACCCCAUCUCAGGGUGA | 451 | GGG | ABE/CBE/CABE |
| | class-I | | | | | |
| sgRNA995 | HLA | 22 | UCACCCUGAGAUGGGGUAAGGA | 452 | GGG | ABE/CBE/CABE |
| | class-I | | | | | |
| sgRNA996 | HLA | 19 | UUACCCCAUCUCAGGGUGA | 453 | GGG | ABE/CBE/CABE |
| | class-I | | | | | |
| sgRNA997 | HLA | 18 | UACCCCAUCUCAGGGUGA | 454 | GGG | ABE/CBE/CABE |
| | class-I | | | | | |

In some embodiments, provided herein is an immune cell with at least one modification in an endogenous gene or regulatory elements thereof. In some embodiments, the immune cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is implemented by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exons. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the immune cell is a T cell. In some embodiments, the immune cell is a CAR-T cell. In some embodiments, the immune cell is a NK cell.

In some embodiments, an edited gene may be an immune response regulation gene, an immunogenic gene, a checkpoint inhibitor gene, a gene involved in immune responses, a cell surface marker, e.g. a T cell surface marker, or any combination thereof. In some embodiments, the edited gene is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene.

In some embodiments, the editing of the endogenous gene reduces expression of the gene. In some embodiments, the editing of the endogenous gene reduces expression of the gene by at least 50% as compared to a control cell without the modification. In some embodiments, the editing of the endogenous gene reduces expression of the gene by at least 60% as compared to a control cell without the modification. In some embodiments, the editing of the endogenous gene reduces expression of the gene by at least 70% as compared to a control cell without the modification. In some embodiments, the editing of the endogenous gene reduces expression of the gene by at least 80% as compared to a control cell without the modification. In some embodiments, the editing of the endogenous gene reduces expression of the gene by at least 90% as compared to a control cell without the modification. In some embodiments, the editing of the endogenous gene reduces expression of the gene by at least 100% as compared to a control cell without the modification. In some embodiments, the editing of the endogenous gene eliminates gene expression.

In some embodiments, base editing may be performed on an intron. For example, base editing may be performed on an intron. In some embodiments, the base editing may be performed at a site within an intron. In some embodiments, the base editing may be performed at a site one or more introns. In some embodiments, the base editing may be performed at any exon of the multiple introns in a gene. In some embodiments, one or more base editing may be performed on an exon, an intron or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression. In some embodiments, base editing may generate a splice acceptor-splice donor (SA-SD) site. Targeted base editing generating a SA-SD, or at a SA-SD site can result in reduced expression of a gene. In some embodiments, base editors (e.g., ABE, CBE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., ABE, CBE) are used to edit exons by creating STOP codons.

In some embodiments, provided herein is an immune cell with at least one modification in one or more endogenous genes. In some embodiments, the immune cell may have at least one modification in one, two, three, four, five, six, seven, eight, nine, ten, eleven, twelve, thirteen, fourteen, fifteen, sixteen, seventeen, eighteen, nineteen, twenty or more endogenous genes. In some embodiments, the modification generates a premature stop codon in the endogenous genes. In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by a adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA; TGG→TAG; or TGG→TAA.

In some embodiments, the modification is a missense mutation. In some embodiments, the modification is in a peptide binding site, ATP binding site, splice site, promoter, enhancer, or in an untranslated region (UTR). In some embodiments, modification/base edits may be introduced at a 3′-UTR, for example, in a poly adenylation (poly-A) site. In some embodiments, base editing may be performed on a 5′-UTR region.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

In certain embodiments, the nucleobase editors provided herein comprise one or more features that improve base editing activity. For example, any of the nucleobase editors provided herein may comprise a Cas9 domain that has reduced nuclease activity. In some embodiments, any of the nucleobase editors provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand opposite the targeted nucleobase. Mutation of the catalytic residue (e.g., D10 to A10) prevents cleavage of the edited (e.g., deaminated) strand containing the targeted residue (e.g., A or C). Such Cas9 variants can generate a single-strand DNA break (nick) at a specific location based on the gRNA-defined target sequence, leading to repair of the non-edited strand, ultimately resulting in a nucleobase change on the non-edited strand.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease. An endonuclease can cleave a single strand of a double-stranded nucleic acid or both strands of a double-stranded nucleic acid molecule. In some embodiments, a nuclease domain of a polynucleotide programmable nucleotide binding domain can cut zero, one, or two strands of a target polynucleotide.

Non-limiting examples of a polynucleotide programmable nucleotide binding domain which can be incorporated into a base editor include a CRISPR protein-derived domain, a restriction nuclease, a meganuclease, TAL nuclease (TALEN), and a zinc finger nuclease (ZFN). In some embodiments, a base editor comprises a polynucleotide programmable nucleotide binding domain comprising a natural or modified protein or portion thereof which via a bound guide nucleic acid is capable of binding to a nucleic acid sequence during CRISPR (i.e., Clustered Regularly Interspaced Short Palindromic Repeats)-mediated modification of a nucleic acid. Such a protein is referred to herein as a “CRISPR protein.” Accordingly, disclosed herein is a base editor comprising a polynucleotide programmable nucleotide binding domain comprising all or a portion (e.g., a functional portion) of a CRISPR protein (i.e. a base editor comprising as a domain all or a portion (e.g., a functional portion) of a CRISPR protein, also referred to as a “CRISPR protein-derived domain” of the base editor). A CRISPR protein-derived domain incorporated into a base editor can be modified compared to a wild-type or natural version of the CRISPR protein. For example, as described below a CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2cl (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Cas9 nuclease sequences and structures are well known to those of skill in the art (See, e.g., “Complete genome sequence of an Ml strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### High Fidelity Cas9 Domains

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233. In some embodiments, high fidelity Cas9 domains are engineered Cas9 domains comprising one or more mutations that decrease electrostatic interactions between the Cas9 domain and the sugar-phosphate backbone of a DNA, relative to a corresponding wild-type Cas9 domain. High fidelity Cas9 domains that have decreased electrostatic interactions with the sugar-phosphate backbone of DNA have less off-target effects. In some embodiments, the Cas9 domain (e.g., a wild type Cas9 domain (SEQ ID NOs: 197 and 200)) comprises one or more mutations that decrease the association between the Cas9 domain and the sugar-phosphate backbone of a DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid. In some embodiments, the high fidelity Cas9 enzyme is SpCas9 (K855A), eSpCas9(1.1), SpCas9-HF1, or hyper accurate Cas9 variant (HypaCas9). In some embodiments, the modified Cas9 eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9's interactions with the DNA phosphate backbone. HypaCas9 contains mutations (SpCas9 N692A/M694A/Q695A/H698A) in the REC3 domain that increase Cas9 proofreading and target discrimination. All three high fidelity enzymes generate less off-target editing than wildtype Cas9.

Cas9 Domains with Reduced Exclusivity

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. This may limit the ability to edit desired bases within a genome. In some embodiments, the base editing fusion proteins or complexes provided herein may need to be placed at a precise location, for example a region comprising a target base that is upstream of the PAM. See e.g., Komor, A. C., et al., “Programmable editing of a target base in genomic DNA without double-stranded DNA cleavage” Nature 533, 420-424 (2016), the entire contents of which are hereby incorporated by reference. Exemplary polypeptide sequences for spCas9 proteins capable of binding a PAM sequence are provided in the Sequence Listing as SEQ ID NOs: 197, 201, and 234-237. Accordingly, in some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

### Nickases

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). In some embodiments, a nickase can be derived from a fully catalytically active (e.g., natural) form of a polynucleotide programmable nucleotide binding domain by introducing one or more mutations into the active polynucleotide programmable nucleotide binding domain. For example, where a polynucleotide programmable nucleotide binding domain comprises a nickase domain derived from Cas9, the Cas9-derived nickase domain can include a D10A mutation and a histidine at position 840. In such embodiments, the residue H840 retains catalytic activity and can thereby cleave a single strand of the nucleic acid duplex. In another example, a Cas9-derived nickase domain comprises an H840A mutation, while the amino acid residue at position 10 remains a D. In some embodiments, a nickase can be derived from a fully catalytically active (e.g., natural) form of a polynucleotide programmable nucleotide binding domain by removing all or a portion (e.g., a functional portion) of a nuclease domain that is not required for the nickase activity. For example, where a polynucleotide programmable nucleotide binding domain comprises a nickase domain derived from Cas9, the Cas9-derived nickase domain comprises a deletion of all or a portion (e.g., a functional portion) of the RuvC domain or the HNH domain.

In some embodiments, wild-type Cas9 corresponds to, or comprises the following amino acid sequence:

(SEQ ID NO: 197)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILOTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKROLVETROITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD
(single underline: HNH domain; double underline:
RuvC domain).

In some embodiments, the strand of a nucleic acid duplex target polynucleotide sequence that is cleaved by a base editor comprising a nickase domain (e.g., Cas9-derived nickase domain, Cas12-derived nickase domain) is the strand that is not edited by the base editor (i.e., the strand that is cleaved by the base editor is opposite to a strand comprising a base to be edited). In other embodiments, a base editor comprising a nickase domain (e.g., Cas9-derived nickase domain, Cas12-derived nickase domain) can cleave the strand of a DNA molecule which is being targeted for editing. In such embodiments, the non-targeted strand is not cleaved.

In some embodiments, a Cas9 nuclease has an inactive (e.g., an inactivated) DNA cleavage domain, that is, the Cas9 is a nickase, referred to as an “nCas9” protein (for “nickase” Cas9). The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840. In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10, or a corresponding mutation. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

The amino acid sequence of an exemplary catalytically Cas9 nickase (nCas9) is as follows:

(SEQ ID NO: 201)
MDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

The Cas9 nuclease has two functional endonuclease domains: RuvC and HNH. Cas9 undergoes a conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways: (1) the efficient but error-prone non-homologous end joining (NHEJ) pathway; or (2) the less efficient but high-fidelity homology directed repair (HDR) pathway.

The “efficiency” of non-homologous end joining (NHEJ) and/or homology directed repair (HDR) can be calculated by any convenient method. For example, in some embodiments, efficiency can be expressed in terms of percentage of successful HDR. For example, a surveyor nuclease assay can be used to generate cleavage products and the ratio of products to substrate can be used to calculate the percentage. For example, a surveyor nuclease enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., (b+c)/(a+b+c), where “a” is the band intensity of DNA substrate and “b” and “c” are the cleavage products).

In some embodiments, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1−(1−(b+c)/(a+b+c))^{1/2})×100, where “a” is the band intensity of DNA substrate and “b” and “c” are the cleavage products (Ran et. al., Cell. 2013 Sep. 12; 154(6):1380-9; and Ran et al., Nat Protoc. 2013 November; 8(11): 2281-2308).

The NHEJ repair pathway is the most active repair mechanism, and it frequently causes small nucleotide insertions or deletions (indels) at the DSB site. The randomness of NHEJ-mediated DSB repair has important practical implications, because a population of cells expressing Cas9 and a gRNA or a guide polynucleotide can result in a diverse array of mutations. In most embodiments, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

While NHEJ-mediated DSB repair often disrupts the open reading frame of the gene, homology directed repair (HDR) can be used to generate specific nucleotide changes ranging from a single nucleotide change to large insertions like the addition of a fluorophore or tag.

In order to utilize HDR for gene editing, a DNA repair template containing the desired sequence can be delivered into the cell type of interest with the gRNA(s) and Cas9 or Cas9 nickase. The repair template can contain the desired edit as well as additional homologous sequence immediately upstream and downstream of the target (termed left & right homology arms). The length of each homology arm can be dependent on the size of the change being introduced, with larger insertions requiring longer homology arms. The repair template can be a single-stranded oligonucleotide, double-stranded oligonucleotide, or a double-stranded DNA plasmid. The efficiency of HDR is generally low (<10% of modified alleles) even in cells that express Cas9, gRNA and an exogenous repair template. The efficiency of HDR can be enhanced by synchronizing the cells, since HDR takes place during the S and G2 phases of the cell cycle. Chemically or genetically inhibiting genes involved in NHEJ can also increase HDR frequency.

In some embodiments, Cas9 is a modified Cas9. A given gRNA targeting sequence can have additional sites throughout the genome where partial homology exists. These sites are called off-targets and need to be considered when designing a gRNA. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. The nickase system can also be combined with HDR-mediated gene editing for specific gene edits.

### Catalytically Dead Nucleases

Also provided herein are base editors comprising a polynucleotide programmable nucleotide binding domain which is catalytically dead (i.e., incapable of cleaving a target polynucleotide sequence). Herein the terms “catalytically dead” and “nuclease dead” are used interchangeably to refer to a polynucleotide programmable nucleotide binding domain which has one or more mutations and/or deletions resulting in its inability to cleave a strand of a nucleic acid. In some embodiments, a catalytically dead polynucleotide programmable nucleotide binding domain base editor can lack nuclease activity as a result of specific point mutations in one or more nuclease domains. For example, in the case of a base editor comprising a Cas9 domain, the Cas9 can comprise both a D10A mutation and an H840A mutation. Such mutations inactivate both nuclease domains, thereby resulting in the loss of nuclease activity. In other embodiments, a catalytically dead polynucleotide programmable nucleotide binding domain comprises one or more deletions of all or a portion (e.g., a functional portion) of a catalytic domain (e.g., RuvC1 and/or HNH domains). In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

In some embodiments, dCas9 corresponds to, or comprises in part or in whole, a Cas9 amino acid sequence having one or more mutations that inactivate the Cas9 nuclease activity. In some embodiments, the nuclease-inactive dCas9 domain comprises a D10X mutation and a H840X mutation of the amino acid sequence set forth herein, or a corresponding mutation in any of the amino acid sequences provided herein, wherein X is any amino acid change. In some embodiments, the nuclease-inactive dCas9 domain comprises a D10A mutation and a H840A mutation of the amino acid sequence set forth herein, or a corresponding mutation in any of the amino acid sequences provided herein. In some embodiments, a nuclease-inactive Cas9 domain comprises the amino acid sequence set forth in Cloning vector pPlatTET-gRNA2 (Accession No. BAV54124).

In some embodiments, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some embodiments, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

As another non-limiting example, in some embodiments, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 has restored catalytic His residue at position 840 in the Cas9 HNH domain (A840H).

As another non-limiting example, in some embodiments, the variant Cas9 protein harbors, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and W1126A mutations or when the variant Cas9 protein harbors P475A, W476A, N477A, D1125A, W1126A, and D1127A mutations, the variant Cas9 protein does not bind efficiently to a PAM sequence. Thus, in some such embodiments, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a variant Cas9 protein that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA.

In some embodiments, the variant Cas protein can be spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises a N579A mutation, or a corresponding mutation in any of the amino acid sequences provided in the Sequence Listing submitted herewith.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT or a NNGRRV PAM sequence. In some embodiments, the SaCas9 domain comprises one or more of a E781X, a N967X, and a R1014X mutation, or a corresponding mutation in any of the amino acid sequences provided herein, wherein X is any amino acid. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein.

In some embodiments, one of the Cas9 domains present in the fusion protein or complexes may be replaced with a guide nucleotide sequence-programmable DNA-binding protein domain that has no requirements for a PAM sequence. In some embodiments, the Cas9 is an SaCas9. Residue A579 of SaCas9 can be mutated from N579 to yield a SaCas9 nickase. Residues K781, K967, and H1014 can be mutated from E781, N967, and R1014 to yield a SaKKH Cas9.

In some embodiments, a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′ was used.

Alternatives to S. pyogenes Cas9 can include RNA-guided endonucleases from the Cpf1 family that display cleavage activity in mammalian cells. CRISPR from Prevotella and Francisella 1 (CRISPR/Cpf1) is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. Unlike Cas9 nucleases, the result of Cpf1-mediated DNA cleavage is a double-strand break with a short 3′ overhang. Cpf1's staggered cleavage pattern can open up the possibility of directional gene transfer, analogous to traditional restriction enzyme cloning, which can increase the efficiency of gene editing. Like the Cas9 variants and orthologues described above, Cpf1 can also expand the number of sites that can be targeted by CRISPR to AT-rich regions or AT-rich genomes that lack the NGG PAM sites favored by SpCas9. The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9.

Furthermore, Cpf1, unlike Cas9, does not have a HNH endonuclease domain, and the N-terminal of Cpf1 does not have the alpha-helical recognition lobe of Cas9. Cpf1 CRISPR-Cas domain architecture shows that Cpf1 is functionally unique, being classified as Class 2, type V CRISPR system. The Cpf1 loci encode Cas1, Cas2 and Cas4 proteins that are more similar to types I and III than type II systems. Functional Cpf1 does not require the trans-activating CRISPR RNA (tracrRNA), therefore, only CRISPR (crRNA) is required. This benefits genome editing because Cpf1 is not only smaller than Cas9, but also it has a smaller sgRNA molecule (approximately half as many nucleotides as Cas9). The Cpf1-crRNA complex cleaves target DNA or RNA by identification of a protospacer adjacent motif 5′-YTN-3′ or 5′-TTN-3′ in contrast to the G-rich PAM targeted by Cas9. After identification of PAM, Cpf1 introduces a sticky-end-like DNA double-stranded break having an overhang of 4 or 5 nucleotides.

In some embodiments, the Cas9 is a Cas9 variant having specificity for an altered PAM sequence. In some embodiments, the Additional Cas9 variants and PAM sequences are described in Miller, S. M., et al. Continuous evolution of SpCas9 variants compatible with non-G PAMs, Nat. Biotechnol. (2020), the entirety of which is incorporated herein by reference. in some embodiments, a Cas9 variate have no specific PAM requirements. In some embodiments, a Cas9 variant, e.g. a SpCas9 variant has specificity for a NRNH PAM, wherein R is A or G and H is A, C, or T. In some embodiments, the SpCas9 variant has specificity for a PAM sequence AAA, TAA, CAA, GAA, TAT, GAT, or CAC. In some embodiments, the SpCas9 variant comprises an amino acid substitution at position 1114, 1134, 1135, 1137, 1139, 1151, 1180, 1188, 1211, 1218, 1219, 1221, 1249, 1256, 1264, 1290, 1318, 1317, 1320, 1321, 1323, 1332, 1333, 1335, 1337, or 1339 or a corresponding position thereof. In some embodiments, the SpCas9 variant comprises an amino acid substitution at position 1114, 1135, 1218, 1219, 1221, 1249, 1320, 1321, 1323, 1332, 1333, 1335, or 1337 or a corresponding position thereof. In some embodiments, the SpCas9 variant comprises an amino acid substitution at position 1114, 1134, 1135, 1137, 1139, 1151, 1180, 1188, 1211, 1219, 1221, 1256, 1264, 1290, 1318, 1317, 1320, 1323, 1333 or a corresponding position thereof. In some embodiments, the SpCas9 variant comprises an amino acid substitution at position 1114, 1131, 1135, 1150, 1156, 1180, 1191, 1218, 1219, 1221, 1227, 1249, 1253, 1286, 1293, 1320, 1321, 1332, 1335, 1339 or a corresponding position thereof. In some embodiments, the SpCas9 variant comprises an amino acid substitution at position 1114, 1127, 1135, 1180, 1207, 1219, 1234, 1286, 1301, 1332, 1335, 1337, 1338, 1349 or a corresponding position thereof. Exemplary amino acid substitutions and PAM specificity of SpCas9 variants are shown in Tables 2A-2D).

TABLE 2A
SpCas9 Variants and PAM specificity

| | SpCas9 amino acid position | | | | | | | | | | | | |
|---|---|---|---|---|---|---|---|---|---|---|---|---|---|
| | 1114 | 1135 | 1218 | 1219 | 1221 | 1249 | 1320 | 1321 | 1323 | 1332 | 1333 | 1335 | 1337 |
| PAM | R | D | G | E | Q | P | A | P | A | D | R | R | T |
| AAA | | N | | V | H | | | | | | G | | |
| AAA | | N | | V | H | | | | | | G | | |
| AAA | | | | V | | | | | | | G | | |
| TAA | G | N | | V | | | | | | | I | | |
| TAA | | N | | V | | | | | | | I | | A |
| TAA | G | N | | V | | | | | | | I | | A |
| CAA | | | | V | | | | | | | K | | |
| CAA | | N | | V | | | | | | | K | | |
| CAA | | N | | V | | | | | | | K | | |
| GAA | | | | V | H | | V | | | | K | | |
| GAA | | N | | V | | | V | | | | K | | |
| GAA | | | | V | H | | V | | | | K | | |
| TAT | | | S | V | H | S | | S | | | | L | |
| TAT | | | S | V | H | S | | S | | | | L | |
| TAT | | | S | V | H | S | | S | | | | L | |
| GAT | | | | V | | | | | | | I | | |
| GAT | | | | V | | | | | D | | | Q | |
| GAT | | | | V | | | | | D | | | Q | |
| CAC | | | | V | | | | | | N | | Q | N |
| CAC | | N | | V | | | | | | | | Q | N |
| CAC | | | | V | | | | | | N | | Q | N |

TABLE 2B
SpCas9 Variants and PAM specificity

| | SpCas9 amino acid position | | | | | | | | | | | | | | | | | | |
|---|---|---|---|---|---|---|---|---|---|---|---|---|---|---|---|---|---|---|---|
| | 1114 | 1134 | 1135 | 1137 | 1139 | 1151 | 1180 | 1188 | 1211 | 1219 | 1221 | 1256 | 1264 | 1290 | 1318 | 1317 | 1320 | 1323 | 1333 |
| PAM | R | F | D | P | V | K | D | K | K | E | Q | Q | H | V | L | N | A | A | R |
| GAA | | | | | | | | | | V | H | | | | | | V | | K |
| GAA | | | N | S | | | | | | V | | | | | | | V | D | K |
| GAA | | | N | | | | | | | V | H | | Y | | | | V | | K |
| CAA | | | N | | | | | | | V | H | | Y | | | | V | | K |
| CAA | G | | N | S | | | | | | V | H | | Y | | | | V | | K |
| CAA | | | N | | | | | R | | V | H | | | | | | V | | K |
| CAA | | | N | | | | G | | R | V | H | | Y | | | | V | | K |
| CAA | | | N | | | | | | | V | H | | Y | | | | V | | K |
| AAA | | | N | | | | G | | | V | H | R | Y | | | | V | D | K |
| CAA | G | | N | | | | G | | | V | H | | Y | | | | V | D | K |
| CAA | | L | N | | | | G | | | V | H | | Y | | | T | V | D | K |
| TAA | G | | N | | | | G | | | V | H | | Y | G | S | | V | D | K |
| TAA | G | | N | | | E | G | | | V | H | | Y | | S | | V | | K |
| TAA | G | | N | | | | G | | | V | H | | Y | | S | | V | D | K |
| TAA | G | | N | | | | G | | R | V | H | | | | | | V | | K |
| TAA | | | N | | | | G | | R | V | H | | Y | | | | V | | K |
| TAA | G | | N | | A | | G | | | V | H | | | | | | V | | K |
| TAA | G | | N | | | | | | | V | H | | | | | | V | | K |

TABLE 2C
SpCas9 Variants and PAM Specificity

| | SpCas9 amino acid position | | | | | | | | | |
|---|---|---|---|---|---|---|---|---|---|---|
| | 1114 | 1131 | 1135 | 1150 | 1156 | 1180 | 1191 | 1218 | 1219 | 1221 |
| PAM | R | Y | D | E | K | D | K | G | E | Q |
| SacB.TAT | | | N | | | | N | | V | H |
| SacB.TAT | | | N | | | | | S | V | H |
| AAT | | | N | | | | | S | V | H |
| TAT | G | | N | | | G | | S | V | H |
| TAT | G | | N | | | G | | S | V | H |
| TAT | G | C | N | | | G | | S | V | H |
| TAT | G | C | N | | | G | | S | V | H |
| TAT | G | C | N | | | G | | S | V | H |
| TAT | G | C | N | | E | G | | S | V | H |
| TAT | G | C | N | V | | G | | S | V | H |
| TAT | | C | N | | | G | | S | V | H |
| TAT | G | C | N | | | G | | S | V | H |

| | SpCas9 amino acid position | | | | | | | | | |
|---|---|---|---|---|---|---|---|---|---|---|
| | 1227 | 1249 | 1253 | 1286 | 1293 | 1320 | 1321 | 1332 | 1335 | 1339 |
| PAM | A | P | E | N | A | A | P | D | R | T |
| SacB.TAT | | | | | | V | S | | L | |
| SacB.TAT | | S | | | | | S | G | L | |
| AAT | V | S | | K | T | | S | G | L | I |
| TAT | | S | K | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |

TABLE 2D
SpCas9 Variants and PAM Specificity acid

| | SpCas9 amino acid position | | | | | | | | | | | | | |
|---|---|---|---|---|---|---|---|---|---|---|---|---|---|---|
| | 1114 | 1127 | 1135 | 1180 | 1207 | 1219 | 1234 | 1286 | 1301 | 1332 | 1335 | 1337 | 1338 | 1349 |
| PAM | R | D | D | D | E | E | N | N | P | D | R | T | S | H |

| | | | | | | | | | | | | | |
|---|---|---|---|---|---|---|---|---|---|---|---|---|---|
| SacB.CAC | | | N | | | V | | | N | Q | N | | |
| AAC | G | | N | | | V | | | N | Q | N | | |
| AAC | G | | N | | | V | | | N | Q | N | | |
| TAC | G | | N | | | V | | | N | Q | N | | |
| TAC | G | | N | | | V | | H | N | Q | N | | |
| TAC | G | | N | | G | V | D | H | N | Q | N | | |
| TAC | G | | N | | | V | | | N | Q | N | | |
| TAC | G | G | N | E | | V | | H | N | Q | N | | |
| TAC | G | | N | | | V | | H | N | Q | N | | |
| TAC | G | | N | | | V | | | N | Q | N | T | R |

Further exemplary Cas9 (e.g., SaCas9) polypeptides with modified PAM recognition are described in Kleinstiver, et al. “Broadening the targeting range of Staphylococcus aureus CRISPR-Cas9 by modifying PAM recognition,” Nature Biotechnology, 33:1293-1298 (2015) DOI: 10.1038/nbt.3404, the disclosure of which is incorporated herein by reference in its entirety for all purposes. In some embodiments, a Cas9 variant (e.g., a SaCas9 variant) comprising one or more of the alterations E782K, N929R, N968K, and/or R1015H has specificity for, or is associated with increased editing activities relative to a reference polypeptide (e.g., SaCas9) at an NNNRRT or NNHRRT PAM sequence, where N represents any nucleotide, H represents any nucleotide other than G (i.e., “not G”), and R represents a purine. In embodiments, the Cas9 variant (e.g., a SaCas9 variant) comprises the alterations E782K, N968K, and R1015H or the alterations E782K, K929R, and R1015H.

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, Cas12b/C2c1, and Cas12c/C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (Cas12b/C2c1, and Cas12c/C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, Cas12b/C2c1, and Cas12c/C2c3, contain RuvC-like endonuclease domains related to Cpf1. A third system contains an effector with two predicated HEPN RNase domains. Production of mature CRISPR RNA is tracrRNA-independent, unlike production of CRISPR RNA by Cas12b/C2cl. Cas12b/C2cl depends on both CRISPR RNA and tracrRNA for DNA cleavage.

In some embodiments, the napDNAbp is a circular permutant (e.g., SEQ ID NO: 238).

The crystal structure of Alicyclobaccillus acidoterrastris Cas12b/C2cl (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2cl bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with Cas12b/C2c1-mediated cleavage resulting in a staggered seven-nucleotide break of target DNA. Structural comparisons between Cas12b/C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins or complexes provided herein may be a Cas12b/C2c1, or a Cas12c/C2c3 protein. In some embodiments, the napDNAbp is a Cas12b/C2cl protein. In some embodiments, the napDNAbp is a Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 or Cas12c/C2c3 protein. In some embodiments, the napDNAbp is a naturally-occurring Cas12b/C2c1 or Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the napDNAbp sequences provided herein. It should be appreciated that Cas12b/C2c1 or Cas12c/C2c3 from other bacterial species may also be used in accordance with the present disclosure.

In some embodiments, a napDNAbp refers to Cas12c. In some embodiments, the Cas12c protein is a Cas12cl (SEQ ID NO: 239) or a variant of Cas12c1. In some embodiments, the Cas12 protein is a Cas12c2 (SEQ ID NO: 240) or a variant of Cas12c2. In some embodiments, the Cas12 protein is a Cas12c protein from Oleiphilus sp. H10009 (i.e., OspCas12c; SEQ ID NO: 241) or a variant of OspCas12c. These Cas12c molecules have been described in Yan et al., “Functionally Diverse Type V CRISPR-Cas Systems,” Science, 2019 Jan. 4; 363: 88-91; the entire contents of which is hereby incorporated by reference. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12c1, Cas12c2, or OspCas12c protein. In some embodiments, the napDNAbp is a naturally-occurring Cas12c1, Cas12c2, or OspCas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12cl, Cas12c2, or OspCas12c protein described herein. It should be appreciated that Cas12c1, Cas12c2, or OspCas12c from other bacterial species may also be used in accordance with the present disclosure.

In some embodiments, a napDNAbp refers to Cas12g, Cas12h, or Cas12i, which have been described in, for example, Yan et al., “Functionally Diverse Type V CRISPR-Cas Systems,” Science, 2019 Jan. 4; 363: 88-91; the entire contents of each is hereby incorporated by reference. Exemplary Cas12g, Cas12h, and Cas12i polypeptide sequences are provided in the Sequence Listing as SEQ ID NOs: 242-245. By aggregating more than 10 terabytes of sequence data, new classifications of Type V Cas proteins were identified that showed weak similarity to previously characterized Class V protein, including Cas12g, Cas12h, and Cas12i. In some embodiments, the Cas12 protein is a Cas12g or a variant of Cas12g. In some embodiments, the Cas12 protein is a Cas12h or a variant of Cas12h. In some embodiments, the Cas12 protein is a Cas12i or a variant of Cas12i. It should be appreciated that other RNA-guided DNA binding proteins may be used as a napDNAbp, and are within the scope of this disclosure. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12g, Cas12h, or Cas12i protein. In some embodiments, the napDNAbp is a naturally-occurring Cas12g, Cas12h, or Cas12i protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12g, Cas12h, or Cas12i protein described herein. It should be appreciated that Cas12g, Cas12h, or Cas12i from other bacterial species may also be used in accordance with the present disclosure. In some embodiments, the Cas12i is a Cas12i1 or a Cas12i2.

In some embodiments, the nucleic acid programmable DNA binding protein (napDNAbp) of any of the fusion proteins or complexes provided herein may be a Cas12j/CasΦ protein. Cas12j/CasΦ is described in Pausch et al., “CRISPR-CasΦ from huge phages is a hypercompact genome editor,” Science, 17 Jul. 2020, Vol. 369, Issue 6501, pp. 333-337, which is incorporated herein by reference in its entirety. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12j/CasΦ protein. In some embodiments, the napDNAbp is a naturally-occurring Cas12j/CasΦ protein. In some embodiments, the napDNAbp is a nuclease inactive (“dead”) Cas12j/CasΦ protein. It should be appreciated that Cas12j/CasΦ from other species may also be used in accordance with the present disclosure.

Fusion Proteins or Complexes with Internal Insertions

Provided herein are fusion proteins or complexes comprising a heterologous polypeptide fused to a nucleic acid programmable nucleic acid binding protein, for example, a napDNAbp. A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or a Cas12 (e.g., Cas12b/C2cl), polypeptide. In some embodiments, the cytidine deaminase is an APOBEC deaminase (e.g., APOBEC1). In some embodiments, the adenosine deaminase is a TadA (e.g., TadA*7.10 or TadA*8). In some embodiments, the TadA is a TadA*8 or a TadA*9. TadA sequences (e.g., TadA7.10 or TadA*8) as described herein are suitable deaminases for the above-described fusion proteins or complexes.

In some embodiments, the fusion protein comprises the structure:
NH2-[N-terminal fragment of a napDNAbp]-[deaminase]-[C-terminal fragment of a napDNAbp]-COOH;
NH2-[N-terminal fragment of a Cas9]-[adenosine deaminase]-[C-terminal fragment of a Cas9]-COOH;
NH2-[N-terminal fragment of a Cas12]-[adenosine deaminase]-[C-terminal fragment of a Cas12]-COOH;
NH2-[N-terminal fragment of a Cas9]-[cytidine deaminase]-[C-terminal fragment of a Cas9]-COOH;
NH2-[N-terminal fragment of a Cas12]-[cytidine deaminase]-[C-terminal fragment of a Cas12]-COOH;
wherein each instance of “]-[” indicates the optional presence of a linker (i.e., the linker is optionally present).

The deaminase can be a circular permutant deaminase. For example, the deaminase can be a circular permutant adenosine deaminase. In some embodiments, the deaminase is a circular permutant TadA, circularly permutated at amino acid residue 116, 136, or 65 as numbered in the TadA reference sequence.

The fusion protein or complexes can comprise more than one deaminase. The fusion protein or complex can comprise, for example, 1, 2, 3, 4, 5 or more deaminases. In some embodiments, the fusion protein or complex comprises one or two deaminase. The two or more deaminases in a fusion protein or complex can be an adenosine deaminase, a cytidine deaminase, or a combination thereof. The two or more deaminases can be homodimers or heterodimers. The two or more deaminases can be inserted in tandem in the napDNAbp. In some embodiments, the two or more deaminases may not be in tandem in the napDNAbp.

In some embodiments, the napDNAbp in the fusion protein or complex is a Cas9 polypeptide or a fragment thereof. The Cas9 polypeptide can be a variant Cas9 polypeptide. In some embodiments, the Cas9 polypeptide is a Cas9 nickase (nCas9) polypeptide or a fragment thereof. In some embodiments, the Cas9 polypeptide is a nuclease dead Cas9 (dCas9) polypeptide or a fragment thereof. The Cas9 polypeptide in a fusion protein or complex can be a full-length Cas9 polypeptide. In some cases, the Cas9 polypeptide in a fusion protein or complex may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment, a portion, or a domain of a Cas9 polypeptide, that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus 1 Cas9 (St1Cas9), or fragments or variants of any of the Cas9 polypeptides described herein.

In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:
NH2-[Cas9(adenosine deaminase)]-[cytidine deaminase]-COOH;
NH2-[cytidine deaminase]-[Cas9(adenosine deaminase)]-COOH;
NH2-[Cas9(cytidine deaminase)]-[adenosine deaminase]-COOH; or
NH2-[adenosine deaminase]-[Cas9(cytidine deaminase)]-COOH.

In some embodiments, the “-” used in the general architecture above indicates the optional presence of a linker (i.e., the linker is optionally present).

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas9 and a cytidine deaminase is fused to the C-terminus. In some embodiments, a TadA*8 is fused within Cas9 and a cytidine deaminase fused to the N-terminus. In some embodiments, a cytidine deaminase is fused within Cas9 and a TadA*8 is fused to the C-terminus. In some embodiments, a cytidine deaminase is fused within Cas9 and a TadA*8 fused to the N-terminus. Exemplary structures of a fusion protein with a TadA*8 and a cytidine deaminase and a Cas9 are provided as follows:
NH2-[Cas9(TadA*8)]-[cytidine deaminase]-COOH;
NH2-[cytidine deaminase]-[Cas9(TadA*8)]—COOH;
NH2-[Cas9(cytidine deaminase)]-[TadA*8]—COOH; or
NH2-[TadA*8]-[Cas9(cytidine deaminase)]-COOH.

In some embodiments, the “-” used in the general architecture above indicates the optional presence of a linker (i.e., the linker is optionally present).

The heterologous polypeptide (e.g., deaminase) can be inserted in the napDNAbp (e.g., Cas9 or Cas12 (e.g., Cas12b/C2cl)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid). A deaminase (e.g., adenosine deaminase, cytidine deaminase, or adenosine deaminase and cytidine deaminase) can be inserted in the napDNAbp at, for example, a disordered region or a region comprising a high temperature factor or B-factor as shown by crystallographic studies. Regions of a protein that are less ordered, disordered, or unstructured, for example solvent exposed regions and loops, can be used for insertion without compromising structure or function. A deaminase (e.g., adenosine deaminase, cytidine deaminase, or adenosine deaminase and cytidine deaminase) can be inserted in the napDNAbp in a flexible loop region or a solvent-exposed region. In some embodiments, the deaminase (e.g., adenosine deaminase, cytidine deaminase, or adenosine deaminase and cytidine deaminase) is inserted in a flexible loop of the Cas9 or the Cas12b/C2cl polypeptide.

In some embodiments, the insertion location of a deaminase (e.g., adenosine deaminase, cytidine deaminase, or adenosine deaminase and cytidine deaminase) is determined by B-factor analysis of the crystal structure of Cas9 polypeptide. In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). B-factor or temperature factor can indicate the fluctuation of atoms from their average position (for example, as a result of temperature-dependent atomic vibrations or static disorder in a crystal lattice). A high B-factor (e.g., higher than average B-factor) for backbone atoms can be indicative of a region with relatively high local mobility. Such a region can be used for inserting a deaminase without compromising structure or function. A deaminase (e.g., adenosine deaminase, cytidine deaminase, or adenosine deaminase and cytidine deaminase) can be inserted at a location with a residue having a Cu atom with a B-factor that is 50%, 60%, 70%, 80%, 90%, 100%, 110%, 120%, 130%, 140%, 150%, 160%, 170%, 180%, 190%, 200%, or greater than 200% more than the average B-factor for the total protein. A deaminase (e.g., adenosine deaminase, cytidine deaminase, or adenosine deaminase and cytidine deaminase) can be inserted at a location with a residue having a Cu atom with a B-factor that is 50%, 60%, 70%, 80%, 90%, 100%, 110%, 120%, 130%, 140%, 150%, 160%, 170%, 180%, 190%, 200% or greater than 200% more than the average B-factor for a Cas9 protein domain comprising the residue. Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

A heterologous polypeptide (e.g., deaminase) can be inserted in the napDNAbp at an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. In some embodiments, the heterologous polypeptide is inserted between amino acid positions 768-769, 791-792, 792-793, 1015-1016, 1022-1023, 1026-1027, 1029-1030, 1040-1041, 1052-1053, 1054-1055, 1067-1068, 1068-1069, 1247-1248, or 1248-1249 as numbered in the above Cas9 reference sequence or corresponding amino acid positions thereof. In some embodiments, the heterologous polypeptide is inserted between amino acid positions 769-770, 792-793, 793-794, 1016-1017, 1023-1024, 1027-1028, 1030-1031, 1041-1042, 1053-1054, 1055-1056, 1068-1069, 1069-1070, 1248-1249, or 1249-1250 as numbered in the above Cas9 reference sequence or corresponding amino acid positions thereof. In some embodiments, the heterologous polypeptide replaces an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. It should be understood that the reference to the above Cas9 reference sequence with respect to insertion positions is for illustrative purposes. The insertions as discussed herein are not limited to the Cas9 polypeptide sequence of the above Cas9 reference sequence, but include insertion at corresponding locations in variant Cas9 polypeptides, for example a Cas9 nickase (nCas9), nuclease dead Cas9 (dCas9), a Cas9 variant lacking a nuclease domain, a truncated Cas9, or a Cas9 domain lacking partial or complete HNH domain.

A heterologous polypeptide (e.g., deaminase) can be inserted in the napDNAbp at an amino acid residue selected from the group consisting of: 768, 792, 1022, 1026, 1040, 1068, and 1247 as numbered in the above Cas9 reference sequence, or a corresponding amino acid residue in another Cas9 polypeptide. In some embodiments, the heterologous polypeptide is inserted between amino acid positions 768-769, 792-793, 1022-1023, 1026-1027, 1029-1030, 1040-1041, 1068-1069, or 1247-1248 as numbered in the above Cas9 reference sequence or corresponding amino acid positions thereof. In some embodiments, the heterologous polypeptide is inserted between amino acid positions 769-770, 793-794, 1023-1024, 1027-1028, 1030-1031, 1041-1042, 1069-1070, or 1248-1249 as numbered in the above Cas9 reference sequence or corresponding amino acid positions thereof. In some embodiments, the heterologous polypeptide replaces an amino acid residue selected from the group consisting of: 768, 792, 1022, 1026, 1040, 1068, and 1247 as numbered in the above Cas9 reference sequence, or a corresponding amino acid residue in another Cas9 polypeptide.

A heterologous polypeptide (e.g., deaminase) can be inserted in the napDNAbp at an amino acid residue as described herein, or a corresponding amino acid residue in another Cas9 polypeptide. In an embodiment, a heterologous polypeptide (e.g., deaminase) can be inserted in the napDNAbp at an amino acid residue selected from the group consisting of: 1002, 1003, 1025, 1052-1056, 1242-1247, 1061-1077, 943-947, 686-691, 569-578, 530-539, and 1060-1077 as numbered in the above Cas9 reference sequence, or a corresponding amino acid residue in another Cas9 polypeptide. The deaminase (e.g., adenosine deaminase, cytidine deaminase, or adenosine deaminase and cytidine deaminase) can be inserted at the N-terminus or the C-terminus of the residue or replace the residue. In some embodiments, the deaminase (e.g., adenosine deaminase, cytidine deaminase, or adenosine deaminase and cytidine deaminase) is inserted at the C-terminus of the residue.

In some embodiments, an adenosine deaminase (e.g., TadA) is inserted at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, an adenosine deaminase (e.g., TadA) is inserted in place of residues 792-872, 792-906, or 2-791 as numbered in the above Cas9 reference sequence, or a corresponding amino acid residue in another Cas9 polypeptide. In some embodiments, the adenosine deaminase is inserted at the N-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted at the C-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted to replace an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

In some embodiments, a cytidine deaminase (e.g., APOBEC1) is inserted at an amino acid residue selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the N-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the C-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted to replace an amino acid selected from the group consisting of: 1016, 1023, 1029, 1040, 1069, and 1247 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 768 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 791 or is inserted at amino acid residue 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid 791, or is inserted to replace amino acid 792, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1016 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1022, or is inserted at amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1022 or is inserted at the N-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1022 or is inserted at the C-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1022, or is inserted to replace amino acid residue 1023, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1026, or is inserted at amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1026 or is inserted at the N-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1026 or is inserted at the C-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1026, or is inserted to replace amino acid residue 1029, as numbered in the above Cas9 reference sequence, or corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1040 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1052, or is inserted at amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1052 or is inserted at the N-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1052 or is inserted at the C-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1052, or is inserted to replace amino acid residue 1054, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1067, or is inserted at amino acid residue 1068, or is inserted at amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1067 or is inserted at the N-terminus of amino acid residue 1068 or is inserted at the N-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1067 or is inserted at the C-terminus of amino acid residue 1068 or is inserted at the C-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1067, or is inserted to replace amino acid residue 1068, or is inserted to replace amino acid residue 1069, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1246, or is inserted at amino acid residue 1247, or is inserted at amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1246 or is inserted at the N-terminus of amino acid residue 1247 or is inserted at the N-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1246 or is inserted at the C-terminus of amino acid residue 1247 or is inserted at the C-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1246, or is inserted to replace amino acid residue 1247, or is inserted to replace amino acid residue 1248, as numbered in the above Cas9 reference sequence, or a corresponding amino acid residue in another Cas9 polypeptide.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. In some embodiments, the deleted region corresponds to residues 792-872 as numbered in the above Cas9 reference sequence, or a corresponding amino acid residue in another Cas9 polypeptide. In some embodiments, the deleted region corresponds to residues 792-906 as numbered in the above Cas9 reference sequence, or a corresponding amino acid residue in another Cas9 polypeptide. In some embodiments, the deleted region corresponds to residues 2-791 as numbered in the above Cas9 reference sequence, or a corresponding amino acid residue in another Cas9 polypeptide. In some embodiments, the deleted region corresponds to residues 1017-1069 as numbered in the above Cas9 reference sequence, or corresponding amino acid residues thereof.

Exemplary internal fusions base editors are provided in Table 3 below:

TABLE 3
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA-Circular Permutant 136 ins1248 | ISLAY31 |
| IBE032 | TadA-Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

In some embodiments, the Cas9 polypeptide lacks one or more domains selected from the group consisting of: RuvC I, RuvC II, RuvC III, Rec1, Rec2, PI, or HNH domain. In some embodiments, the Cas9 polypeptide lacks a nuclease domain. In some embodiments, the Cas9 polypeptide lacks an HNH domain. In some embodiments, the Cas9 polypeptide lacks a portion of the HNH domain such that the Cas9 polypeptide has reduced or abolished HNH activity. In some embodiments, the Cas9 polypeptide comprises a deletion of the nuclease domain, and the deaminase is inserted to replace the nuclease domain. In some embodiments, the HNH domain is deleted and the deaminase is inserted in its place. In some embodiments, one or more of the RuvC domains is deleted and the deaminase is inserted in its place.

A fusion protein comprising a heterologous polypeptide can be flanked by a N-terminal and a C-terminal fragment of a napDNAbp. In some embodiments, the fusion protein comprises a deaminase flanked by a N-terminal fragment and a C-terminal fragment of a Cas9 polypeptide. The N terminal fragment or the C terminal fragment can bind the target polynucleotide sequence. The C-terminus of the N terminal fragment or the N-terminus of the C terminal fragment can comprise a part of a flexible loop of a Cas9 polypeptide. The C-terminus of the N terminal fragment or the N-terminus of the C terminal fragment can comprise a part of an alpha-helix structure of the Cas9 polypeptide. The N-terminal fragment or the C-terminal fragment can comprise a DNA binding domain. The N-terminal fragment or the C-terminal fragment can comprise a RuvC domain. The N-terminal fragment or the C-terminal fragment can comprise an HNH domain. In some embodiments, neither of the N-terminal fragment and the C-terminal fragment comprises an HNH domain.

In some embodiments, the C-terminus of the N terminal Cas9 fragment comprises an amino acid that is in proximity to a target nucleobase when the fusion protein deaminates the target nucleobase. In some embodiments, the N-terminus of the C terminal Cas9 fragment comprises an amino acid that is in proximity to a target nucleobase when the fusion protein deaminates the target nucleobase. The insertion location of different deaminases can be different in order to have proximity between the target nucleobase and an amino acid in the C-terminus of the N terminal Cas9 fragment or the N-terminus of the C terminal Cas9 fragment. For example, the insertion position of an deaminase can be at an amino acid residue selected from the group consisting of 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

The N-terminal Cas9 fragment of a fusion protein (i.e. the N-terminal Cas9 fragment flanking the deaminase in a fusion protein) can comprise the N-terminus of a Cas9 polypeptide. The N-terminal Cas9 fragment of a fusion protein can comprise a length of at least about: 100, 200, 300, 400, 500, 600, 700, 800, 900, 1000, 1100, 1200, or 1300 amino acids. The N-terminal Cas9 fragment of a fusion protein can comprise a sequence corresponding to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide.

The C-terminal Cas9 fragment of a fusion protein (i.e. the C-terminal Cas9 fragment flanking the deaminase in a fusion protein) can comprise the C-terminus of a Cas9 polypeptide. The C-terminal Cas9 fragment of a fusion protein can comprise a length of at least about: 100, 200, 300, 400, 500, 600, 700, 800, 900, 1000, 1100, 1200, or 1300 amino acids. The C-terminal Cas9 fragment of a fusion protein can comprise a sequence corresponding to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide.

The N-terminal Cas9 fragment and C-terminal Cas9 fragment of a fusion protein taken together may not correspond to a full-length naturally occurring Cas9 polypeptide sequence, for example, as set forth in the above Cas9 reference sequence.

The fusion protein or complex described herein can effect targeted deamination with reduced deamination at non-target sites (e.g., off-target sites), such as reduced genome wide spurious deamination. The fusion protein or complex described herein can effect targeted deamination with reduced bystander deamination at non-target sites. The undesired deamination or off-target deamination can be reduced by at least 30%, at least 40%, at least 50%, at least 60%, at least 70%, at least 80%, at least 90%, at least 95%, or at least 99% compared with, for example, an end terminus fusion protein comprising the deaminase fused to a N terminus or a C terminus of a Cas9 polypeptide. The undesired deamination or off-target deamination can be reduced by at least one-fold, at least two-fold, at least three-fold, at least four-fold, at least five-fold, at least tenfold, at least fifteen fold, at least twenty fold, at least thirty fold, at least forty fold, at least fifty fold, at least 60 fold, at least 70 fold, at least 80 fold, at least 90 fold, or at least hundred fold, compared with, for example, an end terminus fusion protein comprising the deaminase fused to a N terminus or a C terminus of a Cas9 polypeptide.

In some embodiments, the deaminase (e.g., adenosine deaminase, cytidine deaminase, or adenosine deaminase and cytidine deaminase) of the fusion protein or complex deaminates no more than two nucleobases within the range of an R-loop. In some embodiments, the deaminase of the fusion protein or complex deaminates no more than three nucleobases within the range of the R-loop. In some embodiments, the deaminase of the fusion protein or complex deaminates no more than 2, 3, 4, 5, 6, 7, 8, 9, or 10 nucleobases within the range of the R-loop. An R-loop is a three-stranded nucleic acid structure including a DNA:RNA hybrid, a DNA:DNA or an RNA: RNA complementary structure and the associated with single-stranded DNA. As used herein, an R-loop may be formed when a target polynucleotide is contacted with a CRISPR complex or a base editing complex, wherein a portion of a guide polynucleotide, e.g. a guide RNA, hybridizes with and displaces with a portion of a target polynucleotide, e.g. a target DNA. In some embodiments, an R-loop comprises a hybridized region of a spacer sequence and a target DNA complementary sequence. An R-loop region may be of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobase pairs in length. In some embodiments, the R-loop region is about 20 nucleobase pairs in length. It should be understood that, as used herein, an R-loop region is not limited to the target DNA strand that hybridizes with the guide polynucleotide. For example, editing of a target nucleobase within an R-loop region may be to a DNA strand that comprises the complementary strand to a guide RNA, or may be to a DNA strand that is the opposing strand of the strand complementary to the guide RNA. In some embodiments, editing in the region of the R-loop comprises editing a nucleobase on non-complementary strand (protospacer strand) to a guide RNA in a target DNA sequence.

The fusion protein or complex described herein can effect target deamination in an editing window different from canonical base editing. In some embodiments, a target nucleobase is from about 1 to about 20 bases upstream of a PAM sequence in the target polynucleotide sequence. In some embodiments, a target nucleobase is from about 2 to about 12 bases upstream of a PAM sequence in the target polynucleotide sequence. In some embodiments, a target nucleobase is from about 1 to 9 base pairs, about 2 to 10 base pairs, about 3 to 11 base pairs, about 4 to 12 base pairs, about 5 to 13 base pairs, about 6 to 14 base pairs, about 7 to 15 base pairs, about 8 to 16 base pairs, about 9 to 17 base pairs, about 10 to 18 base pairs, about 11 to 19 base pairs, about 12 to 20 base pairs, about 1 to 7 base pairs, about 2 to 8 base pairs, about 3 to 9 base pairs, about 4 to 10 base pairs, about 5 to 11 base pairs, about 6 to 12 base pairs, about 7 to 13 base pairs, about 8 to 14 base pairs, about 9 to 15 base pairs, about 10 to 16 base pairs, about 11 to 17 base pairs, about 12 to 18 base pairs, about 13 to 19 base pairs, about 14 to 20 base pairs, about 1 to 5 base pairs, about 2 to 6 base pairs, about 3 to 7 base pairs, about 4 to 8 base pairs, about 5 to 9 base pairs, about 6 to 10 base pairs, about 7 to 11 base pairs, about 8 to 12 base pairs, about 9 to 13 base pairs, about 10 to 14 base pairs, about 11 to 15 base pairs, about 12 to 16 base pairs, about 13 to 17 base pairs, about 14 to 18 base pairs, about 15 to 19 base pairs, about 16 to 20 base pairs, about 1 to 3 base pairs, about 2 to 4 base pairs, about 3 to 5 base pairs, about 4 to 6 base pairs, about 5 to 7 base pairs, about 6 to 8 base pairs, about 7 to 9 base pairs, about 8 to 10 base pairs, about 9 to 11 base pairs, about 10 to 12 base pairs, about 11 to 13 base pairs, about 12 to 14 base pairs, about 13 to 15 base pairs, about 14 to 16 base pairs, about 15 to 17 base pairs, about 16 to 18 base pairs, about 17 to 19 base pairs, about 18 to 20 base pairs away or upstream of the PAM sequence. In some embodiments, a target nucleobase is about 1, 2, 3, 4, 5, 6, 7, 8, 9, 10, 11, 12, 13, 14, 15, 16, 17, 18, 19, 20, or more base pairs away from or upstream of the PAM sequence. In some embodiments, a target nucleobase is about 1, 2, 3, 4, 5, 6, 7, 8, or 9 base pairs upstream of the PAM sequence. In some embodiments, a target nucleobase is about 2, 3, 4, or 6 base pairs upstream of the PAM sequence.

The fusion protein or complex can comprise more than one heterologous polypeptide. For example, the fusion protein or complex can additionally comprise one or more UGI domains and/or one or more nuclear localization signals. The two or more heterologous domains can be inserted in tandem. The two or more heterologous domains can be inserted at locations such that they are not in tandem in the NapDNAbp.

A fusion protein can comprise a linker between the deaminase and the napDNAbp polypeptide. The linker can be a peptide or a non-peptide linker. For example, the linker can be an XTEN, (GGGS)n (SEQ ID NO: 246), (GGGGS)n (SEQ ID NO: 247), (G)n, (EAAAK)n (SEQ ID NO: 248), (GGS)n, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase, but does not comprise a linker between the C-terminal Cas9 fragment and the deaminase. In some embodiments, the fusion protein comprises a linker between the C-terminal Cas9 fragment and the deaminase, but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

Fusion proteins comprising a heterologous catalytic domain flanked by N- and C-terminal fragments of a Cas12 polypeptide are also useful for base editing in the methods as described herein. Fusion proteins comprising Cas12 and one or more deaminase domains, e.g., adenosine deaminase, or comprising an adenosine deaminase domain flanked by Cas12 sequences are also useful for highly specific and efficient base editing of target sequences. In an embodiment, a chimeric Cas12 fusion protein contains a heterologous catalytic domain (e.g., adenosine deaminase, cytidine deaminase, or adenosine deaminase and cytidine deaminase) inserted within a Cas12 polypeptide. In some embodiments, the fusion protein comprises an adenosine deaminase domain and a cytidine deaminase domain inserted within a Cas12. In some embodiments, an adenosine deaminase is fused within Cas12 and a cytidine deaminase is fused to the C-terminus. In some embodiments, an adenosine deaminase is fused within Cas12 and a cytidine deaminase fused to the N-terminus. In some embodiments, a cytidine deaminase is fused within Cas12 and an adenosine deaminase is fused to the C-terminus. In some embodiments, a cytidine deaminase is fused within Cas12 and an adenosine deaminase fused to the N-terminus. Exemplary structures of a fusion protein with an adenosine deaminase and a cytidine deaminase and a Cas12 are provided as follows:
NH2-[Cas12(adenosine deaminase)]-[cytidine deaminase]-COOH;
NH2-[cytidine deaminase]-[Cas12(adenosine deaminase)]-COOH;
NH2-[Cas12(cytidine deaminase)]-[adenosine deaminase]-COOH; or
NH2-[adenosine deaminase]-[Cas12(cytidine deaminase)]-COOH;

In some embodiments, the “-” used in the general architecture above indicates the optional presence of a linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas12 and a cytidine deaminase is fused to the C-terminus. In some embodiments, a TadA*8 is fused within Cas12 and a cytidine deaminase fused to the N-terminus. In some embodiments, a cytidine deaminase is fused within Cas12 and a TadA*8 is fused to the C-terminus. In some embodiments, a cytidine deaminase is fused within Cas12 and a TadA*8 fused to the N-terminus. Exemplary structures of a fusion protein with a TadA*8 and a cytidine deaminase and a Cas12 are provided as follows:
N-[Cas12(TadA*8)]-[cytidine deaminase]-C;
N-[cytidine deaminase]-[Cas12(TadA*8)]-C;
N-[Cas12(cytidine deaminase)]-[TadA*8]-C; or
N-[TadA*8]-[Cas12(cytidine deaminase)]-C.

In some embodiments, the “-” used in the general architecture above indicates the optional presence of a linker.

In other embodiments, the fusion protein contains one or more catalytic domains. In other embodiments, at least one of the one or more catalytic domains is inserted within the Cas12 polypeptide or is fused at the Cas12 N-terminus or C-terminus. In other embodiments, at least one of the one or more catalytic domains is inserted within a loop, an alpha helix region, an unstructured portion, or a solvent accessible portion of the Cas12 polypeptide. In other embodiments, the Cas12 polypeptide is Cas12a, Cas12b, Cas12c, Cas12d, Cas12e, Cas12g, Cas12h, Cas12i, or Cas12j/CasΦ. In other embodiments, the Cas12 polypeptide has at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b (SEQ ID NO: 254). In other embodiments, the Cas12 polypeptide has at least about 90% amino acid sequence identity to Bacillus hisashii Cas12b (SEQ ID NO: 255), Bacillus thermoamylovorans Cas12b, Bacillus sp. V3-13 Cas12b, or Alicyclobacillus acidiphilus Cas12b. In other embodiments, the Cas12 polypeptide has at least about 95% amino acid sequence identity to Bacillus hisashii Cas12b, Bacillus thermoamylovorans Cas12b (SEQ ID NO: 256), Bacillus sp. V3-13 Cas12b (SEQ ID NO: 257), or Alicyclobacillus acidiphilus Cas12b. In other embodiments, the Cas12 polypeptide contains or consists essentially of a fragment of Bacillus hisashii Cas12b, Bacillus thermoamylovorans Cas12b, Bacillus sp. V3-13 Cas12b, or Alicyclobacillus acidiphilus Cas12b. In embodiments, the Cas12 polypeptide contains BvCas12b (V4), which in some embodiments is expressed as 5′ mRNA Cap---5′ UTR---bhCas12b---STOP sequence---3′ UTR---120polyA tail (SEQ ID NOs: 258-260).

In other embodiments, the catalytic domain is inserted between amino acid positions 153-154, 255-256, 306-307, 980-981, 1019-1020, 534-535, 604-605, or 344-345 of BhCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P153 and S154 of BhCas12b. In other embodiments, the catalytic domain is inserted between amino acids K255 and E256 of BhCas12b. In other embodiments, the catalytic domain is inserted between amino acids D980 and G981 of BhCas12b. In other embodiments, the catalytic domain is inserted between amino acids K1019 and L1020 of BhCas12b. In other embodiments, the catalytic domain is inserted between amino acids F534 and P535 of BhCas12b. In other embodiments, the catalytic domain is inserted between amino acids K604 and G605 of BhCas12b. In other embodiments, the catalytic domain is inserted between amino acids H344 and F345 of BhCas12b. In other embodiments, catalytic domain is inserted between amino acid positions 147 and 148, 248 and 249, 299 and 300, 991 and 992, or 1031 and 1032 of BvCas12b or a corresponding amino acid residue of Cas12a, Cas12c, Cas12d, Cas12e, Cas12g, Cas12h, Cas12i, or Cas12j/Cas4. In other embodiments, the catalytic domain is inserted between amino acids P147 and D148 of BvCas12b. In other embodiments, the catalytic domain is inserted between amino acids G248 and G249 of BvCas12b. In other embodiments, the catalytic domain is inserted between amino acids P299 and E300 of BvCas12b. In other embodiments, the catalytic domain is inserted between amino acids G991 and E992 of BvCas12b. In other embodiments, the catalytic domain is inserted between amino acids K1031 and M1032 of BvCas12b. In other embodiments, the catalytic domain is inserted between amino acid positions 157 and 158, 258 and 259, 310 and 311, 1008 and 1009, or 1044 and 1045 of AaCas12b or a corresponding amino acid residue of Cas12a, Cas12c, Cas12d, Cas12e, Cas12g, Cas12h, Cas12i, or Cas12j/Cas4. In other embodiments, the catalytic domain is inserted between amino acids P157 and G158 of AaCas12b. In other embodiments, the catalytic domain is inserted between amino acids V258 and G259 of AaCas12b. In other embodiments, the catalytic domain is inserted between amino acids D310 and P311 of AaCas12b. In other embodiments, the catalytic domain is inserted between amino acids G1008 and E1009 of AaCas12b. In other embodiments, the catalytic domain is inserted between amino acids G1044 and K1045 at of AaCas12b.

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:
ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein or complex further contains a tag (e.g., an influenza hemagglutinin tag).

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4 below.

TABLE 4
Insertion loci in Cas12b proteins

| BhCas 12b | Insertion site | Inserted between aa |
|---|---|---|
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | Insertion site | Inserted between aa |
| position 1 | 147 | PD |
| position 2 | 248 | GG |
| position 3 | 299 | PE |
| position 4 | 991 | GE |
| position 5 | 1031 | KM |
| AaCas12b | Insertion site | Inserted between aa |
| position 1 | 157 | PG |
| position 2 | 258 | VG |
| position 3 | 310 | DP |
| position 4 | 1008 | GE |
| position 5 | 1044 | GK |

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

In some embodiments, adenosine base editors were generated to insert TadA or variants thereof into the Cas9 polypeptide at the identified positions.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA). In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion (e.g., a functional portion) of adenosine deaminase acting on RNA (ADAR, e.g., ADAR1 or ADAR2) or tRNA (ADAT). A base editor comprising an adenosine deaminase domain can also be capable of deaminating an A nucleobase of a DNA polynucleotide. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises a D108X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in a TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in a TadA reference sequence, or a corresponding mutation in another adenosine deaminase (e.g. ecTadA).

In some embodiments, the adenosine deaminase comprises a E155X mutation in a TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in a TadA reference sequence, or a corresponding mutation in another adenosine deaminase (e.g., ecTadA).

In some embodiments, the adenosine deaminase comprises a D147X mutation in a TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in a TadA reference sequence, or a corresponding mutation in another adenosine deaminase (e.g., ecTadA).

In some embodiments, the adenosine deaminase comprises an A106X, E155X, or D147X, mutation in a TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

It should also be appreciated that any of the mutations provided herein may be made individually or in any combination in ecTadA or another adenosine deaminase. For example, an adenosine deaminase may contain a D108N, a A106V, a E155V, and/or a D147Y mutation in TadA reference sequence, or a corresponding mutation in another adenosine deaminase (e.g., ecTadA). In some embodiments, an adenosine deaminase comprises the following group of mutations (groups of mutations are separated by a “;”) in a TadA reference sequence (e.g., TadA*7.10 (SEQ ID NO:1), or corresponding mutations in another adenosine deaminase: D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein may be made in an adenosine deaminase (e.g., ecTadA).

In some embodiments, the adenosine deaminase comprises a combination of mutations in a TadA reference sequence (e.g., TadA*7.10 (SEQ ID NO: 1)), or corresponding mutations in another adenosine deaminase: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N.

In some embodiments, the adenosine deaminase comprises one or more of a H8X, T17X, L18X, W23X, L34X, W45X, R51X, A56X, E59X, E85X, M94X, I95X, V102X, F104X, A106X, R107X, D108X, K110X, M118X, N127X, A138X, F149X, M151X, R153X, Q154X, I156X, and/or K157X mutation in a TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, I95L, V102A, F104L, A106V, R107C, or R107H, or R107P, D108G, or D108N, or D108V, or D108A, or D108Y, K110I, M118K, N127S, A138V, F149Y, M151V, R153C, Q154L, 1156D, and/or K157R mutation in a TadA reference sequence, or one or more corresponding mutations in another adenosine deaminase.

In some embodiments, the adenosine deaminase comprises one or more of a H8X, D108X, and/or N127X mutation in a TadA reference sequence, or one or more corresponding mutations in another adenosine deaminase, where X indicates the presence of any amino acid. In some embodiments, the adenosine deaminase comprises one or more of a H8Y, D108N, and/or N127S mutation in a TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in a TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in a TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in a TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in a TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in a TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8X, AT06X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of H8X, R126X, L68X, D108X, N127X, D147X, and E155X in a TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in a TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in a TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M61I, M70V, D108N, N127S, Q154R, E155G and Q163H in a TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in a TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in a TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in a TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, A109T, N127S, and E155G in a TadA reference sequence, or a corresponding mutation or mutations in another adenosine deaminase (e.g., ecTadA).

In some embodiments, the adenosine deaminase comprises one or more of the or one or more corresponding mutations in another adenosine deaminase. In some embodiments, the adenosine deaminase comprises a D108N, D108G, or D108V mutation in a TadA reference sequence, or corresponding mutations in another adenosine deaminase. In some embodiments, the adenosine deaminase comprises a A106V and D108N mutation in a TadA reference sequence, or corresponding mutations in another adenosine deaminase. In some embodiments, the adenosine deaminase comprises R107C and D108N mutations in a TadA reference sequence, or corresponding mutations in another adenosine deaminase. In some embodiments, the adenosine deaminase comprises a H8Y, D108N, N127S, D147Y, and Q154H mutation in a TadA reference sequence, or corresponding mutations in another adenosine deaminase. In some embodiments, the adenosine deaminase comprises a H8Y, D108N, N127S, D147Y, and E155V mutation in a TadA reference sequence, or corresponding mutations in another adenosine deaminase. In some embodiments, the adenosine deaminase comprises a D108N, D147Y, and E155V mutation in a TadA reference sequence, or corresponding mutations in another adenosine deaminase. In some embodiments, the adenosine deaminase comprises a H8Y, D108N, and N127S mutation in a TadA reference sequence, or corresponding mutations in another adenosine deaminase. In some embodiments, the adenosine deaminase comprises a A106V, D108N, D147Y, and E155V mutation in a TadA reference sequence, or corresponding mutations in another adenosine deaminase (e.g., ecTadA).

In some embodiments, the adenosine deaminase comprises one or more of S2X, H8X, I49X, L84X, H123X, N127X, I156X, and/or K160X mutation in a TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, I49F, L84F, H123Y, N127S, I156F, and/or K160S mutation in a TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in a TadA reference sequence, or a corresponding mutation in another adenosine deaminase (e.g., ecTadA).

In some embodiments, the adenosine deaminase comprises an H123X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in a TadA reference sequence, or a corresponding mutation in another adenosine deaminase.

In some embodiments, the adenosine deaminase comprises an I156X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an I156F mutation in a TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in a TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in a TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in a TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in a TadA reference sequence, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in a TadA reference sequence.

In some embodiments, the adenosine deaminase comprises one, two, three, four, or five mutations selected from the group consisting of H8Y, A106T, D108N, N127S, and K160S in a TadA reference sequence, or a corresponding mutation or mutations in another adenosine deaminase.

In some embodiments, the adenosine deaminase comprises one or more of a E25X, R26X, R107X, A142X, and/or A143X mutation in a TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q, and/or A143R mutation in a TadA reference sequence, or one or more corresponding mutations in another adenosine deaminase. In some embodiments, the adenosine deaminase comprises one or more of the mutations described herein corresponding to a TadA reference sequence, or one or more corresponding mutations in another adenosine deaminase.

In some embodiments, the adenosine deaminase comprises an E25X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in a TadA reference sequence, or a corresponding mutation in another adenosine deaminase (e.g., ecTadA).

In some embodiments, the adenosine deaminase comprises an R26X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in a TadA reference sequence, or a corresponding mutation in another adenosine deaminase (e.g., ecTadA).

In some embodiments, the adenosine deaminase comprises an R107X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R107K, RT07A, RT07N, RT07W, RT07H, or RT07S mutation in a TadA reference sequence, or a corresponding mutation in another adenosine deaminase (e.g., ecTadA).

In some embodiments, the adenosine deaminase comprises an A142X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in a TadA reference sequence, or a corresponding mutation in another adenosine deaminase (e.g., ecTadA).

In some embodiments, the adenosine deaminase comprises an A143X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in a TadA reference sequence, or a corresponding mutation in another adenosine deaminase (e.g., ecTadA).

In some embodiments, the adenosine deaminase comprises one or more of a H36X, N37X, P48X, I49X, R51X, M70X, N72X, D77X, E134X, S146X, Q154X, K157X, and/or K161X mutation in a TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, I49V, R51H, R51L, M70L, N72S, D77G, E134G, S146R, S146C, Q154H, K157N, and/or K161T mutation in a TadA reference sequence, or one or more corresponding mutations in another adenosine deaminase (e.g., ecTadA).

In some embodiments, the adenosine deaminase comprises an H36X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in a TadA reference sequence, or a corresponding mutation in another adenosine deaminase.

In some embodiments, the adenosine deaminase comprises an N37X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in a TadA reference sequence, or a corresponding mutation in another adenosine deaminase.

In some embodiments, the adenosine deaminase comprises an P48X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in a TadA reference sequence, or a corresponding mutation in another adenosine deaminase.

In some embodiments, the adenosine deaminase comprises an R51X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in a TadA reference sequence, or a corresponding mutation in another adenosine deaminase.

In some embodiments, the adenosine deaminase comprises an S146X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in a TadA reference sequence, or a corresponding mutation in another adenosine deaminase.

In some embodiments, the adenosine deaminase comprises an K157X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in a TadA reference sequence, or a corresponding mutation in another adenosine deaminase.

In some embodiments, the adenosine deaminase comprises an P48X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in a TadA reference sequence, or a corresponding mutation in another adenosine deaminase.

In some embodiments, the adenosine deaminase comprises an A142X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in a TadA reference sequence, or a corresponding mutation in another adenosine deaminase.

In some embodiments, the adenosine deaminase comprises an W23X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in a TadA reference sequence, or a corresponding mutation in another adenosine deaminase.

In some embodiments, the adenosine deaminase comprises an R152X mutation in a TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in a TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to a TadA reference sequence, where each mutation of a combination is separated by a “_” and each combination of mutations is between parentheses:
(A106V_D108N),
(R107C_D108N),
(H8Y_D108N_N127S_D147Y_Q154H),
(H8Y_D108N_N127S_D147Y_E155V),
(D108N_D147Y_E155V),
(H8Y_D108N_N127S),
(H8Y_D108N_N127S_D147Y_Q154H),
(A106V_D108N_D147Y_E155V),
(D108Q_D147Y_E155V),
(D108M_D147Y_E155V),
(D108L_D147Y_E155V),
(D108K_D147Y_E155V),
(D108I_D147Y_E155V),
(D108F_D147Y_E155V),
(A106V_D108N_D147Y),
(A106V_D108M_D147Y_E155V),
(E59A_A106V_D108N_D147Y_E155V),
(E59A cat dead_A106V_D108N_D147Y_E155V),
(L84F_A106V_D108N_H123Y_D147Y_E155V_I156Y),
(L84F_A106V_D108N_H123Y_D147Y_E155V_I156F),
(D103A_D104N),
(G22P_D103A_D104N),
(D103A_D104N_S138A),
(R26G_L84F_A106V_R107H_D108N_H123Y_A142N_A143D_D147Y_E155V_I156F),
(E25G_R26G_L84F_A106V_R107H_D108N_H123Y_A142N_A143D_D147Y_E155V_I156F),
(E25D_R26G_L84F_A106V_R107K_D108N_H123Y_A142N_A143G_D147Y_E155V_I156F),
(R26Q_L84F_A106V_D108N_H123Y_A142N_D147Y_E155V_I156F),
(E25M_R26G_L84F_A106V_R107P_D108N_H123Y_A142N_A143D_D147Y_E155V_I156F),
(R26C_L84F_A106V_R107H_D108N_H123Y_A142N_D147Y_E155V_I156F),
(L84F_A106V_D108N_H123Y_A142N_A143L_D147Y_E155V_I156F),
(R26G_L84F_A106V_D108N_H123Y_A142N_D147Y_E155V_I156F),
(E25A_R26G_L84F_A106V_R107N_D108N_H123Y_A142N_A143E_D147Y_E155V_I156F),
(R26G_L84F_A106V_R107H_D108N_H123Y_A142N_A143D_D147Y_E155V_I156F),
(A106V_D108N_A142N_D147Y_E155V),
(R26G_A106V_D108N_A142N_D147Y_E155V),
(E25D_R26G_A106V_R107K_D108N_A142N_A143G_D147Y_E155V),
(R26G_A106V_D108N_R107H_A142N_A143D_D147Y_E155V),
(E25D_R26G_A106V_D108N_A142N_D147Y_E155V),
(A106V_R107K_D108N_A142N_D147Y_E155V),
(A106V_D108N_A142N_A143G_D147Y_E155V),
(A106V_D108N_A142N_A143L_D147Y_E155V),
(H36L_R51L_L84F_A106V_D108N_H123Y_S146C_D147Y_E155V_I156F_K157N),
(N37T_P48T_M70L_L84F_A106V_D108N_H123Y_D147Y_149V_E155V_1156F),
(N37S_L84F_A106V_D108N_H123Y_D147Y_E155V_I156F_K161T),
(H36L_L84F_A106V_D108N_H123Y_D147Y_Q154H_E155V_I156F),
(N72S_L84F_A106V_D108N_H123Y_S146R_D147Y_E155V_I156F),
(H36L_P48L_L84F_A106V_D108N_H123Y_E134G_D147Y_E155V_I156F),
(H36L_L84F_A106V_D108N_H123Y_D147Y_E155V_I156F_K157N)
(H36L_L84F_A106V_D108N_H123Y_S146C_D147Y_E155V_I156F),
(L84F_A106V_D108N_H123Y_S146R_D147Y_E155V_I156F_K161T),
(N37S_R51H_D77G_L84F_A106V_D108N_H123Y_D147Y_E155V_I156F),
(R51L_L84F_A106V_D108N_H123Y_D147Y_E155V_I156F_K157N),
(D24G_Q71R_L84F_H96L_A106V_D108N_H123Y_D147Y_E155V_I156F_K160E),
(H36L_G67V_L84F_A106V_D108N_H123Y_S146T_D147Y_E155V_I156F),
(Q71L_L84F_A106V_D108N_H123Y_L137M_A143E_D147Y_E155V_I156F),
(E25G_L84F_A106V_D108N_H123Y_D147Y_E155V_I156F_Q159L),
(L84F_A91T_F104I_A106V_D108N_H123Y_D147Y_E155V_I156F),
(N72D_L84F_A106V_D108N_H123Y_G125A_D147Y_E155V_I156F),
(P48S_L84F_S97C_A106V_D108N_H123Y_D147Y_E155V_I156F),
(W23G_L84F_A106V_D108N_H123Y_D147Y_E155V_I156F),
(D24G_P48L_Q71R_L84F_A106V_D108N_H123Y_D147Y_E155V_I156F_Q159L),
(L84F_A106V_D108N_H123Y_A142N_D147Y_E155V_I156F),
(H36L_R51L_L84F_A106V_D108N_H123Y_A142N_S146C_D147Y_E155V_I156F_K157N),
(N37S_L84F_A106V_D108N_H123Y_A142N_D147Y_E155V_I156F_K161T),
(L84F_A106V_D108N_D147Y_E155V_I156F),
(R51L_L84F_A106V_D108N_H123Y_S146C_D147Y_E155V_I156F_K157N_K161T),
(L84F_A106V_D108N_H123Y_S146C_D147Y_E155V_I156F_K161T),
(L84F_A106V_D108N_H123Y_S146C_D147Y_E155V_I156F_K157N_K160E_K161T),
(L84F_A106V_D108N_H123Y_S146C_D147Y_E155V_1156F_K157N_K160E),
(R74Q_L84F_A106V_D108N_H123Y_D147Y_E155V_I156F),
(R74A_L84F_A106V_D108N_H123Y_D147Y_E155V_I156F),
(L84F_A106V_D108N_H123Y_D147Y_E155V_I156F),
(R74Q_L84F_A106V_D108N_H123Y_D147Y_E155V_I156F),
(L84F_R98Q_A106V_D108N_H123Y_D147Y_E155V_I156F),
(L84F_A106V_D108N_H123Y_R129Q_D147Y_E155V_I156F),
(P48S_L84F_A106V_D108N_H123Y_A142N_D147Y_E155V_I156F),
(P48S_A142N),
(P48T_I49V_L84F_A106V_D108N_H123Y_A142N_D147Y_E155V_I156F_L157N),
(P48T_I49V_A142N),
(H36L_P48S_R51L_L84F_A106V_D108N_H123Y_S146C_D147Y_E155V_I156F_K157N),
(H36L_P48S_R51L_L84F_A106V_D108N_H123Y_S146C_A142N_D147Y_E155V_I156F
(H36L_P48T_I49V_R51L_L84F_A106V_D108N_H123Y_S146C_D147Y_E155V_I156F_K157N),
(H36L_P48T_I49V_R51L_L84F_A106V_D108N_H123Y_A142N_S146C_D147Y_E155V_I156F_K157N),
(H36L_P48A_R51L_L84F_A106V_D108N_H123Y_S146C_D147Y_E155V_I156F_K157N),
(H36L_P48A_R51L_L84F_A106V_D108N_H123Y_A142N_S146C_D147Y_E155V_I156F_K157N),
(H36L_P48A_R51L_L84F_A106V_D108N_H123Y_S146C_A142N_D147Y_E155V_I156F_K157N),
(W23L_H36L_P48A_R51L_L84F_A106V_D108N_H123Y_S146C_D147Y_E155V_I156F_K157N),
(W23R_H36L_P48A_R51L_L84F_A106V_D108N_H123Y_S146C_D147Y_E155V_I156F_K157N),
(W23L_H36L_P48A_R51L_L84F_A106V_D108N_H123Y_S146R_D147Y_E155V_I156F_K161T),
(H36L_P48A_R51L_L84F_A106V_D108N_H123Y_S146C_D147Y_R152H_E155V_I156F_K157N),
(H36L_P48A_R51L_L84F_A106V_D108N_H123Y_S146C_D147Y_R152P_E155V_I156F_K157N),
(W23L_H36L_P48A_R51L_L84F_A106V_D108N_H123Y_S146C_D147Y_R152P_E155V_I156F_K157N),
(W23L_H36L_P48A_R51L_L84F_A106V_D108N_H123Y_A142A_S146C_D147Y_E155V_I156F_K157N),
(W23L_H36L_P48A_R51L_L84F_A106V_D108N_H123Y_A142A_S146C_D147Y_R152P_E155V_I156F_K157N),
(W23L_H36L_P48A_R51L_L84F_A106V_D108N_H123Y_S146R_D147Y_E155V_I156F_K161T),
(W23R_H36L_P48A_R51L_L84F_A106V_D108N_H123Y_S146C_D147Y_R152P_E155V_I156F_K157N), (H36L_P48A_R51L_L84F_A106V_D108N_H123Y_A142N_S146C_D147Y_R152P_E155V_I156F_K157N).

In some embodiments, the TadA deaminase is a TadA variant. In some embodiments, the TadA variant is TadA*7.10. In particular embodiments, the fusion proteins or complexes comprise a single TadA*7.10 domain (e.g., provided as a monomer). In other embodiments, the fusion protein comprises TadA*7.10 and TadA(wt), which are capable of forming heterodimers.

In one embodiment, a fusion protein of the present disclosure comprises a wild-type TadA linked to TadA*7.10, which is linked to Cas9 nickase.

In some embodiments, TadA*7.10 comprises at least one alteration. In some embodiments, the adenosine deaminase comprises an alteration in the following sequence:

TadA*7.10
(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD

In some embodiments, TadA*7.10 comprises an alteration at amino acid 82 and/or 166.

In particular embodiments, TadA*7.10 comprises one or more of the following alterations: Y147T, Y147R, Q154S, Y123H, V82S, T166R, and/or Q154R. In other embodiments, a variant of TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R.

In some embodiments, a variant of TadA*7.10 comprises one or more of alterations selected from the group of L36H, I76Y, V82G, Y147T, Y147D, F149Y, Q154S, N157K, and/or D167N. In some embodiments, a variant of TadA*7.10 comprises V82G, Y147T/D, Q154S, and one or more of L36H, I76Y, F149Y, N157K, and D167N. In other embodiments, a variant of TadA*7.10 comprises a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N.

In some embodiments, an adenosine deaminase variant (e.g., TadA*8) comprises a deletion. In some embodiments, an adenosine deaminase variant comprises a deletion of the C terminus. In particular embodiments, an adenosine deaminase variant comprises a deletion of the C terminus beginning at residue 149, 150, 151, 152, 153, 154, 155, 156, and 157, relative to a TadA reference sequence (e.g., TadA*7.10 (SEQ ID NO: 1), the TadA reference sequence, or a corresponding mutation in another TadA.

In other embodiments, an adenosine deaminase variant (e.g., TadA*8) is a monomer comprising one or more of the following alterations: Y147T, Y147R, Q154S, Y123H, V82S, T166R, and/or Q154R, relative to a TadA reference sequence (e.g., TadA*7.10 (SEQ ID NO: 1), or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1), or a corresponding mutation in another TadA.

In other embodiments, the adenosine deaminase variant is a homodimer comprising two adenosine deaminase domains (e.g., TadA*8) each having one or more of the following alterations Y147T, Y147R, Q154S, Y123H, V82S, T166R, and/or Q154R, relative to a TadA reference sequence (e.g., TadA*7.10 (SEQ ID NO: 1), or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1), or a corresponding mutation in another TadA.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to a TadA reference sequence (e.g., TadA*7.10 (SEQ ID NO: 1), or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T1661+D167N; V88A+A109S+T111R+D119N+H122N+F149Y+T1661+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1), or a corresponding mutation in another TadA.

In some embodiments, an adenosine deaminase variant (e.g., MSP828) is a monomer comprising one or more of the following alterations L36H, I76Y, V82G, Y147T, Y147D, F149Y, Q154S, N157K, and/or D167N, relative to a TadA reference sequence (e.g., TadA*7.10 (SEQ ID NO: 1), or a corresponding mutation in another TadA. In some embodiments, an adenosine deaminase variant (e.g., MSP828) is a monomer comprising V82G, Y147T/D, Q154S, and one or more of L36H, I76Y, F149Y, N157K, and D167N, relative to a TadA reference sequence (e.g., TadA*7.10 (SEQ ID NO: 1), or a corresponding mutation in another TadA. In other embodiments, the adenosine deaminase variant (TadA variant) is a monomer comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to a TadA reference sequence (e.g., TadA*7.10 (SEQ ID NO: 1), or a corresponding mutation in another TadA.

In other embodiments, the adenosine deaminase variant is a heterodimer of a wild-type adenosine deaminase domain and an adenosine deaminase variant domain (e.g., TadA*8) comprising one or more of the following alterations Y147T, Y147R, Q154S, Y123H, V82S, T166R, and/or Q154R, relative to a TadA reference sequence (e.g., TadA*7.10 (SEQ ID NO: 1), or a corresponding mutation in another TadA. In other embodiments, the adenosine deaminase variant is a heterodimer of a wild-type adenosine deaminase domain and an adenosine deaminase variant domain (e.g., TadA*8) comprising a combination of alterations selected from the group of Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative a TadA reference sequence (e.g., TadA*7.10 (SEQ ID NO: 1), or a corresponding mutation in another TadA.

In other embodiments, a base editor of the disclosure comprising an adenosine deaminase variant (e.g., TadA*8) homodimer comprising two adenosine deaminase domains (e.g., TadA*8) each having one or more of the following alterations R26C, V88A, A109S, T111R, D119N, H122N, Y147D, F149Y, T166I and/or D167N, relative to a TadA reference sequence (e.g., TadA*7.10 (SEQ ID NO: 1), or a corresponding mutation in another TadA. In other embodiments, the adenosine deaminase variant is a homodimer comprising two adenosine deaminase domains (e.g., TadA*8) each having a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1), or a corresponding mutation in another TadA.

In some embodiments, an adenosine deaminase variant is a homodimer comprising two adenosine deaminase domains (e.g., TadA*7.10) each having one or more of the following alterations L36H, I76Y, V82G, Y147T, Y147D, F149Y, Q154S, N157K, and/or D167N, relative to a TadA reference sequence (e.g., TadA*7.10 (SEQ ID NO: 1), or a corresponding mutation in another TadA. In some embodiments, an adenosine deaminase variant is a homodimer comprising two adenosine deaminase variant domains (e.g., MSP828) each having the following alterations V82G, Y147T/D, Q154S, and one or more of L36H, I76Y, F149Y, N157K, and D167N, relative to a TadA reference sequence (e.g., TadA*7.10 (SEQ ID NO: 1), or a corresponding mutation in another TadA. In other embodiments, the adenosine deaminase variant is a homodimer comprising two adenosine deaminase domains (e.g., TadA*7.10) each having a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to a TadA reference sequence (e.g., TadA*7.10 (SEQ ID NO: 1), or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to a TadA reference sequence (e.g., TadA*7.10 (SEQ ID NO: 1), or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1), or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to a TadA reference sequence (e.g., TadA*7.10 (SEQ ID NO: 1), or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T1661+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1), or a corresponding mutation in another TadA.

In other embodiments, the adenosine deaminase variant is a heterodimer of a wild-type adenosine deaminase domain and an adenosine deaminase variant domain (e.g., TadA*7.10) comprising one or more of the following alterations L36H, I76Y, V82G, Y147T, Y147D, F149Y, Q154S, N157K, and/or D167N, relative to a TadA reference sequence (e.g., TadA*7.10 (SEQ ID NO: 1), or a corresponding mutation in another TadA. In some embodiments, an adenosine deaminase variant is a heterodimer comprising a wild-type adenosine deaminase domain and an adenosine deaminase variant domain (e.g., MSP828) having the following alterations V82G, Y147T/D, Q154S, and one or more of L36H, I76Y, F149Y, N157K, and D167N, relative to a TadA reference sequence (e.g., TadA*7.10 (SEQ ID NO: 1), or a corresponding mutation in another TadA. In other embodiments, the adenosine deaminase variant is a heterodimer of a wild-type adenosine deaminase domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; 176Y+V82G+Y147D+F149Y+Q154S+D167N; L36H+I76Y+V82G+Y147D+F149Y+Q154S+N157K+D167N, relative to a TadA reference sequence (e.g., TadA*7.10 (SEQ ID NO: 1), or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to a TadA reference sequence (e.g., TadA*7.10 (SEQ ID NO: 1), or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to a TadA reference sequence (e.g., TadA*7.10 (SEQ ID NO: 1), or a corresponding mutation in another TadA.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, an adenosine deaminase is a TadA*8. In one embodiment, an adenosine deaminase is a TadA*8 that comprises or consists essentially of the following sequence, or a fragment thereof having adenosine deaminase activity:

(SEQ ID NO: 316)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCT
FFRMPRQVFNAQKKAQSSTD

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to a TadA reference sequence (e.g., TadA*7.10 (SEQ ID NO: 1), or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T1661+D167N; V88A+A109S+T111R+D119N+H122N+F149Y+T1661+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1), or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to a TadA reference sequence (e.g., TadA*7.10 (SEQ ID NO: 1), or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of R26C+A109S+T111R+D119N+H122N+Y147D+F149Y+T1661+D167N; V88A+A109S+T111R+D119N+H122N+F149Y+T1661+D167N; R26C+A109S+T111R+D119N+H122N+F149Y+T1661+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1), or a corresponding mutation in another TadA.

In other embodiments, a base editor comprises a heterodimer of a TadA*7.10 domain and an adenosine deaminase variant domain (e.g., TadA*8) comprising one or more of the following alterations R26C, V88A, A109S, T111R, D119N, H122N, Y147D, F149Y, T166I and/or D167N, relative to a TadA reference sequence (e.g., TadA*7.10 (SEQ ID NO: 1), or a corresponding mutation in another TadA. In other embodiments, the base editor comprises a heterodimer of a TadA*7.10 domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to a TadA reference sequence (e.g., TadA*7.10 (SEQ ID NO: 1), or a corresponding mutation in another TadA.

In some embodiments, the TadA*8 is a variant as shown in Table 5. Table 5 shows certain amino acid position numbers in the TadA amino acid sequence and the amino acids present in those positions in the TadA-7.10 adenosine deaminase. Table 5 also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e.

In other embodiments, the adenosine deaminase variant is a heterodimer of a TadA*7.10 domain and an adenosine deaminase variant domain (e.g., TadA*7.10) comprising one or more of the following alterations L36H, I76Y, V82G, Y147T, Y147D, F149Y, Q154S, N157K, and/or D167N, relative to a TadA reference sequence (e.g., TadA*7.10 (SEQ ID NO: 1), or a corresponding mutation in another TadA. In some embodiments, an adenosine deaminase variant is a heterodimer comprising a TadA*7.10 domain and an adenosine deaminase variant domain (e.g., MSP828) having the following alterations V82G, Y147T/D, Q154S, and one or more of L36H, I76Y, F149Y, N157K, and D167N, relative to a TadA reference sequence (e.g., TadA*7.10 (SEQ ID NO: 1), or a corresponding mutation in another TadA. In other embodiments, the adenosine deaminase variant is a heterodimer of a TadA*7.10 domain and an adenosine deaminase variant domain (e.g., TadA*7.10) comprising a combination of alterations selected from the group of: V82G+Y147T+Q154S; 176Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; 176Y+V82G+Y147D+F149Y+Q154S+D167N; L36H+176Y+V82G+Y147D+F149Y+Q154S+N157K+D167N, relative to a TadA reference sequence (e.g., TadA*7.10 (SEQ ID NO: 1), or a corresponding mutation in another TadA.

TABLE 5
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5.1. Table 5.1 shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5.1
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In one embodiment, a fusion protein or complex of the present disclosure comprises a wild-type TadA is linked to an adenosine deaminase variant described herein (e.g., TadA*8), which is linked to Cas9 nickase. In particular embodiments, the fusion proteins or complexes comprise a single TadA*8 domain (e.g., provided as a monomer). In other embodiments, the fusion protein or complex comprises TadA*8 and TadA(wt), which are capable of forming heterodimers.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In particular embodiments, a TadA*8 comprises one or more mutations at any of the following positions shown in bold. In other embodiments, a TadA*8 comprises one or more mutations at any of the positions shown with underlining:

| (SEQ ID NO: 1) | |
|---|---|
| MSEVEFSHEY WMRHALTLAK RARDEREVPV GAVLVLNNRV IGEGWNRAIG | 50 |
| LHDPTAHAEI MALRQGGLVM QNYRLIDATL YVTFEPCVMC AGAMIHSRIG | 100 |
| RVVFGVRNAK TGAAGSLMDV LHYPGMNHRV EITEGILADE CAALLCYFFR | 150 |
| MPRQVFNAQK KAQSSTD | |

For example, the TadA*8 comprises alterations at amino acid position 82 and/or 166 (e.g., V82S, T166R) alone or in combination with any one or more of the following Y147T, Y147R, Q154S, Y123H, and/or Q154R, relative to a TadA reference sequence (e.g., TadA*7.10 (SEQ ID NO: 1)), or a corresponding mutation in another TadA. In particular embodiments, a combination of alterations is selected from the group of: Y147T+Q154R; Y147T+Q154S; Y147R+Q154S; V82S+Q154S; V82S+Y147R; V82S+Q154R; V82S+Y123H; 176Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and 176Y+V82S+Y123H+Y147R+Q154R, relative to a TadA reference sequence (e.g., TadA*7.10 (SEQ ID NO: 1)), or a corresponding mutation in another TadA.

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein or complex of the present disclosure comprises a wild-type TadA is linked to an adenosine deaminase variant described herein (e.g., TadA*8), which is linked to Cas9 nickase. In particular embodiments, the fusion proteins or complexes comprise a single TadA*8 domain (e.g., provided as a monomer). In other embodiments, the base editor comprises TadA*8 and TadA(wt), which are capable of forming heterodimers.

In particular embodiments, the fusion proteins or complexes comprise a single (e.g., provided as a monomer) TadA*8. In some embodiments, the TadA*8 is linked to a Cas9 nickase. In some embodiments, the fusion proteins or complexes of the present disclosure comprise as a heterodimer of a wild-type TadA (TadA(wt)) linked to a TadA*8. In other embodiments, the fusion proteins or complexes of the present disclosure comprise as a heterodimer of a TadA*7.10 linked to a TadA*8. In some embodiments, the base editor is ABE8 comprising a TadA*8 variant monomer. In some embodiments, the base editor is ABE8 comprising a heterodimer of a TadA*8 and a TadA(wt). In some embodiments, the base editor is ABE8 comprising a heterodimer of a TadA*8 and TadA*7.10. In some embodiments, the base editor is ABE8 comprising a heterodimer of a TadA*8. In some embodiments, the TadA*8 is selected from Table 5, 11, or 12. In some embodiments, the ABE8 is selected from Table 11, 12, or 14.

In some embodiments, the adenosine deaminase is a TadA*9 variant. In some embodiments, the adenosine deaminase is a TadA*9 variant selected from the variants described below and with reference to the following sequence (termed TadA*7.10):

(SEQ ID NO: 1)
MSEVEFSHEY WMRHALTLAK RARDEREVPV GAVLVLNNRV
IGEGWNRAIG LHDPTAHAEI MALRQGGLVM QNYRLIDATL
YVTFEPCVMC AGAMIHSRIG RVVFGVRNAK TGAAGSLMDV
LHYPGMNHRV EITEGILADE CAALLCYFFR MPRQVFNAQK
KAQSSTD

In some embodiments, an adenosine deaminase comprises one or more of the following alterations: R21N, R23H, E25F, N38G, L51W, P54C, M70V, Q71M, N72K, Y73S, V82T, M94V, P124W, T133K, D139L, D139M, C146R, and A158K. The one or more alternations are shown in the sequence above in underlining and bold font.

In some embodiments, an adenosine deaminase comprises one or more of the following combinations of alterations: V82S+Q154R+Y147R; V82S+Q154R+Y123H; V82S+Q154R+Y147R+Y123H; Q154R+Y147R+Y123H+I76Y+V82S; V82S+I76Y; V82S+Y147R; V82S+Y147R+Y123H; V82S+Q154R+Y123H; Q154R+Y147R+Y123H+I76Y; V82S+Y147R; V82S+Y147R+Y123H; V82S+Q154R+Y123H; V82S+Q154R+Y147R; V82S+Q154R+Y147R; Q154R+Y147R+Y123H+I76Y; Q154R+Y147R+Y123H+I76Y+V82S; I76Y_V82S_Y123H_Y147R_Q154R; Y147R+Q154R+H123H; and V82S+Q154R.

In some embodiments, an adenosine deaminase comprises one or more of the following combinations of alterations: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; and M70V+V82S+M94V+Y123H+Y147R+Q154R In some embodiments, an adenosine deaminase comprises one or more of the following combinations of alterations: Q71M+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; 176Y+V82S+Y123H+D139M+Y147R+Q154R; Y73S+I76Y+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; 176Y+V82S+Y123H+D139M+Y147R+Q154R; Y73S+I76Y+V82S+Y123H+Y147R+Q154R; and V82S+Q154R; N72K_V82S+Y123H+Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein or complex. This can alter the numbering of positions. However, the skilled person will understand that such corresponding mutations refer to the same mutation, e.g., Y73S and Y72S and D139M and D138M.

In some embodiments, the TadA*9 variant comprises the alterations described in Table 15 as described herein. In some embodiments, the TadA*9 variant is a monomer. In some embodiments, the TadA*9 variant is a heterodimer with a wild-type TadA adenosine deaminase. In some embodiments, the TadA*9 variant is a heterodimer with another TadA variant (e.g., TadA*8, TadA*9). Additional details of TadA*9 adenosine deaminases are described in International PCT Application No. PCT/US2020/049975, which is incorporated herein by reference for its entirety.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids. Typically, a cytidine deaminase catalyzes a C nucleobase that is positioned in the context of a single-stranded portion of a polynucleotide. In some embodiments, the entire polynucleotide comprising a target C can be single-stranded. For example, a cytidine deaminase incorporated into the base editor can deaminate a target C in a single-stranded RNA polynucleotide. In other embodiments, a base editor comprising a cytidine deaminase domain can act on a double-stranded polynucleotide, but the target C can be positioned in a portion of the polynucleotide which at the time of the deamination reaction is in a single-stranded state. For example, in embodiments where the NAGPB domain comprises a Cas9 domain, several nucleotides can be left unpaired during formation of the Cas9-gRNA-target DNA complex, resulting in formation of a Cas9 “R-loop complex”. These unpaired nucleotides can form a bubble of single-stranded DNA that can serve as a substrate for a single-strand specific nucleotide deaminase enzyme (e.g., cytidine deaminase).

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a base editor comprises all or a portion (e.g., a functional portion) of an APOBEC1 deaminase. In some embodiments, a deaminase incorporated into a base editor comprises all or a portion (e.g., a functional portion) of APOBEC2 deaminase. In some embodiments, a deaminase incorporated into a base editor comprises all or a portion (e.g., a functional portion) of is an APOBEC3 deaminase. In some embodiments, a deaminase incorporated into a base editor comprises all or a portion (e.g., a functional portion) of an APOBEC3A deaminase. In some embodiments, a deaminase incorporated into a base editor comprises all or a portion (e.g., a functional portion) of APOBEC3B deaminase. In some embodiments, a deaminase incorporated into a base editor comprises all or a portion (e.g., a functional portion) of APOBEC3C deaminase. In some embodiments, a deaminase incorporated into a base editor comprises all or a portion (e.g., a functional portion) of APOBEC3D deaminase. In some embodiments, a deaminase incorporated into a base editor comprises all or a portion (e.g., a functional portion) of APOBEC3E deaminase. In some embodiments, a deaminase incorporated into a base editor comprises all or a portion (e.g., a functional portion) of APOBEC3F deaminase. In some embodiments, a deaminase incorporated into a base editor comprises all or a portion (e.g., a functional portion) of APOBEC3G deaminase. In some embodiments, a deaminase incorporated into a base editor comprises all or a portion (e.g., a functional portion) of APOBEC3H deaminase. In some embodiments, a deaminase incorporated into a base editor comprises all or a portion (e.g., a functional portion) of APOBEC4 deaminase. In some embodiments, a deaminase incorporated into a base editor comprises all or a portion (e.g., a functional portion) of activation-induced deaminase (AID). In some embodiments a deaminase incorporated into a base editor comprises all or a portion (e.g., a functional portion) of cytidine deaminase 1 (CDA1). It should be appreciated that a base editor can comprise a deaminase from any suitable organism (e.g., a human or a rat). In some embodiments, a deaminase domain of a base editor is from a human, chimpanzee, gorilla, monkey, cow, dog, rat, or mouse. In some embodiments, the deaminase domain of the base editor is derived from rat (e.g., rat APOBEC1). In some embodiments, the deaminase domain of the base editor is human APOBEC1. In some embodiments, the deaminase domain of the base editor is pmCDA1.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins or complexes (e.g., base editors). For example, mutations that reduce, but do not eliminate, the catalytic activity of a deaminase domain within a base editing fusion protein or complex can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

For example, in some embodiments, an APOBEC deaminase incorporated into a base editor comprises one or more mutations selected from the group consisting of H121X, H122X, R126X, R126X, RI 18X, W90X, W90X, and R132X of rAPOBEC1, or one or more corresponding mutations in another APOBEC deaminase, wherein X is any amino acid. In some embodiments, an APOBEC deaminase incorporated into a base editor comprises one or more mutations selected from the group consisting of H121R, H122R, R126A, R126E, RI 18A, W90A, W90Y, and R132E of rAPOBEC1, or one or more corresponding mutations in another APOBEC deaminase.

In some embodiments, an APOBEC deaminase incorporated into a base editor comprises one or more mutations selected from the group consisting of D316X, D317X, R320X, R320X, R313X, W285X, W285X, R326X of hAPOBEC3G, or one or more corresponding mutations in another APOBEC deaminase, wherein X is any amino acid. In some embodiments, any of the fusion proteins or complexes provided herein comprise an APOBEC deaminase comprising one or more mutations selected from the group consisting of D316R, D317R, R320A, R320E, R313A, W285A, W285Y, R326E of hAPOBEC3G, or one or more corresponding mutations in another APOBEC deaminase.

In some embodiments, an APOBEC deaminase incorporated into a base editor comprises a H121R and a H122R mutation of rAPOBEC1, or one or more corresponding mutations in another APOBEC deaminase. In some embodiments an APOBEC deaminase incorporated into a base editor comprises an APOBEC deaminase comprising a R126A mutation of rAPOBEC1, or one or more corresponding mutations in another APOBEC deaminase. In some embodiments, an APOBEC deaminase incorporated into a base editor comprises an APOBEC deaminase comprising a R126E mutation of rAPOBEC1, or one or more corresponding mutations in another APOBEC deaminase. In some embodiments, an APOBEC deaminase incorporated into a base editor comprises an APOBEC deaminase comprising a R118A mutation of rAPOBEC1, or one or more corresponding mutations in another APOBEC deaminase. In some embodiments, an APOBEC deaminase incorporated into a base editor comprises an APOBEC deaminase comprising a W90A mutation of rAPOBEC1, or one or more corresponding mutations in another APOBEC deaminase. In some embodiments, an APOBEC deaminase incorporated into a base editor comprises an APOBEC deaminase comprising a W90Y mutation of rAPOBEC1, or one or more corresponding mutations in another APOBEC deaminase. In some embodiments, an APOBEC deaminase incorporated into a base editor comprises an APOBEC deaminase comprising a R132E mutation of rAPOBEC1, or one or more corresponding mutations in another APOBEC deaminase. In some embodiments an APOBEC deaminase incorporated into a base editor comprises an APOBEC deaminase comprising a W90Y and a R126E mutation of rAPOBEC1, or one or more corresponding mutations in another APOBEC deaminase. In some embodiments, an APOBEC deaminase incorporated into a base editor comprises an APOBEC deaminase comprising a R126E and a R132E mutation of rAPOBEC1, or one or more corresponding mutations in another APOBEC deaminase. In some embodiments, an APOBEC deaminase incorporated into a base editor comprises an APOBEC deaminase comprising a W90Y and a R132E mutation of rAPOBEC1, or one or more corresponding mutations in another APOBEC deaminase. In some embodiments, an APOBEC deaminase incorporated into a base editor comprises an APOBEC deaminase comprising a W90Y, R126E, and R132E mutation of rAPOBEC1, or one or more corresponding mutations in another APOBEC deaminase.

In some embodiments, an APOBEC deaminase incorporated into a base editor comprises an APOBEC deaminase comprising a D316R and a D317R mutation of hAPOBEC3G, or one or more corresponding mutations in another APOBEC deaminase. In some embodiments, any of the fusion proteins or complexes provided herein comprise an APOBEC deaminase comprising a R320A mutation of hAPOBEC3G, or one or more corresponding mutations in another APOBEC deaminase. In some embodiments, an APOBEC deaminase incorporated into a base editor comprises an APOBEC deaminase comprising a R320E mutation of hAPOBEC3G, or one or more corresponding mutations in another APOBEC deaminase. In some embodiments, an APOBEC deaminase incorporated into a base editor comprises an APOBEC deaminase comprising a R313A mutation of hAPOBEC3G, or one or more corresponding mutations in another APOBEC deaminase. In some embodiments, an APOBEC deaminase incorporated into a base editor comprises an APOBEC deaminase comprising a W285A mutation of hAPOBEC3G, or one or more corresponding mutations in another APOBEC deaminase. In some embodiments, an APOBEC deaminase incorporated into a base editor comprises an APOBEC deaminase comprising a W285Y mutation of hAPOBEC3G, or one or more corresponding mutations in another APOBEC deaminase. In some embodiments, an APOBEC deaminase incorporated into a base editor comprises an APOBEC deaminase comprising a R326E mutation of hAPOBEC3G, or one or more corresponding mutations in another APOBEC deaminase. In some embodiments, an APOBEC deaminase incorporated into a base editor comprises an APOBEC deaminase comprising a W285Y and a R320E mutation of hAPOBEC3G, or one or more corresponding mutations in another APOBEC deaminase. In some embodiments, an APOBEC deaminase incorporated into a base editor comprises an APOBEC deaminase comprising a R320E and a R326E mutation of hAPOBEC3G, or one or more corresponding mutations in another APOBEC deaminase. In some embodiments, an APOBEC deaminase incorporated into a base editor comprises an APOBEC deaminase comprising a W285Y and a R326E mutation of hAPOBEC3G, or one or more corresponding mutations in another APOBEC deaminase. In some embodiments, an APOBEC deaminase incorporated into a base editor comprises an APOBEC deaminase comprising a W285Y, R320E, and R326E mutation of hAPOBEC3G, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the present disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

The disclosure provides any deaminase domains with a certain percent identity plus any of the mutations or combinations thereof described herein. In some embodiments, the cytidine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the cytidine deaminases provided herein. In some embodiments, the cytidine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In embodiments, a fusion protein of the invention comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs).” In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, the adenosine deaminase variants of the invention comprise one or more alterations. In some embodiments, an adenosine deaminase variant of the invention is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some embodiments, the adenosine deaminase variant is a bacterial TadA deaminase variant (e.g., ecTadA). In some embodiments, the adenosine deaminase variant is a truncated TadA deaminase variant. In some embodiments, the adenosine deaminase variant is a fragment of a TadA deaminase variant. In some embodiments, an adenosine deaminase variant is a TadA*8 variant comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity of at least about 30%, 40%, 50% or more of the adenosine deaminase activity of a reference adenosine deaminase (e.g., TadA*8.20 or TadA*8.19). In some embodiments, an adenosine deaminase variant is a TadA*8.20 adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity of at least 30%, 40%, 50% of the activity of a reference adenosine deaminase (e.g., TadA*8.20 or TadA*8.19). In some embodiments, an adenosine deaminase variant as provided herein has an increased cytosine deaminase activity of at least about 10-fold, 20-fold, 30-fold, 40-fold, 50-fold, 60-fold, 70-fold, 80-fold, 90-fold, 100-fold or more relative to a reference adenosine deaminase (e.g., TadA*8.20 or TadA*8.19). In some embodiments, an adenosine deaminase variant as provided herein maintains a level of adenosine deaminase activity that is at least about 30%, 40%, 50%, 60%, 70% of the activity of a reference adenosine deaminase (e.g., TadA*8.20 or TadA*8.19). In some embodiments, the reference adenosine deaminase is TadA*8.20 or TadA*8.19.

In some embodiments, the adenosine deaminase variant is an adenosine deaminase comprising one or more alterations that increase cytosine deaminase activity and has an amino acid sequence that is at least about 70%, 75%, 80%, 85%, 90%, 95%, 98%, 99% or greater identity to SEQ ID NO: 1 below:

(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD

In some embodiments, the adenosine deaminase variant is an adenosine deaminase comprising the amino acid sequence of SEQ ID NO: 1 and one or more alterations that increase cytosine deaminase activity. In various embodiments, the one or more alterations of the invention do not include a R amino acid at position 48 of SEQ ID NO: 1, or a corresponding mutation in another adenosine deaminase.

In some embodiments, the adenosine deaminase variant is an adenosine deaminase comprising one or more alterations at an amino acid position selected from 2, 4, 6, 13, 27, 29, 100, 112, 114, 115, 162, and 165 of an amino acid sequence having at least about 70%, 75%, 80%, 85%, 90%, 95%, 98%, 99% or greater identity to SEQ ID NO: 1, or a corresponding alteration in another deaminase. In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. In some embodiments, the two or more alterations are at an amino acid position selected from the group consisting of S2X, V4X, F6X, H8X, R13X, T17X, R23X, E27X, P29X, V30X, R47X, A48X, I49X, G67X, Y76X, D77X, S82X, F84X, H96X, G100X, R107X, G112X, A114X, G115X, M118X, D119X, H122X, N127X, A142X, A143X, R147X, Y147X, F149X, A158X, Q159X, A162X, S165X, T166X, and D167X of an amino acid sequence having at least about 70%, 75%, 80%, 85%, 90%, 95%, 98%, 99% or greater identity to SEQ ID NO: 1, or a corresponding alteration in another deaminase. In various embodiments, the alterations of the invention do not include a 48R mutation. In some embodiments, the adenosine deaminase variant is an adenosine deaminase comprising one or more alterations at an amino acid position selected from 2, 4, 6, 13, 27, 29, 100, 112, 114, 115, 162, and 165 of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, I49M, I49N, I49Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N1271, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising a combination of amino acid alterations selected from: E27H, Y76I, and F84M; E27H, I49K, and Y76I; E27S, I49K, Y76I, and A162N; E27K and D119N; E27H and Y76I; E27S, I49K, and G67W; E27S, 149K, and Y761; 149T, G67W, and H96N; E27C, Y76I, and D119N; R13G, E27Q, and N127K; T17A, E27H, 149M, Y761, and M118L; I49Q, Y76I, and G115M; S2H, I49K, Y76I, and G112H; R47S and R107C; H8Q, I49Q, and Y76I; T17A, A48G, S82T, and A142E; E27G and I49N; E27G, D77G, and S165P; E27S, I49K, and S82T; E27S, I49K, S82T, and Gi 15M; E27S, V301, 149K, and S82T; E27S, V30F, I49K, S82T, F84A, R107C, and A142E; E27S, V30F, I49K, S82T, F84A, G112H, and A142E; E27S, V30F, I49K, S82T, F84A, Gi 15M, and A142E; E27S, I49K, S82T, F84L, and R107C; E27S, I49K, S82T, F84L, and G112H; E27S, I49K, S82T, F84L, and G115M; E27S, I49K, S82T, F84L, R107C, and Gi 12H; E27S, I49K, S82T, F84L, R107C, and Gi 15M; E27S, I49K, S82T, F84L, R107C, and A142E; E27S, I49K, S82T, F84L, Gi 12H, and A142E; E27S, I49K, S82T, F84L, G115M, and A142E; E27S, I49K, S82T, F84L, R107C, G112H, G115M, and A142E; E27S, V301, 149K, S82T, and F84L; E27S, P29G, I49K, and S82T; E27S, P29G, I49K, S82T, and G115M; E27S, P29G, I49K, S82T, and A142E; P29G, I49K, and S82T; E27G, I49K, and S82T; E27G, I49K, S82T, R107C, and A142E; V4K, E27H, I49K, Y76I, and A114C; V4K, E27H, I49K, Y76I, and D77G; F6Y, E27H, I49K, Y76I, G100A, and H122R; V4T, E27H, I49K, Y76R, and H122G; F6Y, E27H, I49K, and Y76W; F6Y, E27H, I49K, Y76I, and D119N; F6Y, E27H, I49K, Y76I, and A114C; F6Y, E27H, I49K, and Y76I; V4K, E27H, 149K, Y76W, and H122T; F6G, E27H, I49K, Y76R, and G100K; F6H, E27H, I49K, Y76I, and H122N; E27H, I49K, Y76I, and A114C; F6Y, E27H, I49K, Y76H, H122R, and T1661; E27H, 149K, Y761, and N127P; R23Q, E27H, I49K, and Y76R; E27H, I49K, Y76H, H122R, and A158V; F6Y, E27H, I49K, Y76I, and T111H; E27H, I49K, Y76I, and R147H; E27H, 149K, Y761, and A143E; F6Y, E27H, I49K, and Y76R; T17W, E27H, I49K, Y76H, H122G, and A158V; V4S, E27H, I49K, A143E, and Q159S; E27H, I49K, Y76I, N127I, and A162Q; T17A, E27H, and A48G; T17A, E27K, and A48G; T17A, E27S, and A48G; T17A, E27S, A48G, and I49K; T17A, E27G, and A48G; T17A, A48G, and I49N; T17A, E27G, A48G, and I49N; T17A, E27Q, and A48G; E27S, I49K, S82T, and R107C; E27S, I49K, S82T, and Gi 12H; E27S, I49K, S82T, and A142E; E27S, I49K, S82T, R107C, and G112H; E27S, I49K, S82T, R107C, and G115M; E27S, 149K, S82T, R107C, and A142E; E27S, I49K, S82T, G112H, and A142E; E27S, 149K, S82T, G115M, and A142E; E27S, I49K, S82T, R107C, G112H, G115M, and A142E; E27S, V301, 149K, S82T, and R107C; E27S, V301, 149K, S82T, and G112H; E27S, V301, 149K, S82T, and G115M; E27S, V301, 149K, S82T, and A142E; E27S, V301, 149K, S82T, R107C, and G112H; E27S, V301, I49K, S82T, R107C, and Gi 15M; E27S, V301, 149K, S82T, R107C, and A142E; E27S, V301, I49K, S82T, G112H, and A142E; E27S, V301, 149K, S82T, G115M, and A142E; E27S, V30I, I49K, S82T, R107C, Gi 12H, Gi 15M, and A142E; E27S, V30L, I49K, and S82T; E27S, V30L, I49K, S82T, and R107C; E27S, V30L, I49K, S82T, and Gi 12H; E27S, V30L, 149K, S82T, and G115M; E27S, V30L, I49K, S82T, and A142E; E27S, V30L, I49K, S82T, R107C, and G112H; E27S, V30L, I49K, S82T, R107C, and Gi 15M; E27S, V30L, I49K, S82T, R107C, and A142E; E27S, V30L, I49K, S82T, Gi 12H, and A142E; E27S, V30L, I49K, S82T, Gi 15M, and A142E; E27S, V30L, I49K, S82T, R107C, Gi 12H, Gi 15M, and A142E; E27S, V30F, 149K, S82T, and F84A; E27S, V30F, I49K, S82T, F84A, and R107C; E27S, V30F, I49K, S82T, F84A, and G112H; E27S, V30F, I49K, S82T, F84A, and G115M; E27S, V30F, I49K, S82T, F84A, and A142E; E27S, V30F, I49K, S82T, F84A, R107C, and G112H; E27S, V30F, I49K, S82T, F84A, R107C, and G115M; E27S, V30F, I49K, S82T, F84A, R107C, G112H, G115M, and A142E; E27S, I49K, S82T, and F84L; E27S, I49K, S82T, F84L, and A142E; E27S, V301, 149K, S82T, F84L, and R107C; E27S, V301, 149K, S82T, F84L, and G112H; E27S, V301, 149K, S82T, F84L, and Gi 15M; E27S, V301, 149K, S82T, F84L, and A142E; E27S, V301, 149K, S82T, F84L, R107C, and G112H; E27S, V301, 149K, S82T, F84L, R107C, and G115M; E27S, V301, 149K, S82T, F84L, R107C, and A142E; E27S, V301, 149K, S82T, F84L, Gi 12H, and A142E; E27S, V301, 149K, S82T, F84L, Gi 15M, and A142E; E27S, V301, 149K, S82T, F84L, R107C, Gi 12H, G115M, and A142E; E27S, P29G, I49K, S82T, and R107C; E27S, P29G, 149K, S82T, and G112H; E27S, P29G, I49K, S82T, R107C, and G112H; E27S, P29G, I49K, S82T, R107C, and G115M; E27S, P29G, I49K, S82T, R107C, and A142E; E27S, P29G, 149K, S82T, G112H, and A142E; E27S, P29G, I49K, S82T, G115M, and A142E; E27S, P29G, I49K, S82T, R107C, G112H, G115M, and A142E; P29G, I49K, S82T, and R107C; P29G, 149K, S82T, and G112H; P29G, I49K, S82T, and G115M; P29G, I49K, S82T, and A142E; P29G, I49K, S82T, R107C, and G112H; P29G, I49K, S82T, R107C, and G115M; P29G, I49K, S82T, R107C, and A142E; P29G, I49K, S82T, G112H, and A142E; P29G, I49K, S82T, G115M, and A142E; P29G, I49K, S82T, R107C, G112H, G115M, and A142E; P29K, I49K, and S82T; P29K, 149K, S82T, and R107C; P29K, I49K, S82T, and G112H; P29K, I49K, S82T, and G115M; P29K, I49K, S82T, and A142E; P29K, I49K, S82T, R107C, and G112H; P29K, I49K, S82T, R107C, and G115M; P29K, I49K, S82T, R107C, and A142E; P29K, I49K, S82T, Gi 12H, and A142E; P29K, I49K, S82T, Gi 15M, and A142E; P29K, I49K, S82T, R107C, Gi 12H, Gi 15M, and A142E; P29K, V301, 149K, and S82T; P29K, V301, 149K, S82T, and R107C; P29K, V301, 149K, S82T, and G112H; P29K, V301, 149K, S82T, and Gi 15M; P29K, V301, 149K, S82T, and A142E; P29K, V301, 149K, S82T, R107C, and G112H; P29K, V301, 149K, S82T, R107C, and G115M; P29K, V301, 149K, S82T, R107C, and A142E; P29K, V301, 149K, S82T, Gi 12H, and A142E; P29K, V301, 149K, S82T, G115M, and A142E; P29K, V301, 149K, S82T, R107C, G112H, G115M, and A142E; P29K, I49K, S82T, and F84L; P29K, I49K, S82T, F84L, and R107C; P29K, I49K, S82T, F84L, and G112H; P29K, I49K, S82T, F84L, and G115M; P29K, I49K, S82T, F84L, and A142E; P29K, I49K, S82T, F84L, R107C, and G112H; P29K, I49K, S82T, F84L, R107C, and G115M; P29K, I49K, S82T, F84L, R107C, and A142E; P29K, I49K, S82T, F84L, G112H, and A142E; P29K, I49K, S82T, F84L, G115M, and A142E; P29K, I49K, S82T, F84L, R107C, G112H, G115M, and A142E; P29K, V301, 149K, S82T, and F84L; P29K, V301, 149K, S82T, F84L, and R107C; P29K, V301, 149K, S82T, F84L, and Gi 12H; P29K, V301, 149K, S82T, F84L, and G115M; P29K, V301, 149K, S82T, F84L, and A142E; P29K, V301, 149K, S82T, F84L, R107C, and G112H; P29K, V301, 149K, S82T, F84L, R107C, and G115M; P29K, V301, I49K, S82T, F84L, R107C, and A142E; P29K, V301, 149K, S82T, F84L, G112H, and A142E; P29K, V301, 149K, S82T, F84L, G115M, and A142E; P29K, V301, 149K, S82T, F84L, R107C, G112H, G115M, and A142E; E27G, I49K, S82T, and R107C; E27G, 149K, S82T, and G112H; E27G, I49K, S82T, and Gi 15M; E27G, I49K, S82T, and A142E; E27G, I49K, S82T, R107C, and Gi 12H; E27G, I49K, S82T, R107C, and Gi 15M; E27G, I49K, S82T, Gi 12H, and A142E; E27G, I49K, S82T, G115M, and A142E; E27G, I49K, S82T, R107C, G112H, G115M, and A142E; E27H, I49K, and S82T; E27H, I49K, S82T, and R107C; E27H, 149K, S82T, and G112H; E27H, I49K, S82T, and G115M; E27H, I49K, S82T, and A142E; E27H, I49K, S82T, R107C, and G112H; E27H, I49K, S82T, R107C, and G115M; E27H, I49K, S82T, R107C, and A142E; E27H, I49K, S82T, G112H, and A142E; E27H, I49K, S82T, G115M, and A142E; E27H, I49K, S82T, R107C, G112H, G115M, and A142E; E27S, and S82T; E27S, S82T, and R107C; E27S, S82T, and Gi 12H; E27S, S82T, and Gi 15M; E27S, S82T, and A142E; E27S, S82T, R107C, and G112H; E27S, S82T, R107C, and G115M; E27S, S82T, R107C, and A142E; E27S, S82T, G112H, and A142E; E27S, S82T, G115M, and A142E; E27S, S82T, R107C, G112H, G115M, and A142E; P29A, and S82T; P29A, S82T, and R107C; P29A, S82T, and G112H; P29A, S82T, and G115M; P29A, S82T, and A142E; P29A, S82T, R107C, and G112H; P29A, S82T, R107C, and G115M; P29A, S82T, R107C, and A142E; P29A, S82T, G112H, and A142E; P29A, S82T, G115M, and A142E; P29A, S82T, R107C, G112H, G115M, and A142E; E27S, V30I, and S82T; E27S, V30I, S82T, and R107C; E27S, V30I, S82T, and G112H; E27S, V30I, S82T, and Gi 15M; E27S, V30I, S82T, and A142E; E27S, V301, S82T, R107C, and G112H; E27S, V30I, S82T, R107C, and G115M; E27S, V30I, S82T, R107C, and A142E; E27S, V30I, S82T, Gi 12H, and A142E; E27S, V30I, S82T, Gi 15M, and A142E; E27S, V30I, S82T, R107C, G112H, G115M, and A142E; P29A, V30I, S82T, and F84L; P29A, V30I, S82T, F84L, and R107C; P29A, V30I, S82T, F84L, and Gi 12H; P29A, V30I, S82T, F84L, and Gi 15M; P29A, V30I, S82T, F84L, and A142E; P29A, V30I, S82T, F84L, R107C, and Gi 12H; P29A, V30I, S82T, F84L, R107C, and Gi 15M; P29A, V30I, S82T, F84L, R107C, and A142E; P29A, V30I, S82T, F84L, G112H, and A142E; P29A, V30I, S82T, F84L, G115M, and A142E; P29A, V30I, S82T, F84L, R107C, G112H, G115M, and A142E; E27S, P29A, V30L, I49K, S82T, F84L, R107C, G112H, G115M, and A142E; V4K, and A114C; V4K, and D77G; F6Y, G100A, and H122R; V4T, I76R, and H122G; F6Y, and I76W; F6Y, and D119N; F6Y, and A114C; V4K, I76W, and H122T; F6G, I76R, and G100K; F6H, and H122N; F6Y, 176H, H122R, and T1661; R23Q, and I76R; I76H, H122R, and A158V; F6Y, and T111H; T111H, H122G, and A162C; F6Y, and I76R; T17W, I76H, H122G, and A158V; V4S, I76Y, A143E, and Q159S; N127I, A162Q; E27H, Y76I, F84M, and F149Y; E27H, I49K, Y76I, and F149Y; T17A, E27H, 149M, Y76I, M118L, and F149Y; T17A, A48G, S82T, A142E, and F149Y; E27G, and F149Y; E27G, I49N, and F149Y; E27H, Y76I, F84M, Y147D, F149Y, T166I, and D167N; E27H, I49K, Y76I, Y147D, F149Y, T166I, D167N; T17A, E27H, I49M, Y76I, M118L, Y147D, F149Y, T1661, and D167N; T17A, A48G, S82T, A142E, Y147D, F149Y, T166I, and D167N; E27G, Y147D, F149Y, T166I, and D167N; E27G, I49N, Y147D, F149Y, T166I, and D167N; F6Y, E27H, I49K, Y76W, S82T, R107C, G112H, G115M, and A142E; F6Y, E27H, I49K, D77G, S82T, R107C, G112H, G115M, and A142E; F6Y, E27H, I49K, S82T, R107C, G112H, A114C, G115M, and A142E; F6Y, E27H, I49K, S82T, R107C, G112H, G115M, D119N, and A142E; F6Y, E27H, I49K, S82T, R107C, G112H, G115M, H122G, and A142E; F6Y, E27H, I49K, S82T, R107C, G112H, G115M, N127P, and A142E; F6Y, E27H, I49K, S82T, R107C, G112H, G115M, A142E, and A143E; F6Y, E27H, I49K, S82T, R107C, G112H, G115M, and A143E; F6Y, E27H, I49K, Y76W, D77G, S82T, R107C, G112H, G115M, and A142E; F6Y, E27H, I49K, Y76W, S82T, R107C, G112H, A114C, G115M, and A142E; F6Y, E27H, I49K, Y76W, S82T, R107C, G112H, G115M, D119N, and A142E; F6Y, E27H, I49K, Y76W, S82T, R107C, G112H, G115M, H122G, and A142E; F6Y, E27H, I49K, Y76W, S82T, R107C, G112H, G115M, N127P, and A142E; F6Y, E27H, I49K, Y76W, S82T, R107C, G112H, G115M, A142E, and A143E; F6Y, E27H, I49K, Y76W, S82T, R107C, Gi 12H, Gi 15M, and A143E; F6Y, E27H, I49K, D77G, S82T, R107C, G112H, A114C, G115M, and A142E; F6Y, E27H, I49K, D77G, S82T, R107C, G112H, G115M, D119N, and A142E; F6Y, E27H, I49K, D77G, S82T, R107C, G112H, G115M, H122G, and A142E; F6Y, E27H, I49K, D77G, S82T, R107C, G112H, G115M, N127P, and A142E; F6Y, E27H, I49K, D77G, S82T, R107C, G112H, G115M, A142E, and A143E; F6Y, E27H, I49K, D77G, S82T, R107C, Gi 12H, Gi 15M, and A143E; F6Y, E27H, I49K, S82T, R107C, G112H, A114C, G115M, D119N, and A142E; F6Y, E27H, I49K, S82T, R107C, Gi 12H, A114C, G115M, H122G, and A142E; F6Y, E27H, I49K, S82T, R107C, G112H, A114C, G115M, N127P, and A142E; F6Y, E27H, I49K, S82T, R107C, G112H, G115M, D119N, H122G, and A142E; F6Y, E27H, I49K, S82T, R107C, G112H, G115M, D119N, N127P, and A142E; F6Y, E27H, I49K, S82T, R107C, G112H, G115M, H122G, N127P, and A142E; F6Y, E27H, I49K, Y76W, D77G, S82T, R107C, G112H, A114C, G115M, and A142E; F6Y, E27H, I49K, Y76W, D77G, S82T, R107C, G112H, Gi 15M, D119N, and A142E; F6Y, E27H, I49K, Y76W, D77G, S82T, R107C, G112H, G115M, H122G, and A142E; F6Y, E27H, I49K, Y76W, D77G, S82T, R107C, G112H, Gi 15M, N127P, and A142E; F6Y, E27H, I49K, Y76W, D77G, S82T, R107C, G112H, G115M, A142E, and A143E; F6Y, E27H, I49K, Y76W, D77G, S82T, R107C, Gi 12H, Gi 15M, and A143E; F6Y, E27H, I49K, Y76W, S82T, R107C, G112H, A114C, G115M, D119N, and A142E; F6Y, E27H, I49K, Y76W, S82T, R107C, G112H, A114C, G115M, H122G, and A142E; F6Y, E27H, I49K, Y76W, S82T, R107C, G112H, A114C, G115M, N127P, and A142E; F6Y, E27H, I49K, Y76W, S82T, R107C, G112H, A114C, G115M, A142E, and A143E; F6Y, E27H, I49K, Y76W, S82T, R107C, G112H, A114C, G115M, and A143E; F6Y, E27H, I49K, Y76W, S82T, R107C, G112H, G115M, D119N, H122G, and A142E; F6Y, E27H, I49K, Y76W, S82T, R107C, G112H, G115M, D119N, N127P, and A142E; F6Y, E27H, I49K, Y76W, S82T, R107C, Gi 12H, Gi 15M, H122G, N127P, and A142E; F6Y, E27H, I49K, Y76W, S82T, R107C, G112H, A114C, G115M, D119N, H122G, and A142E; F6Y, E27H, I49K, Y76W, S82T, R107C, G112H, A114C, G115M, D119N, N127P, and A142E; F6Y, E27H, I49K, Y76W, S82T, R107C, G112H, A114C, G115M, H122G, N127P, and A142E; F6Y, E27H, I49K, Y76W, D77G, S82T, R107C, G112H, A114C, G115M, D119N, and A142E; F6Y, E27H, I49K, Y76W, D77G, S82T, R107C, Gi 12H, A114C, Gi 15M, H122G, and A142E; F6Y, E27H, I49K, Y76W, D77G, S82T, R107C, Gi 12H, A114C, Gi 15M, N127P, and A142E; F6Y, E27H, I49K, Y76W, D77G, S82T, R107C, Gi 12H, A114C, G115M, A142E, and A143E; F6Y, E27H, I49K, Y76W, D77G, S82T, R107C, G112H, A114C, G115M, and A143E; F6Y, E27H, I49K, Y76W, D77G, S82T, R107C, G112H, A114C, G115M, D119N, H122G, and A142E; F6Y, E27H, I49K, Y76W, D77G, S82T, R107C, G112H, A114C, G115M, D119N, N127P, and A142E; F6Y, E27H, I49K, Y76W, D77G, S82T, R107C, Gi 12H, A114C, Gi 15M, H122G, N127P, and A142E; F6Y, E27H, I49K, Y76W, S82T, R107C, G112H, A114C, G115M, D119N, H122G, N127P, A142E, and A143E; F6Y, E27H, I49K, Y76W, S82T, R107C, G112H, A114C, G115M, D119N, H122G, N127P, and A143E; F6Y, E27H, I49K, Y76W, D77G, S82T, R107C, G112H, A114C, G115M, D119N, H122G, N127P, A142E, and A143E; and F6Y, E27H, I49K, Y76W, D77G, S82T, R107C, G112H, A114C, G115M, D119N, H122G, N127P, and A143E; of an amino acid sequence having at least about 70%, 75%, 80%, 85%, 90%, 95%, 98%, 99% or greater identity to SEQ ID NO: 1, or a corresponding combination of alterations in another deaminase.

In some embodiments, the adenosine deaminase variant is an adenosine deaminase comprising an amino acid alteration or combination of amino acid alterations selected from those listed in any of Tables 5.2A-5.2F.

The residue identity of exemplary adenosine deaminase variants that are capable of deaminating adenine and/or cytidine in a target polynucleotide (e.g., DNA) is provided in Tables 5.2A-5.2F below. Further examples of adenosine deaminse variants include the following variants of 1.17 (see Table 5.2A): 1.17+E27H; 1.17+E27K; 1.17+E27S; 1.17+E27S+I49K; 1.17+E27G; 1.17+I49N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, base editing is carried out to induce therapeutic changes in the genome of a cell of a subject (e.g., human). Cells are collected from a subject and contacted with one or more guide RNAs and a nucleobase editor polypeptide comprising a nucleic acid programmable DNA binding protein (napDNAbp) (e.g., Cas9) and an adenosine deaminase variant capable of deaminating both adenine and cytosine in a target polynucleotide (e.g., DNA).

In some embodiments, cells are contacted with one or more guide RNAs and a fusion protein comprising a nucleic acid programmable DNA binding protein (napDNAbp) (e.g., Cas9) and an adenosine deaminase variant capable of deaminating both adenine and cytosine in a target polynucleotide (e.g., DNA). In some embodiments, the napDNAbp is a Cas9.

In some embodiments, cells are contacted with a multi-molecular complex. In some embodiments, cells are contacted with a base editor system as provided herein. In some embodiments, the base editor systems as provided herein comprise an adenosine base editor (ABE) variant (e.g., a CABE). In some embodiments, the CABE variant is an ABE8 variant. In some embodiments, the ABE8 variant is an ABE8.20 variant. In some embodiments, CABEs as provided herein have both A to G and C to T base editing activity. Therefore, multiple edits may be introduced into the genome of a subject (e.g., human). The ability to target both A to G and C to T base editing activity allows for diverse targeting of polynucleotides in the genome in a subject to treat a genetic disease or disorder.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 5.2A
Adenosine Deaminase Variants (CABE-1; T_{AD}AC-1). Mutations are indicated with reference to TadA*8.20.

| | location in structure | | | | | | | | | | | | | |
|---|---|---|---|---|---|---|---|---|---|---|---|---|---|---|
| | | | | | near | near | near | near | | | | | | |
| | | surface | surface | surface | active | active | active | active | | | | | | |
| | N/A | h1 | h1 | h1 | site | site | site | site | | surface | | | | |
| | Amino Acid No. (*START Met is AA#1) | | | | | | | | | | | | | |
| | 2 | 8 | 13 | 17 | 27 | 47 | 48 | 49 | 67 | 76 | 77 | | | |
| | TadA*8.20 | | | | | | | | | | | | | |
| | S | H | R | T | E | R | A | I | G | Y | D | | | |
| | TadA*8.19 | | | | | | | | | | | Plasmid #s | | |
| | | | | | | | | | | I | | without ugi | with ugi | bacterial |
| 1.1 | | | | | H | | | | | I | | pDKL-11 | pDKL-23 | pDKL-35 |
| 1.2 | | | | | H | | | K | | I | | pDKL-12 | pDKL-24 | pDKL-36 |
| 1.3 | | | | | S | | | K | | I | | pDKL-13 | pDKL-25 | pDKL-37 |
| 1.4 | | | | | S | | | K | | I | | pDKL-14 | pDKL-26 | pDKL-38 |
| 1.5 | | | | | K | | | | | | | pDKL-15 | pDKL-27 | pDKL-39 |
| 1.6 | | | | | K | | | | | | | pDKL-16 | pDKL-28 | pDKL-40 |
| 1.7 | | | | | H | | | | | I | | pDKL-17 | pDKL-29 | pDKL-41 |
| 1.8 | | | | | S | | | K | W | | | pDKL-18 | pDKL-30 | pDKL-42 |
| 1.9 | | | | | | | | T | W | | | pDKL-19 | pDKL-31 | pDKL-43 |
| 1.10 | | | | | C | | | | | I | | pDKL-20 | pDKL-32 | pDKL-44 |
| 1.11 | | | G | | Q | | | | | | | pDKL-21 | pDKL-33 | pDKL-45 |
| 1.12 | | | | A | H | | | M | | I | | pDKL-22 | pDKL-34 | pDKL-46 |
| 1.13 | | | | | | | | Q | | I | | pDKL-47 | pDKL-55 | pDKL-63 |
| 1.14 | H | | | | | | | K | | I | | pDKL-48 | pDKL-56 | pDKL-64 |
| 1.15 | | | | | | S | | | | | | pDKL-49 | pDKL-57 | pDKL-65 |
| 1.16 | | Q | | | | | | Q | | I | | pDKL-50 | pDKL-58 | pDKL-66 |
| 1.17 | | | | A | | | G | | | | | pDKL-51 | pDKL-59 | pDKL-67 |
| 1.18 | | | | | G | | | | | | | pDKL-52 | pDKL-60 | pDKL-68 |
| 1.19 | | | | | G | | | N | | | | pDKL-53 | pDKL-61 | pDKL-69 |
| 1.20 | | | | | G | | | | | | G | pDKL-54 | pDKL-62 | pDKL-70 |

| | location in structure | | | | | | | | | | | | | | |
|---|---|---|---|---|---|---|---|---|---|---|---|---|---|---|---|
| | | near | | near | | | | | | | | | | | |
| | | active | | active | | | | | | | | | | | |
| | internal | site | | site | surface | | surface | surface | surface | | surface | | | | |
| | Amino Acid No. (*START Met is AA#1) | | | | | | | | | | | | | | |
| | 82 | 84 | 96 | 107 | 112 | 115 | 118 | 119 | 127 | 142 | 162 | 165 | | | |
| | TadA*8.20 | | | | | | | | | | | | | | |
| | S | F | H | R | G | G | M | D | N | A | A | S | Plasmid #s | | |
| | TadA*8.19 | | | | | | | | | | | | without ugi | with ugi | bacterial |
| 1.1 | | M | | | | | | | | | | | pDKL-11 | pDKL-23 | pDKL-35 |
| 1.2 | | | | | | | | | | | | | pDKL-12 | pDKL-24 | pDKL-36 |
| 1.3 | | | | | | | | | | | | | pDKL-13 | pDKL-25 | pDKL-37 |
| 1.4 | | | | | | | | | | | N | | pDKL-14 | pDKL-26 | pDKL-38 |
| 1.5 | | | | | | | | | | | | | pDKL-15 | pDKL-27 | pDKL-39 |
| 1.6 | | | | | | | | N | | | | | pDKL-16 | pDKL-28 | pDKL-40 |
| 1.7 | | | | | | | | | | | | | pDKL-17 | pDKL-29 | pDKL-41 |
| 1.8 | | | | | | | | | | | | | pDKL-18 | pDKL-30 | pDKL-42 |
| 1.9 | | | N | | | | | | | | | | pDKL-19 | pDKL-31 | pDKL-43 |
| 1.10 | | | | | | | | N | | | | | pDKL-20 | pDKL-32 | pDKL-44 |
| 1.11 | | | | | | | | | K | | | | pDKL-21 | pDKL-33 | pDKL-45 |
| 1.12 | | | | | | | L | | | | | | pDKL-22 | pDKL-34 | pDKL-46 |
| 1.13 | | | | | | M | | | | | | | pDKL-47 | pDKL-55 | pDKL-63 |
| 1.14 | | | | | H | | | | | | | | pDKL-48 | pDKL-56 | pDKL-64 |
| 1.15 | | | | C | | | | | | | | | pDKL-49 | pDKL-57 | pDKL-65 |
| 1.16 | | | | | | | | | | | | | pDKL-50 | pDKL-58 | pDKL-66 |
| 1.17 | T | | | | | | | | | E | | | pDKL-51 | pDKL-59 | pDKL-67 |
| 1.18 | | | | | | | | | | | | | pDKL-52 | pDKL-60 | pDKL-68 |
| 1.19 | | | | | | | | | | | | | pDKL-53 | pDKL-61 | pDKL-69 |
| 1.20 | | | | | | | | | | | | P | pDKL-54 | pDKL-62 | pDKL-70 |

TABLE 5.2B
Rationally Designed Candidate Editors (CABE-2s; TADAC-
2s). Mutations are indicated with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| $1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| $1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| $1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| $1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | I | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | H | | C | H | | |
| S1.150 | H | | | K | T | | C | | M | |
| S1.151 | H | | | K | T | | | | | E |
| $1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| $1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 5.2C
Candidate base editors (CABE-2e; TADAC-2e). Mutations are
indicated with reference to variant 1.2 (Table 5.2A) .

| | Alternative Variant | Residue identity (START Met is amino acid #1) | | | | | | | | | |
|---|---|---|---|---|---|---|---|---|---|---|---|
| Variant Name | Names | 4 | 6 | 17 | 23 | 76 | 77 | 100 | 111 | 114 | 119 |
| Reference | 1.2 (see Table 1A) | V | F | T | R | I | D | G | T | A | D |
| TadAC2.1 | pDKL-135; 2.1 | K | | | | | | | | C | |
| TadAC2.2 | pDKL-136; 2.2 | K | | | | | G | | | | |
| TadAC2.3 | pDKL-137; 2.3 | | Y | | | | | A | | | |
| TadAC2.4 | pDKL-138; 2.4 | T | | | | R | | | | | |
| TadAC2.5 | pDKL-139; 2.5 | | Y | | | W | | | | | |
| TadAC2.6 | pDKL-140; 2.6 | | Y | | | | | | | | N |
| TadAC2.7 | pDKL-141; 2.7 | | Y | | | | | | | C | |
| TadAC2.8 | pDKL-142; 2.8 | | Y | | | | | | | | |
| TadAC2.9 | pDKL-143; 2.9 | K | | | | W | | | | | |
| TadAC2.10 | pDKL-144; 2.10 | | G | | | R | | K | | | |
| TadAC2.11 | pDKL-145; 2.11 | | H | | | | | | | | |
| TadAC2.12 | pDKL-146; 2.12 | | | | | | | | | C | |
| TadAC2.13 | pDKL-147; 2.13 | | Y | | | H | | | | | |
| TadAC2.14 | pDKL-148; 2.14 | | | | | | | | | | |
| TadAC2.15 | pDKL-149; 2.15 | | | | Q | R | | | | | |
| TadAC2.16 | pDKL-150; 2.16 | | | | | H | | | | | |
| TadAC2.17 | pDKL-151; 2.17 | | Y | | | | | | H | | |
| TadAC2.18 | pDKL-152; 2.18 | | | | | W | | | | | |
| TadAC2.19 | pDKL-153; 2.19 | | | | | | | | H | | |
| TadAC2.20 | pDKL-154; 2.20 | | | | | | | | | | |
| TadAC2.21 | pDKL-155; 2.21 | | Y | | | R | | | | | |
| TadAC2.22 | pDKL-156; 2.22 | | | W | | H | | | | | |
| TadAC2.23 | pDKL-157; 2.23 | S | | | | Y | | | | | |
| TadAC2.24 | pDKL-158; 2.24 | | | | | | | | | | |

| | Alternative Variant | Residue identity (START Met is amino acid #1) | | | | | | | |
|---|---|---|---|---|---|---|---|---|---|
| Variant Name | Names | 122 | 127 | 143 | 147 | 158 | 159 | 162 | 166 |
| Reference | 1.2 (see Table 1A) | H | N | A | R | A | Q | A | T |
| TadAC2.1 | pDKL-135; 2.1 | | | | | | | | |
| TadAC2.2 | pDKL-136; 2.2 | | | | | | | | |
| TadAC2.3 | pDKL-137; 2.3 | R | | | | | | | |
| TadAC2.4 | pDKL-138; 2.4 | G | | | | | | | |
| TadAC2.5 | pDKL-139; 2.5 | | | | | | | | |
| TadAC2.6 | pDKL-140; 2.6 | | | | | | | | |
| TadAC2.7 | pDKL-141; 2.7 | | | | | | | | |
| TadAC2.8 | pDKL-142; 2.8 | | | | | | | | |
| TadAC2.9 | pDKL-143; 2.9 | T | | | | | | | |
| TadAC2.10 | pDKL-144; 2.10 | | | | | | | | |
| TadAC2.11 | pDKL-145; 2.11 | N | | | | | | | |
| TadAC2.12 | pDKL-146; 2.12 | | | | | | | | |
| TadAC2.13 | pDKL-147; 2.13 | R | | | | | | | I |
| TadAC2.14 | pDKL-148; 2.14 | | P | | | | | | |
| TadAC2.15 | pDKL-149; 2.15 | | | | | | | | |
| TadAC2.16 | pDKL-150; 2.16 | R | | | | V | | | |
| TadAC2.17 | pDKL-151; 2.17 | | | | | | | | |
| TadAC2.18 | pDKL-152; 2.18 | | | | | | | | |
| TadAC2.19 | pDKL-153; 2.19 | G | | | | | | C | |
| TadAC2.20 | pDKL-154; 2.20 | | | E | | | | | |
| TadAC2.21 | pDKL-155; 2.21 | | | | | | | | |
| TadAC2.22 | pDKL-156; 2.22 | G | | | | V | | | |
| TadAC2.23 | pDKL-157; 2.23 | | | E | | | S | | |
| TadAC2.24 | pDKL-158; 2.24 | | I | | | | | Q | |

TABLE 5.2D
Rationally Designed Candidate Editors (CBE-T1; TADC-
1). Mutations are indicated with reference to ABE8.20m.

| | AA Positions | | | | | | | | | | | | | | |
|---|---|---|---|---|---|---|---|---|---|---|---|---|---|---|---|
| | 6 | 27 | 49 | 76 | 77 | 82 | 107 | 112 | 114 | 115 | 119 | 122 | 127 | 142 | 143 |
| | TadA8.20 | | | | | | | | | | | | | | |
| | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table 1C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | | | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | GT | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | G | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 5.2E
Hybrid constructs. Mutations are indicated with reference to ABE7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| ABE7.10 | I | V | A | T | D | H | Y | Y | F | Q | T | D |
| ABE8e | | | S | R | N | N | | D | Y | | I | N |
| ABE8.20 | Y | S | | | | | H | R | | R | | |
| ABE8.17 | | S | | | | | | | | R | | |
| pNMG- | Y | S | | | | | H | D | | R | | |
| B878 | | | | | | | | | | | | |
| pNMG- | Y | S | | | | | H | R | Y | R | | |
| B879 | | | | | | | | | | | | |
| pNMG- | Y | S | | | | | H | R | | R | I | |
| B880 | | | | | | | | | | | | |
| pNMG- | Y | S | | | | | H | R | | R | | N |
| B881 | | | | | | | | | | | | |
| pNMG- | Y | S | | | | | H | D | Y | R | I | N |
| B882 | | | | | | | | | | | | |
| pNMG- | Y | S | | R | N | | H | R | | R | | |
| B883 | | | | | | | | | | | | |
| pNMG- | Y | S | S | R | N | N | H | R | | R | | |
| B884 | | | | | | | | | | | | |
| pNMG- | Y | S | S | | | | H | R | | R | | |
| B885 | | | | | | | | | | | | |
| pNMG- | Y | S | | R | | | H | R | | R | | |
| B886 | | | | | | | | | | | | |
| pNMG- | Y | S | | | N | | H | R | | R | | |
| B887 | | | | | | | | | | | | |
| pNMG- | Y | S | | | | N | H | R | | R | | |
| B888 | | | | | | | | | | | | |
| pNMG- | Y | S | S | R | | | H | R | | R | | |
| B889 | | | | | | | | | | | | |
| pNMG- | Y | S | | | N | N | H | R | | R | | |
| B890 | | | | | | | | | | | | |
| pNMG- | Y | S | S | R | N | N | H | D | Y | R | I | N |
| B891 | | | | | | | | | | | | |

TABLE 5.2F
Base editor variants. Mutations are indicated with reference to ABE8.19m/8.20m.

| | AA positions: | | | | | | | | | | | | |
|---|---|---|---|---|---|---|---|---|---|---|---|---|---|
| | 17 | 27 | 48 | 49 | 76 | 82 | 84 | 118 | 142 | 147 | 149 | 166 | 167 |
| | ABE8.19m/8.20m | | | | | | | | | | | | |
| | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, and then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., et al. Science 337:816-821(2012), the entire contents of which is hereby incorporated by reference. Cas9 recognizes a short motif in the CRISPR repeat sequences (the PAM or protospacer adjacent motif) to help distinguish self versus non-self. See e.g., “Complete genome sequence of an M1 strain of Streptococcus pyogenes.” Ferretti, J. J. et al., Natl. Acad. Sci. U.S.A. 98:4658-4663(2001); “CRISPR RNA maturation by trans-encoded small RNA and host factor RNase III.” Deltcheva E. et al., Nature 471:602-607(2011); and “Programmable dual-RNA-guided DNA endonuclease in adaptive bacterial immunity.” Jinek M. et al, Science 337:816-821(2012), the entire contents of each of which are incorporated herein by reference).

The PAM sequence can be any PAM sequence known in the art. Suitable PAM sequences include, but are not limited to, NGG, NGA, NGC, NGN, NGT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

In an embodiment, a guide polynucleotide described herein can be RNA or DNA. In one embodiment, the guide polynucleotide is a guide RNA (gRNA). Exemplary guide RNA sequences are provided in Tables 1A, 1B, and 1D and exemplary spacer and target sequences are provided in Tables 1A, 1B, 1C, 1E, and in the Sequence Listing as SEQ ID NOs: 1214-2908, 403-412, and 435-446. An RNA/Cas complex can assist in “guiding” a Cas protein to a target DNA. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

In some embodiments, the guide polynucleotide is at least one tracrRNA. In some embodiments, the guide polynucleotide does not require PAM sequence to guide the polynucleotide-programmable DNA-binding domain (e.g., Cas9 or Cpf1) to the target nucleotide sequence.

A guide polynucleotide may include natural or non-natural (or unnatural) nucleotides (e.g., peptide nucleic acid or nucleotide analogs). In some cases, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length.

In some embodiments, the base editor provided herein utilizes one or more guide polynucleotide (e.g., multiple gRNA). In some embodiments, a single guide polynucleotide is utilized for different base editors described herein. For example, a single guide polynucleotide can be utilized for a cytidine base editor and an adenosine base editor.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in Tables 1A, 1B, and 1D and in the sequence listing as SEQ ID NOs: 317-327. Thus, a skilled artisan can change the genomic target of the Cas protein specificity is partially determined by how specific the gRNA targeting sequence is for the genomic target compared to the rest of the genome.

In other embodiments, a guide polynucleotide comprises both the polynucleotide targeting portion of the nucleic acid and the scaffold portion of the nucleic acid in a single molecule (i.e., a single-molecule guide nucleic acid). For example, a single-molecule guide polynucleotide can be a single guide RNA (sgRNA or gRNA). Herein the term guide polynucleotide sequence contemplates any single, dual or multi-molecule nucleic acid capable of interacting with and directing a base editor to a target polynucleotide sequence.

Typically, a guide polynucleotide (e.g., crRNA/trRNA complex or a gRNA) comprises a “polynucleotide-targeting segment” that includes a sequence capable of recognizing and binding to a target polynucleotide sequence, and a “protein-binding segment” that stabilizes the guide polynucleotide within a polynucleotide programmable nucleotide binding domain component of a base editor. In some embodiments, the polynucleotide targeting segment of the guide polynucleotide recognizes and binds to a DNA polynucleotide, thereby facilitating the editing of a base in DNA. In other cases, the polynucleotide targeting segment of the guide polynucleotide recognizes and binds to an RNA polynucleotide, thereby facilitating the editing of a base in RNA. Herein a “segment” refers to a section or region of a molecule, e.g., a contiguous stretch of nucleotides in the guide polynucleotide. A segment can also refer to a region/section of a complex such that a segment can comprise regions of more than one molecule. For example, where a guide polynucleotide comprises multiple nucleic acid molecules, the protein-binding segment of can include all or a portion (e.g., a functional portion) of multiple separate molecules that are for instance hybridized along a region of complementarity. In some embodiments, a protein-binding segment of a DNA-targeting RNA that comprises two separate molecules comprises (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and can include regions of RNA molecules that are of any total length and can include regions with complementarity to other molecules.

The guide polynucleotides can be synthesized chemically, synthesized enzymatically, or a combination thereof. For example, the gRNA can be synthesized using standard phosphoramidite-based solid-phase synthesis methods. Alternatively, the gRNA can be synthesized in vitro by operably linking DNA encoding the gRNA to a promoter control sequence that is recognized by a phage RNA polymerase. Examples of suitable phage promoter sequences include T7, T3, SP6 promoter sequences, or variations thereof. In embodiments in which the gRNA comprises two separate molecules (e.g., crRNA and tracrRNA), the crRNA can be chemically synthesized and the tracrRNA can be enzymatically synthesized.

A guide polynucleotide may be expressed, for example, by a DNA that encodes the gRNA, e.g., a DNA vector comprising a sequence encoding the gRNA. The gRNA may be encoded alone or together with an encoded base editor. Such DNA sequences may be introduced into an expression system, e.g., a cell, together or separately. For example, DNA sequences encoding a polynucleotide programmable nucleotide binding domain and a gRNA may be introduced into a cell, each DNA sequence can be part of a separate molecule (e.g., one vector containing the polynucleotide programmable nucleotide binding domain coding sequence and a second vector containing the gRNA coding sequence) or both can be part of a same molecule (e.g., one vector containing coding (and regulatory) sequence for both the polynucleotide programmable nucleotide binding domain and the gRNA). An RNA can be transcribed from a synthetic DNA molecule, e.g., a gBlocks® gene fragment. A gRNA molecule can be transcribed in vitro.

A gRNA or a guide polynucleotide can comprise three regions: a first region at the 5′ end that can be complementary to a target site in a chromosomal sequence, a second internal region that can form a stem loop structure, and a third 3′ region that does not form a secondary structure or bind a target site. A first region of each gRNA can also be different such that each gRNA guides a fusion protein or complex to a specific target site. Further, second and third regions of each gRNA can be identical in all gRNAs.

A first region of a gRNA or a guide polynucleotide can be complementary to sequence at a target site in a chromosomal sequence such that the first region of the gRNA can base pair with the target site. In some cases, a first region of a gRNA comprises from or from about 10 nucleotides to 25 nucleotides (i.e., from 10 nucleotides to nucleotides; or from about 10 nucleotides to about 25 nucleotides; or from 10 nucleotides to about 25 nucleotides; or from about 10 nucleotides to 25 nucleotides) or more. For example, a region of base pairing between a first region of a gRNA and a target site in a chromosomal sequence can be or can be about 10, 11, 12, 13, 14, 15, 16, 17, 18, 19, 20, 22, 23, 24, 25, or more nucleotides in length. Sometimes, a first region of a gRNA can be or can be about 19, 20, or 21 nucleotides in length.

A gRNA or a guide polynucleotide can also comprise a second region that forms a secondary structure. For example, a secondary structure formed by a gRNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A gRNA or a guide polynucleotide can also comprise a third region at the 3′ end that can be essentially single-stranded. For example, a third region is sometimes not complementarity to any chromosomal sequence in a cell of interest and is sometimes not complementarity to the rest of a gRNA. Further, the length of a third region can vary. A third region can be more than or more than about 4 nucleotides in length. For example, the length of a third region can range from or from about 5 to 60 nucleotides in length.

A gRNA or a guide polynucleotide can target any exon or intron of a gene target. In some cases, a guide can target exon 1 or 2 of a gene, in other cases; a guide can target exon 3 or 4 of a gene. In some embodiments, a composition comprises multiple gRNAs that all target the same exon or multiple gRNAs that target different exons. An exon and/or an intron of a gene can be targeted.

A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

Methods for selecting, designing, and validating guide polynucleotides, e.g., gRNAs and targeting sequences are described herein and known to those skilled in the art. For example, to minimize the impact of potential substrate promiscuity of a deaminase domain in the nucleobase editor system (e.g., an AID domain), the number of residues that could unintentionally be targeted for deamination (e.g., off-target C residues that could potentially reside on single strand DNA within the target nucleic acid locus) may be minimized. In addition, software tools can be used to optimize the gRNAs corresponding to a target nucleic acid sequence, e.g., to minimize total off-target activity across the genome. For example, for each possible targeting domain choice using S. pyogenes Cas9, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. First regions of gRNAs complementary to a target site can be identified, and all first regions (e.g., crRNAs) can be ranked according to its total predicted off-target score; the top-ranked targeting domains represent those that are likely to have the greatest on-target and the least off-target activity. Candidate targeting gRNAs can be functionally evaluated by using methods known in the art and/or as set forth herein.

As a non-limiting example, target DNA hybridizing sequences in crRNAs of a gRNA for use with Cas9s may be identified using a DNA sequence searching algorithm. gRNA design is carried out using custom gRNA design software based on the public tool cas-OFFinder as described in Bae S., Park J., & Kim J.-S. Cas-OFFinder: A fast and versatile algorithm that searches for potential off-target sites of Cas9 RNA-guided endonucleases. Bioinformatics 30, 1473-1475 (2014). This software scores guides after calculating their genome-wide off-target propensity. Typically matches ranging from perfect matches to 7 mismatches are considered for guides ranging in length from 17 to 24. Once the off-target sites are computationally-determined, an aggregate score is calculated for each guide and summarized in a tabular output using a web-interface. In addition to identifying potential target sites adjacent to PAM sequences, the software also identifies all PAM adjacent sequences that differ by 1, 2, 3 or more than 3 nucleotides from the selected target sites. Genomic DNA sequences for a target nucleic acid sequence, e.g., a target gene may be obtained and repeat elements may be screened using publicly available tools, for example, the RepeatMasker program. RepeatMasker searches input DNA sequences for repeated elements and regions of low complexity. The output is a detailed annotation of the repeats present in a given query sequence.

Following identification, first regions of gRNAs, e.g., crRNAs, are ranked into tiers based on their distance to the target site, their orthogonality and presence of 5′ nucleotides for close matches with relevant PAM sequences (for example, a 5′ G based on identification of close matches in the human genome containing a relevant PAM e.g., NGG PAM for S. pyogenes, NNGRRT or NNGRRV PAM for S. aureus). As used herein, orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality may be selected to minimize off-target DNA cleavage.

A gRNA can then be introduced into a cell or embryo as an RNA molecule or a non-RNA nucleic acid molecule, e.g., DNA molecule. In one embodiment, a DNA encoding a gRNA is operably linked to promoter control sequence for expression of the gRNA in a cell or embryo of interest. A RNA coding sequence can be operably linked to a promoter sequence that is recognized by RNA polymerase III (Pol III). Plasmid vectors that can be used to express gRNA include, but are not limited to, px330 vectors and px333 vectors. In some cases, a plasmid vector (e.g., px333 vector) comprises at least two gRNA-encoding DNA sequences. Further, a vector can comprise additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. A DNA molecule encoding a gRNA can also be linear. A DNA molecule encoding a gRNA or a guide polynucleotide can also be circular.

In some embodiments, a reporter system is used for detecting base-editing activity and testing candidate guide polynucleotides. In some embodiments, a reporter system comprises a reporter gene based assay where base editing activity leads to expression of the reporter gene. For example, a reporter system may include a reporter gene comprising a deactivated start codon, e.g., a mutation on the template strand from 3′-TAC-5′ to 3′-CAC-5′. Upon successful deamination of the target C, the corresponding mRNA will be transcribed as 5′-AUG-3′ instead of 5′-GUG-3′, enabling the translation of the reporter gene. Suitable reporter genes will be apparent to those of skill in the art. Non-limiting examples of reporter genes include gene encoding green fluorescence protein (GFP), red fluorescence protein (RFP), luciferase, secreted alkaline phosphatase (SEAP), or any other gene whose expression are detectable and apparent to those skilled in the art. The reporter system can be used to test many different gRNAs, e.g., in order to determine which residue(s) with respect to the target DNA sequence the respective deaminase will target. sgRNAs that target non-template strand can also be tested in order to assess off-target effects of a specific base editing protein, e.g., a Cas9 deaminase fusion protein or complex. In some embodiments, such gRNAs can be designed such that the mutated start codon will not be base-paired with the gRNA. The guide polynucleotides can comprise standard ribonucleotides, modified ribonucleotides (e.g., pseudouridine), ribonucleotide isomers, and/or ribonucleotide analogs. In some embodiments, the guide polynucleotide comprises at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and are preferably separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., Ni-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In a particular embodiment, the chemical modifications are 2′-O-methyl (2′-OMe) modifications. The modified guide RNAs may improve saCas9 efficacy and also specificity. The effect of an individual modification varies based on the position and combination of chemical modifications used as well as the inter- and intramolecular interactions with other modified nucleotides. By way of example, S-cEt has been used to improve oligonucleotide intramolecular folding.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises four modified nucleosides at the 5′ end and four modified nucleosides at the 3′ end of the guide. In some embodiments, the modified nucleoside comprises a 2′ O-methyl or a phosphorothioate.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. For such modifications, mN=2′-OMe; Ns=phosphorothioate (PS), where “N” represents the any nucleotide, as would be understood by one having skill in the art. In some cases, a nucleotide (N) may contain two modifications, for example, both a 2′-OMe and a PS modification. For example, a nucleotide with a phosphorothioate and 2′ OMe is denoted as “mNs;” when there are two modifications next to each other, the notation is “mNsmNs. In some embodiments of the modified gRNA, the gRNA comprises one or more chemical modifications selected from the group consisting of 2′-O-methyl (2′-OMe), phosphorothioate (PS), 2′-O-methyl thioPACE (MSP), 2′-O-methyl-PACE (MP), 2′-O-methyl thioPACE (MSP), 2′-fluoro RNA (2′-F-RNA), and constrained ethyl (S-cEt). In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

In some cases, a gRNA or a guide polynucleotide can comprise modifications. A modification can be made at any location of a gRNA or a guide polynucleotide. More than one modification can be made to a single gRNA or a guide polynucleotide. A gRNA or a guide polynucleotide can undergo quality control after a modification. In some cases, quality control can include PAGE, HPLC, MS, or any combination thereof.

A modification of a gRNA or a guide polynucleotide can be a substitution, insertion, deletion, chemical modification, physical modification, stabilization, purification, or any combination thereof.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a modification is permanent. In other cases, a modification is transient. In some cases, multiple modifications are made to a gRNA or a guide polynucleotide. A gRNA or a guide polynucleotide modification can alter physiochemical properties of a nucleotide, such as their conformation, polarity, hydrophobicity, chemical reactivity, base-pairing interactions, or any combination thereof.

A guide polynucleotide can be transferred into a cell by transfecting the cell with an isolated gRNA or a plasmid DNA comprising a sequence coding for the guide RNA and a promoter. A gRNA or a guide polynucleotide can also be transferred into a cell in other way, such as using virus-mediated gene delivery. A gRNA or a guide polynucleotide can be isolated. For example, a gRNA can be transfected in the form of an isolated RNA into a cell or organism. A gRNA can be prepared by in vitro transcription using any in vitro transcription system known in the art. A gRNA can be transferred to a cell in the form of isolated RNA rather than in the form of plasmid comprising encoding sequence for a gRNA.

A modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

In some embodiments, the guide RNA is designed such that base editing results in disruption of a splice site (i.e., a splice acceptor (SA) or a splice donor (SD). In some embodiments, the guide RNA is designed such that the base editing results in a premature STOP codon.

### Protospacer Adjacent Motif

The term “protospacer adjacent motif (PAM)” or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence is essential for target binding, but the exact sequence depends on a type of Cas protein. The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence. A PAM site is a nucleotide sequence in proximity to a target polynucleotide sequence. Some aspects of the disclosure provide for base editors comprising all or a portion (e.g., a functional portion) of CRISPR proteins that have different PAM specificities.

For example, typically Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. A PAM can be CRISPR protein-specific and can be different between different base editors comprising different CRISPR protein-derived domains. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 6 below.

TABLE 6
Cas9 proteins and corresponding
PAM sequences

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| Spy Mac | 5′-NAA-3′ |

In some embodiments, the PAM is NGC. In some embodiments, the NGC PAM is recognized by a Cas9 variant. In some embodiments, the NGC PAM Cas9 variant includes one or more amino acid substitutions selected from D1135M, S1136Q, G1218K, E1219F, A1322R, D1332A, R1335E, and T1337R (collectively termed “MQKFRAER”) of spCas9 (SEQ ID No: 197), or a corresponding mutation in another Cas9. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218). In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9. In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9.

In some embodiments, the PAM is NGT. In some embodiments, the NGT PAM is recognized by a Cas9 variant. In some embodiments, the Cas9 variant is generated through targeted mutations at one or more residues 1335, 1337, 1135, 1136, 1218, and/or 1219 of spCas9 (SEQ ID No: 197), or a corresponding mutation in another Cas9. In some embodiments, the NGT PAM Cas9 variant is created through targeted mutations at one or more residues 1219, 1335, 1337, 1218 of spCas9 (SEQ ID No: 197), or a corresponding mutation in another Cas9. In some embodiments, the NGT PAM Cas9 variant is created through targeted mutations at one or more residues 1135, 1136, 1218, 1219, and 1335 of spCas9 (SEQ ID No: 197, or a corresponding mutation in another Cas9. In some embodiments, the NGT PAM variant is selected from the set of targeted mutations provided in Tables 7A and 7B below.

TABLE 7A
NGT PAM Variant Mutations at residues 1219,
1335, 1337, 1218 of spCas9 (SEQ ID No: 197),
or a corresponding mutation in another Cas9

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 7B
NGT PAM Variant Mutations at residues 1135, 1136,
1218, 1219, and 1335 of spCas9 (SEQ ID No: 197),
or a corresponding mutation in another Cas9

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM Cas9 variant is selected from variant 5, 7, 28, 31, or 36 in Table 7A and Table 7B. In some embodiments, the variants have improved NGT PAM recognition.

In some embodiments, the NGT PAM Cas9 variants have mutations at residues 1219, 1335, 1337, and/or 1218. In some embodiments, the NGT PAM Cas9 variant is selected with mutations for improved recognition from the variants provided in Table 9 below.

TABLE 8
NGT PAM Variant Mutations at residues 1219,
1335, 1337, and 1218 of spCas9 (SEQ ID No: 197),
or a corresponding mutation in another Cas9

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, the NGT PAM is selected from the variants provided in Table 10 below.

TABLE 9
NGT PAM variants, where the amino acid residue locations are referenced to
of spCas9 (SEQ ID No: 197), or a corresponding mutation in another Cas9

| | NGTN | | | | | | | |
|---|---|---|---|---|---|---|---|---|
| | variant | D1135 | S1136 | G1218 | E1219 | A1322R | R1335 | T1337 |
| Variant 1 | LRKIQK | L | R | K | I | — | Q | K |
| Variant 2 | LRSVQK | L | R | S | V | — | Q | K |
| Variant 3 | LRSVQL | L | R | S | V | — | Q | L |
| Variant 4 | LRKIRQK | L | R | K | I | R | Q | K |
| Variant 5 | LRSVRQK | L | R | S | V | R | Q | K |
| Variant 6 | LRSVRQL | L | R | S | V | R | Q | L |

In some embodiments the NGTN Cas9 variant is variant 1. In some embodiments, the NGTN Cas9 variant is variant 2. In some embodiments, the NGTN variant is variant 3. In some embodiments, the NGTN Cas9 variant is variant 4. In some embodiments, the NGTN Cas9 variant is variant 5. In some embodiments, the NGTN Cas9 variant is variant 6.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein, wherein X is any amino acid except for D. In some embodiments, the SpCas9 comprises a D9A mutation, or a corresponding mutation in any of the amino acid sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having an NGG, a NGA, or a NGCG PAM sequence.

In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135E, R1335Q, and T1337R mutation, or a corresponding mutation in any of the amino acid sequences provided herein. In some embodiments, the SpCas9 domain comprises a D1135E, a R1335Q, and a T1337R mutation, or corresponding mutations in any of the amino acid sequences provided herein. In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135V, a R1335Q, and a T1337R mutation, or a corresponding mutation in any of the amino acid sequences provided herein. In some embodiments, the SpCas9 domain comprises a D1135V, a R1335Q, and a T1337R mutation, or corresponding mutations in any of the amino acid sequences provided herein. In some embodiments, the SpCas9 domain comprises one or more of a D1135X, a G1218X, a R1335X, and a T1337X mutation, or a corresponding mutation in any of the amino acid sequences provided herein, wherein X is any amino acid. In some embodiments, the SpCas9 domain comprises one or more of a D1135V, a G1218R, a R1335Q, and a T1337R mutation, or a corresponding mutation in any of the amino acid sequences provided herein. In some embodiments, the SpCas9 domain comprises a D1135V, a G1218R, a R1335Q, and a T1337R mutation, or corresponding mutations in any of the amino acid sequences provided herein.

In some examples, a PAM recognized by a CRISPR protein-derived domain of a base editor disclosed herein can be provided to a cell on a separate oligonucleotide to an insert (e.g., an AAV insert) encoding the base editor. In such embodiments, providing PAM on a separate oligonucleotide can allow cleavage of a target sequence that otherwise would not be able to be cleaved, because no adjacent PAM is present on the same polynucleotide as the target sequence.

In an embodiment, S. pyogenes Cas9 (SpCas9) can be used as a CRISPR endonuclease for genome engineering. However, others can be used. In some embodiments, a different endonuclease can be used to target certain genomic targets. In some embodiments, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4 kb coding sequence) can lead to plasmids carrying the SpCas9 cDNA that cannot be efficiently expressed in a cell. Conversely, the coding sequence for Staphylococcus aureus Cas9 (SaCas9) is approximately 1 kilobase shorter than SpCas9, possibly allowing it to be efficiently expressed in a cell. Similar to SpCas9, the SaCas9 endonuclease is capable of modifying target genes in mammalian cells in vitro and in mice in vivo. In some embodiments, a Cas protein can target a different PAM sequence. In some embodiments, a target gene can be adjacent to a Cas9 PAM, 5′-NGG, for example. In other embodiments, other Cas9 orthologs can have different PAM requirements. For example, other PAMs such as those of S. thermophilus (5′-NNAGAA for CRISPR1 and 5′-NGGNG for CRISPR3) and Neisseria meningitidis (5′-NNNNGATT) can also be found adjacent to a target gene.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some embodiments, an adjacent cut can be or can be about 3 base pairs upstream of a PAM. In some embodiments, an adjacent cut can be or can be about 10 base pairs upstream of a PAM. In some embodiments, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs. The sequences of exemplary SpCas9 proteins capable of binding a PAM sequence follow:

In some embodiments, engineered SpCas9 variants are capable of recognizing protospacer adjacent motif (PAM) sequences flanked by a 3′ H (non-G PAM) (see Tables 2A-2D). In some embodiments, the SpCas9 variants recognize NRNH PAMs (where R is A or G and H is A, C or T). In some embodiments, the non-G PAM is NRRH, NRTH, or NRCH (see e.g., Miller, S. M., et al. Continuous evolution of SpCas9 variants compatible with non-G PAMs, Nat. Biotechnol. (2020), the contents of which is incorporated herein by reference in its entirety).

In some embodiments, the Cas9 domain is a recombinant Cas9 domain. In some embodiments, the recombinant Cas9 domain is a SpyMacCas9 domain. In some embodiments, the SpyMacCas9 domain is a nuclease active SpyMacCas9, a nuclease inactive SpyMacCas9 (SpyMacCas9d), or a SpyMacCas9 nickase (SpyMacCas9n). In some embodiments, the SaCas9 domain, the SaCas9d domain, or the SaCas9n domain can bind to a nucleic acid sequence having a non-canonical PAM. In some embodiments, the SpyMacCas9 domain, the SpCas9d domain, or the SpCas9n domain can bind to a nucleic acid sequence having a NAA PAM sequence.

The sequence of an exemplary Cas9 A homolog of Spy Cas9 in Streptococcus macacae with native 5′-NAAN-3′ PAM specificity is known in the art and described, for example, by Chatterjee, et al., “A Cas9 with PAM recognition for adenine dinucleotides”, Nature Communications, vol. 11, article no. 2474 (2020), and is in the Sequence Listing as SEQ ID NO: 237.

In some embodiments, a variant Cas9 protein harbors, H840A, P475A, W476A, N477A, D1125A, W1126A, and D1218A mutations relative to a reference Cas9 sequence (e.g., spCas9 (SEQ ID No: 197)), or to a corresponding mutation in another Cas9, such that the polypeptide has a reduced ability to cleave a target DNA or RNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1218A mutations relative to a reference Cas9 sequence (e.g., spCas9 (SEQ ID No: 197)), or to a corresponding mutation in another Cas9, such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and W1126A mutations or when the variant Cas9 protein harbors P475A, W476A, N477A, D1125A, W1126A, and D1218A mutations relative to a reference Cas9 sequence (e.g., spCas9 (SEQ ID No: 197)), or to a corresponding mutation in another Cas9, the variant Cas9 protein does not bind efficiently to a PAM sequence. Thus, in some such cases, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted) relative to a reference Cas9 sequence (e.g., spCas9 (SEQ ID No: 197)), or to a corresponding mutation in another Cas9. Also, mutations other than alanine substitutions are suitable.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase or adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion protein comprises the following domains A-C, A-D, or A-E:
NH₂-[A-B—C]—COOH;
NH₂-[A-B—C-D]-COOH; or
NH₂-[A-B—C-D-E]-COOH;
   wherein A and C or A, C, and E, each comprises one or more of the following:
an adenosine deaminase domain or an active fragment thereof,
a cytidine deaminase domain or an active fragment thereof, and
wherein B or B and D, each comprises one or more domains having nucleic acid sequence specific binding activity.

In some embodiments, the fusion protein comprises the following structure:
NH2-[Aₙ-Bₒ—Cₙ]—COOH;
NH2-[Aₙ-Bₒ—Cₙ-Dₒ]—COOH; or
NH2-[Aₙ-Bₒ—Cₚ-Dₒ-E_{q}]—COOH;
   wherein A and C or A, C, and E, each comprises one or more of the following:
an adenosine deaminase domain or an active fragment thereof,
a cytidine deaminase domain or an active fragment thereof, and
   wherein n is an integer: 1, 2, 3, 4, or 5, wherein p is an integer: 0, 1, 2, 3, 4, or 5; wherein q is an integer 0, 1, 2, 3, 4, or 5; and wherein B or B and D each comprises a domain having nucleic acid sequence specific binding activity; and wherein o is an integer: 1, 2, 3, 4, or 5.

For example, and without limitation, in some embodiments, the fusion protein comprises the structure:
NH2-[adenosine deaminase]-[Cas9 domain]-COOH;
NH2-[Cas9 domain]-[adenosine deaminase]-COOH;
NH2-[cytidine deaminase]-[Cas9 domain]-COOH;
NH2-[Cas9 domain]-[cytidine deaminase]-COOH;
NH2-[cytidine deaminase]-[Cas9 domain]-[adenosine deaminase]-COOH;
NH2-[adenosine deaminase]-[Cas9 domain]-[cytidine deaminase]-COOH;
NH2-[adenosine deaminase]-[cytidine deaminase]-[Cas9 domain]-COOH;
NH2-[cytidine deaminase]-[adenosine deaminase]-[Cas9 domain]-COOH;
NH2-[Cas9 domain]-[adenosine deaminase]-[cytidine deaminase]-COOH; or
NH2-[Cas9 domain]-[cytidine deaminase]-[adenosine deaminase]-COOH.

In some embodiments, any of the Cas12 domains or Cas12 proteins provided herein may be fused with any of the cytidine or adenosine deaminases provided herein. For example, and without limitation, in some embodiments, the fusion protein comprises the structure:
NH2-[adenosine deaminase]-[Cas12 domain]-COOH;
NH2-[Cas12 domain]-[adenosine deaminase]-COOH;
NH2-[cytidine deaminase]-[Cas12 domain]-COOH;
NH2-[Cas12 domain]-[cytidine deaminase]-COOH;
NH2-[cytidine deaminase]-[Cas12 domain]-[adenosine deaminase]-COOH;
NH2-[adenosine deaminase]-[Cas12 domain]-[cytidine deaminase]-COOH;
NH2-[adenosine deaminase]-[cytidine deaminase]-[Cas12 domain]-COOH;
NH2-[cytidine deaminase]-[adenosine deaminase]-[Cas12 domain]-COOH;
NH2-[Cas12 domain]-[adenosine deaminase]-[cytidine deaminase]-COOH; or
NH2-[Cas12 domain]-[cytidine deaminase]-[adenosine deaminase]-COOH.

In some embodiments, the adenosine deaminase is a TadA*8. Exemplary fusion protein structures include the following:
NH2-[TadA*8]-[Cas9 domain]-COOH;
NH2-[Cas9 domain]-[TadA*8]—COOH;
NH2-[TadA*8]-[Cas12 domain]-COOH; or
NH2-[Cas12 domain]-[TadA*8]—COOH.

In some embodiments, the adenosine deaminase of the fusion protein or complex comprises a TadA*8 and a cytidine deaminase and/or an adenosine deaminase. In some embodiments, the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

Exemplary fusion protein structures include the following:
NH2-[TadA*8]-[Cas9/Cas12]-[adenosine deaminase]-COOH;
NH2-[adenosine deaminase]-[Cas9/Cas12]-[TadA*8]—COOH;
NH2-[TadA*8]-[Cas9/Cas12]-[cytidine deaminase]-COOH; or
NH2-[cytidine deaminase]-[Cas9/Cas12]-[TadA*8]—COOH.

In some embodiments, the adenosine deaminase of the fusion protein comprises a TadA*9 and a cytidine deaminase and/or an adenosine deaminase. Exemplary fusion protein structures include the following:
NH2-[TadA*9]-[Cas9/Cas12]-[adenosine deaminase]-COOH;
NH2-[adenosine deaminase]-[Cas9/Cas12]-[TadA*9]—COOH;
NH2-[TadA*9]-[Cas9/Cas12]-[cytidine deaminase]-COOH; or
NH2-[cytidine deaminase]-[Cas9/Cas12]-[TadA*9]—COOH.

In some embodiments, the fusion protein comprises a deaminase flanked by an N-terminal fragment and a C-terminal fragment of a Cas9 or a Cas12 polypeptide. In some embodiments, the fusion protein comprises a cytidine deaminase flanked by an N-terminal fragment and a C-terminal fragment of a Cas9 or a Cas12 polypeptide. In some embodiments, the fusion protein comprises an adenosine deaminase flanked by an N-terminal fragment and a C-terminal fragment of a Cas9 or Cas 12 polypeptide.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, the “-” used in the general architecture above indicates the optional presence of a linker. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, an NLS comprises the amino acid sequence

(SEQ ID NO: 328)
PKKKRKVEGADKRTADGSEFESPKKKRKV,
(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 329)
RKSGKIAAIVVKRPRKPKKKRKV,
or
(SEQ ID NO: 196)
MDSLLMNRRKFLYQFKNVRWAKGRRETYLC.

In some embodiments, the fusion proteins or complexes comprising a cytidine or adenosine deaminase, a Cas9 domain, and an NLS do not comprise a linker sequence. In some embodiments, linker sequences between one or more of the domains or proteins (e.g., cytidine or adenosine deaminase, Cas9 domain or NLS) are present. In some embodiments, a linker is present between the cytidine deaminase and adenosine deaminase domains and the napDNAbp. In some embodiments, the “-” used in the general architecture below indicates the optional presence of a linker. In some embodiments, the cytidine deaminase and adenosine deaminase and the napDNAbp are fused via any of the linkers provided herein. For example, in some embodiments the cytidine deaminase and adenosine deaminase and the napDNAbp are fused via any of the linkers provided herein.

In some embodiments, the general architecture of exemplary napDNAbp (e.g., Cas9 or Cas12) fusion proteins with a cytidine or adenosine deaminase and a napDNAbp (e.g. Cas9 or Cas12) domain comprises any one of the following structures, where NLS is a nuclear localization sequence (e.g., any NLS provided herein), NH2 is the N-terminus of the fusion protein, and COOH is the C-terminus of the fusion protein:
NH₂-NLS-[cytidine deaminase]-[napDNAbp domain]-COOH;
NH₂-NLS [napDNAbp domain]-[cytidine deaminase]-COOH;
NH₂-[cytidine deaminase]-[napDNAbp domain]-NLS—COOH;
NH₂-[napDNAbp domain]-[cytidine deaminase]-NLS—COOH;
NH₂-NLS-[adenosine deaminase]-[napDNAbp domain]-COOH;
NH₂-NLS [napDNAbp domain]-[adenosine deaminase]-COOH;
NH₂-[adenosine deaminase]-[napDNAbp domain]-NLS—COOH;
NH₂-[napDNAbp domain]-[adenosine deaminase]-NLS—COOH;
NH₂-NLS-[cytidine deaminase]-[napDNAbp domain]-[adenosine deaminase]-COOH;
NH₂-NLS-[adenosine deaminase]-[napDNAbp domain]-[cytidine deaminase]-COOH;
NH₂-NLS-[adenosine deaminase] [cytidine deaminase]-[napDNAbp domain]-COOH;
NH₂-NLS-[cytidine deaminase]-[adenosine deaminase]-[napDNAbp domain]-COOH;
NH₂-NLS-[napDNAbp domain]-[adenosine deaminase]-[cytidine deaminase]-COOH;
NH₂-NLS-[napDNAbp domain]-[cytidine deaminase]-[adenosine deaminase]-COOH;
NH₂-[cytidine deaminase]-[napDNAbp domain]-[adenosine deaminase]-NLS—COOH;
NH₂-[adenosine deaminase]-[napDNAbp domain]-[cytidine deaminase]-NLS—COOH;
NH₂-[adenosine deaminase] [cytidine deaminase]-[napDNAbp domain]-NLS—COOH;
NH₂-[cytidine deaminase]-[adenosine deaminase]-[napDNAbp domain]-NLS—COOH;
NH₂-[napDNAbp domain]-[adenosine deaminase]-[cytidine deaminase]-NLS—COOH; or
NH₂-[napDNAbp domain]-[cytidine deaminase]-[adenosine deaminase]-NLS—COOH.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR [PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the amino-terminus, about or more than about 1, 2, 3, 4, 5, 6, 7, 8, 9, 10 NLSs at or near the carboxy-terminus, or any combination thereof (e.g., one or more NLS at the amino-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise about 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises a uracil glycosylase inhibitor (UGI) domain. In some embodiments, cellular DNA repair response to the presence of U: G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

In some embodiments, a base editor comprises as a domain all or a portion (e.g., a functional portion) of a double-strand break (DSB) binding protein. For example, a DSB binding protein can include a Gam protein of bacteriophage Mu that can bind to the ends of DSBs and can protect them from degradation. See Komor, A. C., et al., “Improved base excision repair inhibition and bacteriophage Mu Gam protein yields C:G-to-T:A base editors with higher efficiency and product purity” Science Advances 3:eaao4774 (2017), the entire content of which is hereby incorporated by reference.

Additionally, in some embodiments, a Gam protein can be fused to an N terminus of a base editor. In some embodiments, a Gam protein can be fused to a C terminus of a base editor. The Gam protein of bacteriophage Mu can bind to the ends of double strand breaks (DSBs) and protect them from degradation. In some embodiments, using Gam to bind the free ends of DSB can reduce indel formation during the process of base editing. In some embodiments, 174-residue Gam protein is fused to the N terminus of the base editors. See Komor, A. C., et al., “Improved base excision repair inhibition and bacteriophage Mu Gam protein yields C:G-to-T:A base editors with higher efficiency and product purity” Science Advances 3:eaao4774 (2017). In some embodiments, a mutation or mutations can change the length of a base editor domain relative to a wild type domain. For example, a deletion of at least one amino acid in at least one domain can reduce the length of the base editor. In another case, a mutation or mutations do not change the length of a domain relative to a wild type domain. For example, substitutions in any domain does not change the length of the base editor.

Non-limiting examples of such base editors, where the length of all the domains is the same as the wild type domains, can include:
NH2-[nucleobase editing domain]-Linker1-[APOBEC1]-Linker2-[nucleobase editing domain]-COOH;
NH2-[nucleobase editing domain]-Linker1-[APOBEC1]-[nucleobase editing domain]-COOH;
NH2-[nucleobase editing domain]-[APOBEC1]-Linker2-[nucleobase editing domain]-COOH;
NH2-[nucleobase editing domain]-[APOBEC1]-[nucleobase editing domain]-COOH;
NH2-[nucleobase editing domain]-Linker1-[APOBEC1]-Linker2-[nucleobase editing domain]-[UGI]—COOH;
NH2-[nucleobase editing domain]-Linker1-[APOBEC1]-[nucleobase editing domain]-[UGI]—COOH;
NH2-[nucleobase editing domain]-[APOBEC1]-Linker2-[nucleobase editing domain]-[UGI]—COOH;
NH2-[nucleobase editing domain]-[APOBEC1]-[nucleobase editing domain]-[UGI]—COOH;
NH2-[UGI]-[nucleobase editing domain]-Linker1-[APOBEC1]-Linker2-[nucleobase editing domain]-COOH;
NH2-[UGI]-[nucleobase editing domain]-Linker1-[APOBEC1]-[nucleobase editing domain]-COOH;
NH2-[UGI]-[nucleobase editing domain]-[APOBEC1]-Linker2-[nucleobase editing domain]-COOH; or
NH2-[UGI]-[nucleobase editing domain]-[APOBEC1]-[nucleobase editing domain]-COOH.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

In some embodiments, a base editing system as provided herein provides a new approach to genome editing that uses a fusion protein or complex containing a catalytically defective Streptococcus pyogenes Cas9, a deaminase (e.g., cytidine or adenosine deaminase), and an inhibitor of base excision repair to induce programmable, single nucleotide (C→T or A→G) changes in DNA without generating double-strand DNA breaks, without requiring a donor DNA template, and without inducing an excess of stochastic insertions and deletions.

Details of nucleobase editing proteins are described in International PCT Application Nos. PCT/US2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleic acid (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine.

In some embodiments, a single guide polynucleotide may be utilized to target a deaminase to a target nucleic acid sequence. In some embodiments, a single pair of guide polynucleotides may be utilized to target different deaminases to a target nucleic acid sequence.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 opterator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

A base editor system may further comprise a guide polynucleotide component. It should be appreciated that components of the base editor system may be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the nucleobase editing component of the base editor system (e.g., the deaminase component) comprises an additional heterologous portion or domain (e.g., polynucleotide binding domain such as an RNA or DNA binding protein) that is capable of interacting with, associating with, or capable of forming a complex with a heterologous portion or segment (e.g., a polynucleotide motif), or antigen of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 opterator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some embodiments, a base editor system can further comprise an inhibitor of base excision repair (BER) component. It should be appreciated that components of the base editor system may be associated with each other via covalent bonds, noncovalent interactions, or any combination of associations and interactions thereof. The inhibitor of BER component may comprise a base excision repair inhibitor. In some embodiments, the inhibitor of base excision repair can be a uracil DNA glycosylase inhibitor (UGI). In some embodiments, the agent inhibiting the uracil-excision repair system is a uracil stabilizing protein (USP). In some embodiments, the inhibitor of base excision repair can be an inosine base excision repair inhibitor. In some embodiments, the inhibitor of base excision repair can be targeted to the target nucleotide sequence by the polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to an inhibitor of base excision repair. In some embodiments, a polynucleotide programmable nucleotide binding domain can be fused or linked to a deaminase domain and an inhibitor of base excision repair. In some embodiments, a polynucleotide programmable nucleotide binding domain can target an inhibitor of base excision repair to a target nucleotide sequence by non-covalently interacting with or associating with the inhibitor of base excision repair. For example, in some embodiments, the inhibitor of base excision repair component comprises an additional heterologous portion or domain that is capable of interacting with, associating with, or capable of forming a complex with a corresponding additional heterologous portion, antigen, or domain that is part of a polynucleotide programmable nucleotide binding domain. In some embodiments, the polynucleotide programming nucleotide binding domain component, and/or a guide polynucleotide (e.g., a guide RNA) complexed therewith, comprises an additional heterologous portion or domain that is capable of interacting with, associating with, or capable of forming a corresponding heterologous portion, antigen, or domain that is part of an inhibitor of base excision repair component. In some embodiments, the inhibitor of base excision repair can be targeted to the target nucleotide sequence by the guide polynucleotide. For example, in some embodiments, the inhibitor of base excision repair comprises an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain of the guide polynucleotide (e.g., polynucleotide binding domain such as an RNA or DNA binding protein) can be fused or linked to the inhibitor of base excision repair. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g. an MS2, an MS2 C-5 mutant, or an MS2 F-5 mutant), a non-natural RNA motif, a PP7 opterator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and VoB, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. Non-limiting examples of polypeptides that can dimerize and their corresponding dimerizing agents are provided in Table 10.1 below.

TABLE 10.1
Chemically induced dimerization systems.

| Dimerizing Polypeptides | | Dimerizing agent |
|---|---|---|
| FKBP | FKBP | FK1012 |
| FKBP | Calcineurin A (CNA) | FK506 |
| FKBP | CyP-Fas | FKCsA |
| FKBP | FRB (FKBP-rapamycin-binding) | Rapamycin |
| | domain of mTOR | |
| GyrB | GyrB | Coumermycin |
| GAI | GID1 (gibberellin insensitive | Gibberellin |
| | dwarf 1) | |
| ABI | PYL | Abscisic acid |
| ABI | PYRMandi | Mandipropamid |
| SNAP-tag | Halo Tag | HaXS |
| eDHFR | HaloTag | TMP-HTag |
| Bcl-xL | Fab (AZ1) | ABT-737 |

In embodiments, the additional heterologous portion is part of a guide RNA molecule. In some instances, the additional heterologous portion contains or is an RNA motif. The RNA motif may be positioned at the 5′ or 3′ end of the guide RNA molecule or various positions of a guide RNA molecule. In embodiments, the RNA motif is positioned within the guide RNA to reduce steric hindrance, optionally where such hindrance is associated with other bulky loops of an RNA scaffold. In some instances, it is advantageous to link the RNA motif is linked to other portions of the guide RNA by way of a linker, where the linker can be about, at least about, or no more than about 1, 2, 3, 4, 5, 6, 7, 8, 9, 10, or more nucleotides in length. Optionally, the linker contains a GC-rich nucleotide sequence. The guide RNA can contain 1, 2, 3, 4, 5, or more copies of the RNA motif, optionally where they are positioned consecutively, and/or optionally where they are each separated from one another by a linker(s). The RNA motif may include any one or more of the polynucleotide modifications described herein. Non-limiting examples of suitable modifications to the RNA motif include 2′ deoxy-2-aminopurine, 2′ribose-2-aminopurine, phosphorothioate mods, 2′-Omethyl mods, 2′-Fluro mods and LNA mods. Advantageously, the modifications help to increase stability and promote stronger bonds/folding structure of a hairpin(s) formed by the RNA motif.

In some embodiments, the RNA motif is modified to include an extension. In embodiments, the extension contains about, at least about, or no more than about 2, 3, 4, 5, 10, 15, 20, or 25 nucleotides. In some instances, the extension results in an alteration in the length of a stem formed by the RNA motif (e.g., a lengthening or a shortening). It can be advantageous for a stem formed by the RNA motif to be about, at least about, or no more than about 5, 10, 15, 20, 25, 30, 35, 40, 45, 50, 55, 60, 65, 70, 75, 80, 85, 90, 95, or 100 nucleotides in length. In various embodiments, the extension increases flexibility of the RNA motif and/or increases binding with a corresponding RNA motif.

In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease. In some embodiments, the base editor comprises nickase activity. In some embodiments, the intended edit of base pair is upstream of a PAM site. In some embodiments, the intended edit of base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edit of base-pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site.

In some embodiments, the method does not require a canonical (e.g., NGG) PAM site. In some embodiments, the nucleobase editor comprises a linker or a spacer. In some embodiments, the linker or spacer is 1-25 amino acids in length. In some embodiments, the linker or spacer is 5-20 amino acids in length. In some embodiments, the linker or spacer is 10, 11, 12, 13, 14, 15, 16, 17, 18, 19, or 20 amino acids in length.

In some embodiments, the base editing fusion proteins or complexes provided herein need to be positioned at a precise location, for example, where a target base is placed within a defined region (e.g., a “deamination window”). In some embodiments, a target can be within a 4 base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

In some embodiments, the target region comprises a target window, wherein the target window comprises the target nucleobase pair. In some embodiments, the target window comprises 1-10 nucleotides. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edit of base pair is within the target window. In some embodiments, the target window comprises the intended edit of base pair. In some embodiments, the method is performed using any of the base editors provided herein. In some embodiments, a target window is a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Other exemplary features that can be present in a base editor as disclosed herein are localization sequences, such as cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

In some embodiments, non-limiting exemplary cytidine base editors (CBE) include BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saB4E-Gam. BE4 extends the APOBEC1-Cas9n(D10A) linker to 32 amino acids and the Cas9n-UGI linker to 9 amino acids, and appends a second copy of UGI to the C-terminus of the construct with another 9-amino acid linker into a single base editor construct. The base editors saBE3 and saBE4 have the S. pyogenes Cas9n (D10A) replaced with the smaller S. aureus Cas9n(D10A). BE3-Gam, saBE3-Gam, BE4-Gam, and saBE4-Gam have 174 residues of Gam protein fused to the N-terminus of BE3, saBE3, BE4, and saBE4 via the 16 amino acid XTEN linker.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises evolved TadA variant. In some embodiments, the ABE is ABE 1.2 (TadA*-XTEN-nCas9-NLS). In some embodiments, TadA* comprises A106V and D108N mutations.

In some embodiments, the ABE is a second-generation ABE. In some embodiments, the ABE is ABE2.1, which comprises additional mutations D147Y and E155V in TadA* (TadA*2.1). In some embodiments, the ABE is ABE2.2, ABE2.1 fused to catalytically inactivated version of human alkyl adenine DNA glycosylase (AAG with E125Q mutation). In some embodiments, the ABE is ABE2.3, ABE2.1 fused to catalytically inactivated version of E. coli Endo V (inactivated with D35A mutation). In some embodiments, the ABE is ABE2.6 which has a linker twice as long (32 amino acids, (SGGS)₂ (SEQ ID NO: 330)-XTEN-(SGGS)₂ (SEQ ID NO: 330)) as the linker in ABE2.1. In some embodiments, the ABE is ABE2.7, which is ABE2.1 tethered with an additional wild-type TadA monomer. In some embodiments, the ABE is ABE2.8, which is ABE2.1 tethered with an additional TadA*2.1 monomer. In some embodiments, the ABE is ABE2.9, which is a direct fusion of evolved TadA (TadA*2.1) to the N-terminus of ABE2.1. In some embodiments, the ABE is ABE2.10, which is a direct fusion of wild-type TadA to the N-terminus of ABE2.1. In some embodiments, the ABE is ABE2.11, which is ABE2.9 with an inactivating E59A mutation at the N-terminus of TadA* monomer. In some embodiments, the ABE is ABE2.12, which is ABE2.9 with an inactivating E59A mutation in the internal TadA* monomer.

In some embodiments, the ABE is a third generation ABE. In some embodiments, the ABE is ABE3.1, which is ABE2.3 with three additional TadA mutations (L84F, H123Y, and 1156F).

In some embodiments, the ABE is a fourth generation ABE. In some embodiments, the ABE is ABE4.3, which is ABE3.1 with an additional TadA mutation A142N (TadA*4.3).

In some embodiments, the ABE is a fifth generation ABE. In some embodiments, the ABE is ABE5.1, which is generated by importing a consensus set of mutations from surviving clones (H36L, R51L, S146C, and K157N) into ABE3.1. In some embodiments, the ABE is ABE5.3, which has a heterodimeric construct containing wild-type E. coli TadA fused to an internal evolved TadA*. In some embodiments, the ABE is ABE5.2, ABE5.4, ABE5.5, ABE5.6, ABE5.7, ABE5.8, ABE5.9, ABE5.10, ABE5.11, ABE5.12, ABE5.13, or ABE5.14, as shown in Table 10 below. In some embodiments, the ABE is a sixth generation ABE. In some embodiments, the ABE is ABE6.1, ABE6.2, ABE6.3, ABE6.4, ABE6.5, or ABE6.6, as shown in Table 10 below. In some embodiments, the ABE is a seventh generation ABE. In some embodiments, the ABE is ABET.1, ABE7.2, ABE7.3, ABE7.4, ABE7.5, ABE7.6, ABE7.7, ABE7.8, ABE 7.9, or ABET.10, as shown in Table 10 below.

TABLE 10
Genotypes of ABEs

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| ABE0.1 | W | R | H | N | P | | R | N | L | S | A | D | H | G | A | S | D | R | E | I | K | K |
| ABE0.2 | W | R | H | N | P | | R | N | L | S | A | D | H | G | A | S | D | R | E | I | K | K |
| ABE1.1 | W | R | H | N | P | | R | N | L | S | A | N | H | G | A | S | D | R | E | I | K | K |
| ABE1.2 | W | R | H | N | P | | R | N | L | S | V | N | H | G | A | S | D | R | E | I | K | K |
| ABE2.1 | W | R | H | N | P | | R | N | L | S | V | N | H | G | A | S | Y | R | V | I | K | K |
| ABE2.2 | W | R | H | N | P | | R | N | L | S | V | N | H | G | A | S | Y | R | V | I | K | K |
| ABE2.3 | W | R | H | N | P | | R | N | L | S | V | N | H | G | A | S | Y | R | V | I | K | K |
| ABE2.4 | W | R | H | N | P | | R | N | L | S | V | N | H | G | A | S | Y | R | V | I | K | K |
| ABE2.5 | W | R | H | N | P | | R | N | L | S | V | N | H | G | A | S | Y | R | V | I | K | K |
| ABE2.6 | W | R | H | N | P | | R | N | L | S | V | N | H | G | A | S | Y | R | V | I | K | K |
| ABE2.7 | W | R | H | N | P | | R | N | L | S | V | N | H | G | A | S | Y | R | V | I | K | K |
| ABE2.8 | W | R | H | N | P | | R | N | L | S | V | N | H | G | A | S | Y | R | V | I | K | K |
| ABE2.9 | W | R | H | N | P | | R | N | L | S | V | N | H | G | A | S | Y | R | V | I | K | K |
| ABE2.10 | W | R | H | N | P | | R | N | L | S | V | N | H | G | A | S | Y | R | V | I | K | K |
| ABE2.11 | W | R | H | N | P | | R | N | L | S | V | N | H | G | A | S | Y | R | V | I | K | K |
| ABE2.12 | W | R | H | N | P | | R | N | L | S | V | N | H | G | A | S | Y | R | V | I | K | K |
| ABE3.1 | W | R | H | N | P | | R | N | F | S | V | N | Y | G | A | S | Y | R | V | F | K | K |
| ABE3.2 | W | R | H | N | P | | R | N | F | S | V | N | Y | G | A | S | Y | R | V | F | K | K |
| ABE3.3 | W | R | H | N | P | | R | N | F | S | V | N | Y | G | A | S | Y | R | V | F | K | K |
| ABE3.4 | W | R | H | N | P | | R | N | F | S | V | N | Y | G | A | S | Y | R | V | F | K | K |
| ABE3.5 | W | R | H | N | P | | R | N | F | S | V | N | Y | G | A | S | Y | R | V | F | K | K |
| ABE3.6 | W | R | H | N | P | | R | N | F | S | V | N | Y | G | A | S | Y | R | V | F | K | K |
| ABE3.7 | W | R | H | N | P | | R | N | F | S | V | N | Y | G | A | S | Y | R | V | F | K | K |
| ABE3.8 | W | R | H | N | P | | R | N | F | S | V | N | Y | G | A | S | Y | R | V | F | K | K |
| ABE4.1 | W | R | H | N | P | | R | N | L | S | V | N | H | G | N | S | Y | R | V | I | K | K |
| ABE4.2 | W | G | H | N | P | | R | N | L | S | V | N | H | G | N | S | Y | R | V | I | K | K |
| ABE4.3 | W | R | H | N | P | | R | N | F | S | V | N | Y | G | N | S | Y | R | V | F | K | K |
| ABE5.1 | W | R | L | N | P | | L | N | F | S | V | N | Y | G | A | C | Y | R | V | F | N | K |
| ABE5.2 | W | R | H | S | P | | R | N | F | S | V | N | Y | G | A | S | Y | R | V | F | K | T |
| ABE5.3 | W | R | L | N | P | | L | N | I | S | V | N | Y | G | A | C | Y | R | V | F | N | K |
| ABE5.4 | W | R | H | S | P | | R | N | F | S | V | N | Y | G | A | S | Y | R | V | F | K | T |
| ABE5.5 | W | R | L | N | P | | L | N | F | S | V | N | Y | G | A | C | Y | R | V | F | N | K |
| ABE5.6 | W | R | L | N | P | | L | N | F | S | V | N | Y | G | A | C | Y | R | V | F | N | K |
| ABE5.7 | W | R | L | N | P | | L | N | F | S | V | N | Y | G | A | C | Y | R | V | F | N | K |
| ABE5.8 | W | R | L | N | P | | L | N | F | S | V | N | Y | G | A | C | Y | R | V | F | N | K |
| ABE5.9 | W | R | L | N | P | | L | N | F | S | V | N | Y | G | A | C | Y | R | V | F | N | K |
| ABE5.10 | W | R | L | N | P | | L | N | F | S | V | N | Y | G | A | C | Y | R | V | F | N | K |
| ABE5.11 | W | R | L | N | P | | L | N | F | S | V | N | Y | G | A | C | Y | R | V | F | N | K |
| ABE5.12 | W | R | L | N | P | | L | N | F | S | V | N | Y | G | A | C | Y | R | V | F | N | K |
| ABE5.13 | W | R | H | N | P | | L | D | F | S | V | N | Y | A | A | S | Y | R | V | F | K | K |
| ABE5.14 | W | R | H | N | S | | L | N | F | C | V | N | Y | G | A | S | Y | R | V | F | K | K |
| ABE6.1 | W | R | H | N | S | | L | N | F | S | V | N | Y | G | N | S | Y | R | V | F | K | K |
| ABE6.2 | W | R | H | N | T | V | L | N | F | S | V | N | Y | G | N | S | Y | R | V | F | N | K |
| ABE6.3 | W | R | H | N | S | | L | N | F | S | V | N | Y | G | A | C | Y | R | V | F | N | K |
| ABE6.4 | W | R | L | N | S | | L | N | F | S | V | N | Y | G | N | C | Y | R | V | F | N | K |
| ABE6.5 | W | R | L | N | T | V | L | N | F | S | V | N | Y | G | A | C | Y | R | V | F | N | K |
| ABE6.6 | W | R | L | N | T | V | L | N | F | S | V | N | Y | G | N | C | Y | R | V | F | N | K |
| ABE7.1 | W | R | L | N | A | | L | N | F | S | V | N | Y | G | A | C | Y | R | V | F | N | K |
| ABE7.2 | W | R | L | N | A | | L | N | F | S | V | N | Y | G | N | C | Y | R | V | F | N | K |
| ABE7.3 | L | R | L | N | A | | L | N | F | S | V | N | Y | G | A | C | Y | R | V | F | N | K |
| ABE7.4 | R | R | L | N | A | | L | N | F | S | V | N | Y | G | A | C | Y | R | V | F | N | K |
| ABE7.5 | W | R | L | N | A | | L | N | F | S | V | N | Y | G | A | C | Y | H | V | F | N | K |
| ABE7.6 | W | R | L | N | A | | L | N | I | S | V | N | Y | G | A | C | Y | P | V | F | N | K |
| ABE7.7 | L | R | L | N | A | | L | N | F | S | V | N | Y | G | A | C | Y | P | V | F | N | K |
| ABE7.8 | L | R | L | N | A | | L | N | F | S | V | N | Y | G | A | C | Y | R | V | F | N | K |
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

In some embodiments, the base editor is an eighth generation ABE (ABE8). In some embodiments, the ABE8 contains a TadA*8 variant. In some embodiments, the ABE8 has a monomeric construct containing a TadA*8 variant (“ABE8.x-m”). In some embodiments, the ABE8 is ABE8.1-m, which has a monomeric construct containing TadA*7.10 with a Y147T mutation (TadA*8.1). In some embodiments, the ABE8 is ABE8.2-m, which has a monomeric construct containing TadA*7.10 with a Y147R mutation (TadA*8.2). In some embodiments, the ABE8 is ABE8.3-m, which has a monomeric construct containing TadA*7.10 with a Q154S mutation (TadA*8.3). In some embodiments, the ABE8 is ABE8.4-m, which has a monomeric construct containing TadA*7.10 with a Y123H mutation (TadA*8.4). In some embodiments, the ABE8 is ABE8.5-m, which has a monomeric construct containing TadA*7.10 with a V82S mutation (TadA*8.5). In some embodiments, the ABE8 is ABE8.6-m, which has a monomeric construct containing TadA*7.10 with a T166R mutation (TadA*8.6). In some embodiments, the ABE8 is ABE8.7-m, which has a monomeric construct containing TadA*7.10 with a Q154R mutation (TadA*8.7). In some embodiments, the ABE8 is ABE8.8-m, which has a monomeric construct containing TadA*7.10 with Y147R, Q154R, and Y123H mutations (TadA*8.8). In some embodiments, the ABE8 is ABE8.9-m, which has a monomeric construct containing TadA*7.10 with Y147R, Q154R and I76Y mutations (TadA*8.9). In some embodiments, the ABE8 is ABE8.10-m, which has a monomeric construct containing TadA*7.10 with Y147R, Q154R, and T166R mutations (TadA*8.10). In some embodiments, the ABE8 is ABE8.11-m, which has a monomeric construct containing TadA*7.10 with Y147T and Q154R mutations (TadA*8.11). In some embodiments, the ABE8 is ABE8.12-m, which has a monomeric construct containing TadA*7.10 with Y147T and Q154S mutations (TadA*8.12).

In some embodiments, the ABE8 is ABE8.13-m, which has a monomeric construct containing TadA*7.10 with Y123H (Y123H reverted from H123Y), Y147R, Q154R and I76Y mutations (TadA*8.13). In some embodiments, the ABE8 is ABE8.14-m, which has a monomeric construct containing TadA*7.10 with I76Y and V82S mutations (TadA*8.14). In some embodiments, the ABE8 is ABE8.15-m, which has a monomeric construct containing TadA*7.10 with V82S and Y147R mutations (TadA*8.15). In some embodiments, the ABE8 is ABE8.16-m, which has a monomeric construct containing TadA*7.10 with V82S, Y123H (Y123H reverted from H123Y) and Y147R mutations (TadA*8.16). In some embodiments, the ABE8 is ABE8.17-m, which has a monomeric construct containing TadA*7.10 with V82S and Q154R mutations (TadA*8.17). In some embodiments, the ABE8 is ABE8.18-m, which has a monomeric construct containing TadA*7.10 with V82S, Y123H (Y123H reverted from H123Y) and Q154R mutations (TadA*8.18). In some embodiments, the ABE8 is ABE8.19-m, which has a monomeric construct containing TadA*7.10 with V82S, Y123H (Y123H reverted from H123Y), Y147R and Q154R mutations (TadA*8.19). In some embodiments, the ABE8 is ABE8.20-m, which has a monomeric construct containing TadA*7.10 with I76Y, V82S, Y123H (Y123H reverted from H123Y), Y147R and Q154R mutations (TadA*8.20). In some embodiments, the ABE8 is ABE8.21-m, which has a monomeric construct containing TadA*7.10 with Y147R and Q154S mutations (TadA*8.21). In some embodiments, the ABE8 is ABE8.22-m, which has a monomeric construct containing TadA*7.10 with V82S and Q154S mutations (TadA*8.22). In some embodiments, the ABE8 is ABE8.23-m, which has a monomeric construct containing TadA*7.10 with V82S and Y123H (Y123H reverted from H123Y) mutations (TadA*8.23). In some embodiments, the ABE8 is ABE8.24-m, which has a monomeric construct containing TadA*7.10 with V82S, Y123H (Y123H reverted from H123Y), and Y147T mutations (TadA*8.24).

In some embodiments, the ABE8 has a heterodimeric construct containing wild-type E. coli TadA fused to a TadA*8 variant (“ABE8.x-d”). In some embodiments, the ABE8 is ABE8.1-d, which has a heterodimeric construct containing wild-type E. coli TadA fused to TadA*7.10 with a Y147T mutation (TadA*8.1). In some embodiments, the ABE8 is ABE8.2-d, which has a heterodimeric construct containing wild-type E. coli TadA fused to TadA*7.10 with a Y147R mutation (TadA*8.2). In some embodiments, the ABE8 is ABE8.3-d, which has a heterodimeric construct containing wild-type E. coli TadA fused to TadA*7.10 with a Q154S mutation (TadA*8.3). In some embodiments, the ABE8 is ABE8.4-d, which has a heterodimeric construct containing wild-type E. coli TadA fused to TadA*7.10 with a Y123H mutation (TadA*8.4). In some embodiments, the ABE8 is ABE8.5-d, which has a heterodimeric construct containing wild-type E. coli TadA fused to TadA*7.10 with a V82S mutation (TadA*8.5). In some embodiments, the ABE8 is ABE8.6-d, which has a heterodimeric construct containing wild-type E. coli TadA fused to TadA*7.10 with a T166R mutation (TadA*8.6). In some embodiments, the ABE8 is ABE8.7-d, which has a heterodimeric construct containing wild-type E. coli TadA fused to TadA*7.10 with a Q154R mutation (TadA*8.7). In some embodiments, the ABE8 is ABE8.8-d, which has a heterodimeric construct containing wild-type E. coli TadA fused to TadA*7.10 with Y147R, Q154R, and Y123H mutations (TadA*8.8). In some embodiments, the ABE8 is ABE8.9-d, which has a heterodimeric construct containing wild-type E. coli TadA fused to TadA*7.10 with Y147R, Q154R and I76Y mutations (TadA*8.9). In some embodiments, the ABE8 is ABE8.10-d, which has a heterodimeric construct containing wild-type E. coli TadA fused to TadA*7.10 with Y147R, Q154R, and T166R mutations (TadA*8.10). In some embodiments, the ABE8 is ABE8.11-d, which has a heterodimeric construct containing wild-type E. coli TadA fused to TadA*7.10 with Y147T and Q154R mutations (TadA*8.11). In some embodiments, the ABE8 is ABE8.12-d, which has heterodimeric construct containing wild-type E. coli TadA fused to TadA*7.10 with Y147T and Q154S mutations (TadA*8.12). In some embodiments, the ABE8 is ABE8.13-d, which has a heterodimeric construct containing wild-type E. coli TadA fused to TadA*7.10 with Y123H (Y123H reverted from H123Y), Y147R, Q154R and I76Y mutations (TadA*8.13). In some embodiments, the ABE8 is ABE8.14-d, which has a heterodimeric construct containing wild-type E. coli TadA fused to TadA*7.10 with 176Y and V82S mutations (TadA*8.14). In some embodiments, the ABE8 is ABE8.15-d, which has a heterodimeric construct containing wild-type E. coli TadA fused to TadA*7.10 with V82S and Y147R mutations (TadA*8.15). In some embodiments, the ABE8 is ABE8.16-d, which has a heterodimeric construct containing wild-type E. coli TadA fused to TadA*7.10 with V82S, Y123H (Y123H reverted from H123Y) and Y147R mutations (TadA*8.16). In some embodiments, the ABE8 is ABE8.17-d, which has a heterodimeric construct containing wild-type E. coli TadA fused to TadA*7.10 with V82S and Q154R mutations (TadA*8.17). In some embodiments, the ABE8 is ABE8.18-d, which has a heterodimeric construct containing wild-type E. coli TadA fused to TadA*7.10 with V82S, Y123H (Y123H reverted from H123Y) and Q154R mutations (TadA*8.18). In some embodiments, the ABE8 is ABE8.19-d, which has a heterodimeric construct containing wild-type E. coli TadA fused to TadA*7.10 with V82S, Y123H (Y123H reverted from H123Y), Y147R and Q154R mutations (TadA*8.19). In some embodiments, the ABE8 is ABE8.20-d, which has a heterodimeric construct containing wild-type E. coli TadA fused to TadA*7.10 with 176Y, V82S, Y123H (Y123H reverted from H123Y), Y147R and Q154R mutations (TadA*8.20). In some embodiments, the ABE8 is ABE8.21-d, which has a heterodimeric construct containing wild-type E. coli TadA fused to TadA*7.10 with Y147R and Q154S mutations (TadA*8.21). In some embodiments, the ABE8 is ABE8.22-d, which has a heterodimeric construct containing wild-type E. coli TadA fused to TadA*7.10 with V82S and Q154S mutations (TadA*8.22). In some embodiments, the ABE8 is ABE8.23-d, which has a heterodimeric construct containing wild-type E. coli TadA fused to TadA*7.10 with V82S and Y123H (Y123H reverted from H123Y) mutations (TadA*8.23). In some embodiments, the ABE8 is ABE8.24-d, which has a heterodimeric construct containing wild-type E. coli TadA fused to TadA*7.10 with V82S, Y123H (Y123H reverted from H123Y), and Y147T mutations (TadA*8.24).

In some embodiments, the ABE8 has a heterodimeric construct containing TadA*7.10 fused to a TadA*8 variant (“ABE8.x-7”). In some embodiments, the ABE8 is ABE8.1-7, which has a heterodimeric construct containing TadA*7.10 fused to TadA*7.10 with a Y147T mutation (TadA*8.1). In some embodiments, the ABE8 is ABE8.2-7, which has a heterodimeric construct containing TadA*7.10 fused to TadA*7.10 with a Y147R mutation (TadA*8.2). In some embodiments, the ABE8 is ABE8.3-7, which has a heterodimeric construct containing TadA*7.10 fused to TadA*7.10 with a Q154S mutation (TadA*8.3). In some embodiments, the ABE8 is ABE8.4-7, which has a heterodimeric construct containing TadA*7.10 fused to TadA*7.10 with a Y123H mutation (TadA*8.4). In some embodiments, the ABE8 is ABE8.5-7, which has a heterodimeric construct containing TadA*7.10 fused to TadA*7.10 with a V82S mutation (TadA*8.5). In some embodiments, the ABE8 is ABE8.6-7, which has a heterodimeric construct containing TadA*7.10 fused to TadA*7.10 with a T166R mutation (TadA*8.6). In some embodiments, the ABE8 is ABE8.7-7, which has a heterodimeric construct containing TadA*7.10 fused to TadA*7.10 with a Q154R mutation (TadA*8.7). In some embodiments, the ABE8 is ABE8.8-7, which has a heterodimeric construct containing TadA*7.10 fused to TadA*7.10 with Y147R, Q154R, and Y123H mutations (TadA*8.8). In some embodiments, the ABE8 is ABE8.9-7, which has a heterodimeric construct containing TadA*7.10 fused to TadA*7.10 with Y147R, Q154R and I76Y mutations (TadA*8.9). In some embodiments, the ABE8 is ABE8.10-7, which has a heterodimeric construct containing TadA*7.10 fused to TadA*7.10 with Y147R, Q154R, and T166R mutations (TadA*8.10). In some embodiments, the ABE8 is ABE8.11-7, which has a heterodimeric construct containing TadA*7.10 fused to TadA*7.10 with Y147T and Q154R mutations (TadA*8.11). In some embodiments, the ABE8 is ABE8.12-7, which has a heterodimeric construct containing TadA*7.10 fused to TadA*7.10 with Y147T and Q154S mutations (TadA*8.12). In some embodiments, the ABE8 is ABE8.13-7, which has a heterodimeric construct containing TadA*7.10 fused to TadA*7.10 with Y123H (Y123H reverted from H123Y), Y147R, Q154R and I76Y mutations (TadA*8.13). In some embodiments, the ABE8 is ABE8.14-7, which has a heterodimeric construct containing TadA*7.10 fused to TadA*7.10 with I76Y and V82S mutations (TadA*8.14). In some embodiments, the ABE8 is ABE8.15-7, which has a heterodimeric construct containing TadA*7.10 fused to TadA*7.10 with V82S and Y147R mutations (TadA*8.15). In some embodiments, the ABE8 is ABE8.16-7, which has a heterodimeric construct containing TadA*7.10 fused to TadA*7.10 with V82S, Y123H (Y123H reverted from H123Y) and Y147R mutations (TadA*8.16). In some embodiments, the ABE8 is ABE8.17-7, which has a heterodimeric construct containing TadA*7.10 fused to TadA*7.10 with V82S and Q154R mutations (TadA*8.17). In some embodiments, the ABE8 is ABE8.18-7, which has a heterodimeric construct containing TadA*7.10 fused to TadA*7.10 with V82S, Y123H (Y123H reverted from H123Y) and Q154R mutations (TadA*8.18). In some embodiments, the ABE8 is ABE8.19-7, which has a heterodimeric construct containing TadA*7.10 fused to TadA*7.10 with V82S, Y123H (Y123H reverted from H123Y), Y147R and Q154R mutations (TadA*8.19). In some embodiments, the ABE8 is ABE8.20-7, which has a heterodimeric construct containing TadA*7.10 fused to TadA*7.10 with I76Y, V82S, Y123H (Y123H reverted from H123Y), Y147R and Q154R mutations (TadA*8.20). In some embodiments, the ABE8 is ABE8.21-7, which has a heterodimeric construct containing TadA*7.10 fused to TadA*7.10 with Y147R and Q154S mutations (TadA*8.21). In some embodiments, the ABE8 is ABE8.22-7, which has a heterodimeric construct containing TadA*7.10 fused to TadA*7.10 with V82S and Q154S mutations (TadA*8.22). In some embodiments, the ABE8 is ABE8.23-7, which has a heterodimeric construct containing TadA*7.10 fused to TadA*7.10 with V82S and Y123H (Y123H reverted from H123Y) mutations (TadA*8.23). In some embodiments, the ABE8 is ABE8.24-7, which has a heterodimeric construct containing TadA*7.10 fused to TadA*7.10 with V82S, Y123H (Y123H reverted from H123Y), and Y147T mutations (TadA*8.24

In some embodiments, the ABE is ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, or ABE8.24-d as shown in Table 11 below.

TABLE 11
Adenosine Base Editor 8 (ABE8) Variants

| ABE8 | Adenosine Deaminase | Adenosine Deaminase Description |
|---|---|---|
| ABE8.1-m | TadA*8.1 | Monomer_TadA*7.10 + Y147T |
| ABE8.2-m | TadA*8.2 | Monomer_TadA*7.10 + Y147R |
| ABE8.3-m | TadA*8.3 | Monomer_TadA*7.10 + Q154S |
| ABE8.4-m | TadA*8.4 | Monomer_TadA*7.10 + Y123H |
| ABE8.5-m | TadA*8.5 | Monomer_TadA*7.10 + V82S |
| ABE8.6-m | TadA*8.6 | Monomer_TadA*7.10 + T166R |
| ABE8.7-m | TadA*8.7 | Monomer_TadA*7.10 + Q154R |
| ABE8.8-m | TadA*8.8 | Monomer_TadA*7.10 + Y147R_Q154R_Y123H |
| ABE8.9-m | TadA*8.9 | Monomer_TadA*7.10 + Y147R_Q154R_I76Y |
| ABE8.10-m | TadA*8.10 | Monomer_TadA*7.10 + Y147R_Q154R_T166R |
| ABE8.11-m | TadA*8.11 | Monomer_TadA*7.10 + Y147T_Q154R |
| ABE8.12-m | TadA*8.12 | Monomer_TadA*7.10 + Y147T_Q154S |
| ABE8.13-m | TadA*8.13 | Monomer_TadA*7.10 + |
| | | Y123H_Y147R_Q154R_I76Y |
| ABE8.14-m | TadA*8.14 | Monomer_TadA*7.10 + I76Y_V82S |
| ABE8.15-m | TadA*8.15 | Monomer_TadA*7.10 + V82S_Y147R |
| ABE8.16-m | TadA*8.16 | Monomer_TadA*7.10 + V82S_Y123H_Y147R |
| ABE8.17-m | TadA*8.17 | Monomer_TadA*7.10 + V82S_Q154R |
| ABE8.18-m | TadA*8.18 | Monomer_TadA*7.10 + V82S_Y123H_Q154R |
| ABE8.19-m | TadA*8.19 | Monomer_TadA*7.10 + |
| | | V82S_Y123H_Y147R_Q154R |
| ABE8.20-m | TadA*8.20 | Monomer_TadA*7.10 + |
| | | I76Y_V82S_Y123H_Y147R_Q154R |
| ABE8.21-m | TadA*8.21 | Monomer_TadA*7.10 + Y147R_Q154S |
| ABE8.22-m | TadA*8.22 | Monomer_TadA*7.10 + V82S_Q154S |
| ABE8.23-m | TadA*8.23 | Monomer_TadA*7.10 + V82S Y123H |
| ABE8.24-m | TadA*8.24 | Monomer_TadA*7.10 + V82S_Y123H_Y147T |
| ABE8.1-d | TadA*8.1 | Heterodimer_(WT) + (TadA*7.10 + Y147T) |
| ABE8.2-d | TadA*8.2 | Heterodimer_(WT) + (TadA*7.10 + Y147R) |
| ABE8.3-d | TadA*8.3 | Heterodimer_(WT) + (TadA*7.10 + Q154S) |
| ABE8.4-d | TadA*8.4 | Heterodimer_(WT) + (TadA*7.10 + Y123H) |
| ABE8.5-d | TadA*8.5 | Heterodimer_(WT) + (TadA*7.10 + V82S) |
| ABE8.6-d | TadA*8.6 | Heterodimer_(WT) + (TadA*7.10 + T166R) |
| ABE8.7-d | TadA*8.7 | Heterodimer_(WT) + (TadA*7.10 + Q154R) |
| ABE8.8-d | TadA*8.8 | Heterodimer_(WT) + (TadA*7.10 + |
| | | Y147R_Q154R_Y123H) |
| ABE8.9-d | TadA*8.9 | Heterodimer (WT) + (TadA*7.10 + |
| | | Y147R_Q154R_I76Y) |
| ABE8.10-d | TadA*8.10 | Heterodimer_(WT) + (TadA*7.10 + |
| | | Y147R_Q154R_T166R) |
| ABE8.11-d | TadA*8.11 | Heterodimer_(WT) + (TadA*7.10 + Y147T_Q154R) |
| ABE8.12-d | TadA*8.12 | Heterodimer_(WT) + (TadA*7.10 + Y147T_Q154S) |
| ABE8.13-d | TadA*8.13 | Heterodimer_(WT) + (TadA*7.10 + |
| | | Y123H_Y147T_Q154R_I76Y) |
| ABE8.14-d | TadA*8.14 | Heterodimer_(WT) + (TadA*7.10 + I76Y_V82S) |
| ABE8.15-d | TadA*8.15 | Heterodimer_(WT) + (TadA*7.10 + V82S_Y147R) |
| ABE8.16-d | TadA*8.16 | Heterodimer_(WT) + (TadA*7.10 + |
| | | V82S_Y123H_Y147R) |
| ABE8.17-d | TadA*8.17 | Heterodimer_(WT) + (TadA*7.10 + V82S_Q154R) |
| ABE8.18-d | TadA*8.18 | Heterodimer_(WT) + (TadA*7.10 + |
| | | V82S_Y123H_Q154R) |
| ABE8.19-d | TadA*8.19 | Heterodimer_(WT) + (TadA*7.10 + |
| | | V82S_Y123H_Y147R_Q154R) |
| ABE8.20-d | TadA*8.20 | Heterodimer (WT) + (TadA*7.10 + |
| | | I76Y_V82S_Y123H_Y147R_Q154R) |
| ABE8.21-d | TadA*8.21 | Heterodimer_(WT) + (TadA*7.10 + Y147R_Q154S) |
| ABE8.22-d | TadA*8.22 | Heterodimer_(WT) + (TadA*7.10 + V82S_Q154S) |
| ABE8.23-d | TadA*8.23 | Heterodimer_(WT) + (TadA*7.10 + V82S_Y123H) |
| ABE8.24-d | TadA*8.24 | Heterodimer_(WT) + (TadA*7.10 + |
| | | V82S_Y123H_Y147T) |

In some embodiments, the ABE8 is ABE8a-m, which has a monomeric construct containing TadA*7.10 with R26C, A1U09S, T111R, D119N, H122N, Y1471D, F149Y, T1661, and D167N mutations (TadA*8a). In some embodiments, the ABE8 is ABE8b-m, which has a monomeric construct containing TadA*7.10 with V88A, A109S, T111R, D119N, H122N, F149Y, T1661, and D167N mutations (TadA*8b). In some embodiments, the ABE8 is ABE8c-m, which has a monomeric construct containing TadA*7.10 with R26C, A109S, T111R, D119N, H122N, F149Y, T1661, and D167N mutations (TadA*8c). In some embodiments, the ABE8 is ABE8d-m, which has a monomeric construct containing TadA*7.10 with V88A, T111R, D119N, and F149Y mutations (TadA*8d). In some embodiments, the ABE8 is ABE8e-m, which has a monomeric construct containing TadA*7.10 with A109S, T111R, D119N, H122N, Y147D, F149Y, T1661, and D167N mutations (TadA*8e).

In some embodiments, the ABE8 is ABE8a-d, which has a heterodimeric construct containing wild-type E. coli TadA fused to TadA*7.10 with R26C, A109S, T111R, D119, H122N, Y147D, F149Y, T1661, and D167N mutations (TadA*8a). In some embodiments, the ABE8 is ABE8b-d, which has a heterodimeric construct containing wild-type E. coli TadA fused to TadA*7.10 with V88A, A109S, T111R, D119N, H122N, F149Y, T1661, and D167N mutations (TadA*8b). In some embodiments, the ABE8 is ABE8c-d, which has a heterodimeric construct containing wild-type E. coli TadA fused to TadA*7.10 with R26C, A109S, T111R, D119N, H122N, F149Y, T1661, and D167N mutations (TadA*8c). In some embodiments, the ABE8 is ABE8d-d, which has a heterodimeric construct containing wild-type E. coli TadA fused to TadA*7.10 with V88A, T111R, D119N, and F149Y mutations (TadA*8d). In some embodiments, the ABE8 is ABE8e-d, which has a heterodimeric construct containing wild-type E. coli TadA fused to TadA*7.10 with A109S, T111R, D119N, H122N, Y147D, F149Y, T1661, and D167N mutations (TadA*8e).

In some embodiments, the ABE8 is ABE8a-7, which has a heterodimeric construct containing TadA*7.10 fused to TadA*7.10 with R26C, A109S, T111R, D119, H122N, Y147D, F149Y, T1661, and D167N mutations (TadA*8a). In some embodiments, the ABE8 is ABE8b-7, which has a heterodimeric construct containing TadA*7.10 fused to TadA*7.10 with V88A, A109S, T111R, Di 19N, H122N, F149Y, T166I, and D167N mutations (TadA*8b). In some embodiments, the ABE8 is ABE8c-7, which has a heterodimeric construct containing TadA*7.10 fused to TadA*7.10 with R26C, A109S, T111R, D119N, H122N, F149Y, T1661, and D167N mutations (TadA*8c). In some embodiments, the ABE8 is ABE8d-7, which has a heterodimeric construct containing TadA*7.10 fused to TadA*7.10 with V88A, T111R, D119N, and F149Y mutations (TadA*8d). In some embodiments, the ABE8 is ABE8e-7, which has a heterodimeric construct containing TadA*7.10 fused to TadA*7.10 with A109S, T111R, D119N, H122N, Y147D, F149Y, T1661, and D167N mutations (TadA*8e).

In some embodiments, the ABE is ABE8a-m, ABE8b-m, ABE8c-m, ABE8d-m, ABE8e-m, ABE8a-d, ABE8b-d, ABE8c-d, ABE8d-d, or ABE8e-d, as shown in Table 12 below. In some embodiments, the ABE is ABE8e-m or ABE8e-d. ABE8e shows efficient adenine base editing activity and low indel formation when used with Cas homologues other than SpCas9, for example, SaCas9, SaCas9-KKH, Cas12a homologues, e.g., LbCas12a, enAs-Cas12a, SpCas9-NG and circularly permuted CP1028-SpCas9 and CP1041-SpCas9. In addition to the mutations shown for ABE8e in Table 12, off-target RNA and DNA editing were reduced by introducing a V106W substitution into the TadA domain (as described in M. Richter et al., 2020, Nature Biotechnology, doi.org/10.1038/s4i587-020-0453-z, the entire contents of which are incorporated by reference herein).

TABLE 12
Additional Adenosine Base Editor 8 Variants. In the table, “monomer”
indicates an ABE comprising a single TadA*7.10 comprising the indicated alterations
and “heterodimer” indicates an ABE comprising a TadA*7.10 comprising the indicated
alterations fused to an E. coli TadA adenosine deaminase.

| ABE8 Base | Adenosine | |
|---|---|---|
| Editor | Deaminase | Adenosine Deaminase Description |
| ABE8a-m | TadA*8a | Monomer_TadA*7.10 + R26C + A109S + T111R + D119N + |
| | | H122N + Y147D + F149Y + T166I + D167N |
| ABE8b-m | TadA*8b | Monomer_TadA*7.10 + V88A + A109S + T111R + D119N + |
| | | H122N + F149Y + T166I + D167N |
| ABE8c-m | TadA*8c | Monomer_TadA*7.10 + R26C + A109S + T111R + D119N + |
| | | H122N + F149Y + T166I + D167N |
| ABE8d-m | TadA*8d | Monomer_TadA*7.10 + V88A + T111R + D119N + F149Y |
| ABE8e-m | TadA*8e | Monomer_TadA*7.10 + A109S + T111R + D119N + H122N + |
| | | Y147D + F149Y + T166I + D167N |
| ABE8a-d | TadA*8a | Heterodimer_(WT) + (TadA*7.10 + R26C + A109S + T111R + |
| | | D119N + H122N + Y147D + F149Y + T166I + D167N) |
| ABE8b-d | TadA*8b | Heterodimer_(WT) + (TadA*7.10 + V88A + A109S + T111R + |
| | | D119N + H122N + F149Y + T166I + D167N) |
| ABE8c-d | TadA*8c | Heterodimer_(WT) + (TadA*7.10 + R26C + A109S + T111R + |
| | | D119N + H122N + F149Y + T166I + D167N) |
| ABE8d-d | TadA*8d | Heterodimer_(WT) + (TadA*7.10 + V88A + T111R + D119N + |
| | | F149Y) |
| ABE8e-d | TadA*8e | Heterodimer_(WT) + (TadA*7.10 + A109S + T111R + D119N + |
| | | H122N + Y147D + F149Y + T166I + D167N) |

In some embodiments, base editors (e.g., ABE8) are generated by cloning an PP194 adenosine deaminase variant (e.g., TadA*8) into a scaffold that includes a circular permutant Cas9 (e.g., CP5 or CP6) and a bipartite nuclear localization sequence. In some embodiments, the base editor (e.g., ABE7.9, ABE7.10, or ABE8) is an NGC PAM CP5 variant (S. pyogenes Cas9 or spVRQR Cas9). In some embodiments, the base editor (e.g., ABE7.9, ABE7.10, or ABE8) is an AGA PAM CP5 variant (S. pyogenes Cas9 or spVRQR Cas9). In some embodiments, the base editor (e.g, ABE7.9, ABE7.10, or ABE8) is an NGC PAM CP6 variant (S. pyogenes Cas9 or spVRQR Cas9). In some embodiments, the base editor (e.g. ABE7.9, ABET.10, or ABE8) is an AGA PAM CP6 variant (S. pyogenes Cas9 or spVRQR Cas9).

In some embodiments, the ABE has a genotype as shown in Table 13 below.

TABLE 13
Genotypes of ABEs

| | | | | | | | | | | | | | | | | | | | | | |
|---|---|---|---|---|---|---|---|---|---|---|---|---|---|---|---|---|---|---|---|---|---|
| 23 | 26 | 36 | 37 | 48 | 49 | 51 | 172 | 84 | 87 | 105 | 108 | 123 | 125 | 142 | 145 | 147 | 152 | 155 | 156 | 157 | 161 |

| | | | | | | | | | | | | | | | | | | | | | |
|---|---|---|---|---|---|---|---|---|---|---|---|---|---|---|---|---|---|---|---|---|---|
| ABE7.9 | L | R | L | N | A | L | N | F | S | V | N | Y | G | N | C | Y | P | V | F | N | K |
| ABE7.10 | R | R | L | N | A | L | N | F | S | V | N | Y | G | A | C | Y | P | V | F | N | K |

As shown in Table 14 below, genotypes of 40 ABE8s are described. Residue positions in the evolved E. coli TadA portion of ABE are indicated. Mutational changes in ABE8 are shown when distinct from ABE7.10 mutations. In some embodiments, the ABE has a genotype of one of the ABEs as shown in Table 14 below.

TABLE 14
Residue Identity in Evolved TadA

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| ABE7.10 | R | L | A | L | I | V | F | V | N | Y | C | Y | P | Q | V | F | N | T |
| ABE8.1-m | | | | | | | | | | | | T | | | | | | |
| ABE8.2-m | | | | | | | | | | | | R | | | | | | |
| ABE8.3-m | | | | | | | | | | | | | | S | | | | |
| ABE8.4-m | | | | | | | | | | H | | | | | | | | |
| ABE8.5-m | | | | | | S | | | | | | | | | | | | |
| ABE8.6-m | | | | | | | | | | | | | | | | | | R |
| ABE8.7-m | | | | | | | | | | | | | | R | | | | |
| ABE8.8-m | | | | | | | | | | H | | R | | R | | | | |
| ABE8.9-m | | | | | Y | | | | | | | R | | R | | | | |
| ABE8.10-m | | | | | | | | | | | | R | | R | | | | R |
| ABE8.11-m | | | | | | | | | | | | T | | R | | | | |
| ABE8.12-m | | | | | | | | | | | | T | | S | | | | |
| ABE8.13-m | | | | | Y | | | | | H | | R | | R | | | | |
| ABE8.14-m | | | | | Y | S | | | | | | | | | | | | |
| ABE8.15-m | | | | | | S | | | | | | R | | | | | | |
| ABE8.16-m | | | | | | S | | | | H | | R | | | | | | |
| ABE8.17-m | | | | | | S | | | | | | | | R | | | | |
| ABE8.18-m | | | | | | S | | | | H | | | | R | | | | |
| ABE8.19-m | | | | | | S | | | | H | | R | | R | | | | |
| ABE8.20-m | | | | | Y | S | | | | H | | R | | R | | | | |
| ABE8.21-m | | | | | | | | | | | | R | | S | | | | |
| ABE8.22-m | | | | | | S | | | | | | | | S | | | | |
| ABE8.23-m | | | | | | S | | | | H | | | | | | | | |
| ABE8.24-m | | | | | | S | | | | H | | T | | | | | | |
| ABE8.1-d | | | | | | | | | | | | T | | | | | | |
| ABE8.2-d | | | | | | | | | | | | R | | | | | | |
| ABE8.3-d | | | | | | | | | | | | | | S | | | | |
| ABE8.4-d | | | | | | | | | | H | | | | | | | | |
| ABE8.5-d | | | | | | S | | | | | | | | | | | | |
| ABE8.6-d | | | | | | | | | | | | | | | | | | R |
| ABE8.7-d | | | | | | | | | | | | | | R | | | | |
| ABE8.8-d | | | | | | | | | | H | | R | | R | | | | |
| ABE8.9-d | | | | | Y | | | | | | | R | | R | | | | |
| ABE8.10-d | | | | | | | | | | | | R | | R | | | | R |
| ABE8.11-d | | | | | | | | | | | | T | | R | | | | |
| ABE8.12-d | | | | | | | | | | | | T | | S | | | | |
| ABE8.13-d | | | | | Y | | | | | H | | R | | R | | | | |
| ABE8.14-d | | | | | Y | S | | | | | | | | | | | | |
| ABE8.15-d | | | | | | S | | | | | | R | | | | | | |
| ABE8.16-d | | | | | | S | | | | H | | R | | | | | | |
| ABE8.17-d | | | | | | S | | | | | | | | R | | | | |
| ABE8.18-d | | | | | | S | | | | H | | | | R | | | | |
| ABE8.19-d | | | | | | S | | | | H | | R | | R | | | | |
| ABE8.20-d | | | | | Y | S | | | | H | | R | | R | | | | |
| ABE8.21-d | | | | | | | | | | | | R | | S | | | | |
| ABE8.22-d | | | | | | S | | | | | | | | S | | | | |
| ABE8.23-d | | | | | | S | | | | H | | | | | | | | |
| ABE8.24-d | | | | | | S | | | | H | | T | | | | | | |

In some embodiments, the base editor is ABE8.1, which comprises or consists essentially of the following sequence or a fragment thereof having adenosine deaminase activity:

ABE8.1_Y147T_CP5_NGC PAM_monomer
(SEQ ID NO: 331)
MSEVEFSHEYWMRHALTLAKRARDEREVPVGA
VLVLNNRVIGEGWNRAIGLHDPTAHAEIMALR
QGGLVMQNYRLIDATLYVTFEPCVMCAGAMIH
SRIGRVVFGVRNAKTGAAGSLMDVLHYPGMNH
RVEITEGILADECAALLCTFFRMPRQVFNAQK
KAQSSTD
SGGSSGGSSGSETPGTSESATPESSGGSSGGS
EIGKATAKYFFYSNIMNFFKTEITLANGEIRK
RPLIETNGETGEIVWDKGRDFATVRKVLSMPQ
VNIVKKTEVQTGGFSKESILPKRNSDKLIARK
KDWDPKKYGGFMQPTVAYSVLVVAKVEKGKSK
KLKSVKELLGITIMERSSFEKNPIDFLEAKGY
KEVKKDLIIKLPKYSLFELENGRKRMLASAKF
LQKGNELALPSKYVNFLYLASHYEKLKGSPED
NEQKQLFVEQHKHYLDEIIEQISEFSKRVILA
DANLDKVLSAYNKHRDKPIREQAENIIHLFTL
TNLGAPRAFKYFDTTIARKEYRSTKEVLDATL
IHQSITGLYETRIDLSQLGGD
GGSGGSGGSGGSGGSGGSGGM
DKKYSIGLAIGTNSVGWAVITDEYKVPSKKFK
VLGNTDRHSIKKNLIGALLFDSGETAEATRLK
RTARRRYTRRKNRICYLQEIFSNEMAKVDDSF
FHRLEESFLVEEDKKHERHPIFGNIVDEVAYH
EKYPTIYHLRKKLVDSTDKADLRLIYLALAHM
IKFRGHFLIEGDLNPDNSDVDKLFIQLVQTYN
QLFEENPINASGVDAKAILSARLSKSRRLENL
IAQLPGEKKNGLFGNLIALSLGLTPNFKSNFD
LAEDAKLQLSKDTYDDDLDNLLAQIGDQYADL
FLAAKNLSDAILLSDILRVNTEITKAPLSASM
IKRYDEHHQDLTLLKALVRQQLPEKYKEIFFD
QSKNGYAGYIDGGASQEEFYKFIKPILEKMDG
TEELLVKLNREDLLRKQRTFDNGSIPHQIHLG
ELHAILRRQEDFYPFLKDNREKIEKILTFRIP
YYVGPLARGNSRFAWMTRKSEETITPWNFEEV
VDKGASAQSFIERMTNFDKNLPNEKVLPKHSL
LYEYFTVYNELTKVKYVTEGMRKPAFLSGEQK
KAIVDLLFKTNRKVTVKQLKEDYFKKIECFDS
VEISGVEDRFNASLGTYHDLLKIIKDKDFLDN
EENEDILEDIVLTLTLFEDREMIEERLKTYAH
LFDDKVMKQLKRRRYTGWGRLSRKLINGIRDK
QSGKTILDFLKSDGFANRNFMQLIHDDSLTFK
EDIQKAQVSGQGDSLHEHIANLAGSPAIKKGI
LQTVKVVDELVKVMGRHKPENIVIEMARENQT
TQKGQKNSRERMKRIEEGIKELGSQILKEHPV
ENTQLQNEKLYLYYLQNGRDMYVDQELDINRL
SDYDVDHIVPQSFLKDDSIDNKVLTRSDKNRG
KSDNVPSEEVVKKMKNYWRQLLNAKLITQRKF
DNLTKAERGGLSELDKAGFIKRQLVETRQITK
HVAQILDSRMNTKYDENDKLIREVKVITLKSK
LVSDFRKDFQFYKVREINNYHHAHDAYLNAVV
GTALIKKYPKLESEFVYGDYKVYDVRKMIAKS
EQEGADKRTADGSEFESPKKKRKV

In the above sequence, the plain text denotes an adenosine deaminase sequence, bold sequence indicates sequence derived from Cas9, the italicized sequence denotes a linker sequence, and the underlined sequence denotes a bipartite nuclear localization sequence. Other ABE8 sequences are provided in the attached sequence listing (SEQ ID NOs: 332-354).

In some embodiments, the base editor is a ninth generation ABE (ABE9). In some embodiments, the ABE9 contains a TadA*9 variant. ABE9 base editors include an adenosine deaminase variant comprising an amino acid sequence, which contains alterations relative to an ABE 7*10 reference sequence, as described herein. Exemplary ABE9 variants are listed in Table 15. Details of ABE9 base editors are described in International PCT Application No. PCT/US2020/049975, which is incorporated herein by reference for its entirety.

TABLE 15
Adenosine Base Editor 9 (ABE9) Variants. In the table, “monomer”
indicates an ABE comprising a single TadA*7.10 comprising the indicated alterations
and “heterodimer” indicates an ABE comprising a TadA*7.10 comprising the indicated
alterations fused to an E. coli TadA adenosine deaminase.

| ABE9 Description | Alterations |
|---|---|
| ABE9.1_monomer | E25F, V82S, Y123H, T133K, Y147R, Q154R |
| ABE9.2_monomer | E25F, V82S, Y123H, Y147R, Q154R |
| ABE9.3_monomer | V82S, Y123H, P124W, Y147R, Q154R |
| ABE9.4_monomer | L51W, V82S, Y123H, C146R, Y147R, Q154R |
| ABE9.5_monomer | P54C, V82S, Y123H, Y147R, Q154R |
| ABE9.6_monomer | Y73S, V82S, Y123H, Y147R, Q154R |
| ABE9.7_monomer | N38G, V82T, Y123H, Y147R, Q154R |
| ABE9.8_monomer | R23H, V82S, Y123H, Y147R, Q154R |
| ABE9.9_monomer | R21N, V82S, Y123H, Y147R, Q154R |
| ABE9.10_monomer | V82S, Y123H, Y147R, Q154R, A158K |
| ABE9.11_monomer | N72K, V82S, Y123H, D139L, Y147R, Q154R, |
| ABE9.12_monomer | E25F, V82S, Y123H, D139M, Y147R, Q154R |
| ABE9.13_monomer | M70V, V82S, M94V, Y123H, Y147R, Q154R |
| ABE9.14_monomer | Q71M, V82S, Y123H, Y147R, Q154R |
| ABE9.15_heterodimer | E25F, V82S, Y123H, T133K, Y147R, Q154R |
| ABE9.16_heterodimer | E25F, V82S, Y123H, Y147R, Q154R |
| ABE9.17_heterodimer | V82S, Y123H, P124W, Y147R, Q154R |
| ABE9.18_heterodimer | L51W, V82S, Y123H, C146R, Y147R, Q154R |
| ABE9.19_heterodimer | P54C, V82S, Y123H, Y147R, Q154R |
| ABE9.2_heterodimer | Y73S, V82S, Y123H, Y147R, Q154R |
| ABE9.21_heterodimer | N38G, V82T, Y123H, Y147R, Q154R |
| ABE9.22_heterodimer | R23H, V82S, Y123H, Y147R, Q154R |
| ABE9.23_heterodimer | R21N, V82S, Y123H, Y147R, Q154R |
| ABE9.24_heterodimer | V82S, Y123H, Y147R, Q154R, A158K |
| ABE9.25_heterodimer | N72K, V82S, Y123H, D139L, Y147R, Q154R, |
| ABE9.26_heterodimer | E25F, V82S, Y123H, D139M, Y147R, Q154R |
| ABE9.27_heterodimer | M70V, V82S, M94V, Y123H, Y147R, Q154R |
| ABE9.28_heterodimer | Q71M, V82S, Y123H, Y147R, Q154R |
| ABE9.29_monomer | E25F_I76Y_V82S_Y123H_Y147R_Q154R |
| ABE9.30_monomer | I76Y_V82T_Y123H_Y147R_Q154R |
| ABE9.31_monomer | N38G_I76Y_V82S_Y123H_Y147R_Q154R |
| ABE9.32_monomer | N38G_I76Y_V82T_Y123H_Y147R_Q154R |
| ABE9.33_monomer | R23H_I76Y V82S_Y123H_Y147R_Q154R |
| ABE9.34_monomer | P54C_I76Y V82S_Y123H_Y147R_Q154R |
| ABE9.35_monomer | R21N_I76Y V82S_Y123H_Y147R_Q154R |
| ABE9.36_monomer | I76Y_V82S Y123H_D138M_Y147R_Q154R |
| ABE9.37_monomer | Y72S_I76Y_V82S_Y123H_Y147R_Q154R |
| ABE9.38_heterodimer | E25F_I76Y_V82S_Y123H_Y147R_Q154R |
| ABE9.39_heterodimer | I76Y_V82T Y123H Y147R_Q154R |
| ABE9.40_heterodimer | N38G_I76Y_V82S_Y123H_Y147R_Q154R |
| ABE9.41_heterodimer | N38G_I76Y_V82T_Y123H_Y147R_Q154R |
| ABE9.42_heterodimer | R23H_I76Y_V82S_Y123H_Y147R_Q154R |
| ABE9.43_heterodimer | P54C_I76Y_V82S_Y123H_Y147R_Q154R |
| ABE9.44_heterodimer | R21N_I76Y_V82S_Y123H_Y147R_Q154R |
| ABE9.45_heterodimer | I76Y_V82S_Y123H_D138M_Y147R_Q154R |
| ABE9.46_heterodimer | Y72S_I76Y_V82S_Y123H_Y147R_Q154R |
| ABE9.47_monomer | N72K_V82S, Y123H, Y147R, Q154R |
| ABE9.48_monomer | Q71M_V82S, Y123H, Y147R, Q154R |
| ABE9.49_monomer | M70V, V82S, M94V, Y123H, Y147R, Q154R |
| ABE9.50_monomer | V82S, Y123H, T133K, Y147R, Q154R |
| ABE9.51_monomer | V82S, Y123H, T133K, Y147R, Q154R, |
| | A158K |
| ABE9.52_monomer | M70V, Q71M, N72K, V82S, Y123H, Y147R, |
| | Q154R |
| ABE9.53_heterodimer | N72K_V82S, Y123H, Y147R, Q154R |
| ABE9.54_heterodimer | Q71M_V82S, Y123H, Y147R, Q154R |
| ABE9.55_heterodimer | M70V, V82S, M94V, Y123H, Y147R, Q154R |
| ABE9.56_heterodimer | V82S, Y123H, T133K, Y147R, Q154R |
| ABE9.57_heterodimer | V82S, Y123H, T133K, Y147R, Q154R, |
| | A158K |
| ABE9.58_heterodimer | M70V, Q71M, N72K, V82S, Y123H, Y147R, |
| | Q154R |

In some embodiments, the base editor includes an adenosine deaminase variant comprising an amino acid sequence, which contains alterations relative to an ABE 7*10 reference sequence, as described herein. The term “monomer” as used in Table 15.1 refers to a monomeric form of TadA*7.10 comprising the alterations described. The term “heterodimer” as used in Table 15.1 refers to the specified wild-type E. coli TadA adenosine deaminase fused to a TadA*7.10 comprising the alterations as described.

TABLE 15.1
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + Q154S + |
| | | N157K |
| ABE-828m | MSP828 | monomer_TadA*7.10 + I76Y + V82G + Y147D + F149Y + Q154S + |
| | | D167N |
| ABE-829m | MSP829 | monomer_TadA*7.10 + L36H + I76Y + V82G + Y147D + F149Y + |
| | | Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + Y147T + |
| | | Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + Y147D + |
| | | F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain. In some embodiments, the base editor comprises a domain comprising all or a portion of a nucleic acid polymerase. In some embodiments, a base editor can comprise as a domain all or a portion (e.g., a functional portion) of a nucleic acid polymerase (NAP). For example, a base editor comprises all or a portion (e.g., a functional portion) of a eukaryotic NAP. In some embodiments, a NAP or portion (e.g., a functional portion) thereof incorporated into a base editor is a DNA polymerase. In some embodiments, a NAP or portion thereof incorporated into a base editor has translesion polymerase activity. In some embodiments, a NAP or portion thereof incorporated into a base editor is a translesion DNA polymerase. In some embodiments, a NAP or portion thereof incorporated into a base editor is a Rev7, Rev1 complex, polymerase iota, polymerase kappa, or polymerase eta. In some embodiments, a NAP or portion thereof incorporated into a base editor is a eukaryotic polymerase alpha, beta, gamma, delta, epsilon, gamma, eta, iota, kappa, lambda, mu, or nu component. In some embodiments, a NAP or portion thereof incorporated into a base editor comprises an amino acid sequence that is at least 75%, 80%, 85%, 90%, 95%, 96%, 97%, 98%, 99%, or 99.5% identical to a nucleic acid polymerase (e.g., a translesion DNA polymerase). In some embodiments, a nucleic acid polymerase or portion thereof incorporated into a base editor is a translesion DNA polymerase.

In some embodiments, a domain of the base editor comprises multiple domains. For example, the base editor comprising a polynucleotide programmable nucleotide binding domain derived from Cas9 can comprise a REC lobe and an NUC lobe corresponding to the REC lobe and NUC lobe of a wild-type or natural Cas9. In another example, the base editor can comprise one or more of a RuvCI domain, BH domain, REC1 domain, REC2 domain, RuvCII domain, L1 domain, HNH domain, L2 domain, RuvCIII domain, WED domain, TOPO domain or CTD domain. In some embodiments, one or more domains of the base editor comprise a mutation (e.g., substitution, insertion, deletion) relative to a wild-type version of a polypeptide comprising the domain. For example, an HNH domain of a polynucleotide programmable DNA binding domain can comprise an H840A substitution. In another example, a RuvCI domain of a polynucleotide programmable DNA binding domain can comprise a D10A substitution.

Different domains (e.g., adjacent domains) of the base editor disclosed herein can be connected to each other with or without the use of one or more linker domains (e.g., an XTEN linker domain). In some embodiments, a linker domain can be a bond (e.g., covalent bond), chemical group, or a molecule linking two molecules or moieties, e.g., two domains of a fusion protein, such as, for example, a first domain (e.g., Cas9-derived domain) and a second domain (e.g., an adenosine deaminase domain or a cytidine deaminase domain). In some embodiments, a linker is a covalent bond (e.g., a carbon-carbon bond, disulfide bond, carbon-hetero atom bond, etc.). In certain embodiments, a linker is a carbon nitrogen bond of an amide linkage. In certain embodiments, a linker is a cyclic or acyclic, substituted or unsubstituted, branched or unbranched aliphatic or heteroaliphatic linker. In certain embodiments, a linker is polymeric (e.g., polyethylene, polyethylene glycol, polyamide, polyester, etc.). In certain embodiments, a linker comprises a monomer, dimer, or polymer of aminoalkanoic acid. In some embodiments, a linker comprises an aminoalkanoic acid (e.g. glycine, ethanoic acid, alanine, beta-alanine, 3-aminopropanoic acid, 4-aminobutanoic acid, 5-pentanoic acid, etc.). In some embodiments, a linker comprises a monomer, dimer, or polymer of aminohexanoic acid (Ahx). In certain embodiments, a linker is based on a carbocyclic moiety (e.g., cyclopentane, cyclohexane). In other embodiments, a linker comprises a polyethylene glycol moiety (PEG). In certain embodiments, a linker comprises an aryl or heteroaryl moiety. In certain embodiments, the linker is based on a phenyl ring. A linker can include functionalized moieties to facilitate attachment of a nucleophile (e.g., thiol, amino) from the peptide to the linker. Any electrophile can be used as part of the linker. Exemplary electrophiles include, but are not limited to, activated esters, activated amides, Michael acceptors, alkyl halides, aryl halides, acyl halides, and isothiocyanates. In some embodiments, a linker joins a gRNA binding domain of an RNA-programmable nuclease, including a Cas9 nuclease domain, and the catalytic domain of a nucleic acid editing protein. In some embodiments, a linker joins a dCas9 and a second domain (e.g., UGI, etc.).

### Linkers

In certain embodiments, linkers may be used to link any of the peptides or peptide domains of the present disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Typically, a linker is positioned between, or flanked by, two groups, molecules, or other moieties and connected to each one via a covalent bond, thus connecting the two. In some embodiments, a linker is an amino acid or a plurality of amino acids (e.g., a peptide or protein). In some embodiments, a linker is an organic molecule, group, polymer, or chemical moiety. In some embodiments, a linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is about 3 to about 104 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100) amino acids in length. Longer or shorter linkers are also contemplated.

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)ₙ (SEQ ID NO: 246), (GGGGS)ₙ (SEQ ID NO: 247), and (G)ₙ to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)ₙ) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)ₙ motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGSP
TSTEEGTSTEPSEGSAPGTSTEPSEGSAPGTSESATPESGPGSEPATSGG
SGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS. In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence:

(SEQ ID NO: 360)
SGGSSGGSSGSETPGTSESATPESSGGSSGGSSGGSSGGS.

In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEEG
TSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P (AP) 4 (SEQ ID NO: 367), P (AP) 7 (SEQ ID NO: 368), P (AP) 10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.

In another embodiment, the base editor system comprises a component (protein) that interacts non-covalently with a deaminase (DNA deaminase), e.g., an adenosine or a cytidine deaminase, and transiently attracts the adenosine or cytidine deaminase to the target nucleobase in a target polynucleotide sequence for specific editing, with minimal or reduced bystander or target-adjacent effects. Such anon-covalent system and method involving deaminase-interacting proteins serves to attract a DNA deaminase to a particular genomic target nucleobase and decouples the events of on-target and target-adjacent editing, thus enhancing the achievement of more precise single base substitution mutations. In an embodiment, the deaminase-interacting protein binds to the deaminase (e.g., adenosine deaminase or cytidine deaminase) without blocking or interfering with the active (catalytic) site of the deaminase from engaging the target nucleobase (e.g., adenosine or cytidine, respectively). Such as system, termed “MagnEdit,” involves interacting proteins tethered to a Cas9 and gRNA complex and can attract a co-expressed adenosine or cytidine deaminase (either exogenous or endogenous) to edit a specific genomic target site, and is described in McCann, J. et al., 2020, “MagnEdit—interacting factors that recruit DNA-editing enzymes to single base targets,” Life-Science-Alliance, Vol. 3, No. 4 (e201900606), (doi 10.26508/Isa.201900606), the contents of which are incorporated by reference herein in their entirety. In an embodiment, the DNA deaminase is an adenosine deaminase variant (e.g., TadA*8) as described herein.

In another embodiment, a system called “Suntag,” involves non-covalently interacting components used for recruiting protein (e.g., adenosine deaminase or cytidine deaminase) components, or multiple copies thereof, of base editors to polynucleotide target sites to achieve base editing at the site with reduced adjacent target editing, for example, as described in Tanenbaum, M. E. et al., “A protein tagging system for signal amplification in gene expression and fluorescence imaging,” Cell. 2014 October 23; 159(3): 635-646. doi:10.1016/j.cell.2014.09.039; and in Huang, Y.-H. et al., 2017, “DNA epigenome editing using CRISPR-Cas SunTag-directed DNMT3A,” Genome Biol 18: 176. doi:10.1186/s13059-017-1306-z, the contents of each of which are incorporated by reference herein in their entirety. In an embodiment, the DNA deaminase is an adenosine deaminase variant (e.g., TadA*8) as described herein.

Nucleic Acid Programmable DNA Binding Proteins with Guide RNAs

Provided herein are compositions and methods for base editing in cells (e.g., immune cells (e.g., T- or NK-cells). Further provided herein are compositions comprising a guide polynucleic acid sequence, e.g. a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g. a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in an immune cell (e.g., T- or NK-cell) through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is an RNA sequence. In some embodiments, the target sequence is a sequence in the genome of a mammal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 6 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence. In some embodiments, the 3′ end of the target sequence is immediately adjacent to an e.g., TTN, DTTN, GTTN, ATTN, ATTC, DTTNT, WTTN, HATY, TTTN, TTTV, TTTC, TG, RTR, or YTN PAM site.

It will be understood that the numbering of the specific positions or residues in the respective sequences depends on the particular protein and numbering scheme used. Numbering might differ, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins or complexes disclosed herein, to a target site, e.g., a site comprising a mutation to be edited, it is typically necessary to co-express the fusion protein or complex together with a guide RNA. As explained in more detail elsewhere herein, a guide RNA typically comprises a tracrRNA framework allowing for napDNAbp (e.g., Cas9 or Cas12) binding, and a guide sequence, which confers sequence specificity to the napDNAbp:nucleic acid editing enzyme/domain fusion protein or complex. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting napDNAbp:nucleic acid editing enzyme/domain fusion proteins or complexes to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins or complexes to specific target sequences are provided herein.

Distinct portions of sgRNA are predicted to form various features that interact with Cas9 (e.g., SpyCas9) and/or the DNA target. Six conserved modules have been identified within native crRNA:tracrRNA duplexes and single guide RNAs (sgRNAs) that direct Cas9 endonuclease activity (see Briner et al., Guide RNA Functional Modules Direct Cas9 Activity and Orthogonality Mol Cell. 2014 Oct. 23; 56(2):333-339). The six modules include the spacer responsible for DNA targeting, the upper stem, bulge, lower stem formed by the CRISPR repeat:tracrRNA duplex, the nexus, and hairpins from the 3′ end of the tracrRNA. The upper and lower stems interact with Cas9 mainly through sequence-independent interactions with the phosphate backbone. In some embodiments, the upper stem is dispensable. In some embodiments, the conserved uracil nucleotide sequence at the base of the lower stem is dispensable. The bulge participates in specific side-chain interactions with the Rec domain of Cas9. The nucleobase of U44 interacts with the side chains of Tyr 325 and His 328, while G43 interacts with Tyr 329. The nexus forms the core of the sgRNA:Cas9 interactions and lies at the intersection between the sgRNA and both Cas9 and the target DNA. The nucleobases of A51 and A52 interact with the side chain of Phe 1105; U56 interacts with Arg 457 and Asn 459; the nucleobase of U59 inserts into a hydrophobic pocket defined by side chains of Arg 74, Asn 77, Pro 475, Leu 455, Phe 446, and Ile 448; C60 interacts with Leu 455, Ala 456, and Asn 459, and C61 interacts with the side chain of Arg 70, which in turn interacts with C15. In some embodiments, one or more of these mutations are made in the bulge and/or the nexus of a sgRNA for a Cas9 (e.g., spyCas9) to optimize sgRNA:Cas9 interactions.

Moreover, the tracrRNA nexus and hairpins are critical for Cas9 pairing and can be swapped to cross orthogonality barriers separating disparate Cas9 proteins, which is instrumental for further harnessing of orthogonal Cas9 proteins. In some embodiments, the nexus and hairpins are swapped to target orthogonal Cas9 proteins. In some embodiments, a sgRNA is dispensed of the upper stem, hairpin 1, and/or the sequence flexibility of the lower stem to design a guide RNA that is more compact and conformationally stable. In some embodiments, the modules are modified to optimize multiplex editing using a single Cas9 with various chimeric guides or by concurrently using orthogonal systems with different combinations of chimeric sgRNAs. Details regarding guide functional modules and methods thereof are described, for example, in Briner et al., Guide RNA Functional Modules Direct Cas9 Activity and Orthogonality Mol Cell. 2014 Oct. 23; 56(2):333-339, the contents of which is incorporated by reference herein in its entirety.

The domains of the base editor disclosed herein can be arranged in any order. Non-limiting examples of a base editor comprising a fusion protein comprising e.g., a polynucleotide-programmable nucleotide-binding domain (e.g., Cas9 or Cas12) and a deaminase domain (e.g., cytidine or adenosine deaminase) can be arranged as follows:
NH2-[nucleobase editing domain]-Linker1-[nucleobase editing domain]-COOH;
NH2-[deaminase]-Linker1-[nucleobase editing domain]-COOH;
NH2-[deaminase]-Linker1-[nucleobase editing domain]-Linker2-[UGI]—COOH;
NH2-[deaminase]-Linker1-[nucleobase editing domain]-COOH;
NH2-[adenosine deaminase]-Linker1-[nucleobase editing domain]-COOH;
NH2-[nucleobase editing domain]-[deaminase]-COOH;
NH2-[deaminase]-[nucleobase editing domain]-[inosine BER inhibitor]-COOH;
NH2-[deaminase]-[inosine BER inhibitor]-[nucleobase editing domain]-COOH;
NH2-[inosine BER inhibitor]-[deaminase]-[nucleobase editing domain]-COOH;
NH2-[nucleobase editing domain]-[deaminase]-[inosine BER inhibitor]-COOH;
NH2-[nucleobase editing domain]-[inosine BER inhibitor]-[deaminase]-COOH;
NH2-[inosine BER inhibitor]-[nucleobase editing domain]-[deaminase]-COOH;
NH2-[nucleobase editing domain]-Linker1-[deaminase]-Linker2-[nucleobase editing domain]-COOH;
NH2-[nucleobase editing domain]-Linker1-[deaminase]-[nucleobase editing domain]-COOH;
NH2-[nucleobase editing domain]-[deaminase]-Linker2-[nucleobase editing domain]-COOH;
NH2-[nucleobase editing domain]-[deaminase]-[nucleobase editing domain]-COOH;
NH2-[nucleobase editing domain]-Linker1-[deaminase]-Linker2-[nucleobase editing domain]-[inosine BER inhibitor]-COOH;
NH2-[nucleobase editing domain]-Linker1-[deaminase]-[nucleobase editing domain]-[inosine BER inhibitor]-COOH;
NH2-[nucleobase editing domain]-[deaminase]-Linker2-[nucleobase editing domain]-[inosine BER inhibitor]-COOH;
NH2-[nucleobase editing domain]-[deaminase]-[nucleobase editing domain]-[inosine BER inhibitor]-COOH;
NH2-[inosine BER inhibitor]-[nucleobase editing domain]-Linker1-[deaminase]-Linker2-[nucleobase editing domain]-COOH;
NH2-[inosine BER inhibitor]-[nucleobase editing domain]-Linker1-[deaminase]-[nucleobase editing domain]-COOH;
NH2-[inosine BER inhibitor]-[nucleobase editing domain]-[deaminase]-Linker2-[nucleobase editing domain]-COOH; or
NH2-[inosine BER inhibitor]NH2-[nucleobase editing domain]-[deaminase]-[nucleobase editing domain]-COOH.

In some embodiments, the base editing fusion proteins or complexes provided herein need to be positioned at a precise location, for example, where a target base is placed within a defined region (e.g., a “deamination window”). In some embodiments, a target can be within a 4-base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a napDNAbp domain. In some embodiments, an NLS of the base editor is localized C-terminal to a napDNAbp domain.

Non-limiting examples of protein domains which can be included in the fusion protein or complexes include a deaminase domain (e.g., adenosine deaminase or cytidine deaminase), a uracil glycosylase inhibitor (UGI) domain, epitope tags, reporter gene sequences, and/or protein domains having one or more of the activities described herein.

A domain may be detected or labeled with an epitope tag, a reporter protein, other binding domains. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the present disclosure is used for editing a target gene or polynucleotide sequence of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins or complexes comprising a Cas9 domain and a cytidine or adenosine deaminase, as disclosed herein, to a target site, e.g., a site comprising a mutation to be edited, it is typically necessary to co-express the fusion protein or complex together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein or complex. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins or complexes to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins or complexes to specific target sequences are provided herein.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations. In some embodiments, an intended mutation is a mutation that is generated by a specific base editor (e.g., adenosine base editor or cytidine base editor) bound to a guide polynucleotide (e.g., gRNA), specifically designed to generate the intended mutation. In some embodiments, the intended mutation is in a gene associated with a target antigen associated with a disease or disorder. In some embodiments, the intended mutation is an adenine (A) to guanine (G) point mutation (e.g., SNP) in a gene associated with a target antigen associated with a disease or disorder. In some embodiments, the intended mutation is an adenine (A) to guanine (G) point mutation within the coding region or non-coding region of a gene (e.g., regulatory region or element). In some embodiments, the intended mutation is a cytosine (C) to thymine (T) point mutation (e.g., SNP) in a gene associated with a target antigen associated with a disease or disorder. In some embodiments, the intended mutation is a cytosine (C) to thymine (T) point mutation within the coding region or non-coding region of a gene (e.g., regulatory region or element). In some embodiments, the intended mutation is a point mutation that generates a STOP codon, for example, a premature STOP codon within the coding region of a gene. In some embodiments, the intended mutation is a mutation that eliminates a stop codon.

The base editors of the present disclosure advantageously modify a specific nucleotide base encoding a protein without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate) a specific nucleotide within a nucleic acid, without generating a large number of insertions or deletions (i.e., indels) in the nucleic acid. In some embodiments, it is desirable to generate base editors that efficiently modify (e.g. mutate or methylate) a specific nucleotide within a nucleic acid, without generating a large number of insertions or deletions (i.e., indels) in the nucleic acid. In certain embodiments, any of the base editors provided herein can generate a greater proportion of intended modifications (e.g., methylations) versus indels. In certain embodiments, any of the base editors provided herein can generate a greater proportion of intended modifications (e.g., mutations) versus indels.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation in a nucleic acid (e.g. a nucleic acid within a genome of a subject) without generating a considerable number of unintended mutations (e.g., spurious off-target editing or bystander editing). In some embodiments, an intended mutation is a mutation that is generated by a specific base editor bound to a gRNA, specifically designed to generate the intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g. intended mutations:unintended mutations) that is greater than 1:1. In some embodiments, any of the base editors provided herein are capable of generating a ratio of intended mutations to unintended mutations that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described herein may be applied to any of the fusion proteins or complexes, or methods of using the fusion proteins or complexes provided herein.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence, and may affect the gene product. In essence therefore, the gene editing modification described herein may result in a modification of the gene, structurally and/or functionally, wherein the expression of the gene product may be modified, for example, the expression of the gene is knocked out; or conversely, enhanced, or, in some circumstances, the gene function or activity may be modified. Using the methods disclosed herein, a base editing efficiency may be determined as the knockdown efficiency of the gene in which the base editing is performed, wherein the base editing is intended to knockdown the expression of the gene. A knockdown level may be validated quantitatively by determining the expression level by any detection assay, such as assay for protein expression level, for example, by flow cytometry; assay for detecting RNA expression such as quantitative RT-PCR, northern blot analysis, or any other suitable assay such as pyrosequencing; and may be validated qualitatively by nucleotide sequencing reactions.

In some embodiments, the modification, e.g., single base edit results in at least 10% reduction of the gene targeted expression. In some embodiments, the base editing efficiency may result in at least 10% reduction of the gene targeted expression. In some embodiments, the base editing efficiency may result in at least 20% reduction of the gene targeted expression. In some embodiments, the base editing efficiency may result in at least 30% reduction of the gene targeted expression. In some embodiments, the base editing efficiency may result in at least 40% reduction of the gene targeted expression. In some embodiments, the base editing efficiency may result in at least 50% reduction of the gene targeted expression. In some embodiments, the base editing efficiency may result in at least 60% reduction of the targeted gene expression. In some embodiments, the base editing efficiency may result in at least 70% reduction of the targeted gene expression. In some embodiments, the base editing efficiency may result in at least 80% reduction of the targeted gene expression. In some embodiments, the base editing efficiency may result in at least 90% reduction of the targeted gene expression. In some embodiments, the base editing efficiency may result in at least 91% reduction of the targeted gene expression. In some embodiments, the base editing efficiency may result in at least 92% reduction of the targeted gene expression. In some embodiments, the base editing efficiency may result in at least 93% reduction of the targeted gene expression. In some embodiments, the base editing efficiency may result in at least 94% reduction of the targeted gene expression. In some embodiments, the base editing efficiency may result in at least 95% reduction of the targeted gene expression. In some embodiments, the base editing efficiency may result in at least 96% reduction of the targeted gene expression. In some embodiments, the base editing efficiency may result in at least 97% reduction of the targeted gene expression. In some embodiments, the base editing efficiency may result in at least 98% reduction of the targeted gene expression. In some embodiments, the base editing efficiency may result in at least 99% reduction of the targeted gene expression. In some embodiments, the base editing efficiency may result in knock-out (100% knockdown of the gene expression) of the gene that is targeted.

In some embodiments, any of the base editor systems provided herein result in less than 50%, less than 40%, less than 30%, less than 20%, less than 19%, less than 18%, less than 17%, less than 16%, less than 15%, less than 14%, less than 13%, less than 12%, less than 110, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

In some embodiments, targeted modifications, e.g., single base editing, are used simultaneously to target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 different endogenous sequences for base editing with different guide RNAs. In some embodiments, targeted modifications, e.g. single base editing, are used to sequentially target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, or more different endogenous gene sequences for base editing with different guide RNAs.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations (i.e., mutation of bystanders). In some embodiments, any of the base editors provided herein are capable of generating at least 0.01% of intended mutations (i.e., at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, any of the base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence. In some embodiments, any of the base editor systems comprising one of the ABE8 base editor variants described herein result in less than 0.8% indel formation in the target polynucleotide sequence. In some embodiments, any of the base editor systems comprising one of the ABE8 base editor variants described herein result in at most 0.8% indel formation in the target polynucleotide sequence. In some embodiments, any of the base editor systems comprising one of the ABE8 base editor variants described herein result in less than 0.3% indel formation in the target polynucleotide sequence. In some embodiments, any of the base editor systems comprising one of the ABE8 base editor variants described results in lower indel formation in the target polynucleotide sequence compared to a base editor system comprising one of ABE7 base editors. In some embodiments, any of the base editor systems comprising one of the ABE8 base editor variants described herein results in lower indel formation in the target polynucleotide sequence compared to a base editor system comprising an ABE7.10.

In some embodiments, any of the base editor systems comprising one of the ABE8 base editor variants described herein has reduction in indel frequency compared to a base editor system comprising one of the ABE7 base editors. In some embodiments, any of the base editor systems comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising one of the ABE7 base editors. In some embodiments, a base editor system comprising one of the ABE8 base editor variants described herein has at least 0.010%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising an ABE7.10.

The present disclosure provides adenosine deaminase variants (e.g., ABE8 variants) that have increased efficiency and specificity. In particular, the adenosine deaminase variants described herein are more likely to edit a desired base within a polynucleotide, and are less likely to edit bases that are not intended to be altered (e.g., “bystanders”).

In some embodiments, any of the base editor systems provided herein result in less than 70%, less than 65%, less than 60%, less than 55%, 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% bystander editing of one or more nucleotides (e.g., an off-target nucleotide).

In some embodiments, any of the base editing system comprising one of the ABE8 base editor variants described herein has reduced bystander editing or mutations. In some embodiments, an unintended editing or mutation is a bystander mutation or bystander editing, for example, base editing of a target base (e.g., A or C) in an unintended or non-target position in a target window of a target nucleotide sequence. In some embodiments, any of the base editing system comprising one of the ABE8 base editor variants described herein has reduced bystander editing or mutations compared to a base editor system comprising an ABE7 base editor, e.g., ABE7.10. In some embodiments, any of the base editing system comprising one of the ABE8 base editor variants described herein has reduced bystander editing or mutations by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced bystander editing or mutations by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the base editing system comprising one of the ABE8 base editor variants described herein has reduced spurious editing. In some embodiments, an unintended editing or mutation is a spurious mutation or spurious editing, for example, non-specific editing or guide independent editing of a target base (e.g., A or C) in an unintended or non-target region of the genome. In some embodiments, any of the base editing system comprising one of the ABE8 base editor variants described herein has reduced spurious editing compared to a base editor system comprising an ABE7 base editor, e.g., ABE7.10. In some embodiments, any of the base editing system comprising one of the ABE8 base editor variants described herein has reduced spurious editing by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced spurious editing by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% base editing efficiency. In some embodiments, the base editing efficiency may be measured by calculating the percentage of edited nucleobases in a population of cells. In some embodiments, any of the ABE8 base editor variants described herein have base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases in a population of cells.

In some embodiments, any of the ABE8 base editor variants described herein has higher base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher base editing efficiency compared to an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% on-target base editing efficiency. In some embodiments, any of the ABE8 base editor variants described herein have on-target base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited target nucleobases in a population of cells.

In some embodiments, any of the ABE8 base editor variants described herein has higher on-target base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher on-target base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA. In some embodiments, an ABE8 base editor delivered via a nucleic acid based delivery system, e.g., an mRNA, has on-target editing efficiency of at least at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases. In some embodiments, an ABE8 base editor delivered by an mRNA system has higher base editing efficiency compared to an ABE8 base editor delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% on-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target editing efficiency when delivered by an mRNA system compared to when delivered by a plasmid or vector system.

In some embodiments, any of the base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in the target polynucleotide sequence.

In some embodiments, any of the ABE8 base editor variants described herein has lower guided off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guided off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold lower guided off-target editing efficiency when delivered by an mRNA system compared to when delivered by a plasmid or vector system. In some embodiments, any of the ABE8 base editor variants described herein has at least about 2.2 fold decrease in guided off-target editing efficiency when delivered by an mRNA system compared to when delivered by a plasmid or vector system.

In some embodiments, any of the ABE8 base editor variants described herein has lower guide-independent off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guide-independent off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 5.0 fold, at least 10.0 fold, at least 20.0 fold, at least 50.0 fold, at least 70.0 fold, at least 100.0 fold, at least 120.0 fold, at least 130.0 fold, or at least 150.0 fold lower guide-independent off-target editing efficiency when delivered by an mRNA system compared to when delivered by a plasmid or vector system. In some embodiments, ABE8 base editor variants described herein has 134.0 fold decrease in guide-independent off-target editing efficiency (e.g., spurious RNA deamination) when delivered by an mRNA system compared to when delivered by a plasmid or vector system. In some embodiments, ABE8 base editor variants described herein does not increase guide-independent mutation rates across the genome.

In some embodiments, a single gene delivery event (e.g., by transduction, transfection, electroporation or any other method) can be used to target base editing of 5 sequences within a cell's genome. In some embodiments, a single gene delivery event can be used to target base editing of 6 sequences within a cell's genome. In some embodiments, a single gene delivery event can be used to target base editing of 7 sequences within a cell's genome. In some embodiments, a single electroporation event can be used to target base editing of 8 sequences within a cell's genome. In some embodiments, a single gene delivery event can be used to target base editing of 9 sequences within a cell's genome. In some embodiments, a single gene delivery event can be used to target base editing of 10 sequences within a cell's genome. In some embodiments, a single gene delivery event can be used to target base editing of 20 sequences within a cell's genome. In some embodiments, a single gene delivery event can be used to target base editing of 30 sequences within a cell's genome. In some embodiments, a single gene delivery event can be used to target base editing of 40 sequences within a cell's genome. In some embodiments, a single gene delivery event can be used to target base editing of 50 sequences within a cell's genome.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in at least 50% of a cell population that have been successfully edited (i.e., cells that have been successfully engineered). In some embodiments, the base editing method described herein results in at least 55% of a cell population that have been successfully edited. In some embodiments, the base editing method described herein results in at least 60% of a cell population that have been successfully edited. In some embodiments, the base editing method described herein results in at least 65% of a cell population that have been successfully edited. In some embodiments, the base editing method described herein results in at least 70% of a cell population that have been successfully edited. In some embodiments, the base editing method described herein results in at least 75% of a cell population that have been successfully edited. In some embodiments, the base editing method described herein results in at least 80% of a cell population that have been successfully edited. In some embodiments, the base editing method described herein results in at least 85% of a cell population that have been successfully edited. In some embodiments, the base editing method described herein results in at least 90% of a cell population that have been successfully edited. In some embodiments, the base editing method described herein results in at least 95% of a cell population that have been successfully edited. In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event. In some embodiments, the engineered cell population can be further expanded in vitro by about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins or complexes, or methods of using the fusion proteins or complexes provided herein.

Details of base editor efficiency are described in International PCT Application Nos. PCT/US2017/045381 (WO 2018/027078) and PCT/US2016/058344 (WO 2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference. In some embodiments, editing of a plurality of nucleobase pairs in one or more genes using the methods provided herein results in formation of at least one intended mutation. In some embodiments, said formation of said at least one intended mutation results in the disruption the normal function of a gene. In some embodiments, said formation of said at least one intended mutation results decreases or eliminates the expression of a protein encoded by a gene. It should be appreciated that multiplex editing can be accomplished using any method or combination of methods provided herein.

### Multiplex Editing

In some embodiments, the base editor system provided herein is capable of multiplex editing of a plurality of nucleobase pairs in one or more PLC genes (e.g., β2M, TAP1, TAP2, Tapasin) and/or CD58, and/or regulatory elements thereof, in an immune cell (e.g., T- or NK-cell). In some embodiments, the base editor system provided herein is capable of multiplex editing of a plurality of nucleobase pairs in one or more additional gene sequences or regulatory element thereof (e.g., TRAC, CD58, CIITA) in an immune cell (e.g., T- or NK-cell). In some embodiments, the additional gene sequences or regulatory elements are selected from TCRα Chain (TRAC), Cluster of Differentiation 58 (CD58), and Class II, Major Histocompatibility Complex Transactivator (CIITA). In some embodiments, the base editor system provided herein is capable of multiplex editing of a plurality of nucleobase pairs in one or more of β2M, TAP1, TAP2, and/or Tapasin encoding genes in combination with one or more of TRAC, CD58, and/or CIITA encoding genes in an immune cell (e.g., T- or NK-cell). In some embodiments, the base editor system provided herein is capable of multiplex editing of a plurality of nucleobase pairs in one or more of Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), HLA-A, HLA-B, HLA-C, MHC class I polypeptide-related sequence A (MICA), MHC class I polypeptide-related sequence B (MICB) polypeptide, nectin cell adhesion molecule 2 (Nectin-2), UL16 binding protein 1-6 (ULBP), beta-2 microglobulin, CD48, CD58, Protein Disulfide Isomerase Family A Member 3 (PDIA3/ERp57), T Cell Receptor Alpha Constant polypeptides, HLA-A, HLA-B, HLA-C, TCRα Chain (TRAC), and Class II, Major Histocompatibility Complex Transactivator (CIITA) encoding genes. In some instances, the methods of the present disclosure involve knocking out expression of the following genes in an allogeneic immune cell, or a precursor thereof. CD5, B2M, CD3 gamma, CD3 epsilon, CIITA, and PD-1 (PD1).

In some instances, the methods of the present disclosure involve knocking out HLA-A, HLA-B, and CIITA expression in a cell. In some cases, the methods further involve knocking out expression of one or more of CD155, Nectin-2, CD48, MICA, MICB, and ULBP in the cell. The disclosure further provides cells modified according to the methods provided herein.

In some cases, the methods of the present disclosure involve knocking out an HLA-I peptide presentation pathway in a cell. In various instances, knocking out the HLA-I peptide presentation pathway involves knocking out expression of one or more genes listed herein (e.g., B2M, TAP1, etc.).

In some embodiments, cells with HLA-A and HLA-B expression knocked-out but with HLA-C expression not knocked out are prepared by sorting (e.g., using flow cytometry) a population of cells contacted with a base editing system (e.g., a base editing system containing the guide TSBTx4193 and/or TSBTx4194) of the disclosure for those cells surface-expressing HLA-C but not surface-expressing HLA-A or HLA-B. In some embodiments, cells with HLA-A and HLA-B expression knocked-out but with HLA-C expression not knocked out are prepared by identifying a donor having an HLA-C allele that is not knocked-out by a gRNA that knocks-out HLA-A and HLA-B expression (e.g., said HLA-C allele does not share a corresponding PAM sequence with the PAM sequence adjacent to the gRNA spacer sequence in the HLA-A and HLA-B alleles), and then contacting a population of cells from the donor with a base editing system of the disclosure comprising said gRNA.

In embodiments, the methods of the invention involve introducing one or more nucleobase alterations to a polynucleotide encoding an HLA-A polypeptide and to a polynucleotide encoding an HLA-B polypeptide. In embodiments, the methods of the invention involve introducing one or more nucleobase alterations to a polynucleotide encoding an HLA-A polypeptide and to a polynucleotide encoding an HLA-C polypeptide. In embodiments, the methods of the invention involve introducing one or more nucleobase alterations to a polynucleotide encoding an HLA-B polypeptide and to a polynucleotide encoding an HLA-C polypeptide. In embodiments, the alterations result in reduced or eliminated expression of the polypeptide (i.e., HLA-A, HLA-B, and/or HLA-C). In some cases, the methods of the invention involve knocking out or reducing expression of HLA-A and HLA-B expression in a cell, knocking out or reducing expression of HLA-A and HLA-C expression in a cell, and/or knocking out or reducing HLA-A and HLA-C expression in a cell. In various aspects, the invention of the disclosure features a cell comprising any one or more of the nucleobase alterations produced by the methods provided herein.

In some embodiments, the plurality of nucleobase pairs is located in the same gene or in one or more genes or polynucleotide sequences, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. In some embodiments, the multiplex editing comprises at least one guide polynucleotide that does or does not require a PAM sequence to target binding to a target polynucleotide sequence. In some embodiments, the multiplex editing comprises a mix of at least one guide polynucleotide that does not require a PAM sequence to target binding to a target polynucleotide sequence and at least one guide polynucleotide that require a PAM sequence to target binding to a target polynucleotide sequence. It should be appreciated that the characteristics of the multiplex editing using any of the base editors as described herein can be applied to any combination of methods using any base editor provided herein. It should also be appreciated that the multiplex editing using any of the base editors as described herein comprises a sequential editing of a plurality of nucleobase pairs.

In some embodiments, the plurality of nucleobase pairs are in one more genes or polynucleotide sequences. In some embodiments, the plurality of nucleobase pairs is in the same gene or polynucleotide sequence. In some embodiments, at least one gene in the one more genes is located in a different locus.

In some embodiments, the editing is editing of the plurality of nucleobase pairs in at least one protein coding region, in at least one protein non-coding region, or in at least one protein coding region and at least one protein non-coding region.

In some embodiments, the editing is in conjunction with one or more guide polynucleotides. In some embodiments, the base editor system comprises one or more base editor systems. In some embodiments, the base editor system comprises one or more base editor systems in conjunction with a single guide polynucleotide or a plurality of guide polynucleotides. In some embodiments, the editing is in conjunction with one or more guide polynucleotide with a single base editor system. In some embodiments, the editing is in conjunction with at least one guide polynucleotide that does not require a PAM sequence to target binding to a target polynucleotide sequence or with at least one guide polynucleotide that requires a PAM sequence to target binding to a target polynucleotide sequence, or with a mix of at least one guide polynucleotide that does not require a PAM sequence to target binding to a target polynucleotide sequence and at least one guide polynucleotide that does require a PAM sequence to target binding to a target polynucleotide sequence. It should be appreciated that the characteristics of the multiplex editing using any of the base editors as described herein can be applied to any of combination of the methods of using any of the base editors provided herein. It should also be appreciated that the editing can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors. In some embodiments, the base editor system capable of multiplex editing comprising one of the ABE8 base editor variants described herein has higher multiplex editing efficiency compared to the base editor system capable of multiplex editing comprising one of ABE7 base editors. In some embodiments, the base editor system capable of multiplex editing comprising one of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% higher multiplex editing efficiency compared the base editor system capable of multiplex editing comprising one of ABE7 base editors. In some embodiments, the base editor system capable of multiplex editing comprising one of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 4.0 fold, at least 4.5 fold, at least 5.0 fold, at least 5.5 fold, or at least 6.0 fold higher multiplex editing efficiency compared the base editor system capable of multiplex editing comprising one of ABE7 base editors. In some embodiments, use of a base editor system capable of multiplex editing of a plurality of nucleobase pairs in one or more genes described herein does not comprise a risk or occurrence of chromosomal translocations.

### Expression of Fusion Proteins or Complexes in a Host Cell

Polypeptides of the present disclosure may be expressed in virtually any host cell of interest, including mammalian cells (e.g., human cells). In some embodiments, the host cell is an immune cell (e.g., T- or NK-cell). In some embodiments, the host cell is an allogeneic immune cell (e.g., T- or NK-cell). In some embodiments, the host cell is a CAR-T cell.

For example, a DNA encoding a polypeptide of the present disclosure can be cloned by designing suitable primers for the upstream and downstream of CDS based on the cDNA sequence The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A DNA encoding a protein domain described herein can be obtained by chemically synthesizing the DNA, or by connecting synthesized partly overlapping oligoDNA short chains by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding a nucleic acid sequence-recognizing module and/or a nucleic acid base converting enzyme can be produced, for example, by linking the DNA to the downstream of a promoter in a suitable expression vector. As the expression vector, animal cell expression plasmids (e.g., pA1-11, pXT1, pRc/CMV, pRc/RSV, pcDNAI/Neo); and animal virus vectors such as retrovirus, vaccinia virus, lentivirus, adenovirus and the like are used.

In some embodiments, the disclosure provides methods for introducing a heterologous polynucleotide into a cell of interest. In embodiments, the methods do not involve the use of a viral particle and/or viral vector. For example, the disclosure provides a method of inserting a heterologous polynucleotide into the genome of a cell using a nuclease (e.g., Cas9 or Cas12 (e.g., Cas12b)) ribonucleoprotein complex (RNP)-DNA template complex where an RNP including a nuclease domain (e.g., Cas9 or Cas12) and a guide RNA, where the guide RNA specifically hybridizes to a target region of the genome of the cell, and where the nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In embodiments, the DNA template is a single-stranded circular DNA template. In embodiments, the molar ratio of RNP to DNA template in the complex is from about 3:1 to about 100:1.

In some embodiments, the DNA template is a linear DNA template. In some embodiments, the DNA template is a circular DNA template (e.g., a single-stranded circular DNA template). In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In some embodiments, the nucleic acid sequence is inserted into the genome of the cell (e.g., T cell) by introducing a vector, for example, a viral or non-viral vector, comprising the nucleic acid. Examples of viral vectors include, but are not limited to, adeno-associated viral (AAV) vectors, retroviral vectors or lentiviral vectors. In some embodiments, the lentiviral vector is an integrase-deficient lentiviral vector.

In some embodiments, the nucleic acid sequence is inserted into the genome of the cell (e.g., T cell) via non-viral delivery. In non-viral delivery methods, the nucleic acid can be naked DNA, or in a non-viral plasmid or vector.

In some embodiments, the nucleic acid is inserted into the cell by introducing into the cell, (a) a targeted nuclease (e.g., a Cas9 or Cas12) that cleaves a target region to create an insertion site in the genome of the T cell; and (b) the nucleic acid sequence, wherein the nucleic acid sequence is incorporated into the insertion site by HDR.

In some cases, the nucleic acid sequence is introduced into the cell as a linear DNA template. In some cases, the nucleic acid sequence is introduced into the cell as a double-stranded DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA. In some cases, the DNA template is a double-stranded or single-stranded plasmid or mini-circle. Methods for integrating such templates are known in the art and described, for example, in US Patent Publication Nos. 20190388469, 20210388362, 20210207174, 20210353678, 20200362355, and 20210228631, which are incorporated herein by reference. See also, Roth, T. L et al., Reprogramming human T cell function and specificity with non-viral genome targeting. Nat. Lett. 559, 405-409 (2018); Ferenczi et al., Nat Commun 12, 6751 (2021). https://doi.org/10.1038/s41467-021-27004-1; Zhang et al., Homology-based repair induced by CRISPR-Cas nucleases in mammalian embryo genome editing. Protein Cell (2021). https://doi.org/10.1007/s13238-021-00838-7 Regarding the promoter to be used, any promoter appropriate for a host to be used for gene expression can be used. In a conventional method using double-stranded breaks, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SRu promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like are preferable.

Expression vectors for use in the present invention, besides those mentioned above, can comprise an enhancer, a splicing signal, a terminator, a polyA addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the polypeptides disclosed herein. A polypeptide of the present disclosure can be intracellularly expressed by introducing into the cell an expression vector comprising a nucleic acid sequence encoding the polypeptide.

Animal cells contemplated in the present disclosure include, but are not limited to, cell lines such as monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used. All the above-mentioned host cells may be haploid (monoploid), or polyploid (e.g., diploid, triploid, tetraploid, etc.). Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present disclosure, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium is preferably about 6 to about 8. The culture is performed at generally about 30° C. to about 40° C. Where necessary, aeration and stirring may be performed.

When a higher eukaryotic cell, such as animal cell, is used as a host cell, a DNA encoding a base editing system of the present disclosure is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Alternatively, the above-mentioned inductive promoter can also be utilized as a vector removal mechanism when higher eukaryotic cells, such as animal cells, and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Delivery System

The suitability of nucleobase editors to target one or more nucleotides in a polynucleotide sequence (e.g., a gene) is evaluated as described herein. In one embodiment, a single cell of interest is transfected, transduced, or otherwise modified with a nucleic acid molecule or molecules encoding a base editing system described herein together with a small amount of a vector encoding a reporter (e.g., GFP). These cells can be any cell line known in the art, including immune cells (e.g., T- or NK-cells), or immortalized human cell lines, such as 293T, K562 or U20S. Alternatively, primary cells (e.g., human immune cells) may be used. Cells may also be obtained from a subject or individual, such as from tissue biopsy, surgery, blood, plasma, serum, or other biological fluid. Such cells may be relevant to the eventual cell target.

Delivery may be performed using a viral vector. In one embodiment, transfection may be performed using lipid transfection (such as Lipofectamine or Fugene) or by electroporation. Following transfection, expression of a reporter (e.g., GFP) can be determined either by fluorescence microscopy or by flow cytometry to confirm consistent and high levels of transfection. These preliminary transfections can comprise different nucleobase editors to determine which combinations of editors give the greatest activity. The system can comprise one or more different vectors. In one embodiment, the base editor is codon optimized for expression of the desired cell type, preferentially a eukaryotic cell, preferably a mammalian cell or a human cell.

The activity of the nucleobase editor is assessed as described herein, i.e., by sequencing the genome of the cells to detect alterations in a target sequence. For Sanger sequencing, purified PCR amplicons are cloned into a plasmid backbone, transformed, miniprepped and sequenced with a single primer. Sequencing may also be performed using next generation sequencing (NGS) techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). The fusion proteins or complexes that induce the greatest levels of target specific alterations in initial tests can be selected for further evaluation.

In particular embodiments, the nucleobase editors are used to target polynucleotides of interest. In one embodiment, a nucleobase editor of the present disclosure is delivered to cells (e.g., immune cells (e.g., T- or NK-cells)) in conjunction with one or more guide RNAs that are used to target one or more nucleic acid sequences of interest within the genome of a cell, thereby altering the target gene(s) (e.g., β2M, TAP1, TAP2, Tapasin, and/or CD58). In some embodiments, a base editor is targeted by one or more guide RNAs to introduce one or more edits to the sequence of one or more genes of interest (e.g., immune cells (e.g., T- or NK-cells)). In some embodiments, the one or more edits to the sequence of one or more genes of interest decrease or eliminate expression of the protein encoded by the gene in the host cell (e.g., immune cells (e.g., T- or NK-cells)). In some embodiments, expression of one or more proteins encoded by one or more genes of interest (e.g., β2M, TAP1, TAP2, Tapasin, and/or CD58) is completely knocked out or eliminated in the host cell (e.g., immune cells (e.g., T- or NK-cells)).

In some embodiments, the host cell is selected from a human cell, or mammalian cell in vitro or in vivo. In some embodiments, the host cell is an immune cell. In some embodiments, the immune cell is a T cell. In some embodiments, the immune cell is a NK cell. In some embodiments, one or more edits are introduced into one or more genes of the immune cell selected from β2M, TAP1, TAP2, Tapasin, and CD58.

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions.

Nucleic acids encoding cytidine or adenosine base editors can be delivered directly to cells (e.g., immune cells) as naked DNA or RNA, for instance by means of transfection or electroporation, or can be conjugated to molecules (e.g., N-acetylgalactosamine) promoting uptake by the target cells. Nucleic acid vectors, such as the vectors described herein can also be used. In particular embodiments, a polynucleotide, e.g. a mRNA encoding a base editor or a functional component thereof may be co-electroporated with a combination of multiple guide RNAs as described herein.

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g. lipid and/or polymer) nanoparticles are suitable for use as delivery vehicles in certain embodiments of this disclosure. Exemplary lipids for use in nanoparticle formulations, and/or gene transfer are shown in Table 16 (below).

TABLE 16
Lipids used for gene transfer.

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 17 lists exemplary polymers for use in gene transfer and/or nanoparticle formulations.

TABLE 17
Polymers used for gene transfer.

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly (propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D, L-lactic-co-glycolic acid) | PLGA |
| Poly(N-ethyl-4-vinylpyridinium bromide) | |
| Poly (phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE−EA |
| Chitosan | |
| Galactosylated chitosan | |
| N-Dodacylated chitosan | |
| Histone | |
| Collagen | |
| Dextran-spermine | D-SPM |

Table 18 summarizes delivery methods for a polynucleotide encoding a fusion protein or complex described herein.

TABLE 18
Delivery methods.

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of base editor system components or nucleic acids encoding such components, for example, a polynucleotide programmable nucleotide binding domain (e.g., Cas9) such as, for example, Cas9 or variants thereof, and a gRNA targeting a nucleic acid sequence of interest, may be accomplished by delivering the ribonucleoprotein (RNP) to cells. The RNP comprises a polynucleotide programmable nucleotide binding domain (e.g., Cas9), in complex with the targeting gRNA. RNPs or polynucleotides described herein may be delivered to cells using known methods, such as electroporation, nucleofection, or cationic lipid-mediated methods, for example, as reported by Zuris, J. A. et al., 2015, Nat. Biotechnology, 33(1):73-80, which is incorporated by reference in its entirety. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

Nucleic acid molecules encoding a base editor system can be delivered directly to cells (e.g., T- or NK-cells)) as naked DNA or RNA by means of transfection or electroporation, for example, or can be conjugated to molecules (e.g., N-acetylgalactosamine) promoting uptake by the target cells. Vectors encoding base editor systems and/or their components can also be used. In particular embodiments, a polynucleotide, e.g. a mRNA encoding a base editor system or a functional component thereof, may be co-electroporated with one or more guide RNAs as described herein.

Nucleic acid vectors can comprise one or more sequences encoding a domain of a fusion protein or complex described herein. A vector can also encode a protein component of a base editor system operably linked to a nuclear localization signal, nucleolar localization signal, or mitochondrial localization signal. As one example, a vector can include a Cas9 coding sequence that includes one or more nuclear localization sequences (e.g., a nuclear localization sequence from SV40), and one or more deaminases.

The vector can also include any suitable number of regulatory/control elements, e.g., promoters, enhancers, introns, polyadenylation signals, Kozak consensus sequences, or internal ribosome entry sites (IRES). These elements are well known in the art.

Vectors according to this disclosure include recombinant viral vectors. Exemplary viral vectors are set forth herein above. Other viral vectors known in the art can also be used. In addition, viral particles can be used to deliver base editor system components in nucleic acid and/or protein form. For example, “empty” viral particles can be assembled to contain a base editor system or component as cargo. Viral vectors and viral particles can also be engineered to incorporate targeting ligands to alter target tissue specificity.

Vectors described herein may comprise regulatory elements to drive expression of a base editor system or component thereof. Such vectors include adeno-associated viruses with inverted long terminal repeats (AAV ITR). The use of AAV-ITR can be advantageous for eliminating the need for an additional promoter element, which can take up space in the vector. The additional space freed up can be used to drive the expression of additional elements, such as a guide nucleic acid or a selectable marker. ITR activity can be used to reduce potential toxicity due to over expression.

Any suitable promoter can be used to drive expression of a base editor system or component thereof and, where appropriate, the guide nucleic acid. For ubiquitous expression, promoters include CMV, CBA, CBH, CAG, CBh, PGK, SV40, Ferritin heavy or light chains. For brain or other CNS cell expression, suitable promoters include: SynapsinI for all neurons, CaMKIIalpha for excitatory neurons, GAD67 or GAD65 or VGAT for GABAergic neurons. For liver cell expression, suitable promoters include the Albumin promoter. For lung cell expression, suitable promoters include SP-B. For endothelial cells, suitable promoters include ICAM. For hematopoietic cell expression suitable promoters include IFNbeta or CD45. For osteoblast expression suitable promoters can include OG-2.

In some embodiments, a base editor system of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters, such as U6 or H1 Use of Pol II promoter and intronic cassettes to express gRNA Adeno Associated Virus (AAV).

In particular embodiments, a fusion protein or complex of the present disclosure is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, and variants thereof), or a suitable capsid protein of any viral vector. Thus, in some aspects, the disclosure relates to the viral delivery of a fusion protein or complex. Examples of viral vectors include retroviral vectors (e.g. Maloney murine leukemia virus, MML-V), adenoviral vectors (e.g. AD100), lentiviral vectors (HIV and FIV-based vectors), herpesvirus vectors (e.g. HSV-2).

In some aspects, the methods described herein for editing specific genes in a cell can be used to genetically modify an immune cell (e.g., T- or NK-cell). In some aspects, the methods described herein for editing specific genes in an immune cell can be used to genetically modify a CAR-T cell. Such CAR-T cells, and methods to produce such CAR-T cells are described in International Application Nos. PCT/US2016/060736, PCT/US2016/060734, PCT/US2016/034873, PCT/US2015/040660, PCT/EP2016/055332, PCT/IB2015/058650, PCT/EP2015/067441, PCT/EP2014/078876, PCT/EP2014/059662, PCT/IB2014/061409, PCT/US2016/019192, PCT/US2015/059106, PCT/US2016/052260, PCT/US2015/020606, PCT/US2015/055764, PCT/CN2014/094393, PCT/US2017/059989, PCT/US2017/027606, and PCT/US2015/064269, the contents of each is hereby incorporated in its entirety.

### Viral Vectors

Base editors and constructs (e.g., soluble or membrane-bound HLA-G and/or HLA-E trimers and/or dimers) described herein can be delivered with a viral vector. In some embodiments, a base editor, and/or construct disclosed herein can be encoded on a nucleic acid that is contained in a viral vector. In some embodiments, one or more components of the base editor system and/or construct can be encoded on one or more viral vectors. For example, a base editor and guide nucleic acid, and/or construct can be encoded on a single viral vector. In other embodiments, the base editor and guide nucleic acid, and/or construct are encoded on different viral vectors. In either case, the base editor and guide nucleic acid, and/or construct can each be operably linked to a promoter and terminator. The combination of components encoded on a viral vector can be determined by the cargo size constraints of the chosen viral vector.

The use of RNA or DNA viral based systems for the delivery of a base editor or construct takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing or expression of a construct, the expression of the base editor and optional guide nucleic acid, and/or construct can be driven by a cell-type specific promoter. Non-limiting examples of suitable promoters include promoter elongation factor-1 alpha.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a base editor or construct of the present disclosure is of sufficient size so as to enable efficient packaging and delivery into a target cell via a retroviral vector. In some embodiments, a base editor or construct is of a size so as to allow efficient packing and delivery even when expressed together with a guide nucleic acid and/or other components of a targetable nuclease system.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, Adeno-associated virus (“AAV”) vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. AAV vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

In some embodiments, AAV vectors are used to transduce a cell of interest with a polynucleotide encoding a base editor or base editor system as provided herein. AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein or complex of the present disclosure and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor or construct which is shorter in length than conventional base editors. In some examples, the base editors or constructs are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, lentiviral vectors are used to transduce an immune cell (e.g., T- or NK-cell) with a polynucleotide encoding a base editor or base editor system, and/or construct as provided herein. In some embodiments, lentiviral vectors are used to transduce an modified immune cell (e.g., T- or NK-cell) with a chimeric antigen receptor (CAR). Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 μl Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the base editor-coding sequence and/or the guide nucleic acid can be modified to include one or more modified nucleoside e.g. using pseudo-U or 5-Methyl-C.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

A fragment (e.g., a functional fragment) of a fusion protein or complex of the present disclosure can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas9 nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In some embodiments, the nucleic acid sequence is inserted into the genome of the cell (e.g., T cell) via non-viral delivery. In non-viral delivery methods, the nucleic acid can be naked DNA, or in a non-viral plasmid or vector.

In some embodiments, the nucleic acid is inserted into the cell by introducing into the cell, (a) a targeted nuclease that cleaves a target region to create an insertion site in the genome of the T cell; and (b) the nucleic acid sequence, wherein the nucleic acid sequence is incorporated into the insertion site by HDR.

In some cases, the nucleic acid sequence is introduced into the cell as a linear DNA template. In some cases, the nucleic acid sequence is introduced into the cell as a double-stranded DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA. In some cases, the DNA template is a double-stranded or single-stranded plasmid or mini-circle.

In other embodiments, a single-stranded DNA (ssDNA) can produce efficient HDR with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (QssDNA) donors.

Methods for integrating such templates are known in the art and described, for example, in US Patent Publications No. 20190388469, 20210388362, 20210207174, 20210353678, 20200362355, and 20210228631, which are incorporated herein by reference. See also, Roth, T. L et al., Reprogramming human T cell function and specificity with non-viral genome targeting. Nat. Lett. 559, 405-409 (2018); Ferenczi et al., Nat Commun 12, 6751 (2021). https://doi.org/10.1038/s41467-021-27004-1; Zhang et al., Homology-based repair induced by CRISPR-Cas nucleases in mammalian embryo genome editing. Protein Cell (2021). https://doi.org/10.1007/s13238-021-00838-7.

### Inteins

Inteins (intervening protein) are auto-processing domains found in a variety of diverse organisms, which carry out a process known as protein splicing. Protein splicing is a multi-step biochemical reaction comprised of both the cleavage and formation of peptide bonds. While the endogenous substrates of protein splicing are proteins found in intein-containing organisms, inteins can also be used to chemically manipulate virtually any polypeptide backbone.

In protein splicing, the intein excises itself out of a precursor polypeptide by cleaving two peptide bonds, thereby ligating the flanking extein (external protein) sequences via the formation of a new peptide bond. This rearrangement occurs post-translationally (or possibly co-translationally). Intein-mediated protein splicing occurs spontaneously, requiring only the folding of the intein domain.

About 5% of inteins are split inteins, which are transcribed and translated as two separate polypeptides, the N-intein and C-intein, each fused to one extein. Upon translation, the intein fragments spontaneously and non-covalently assemble into the canonical intein structure to carry out protein splicing in trans. The mechanism of protein splicing entails a series of acyl-transfer reactions that result in the cleavage of two peptide bonds at the intein-extein junctions and the formation of anew peptide bond between the N- and C-exteins. This process is initiated by activation of the peptide bond joining the N-extein and the N-terminus of the intein. Virtually all inteins have a cysteine or serine at their N-terminus that attacks the carbonyl carbon of the C-terminal N-extein residue. This N to O/S acyl-shift is facilitated by a conserved threonine and histidine (referred to as the TXXH motif), along with a commonly found aspartate, which results in the formation of a linear (thio)ester intermediate. Next, this intermediate is subject to trans-(thio)esterification by nucleophilic attack of the first C-extein residue (+1), which is a cysteine, serine, or threonine. The resulting branched (thio)ester intermediate is resolved through a unique transformation: cyclization of the highly conserved C-terminal asparagine of the intein. This process is facilitated by the histidine (found in a highly conserved HNF motif) and the penultimate histidine and may also involve the aspartate. This succinimide formation reaction excises the intein from the reactive complex and leaves behind the exteins attached through a non-peptidic linkage. This structure rapidly rearranges into a stable peptide bond in an intein-independent fashion. In some embodiments, the split intein is selected from Gp41.1, IMPDH.1, NrdJ.1 and Gp41.8 (Carvajal-Vallejos, Patricia et al. “Unprecedented rates and efficiencies revealed for new natural split inteins from metagenomic sources.” J. Biol. Chem., vol. 287,34 (2012)).

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limitine examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377. Inteins suitable for use in embodiments of the present disclosure and methods for use thereof are described in U.S. Pat. No. 10,526,401, International Patent Application Publication No. WO 2013/045632, and in U.S. Patent Application Publication No. US 2020/0055900, the full disclosures of which are incorporated herein by reference in their entireties by reference for all purposes.

Further non-limiting examples of amino acid and nucleotide sequences for N-inteins and C-inteins suitable for use as intein pairs include those with at least 85% sequence identity to an amino acid or nucleotide sequence listed in the following Tables 20A-20C, or a fragments thereof that function as part of a split intein pair.

TABLE 20A
Exemplary amino acid and nucleotide sequences
for N-Inteins.

| | | SEQ |
|---|---|---|
| N- | | ID |
| Intein | Amino Acid or Nucleotide Sequence | NO |
| Cfa | TGCCTGAGCTACGATACCGAGATCCTGACCGTG | 389 |
| (GEP) | GAATACGGCTTCCTGCCTATCGGCAAGATCGTC | |
| (nucle- | GAGGAACGGATCGAGTGCACAGTGTACACCGTG | |
| otide | GATAAGAATGGCTTCGTGTACACCCAGCCTATC | |
| se- | GCTCAGTGGCACAACAGAGGCGAGCAAGAGGTG | |
| quence) | TTCGAGTACTGCCTGGAAGATGGCAGCATCATC | |
| | CGGGCCACCAAGGACCACAAGTTTATGACCACC | |
| | GACGGCCAGATGCTGCCCATCGACGAGATCTTT | |
| | GAGAGAGGCCTGGACCTGAAACAGGTGGACGGA | |
| | CTGCCT | |
| Cfa | CLSYDTEILTVEYGFLPIGKIVEERIECTVYTV | 390 |
| (GEP) | DKNGFVYTQPIAQWHNRGEQEVFEYCLEDGSII | |
| (amino | RATKDHKFMTTDGQMLPIDEIFERGLDLKQVDG | |
| acid | LP | |
| se- | | |
| quence) | | |
| Gp41.1 | TGTCTGGACCTCAAGACCCAAGTGCAGACACCT | 391 |
| (nucle- | CAGGGCATGAAAGAGATTAGCAATATCCAGGTG | |
| otide | GGCGACCTGGTCCTGAGCAACACCGGCTACAAC | |
| se- | GAGGTGCTGAACGTGTTCCCTAAGTCCAAGAAG | |
| quence) | AAATCTTATAAGATCACCCTGGAAGATGGCAAG | |
| | GAAATCATCTGCAGCGAGGAACACCTGTTCCCC | |
| | ACCCAGACCGGCGAGATGAACATCAGCGGCGGA | |
| | CTGAAGGAGGGCATGTGCCTGTACGTGAAGGAG | |
| Gp41.1 | CLDLKTQVQTPQGMKEISNIQVGDLVLSNTGYN | 392 |
| (amino | EVLNVFPKSKKKSYKITLEDGKEIICSEEHLFP | |
| acid | TQTGEMNISGGLKEGMCLYVKE | |
| se- | | |
| quence) | | |
| Gp41.8 | TGCCTGAGCCTGGACACCATGGTGGTGACAAAC | 393 |
| (nucle- | GGCAAGGCCATCGAGATCAGAGATGTGAAGGTG | |
| otide | GGAGATTGGCTGGAAAGCGAATGTGGCCCAGTG | |
| se- | CAGGTTACAGAGGTGCTGCCTATCATCAAGCAG | |
| quence) | CCTGTCTTTGAGATTGTGCTGAAAAGCGGAAAA | |
| | AAGATCCGGGTGTCCGCTAATCACAAGTTCCCC | |
| | ACCAAGGACGGCCTCAAGACCATCAACAGCGGC | |
| | CTGAAGGTGGGCGACTTCCTGAGAAGCAGAGCC | |
| | AAG | |
| Gp41.8 | CLSLDTMVVTNGKAIEIRDVKVGDWLESECGPV | 394 |
| (amino | QVTEVLPIIKQPVFEIVLKSGKKIRVSANHKFP | |
| acid | TKDGLKTINSGLKVGDFLRSRAK | |
| se- | | |
| quence) | | |
| IMPDH.1 | TGTTTTGTGCCTGGCACCCTGGTGAACACAGAG | 395 |
| (nucle- | AATGGCCTGAAGAAAATCGAGGAAATCAAGGTG | |
| otide | GGCGACAAGGTGTTCAGCCATACAGGCAAGCTG | |
| se- | CAGGAGGTGGTGGACACCCTGATCTTCGACCGG | |
| quence) | GACGAGGAAATCATCTCTATCAACGGCATTGAT | |
| | TGCACCAAGAACCACGAGTTCTACGTGATCGAT | |
| | AAGGAAAACGCTAATAGAGTGAACGAGGACAAC | |
| | ATCCACCTCTTCGCCAGATGGGTCCACGCCGAG | |
| | GAACTGGATATGAAAAAGCACCTGCTGATCGAG | |
| | CTGGAA | |
| IMPDH.1 | CFVPGTLVNTENGLKKIEEIKVGDKVFSHTGKL | 396 |
| (amino | QEVVDTLIFDRDEEIISINGIDCTKNHEFYVID | |
| acid | KENANRVNEDNIHLFARWVHAEELDMKKHLLIE | |
| se- | LE | |
| quence) | | |
| NrdJ.1 | TGCCTGGTGGGCTCTAGCGAGATTATCACAAGA | 397 |
| (nucle- | AACTACGGCAAGACCACCATCAAGGAAGTGGTC | |
| otide | GAGATCTTCGACAACGACAAGAATATCCAGGTG | |
| se- | CTGGCCTTCAACACCCACACCGATAATATCGAG | |
| quence) | TGGGCCCCTATCAAGGCCGCTCAGCTGACCAGA | |
| | CCTAACGCCGAGCTGGTTGAACTGGAAATCGAC | |
| | ACCCTGCACGGCGTGAAAACAATCCGGTGCACC | |
| | CCTGACCACCCCGTGTACACCAAGAACAGAGGC | |
| | TACGTGCGGGCCGACGAGCTGACAGATGATGAC | |
| | GAGCTCGTGGTGGCTATC | |
| NrdJ.1 | CLVGSSEIITRNYGKTTIKEVVEIFDNDKNIQV | 398 |
| (amino | LAFNTHTDNIEWAPIKAAQLTRPNAELVELEID | |
| acid | TLHGVKTIRCTPDHPVYTKNRGYVRADELTDDD | |
| se- | ELVVAI | |
| quence) | | |
| Npu | TGCCTGAGCTACGAGACAGAGATCCTGACCGTG | 399 |
| (nucle- | GAATATGGCCTGCTGCCAATCGGAAAGATCGTG | |
| otide | GAAAAGCGGATCGAGTGCACCGTCTACAGCGTG | |
| se- | GACAACAACGGAAATATCTATACACAGCCTGTG | |
| quence) | GCCCAATGGCACGACCGGGGCGAACAGGAGGTG | |
| | TTTGAGTACTGCCTGGAAGATGGTTCTCTGATT | |
| | AGAGCCACCAAGGACCACAAGTTCATGACCGTC | |
| | GACGGCCAGATGCTGCCCATCGACGAAATCTTC | |
| | GAGCGGGAACTCGACCTGATGAGAGTGGATAAC | |
| | CTGCCCAAT | |
| Npu | CLSYETEILTVEYGLLPIGKIVEKRIECTVYSV | 400 |
| (amino | DNNGNIYTQPVAQWHDRGEQEVFEYCLEDGSLI | |
| acid | RATKDHKFMTVDGQMLPIDEIFERELDLMRVDN | |
| se- | LPN | |
| quence) | | |
| Cfa N- | CLSYDTEILTVEYGFLPIGKIVEERIECTVYTV | 401 |
| intein | DKNGFVYTQPIAQWHNRGEQEVFEYCLEDGSII | |
| | RATKDHKFMTTDGQMLPIDEIFERGLDLKQVDG | |
| | LP | |
| Npu N- | CLSYETEILTVEYGLLPIGKIVEKRIECTVYSV | 402 |
| intein | DNNGNIYTQPVAQWHDRGEQEVFEYCLEDGSLI | |
| | RATKDHKFMTVDGQMLPIDEIFERELDLMRVDN | |
| | LPN | |

TABLE 20B
Further exemplary amino acid and nucleotide
sequences for N-Inteins.

| N- | | SEQ |
|---|---|---|
| Intein- | | ID |
| SC | Amino Acid or Nucleotide Sequence | NO |
| Gp41.1 | ACAAGAAGCGGATACTGTCTGGACCTCAAGACC | 413 |
| (nucle- | CAAGTGCAGACACCTCAGGGCATGAAAGAGATT | |
| otide | AGCAATATCCAGGTGGGCGACCTGGTCCTGAGC | |
| se- | AACACCGGCTACAACGAGGTGCTGAACGTGTTC | |
| quence) | CCTAAGTCCAAGAAGAAATCTTATAAGATCACC | |
| | CTGGAAGATGGCAAGGAAATCATCTGCAGCGAG | |
| | GAACACCTGTTCCCCACCCAGACCGGCGAGATG | |
| | AACATCAGCGGCGGACTGAAGGAGGGCATGTGC | |
| | CTGTACGTGAAGGAG | |
| Gp41.1 | TRSGYCLDLKTQVQTPQGMKEISNIQVGDLVLS | 414 |
| (amino | NTGYNEVLNVFPKSKKKSYKITLEDGKEIICSE | |
| acid | EHLFPTQTGEMNISGGLKEGMCLYVKE | |
| se- | | |
| quence) | | |
| Gp41.8 | TCTCAGCTGAACCGGTGCCTGAGCCTGGACACC | 415 |
| (nucle- | ATGGTGGTGACAAACGGCAAGGCCATCGAGATC | |
| otide | AGAGATGTGAAGGTGGGAGATTGGCTGGAAAGC | |
| se- | GAATGTGGCCCAGTGCAGGTTACAGAGGTGCTG | |
| quence) | CCTATCATCAAGCAGCCTGTCTTTGAGATTGTG | |
| | CTGAAAAGCGGAAAAAAGATCCGGGTGTCCGCT | |
| | AATCACAAGTTCCCCACCAAGGACGGCCTCAAG | |
| | ACCATCAACAGCGGCCTGAAGGTGGGCGACTTC | |
| | CTGAGAAGCAGAGCCAAG | |
| Gp41.8 | SQLNRCLSLDTMVVTNGKAIEIRDVKVGDWLES | 416 |
| (amino | ECGPVQVTEVLPIIKQPVFEIVLKSGKKIRVSA | |
| acid | NHKFPTKDGLKTINSGLKVGDFLRSRAK | |
| se- | | |
| quence) | | |
| IMPDH.1 | GGCATCGGCGGAGGATGTTTTGTGCCTGGCACC | 417 |
| (nucle- | CTGGTGAACACAGAGAATGGCCTGAAGAAAATC | |
| otide | GAGGAAATCAAGGTGGGCGACAAGGTGTTCAGC | |
| se- | CATACAGGCAAGCTGCAGGAGGTGGTGGACACC | |
| quence) | CTGATCTTCGACCGGGACGAGGAAATCATCTCT | |
| | ATCAACGGCATTGATTGCACCAAGAACCACGAG | |
| | TTCTACGTGATCGATAAGGAAAACGCTAATAGA | |
| | GTGAACGAGGACAACATCCACCTCTTCGCCAGA | |
| | TGGGTCCACGCCGAGGAACTGGATATGAAAAAG | |
| | CACCTGCTGATCGAGCTGGAA | |
| IMPDH.1 | GIGGGCFVPGTLVNTENGLKKIEEIKVGDKVFS | 418 |
| (amino | HTGKLQEVVDTLIFDRDEEIISINGIDCTKNHE | |
| acid | FYVIDKENANRVNEDNIHLFARWVHAEELDMKK | |
| se- | HLLIELE | |
| quence) | | |
| NrdJ.1 | GGAACAAACCCATGTTGCCTGGTGGGCTCTAGC | 419 |
| (nucle- | GAGATTATCACAAGAAACTACGGCAAGACCACC | |
| otide | ATCAAGGAAGTGGTCGAGATCTTCGACAACGAC | |
| se- | AAGAATATCCAGGTGCTGGCCTTCAACACCCAC | |
| quence) | ACCGATAATATCGAGTGGGCCCCTATCAAGGCC | |
| | GCTCAGCTGACCAGACCTAACGCCGAGCTGGTT | |
| | GAACTGGAAATCGACACCCTGCACGGCGTGAAA | |
| | ACAATCCGGTGCACCCCTGACCACCCCGTGTAC | |
| | ACCAAGAACAGAGGCTACGTGCGGGCCGACGAG | |
| | CTGACAGATGATGACGAGCTCGTGGTGGCTATC | |
| NrdJ.1 | GTNPCCLVGSSEIITRNYGKTTIKEVVEIFDND | 420 |
| (amino | KNIQVLAFNTHTDNIEWAPIKAAQLTRPNAELV | |
| acid | ELEIDTLHGVKTIRCTPDHPVYTKNRGYVRADE | |
| se- | LTDDDELVVAI | |
| quence) | | |

TABLE 20C
Exemplary amino acid and nucleotide
sequences for C-Inteins.

| | | SEQ |
|---|---|---|
| C- | Amino Acid or Nucleotide | ID |
| Intein | Sequence | NO |
| Cfa(GEP) | GTCAAGATCATCAGCAGAAAGAGCCTGGGC | 421 |
| (nucle- | ACCCAGAACGTGTACGATATCGGAGTGGGC | |
| otide | GAGCCCCACAACTTTCTGCTCAAGAATGGC | |
| se- | CTGGTGGCCAGCAAC | |
| quence) | | |
| Cfa(GEP) | VKIISRKSLGTQNVYDIGVGEPHNFLLKNG | 422 |
| (amino | LVASN | |
| acid | | |
| se- | | |
| quence) | | |
| Gp41.1 | ATGATGCTGAAAAAGATCCTGAAGATCGAG | 423 |
| (nucle- | GAACTGGATGAGAGAGAGCTGATCGACATC | |
| otide | GAAGTGTCTGGCAATCACCTGTTCTACGCC | |
| se- | AACGACATCCTGACCCACAACAGC | |
| quence) | | |
| Gp41.1 | MMLKKILKIEELDERELIDIEVSGNHLFYA | 424 |
| (amino | NDILTHNS | |
| acid | | |
| se- | | |
| quence) | | |
| Gp41.8 | ATGTGCGAAATCTTCGAGAACGAGATTGAT | 425 |
| (nucle- | TGGGACGAAATCGCCTCTATCGAGTACGTG | |
| otide | GGCGTGGAAGAGACAATCGACATCAACGTG | |
| se- | ACCAACGACAGACTGTTTTTCGCCAATGGC | |
| quence) | ATCCTGACCCACAACAGC | |
| Gp41.8 | MCEIFENEIDWDEIASIEYVGVEETIDINV | 426 |
| (amino | TNDRLFFANGILTHNS | |
| acid | | |
| se- | | |
| quence) | | |
| IMPDH.1 | ATGAAATTCAAGCTGAAGGAAATCACCAGC | 427 |
| (nucle- | ATCGAGACAAAGCACTACAAGGGCAAGGTG | |
| otide | CACGATCTGACCGTGAACCAGGACCACAGC | |
| se- | TACAACGTCAGAGGCACCGTGGTGCATAAT | |
| quence) | TCT | |
| IMPDH.1 | MKFKLKEITSIETKHYKGKVHDLTVNQDHS | 428 |
| (amino | YNVRGTVVHNS | |
| acid | | |
| se- | | |
| quence) | | |
| NrdJ.1 | ATGGAAGCCAAGACCTACATCGGCAAGCTG | 429 |
| (nucle- | AAATCTAGAAAGATCGTGTCCAACGAGGAT | |
| otide | ACATACGACATCCAGACCAGCACCCACAAT | |
| se- | TTCTTCGCCAACGACATCCTGGTGCACAAC | |
| quence) | AGC | |
| NrdJ.1 | MEAKTYIGKLKSRKIVSNEDTYDIQTSTHN | 430 |
| (amino | FFANDILVHNS | |
| acid | | |
| se- | | |
| quence) | | |
| Npu | ATGATCAAGATCGCCACAAGAAAGTACCTG | 431 |
| (nucle- | GGCAAGCAGAACGTGTACGACATCGGCGTG | |
| otide | GAGAGAGACCACAACTTCGCCCTGAAGAAC | |
| se- | GGCTTTATCGCCTCTAAT | |
| quence) | | |
| Npu | MIKIATRKYLGKQNVYDIGVERDHNFALKN | 432 |
| (amino | GFIASN | |
| acid | | |
| se- | | |
| quence) | | |
| Cfa C- | MVKIISRKSLGTQNVYDIGVGEPHNFLLKN | 433 |
| intein | GLVASN | |
| (amino | | |
| acid | | |
| se- | | |
| quence) | | |
| Npu C- | IKIATRKYLGKQNVYDIGVERDHNFALKNG | 434 |
| intein | FIASN | |
| (amino | | |
| acid | | |
| se- | | |
| quence) | | |

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. The mechanism of intein-mediated protein splicing for joining the proteins the inteins are fused to (e.g., split Cas9) is known in the art, e.g., as described in Shah et al., Chem Sci. 2014; 5(1):446-461, incorporated herein by reference. Methods for designing and using inteins are known in the art and described, for example by WO2014004336, WO2017132580, WO2013045632A1, US20150344549, and US20180127780, each of which is incorporated herein by reference in their entirety.

In some embodiments, a portion or fragment of a nuclease (e.g., Cas9) is fused to an intein. The nuclease can be fused to the N-terminus or the C-terminus of the intein. In some embodiments, a portion or fragment of a fusion protein or complex is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, an N-terminal fragment of a base editor (e.g., ABE, CBE) is fused to a split intein-N and a C-terminal fragment is fused to a split intein-C. In some embodiments, an N-terminal fragment of a base editor (e.g., ABE, CBE) is fused to a split intein-N and a C-terminal fragment is fused to a split intein-C. In some embodiments, an N-terminal fragment of a nucleic acid programmable DNA binding protein (napDNAbp) domain (e.g., Cas9) is fused to a split intein-N and a C-terminal fragment is fused to a split intein-C. In some embodiments, an N-terminal fragment of a deaminase domain (e.g. adenosine or cytidine deaminase) fused to a split intein-N and a C-terminal fragment is fused to a split intein-C.

These fragments are then packaged into two or more AAV vectors. In some embodiments, the N-terminus of an intein is fused to the C-terminus of a fusion protein and the C-terminus of the intein is fused to the N-terminus of an AAV capsid protein.

In one embodiment, inteins are utilized to join fragments or portions of a cytidine or adenosine base editor protein that is grafted onto an AAV capsid protein. The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, which are indicated in capital letters in the sequence below (called the “Cas9 reference sequence”).

| (SEQ ID NO: 197) | |
|---|---|
| 1 | mdkkysigld igtnsvgwav itdeykvpsk kfkvlgntdr hsikknliga llfdsgetae |
| 61 | atrlkrtarr rytrrknric ylqeifsnem akvddsffhr leesflveed kkherhpifg |
| 121 | nivdevayhe kyptiyhlrk klvdstdkad lrliylalah mikfrghfli egdlnpdnsd |
| 181 | vdklfiqlvq tynqlfeenp inasgvdaka ilsarlsksr rlenliaqlp gekknglfgn |
| 241 | lialslgltp nfksnfdlae daklqlskdt ydddldnlla qigdqyadlf laaknlsdai |
| 301 | llSdilrvnT eiTkaplsas mikrydehhq dltllkalvr qqlpekykei ffdqSkngya |
| 361 | gyidggasqe efykfikpil ekmdgteell vklnredllr kqrtfdngsi phqihlgelh |
| 421 | ailrrqedfy pflkdnreki ekiltfripy yvgplArgnS rfAwmTrkSe eTiTpwnfee |
| 481 | vvdkgasaqs fiermtnfdk nlpnekvlpk hsllyeyftv yneltkvkyv tegmrkpafl |
| 541 | sgeqkkaivd llfktnrkvt vkqlkedyfk kieCfdSvei sgvedrfnAS lgtyhdllki |
| 601 | ikdkdfldne enedilediv ltltlfedre mieerlktya hlfddkvmkq lkrrrytgwg |
| 661 | rlsrklingi rdkqsgktil dflksdgfan rnfmqlihdd sltfkediqk aqvsgqgdsl |
| 721 | hehianlags paikkgilqt vkvvdelvkv mgrhkpeniv iemarenqtt qkgqknsrer |
| 781 | mkrieegike lgsqilkehp ventqlqnek lylyylqngr dmyvdqeldi nrlsdydvdh |
| 841 | ivpqsflkdd sidnkvltrs dknrgksdnv pseevvkkmk nywrqllnak litqrkfdnl |
| 901 | tkaergglse ldkagfikrq lvetrqitkh vaqildsrmn tkydendkli revkvitlks |
| 961 | klvsdfrkdf qfykvreinn yhhahdayln avvgtalikk ypklesefvy gdykvydvrk |
| 1021 | miakseqeig katakyffys nimnffktei tlangeirkr plietngetg eivwdkgrdf |
| 1081 | atvrkvlsmp qvnivkktev qtggfskesi lpkrnsdkli arkkdwdpkk yggfdsptva |
| 1141 | ysvlvvakve kgkskklksv kellgitime rssfeknpid fleakgykev kkdliiklpk |
| 1201 | yslfelengr krmlasagel qkgnelalps kyvnflylas hyeklkgspe dneqkqlfve |
| 1261 | qhkhyldeii eqisefskrv iladanldkv lsaynkhrdk pireqaenii hlftltnlga |
| 1321 | paafkyfdtt idrkrytstk evldatlihq sitglyetri dlsqlggd. |

### Pharmaceutical Compositions

In some aspects, the present disclosure provides a pharmaceutical composition comprising any of the genetically modified immune cells, polynucleotides, vectors, base editors, base editor systems, guide polynucleotides, fusion proteins, complexes, or the fusion protein-guide polynucleotide complexes described herein. More specifically, provided herein are pharmaceutical compositions comprising a genetically modified allogeneic immune cell (e.g., T- or NK-cell), or a population of such immune cells, wherein said modified immune cell has at least one edited gene to provide increased persistence, resistance to immune rejection, or decreased risk of eliciting a host-versus-graft reaction, or a combination thereof. In some embodiments, the at least one edited gene is beta-2 microglobulin (β2M), Transporter Associated with Antigen Processing I (TAP1), Transporter Associated with Antigen Processing II (TAP2), and/or Tapasin. In some embodiments, TAP1 and/or TAP2 is edited. In some embodiments, beta-2 microglobulin (β2M), Transporter Associated with Antigen Processing I (TAP1), Transporter Associated with Antigen Processing II (TAP2), and/or Tapasin are edited in combination with one or more genes selected from TCRα Chain (TRAC), Cluster of Differentiation 58 (CD58), and/or Class II, Major Histocompatibility Complex Transactivator (CIITA). In some embodiments, one or more inhibitory receptors selected from Human Leukocyte Antigen-E (HLA-E), Human Leukocyte Antigen-G (HLA-G), Programmed Death Ligand 1 (PD-L1), and/or Cluster of Differentiation 47 (CD47) are overexpressed in the genetically modified allogeneic immune cell (e.g., T- or NK-cell).

In some cases, a composition of the disclosure contains a delivery vehicle (e.g., a vector, such as a lipid nanoparticle or a viral vector).

In embodiments, a pharmaceutical composition of the disclosure comprises a modified immune cell that has a reduced level of, lacks, or has virtually undetectable levels of one or more of the polypeptides listed herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In addition to a modified immune cell, or population thereof, and a carrier, the pharmaceutical compositions of the present disclosure can include at least one additional therapeutic agent useful in the treatment of disease. For example, some embodiments of the pharmaceutical composition described herein further comprises a chemotherapeutic agent. In some embodiments, the pharmaceutical composition further comprises a cytokine peptide or a nucleic acid sequence encoding a cytokine peptide. In some embodiments, the pharmaceutical compositions comprising the modified immune cell or population thereof can be administered separately from an additional therapeutic agent.

One consideration concerning the therapeutic use of genetically modified cells of the present disclosure is the quantity of cells necessary to achieve an optimal or satisfactory effect. The quantity of cells to be administered may vary for the subject being treated. In one embodiment, between 10⁴ to 10¹⁰, between 10⁵ to 10⁹, or between 10⁶ and 10⁸ genetically modified cells of the present disclosure are administered to a human subject. In some embodiments, at least about 1×10⁸, 2×10⁸, 3×10⁸, 4×10⁸, and 5×10⁸ genetically modified immune cells of the present disclosure are administered to a human subject. Determining the precise effective dose may be based on factors for each individual subject, including their size, age, sex, weight, and condition. Dosages can be readily ascertained by those skilled in the art from this disclosure and the knowledge in the art.

The skilled artisan can readily determine the number of cells and amount of optional additives, vehicles, and/or carriers in compositions and to be administered in methods of the present disclosure. Typically, additives (in addition to the cell(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (see, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et al., 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethylenegly col (PEG) coating (Zhang Y. P. et al., Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the present disclosure in lyophilized form and (b) a second container containing a pharmaceutically acceptable diluent (e.g., sterile used for reconstitution or dilution of the lyophilized compound of the present disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the present disclosure. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts, for example, for veterinary use.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. See also PCT application PCT/US2010/055131 (Publication number WO2011/053982 A8, filed Nov. 2, 2010), incorporated in its entirety herein by reference, for additional suitable methods, reagents, excipients and solvents for producing pharmaceutical compositions comprising a nuclease.

Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions. In some embodiments, the pharmaceutical compositions of the present disclosure can be used to treat any disease or condition that is responsive to autologous or allogeneic immune cell immunotherapy. In some embodiments, the modified immune cell is administered to an allogenic host, wherein the modified immune cell has no rejection by the host. In some embodiments, the allogenic modified immune cell induces negligible or minimum rejection by the host.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the present disclosure comprise expressing or introducing into a cell a base editor polypeptide and one or more guide RNAs capable of targeting a nucleic acid molecule encoding at least one polypeptide. In some embodiments, the methods of treatment comprise administering to a subject in need thereof a pharmaceutical composition comprising a population of modified immune cells having at least one edited gene (e.g., beta-2 microglobulin (β2M), Transporter Associated with Antigen Processing I (TAP1), Transporter Associated with Antigen Processing II (TAP2), Tapasin, and/or CD58), wherein the at least one edited gene reduces or inactivates surface HLA class-I expression and provides increased persistence, resistance to immune rejection, or decreased risk of eliciting a host-versus-graft reaction, or a combination thereof. In some embodiments, the modified immune cell is an allogeneic immune cell.

In some embodiments, the methods of treatment comprise administering to a subject an effective amount of a modified immune effector cell (e.g., allogeneic modified immune cell) or a population thereof that has at least one single target nucleobase modification in one or more of a beta-2 microglobulin (β2M), Transporter Associated with Antigen Processing I (TAP1), Transporter Associated with Antigen Processing II (TAP2), Tapasin, and/or CD58 gene sequence and, in some embodiments, further has reduced or inactivated surface HLA class-I expression. In some embodiments, the modified immune effector cell further overexpresses one or more inhibitory receptors selected from Human Leukocyte Antigen-E (HLA-E), Human Leukocyte Antigen-G (HLA-G), Programmed Death Ligand 1 (PD-L1), and/or Cluster of Differentiation 47 (CD47). In some embodiments, the modified immune cell further overexpresses inhibitory receptors Human Leukocyte Antigen-E (HLA-E), Human Leukocyte Antigen-G (HLA-G), Programmed Death Ligand 1 (PD-L1), and Cluster of Differentiation 47 (CD47). In some embodiments, the modified immune effector cell (e.g., allogeneic modified immune cell) further has at least one single target nucleobase modification in a nucleic acid molecule encoding at least one additional gene sequence or regulatory element thereof. In some embodiments, the at least one additional gene sequence or regulatory element is TCRα Chain (TRAC), Cluster of Differentiation 58 (CD58), and/or Class II, Major Histocompatibility Complex Transactivator (CIITA).

In one embodiment, a subject is administered at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells. In particular embodiments, about 1×10⁷ cells to about 1×10⁹ cells, about 2×10⁷ cells to about 0.9×10⁹ cells, about 3×10⁷ cells to about 0.8×10⁹ cells, about 4×10⁷ cells to about 0.7×10⁹ cells, about 5×10⁷ cells to about 0.6×10⁹ cells, or about 5×10⁷ cells to about 0.5×10⁹ cells are administered to the subject.

In one embodiment, a subject is administered at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight. In particular embodiments, about 1×10⁶ cells/kg of bodyweight to about 1×10⁸ cells/kg of bodyweight, about 2×10⁶ cells/kg of bodyweight to about 0.9×10⁸ cells/kg of bodyweight, about 3×10⁶ cells/kg of bodyweight to about 0.8×10⁸ cells/kg of bodyweight, about 4×10⁶ cells/kg of bodyweight to about 0.7×10⁸ cells/kg of bodyweight, about 5×10⁶ cells/kg of bodyweight to about 0.6×10⁸ cells/kg of bodyweight, or about 5×10⁶ cells/kg of bodyweight to about 0.5×10⁸ cells/kg of bodyweight are administered to the subject.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more. In any of such methods, the methods may comprise administering to the subject an effective amount of a modified immune cell or a base editor system or polynucleotide encoding such system. In any of such methods, the methods may comprise administering one or more doses of an effective amount of the modified immune cells per day. In any of such methods, the methods may comprise administering two or more doses of an effective amount of the modified immune cells per day. In any of such methods, the methods may comprise administering three or more doses of an effective amount of the modified immune cells per day. In any of such methods, the methods may comprise administering one or more doses of an effective amount of the modified immune cells per week. In any of such methods, the methods may comprise administering two or more doses of an effective amount of the modified immune cells per week. In any of such methods, the methods may comprise administering three or more doses of an effective amount of the modified immune cells per week. In any of such methods, the methods may comprise administering one or more doses of an effective amount of the modified immune cells per month. In any of such methods, the methods may comprise administering two or more doses of an effective amount of the modified immune cells per month. In any of such methods, the methods may comprise administering three or more doses of an effective amount of the modified immune cells per month.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

In some embodiments, a composition described herein (e.g., edited cell, base editor system) is administered in a dosage that is about 0.5-30 mg per kilogram body weight of the human subject. In another embodiment, the amount of the composition administered is about 0.5-20 mg per kilogram body weight of the human subject. In another embodiment, the amount of the composition administered is about 0.5-10 mg per kilogram body weight of the human subject. In another embodiment, the amount of the composition administered is about 0.04 mg, about 0.08 mg, about 0.16 mg, about 0.32 mg, about 0.64 mg, about 1.25 mg, about 1.28 mg, about 1.92 mg, about 2.5 mg, about 3.56 mg, about 3.75 mg, about 5.0 mg, about 7.12 mg, about 7.5 mg, about 10 mg, about 14.24 mg, about 15 mg, about 20 mg, or about 30 mg per kilogram body weight of the human subject. In another embodiment, the amount of the compo composition und administered is about 1.92 mg, about 3.75 mg, about 7.5 mg, about 15.0 mg, or about 30.0 mg per kilogram body weight of the human subject and the composition is administered two times a week. In another embodiment, the amount of the composition administered is about 1.28 mg, about 2.56 mg, about 5.0 mg, about 10 mg, or about 20 mg per kilogram body weight of the human subject and the composition is administered two times a week. In another embodiment, the amount of the composition administered is about 1.92 mg, about 3.75 mg, about 7.5 mg, about 15.0 mg, or about 30.0 mg per kilogram body weight of the human subject and the composition is administered once a week. In another embodiment, the amount of the composition administered is about 1.28 mg, about 2.56 mg, about 5.0 mg, about 10 mg, or about 20 mg per kilogram body weight of the human subject and the composition is administered once a week. In another embodiment, the amount of the composition administered is about 1.92 mg, about 3.75 mg, about 7.5 mg, about 15.0 mg, or about 30.0 mg per kilogram body weight of the human subject and the composition is administered once a day three, five or seven times in a seven day period. In another embodiment, the composition is administered intravenously once a day, seven times in a seven day period. In another embodiment, the amount of the composition administered is about 1.28 mg, about 2.56 mg, about 5.0 mg, about 10 mg, or about 20 mg per kilogram body weight of the human subject and the composition is administered once a day three, five or seven times in a seven day period. In another embodiment, the composition is administered intravenously once a day, seven times in a seven day period.

In some embodiments, the composition is administered over a period of 0.25 h, 0.5 h, 1 h, 2 h, 3 h, 4 h, 5 h, 6 h, 7 h, 8 h, 9 h, 10 h, 11 h, or 12 h. In another embodiment, the composition is administered over a period of 0.25-2 h. In another embodiment, the composition is gradually administered over a period of 1 h. In another embodiment, the composition is gradually administered over a period of 2 h.

### Kits

The present disclosure provides kits featuring an allogeneic modified immune cell (e.g., T- or NK-cell) as provided herein. In some embodiments, the kit includes an allogeneic modified CAR-T cell as provided herein. In some embodiments, the kit further includes a base editor, base editor system or a polynucleotide encoding a base editor or base editor system as provided herein. In some embodiments, the kit further includes one or more guide polynucleotides (e.g., a guide polynucleotide targeting a genomic sequence) as provided herein. In some embodiments, the base editor system comprises a nucleic acid programmable DNA binding protein (napDNAbp), a deaminase (e.g., cytidine deaminase or adenosine deaminase), and a guide RNA for modifying an allogeneic CAR-T cell. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the base editor is an ABE, CBE or CABE. In some embodiments, the guide RNA is selected from Table TA or Table 1B. In various instances, the kit comprises an edited cell and instructions regarding the use of such cell.

The kits may further comprise written instructions for using the base editor, base editor system and/or allogeneic modified immune cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the present disclosure, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the present disclosure, and are not intended to limit the scope of what the inventors regard as their invention.

### EXAMPLES

### Example 1: Engineered Persistent Allogeneic T Cells

Pre-clinical and clinical data suggest that adoptively transferred allogeneic CAR-T cells exhibit poor persistence in vivo due to recipient/host immune-mediated rejection mechanisms. Host-derived alloreactive T cells recognize allogeneic CAR-T cells as “non-self” by binding to peptide complexed with foreign Human Leukocyte Antigen (HLA) on the surface of CAR-T cells. Approaches to render allogeneic CAR-T cells invisible to host-derived alloreactive T cells include genetic ablation of beta-2-Microglobulin (β2M). β2M forms a heterotrimer with HLA class-I heavy chain and class-I specific peptide to enable antigen presentation to surveilling T cells. However, the presence of surface peptide/HLA class-I negatively regulates Natural Killer (NK) cells by engaging inhibitory Killer Ig-Like Receptors (KIRs). As such, the absence of surface HLA on cells leaves them susceptible to NK cell-mediated lysis.

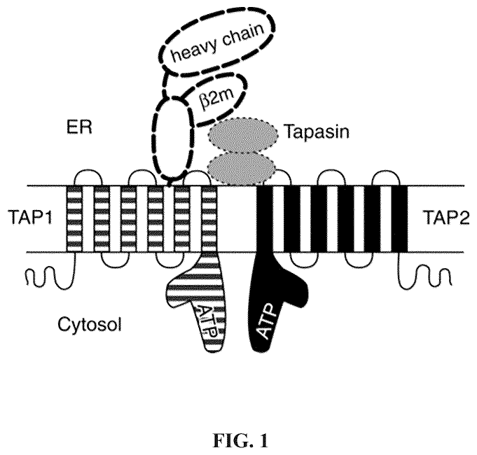

Generating HLA-deficient allogeneic CAR-T cells can overcome host-derived alloreactive T cell-mediated rejection, however, the likelihood exists that the CAR-T cells will be eliminated by other arms of the host immune system, namely NK cells. In order to overcome this obstacle, surface peptide/HLA expression will be attenuated on allogeneic CAR-T cells to a level that is below the threshold to activate host-derived alloreactive T cells and above the threshold to inhibit host-derived NK cells. Cellular targets for genetic disruption include components of the Peptide Loading Complex (PLC), such as TAP1, TAP2, Tapasin, and β2M (FIG. 1). Members of the PLC are responsible for proper assembly of mature peptide/HLA class-I complexes. Naturally occurring mutations have been described within genes encoding for proteins of the PLC that can modulate surface peptide/HLA class-I expression. For instance, individuals with Bare Lymphocyte Syndrome (BLS) Type I exhibit a marked reduction, but not complete ablation, of surface HLA expression.

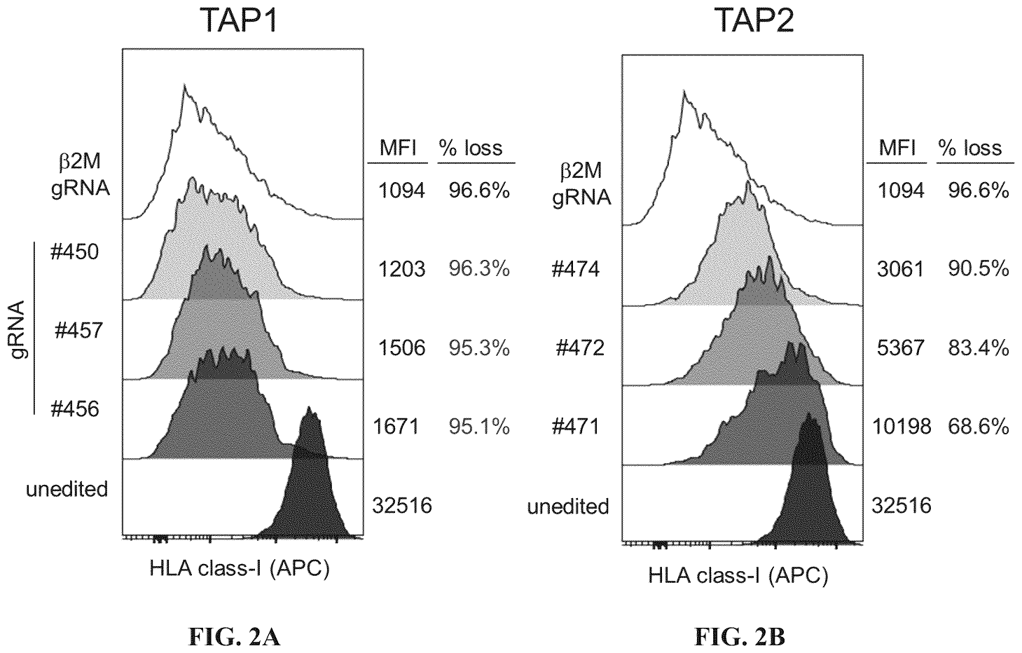

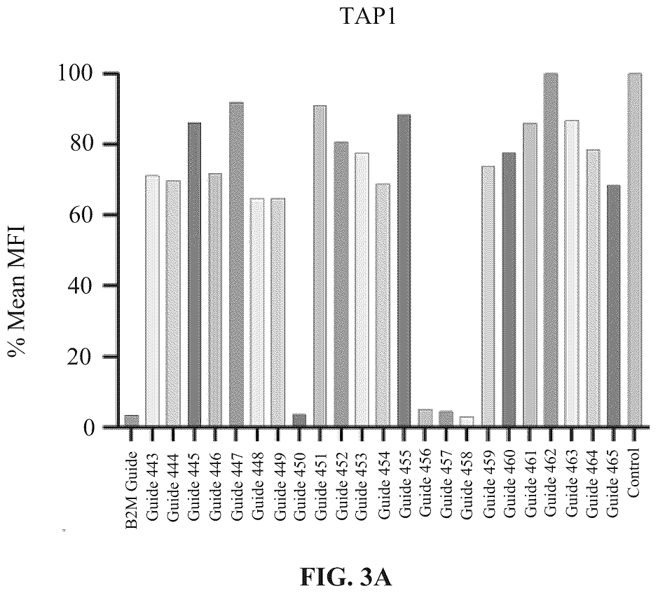

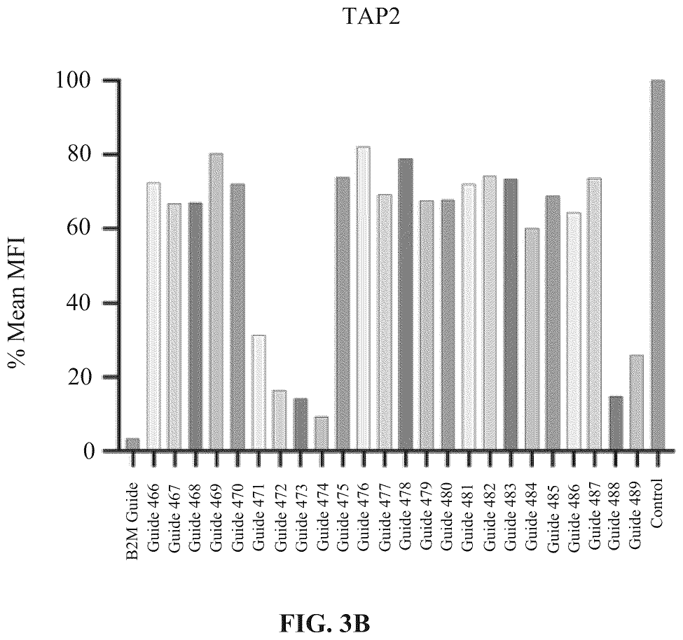

To test whether base editing modulates surface HLA class-I expression, splice sites were targeted for base editing using a rBE4 base editor and guide RNAs 443-465 for TAP1 and guide RNAs 466-489 for TAP2 (Table TA). Base edits in TAP1 resulted in near ablation of surface HLA class-I expression using β2M gRNA as a complete knock-out control, while base edits in TAP2 show attenuation of surface HLA class-I expression (see FIGS. 2A, 2B, 3A, 3B). Overall, these data support that TAP1 and TAP2 base editing modulates surface HLA class-I expression.

### Example 2: Materials & Methods

Modified guides (3X 2′-O-methyl 3′-phosphorothioate 5′ and 3′; commercially available from Agilent) were used for screening purposes. Guides were reconstituted in RNAse free water at 2 μg/μL and frozen prior to screening. mRNA encoding the rBE4 base editor was aliquoted at 2 μg/μL and frozen prior to screening.

Human primary T cells were isolated and activated at 1×10⁶ cells/mL using TransAct (1:100). T cells were activated in serum free media (XF T Cell Expansion Media+10% CTS Immune Cell SR+1% glutamax+1% HEPEs+IL7/IL15). Three (3) days post activation the cells were used for guide screening. A standard Lonza nucleofection protocol was followed (20 μL cassettes): T cells were counted, eluted into P3 buffer with supplement, and screened using 2 μg/μL of CBE mRNA editor and 2 μg/μL of guide per 0.750-1×10⁶ cells/20 μL reaction using standard DH102 protocol. A reliable β2M guide was used as a control for editing efficiency and comparison for the TAP1 and TAP2 guides. After electroporation, cells were plated in 24 well plates in standard serum free media. 72 hours post electroporation, a subset of cells was removed into a 96 well plate, and stained for β2M expression using a flow cytometer, the MQ16 Flow analyzer. Data was analyzed using a commercially available software package for flow cytometry analysis (FlowJo).

### Example 3: Engineered NK Cell Resistant K562 Cells

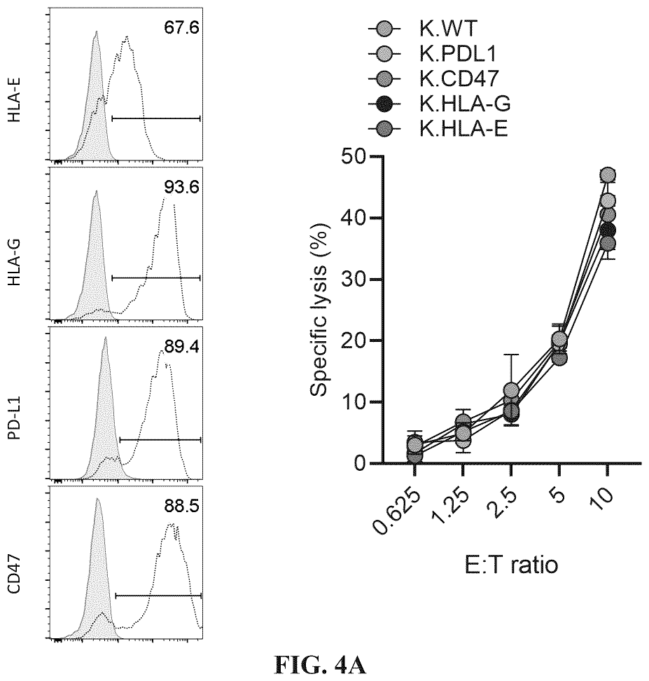

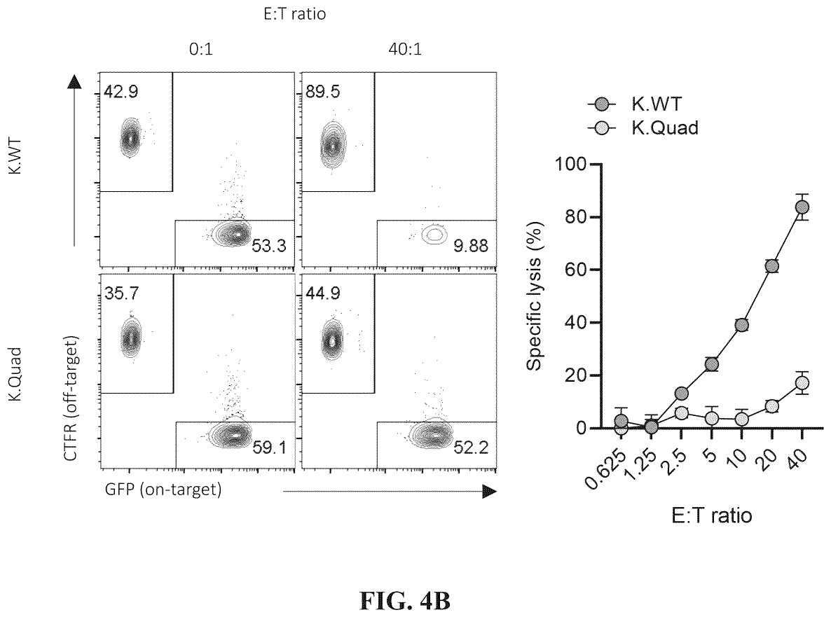

NK cells exhibit a variegated pattern of inhibitory receptors. In order to investigate whether a cell line could be engineered to suppress NK cell lysis in vitro, inhibitory receptors CD47, PD-L1, HLA-G, or HLA-E were overexpressed in K562 cells, which have no surface HLA expression and are susceptible to NK cell lysis (FIG. 4A). Wild-type was used as a control. Expression of each receptor alone did not suppress NK cell lysis as compared to wild-type. To determine if multiple inhibitory receptors would suppress NK cell lysis, all four inhibitory receptors CD47, PD-L1, HLA-G, and HLA-E (K.Quad) were overexpressed in K562 cells (FIG. 4B). NK cell resistance was observed when overexpressing all four receptors in K562 cells.

### Example 4: BLT Humanized Murine Model

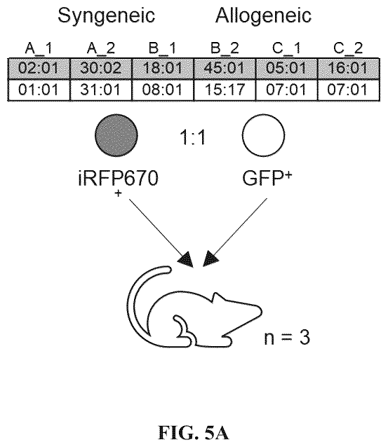

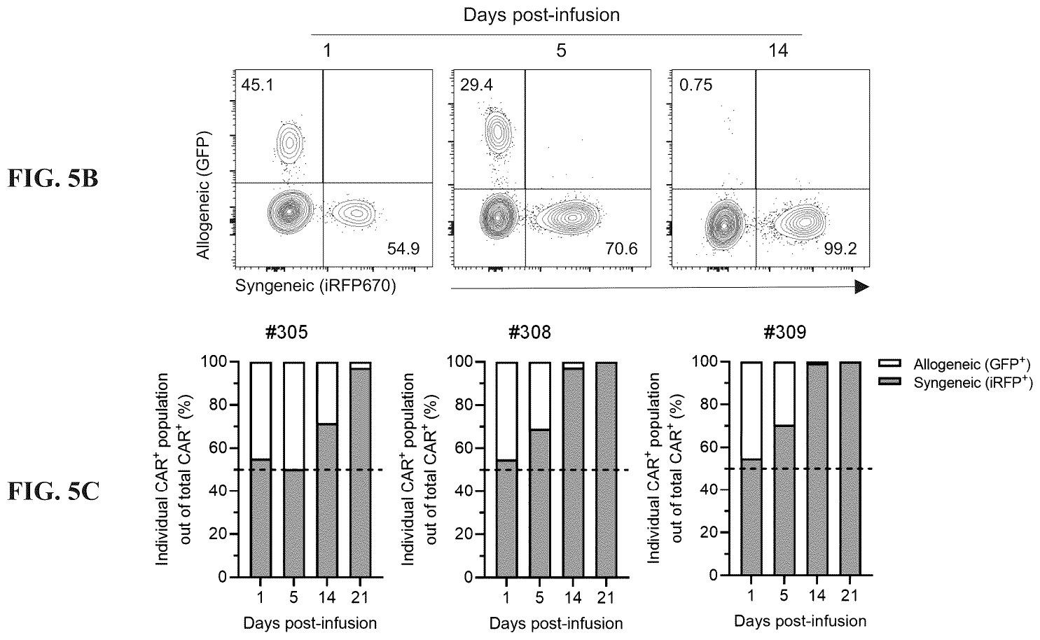

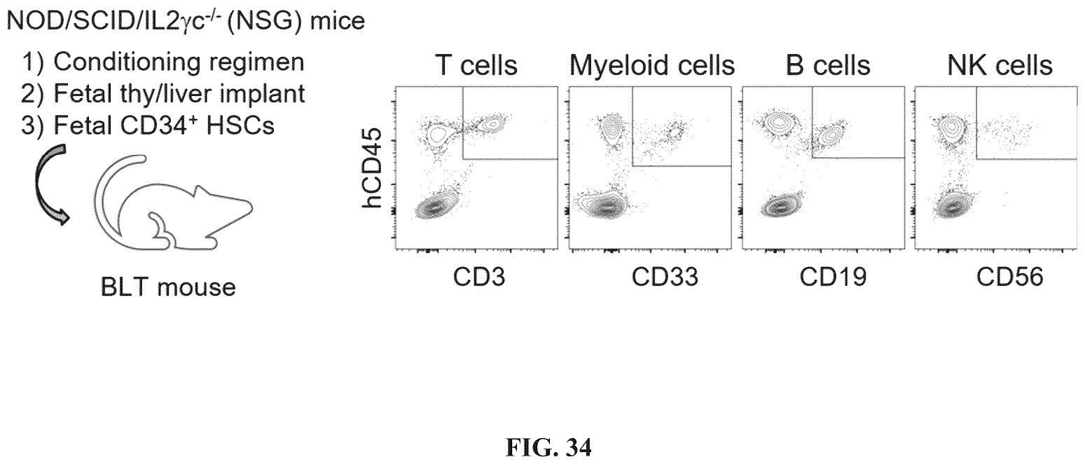

A BLT humanized murine model (NOD/SCID/IL2γc^{-/-} (NSG) mice; Ragon Institute (see FIG. 34) was used to measure the rejection rates of engrafted allogeneic T cells. BLT humanized mice recapitulate both T cell and NK cell mediated allorejection and, therefore, represent a model system for evaluating allogeneic evasion strategies in vivo. To validate the model, T cells were isolated from both syngeneic BLT humanized mice (iRFP670+ cells) and peripheral blood mononuclear cells (PBMCs) of an allogeneic de-identified human donor (GFP+) (FIG. 5A). BLT mice were infused with a mixture (1:1 ratio) of syngeneic BLT-derived T cells and huPBMC-derived T cells.

Persistence of allogeneic cells (GFP) to syngeneic cells (iRFP670) was measured at one (1), five (5), and fourteen (14) days post-infusion (FIGS. 5B and 5C). Individual CAR⁺ populations (#305, #308, or #309) out of the total CAR⁺ (%) of allogeneic cells (GFP) and syngeneic cells (iRFP⁺) was also measured at one (1), five (5), fourteen (14), and twenty-one (21) days post-infusion. By day fourteen (14), allogeneic T cells were cleared, whereas syngeneic T cells persisted indicating that the mode of regulation in the BLT murine model was T-cell mediated.

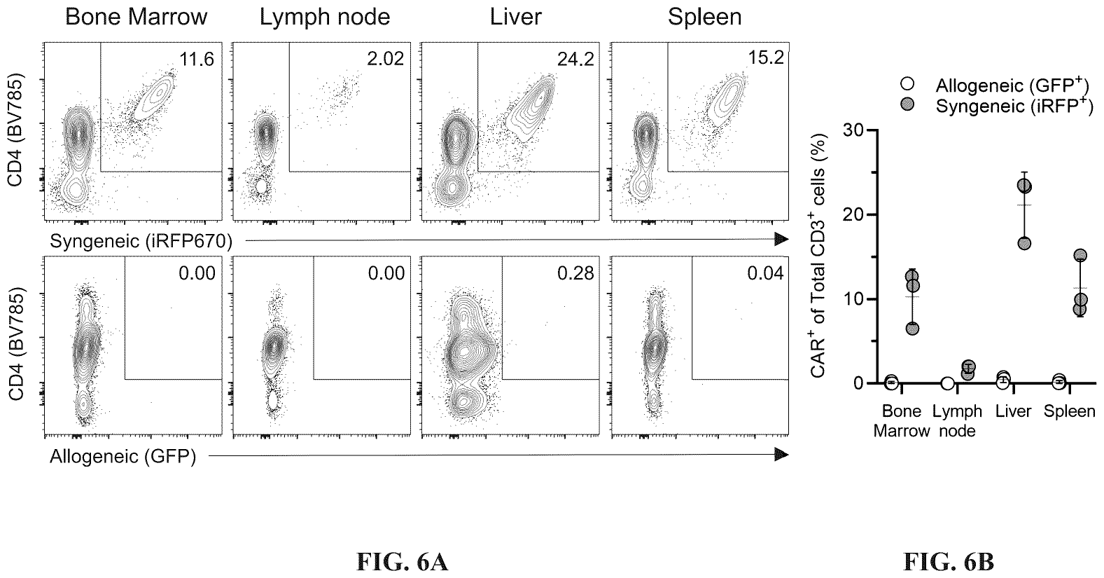

To determine if this result was tissue specific, the presence of allogeneic (GFP) and syngeneic (iRFP670) CAR-T cells was measured in multiple tissue types, including bone marrow, lymph node, liver and spleen in comparison to CD4 (BV785) cells (FIG. 6A). The amount of CAR⁺ syngeneic (iRFP670) cells and allogeneic (GFP) cells out of the total CD3⁺ cells (%) was also measured in bone marrow, lymph node, liver and spleen (FIG. 6B). All tissue types showed a depletion of allogeneic CAR-T cells as compared to syngeneic (iRFP670) CAR-T cells.

### Example 5: Persistent Allogeneic CAR-T Cells in BLT Humanized Mice

To test whether allogeneic CAR-T cells could be engineered to increase persistence in BLT humanized in vivo, T cells were isolated from both syngeneic BLT humanized mice (iRFP670+ cells) and peripheral blood mononuclear cells (PBMCs) of an allogeneic de-identified human donor (GFP+). T cells were pan-stimulated and cultured for 3 days prior to transferring ≤10 million cells per donor source (i.e. BLT- and huPBMC-derived). Activated T cells from each donor source were base-edited using one or more guide RNAs specific to the TCRα chain (TRAC), Beta-2-Microglobulin (β2M), Class II, Major Histocompatibility Complex Transactivator (CIITA), Transporter Associated with Antigen Processing I (TAP1), and Transporter Associated with Antigen Processing II (TAP2) loci. T cells were then recovered in culture for a period of 4-6 hours. Base-edited T cells were cultured for an additional 5-6 days until infusion into recipient BLT mice. Minimum of 2 mice per condition. BLT mice were infused with a mixture (1:1 ratio) of syngeneic BLT-derived T cells and base-edited huPBMC-derived T cells. Mice were bled at day 1 and 7 post-infusion to evaluate ratio of infused cells by flow cytometry. Mice were then bled weekly thereafter until study end-point. Tissue samples were then cryopreserved for downstream analysis.

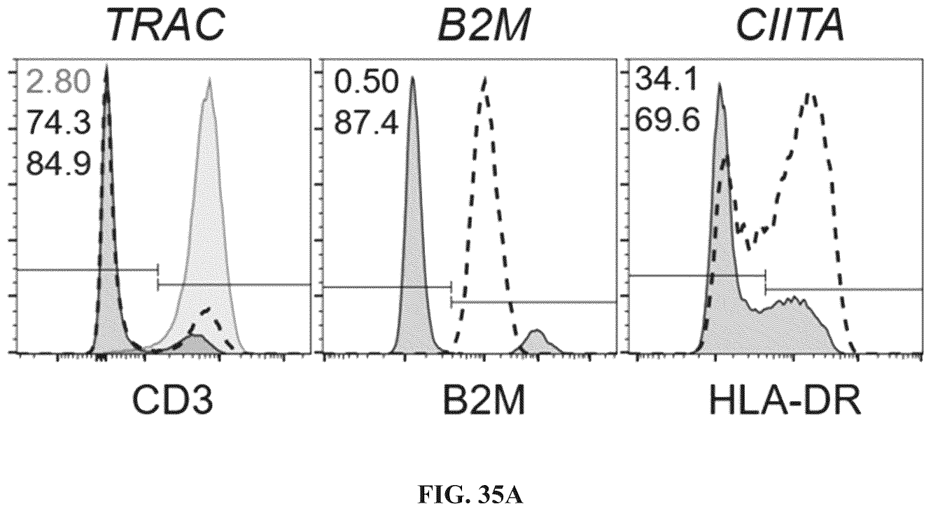

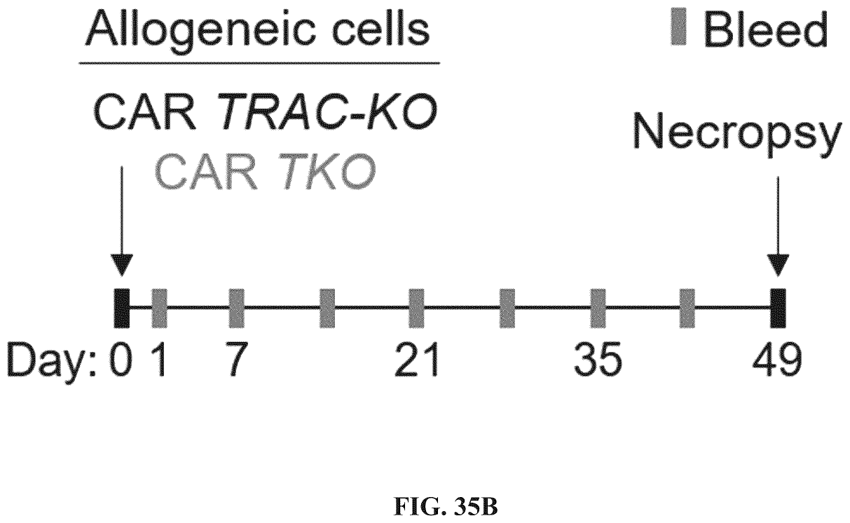

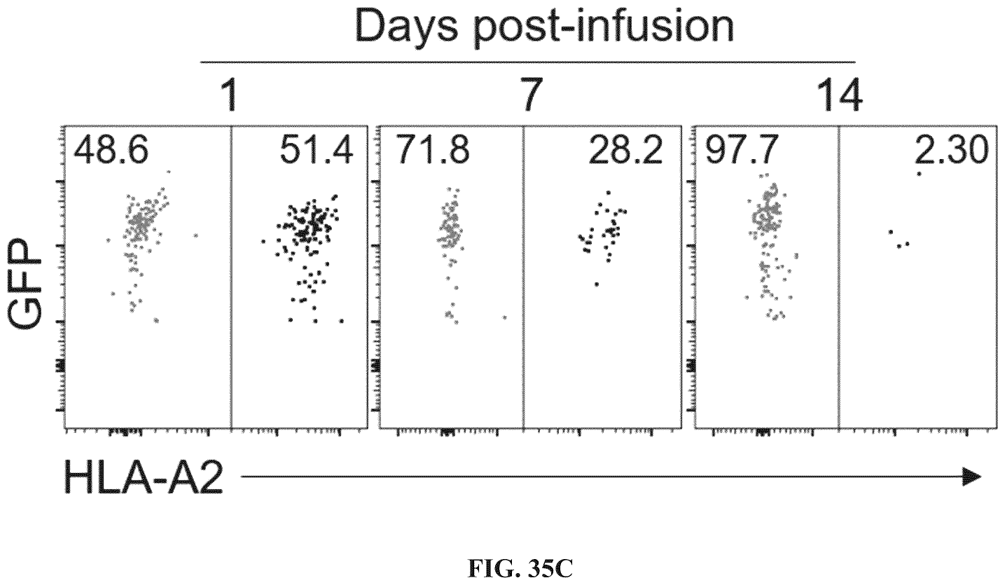

HLA deficient CAR-T cells overcame in vivo T cell-mediated allorejection. Expression of CD3, B2M, and HLA-DR in TRAC knock-out (KO) or TRAC, B2M, and CIITA triple knock-out (TKO) cells was measured using flow cytometry (see FIG. 35A) to confirm that the base editing of the genes was effective in reducing polypeptide expression. To evaluate the impact of the base edits on allorejection, BLT mice were then co-infused with a 1:1 ratio of TRAC-KO and TKO CAR-T cells (see FIG. 35B). Persistence of the TRAC-KO and TKO CAR-T cells post-infusion was measured over time using flow cytometry (FIG. 35C). CAR-T cells having a triple knock-out (TKO) of TRAC, B2M, and CIITA cells persisted for at least 14 days.

### Example 6: HLA Knock-Out CAR-T Cells Overcome Host T Cell-Mediated Allorejection

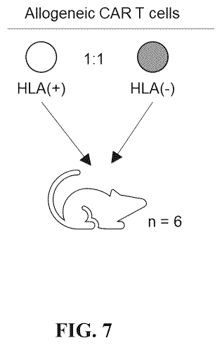

In order to test the mechanism of host T cell-mediated allorejection, HLA+ and HLA-knock-out CAR-T cells were infused in a 1:1 ratio into recipient mice (FIG. 7).

### Example 7: Human IL-15 Infusions Augment NK Cell Reconstitution in BLT Mice

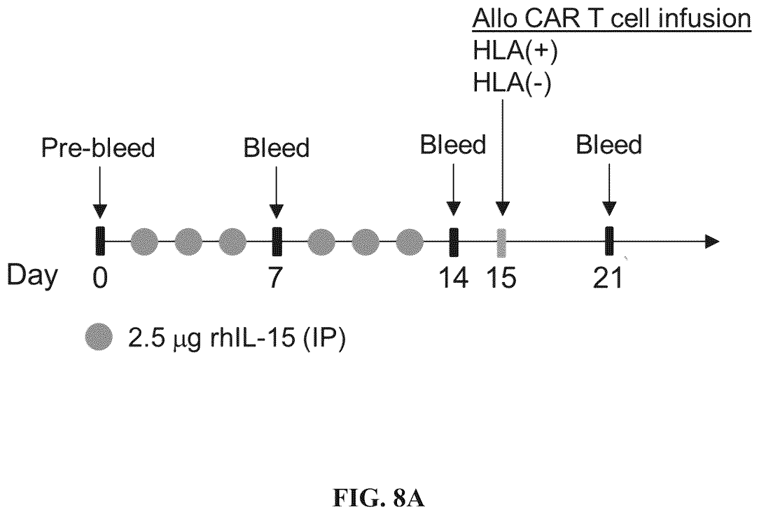

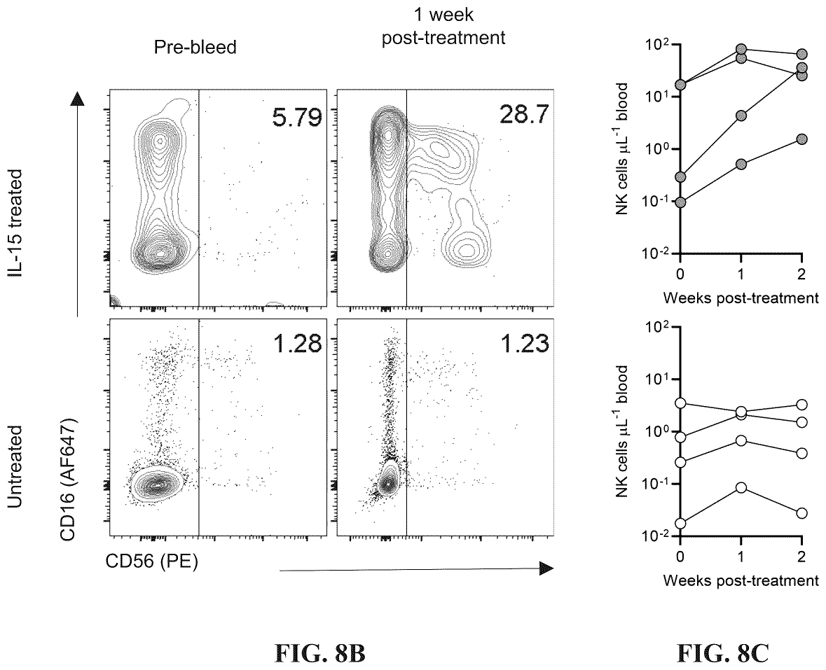

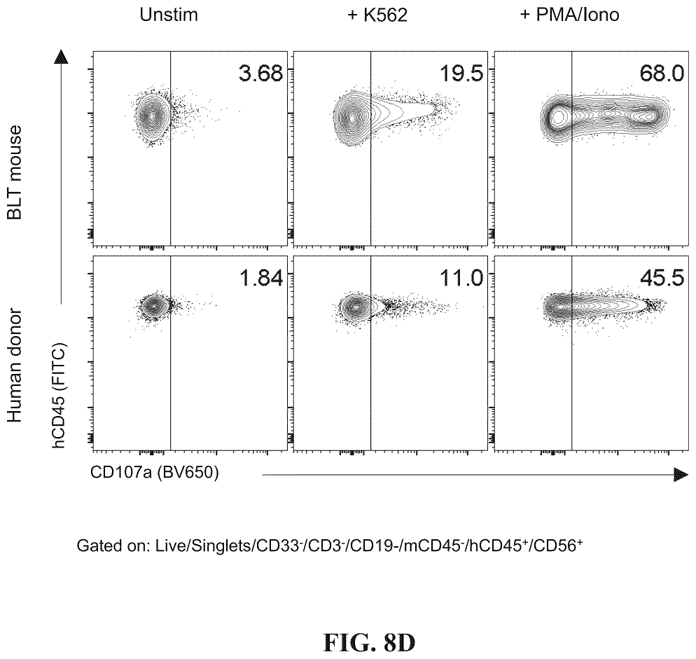

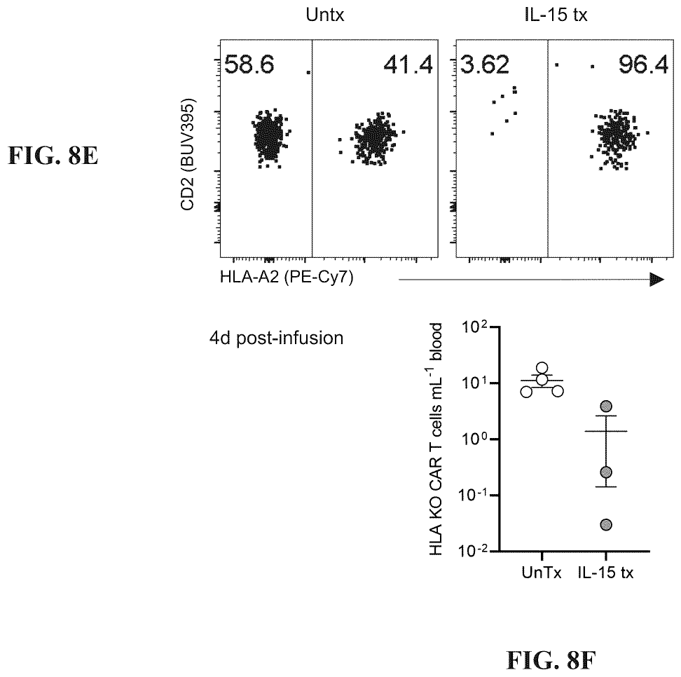

IL-15 is a common γ-chain cytokine that affects T and NK cell homeostasis, proliferation and differentiation. NK cells in BLT mice poorly reconstitute due to lack of sufficient hIL-15 presentation, as well as minimal cross-reactivity with murine IL-15. In order to reconstitute NK cells in BLT mice, BLT mice were infused with 2.5 μg of rhIL-15 via intraperitoneal injection (IP) as depicted in FIG. 8A. NK cells were detected via CD56 expression in BLT mice one week after post-treatment with IL-15 (FIG. 8B) and were able to expand weeks post-treatment (FIG. 8C). Untreated mice were used as a control. These data demonstrate that IL-15 infusions were able to recapitulate NK cells in BLT mice for allorejection studies.

To determine how IL-15 primed NK cells responded to HLA null cells, allogeneic HLA⁺ and HLA⁻ knock-out CAR-T cells were infused in IL-15 infused BLT mice at day 15 as depicted in FIG. 8A. Human donor cells were used for comparison. Unstimulated and K562 cells were used as controls. NK cell functional activity was detected (FIG. 8D) and HLA expression was decreased (FIG. 8E) in BLT mice with IL-15 primed NK cells. The number of HLA⁻ knock-out CAR-T cells was also measured four (4) days post-infusion in BLT mice treated with IL-15 and in untreated mice (FIG. 8F). Overall, these data suggest that IL-15 primed NK cells in BLT mice were able to respond to and clear HLA null cells.

### Example 8: Knock-Out of CD58 Using Cas9, ABE8.20m, or rBE4

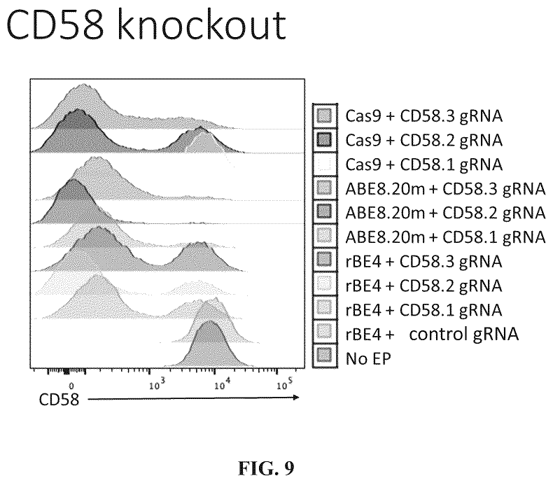

Knocking out CD58 in allogeneic cells is associated with improved immunogenic compatibility of the allogeneic cells. Therefore, gRNAs (CD58.1, CD58.2, and CD58.3 listed in Table 1B) were designed and evaluated for use in knocking out expression of CD58 in human primary T cells. The CD58 gene was edited in the cells using the methods described in Example 2. Successful knock-out of CD58 was detected using flow cytometry (see. FIG. 9). The guide RNAs were effective in combination with each of rBE4, ABE8.20m, and nuclease-active SpCas9 (Cas9) in effecting knock-out or reduced expression of CD58 in the cells.

### Example 9: Gene Modifications to Ablate HLA Class-I or Class II Surface Expression and Improve Evasion of Allorejection

Experiments were undertaken to ablate HLA class-I surface expression to overcome recognition of allogeneic immune cells by the recipient's T cells and/or NK cells. To do so, base editor systems (e.g., ABE8.20 and rBE mRNA600 in combination with gRNAs) and/or nuclease systems (e.g., Cas12b in combination with gRNAs) were designed to target 1) genes implicated in transcriptional activation of HLA class-I expression (CITA; NLRC5), 2) genes involved in proper assembly of mature HLA class-I molecules (e.g., TAP1, TAP2, ERp57 (PDIA3), and TAPBP (Tapasin)), and 3) CIITA involved in HLA class-II surface expression. Moreover, to enable HLA class-I deficient immune cells to evade recipient NK cell rejection, base editor and/or nuclease systems were designed to disrupt genes encoding proteins that bind NK cell activation receptors, including CD155, Nectin-2, CD48, MICA, MICB, and ULBP (see FIG. 19). Guide RNAs (see Table 1A) were designed for each gene to direct a CBE (rBE4 mRNA600), ABE (ABE8.20), and/or nuclease (Cas12b) to disrupt protein expression from the target genes (e.g., by altering the translational start site, by altering conserved splicing motifs, or by introducing premature stop codons).

The guide RNAs used included guide RNAs targeting multiple MICA, MICB, and/or ULBP alleles. The guide RNAs targeting multiple alleles of a target gene(s) were prepared based upon nucleotide sequence alignments of the sequences corresponding to multiple alleles of the target gene(s).

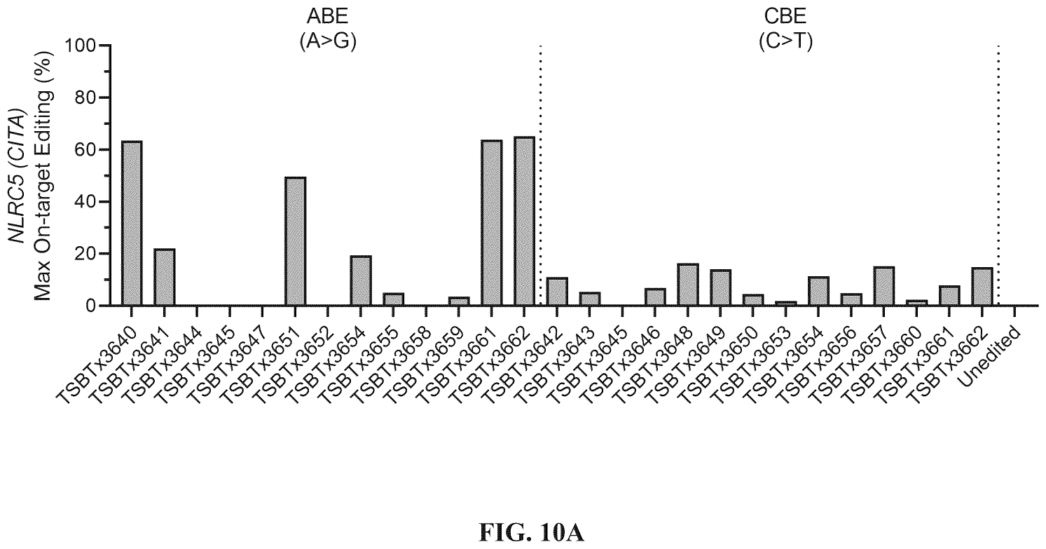

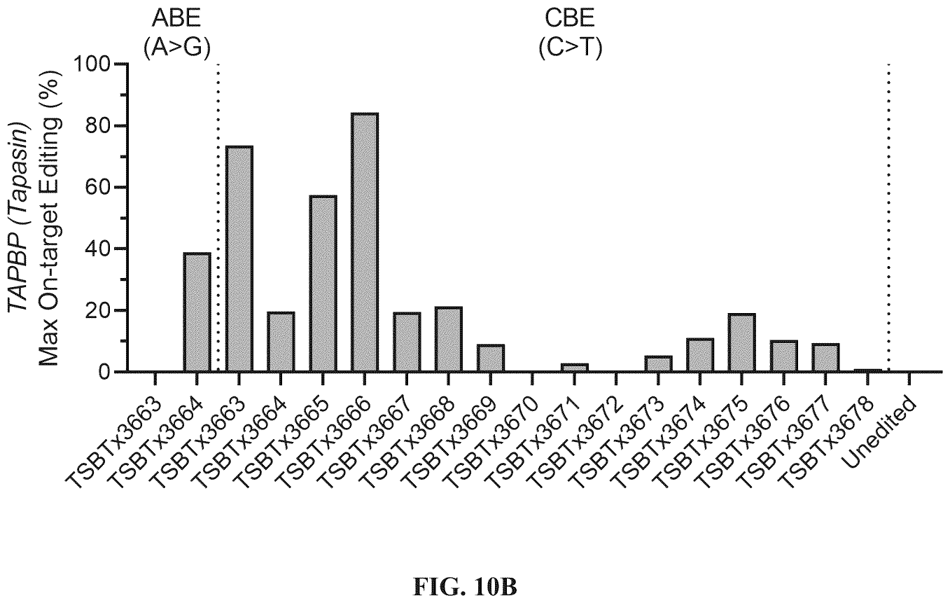

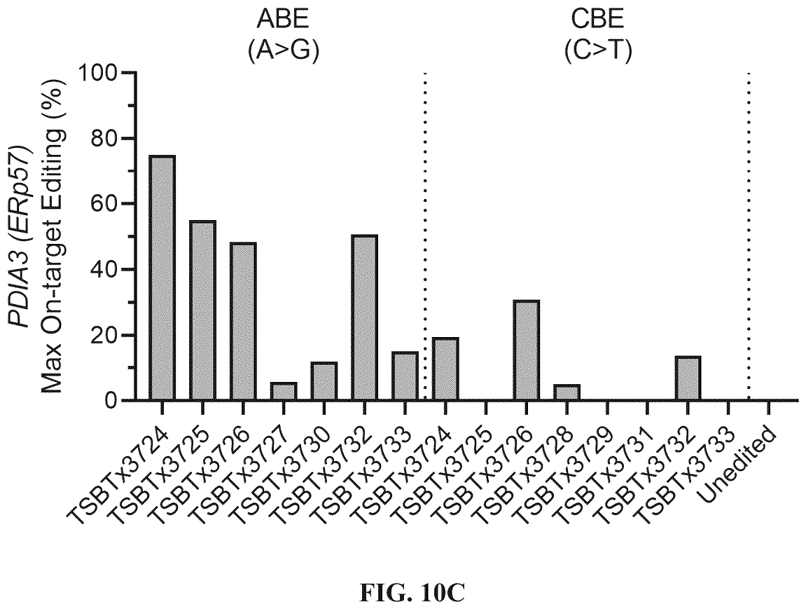

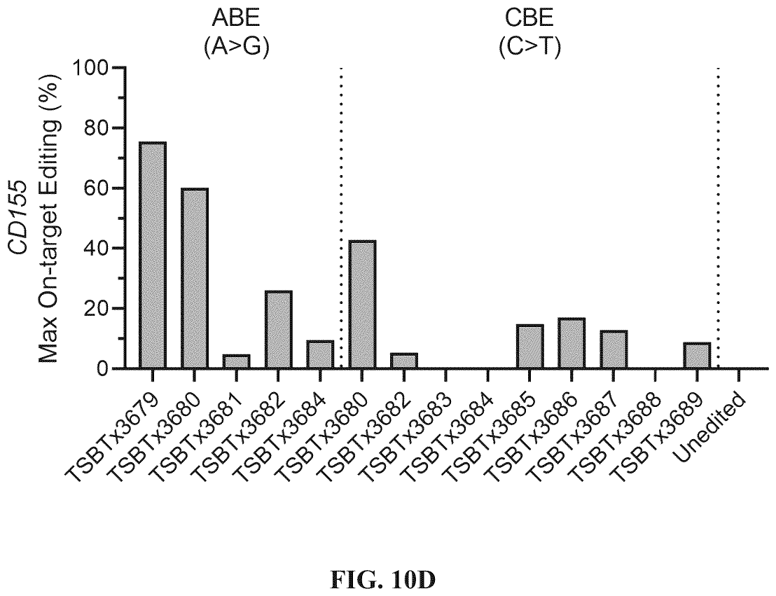

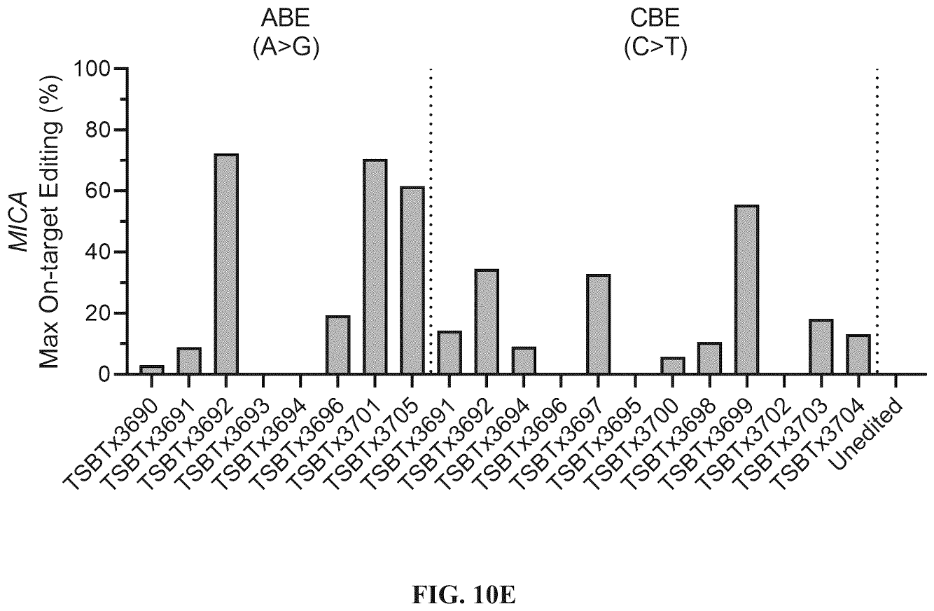

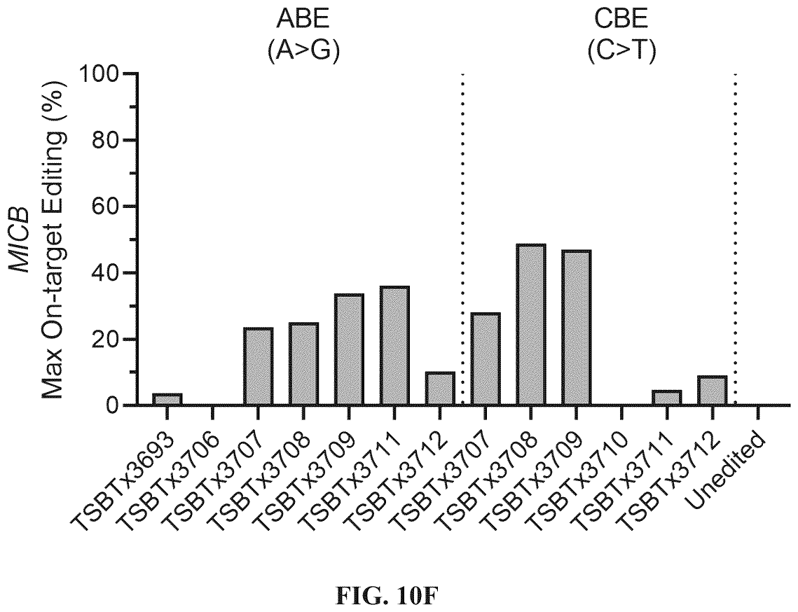

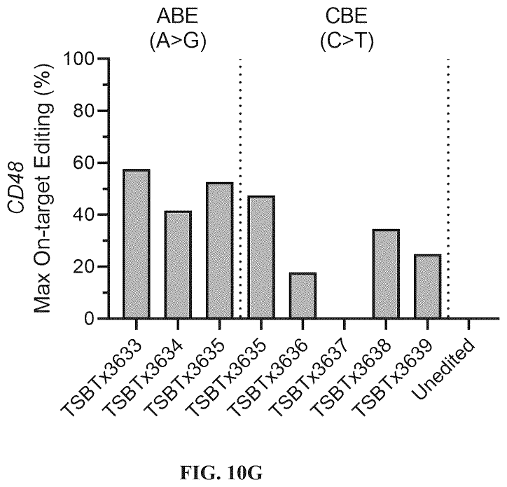

Guide RNAs were screened for use with ABE8.20 and rBE4 mRNA600 in primary human T cells 2 days post-activation by electroporation of the cells with mRNA encoding the appropriate editor and guide RNA. After 3 days, the cells were harvested and next-generation sequencing (NGS) was performed to determine editing efficiency of the target sequence at the genomic level (see FIGS. 10A-10G). In some instances, flow cytometry was performed to detect protein expression.

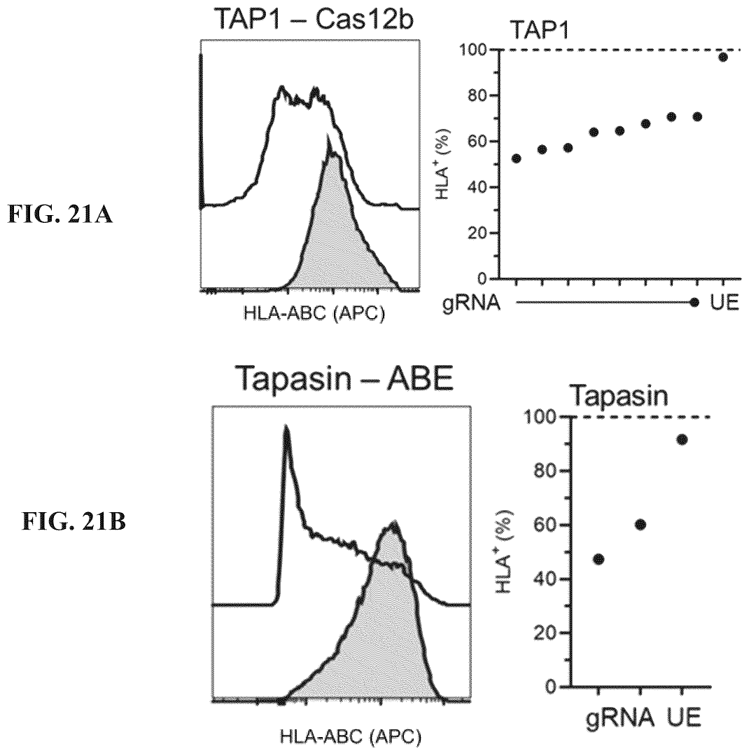

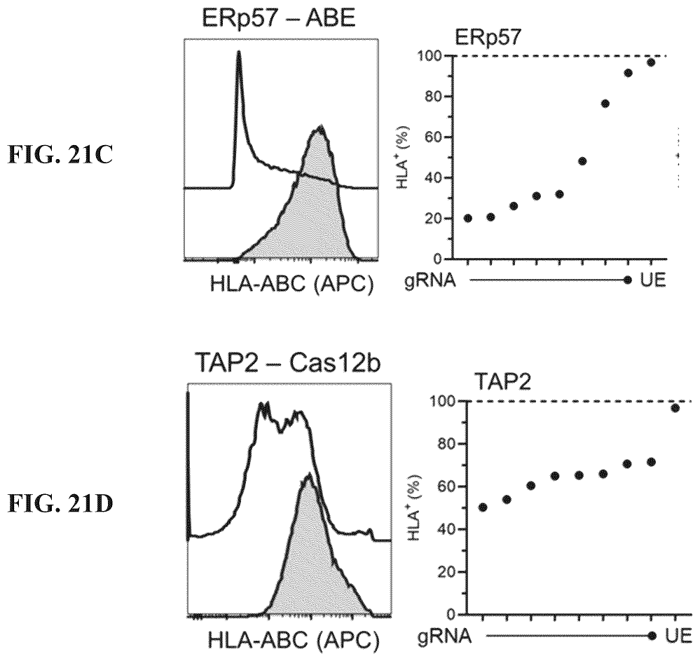

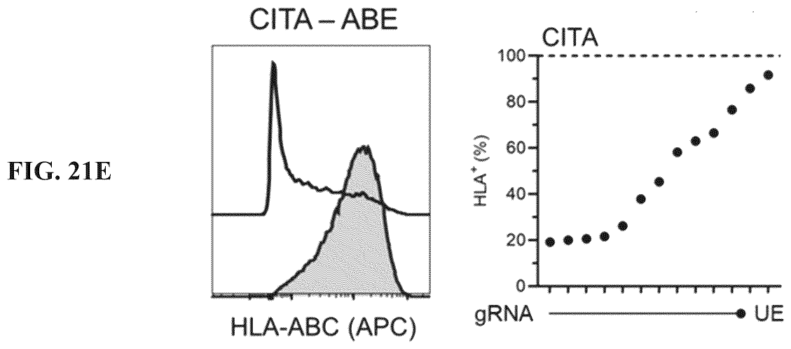

Base editing in T cells of TAP1 or TAPBP using gRNAs (see Table 1A) in combination with Cas12b or base editing of Tapasin, ERp571PDIA3, or CITA using gRNAs (see Table 1A) in combination with an ABE resulted in reduced expression of HLA-ABC, as measured by flow cytometry (see FIGS. 21A-21E). These results demonstrate that knock-out of peptide loading genes reduced HLA class-I surface expression.

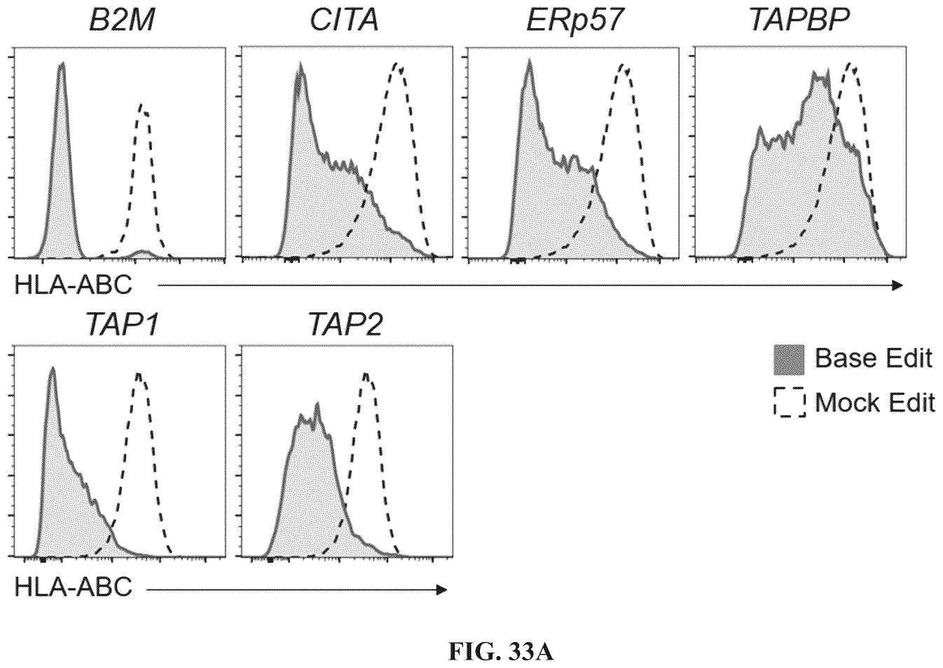

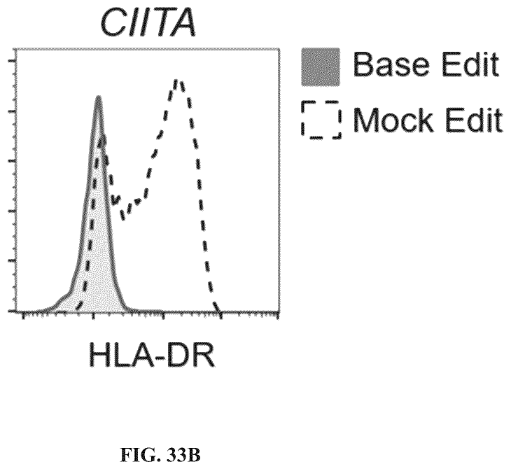

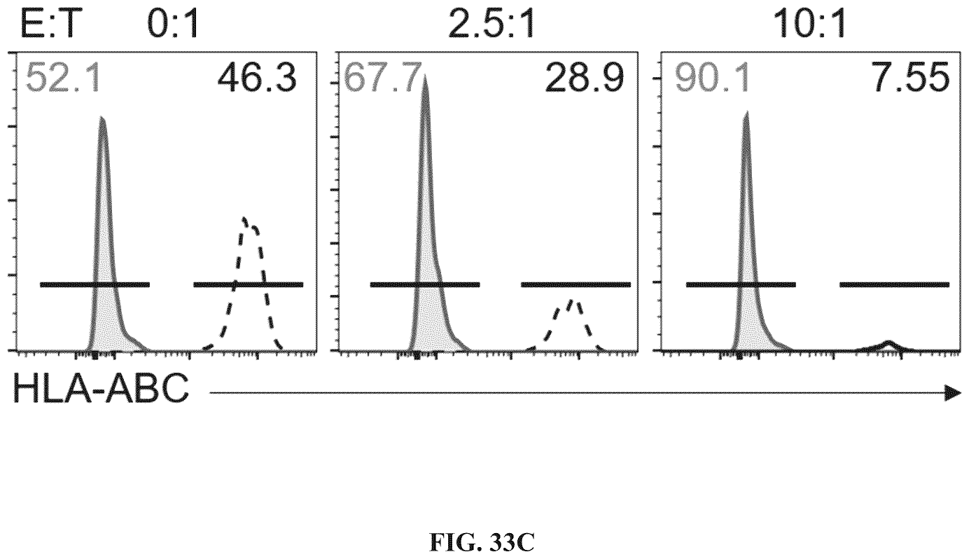

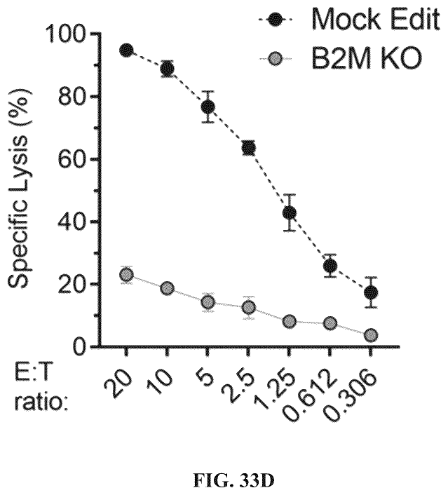

Reduction in polypeptide expression through base editing of β2M, CITA, ERp57, TAPBP, TAP1, TAP2, and CIITA using gRNAs (see Table 1A) in combination with rBE4 or ABE8.20m was confirmed using flow cytometry (see FIGS. 33A and 33B).

An in vitro T cell Mixed Leukocyte Reaction (MLR) using target T cells (Mock Edit and β2M knock-out cells prepared using the methods and base editor systems (see Table 1A) provided herein) co-cultured with HLA mismatched effector cells was completed. The MLR demonstrated that knockout of β2M was associated with evasion of T cell-mediated allorejection in vitro (see FIGS. 33C and 33D).

### Example 10: HLA-E and HLA-G Single-Chain Trimers and Dimers

HLA-E and HLA-G constructs were designed for overexpression in immune cells to allow for the immune cells to evade NK cells. The HLA-E and HLA-G constructs were designed to be suitable for over-expression in the immune cells. The HLA-E and HLA-G constructs bind to the NKG2A inhibitory receptor of natural killer (NK) cells, thereby inhibiting the NK cells and preventing lysis of the immune cells thereby. The constructs were designed to allow immune cells expressing the constructs to resist allogeneic rejection mechanisms of a host subject.

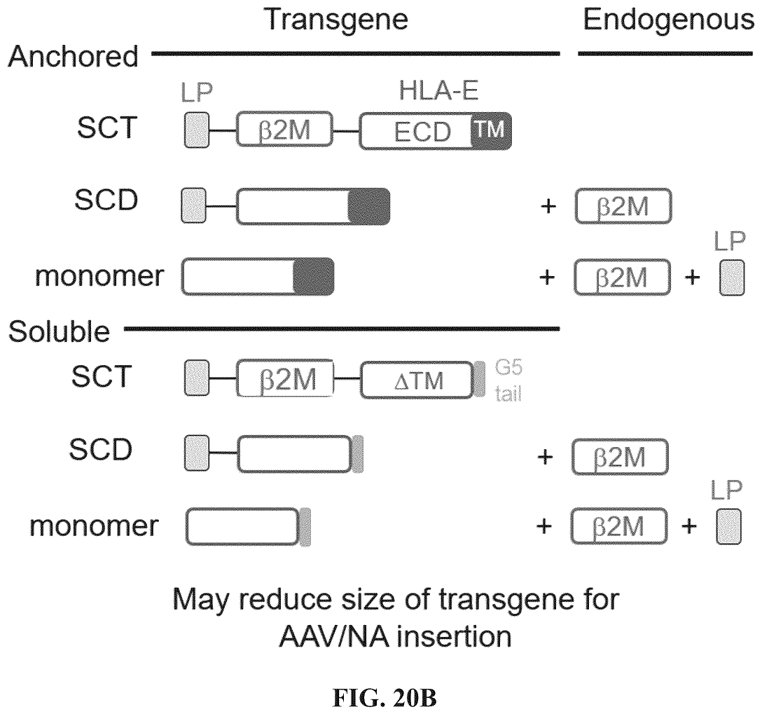

The HLA-E and HLA-G constructs included secreted single-chain trimers (see Table 19 below), secreted single-chain dimers (see Table 19 below), and a membrane-bound single-chain HLA-E trimer (see sequence provided below). A schematic diagram of a representative secreted single-chain trimer is provided in FIG. 11 and schematic diagrams of the single-chain trimer and other constructs are provided in FIGS. 20A and 20B. The single-chain trimers contained a signal peptide, a loading peptide, Gly/Ser (GS) linkers, a β2M domain, and an HLA domain. The single-chain dimers were similar to the single-chain trimers in construction, but differed in that the single-chain dimers did not include any β2M domain.

TABLE 19
Secreted HLA-E and HLA-G constructs
Construct: BTx_CM118

| | |
|---|---|
| HLA-G5 | MVVMAPRTLFLLLSGALTLTETWAGSHS |
| isoform | MRYFSAAVSRPGRGEPRFIAMGYVDDTQ |
| | FVRFDSDSACPRMEPRAPWVEQEGPEYW |
| | EEETRNTKAHAQTDRMNLQTLRGYYNQS |
| | EASSHTLQWMIGCDLGSDGRLLRGYEQY |
| | AYDGKDYLALNEDLRSWTAADTAAQISK |
| | RKCEAANVAEQRRAYLEGTCVEWLHRYL |
| | ENGKEMLQRADPPKTHVTHHPVFDYEAT |
| | LRCWALGFYPAEIILTWQRDGEDQTQDV |
| | ELVETRPAGDGTFQKWAAVVVPSGEEQR |
| | YTCHVQHEGLPEPLMLRWSKEGDGGIMS |
| | VRESRSLSEDL |
| | (SEQ ID NO: 1012) |

Construct: BTx_CM119

| | |
|---|---|
| HLA-G5 + | MYRMQLLSCIALSLALVTNSGSHSMRYF |
| IL-2 | SAAVSRPGRGEPRFIAMGYVDDTQFVRF |
| signal | DSDSACPRMEPRAPWVEQEGPEYWEEET |
| peptide | RNTKAHAQTDRMNLQTLRGYYNQSEASS |
| | HTLQWMIGCDLGSDGRLLRGYEQYAYDG |
| | KDYLALNEDLRSWTAADTAAQISKRKCE |
| | AANVAEQRRAYLEGTCVEWLHRYLENGK |
| | EMLQRADPPKTHVTHHPVFDYEATLRCW |
| | ALGFYPAEIILTWQRDGEDQTQDVELVE |
| | TRPAGDGTFQKWAAVVVPSGEEQRYTCH |
| | VQHEGLPEPLMLRWSKEGDGGIMSVRES |
| | RSLSEDL |
| | (SEQ ID NO: 1013) |

Construct: BTx_CM120

| | |
|---|---|
| HLA-G5 | MYRMQLLSCIALSLALVTNSIQRTPKIQ |
| Single | VYSRHPAENGKSNFLNCYVSGFHPSDIE |
| chain | VDLLKNGERIEKVEHSDLSFSKDWSFYL |
| trimer + | LYYTEFTPTEKDEYACRVNHVTLSQPKI |
| IL-2 | VKWDRDMGGGGSGGGGSGGGGSRIIPRH |
| signal | LQLGGGGSGGGGSGGGGSGGGGSGSHSM |
| peptide | RYFSAAVSRPGRGEPRFIAMGYVDDTQF |
| | VRFDSDSACPRMEPRAPWVEQEGPEYWE |
| | EETRNTKAHAQTDRMNLQTLRGYYNQSE |
| | ASSHTLQWMIGCDLGSDGRLLRGYEQYA |
| | YDGKDYLALNEDLRSWTAADTAAQISKR |
| | KCEAANVAEQRRAYLEGTCVEWLHRYLE |
| | NGKEMLQRADPPKTHVTHHPVFDYEATL |
| | RCWALGFYPAEIILTWQRDGEDQTQDVE |
| | LVETRPAGDGTFQKWAAVVVPSGEEQRY |
| | TCHVQHEGLPEPLMLRWSKEGDGGIMSV |
| | RESRSLSEDL |
| | (SEQ ID NO: 1014) |

Construct: BTx_CM193

| | |
|---|---|
| HLA-E | MSRSVALAVLALLSLSGLEAVMAPRTLF |
| (ΔTM) | LGGGGSGGGGSGGGGSIQRTPKIQVYSR |
| Single | HPAENGKSNFLNCYVSGFHPSDIEVDLL |
| chain | KNGERIEKVEHSDLSFSKDWSFYLLYYT |
| trimer + | EFTPTEKDEYACRVNHVTLSQPKIVKWD |
| HLA-G5 | RDMGGGGSGGGGSGGGGSGGGGSGSHSL |
| intron | KYFHTSVSRPGRGEPRFISVGYVDDTQF |
| tail | VRFDNDAASPRMVPRAPWMEQEGSEYWD |
| | RETRSARDTAQIFRVNLRTLRGYYNQSE |
| | AGSHTLQWMHGCELGPDGRFLRGYEQFA |
| | YDGKDYLTLNEDLRSWTAVDTAAQISEQ |
| | KSNDASEAEHQRAYLEDTCVEWLHKYLE |
| | KGKETLLHLEPPKTHVTHHPISDHEATL |
| | RCWALGFYPAEITLTWQQDGEGHTQDTE |
| | LVETRPAGDGTFQKWAAVVVPSGEEQRY |
| | TCHVQHEGLPEPVTLRWSKEGDGGIMSV |
| | RESRSLSEDL |
| | (SEQ ID NO: 1015) |

Construct: BTx_CM211

| | |
|---|---|
| HLA-E | MSRSVALAVLALLSLSGLEAVMAPRTLF |
| (ΔTM) | LGGSGGGASGGGSHSLKYFHTSVSRPGR |
| β2M | GEPRFISVGYVDDTQFVRFDNDAASPRM |
| (C- | VPRAPWMEQEGSEYWDRETRSARDTAQI |
| term) | FRVNLRTLRGYYNQSEAGSHTLQWMHGC |
| Single | ELGPDGRFLRGYEQFAYDGKDYLTLNED |
| chain | LRSWTAVDTAAQISEQKSNDASEAEHQR |
| trimer | AYLEDTCVEWLHKYLEKGKETLLHLEPP |
| | KTHVTHHPISDHEATLRCWALGFYPAEI |
| | TLTWQQDGEGHTQDTELVETRPAGDGTF |
| | QKWAAVVVPSGEEQRYTCHVQHEGLPEP |
| | VTLRWGGGGSGGGGSGGGGSIQRTPKIQ |
| | VYSRHPAENGKSNFLNCYVSGFHPSDIE |
| | VDLLKNGERIEKVEHSDLSFSKDWSFYL |
| | LYYTEFTPTEKDEYACRVNHVTLSQPKI |
| | VKWDRDM |
| | (SEQ ID NO: 1016) |

Construct: BTx_CM212

| | |
|---|---|
| HLA-E | MSRSVALAVLALLSLSGLEAVMAPRTLF |
| (ΔTM) | LGGSGGGASGGGSHSLKYFHTSVSRPGR |
| Single | GEPRFISVGYVDDTQFVRFDNDAASPRM |
| chain | VPRAPWMEQEGSEYWDRETRSARDTAQI |
| dimer | FRVNLRTLRGYYNQSEAGSHTLQWMHGC |
| + | ELGPDGRFLRGYEQFAYDGKDYLTLNED |
| HLA-G5 | LRSWTAVDTAAQISEQKSNDASEAEHQR |
| intron | AYLEDTCVEWLHKYLEKGKETLLHLEPP |
| tail | KTHVTHHPISDHEATLRCWALGFYPAEI |
| | TLTWQQDGEGHTQDTELVETRPAGDGTF |
| | QKWAAVVVPSGEEQRYTCHVQHEGLPEP |
| | VTLRWSKEGDGGIMSVRESRSLSEDL |
| | (SEQ ID NO: 1017) |

Construct: BTx_CM213

| | |
|---|---|
| HLA-E | MSRSVALAVLALLSLSGLEAVMAPRTLF |
| (ΔTM) | LGGSGGGASGGGSHSLKYFHTSVSRPGR |
| Single | GEPRFISVGYVDDTQFVRFDNDAASPRM |
| chain | VPRAPWMEQEGSEYWDRETRSARDTAQI |
| dimer | FRVNLRTLRGYYNQSEAGSHTLQWMHGC |
| | ELGPDGRFLRGYEQFAYDGKDYLTLNED |
| | LRSWTAVDTAAQISEQKSNDASEAEHQR |
| | AYLEDTCVEWLHKYLEKGKETLLHLEPP |
| | KTHVTHHPISDHEATLRCWALGFYPAEI |
| | TLTWQQDGEGHTQDTELVETRPAGDGTF |
| | QKWAAVVVPSGEEQRYTCHVQHEGLPEP |
| | VTLRW |
| | (SEQ ID NO: 1018) |

In Table 19, the HLA domain is indicated by underlined text, linkers are indicated by bold text, signal peptides (e.g., the signal peptide of HLA-G, IL-2, or β2M) are indicated by italicized text, loading peptide are indicated by bold underlined text, an HLA-G5 intron tail is indicated by double-underlined text, and the β2M domain is indicated by bold, italic, underlined text.

Provided below is the sequence of the membrane-bound single-chain trimer that was designed (BTx_CM188):

(SEQ ID NO: 1019)
MSRSVALAVLALLSLSGLEAVMAPRTLFLGGGG
SGGGGSGGGGSIQRTPKIQVYSRHPAENGKSNF
LNCYVSGFHPSDIEVDLLKNGERIEKVEHSDLS
FSKDWSFYLLYYTEFTPTEKDEYACRVNHVTLS
QPKIVKWDRDMGGGGSGGGGSGGGGSGGGGSGS
HSLKYFHTSVSRPGRGEPRFISVGYVDDTQFVR
FDNDAASPRMVPRAPWMEQEGSEYWDRETRSAR
DTAQIFRVNLRTLRGYYNQSEAGSHTLQWMHGC
ELGPDGRFLRGYEQFAYDGKDYLTLNEDLRSWT
AVDTAAQISEQKSNDASEAEHQRAYLEDTCVEW
LHKYLEKGKETLLHLEPPKTHVTHHPISDHEAT
LRCWALGFYPAEITLTWQQDGEGHTQDTELVET
RPAGDGTFQKWAAVVVPSGEEQRYTCHVQHEGL
PEPVTLRWKPASQPTIPIMALIVLGGVAGLLLF
IGLGIFFCVRC.

In the above sequence, which corresponds to an HLA-E SCT (CD4TM) polypeptide, the HLA-E heavy chain domain is indicated by underlined text, linkers are indicated by bold text, the signal peptide is indicated by italicized text, the loading peptide (alternatively, “peptide for presentation”) is indicated by bold underlined text, the β2M domain is indicated by bold, italic, underlined text, the CD4 transmembrane domain is indicated by plain text, and the CD4 truncated intracellular domain is indicated by double-underlined text.

### Example 11: Detection of HLA-E Single-Chain Dimers and Trimers Secreted by CAR-T Cells

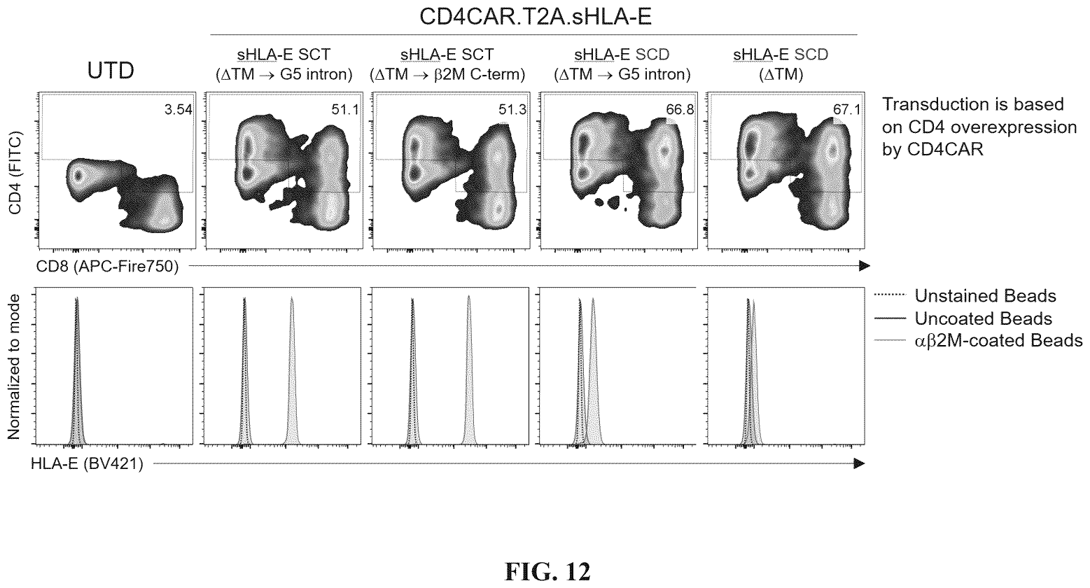

Secretion of the HLA-E single-chain dimer and trimer constructs (BTx_CM193, BTx_CM211, BTx_CM212, and BTx_CM213) was confirmed in anti-CD4 CAR-T cells (see FIG. 12). The anti-CD4 CAR-T cells were transduced with polynucleotides encoding each of the constructs. Expression of the constructs was under the control of the promoter elongation factor-1 alpha. The anti-CD4 CAR-T cells did not express a functional β2M polypeptide.

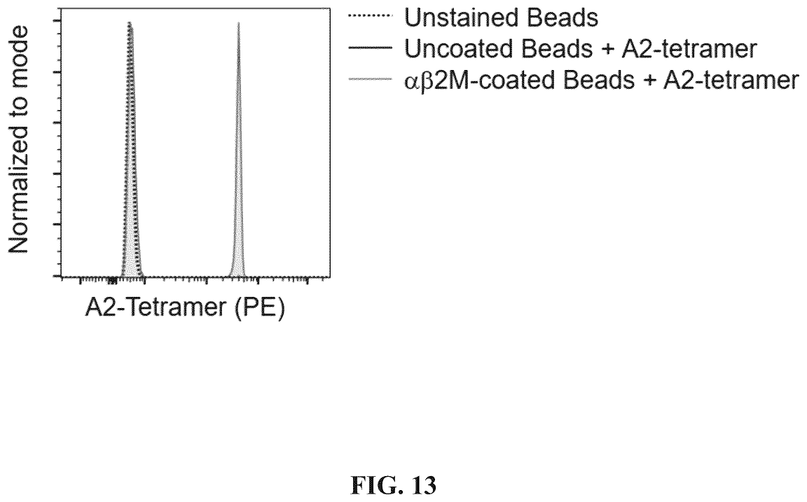

Secretion of the soluble HLA-E constructs was detected in culture supernatant using M-280 streptavidin Dynabeads conjugated to an anti-β2M antibody (αβ2M-coated beads). As a positive control, beads binding PE-labeled HLA-A2 tetramers were detected using flow cytometry (see FIG. 13). Cell-free supernatant gathered from the cells (UTD and cells expressing the constructs) was incubated with the M-280 streptavidin Dynabeads conjugated to an anti-β2M antibody and, as a negative control, M-280 streptavidin Dynabeads that were not conjugated to any antibody (uncoated beads). The incubated beads were then stained with anti-HLA-E antibody. As a further control, some incubated beads were not stained with the anti-HLA-E antibody (unstained beads). As shown in FIG. 12, all of the constructs were successfully secreted by the anti-CD4 CAR-T cells.

### Example 12: The HLA-E Single-Chain Trimer Constructs Protected Cells from Lysis by Natural Killer (NK) Cells

Experiments were undertaken to confirm that the expression of the HLA-E single-chain trimer constructs of Table 19 facilitated evasion of cell lysis by natural killer cells.

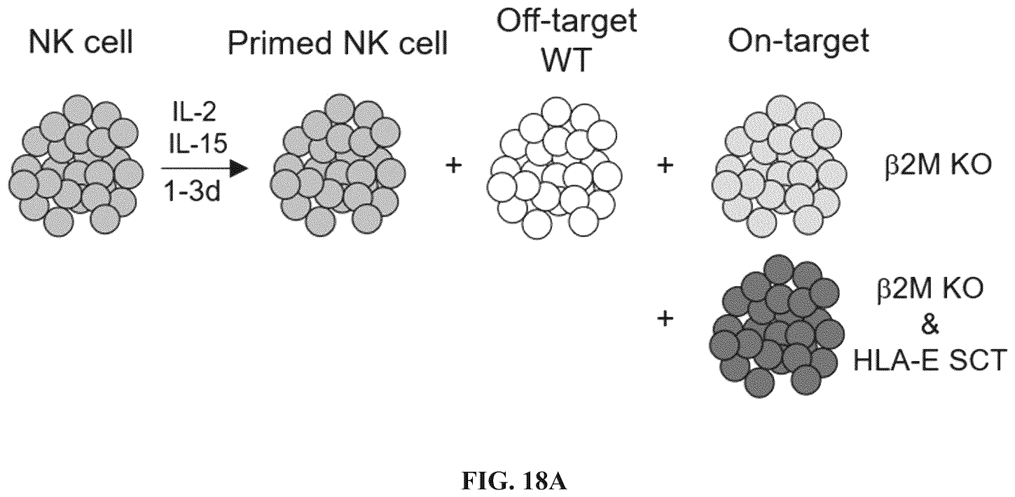

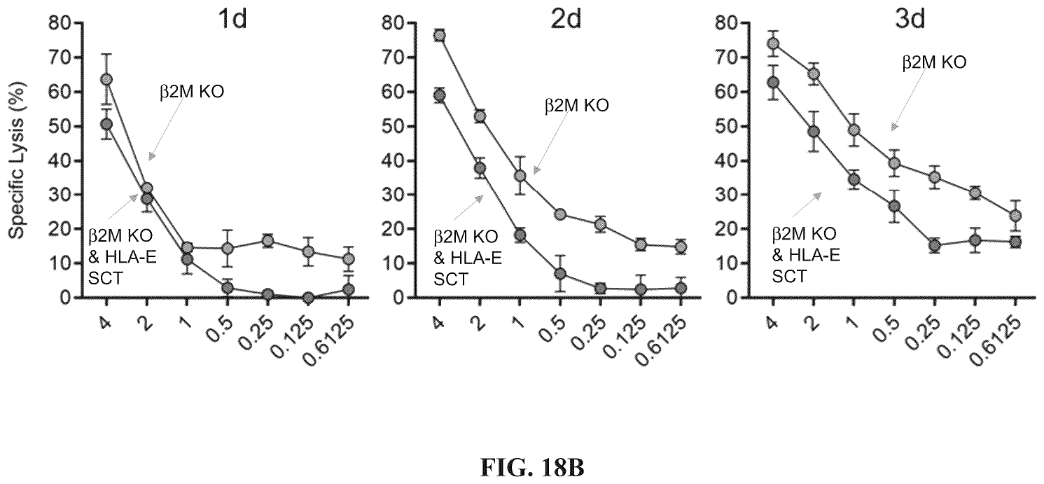

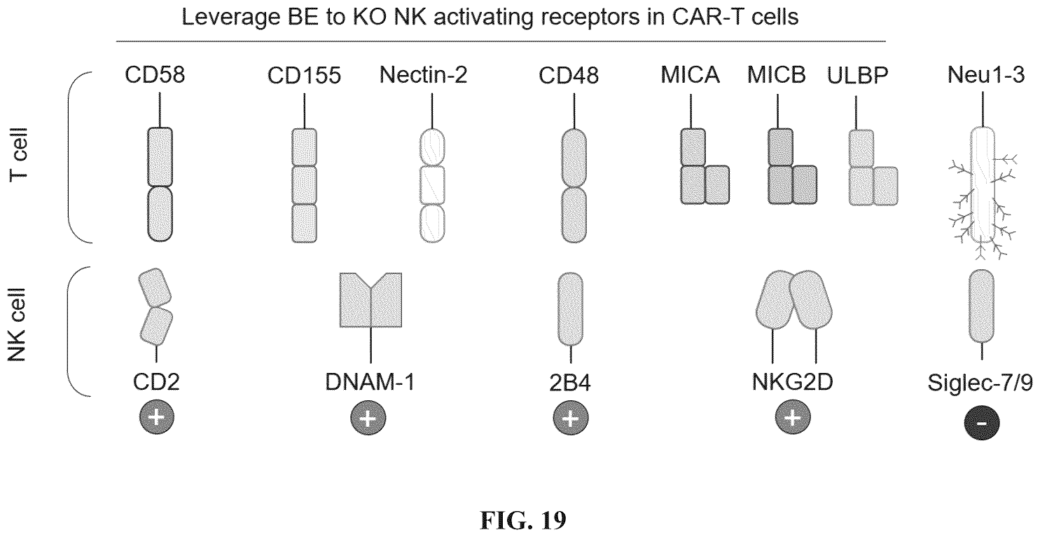

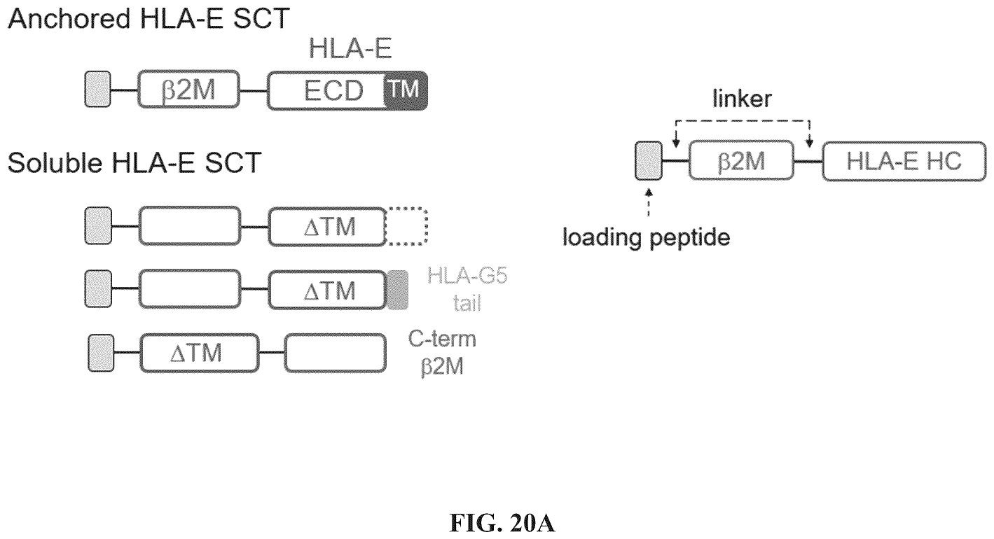

First, a mixed leukocyte reaction was prepared (see FIG. 18A) where natural killer cells were cultured in the presence of IL-2 and IL-15 for 1-3 days to yield primed NK cells. The primed NK cells were then mixed with off-target wild-type (WT; unedited) cells and either on-target β2M knock-out cells or on-target β2M knock-out T cells that also expressed the membrane-bound (“anchored”) HLA-E single-chain trimer (SCT) BTx_CM188 (see Example 10). The β2M gene was knocked out according to methods described herein using ABE8.20m and the guide RNA TSBTx760 (see Table 1A). Specific lysis was measured at 1 day, 2 days, and 3 days (FIG. 18B). Expression of the membrane-anchored HLA-E single-chain trimer conferred protection (from about 20% to about 30%) against in vitro NK cell mediated killing in vitro.

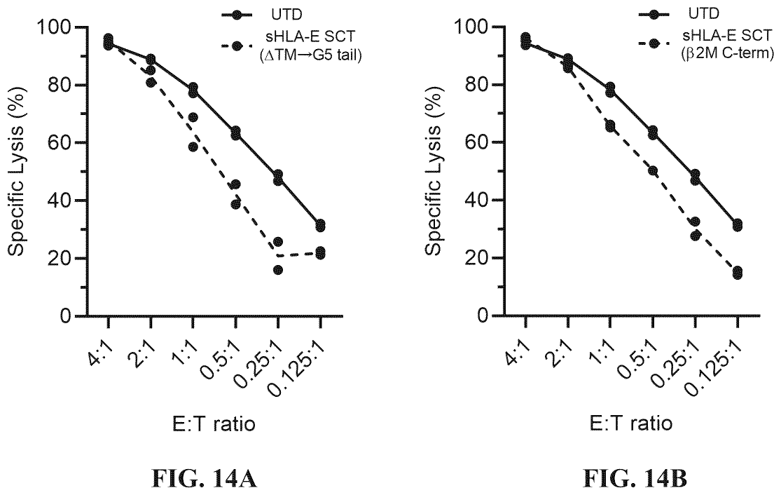

β2M knock-out CAR-T cells expressing the soluble HLA-E single-chain trimers (sHLA-SCTs) BTxCM193 and BTx_CM211 of Table 19 were exposed to natural killer cells. It was demonstrated that expression of the constructs was associated with inhibition of lysis of the CAR-T cells by the natural killer cells (FIGS. 14A and 14B). Therefore, secretion of the soluble HLA isoforms suppressed NK cells and reduced immunogenicity.

### Example 13: Evaluation of the Efficacy of the HLA-E and HLA-G Single-Chain Dimer and/or Trimer Constructs in the Context of BLT (Bone Marrow, Liver, and Thymus) Humanized Mice

Experiments are undertaken in vivo to confirm that the expression of the HLA-E and HLA-G single-chain dimer and trimer constructs of Table 19 or the single-chain membrane-bound trimer construct HLA-E SCT (CD4TM) (SEQ ID NO: 1019) facilitates evasion of cell lysis by natural killer cells in NS6 BLT (Bone marrow Liver+Thymus humanized) mice.

The NS6 BLT mice are injected with CAR-T cells expressing the constructs. At 24 hours and every 7 days, retro-orbital puncture is performed to obtain blood, which is then analyzed. Necrotyzed mice are also analyzed. The experiments demonstrate that expression of the constructs is associated with increased persistence of the CAR-T cells in the mice.

### Example 14: Evaluation of the Impact of Beta 2 Microglobulin (P2M) Knock-Out on T Cell and NK Cell Mediated Killing of Edited T Cells

Experiments were undertaken to determine the impact of β2M knock-out on T cell and NK cell mediated killing of edited cells.

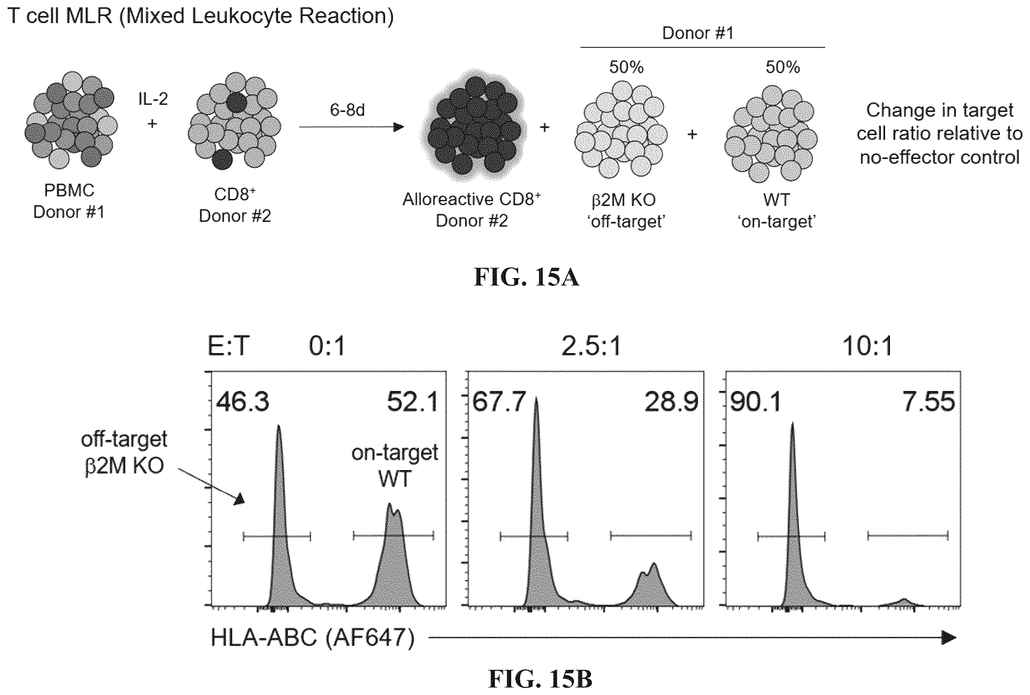

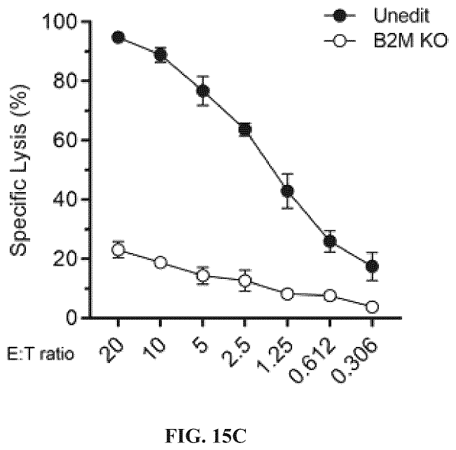

First, a mixed leukocyte reaction was prepared (see FIG. 15A) where peripheral blood mononuclear cells (PBMCs) from a Donor #1 were cocultured with CD8+ cells from Donor 2 for from 6-8 days to prepare alloreactive CD8+ Donor #2 cells. The alloreactive CD8+ Donor #2 cells were then mixed with a 50:50 mixture of β2M knock-out T cells (off-target cells) and unedited cells (WT, on-target cells) at different ratios of effector to target cells (E:T). Cell lysis was measured by flow cytometry (see FIGS. 15B and 15C). The β2M gene was knocked out according to methods described herein using ABE8.20m and the guide RNA TSBTx760 (see Table 1A). β2M knock-out T cells were protected from T cell mediated killing (see FIGS. 15B and 15C).

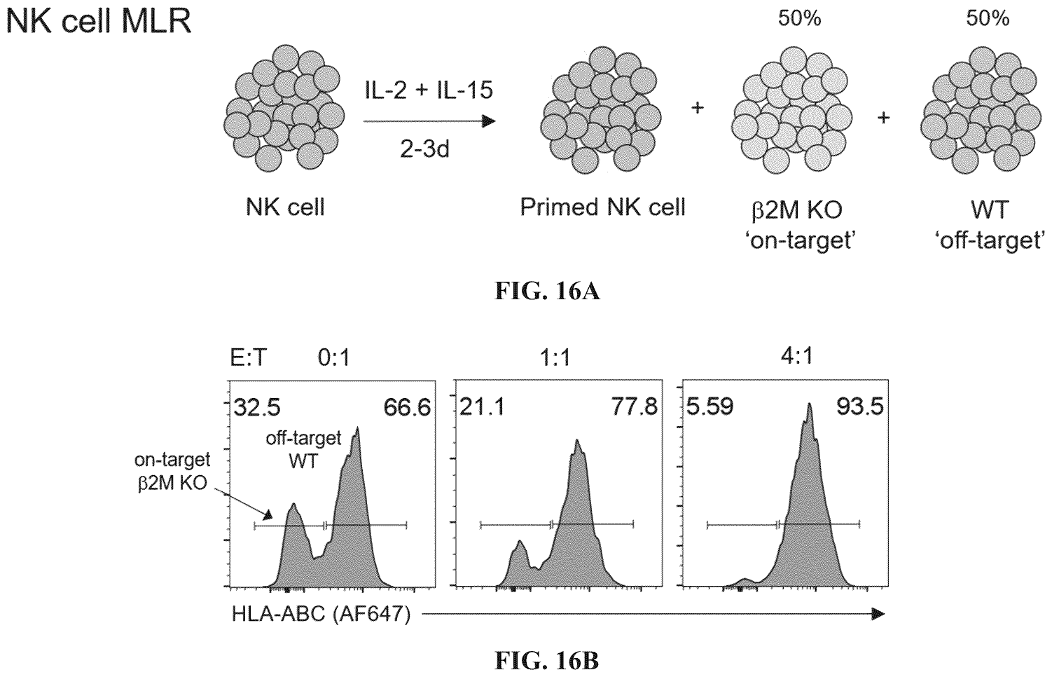

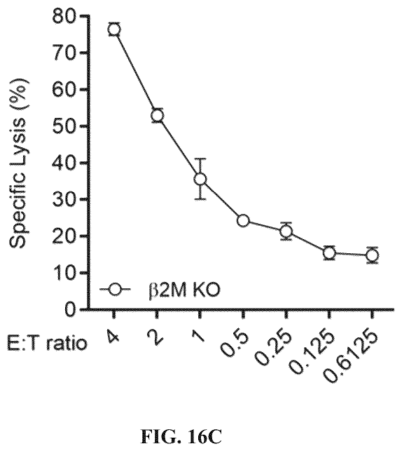

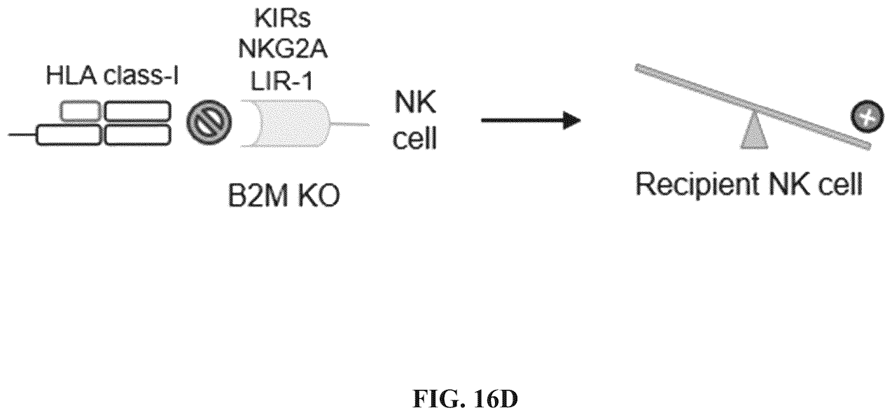

Next, a mixed leukocyte reaction was prepared (see FIG. 16A) where natural killer cells were cultured in the presence of IL-2 and IL-15 for 2-3 days to yield primed NK cells. The primed NK cells were then mixed with a 50-50 mixture of the β2M knock-out T cells (on-target cells) and unedited cells (WT, off-target cells) at different ratios of effector to target cells (E:T). Cell lysis was measured by flow cytometry (see FIGS. 16B and 16C). β2M knock-out T cells showed increased susceptibility to in vitro NK cell-mediated killing (see FIGS. 16B-16D).

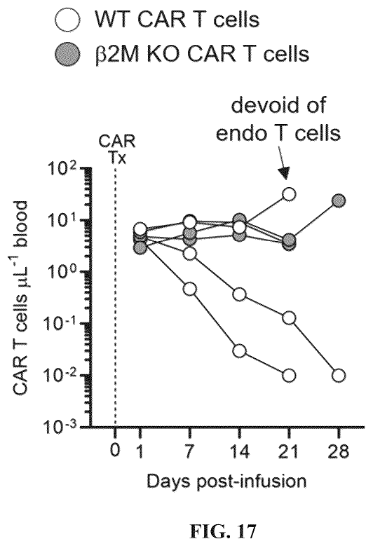

Finally, allorejection of the β2M knock-out T cells was evaluated in the BLT humanized mouse model. The BLT humanized mouse model recapitulates key features of allorejection, such as rejection of HLA mismatched T cells (see FIGS. 5A-5C and Example 7). The β2M knock-out T cells resisted allorejection by the BLT mice (FIG. 17), but were susceptible to rejection by NK cells in the BLT mice (see FIG. 8E and Example 7).

Therefore, β2M knock-out conferred protection against T cell allorejection, but increased susceptibility to NK cell lysis.

### Example 15: Screening of sgRNA Libraries to Identify sgRNAs Effective for Targeting a CBE for Base Editing B2M, TAP1, TAP2, TAPBP, or TAPBPL

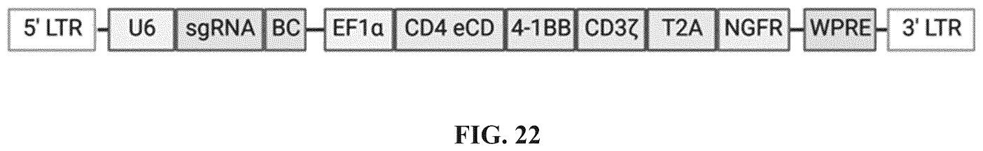

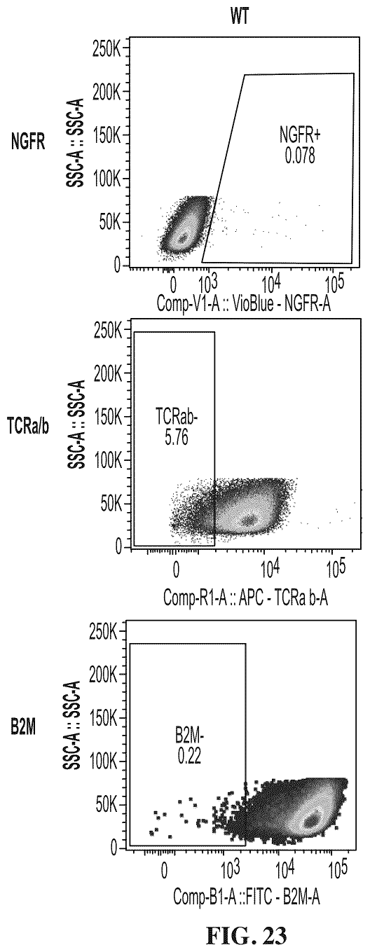

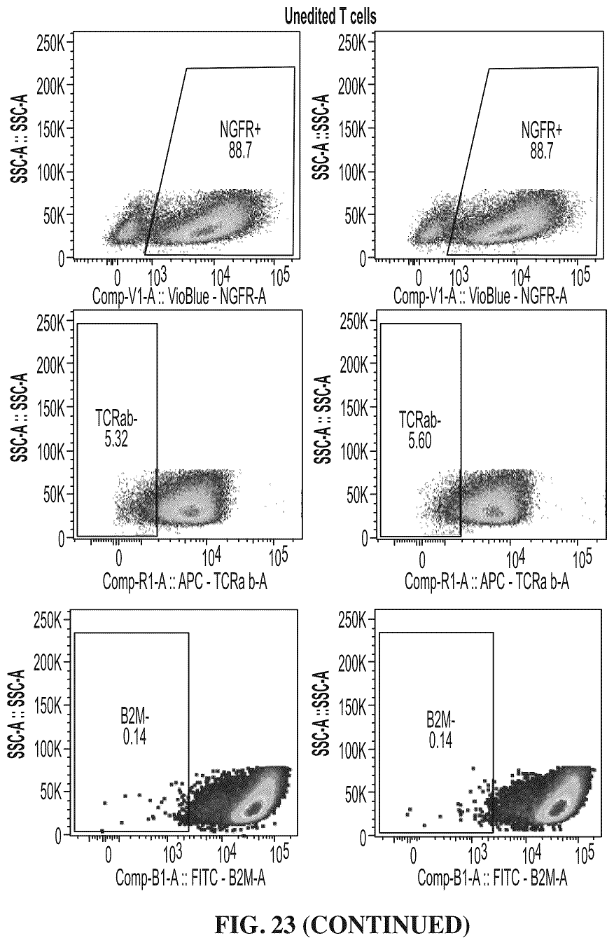

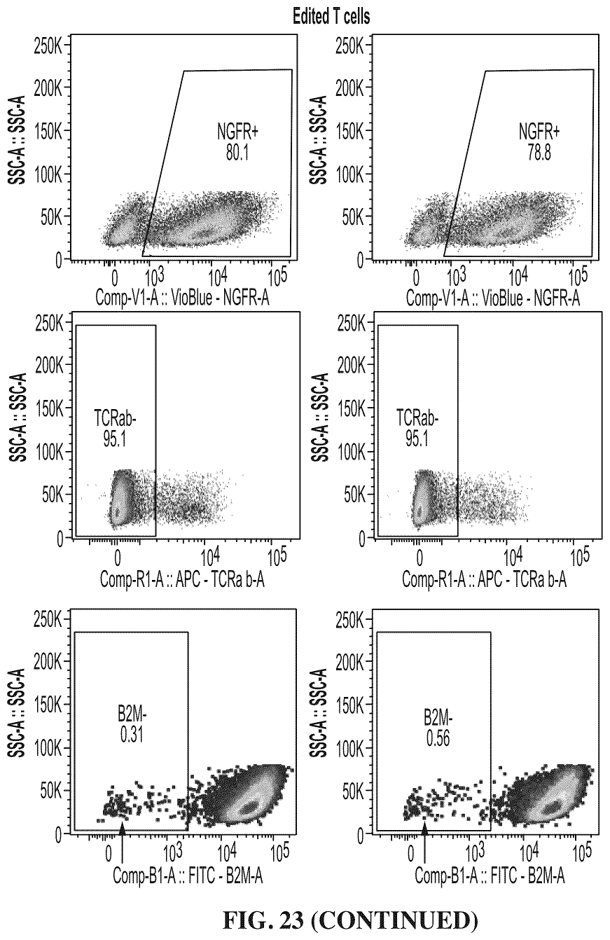

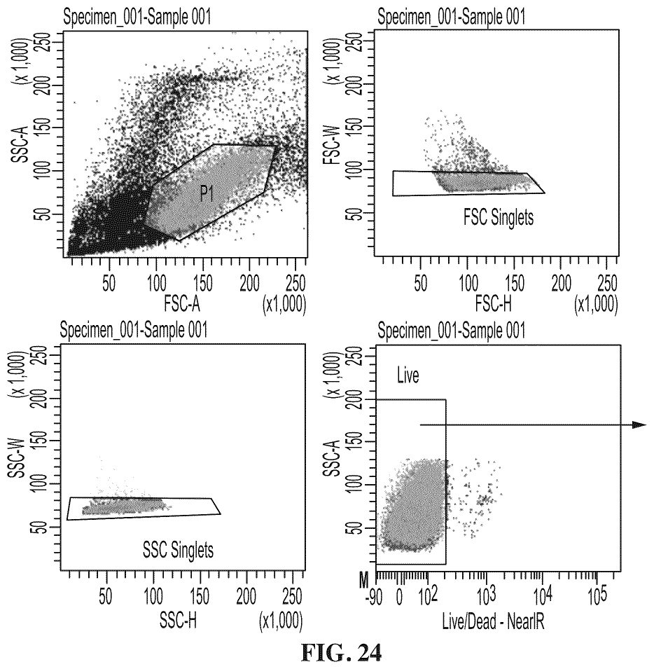

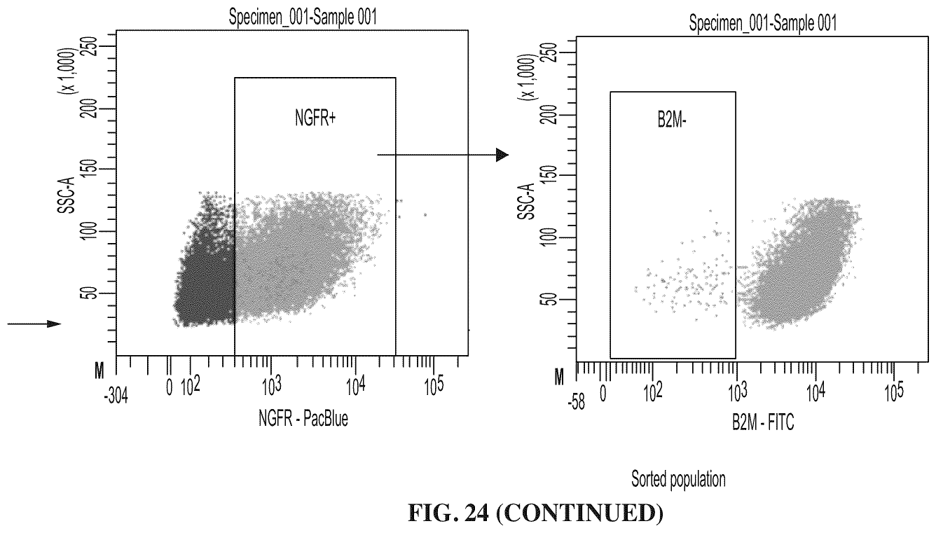

Experiments were undertaken to screen libraries of guide RNA sequences to identify those that can be used effectively to target a CBE to base edit B2M, TAP1, TAP2, TAPBP, and/or TAPBPL to result in reduced levels of B2M/MHL I expression in T cells to improve allogeneic persistence in a subject. The design of the expression constructs used in the library are shown in FIG. 22. Libraries were prepared comprising about 2845 sgRNA sequences including 113 essential gene guides and 120 non-targeting guides. T cells expressing a CBE were transfected with the library of sgRNA sequences using lentiviral vectors. The transfected cells were then sorted to identify B2M− T cells (see FIGS. 23 and 24). Approximately 200M cells were sorted to yield a sorted population of about 400 k sorted cells.

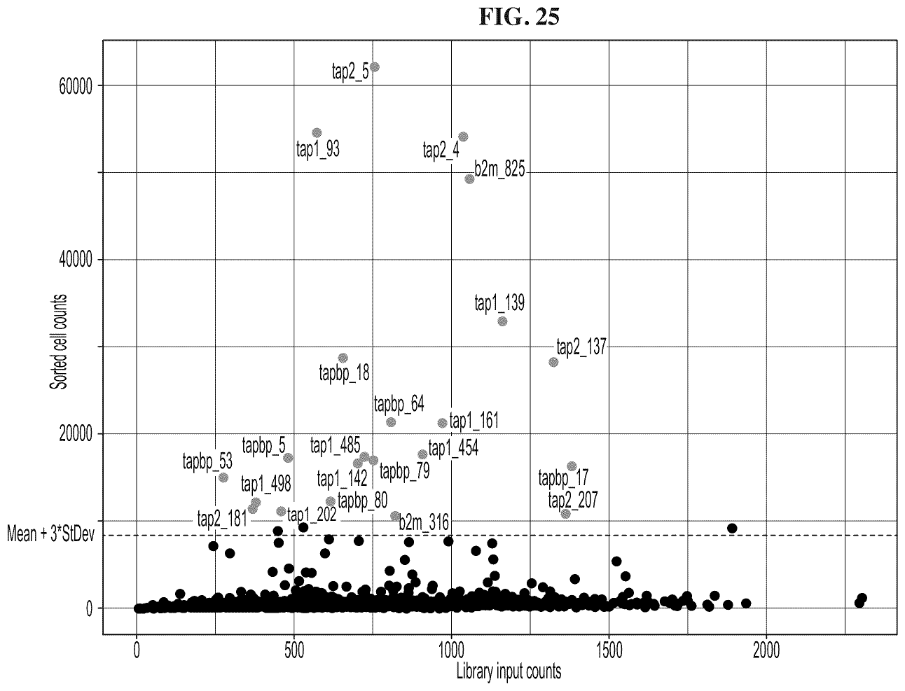

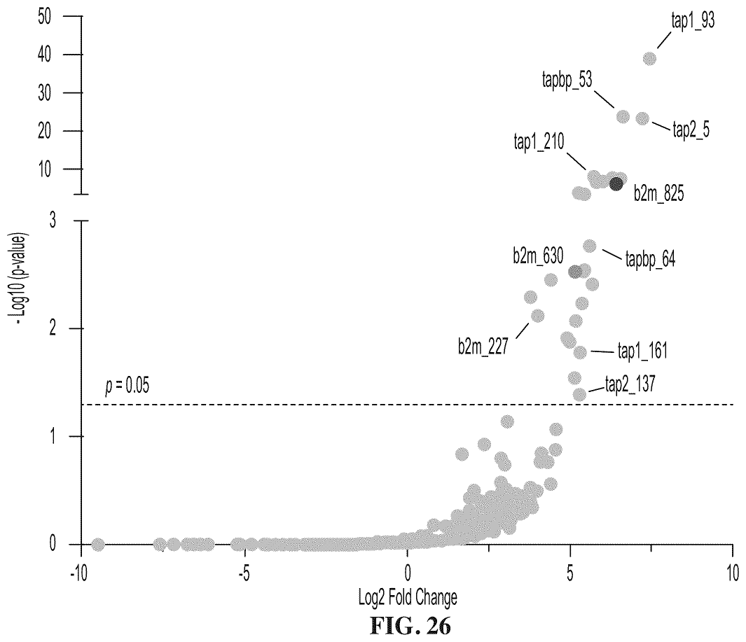

Next generation sequencing (NGS) was used to identify sgRNA sequences enriched in the sorted population. A plot of input library sgRNA sequence counts vs. sorted cell sgRNA sequence counts is provided in FIG. 25 and shows those sgRNA sequences enriched for in the sorted cells and that, therefore, resulted in reduced levels of B2M in the cells. A Model-based Analysis of Genome-wide CRISPR/Cas9 Knockout (MAGeCK) analysis to obtain p-values for the enriched sgRNA sequences is provided in FIG. 26. The enriched guides and their associated p-values are listed below in Table 20

TABLE 20
Guide RNA′s (sequences are provided in Table 1A) identified in a screen for
guide RNA′s that, when introduced into a cell with a CBE, were associated with a
reduction in B2M expression in the cell. The guide b2m_825 was a guide previously
known to be effective for use in combination with a CBE to knock-out B2M expression
in a cell.

| sgRNA | Target | LFC | p-value < 0.05 |
|---|---|---|---|
| tap1_93 | Ex. 1 Stop | 7.4401 | 7.03E−40 |
| tapbp_53 | Ex.3 SD | 6.6267 | 1.35E−24 |
| tap2_5 | Ex.1 Stop | 7.2276 | 4.36E−24 |
| tap1_210 | Ex.2 Stop | 5.7332 | 7.95E−09 |
| tapbp_18 | Ex.2 Stop | 6.3208 | 1.13E−08 |
| tap2_4 | Ex.1 Stop | 6.5715 | 2.22E−08 |
| tapbp_5 | Ex.1 SA | 6.0282 | 1.14E−07 |
| tap1_498 | Ex.11 Stop | 5.8727 | 1.41E−07 |
| tap2_181 | Ex.4 Stop | 5.8155 | 3.06E−07 |
| b2m_825 | Ex. 1 SA | 6.4103 | 7.14E−07 |
| tap1_140 | Ex. 1 Stop | 5.2768 | 0.0001201 |
| tap1_202 | Ex.2 SD | 5.4565 | 0.00029346 |
| tapbp_64 | Ex.3 Stop | 5.5922 | 0.0017232 |
| tap1_485 | Ex.10 SD | 5.4537 | 0.002869 |
| tap1_142 | Ex. 1 Stop | 5.4298 | 0.0029897 |
| b2m_630 | D to N, V to I | 5.1592 | 0.002997 |
| tap2_325 | G to E, V to M | 4.4261 | 0.0035467 |
| tap1_139 | Ex.1 Stop | 5.6938 | 0.0039119 |
| tap1_38 | ? | 3.7956 | 0.0051346 |
| tapbp_79 | Ex.3 Stop | 5.3643 | 0.0058662 |
| b2m_227 | ? | 4.0202 | 0.0075654 |
| tapbp_80 | Ex.3 Stop | 5.1797 | 0.008526 |
| b2m_596 | Ex.3 SD | 4.9235 | 0.012183 |
| tap1_444 | Ex.9 Stop | 5.0023 | 0.013265 |
| tap1_161 | Ex.1 Stop | 5.3186 | 0.016731 |
| tap1_454 | Ex.9 SA | 5.1507 | 0.028677 |
| tap2_137 | Ex.2 SA | 5.2829 | 0.040738 |

### Example 16: Direct and Specific Knock-Out of HLA Class-I Genes in T Cells to Improve Allogeneic Persistence in a Subject

Experiments were undertaken to identify guide RNAs useful in base editing to directly and specifically knock-out HLA class-I gene expression in T cells to improve allogeneic persistence of the cells in a subject. Experiments were also undertaken to determine the contribution of HLA-A, -B, and -C genetic knock-out in T cells to elimination of the T cells by alloreactive T cells or NK cells.

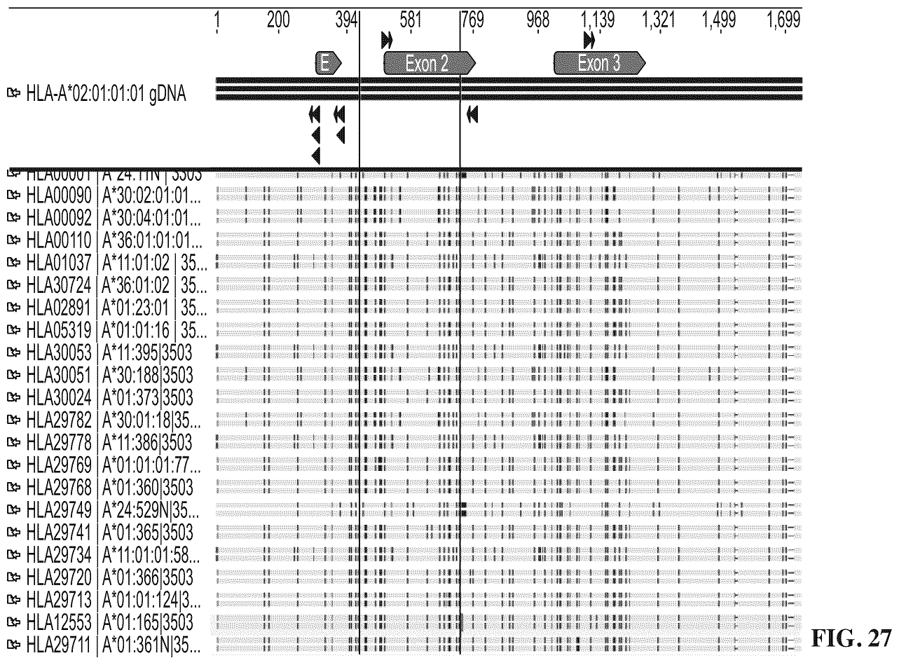

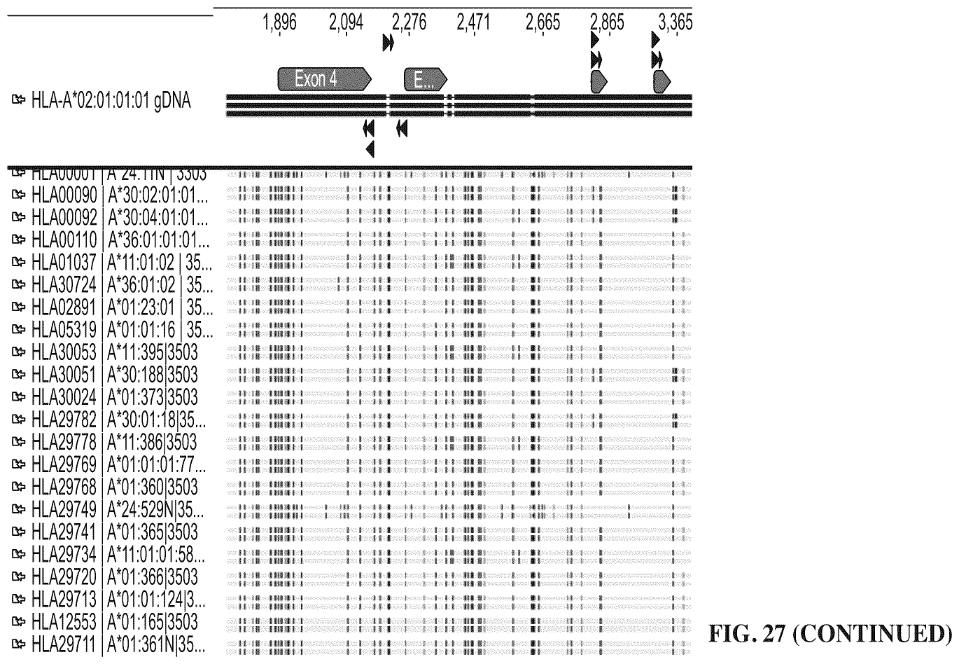

First, to design guide RNA sequences, HLA class-I gene sequences were aligned (see, e.g., FIG. 27) to identify conserved regions that could be targeted by guide RNAs. A criterion for the design of the guide RNAs was that they must hit conserved sites for HLA-A, —B, and/or -C. Conserved sites were observed in many cases among all HLA class-I alleles (up to 12 target alleles). Over 8000 HLA class-I genes were described at the time the alignments were prepared. Guides designed to target the HLA class-I genes are provided in Table 1A. Guides were designed that are suitable for use with ABE, CBE, and/or Cas12b. The guides targeted a start site, a splice acceptor site, a splice donor site, or exon conserved regions.

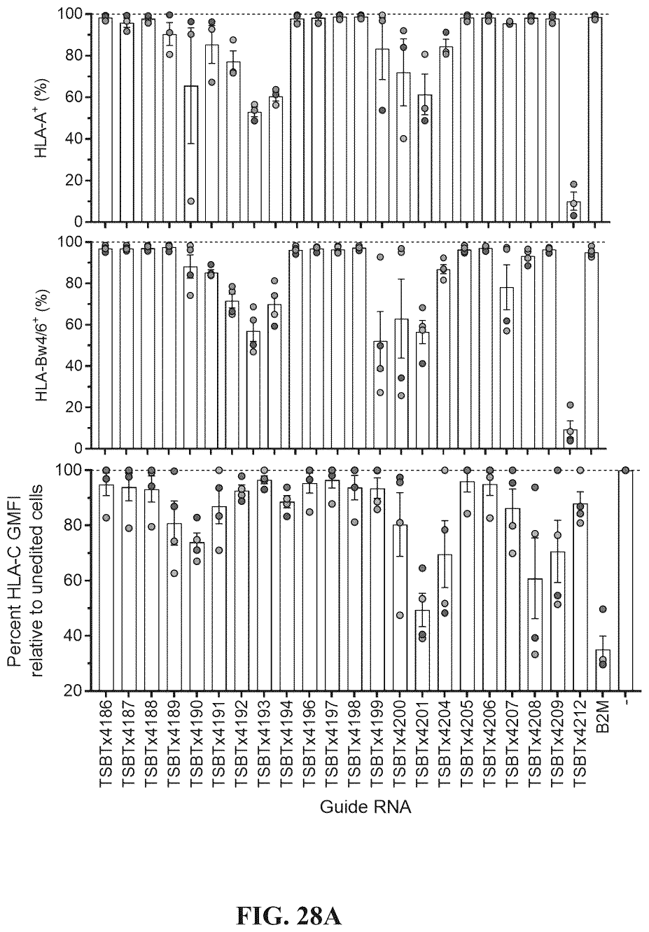

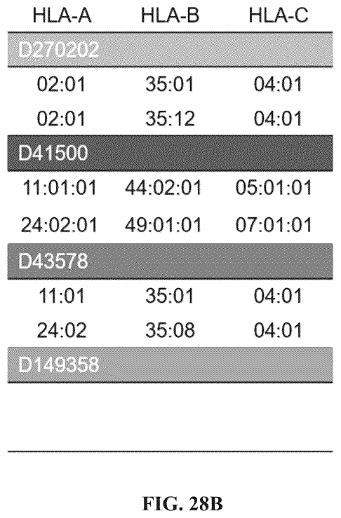

To screen the guide RNAs for effectiveness, the designed guides were used to introduce base edits into the genomes of T cells collected from four independent donors according to the method described herein. The resulting expression of HLA-A, HLA-B, and HLA-C was measured in the edited T cells (see FIGS. 28A and 28B). FIG. 28B provides the genotypes for three out of the four donors. β2M knock-out reduced HLA-A, -B, and -C expression relative to unedited cells. The g850 guide (TSBTx4190) specifically knocked out HLA-A*20, which contains a 1 nt mismatch from other HLA-A alleles. The g860 guide (TSBTx4200) specifically knocked out HLA-B expression. The TSBTx4193 (g853) and TSBTx4194 (g854) guides overlapped+1 nt targeting SD exon 4 (α3 domain).

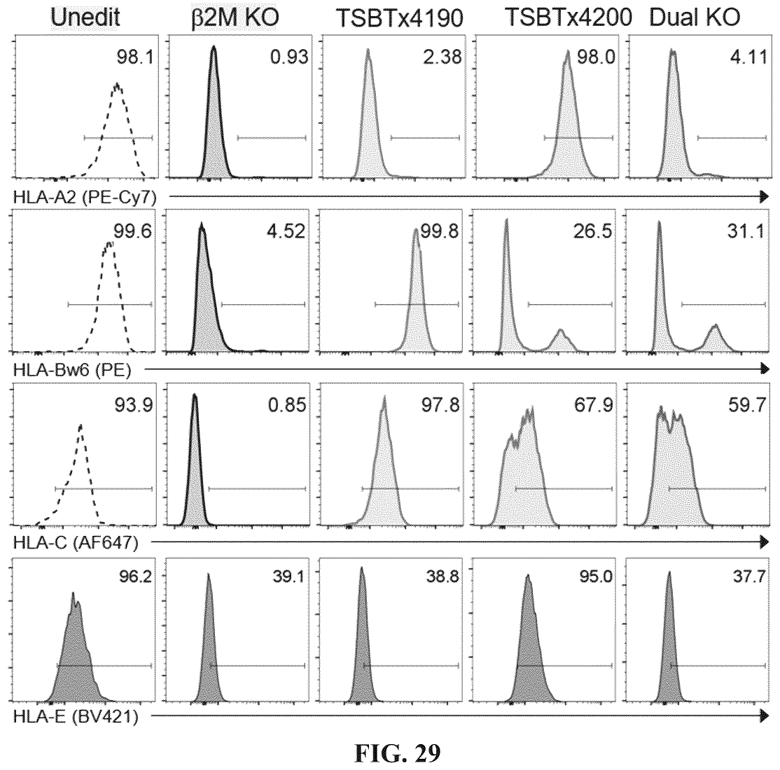

Synthetic guide RNAs TSBTx4190 (g850) and TSBTx4200 (g860) were designed to specifically disrupt HLA-A and HLA-B expression, respectively. Primary T cells from donor D270202 (see FIG. 28B) were base edited using TSBTx4190 and/or TSBTx4200 in combination with a ABE8.20m according to the methods described herein. Resulting HLA-A2, HLA-B, HLA-C, and pan HLA-E surface expression was measured using flow cytometry (see FIG. 29). As confirmed by flow cytometry, base editing using TSBTx4190 was associated with a specific reduction in HLA-A2 expression and base editing using TSBTx4200 was associated with a specific reduction in HLA-Bw6 expression. Base editing carried out using the guides TSBTx4190 and TSBTx4200 was not associated with a reduction in pan HLA-E expression.

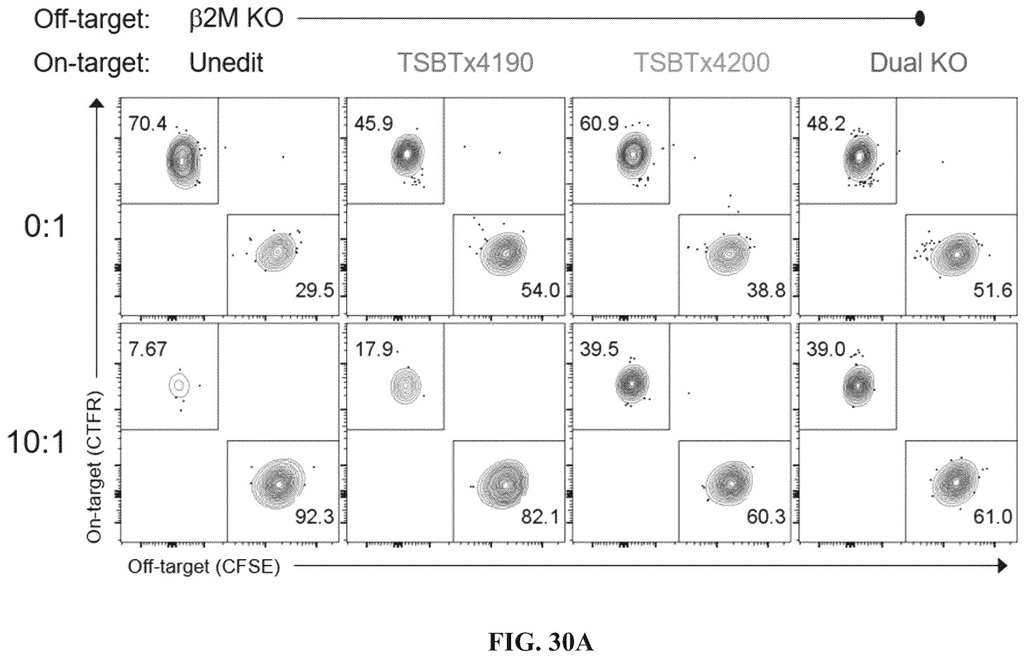

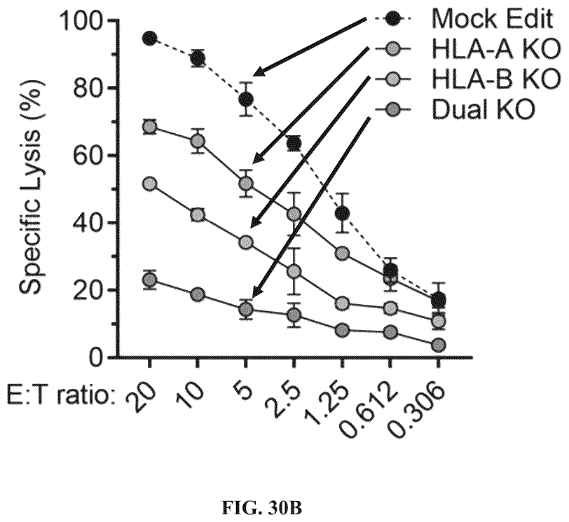

The targeted HLA class-I knock-outs mitigated T cell allorejection (see FIGS. 30A and 30B). An in vitro mixed leukocyte reaction was carried out according to methods described herein to evaluate specific killing of CFSE-labeled on-target cells including unedited (HLA+), HLA-A knock-out (TSBTx4190), and HLA-B knock-out (TSBTx4200) T cells relative to CTFR-labeled off-target β2M knock-out T cells by HA class-I mismatched effector T cells. Cell lysis was evaluated 48 hours post-coculture. As can be seen from FIGS. 30A and 30B, base editing using ABE8.20m in combination with TSBTx4190 and/or TSBTx4200 resulted in reduced killing by the mismatched effector T cells.

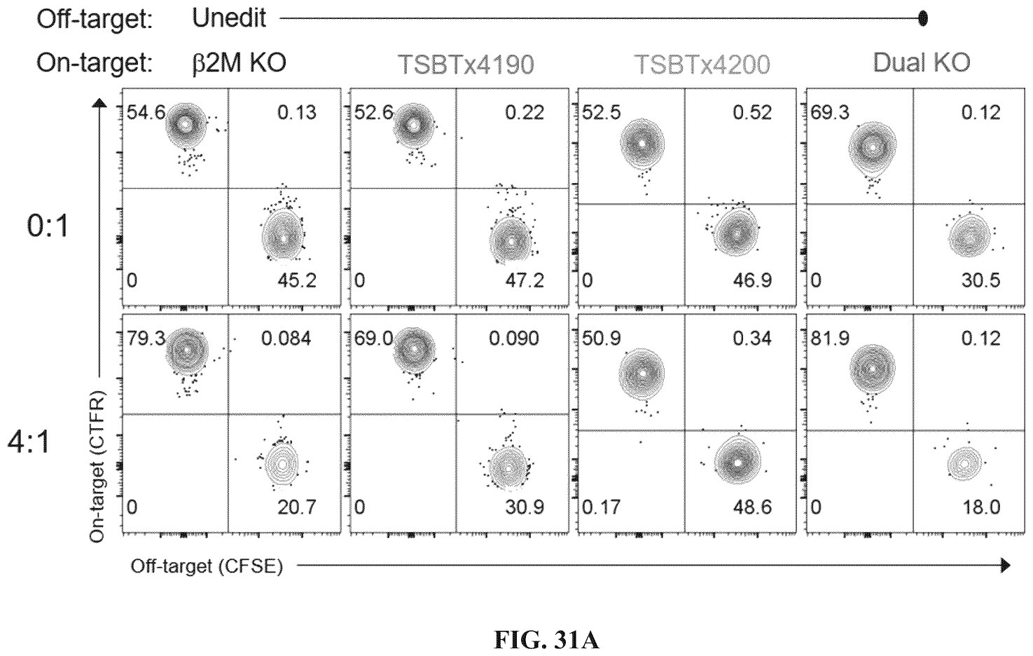

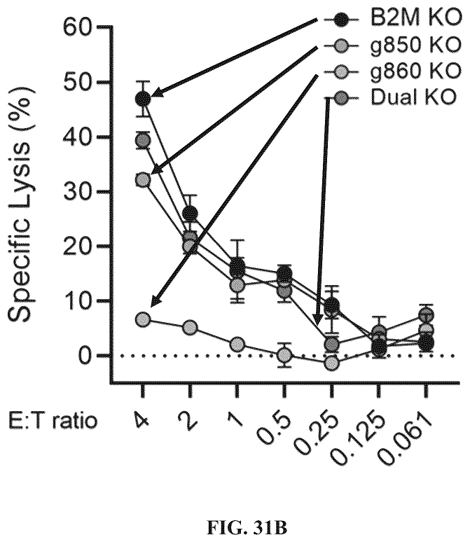

Targeted knock-out of HLA-B using g860 (TSBTx4200) in combination with ABE8.20m was associated with protection against NK cell lysis (see FIGS. 31A and 31B). An in vitro mixed leukocyte reaction was carried out according to methods described herein to evaluate specific killing of CTFR-labeled on-target cells including β2M knock-out, HLA-A knock-out (TSBTx4190), and HLA-B knock-out (TSBTx4200) T cells relative to CFSE-labeled off-target unedited (HLA+) T cells. Cell lysis was measured 48 hours post-culture. The plots of FIGS. 31A and 31B demonstrate that HLA-B was dispensable for protection against NK cell lysis.

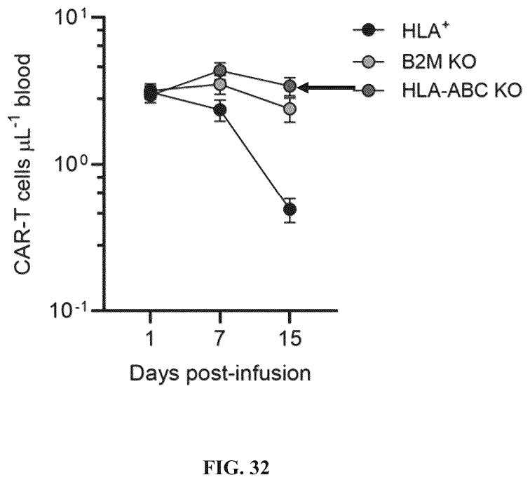

An experiment was undertaken to determine whether specific HLA-ABC knock-out prevented T cell-mediated allorejection in vivo. BLT mice were co-infused with an equal mixture of unedited (HLA+), β2M knock-out, and HLA-ABC knock-out T cells. The TRAC and CIITA genes of all T cells were also knocked out using base editing. Base editing of all genes was carried out using ABE8.20m according to the methods described herein and using guides listed in Table 1A. HLA-ABC knock-out T cells were additionally multiplex edited with the following guide RNAs: TSBTx4190, which targets HLA-A, TSBTx4201, which targets HLA-B, and TSBTx4208, which targets HLA-C (see Table 1A). Table 21 provides an overview of how the cells were modified. The graph of FIG. 32 (see also Table 21) shows peripheral blood concentration of each T cell population over time post-infusion. HLA+ T cells were susceptible to allorejection but the β2M knock-out and HLA-ABC knock-out cells were resistant to allorejection.

TABLE 21
Modifications made to the cells used to prepare FIG. 32. RQR8
was used as a surface receptor tag to identify T cells.

| | | Edits | | | |
|---|---|---|---|---|---|
| | T cell Tag | TCR | HLA-II | HLA-I | Target |
| equal | RQR8 | CD3e | CIITA | — | ON |
| mix | RQR8 | CD3e | CIITA | b2M | OFF |
| | GFP | CD3e | CIITA | ABC | OFF |

These results demonstrate that base editing could be leveraged to knock-out HLA-A, HLA-B, and HLA-C directly across multiple HLA mismatched donors. Knockout of these genes had a differential impact on T cell and NK cell mediated allorejection.

### Example 22: Base-Edited Allogeneic T Cells Expressing HLA-C+ but Deficient in Expression of HLA-A and HLA-B were Resistant to Rejection

Experiments were undertaken to prepare modified allogeneic immune cells resistant to elimination by a host's natural killer cells and/or T cells.

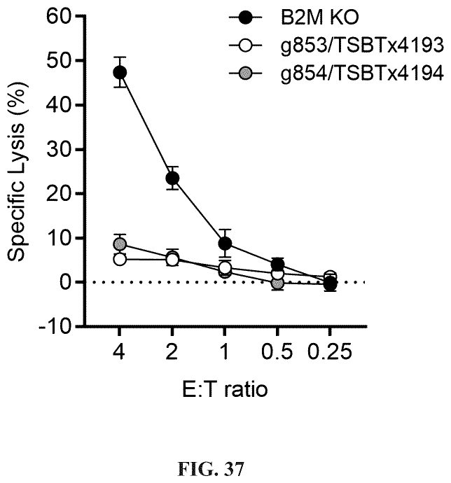

First, in vitro experiments were undertaken to demonstrate that cell populations deficient in expression of HLA-A, HLA-B, and/or HLA-C were resistant to killing by natural killer cells or T cells in mixed leukocyte reactions. T cell populations were edited using the base editor ABE8.20m in combination with a guide RNA targeting beta-2-microglobulin (see sequences listed in Tables 1A and SEQ ID NOs: 2448, 2450, 2454, and 2622-2625) or with one of the pan HLA class-I guide RNAs TSBTx4193 and TSBTx4194 (see sequences listed in Tables TA and 1C). The base edited T cell populations were then evaluated using an NK cell mixed leukocyte reaction. The Effector (“E”) cells (natural killer (NK) cells) and Target (“T”) cells (base-edited cells) were cultured for 48 hours and specific lysis was then assessed using flow cytometry. T cells edited with the pan HLA class-I guide RNAs were protected from NK cell lysis in vitro, while beta-2-microglobulin knock-out cells were susceptible to lysis, as shown in FIG. 37.

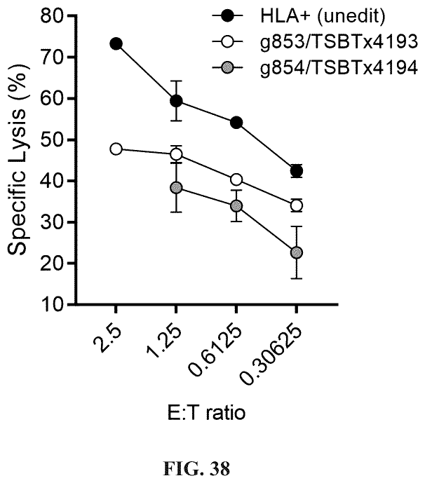

The base edited T cell populations were also evaluated using a T cell mixed leukocyte reaction. Primary human T cells were co-cultured with either unedited HLA class-I mismatched T cells or the edited T cell populations. The effector cells (mismatched T cells) and target cells (base-edited cells) were cultured for 48 hours and specific lysis was then assessed using flow cytometry. The T cell populations edited with the pan HLA class-I guide RNAs showed increased protection from T cell lysis in vitro relative to unedited HLA+ mismatched T cells, as shown in FIG. 38.

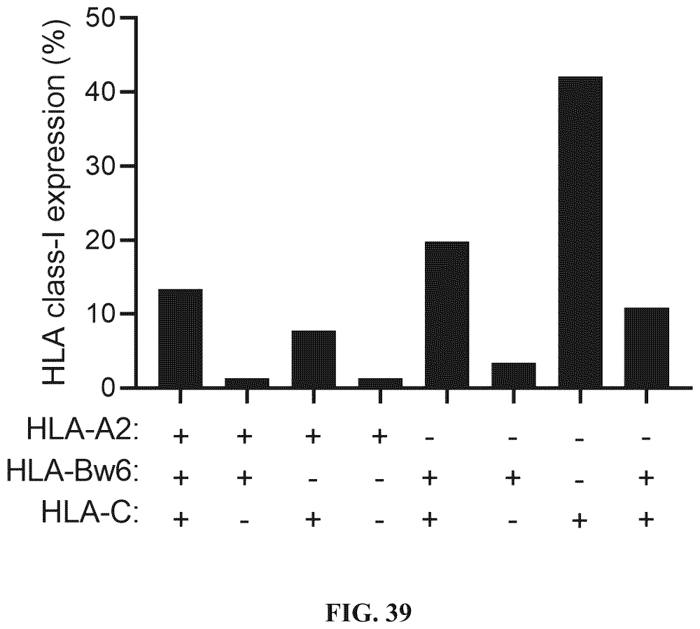

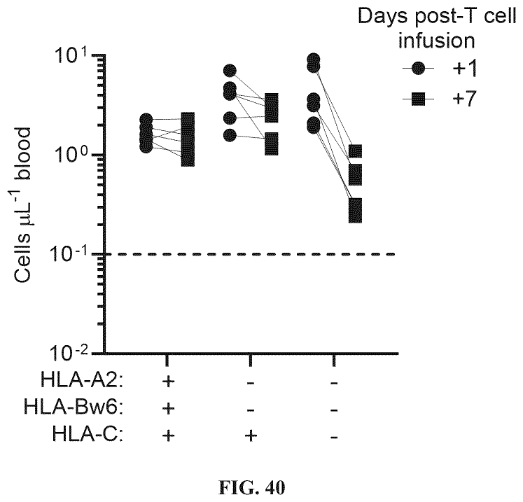

Having determined that T cell populations base edited using the base editor ABE8.20m in combination with the guide RNA TSBTx4193 were resistant to lysis in vitro by natural killer (NK) or mismatched T cells, an experiment was undertaken to determine the immunophenotypes of cells in the edited T cell populations. Surface-expression of HLA-A, HLA-B, and HLA-C on the cells was measured by staining the cells using antibodies against HLA-A2, HLA-Bw6, and HLA-C, respectively, followed by flow cytometry. The immunophenotype HLA-A2- (i.e., no surface-expression of HLA-A2), HLA-Bw6- (i.e., no surface expression of HLA-Bw6), and HLA-C+(i.e., positive for surface expression of HLA-C) was the dominant immunophenotype in the base edited T cell population, as shown in FIG. 39.

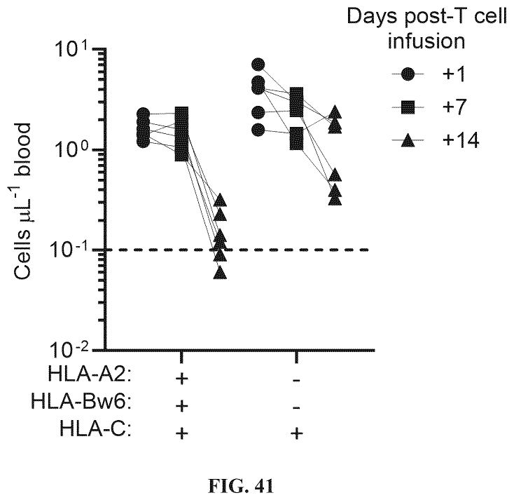

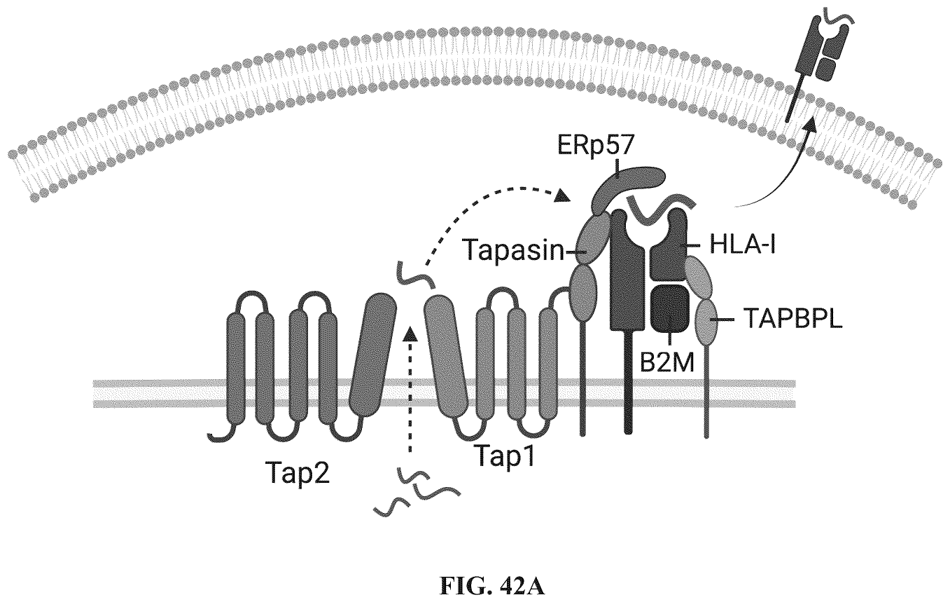

Experiments were next undertaken to evaluate the persistence of the base-edited allogeneic T cells in vivo. IL-15 primed BLT mice were infused with the base-edited allogeneic T cell populations and the number of allogeneic T cells having the immunophenotype HLA-ABC+(i.e., HLA-A+, HLA-B+, and HLA-C+), HLA-C+(i.e., HLA-A-, HLA-B-, and HLA-C+), or HLA-ABC− (i.e., HLA-A-, HLA-B-, and HLA-C-) was counted using flow cytometry at days 1, 7, and 14 post-infusion (see FIGS. 40 and 41). As determined through measurements taken at day 7 post-infusion (FIG. 40), HLA-ABC negative or HLA class-I deficient allogeneic T cells were susceptible to NK cell-mediated rejection in vivo, while allogeneic T cells expressing HLA-ABC+ or expressing only HLA-C and not HLA-A or HLA-B were relatively resistant to NK cell-mediated rejection in vivo. As determined through measurements taken at day 14 post-infusion (FIG. 41), HLA-ABC+ allogeneic T cells were less resistant to T cell-mediated rejection in vivo than T cells expressing HLA-C and not HLA-A or HLA-B.

Therefore, immune cells base edited to be deficient in expression of HLA-A and HLA-B while maintaining expression of HLA-C showed improved resistance to both NK- and T cell-mediated rejection in vivo relative to both unedited cells and cells deficient in expression of all of HLA-A, HLA-B, and HLA-C.

### Example 23: Base Editing Using Guides Targeting Beta-2-Microglobulin (B2M) or a Single Component of the Peptide Loading Complex Resulted in Downregulated B2M Surface-Expression but not of HLA Class-I Polypeptides

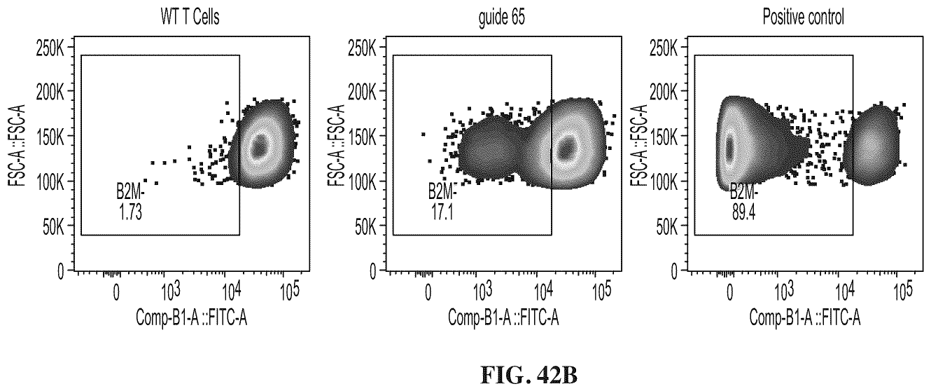

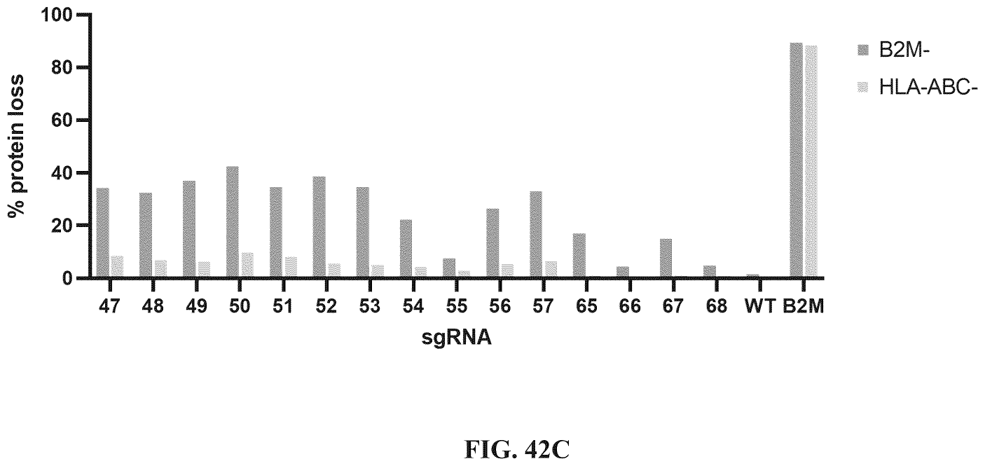

Experiments were undertaken to evaluate the impact of base editing using guide RNAs targeting a component of the peptide loading complex (i.e., TAP2, TAP1, TAPBP, FIG. 42A), or B2M on surface-expression of B2M and HLA-A, -B, and -C (FIGS. 42B and 42C). Guides 47-68 were evaluated in combination with a base editor to edit T cells, where guide 47 corresponded to Tap2_5, which targets TAP2; guide 48 corresponded to Tap1_93, which targets TAP1; guide 49 corresponded to Tap2_4, which targets TAP2; guide 50 corresponded to Tap1_139, which targets TAP1; guide 51 corresponded to Tapbp_18, which targets TAPBP; guide 52 corresponded to Tap2_137, which targets TAP2; guide 53 corresponded to Tapbp_64, which targets TAPBP; guide 54 corresponded to Tap1_161, which targets TAP1; guide 55 corresponded to Tap1_454, which targets TAP1; guide 56 corresponded to Tap1_485, which targets TAP1; guide 57 corresponded to Tapbp_5, which targets TAPBP; guide 58 corresponded to b2m_629, which targets B2M; guide 59 corresponded to b2m_630, which targets B2M; guide 60 corresponded to b2m_315, which targets B2M; and guide 61 corresponded to b2m_316, which targets B2M (see Table 1A for guide sequences). A B2M guide RNA known to be effective for use in knocking out B2M expression was used as a positive control and corresponded to TSBTx845 (see Table 1A).

It was found that base editing using some guides targeting a polynucleotide encoding a single component of the peptide loading complex or B2M resulted in reduced surface-expression of B2M but had little-to-no influence on surface-expression of HLA-A, -B, and -C.

### Example 24: Multiplex Editing Tuned HLA Class-I Polypeptide Surface-Expression

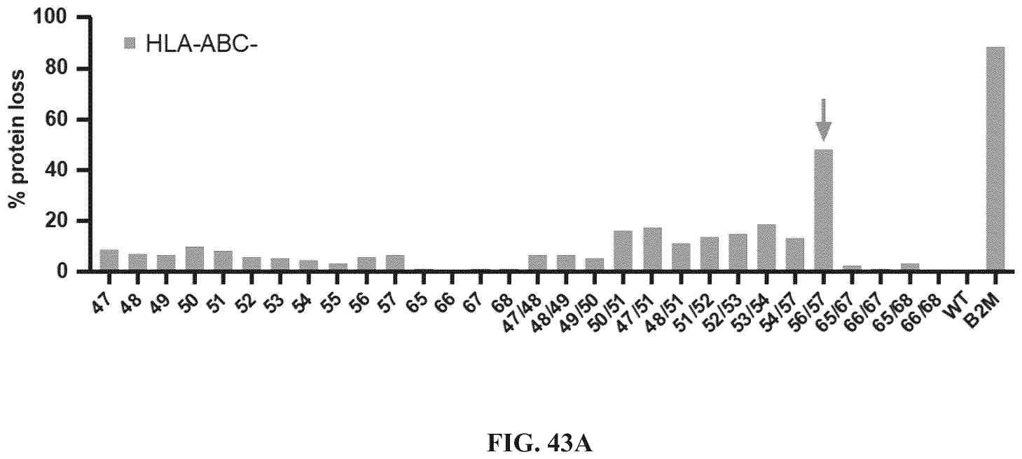

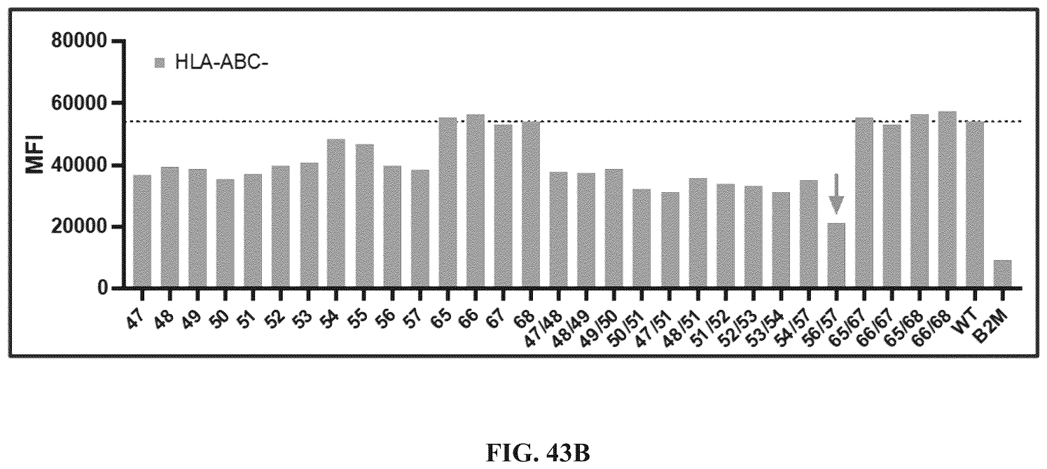

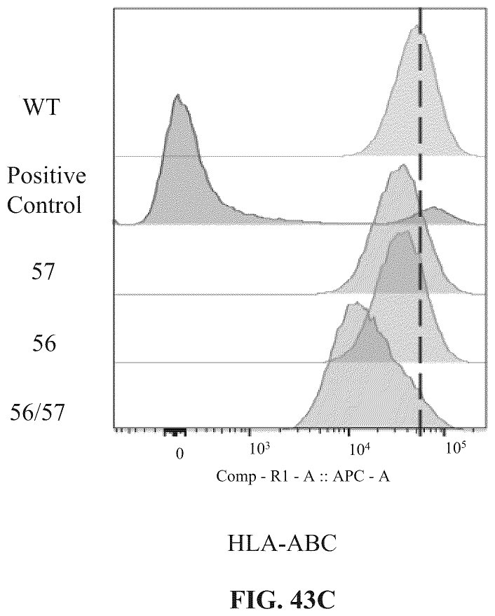

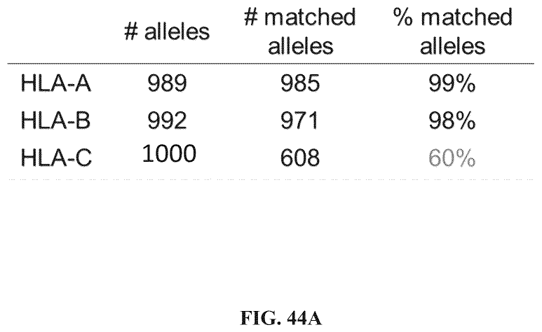

Having determined that editing of a single polynucleotide was sometimes not effective in downregulating surface-expression of HLA Class-I polypeptides (i.e., HLA-A, -B, and -C) in base-edited T cells, experiments were next undertaken to determine if multiplex editing would result in reduced HLA Class-I polypeptide expression. Combinations of 47-68 (see FIGS. 43A-43C) were evaluated in combination with a base editor to edit T cells, where guide 47 corresponded to Tap2_5, which targets TAP2; guide 48 corresponded to Tap1_93, which targets TAP1; guide 49 corresponded to Tap2_4, which targets TAP2; guide 50 corresponded to Tap1_139, which targets TAP1; guide 51 corresponded to Tapbp_18, which targets TAPBP; guide 52 corresponded to Tap2_137, which targets TAP2; guide 53 corresponded to Tapbp_64, which targets TAPBP; guide 54 corresponded to Tap1_161, which targets TAP1; guide 55 corresponded to Tap1_454, which targets TAP1; guide 56 corresponded to Tap1_485, which targets TAP1; guide 57 corresponded to Tapbp_5, which targets TAPBP; guide 58 corresponded to b2m_629, which targets B2M; guide 59 corresponded to b2m_630, which targets B2M; guide 60 corresponded to b2m_315, which targets B2M; and guide 61 corresponded to b2m_316, which targets B2M (see Table 1A for guide sequences). A B2M guide RNA known to be effective for use in knocking out B2M expression was used as a positive control and corresponded to TSBTx845 (see Table 1A).

It was found that multiplex editing could be used to effectively tune surface-expression of HLA class-I polypeptides (i.e., HLA-A, -B, and -C). In particular, it was found that the combination of guide 56 targeting TAP1 and guide 57 targeting tapasin effected a reduction in surface-expression of the HLA class-I polypeptides (FIGS. 43A-43C). Therefore, it was found that base editing of polynucleotides encoding multiple components of the peptide loading complex had a tuning effect by reducing but not eliminating surface-expression of the HLA class-I polypeptides.

### Example 25: Sequence Alignments Indicated that Base Editing could be Used to Preferentially Knock-Out HLA-A and HLA-B Alleles while not Knocking-Out HLA-C Expression

Experiments were undertaken to evaluate, in silico, the capacity for guide RNAs TSBTx4193 and TSBTx4194 to target alleles encoding HLA class-I polypeptides. As shown in FIG. 44A, it was determined that over 99% of 989 HLA-A alleles evaluated and 98% of 992 HLA-B alleles evaluated could be targeted using the guides, while only 60% of 1000 HLA-C alleles evaluated could be targeted. Therefore, it was determined that cells expressing HLA-C and with HLA-A and HLA-B expression knocked out could be prepared by base-editing using the guide RNA TSBTx4193 and/or TSBTx4194 followed by cell sorting (e.g., using flow cytometry) to select for those cells surface-expressing HLA-C and not surface-expressing HLA-A or HLA-B.

It was determined through use of multiple sequence alignments (see FIG. 44B) that some HLA-C alleles (see, e.g., HLA-C*02:02:02:01) could be effectively targeted using TSBTx4193 or TSBTx4194 while other HLA-C alleles (e.g., HLA-C*17:01:01:02) could not on account of mutations, such as a mutation within the PAM sequence recognized by a base editor used in combination with the guides. Thus, careful selection of a donor could be used to obtain cells that, when base edited with TSBTx4193 or TSBTx4194, do not express HLA-A or HLA-B, but do express HLA-C.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for producing a persistent allogeneic modified immune cell, the method comprising contacting a cell with a base editor comprising a polynucleotide programmable DNA binding polypeptide (napDNAbp), a deaminase, and one or more guide RNAs (gRNAs) that target the base editor to effect an alteration in a nucleic acid molecule, wherein the nucleic acid molecule encodes a polypeptide and/or comprises a regulatory element associated with expression thereof, and wherein the polypeptide is selected from the group consisting of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP), thereby producing the persistent allogeneic modified immune cell.

2. The method of claim 1, wherein the method further comprises contacting the cell with one or more guide RNAs that target the base editor to effect an alteration in a nucleic acid molecule, wherein the nucleic acid molecule encodes a polypeptide and/or comprises a regulatory element associated with expression thereof, and wherein the polypeptide is selected from the group consisting of beta-2 microglobulin, CD48, CD58, Protein Disulfide Isomerase Family A Member 3 (PDIA3/ERp57), and T Cell Receptor Alpha Constant polypeptides.

3. The method of claim 1, wherein the method comprises effecting a nucleobase alteration that reduces expression on the cell of a polypeptide selected from the group consisting of HLA-A, HLA-B, and HLA-C.

4. The method of claim 1, wherein the one or more gRNAs comprise a nucleotide sequence with at least about 85% sequence identity to GCACUCACCCGCCCAGGUCU (SEQ ID NO: 817; TSBTx4190), GACCCGCAUCUCGGCGUCUG (SEQ ID NO: 827; TSBTx4200), CCUUACCCCAUCUCAGGGUG (SEQ ID NO: 820; TSBTx4193), and/or CUUACCCCAUCUCAGGGUGA (SEQ ID NO: 821; TSBTx4194).

5. The method of claim 1, further comprising overexpressing in the cell an inhibitory receptor, or fragment thereof, selected from the group consisting of Human Leukocyte Antigen-E (HLA-E), Human Leukocyte Antigen-G (HLA-G), Programmed Death Ligand 1 (PD-L1), and Cluster of Differentiation 47 (CD47).

6. A method for producing a persistent allogeneic modified immune cell, the method comprising:
(a) contacting a cell with a base editor comprising a polynucleotide programmable DNA binding polypeptide (napDNAbp), a deaminase, and one or more guide RNAs (gRNAs) that target a nucleic acid molecule, wherein the nucleic acid molecule encodes a polypeptide or comprises a regulatory element associated with expression of the polypeptide, and wherein the polypeptide is selected from the group consisting of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP); and
(b) overexpressing in the cell an inhibitory receptor, or fragment thereof, selected from the group consisting of Human Leukocyte Antigen-E (HLA-E), Human Leukocyte Antigen-G (HLA-G), Programmed Death Ligand 1 (PD-L1), and Cluster of Differentiation 47 (CD47).

7. An allogeneic modified immune cell produced according to the method of claim 1.

8. An allogeneic modified immune cell comprising a nucleobase alteration that reduces or eliminates expression of a polypeptide selected from the group consisting of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP).

9. A pharmaceutical composition comprising an effective amount an allogeneic modified immune cell of claim 8.

10. A composition comprising a guide RNA (gRNA) and a polynucleotide encoding a base editor comprising a polynucleotide programmable DNA binding polypeptide (napDNAbp) domain and a deaminase domain, wherein the gRNA comprises a nucleic acid sequence that is complementary to a polynucleotide, wherein the polynucleotide encodes a polypeptide or comprises a regulatory element associated with expression of the polypeptide, wherein the polypeptide is selected from the group consisting of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP).

11. The composition of claim 10, wherein the guide RNA comprises a nucleotide sequence selected from the group consisting of GCACUCACCCGCCCAGGUCU (SEQ ID NO: 817; TSBTx4190), GACCCGCAUCUCGGCGUCUG (SEQ ID NO: 827; TSBTx4200), CCUUACCCCAUCUCAGGGUG (SEQ ID NO: 820; TSBTx4193), and CUUACCCCAUCUCAGGGUGA (SEQ ID NO: 821; TSBTx4194).

12. A kit comprising an allogeneic modified immune cell of claim 8.

13. A method of treating cancer in a subject, the method comprising administering to the subject an effective amount of an allogeneic modified immune cell of claim 8.

14. A fusion polypeptide comprising a loading peptide, at least a fragment of an HLA-G polypeptide, and at least a fragment of a β2M polypeptide.

15. The fusion polypeptide of claim 14, wherein the recombinant polypeptide comprises from N-terminus to C-terminus:
a) a loading peptide, at least a fragment of an HLA-G polypeptide, and at least a fragment of a β2M polypeptide;
b) at least a fragment of a β2M polypeptide, a loading peptide, and at least a fragment of an HLA-G polypeptide;
c) a loading peptide, at least a fragment of a β2M polypeptide, and at least a fragment of an HLA-G polypeptide; or
d) fragment of an HLA-G polypeptide, a loading peptide, and at least at least a fragment of a β2M polypeptide.

16. A fusion polypeptide comprising a loading peptide, at least a fragment of an HLA-E polypeptide, and at least a fragment of a β2M polypeptide.

17. A fusion polypeptide comprising a loading peptide, and at least a fragment of an HLA-E polypeptide.

18. A fusion polypeptide comprising an amino acid sequence with at least 85% sequence identity to a sequence selected from the group consisting of:
HLA-G5 + IL-2 signal peptide
(SEQ ID NO: 1013)
MYRMQLLSCIALSLALVTNSGSHSMRYFSAAVSRPGRGEPRFIAMGYVDD
TQFVRFDSDSACPRMEPRAPWVEQEGPEYWEEETRNTKAHAQTDRMNLQT
LRGYYNQSEASSHTLQWMIGCDLGSDGRLLRGYEQYAYDGKDYLALNEDL
RSWTAADTAAQISKRKCEAANVAEQRRAYLEGTCVEWLHRYLENGKEMLQ
RADPPKTHVTHHPVFDYEATLRCWALGFYPAEIILTWQRDGEDQTQDVEL
VETRPAGDGTFQKWAAVVVPSGEEQRYTCHVQHEGLPEPLMLRWSKEGDG
GIMSVRESRSLSEDL;
HLA-G5 Single chain trimer + IL-2 signal peptide
(SEQ ID NO: 1014)
MYRMQLLSCIALSLALVTNSIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDMGGGGSGGGGSGGGGSRIIPRHLQLGGGGSGG
GGSGGGGSGGGGSGSHSMRYFSAAVSRPGRGEPRFIAMGYVDDTQFVRFD
SDSACPRMEPRAPWVEQEGPEYWEEETRNTKAHAQTDRMNLQTLRGYYNQ
SEASSHTLQWMIGCDLGSDGRLLRGYEQYAYDGKDYLALNEDLRSWTAAD
TAAQISKRKCEAANVAEQRRAYLEGTCVEWLHRYLENGKEMLQRADPPKT
HVTHHPVFDYEATLRCWALGFYPAEIILTWQRDGEDQTQDVELVETRPAG
DGTFQKWAAVVVPSGEEQRYTCHVQHEGLPEPLMLRWSKEGDGGIMSVRE
SRSLSEDL;
HLA-E(ΔTM) Single chain trimer + HLA-G5 intron
tail
(SEQ ID NO: 1015)
MSRSVALAVLALLSLSGLEAVMAPRTLFLGGGGSGGGGSGGGGSIQRTPK
IQVYSRHPAENGKSNFLNCYVSGFHPSDIEVDLLKNGERIEKVEHSDLSF
SKDWSFYLLYYTEFTPTEKDEYACRVNHVTLSQPKIVKWDRDMGGGGSGG
GGSGGGGSGGGGSGSHSLKYFHTSVSRPGRGEPRFISVGYVDDTQFVRFD
NDAASPRMVPRAPWMEQEGSEYWDRETRSARDTAQIFRVNLRTLRGYYNQ
SEAGSHTLQWMHGCELGPDGRFLRGYEQFAYDGKDYLTLNEDLRSWTAVD
TAAQISEQKSNDASEAEHQRAYLEDTCVEWLHKYLEKGKETLLHLEPPKT
HVTHHPISDHEATLRCWALGFYPAEITLTWQQDGEGHTQDTELVETRPAG
DGTFQKWAAVVVPSGEEQRYTCHVQHEGLPEPVTLRWSKEGDGGIMSVRE
SRSLSEDL;
HLA-E(ΔTM) β2M (C-term) Single chain trimer
(SEQ ID NO: 1016)
MSRSVALAVLALLSLSGLEAVMAPRTLFLGGSGGGASGGGSHSLKYFHTS
VSRPGRGEPRFISVGYVDDTQFVRFDNDAASPRMVPRAPWMEQEGSEYWD
RETRSARDTAQIFRVNLRTLRGYYNQSEAGSHTLQWMHGCELGPDGRFLR
GYEQFAYDGKDYLTLNEDLRSWTAVDTAAQISEQKSNDASEAEHQRAYLE
DTCVEWLHKYLEKGKETLLHLEPPKTHVTHHPISDHEATLRCWALGFYPA
EITLTWQQDGEGHTQDTELVETRPAGDGTFQKWAAVVVPSGEEQRYTCHV
QHEGLPEPVTLRWGGGGSGGGGSGGGGSIQRTPKIQVYSRHPAENGKSNF
LNCYVSGFHPSDIEVDLLKNGERIEKVEHSDLSFSKDWSFYLLYYTEFTP
TEKDEYACRVNHVTLSQPKIVKWDRDM;
HLA-E(ΔTM) Single chain dimer + HLA-G5 intron
tail
(SEQ ID NO: 1017)
MSRSVALAVLALLSLSGLEAVMAPRTLFLGGSGGGASGGGSHSLKYFHTS
VSRPGRGEPRFISVGYVDDTQFVRFDNDAASPRMVPRAPWMEQEGSEYWD
RETRSARDTAQIFRVNLRTLRGYYNQSEAGSHTLQWMHGCELGPDGRFLR
GYEQFAYDGKDYLTLNEDLRSWTAVDTAAQISEQKSNDASEAEHQRAYLE
DTCVEWLHKYLEKGKETLLHLEPPKTHVTHHPISDHEATLRCWALGFYPA
EITLTWQQDGEGHTQDTELVETRPAGDGTFQKWAAVVVPSGEEQRYTCHV
QHEGLPEPVTLRWSKEGDGGIMSVRESRSLSEDL;
and
HLA-E(ΔTM) Single chain dimer
(SEQ ID NO: 1018)
MSRSVALAVLALLSLSGLEAVMAPRTLFLGGSGGGASGGGSHSLKYFHTS
VSRPGRGEPRFISVGYVDDTQFVRFDNDAASPRMVPRAPWMEQEGSEYWD
RETRSARDTAQIFRVNLRTLRGYYNQSEAGSHTLQWMHGCELGPDGRFLR
GYEQFAYDGKDYLTLNEDLRSWTAVDTAAQISEQKSNDASEAEHQRAYLE
DTCVEWLHKYLEKGKETLLHLEPPKTHVTHHPISDHEATLRCWALGFYPA
EITLTWQQDGEGHTQDTELVETRPAGDGTFQKWAAVVVPSGEEQRYTCHV
QHEGLPEPVTLRW.

19. A membrane-bound fusion polypeptide, wherein the fusion polypeptide comprises a β2M domain, and an HLA-E domain and/or a transmembrane domain.

20. A fusion polypeptide comprising an amino acid sequence having at least 85% sequence identity to the following sequence:
(SEQ ID NO: 1019)
MSRSVALAVLALLSLSGLEAVMAPRTLFLGGGGSGGGGSGGGGSIQRTPK
IQVYSRHPAENGKSNFLNCYVSGFHPSDIEVDLLKNGERIEKVEHSDLSF
SKDWSFYLLYYTEFTPTEKDEYACRVNHVTLSQPKIVKWDRDMGGGGSGG
GGSGGGGSGGGGSGSHSLKYFHTSVSRPGRGEPRFISVGYVDDTQFVRFD
NDAASPRMVPRAPWMEQEGSEYWDRETRSARDTAQIFRVNLRTLRGYYNQ
SEAGSHTLQWMHGCELGPDGRFLRGYEQFAYDGKDYLTLNEDLRSWTAVD
TAAQISEQKSNDASEAEHQRAYLEDTCVEWLHKYLEKGKETLLHLEPPKT
HVTHHPISDHEATLRCWALGFYPAEITLTWQQDGEGHTQDTELVETRPAG
DGTFQKWAAVVVPSGEEQRYTCHVQHEGLPEPVTLRWKPASQPTIPIMAL
IVLGGVAGLLLFIGLGIFFCVRC.

21. A mammalian expression vector comprising a polynucleotide sequence encoding the fusion polypeptide of claim 14.

22. An allogeneic modified immune cell comprising the vector of claim 21.

23. A method for producing a persistent allogeneic modified immune cell, the method comprising contacting a cell with a polynucleotide programmable DNA binding polypeptide (napDNAbp) and one or more guide RNAs (gRNAs) that target the napDNAbp to cleave a target nucleic acid molecule and introduce an alteration in the target nucleic acid molecule, wherein the target nucleic acid molecule encodes a polypeptide and/or comprises a regulatory element associated with expression thereof, and wherein the polypeptide is selected from the group consisting of HLA-A, HLA-B, HLA-C, Transporter Associated with Antigen Processing I (TAP1), Transporter Associated with Antigen Processing II (TAP2), Tapasin/TAP Binding Protein (TAPBP), TAP-Binding Protein-Like (TAPBPL), NLR family CARD domain containing 5 (NLRC5)/MHC class I transactivator (CITA), cluster of differentiation 155 (CD155), MHC class I polypeptide-related sequence A (MICA), MHC class I polypeptide-related sequence B (MICB) polypeptide, nectin cell adhesion molecule 2 (Nectin-2), and UL16 binding protein 1-6 (ULBP), thereby producing the persistent allogeneic modified immune cell.

24. A method for producing a persistent allogeneic modified immune cell, the method comprising contacting a cell with a base editor comprising a polynucleotide programmable DNA binding polypeptide (napDNAbp), a deaminase, and guide RNAs (gRNAs) that target the base editor to effect an alteration in one or more nucleic acid molecules, wherein the one or more nucleic acid molecules encode the following polypeptides and/or comprise regulatory elements associated with expression thereof: CD5, B2M, CD3 gamma, CD3 epsilon, CIITA, and PD-1 (PD1), thereby producing the persistent allogeneic modified immune cell; or contacting a cell with a base editor comprising a polynucleotide programmable DNA binding polypeptide (napDNAbp), a deaminase, and guide RNAs (gRNAs) that target the base editor to effect an alteration in one or more nucleic acid molecules, wherein the one or more nucleic acid molecules encode the following polypeptides and/or comprise regulatory elements associated with expression thereof: HLA-A, HLA-B, and CIITA, thereby producing the persistent allogeneic modified immune cell, wherein the persistent allogeneic modified immune cell surface-expresses HLA-C.

25. An allogeneic modified immune cell produced by the method of claim 24.

26. A method of treating cancer in a subject, the method comprising administering to the subject an effective amount of the allogeneic modified immune cell of claim 25.
